SEQUENCE LISTING (15 sequences)

SEQ ID NO: 1 - protein, 1304 aa, Synthetic construct
VEDATRSDSTTQMSSTPEVVYSSAVDSKQNRTSDFDANWKFMLSDSVQAQDPAFDDSAWQQVDLPHDYSITQKYSQSNEAESAYLPGGTGWYRKSFTIDRDLAGKRIAINFDGVYMNATVWFNGVKLGTHPYGYSPFSFDLTGNAKFGGENTIVVKVENRLPSSRWYSGSGIYRDVTLTVTDGVHVGNNGVAIKTPSLATQNGGNVTMNLTTKVANDTKAAANITLKQTVFPKGGKTDAAIGTVTTASKSIAAGASADVTSTITAASPKLWSIKNPNLYTVRTEVLNGGKVLDTYDTEYGFRWTGFDATSGFSLNGEKVKLKGVSMHHDQGSLGAVANRRAIERQVEILQKMGVNSIRTTHNPAAKALIDVCNEKGVLVVEEVFDMWNRSKNGNTEDYGKWFGQAIAGDNAVLGGDKDETWAKFDLTSTINRDRNAPSVIMWSLGNEMMEGISGSVSGFPATSAKLVAWTKAADSTRPMTYGDNKIKANWNESNTMGDNLTANGGVVGTNYSDGANYDKIRTTHPSWAIYGSETASAINSRGIYNRTTGGAQSSDKQLTSYDNSAVGWGAVASSAWYDVVQRDFVAGTYVWTGFDYLGEPTPWNGTGSGAVGSWPSPKNSYFGIVDTAGFPKDTYYFYQSQWNDDVHTLHILPAWNENVVAKGSGNNVPVVVYTDAAKVKLYFTPKGSTEKRLIGEKSFTKKTTAAGYTYQVYEGADKDSTAHKNMYLTWNVPWAEGTISAEAYDENNRLIPEGSTEGNASVTTTGKAAKLKADADRKTITADGKDLSYIEVDVTDANGHIVPDAANRVTFDVKGAGKLVGVDNGSSPDHDSYQADNRKAFSGKVLAIVQSTKEAGEITVTAKADGLQSSTVKIATTAVPGTSTEKTVRSFYYSRNYYVKTGNKPILPSDVEVRYSDGTSDRQNVTWDAVSDDQIAKAGSFSVAGTVAGQKISVRVTMIDEIGALLNYSASTPVGTPAVLPGSRPAVLPDGTVTSANFAVDWTKPADTVYNTAGTVKVPGTATVFGKEFKVTATIRVQRSQVTIGSSVSGNALRLTQNIPADKQSDTLDAIKDGSTTVDANTGGGANPSAWTNWAYSKAGHNTAEITFEYATEQQLGQIVMYFFRDSNAVRFPDAGKTKIQISADGKNWTDLAATETIAAQESSERVKPYTYDFAPVGATFVKVTVTNADTTTPSGVVCAGLTEIELKTATSKFVTNTSAALSSLTVNGTKVSDSVLAAGSYNTPAIIADVKAEGEGNASVTVLPAHDNVIRVITESEDHVTRKTFTINLGTEQEFPADSDERD

SEQ ID NO: 2 - protein, 1931 aa, Synthetic construct
MKKPLGKIVASTALLISVAFSSSIASAAVEDATRSDSTTQMSSTPEVAYSSAVDSKQNRTSDFDANWKFMLSDSVQAQDPAFDDSAWQQVDLPHDYSITQKYSQSNEAESAYLPGGTGWYRKSFTIDRDLAGKRIAINFDGVYMNATVWFNGVKLGTHPYGYSPFSFDLTGNAKFGGENTIVVKVENRLPSSRWYSGSGIYRDVTLTVTDGVHVGNNGVAIKTPSLATQNGGDVTMNLTTKVANDTEAAANITLKQTVFPKGGKTDAAIGTVTTASKSIAAGASADVTSTITAASPKLWSIKNPNLYTVRTEVLNGGKVLDTYDTEYGFRWTGFDATSGFSLNGEKVKLKGVSMHHDQGSLGAVANRRAIERQVEILQKMGVNSIRTTHNPAAKALIDVCNEKGVLVVEEVFDMWNRSKNGNTEDYGKWFGQAIAGDNAVLGGDKDETWAKFDLTSTINRDRNAPSVIMWSLGNEMMEGISGSVSGFPATSAKLVAWTKAADSTRPMTYGDNKIKANWNESNTMGDNLTANGGVVGTNYSDGANYDKIRTTHPSWAIYGSETASAINSRGIYNRTTGGAQSSDKQLTSYDNSAVGWGAVASSAWYDVVQRDFVAGTYVWTGFDYLGEPTPWNGTGSGAVGSWPSPKNSYFGIVDTAGFPKDTYYFYQSQWNDDVHTLHILPAWNENVVAKGSGNNVPVVVYTDAAKVKLYFTPKGSTEKRLIGEKSFTKKTTAAGYTYQVYEGSDKDSTAHKNMYLTWNVPWAEGTISAEAYDENNRLIPEGSTEGNASVTTTGKAAKLKADADRKTITADGKDLSYIEVDVTDANGHIVPDAANRVTFDVKGAGKLVGVDNGSSPDHDSYQADNRKAFSGKVLAIVQSTKEAGEITVTAKADGLQSSTVKIATTAVPGTSTEKTVRSFYYSRNYYVKTGNKPILPSDVEVRYSDGTSDRQNVTWDAVSDDQIAKAGSFSVAGTVAGQKISVRVTMIDEIGALLNYSASTPVGTPAVLPGSRPAVLPDGTVTSANFAVHWTKPADTVYNTAGTVKVPGTATVFGKEFKVTATIRVQRSQVTIGSSVSGNALRLTQNIPADKQSDTLDAIKDGSTTVDANTGGGANPSAWTNWAYSKAGHNTAEITFEYATEQQLGQIVMYFFRDSNAVRFPDAGKTKIQISADGKNWTDLAATETIAAQESSDRVKPYTYDFAPVGATFVKVTVTNADTTTPSGVVCAGLTEIELKTATSKFVTNTSAALSSLTVNGTKVSDSVLAAGSYNTPAIIADVKAEGEGNASVTVLPAHDNVIRVITESEDHVTRKTFTINLGTEQEFPADSDERDYPAADMTVTVGSEQTSGTATEGPKKFAVDGNTSTYWHSNWTPTTVNDLWIAFELQKPTKLDALRYLPRPAGSKNGSVTEYKVQVSDDGTNWTDAGSGTWTTDYGWKLAEFNQPVTTKHVRLKAVHTYADSGNDKFMSASEIRLRKAVDTTDISGATVTVPAKLTVDRVDADHPATFATKDVTVTLGDATLRYGVDYLLDYAGNTAVGKATVTVRGIDKYSGTVAKTFTIELKNAPAPEPTLTSVSVKTKPSKLTYVVGDAFDPAGLVLQLNYDDDSTGTVTWNTQTAGDFTFKPALDAKLKVTDKTVTVTYQGKSAVIDITVSQPAPTVSKTDLDKAIKAIEAKNPDSSKYTADSWKTFADAMAHAKAVIADDSATQQDVDNALKALTDAYAGLTEKTPEPAPVSKSELDKKIKAIEAEKLDGSKYTAESWKAFETALAHAKAVIASDSATQQNVDAALGALTSARDGLTEKGEVKPDPKPEPGTVDKAALDKAVKKVEAEKLDGSKYTADSWKAFETALAHAKAVIGNANSTQFDIDNALSMLNDARAALKEKPGRIIAIIDGSALSKTGASVAIIASVAAAMLAVGAGVMALRRKRS

SEQ ID NO: 3 - protein, 1341 aa, Synthetic construct
MKKPLGKIVASTALLISVAFSSSIASAIEDATRSDSTTQMSSTPEVAYSSAVDSKQNRTSDFDANWKFMLSDSVQAQDPAFDDSAWQQVDLPHDYSITQKYSQSNEAESAYLPGGTGWYRKSFTIDRDLAGKRIAINFDGVYMNATVWFNGVKLGTHPYGYSPFSFDLTGNAKFGGENTIVVKVENRLPSSRWYSGSGIYRDVTLTVTDGVHVGNNGVAIKTPSLATQNGGDVTMNLTTKVANDTEAAANITLKQTVFPKGGKTDAAIGTVTTASKSIAAGASADVTSTITAASPKLWSIKNPNLYTVRTEVLNGGKVLDTYDTEYGFRWTGFDATSGFSLNGEKVKLKGVSMHHDQGSLGAVANRRAIERQVEILQKMGVNSIRTTHNPAAKALIDVCNEKGVLVVEEVFDMWNRSKNGNTEDYGKWFGQAIAGDNAVLGGDKDETWAKFDLTSTINRDRNAPSVIMWSLGNEMMEGISGSVSGFSATSAKLVAWTKAADSTRPMTYGDNKIKANWNESNTMGDNLTANGGVVGTNYSDGANYDKIRTTHPSWAIYGSETASAINSRGIYNRTTGGAQSSDKQLTSYDNSAVGWGAVASSAWYDVVQRDFVAGTYVWTGFDYLGEPTPWNGTGSGAVGSWPSPKNSYFGIVDTAGFPKDTYYFYQSQWNDDVHTLHILPAWNENVVAKGSGNNVPVVVYTDAAKVKLYFTPKGSTEQRLIGEKSFTKKTTAAGYTYQVYEGSDKDSTAHKNMYLTWNVPWAEGTISAEAYDENNRLIPEGSTEGNASVTTTGKAAKLKADADRKTITADGKDLSYIEVDVTDANGHIVPDAANRVTFDVKGAGKLVGVDNGSSPDHDSYQADNRKAFSGKVLAIVQSTKEAGEITVTAKADGLQSSTVKIATTAVPGTSTEKTVRSFYYSRNYYVKTGNKPILPSDVEVRYSDGTSDRQNVTWDAVSDDQIAKAGSFSVAGTVAGQKISVRVTMIDEIGALLNYSASTPVGTPAVLPGSRPAVLPDGTVTSANFAVHWTKPADTVYNTAGTVKVPGTATVFGKEFKVTATIRVQRSQVTIGSSVSGNALRLTQNIPADKQSDTLDAIKDGSTTVDANTGGGANPSAWTNWAYSKAGHNTAEITFEYATEQQLGQIVMYFFRDSNAVRFPDAGKTKIQISADGKNWTDLAATETIAAQESSDRVKPYTYDFAPVGATFVRVTVTNADTTTPSGVVCAGLTEIELKTATSKFVANTSAALSSLTVNGTKVSDSVLAAGSYNTPAIIADVKAEGEGNASVTVLPAHDNVIRVITESEDHVTRKTFTINLGTEQEFPADSDERDQHQHQHQHQQ

SEQ ID NO: 4 - protein, 1752 aa, Synthetic construct
MAVRRLGGRIVAFAATVALSIPLGLLTNSAWAVEDATRSDSTTQMSSTPEVVYSSAVDSKQNRTSDFDANWKFMLSDSVQAQDPAFDDSAWQQVDLPHDYSITQKYSQSNEAESAYLPGGTGWYRKSFTIDRDLAGKRIAINFDGVYMNATVWFNGVKLGTHPYGYSPFSFDLTGNAKFGGENTIVVKVENRLPSSRWYSGSGIYRDVTLTVTDGVHVGNNGVAIKTPSLATQNGGDVTMNLTTKVANDTEAAANITLKQTVFPKGGKTDAAIGTVTTASKSIAAGASADVTSTITAASPKLWSIKNPNLYTVRTEVLNGGKVLDTYDTEYGFRWTGFDATSGFSLNGEKVKLKGVSMHHDQGSLGAVANRRAIERQVEILQKMGVNSIRTTHNPAAKALIDVCNEKGVLVVEEVFDMWNRSKNGNTEDYGKWFGQAIAGDNAVLGGDKDETWAKFDLTSTINRDRNAPSVIMWSLGNEMMEGISGSVSGFPATSAKLVAWTKAADSTRPMTYGDNKIKANWNESNTMGDNLTANGGVVGTNYSDGANYDKIRTTHPSWAIYGSETASAINSRGIYNRTTGGAQSSDKQLTSYDNSAVGWGAVASSAWYDVVQRDFVAGTYVWTGFDYLGEPTPWNGTGSGAVGSWPSPKNSYFGIVDTAGFPKDTYYFYQSQWNDDVHTLHILPAWNENVVAKGSGNNVPVVVYTDAAKVKLYFTPKGSTEKRLIGEKSFTKKTTAAGYTYQVYEGSDKDSTAHKNMYLTWNVPWAEGTISAEAYDENNRLIPEGSTEGNASVTTTGKAAKLKADADRKTITADGKDLSYIEVDVTDANGHIVPDAANRVTFDVKGAGKLVGVDNGSSPDHDSYQADNRKAFSGKVLAIVQSTKEAGEITVTAKADGLQSSTVKIATTAVPGTSTEKTVRSFYYSRNYYVKTGNKPILPSDVEVRYSDGTSDRQNVTWDAVSDDQIAKAGSFSVAGTVAGQKISVRVTMIDEIGALLNYSASTPVGTPAVLPGSRPAVLPDGTVTSANFAVHWTKPADTVYNTAGTVKVPGTATVFGKEFKVTATIRVQRSQVTIGSSVSGNALRLTQNIPADKQSDTLDAIKDGSTTVDANTGGGANPSAWTNWAYSKAGHNTAEITFEYATEQQLGQIVMYFFRDSNAVRFPDAGKTKIQISADGKNWTDLAATETIAAQESSDRVKPYTYDFAPVGATFVKVTVTNADTTTPSGVVCAGLTEIELKTATSKFVTNTSAALSSLTVNGTKVSDSVLAAGSYNTPAIIADVKAEGEGNASVTVLPAHDNVIRVITESEDHVTRKTFTINLGTEQEFPADSDERDYPAADMTVTVGSEQTSGTATEGPKKFAVDGNTSTYWHSNWTPTTVNDLWIAFELQKPTKLDALRYLPRPAGSKNGSVTEYKVQVSDDGTNWTDAGSGTWTTDYGWKLAEFNQPVTTKHVRLKAVHTYADSGNDKFMSASEIRLRKAVDTTDISGATVTVPAKLTVDRVDADHPATFATKDVTVTLGDATLRYGVDYLLDYAGNTAVGKATVTVRGIDKYSGTVAKTFTIELKNAPAPEPTLTSVSVKTKPSKLTYVVGDAFDPAGLVLQHDRQADRPPQPLVGEQADERGLTCGTRCDRVEQLRKHENREAHRTGLDHLEFVGAADGAVGEQATFKVHVHADQGDGRHDDADERDIDPHVPVDHAVGELARAACHHVIGLRVDTHRLKASGFQIPADDMAEIDRITGFHRFERHVG

SEQ ID NO: 5 - protein, 1935 aa, Synthetic construct
MAVRRLGGRIVAFAATVALSIPLGLLTNSAWAVEDATRSDSTTQMSSTPEVVYSSAVDSKQNRTSDFDANWKFMLSDSVQAQDPAFDDSAWQQVDLPHDYSITQKYSQSNEAESAYLPGGTGWYRKSFTIDRDLAGKRIAINFDGVYMNATVWFNGVKLGTHPYGYSPFSFDLTGNAKFGGENTIVVKVENRLPSSRWYSGSGIYRDVTLTVTDGVHVGNNGVAIKTPSLATQNGGNVTMNLTTKVANDTKAAANITLKQTVFPKGGKTDAAIGTVTTASKSIAAGASADVTSTITAASPKLWSIKNPNLYTVRTEVLNGGKVLDTYDTEYGFRWTGFDATSGFSLNGEKVKLKGVSMHHDQGSLGAVANRRAIERQVEILQKMGVNSIRTTHNPAAKALIDVCNEKGVLVVEEVFDMWNRSKNGNTEDYGKWFGQAIAGDNAVLGGDKDETWAKFDLTSTINRDRNAPSVIMWSLGNEMMEGISGSVSGFPATSAKLVAWTKAADSTRPMTYGDNKIKANWNESNTMGDNLTANGGVVGTNYSDGANYDKIRTTHPSWAIYGSETASAINSRGIYNRTTGGAQSSDKQLTSYDNSAVGWGAVASSAWYDVVQRDFVAGTYVWTGFDYLGEPTPWNGTGSGAVGSWPSPKNSYFGIVDTAGFPKDTYYFYQSQWNDDVHTLHILPAWNENVVAKGSGNNVPVVVYTDAAKVKLYFTPKGSTEKRLIGEKSFTKKTTAAGYTYQVYEGADKDSTAHKNMYLTWNVPWAEGTISAEAYDENNRLIPEGSTEGNASVTTTGKAAKLKADADRKTITADGKDLSYIEVDVTDANGHIVPDAANRVTFDVKGAGKLVGVDNGSSPDHDSYQADNRKAFSGKVLAIVQSTKEAGEITVTAKADGLQSSTVKIATTAVPGTSTEKTVRSFYYSRNYYVKTGNKPILPSDVEVRYSDGTSDRQNVTWDAVSDDQIAKAGSFSVAGTVAGQKISVRVTMIDEIGALLNYSASTPVGTPAVLPGSRPAVLPDGTVTSANFAVDWTKPADTVYNTAGTVKVPGTATVFGKEFKVTATIRVQRSQVTIGSSVSGNALRLTQNIPADKQSDTLDAIKDGSTTVDANTGGGANPSAWTNWAYSKAGHNTAEITFEYATEQQLGQIVMYFFRDSNAVRFPDAGKTKIQISADGKNWTDLAATETIAAQESSDRVKPYTYDFAPVGATFVKVTVTNADTTTPSGVVCAGLTEIELKTATSKFVTNTSAALSSLTVNGTKVSDSVLAAGSYNTPAIIADVKAEGEGNASVTVLPAHDNVIRVITESEDHVTRKTFTINLGTEQEFPADSDERDYPAADMTVTAGSEQTSGTATEGPKKFAVDGNTSTYWHSNWTPTTVNDLWIAFELQKPTKLDALRYLPRPAGSKNGSVTEYKVQVSDDGTNWTDAGSGTWTTDYGWKLAEFNQPVTTKHVRLKAVHTYADSGNDKFMSASEIRLRKAVDTTDISGATVTVPAKLTVDRVDADHPATFATKDVTVTLGDATLRYGVDYLLDYAGNTAVGKATVTVRGIDKYSGTVAKTFTIELKNAPAPEPTLTSVSVKTKPSKLTYVVGDAFDPAGLVLQLNYDDDSTGTVTWNTQTAGDFTFKPALDAKLKVTDKTVTVTYQGKSAVIDITVSQPAPTVSKTDLDKAIKAIEAKNPDSSKYTADSWKTFADAMAHAKAVIADDSATQQDVDKALKALTDAYAGLTEKTPEPAPVSKSELDKKIKAIEAEKLDGSKYTAESWKAFETALAHAKAVIASDSATQQDVDAALGALTSARDGLTEKGEVKPDPKPEPGTVDKAALDKAVKKVEAEKLDGSKYTADSWKAFETALAHAKAVIGNANSTQFDIDNALSMLNDARAALKEKPGRIIAIIDGGALSKTGASVAIIASVAAAMKAVGAGVMALRPPKW

SEQ ID NO: 6 - protein, 887 aa, Bifidobacterium bifidum
VEDATRSDSTTQMSSTPEVVYSSAVDSKQNRTSDFDANWKFMLSDSVQAQDPAFDDSAWQQVDLPHDYSITQKYSQSNEAESAYLPGGTGWYRKSFTIDRDLAGKRIAINFDGVYMNATVWFNGVKLGTHPYGYSPFSFDLTGNAKFGGENTIVVKVENRLPSSRWYSGSGIYRDVTLTVTDGVHVGNNGVAIKTPSLATQNGGDVTMNLTTKVANDTEAAANITLKQTVFPKGGKTDAAIGTVTTASKSIAAGASADVTSTITAASPKLWSIKNPNLYTVRTEVLNGGKVLDTYDTEYGFRWTGFDATSGFSLNGEKVKLKGVSMHHDQGSLGAVANRRAIERQVEILQKMGVNSIRTTHNPAAKALIDVCNEKGVLVVEEVFDMWNRSKNGNTEDYGKWFGQAIAGDNAVLGGDKDETWAKFDLTSTINRDRNAPSVIMWSLGNEMMEGISGSVSGFPATSAKLVAWTKAADSTRPMTYGDNKIKANWNESNTMGDNLTANGGVVGTNYSDGANYDKIRTTHPSWAIYGSETASAINSRGIYNRTTGGAQSSDKQLTSYDNSAVGWGAVASSAWYDVVQRDFVAGTYVWTGFDYLGEPTPWNGTGSGAVGSWPSPKNSYFGIVDTAGFPKDTYYFYQSQWNDDVHTLHILPAWNENVVAKGSGNNVPVVVYTDAAKVKLYFTPKGSTEKRLIGEKSFTKKTTAAGYTYQVYEGSDKDSTAHKNMYLTWNVPWAEGTISAEAYDENNRLIPEGSTEGNASVTTTGKAAKLKADADRKTITADGKDLSYIEVDVTDANGHIVPDAANRVTFDVKGAGKLVGVDNGSSPDHDSYQADNRKAFSGKVLAIVQSTKEAGEITVTAKADGLQSSTVKIATTAVPGTSTEKT

SEQ ID NO: 7 - protein, 965 aa, Bifidobacterium bifidum
VEDATRSDSTTQMSSTPEVVYSSAVDSKQNRTSDFDANWKFMLSDSVQAQDPAFDDSAWQQVDLPHDYSITQKYSQSNEAESAYLPGGTGWYRKSFTIDRDLAGKRIAINFDGVYMNATVWFNGVKLGTHPYGYSPFSFDLTGNAKFGGENTIVVKVENRLPSSRWYSGSGIYRDVTLTVTDGVHVGNNGVAIKTPSLATQNGGDVTMNLTTKVANDTEAAANITLKQTVFPKGGKTDAAIGTVTTASKSIAAGASADVTSTITAASPKLWSIKNPNLYTVRTEVLNGGKVLDTYDTEYGFRWTGFDATSGFSLNGEKVKLKGVSMHHDQGSLGAVANRRAIERQVEILQKMGVNSIRTTHNPAAKALIDVCNEKGVLVVEEVFDMWNRSKNGNTEDYGKWFGQAIAGDNAVLGGDKDETWAKFDLTSTINRDRNAPSVIMWSLGNEMMEGISGSVSGFPATSAKLVAWTKAADSTRPMTYGDNKIKANWNESNTMGDNLTANGGVVGTNYSDGANYDKIRTTHPSWAIYGSETASAINSRGIYNRTTGGAQSSDKQLTSYDNSAVGWGAVASSAWYDVVQRDFVAGTYVWTGFDYLGEPTPWNGTGSGAVGSWPSPKNSYFGIVDTAGFPKDTYYFYQSQWNDDVHTLHILPAWNENVVAKGSGNNVPVVVYTDAAKVKLYFTPKGSTEKRLIGEKSFTKKTTAAGYTYQVYEGSDKDSTAHKNMYLTWNVPWAEGTISAEAYDENNRLIPEGSTEGNASVTTTGKAAKLKADADRKTITADGKDLSYIEVDVTDANGHIVPDAANRVTFDVKGAGKLVGVDNGSSPDHDSYQADNRKAFSGKVLAIVQSTKEAGEITVTAKADGLQSSTVKIATTAVPGTSTEKTVRSFYYSRNYYVKTGNKPILPSDVEVRYSDGTSDRQNVTWDAVSDDQIAKAGSFSVAGTVAGQKISVRVTMIDEIGAL

SEQ ID NO: 8 - protein, 1038 aa, Bifidobacterium bifidum
VEDATRSDSTTQMSSTPEVVYSSAVDSKQNRTSDFDANWKFMLSDSVQAQDPAFDDSAWQQVDLPHDYSITQKYSQSNEAESAYLPGGTGWYRKSFTIDRDLAGKRIAINFDGVYMNATVWFNGVKLGTHPYGYSPFSFDLTGNAKFGGENTIVVKVENRLPSSRWYSGSGIYRDVTLTVTDGVHVGNNGVAIKTPSLATQNGGDVTMNLTTKVANDTEAAANITLKQTVFPKGGKTDAAIGTVTTASKSIAAGASADVTSTITAASPKLWSIKNPNLYTVRTEVLNGGKVLDTYDTEYGFRWTGFDATSGFSLNGEKVKLKGVSMHHDQGSLGAVANRRAIERQVEILQKMGVNSIRTTHNPAAKALIDVCNEKGVLVVEEVFDMWNRSKNGNTEDYGKWFGQAIAGDNAVLGGDKDETWAKFDLTSTINRDRNAPSVIMWSLGNEMMEGISGSVSGFPATSAKLVAWTKAADSTRPMTYGDNKIKANWNESNTMGDNLTANGGVVGTNYSDGANYDKIRTTHPSWAIYGSETASAINSRGIYNRTTGGAQSSDKQLTSYDNSAVGWGAVASSAWYDVVQRDFVAGTYVWTGFDYLGEPTPWNGTGSGAVGSWPSPKNSYFGIVDTAGFPKDTYYFYQSQWNDDVHTLHILPAWNENVVAKGSGNNVPVVVYTDAAKVKLYFTPKGSTEKRLIGEKSFTKKTTAAGYTYQVYEGSDKDSTAHKNMYLTWNVPWAEGTISAEAYDENNRLIPEGSTEGNASVTTTGKAAKLKADADRKTITADGKDLSYIEVDVTDANGHIVPDAANRVTFDVKGAGKLVGVDNGSSPDHDSYQADNRKAFSGKVLAIVQSTKEAGEITVTAKADGLQSSTVKIATTAVPGTSTEKTVRSFYYSRNYYVKTGNKPILPSDVEVRYSDGTSDRQNVTWDAVSDDQIAKAGSFSVAGTVAGQKISVRVTMIDEIGALLNYSASTPVGTPAVLPGSRPAVLPDGTVTSANFAVHWTKPADTVYNTAGTVKVPGTATVFGKEFKVTATIRVQ

SEQ ID NO: 9 - protein, 1142 aa, Bifidobacterium bifidum
VEDATRSDSTTQMSSTPEVVYSSAVDSKQNRTSDFDANWKFMLSDSVQAQDPAFDDSAWQQVDLPHDYSITQKYSQSNEAESAYLPGGTGWYRKSFTIDRDLAGKRIAINFDGVYMNATVWFNGVKLGTHPYGYSPFSFDLTGNAKFGGENTIVVKVENRLPSSRWYSGSGIYRDVTLTVTDGVHVGNNGVAIKTPSLATQNGGDVTMNLTTKVANDTEAAANITLKQTVFPKGGKTDAAIGTVTTASKSIAAGASADVTSTITAASPKLWSIKNPNLYTVRTEVLNGGKVLDTYDTEYGFRWTGFDATSGFSLNGEKVKLKGVSMHHDQGSLGAVANRRAIERQVEILQKMGVNSIRTTHNPAAKALIDVCNEKGVLVVEEVFDMWNRSKNGNTEDYGKWFGQAIAGDNAVLGGDKDETWAKFDLTSTINRDRNAPSVIMWSLGNEMMEGISGSVSGFPATSAKLVAWTKAADSTRPMTYGDNKIKANWNESNTMGDNLTANGGVVGTNYSDGANYDKIRTTHPSWAIYGSETASAINSRGIYNRTTGGAQSSDKQLTSYDNSAVGWGAVASSAWYDVVQRDFVAGTYVWTGFDYLGEPTPWNGTGSGAVGSWPSPKNSYFGIVDTAGFPKDTYYFYQSQWNDDVHTLHILPAWNENVVAKGSGNNVPVVVYTDAAKVKLYFTPKGSTEKRLIGEKSFTKKTTAAGYTYQVYEGSDKDSTAHKNMYLTWNVPWAEGTISAEAYDENNRLIPEGSTEGNASVTTTGKAAKLKADADRKTITADGKDLSYIEVDVTDANGHIVPDAANRVTFDVKGAGKLVGVDNGSSPDHDSYQADNRKAFSGKVLAIVQSTKEAGEITVTAKADGLQSSTVKIATTAVPGTSTEKTVRSFYYSRNYYVKTGNKPILPSDVEVRYSDGTSDRQNVTWDAVSDDQIAKAGSFSVAGTVAGQKISVRVTMIDEIGALLNYSASTPVGTPAVLPGSRPAVLPDGTVTSANFAVHWTKPADTVYNTAGTVKVPGTATVFGKEFKVTATIRVQRSQVTIGSSVSGNALRLTQNIPADKQSDTLDAIKDGSTTVDANTGGGANPSAWTNWAYSKAGHNTAEITFEYATEQQLGQIVMYFFRDSNAVRFPDAGKTKIQI

SEQ ID NO: 10 - protein, 1211 aa, Bifidobacterium bifidum
VEDATRSDSTTQMSSTPEVVYSSAVDSKQNRTSDFDANWKFMLSDSVQAQDPAFDDSAWQQVDLPHDYSITQKYSQSNEAESAYLPGGTGWYRKSFTIDRDLAGKRIAINFDGVYMNATVWFNGVKLGTHPYGYSPFSFDLTGNAKFGGENTIVVKVENRLPSSRWYSGSGIYRDVTLTVTDGVHVGNNGVAIKTPSLATQNGGDVTMNLTTKVANDTEAAANITLKQTVFPKGGKTDAAIGTVTTASKSIAAGASADVTSTITAASPKLWSIKNPNLYTVRTEVLNGGKVLDTYDTEYGFRWTGFDATSGFSLNGEKVKLKGVSMHHDQGSLGAVANRRAIERQVEILQKMGVNSIRTTHNPAAKALIDVCNEKGVLVVEEVFDMWNRSKNGNTEDYGKWFGQAIAGDNAVLGGDKDETWAKFDLTSTINRDRNAPSVIMWSLGNEMMEGISGSVSGFPATSAKLVAWTKAADSTRPMTYGDNKIKANWNESNTMGDNLTANGGVVGTNYSDGANYDKIRTTHPSWAIYGSETASAINSRGIYNRTTGGAQSSDKQLTSYDNSAVGWGAVASSAWYDVVQRDFVAGTYVWTGFDYLGEPTPWNGTGSGAVGSWPSPKNSYFGIVDTAGFPKDTYYFYQSQWNDDVHTLHILPAWNENVVAKGSGNNVPVVVYTDAAKVKLYFTPKGSTEKRLIGEKSFTKKTTAAGYTYQVYEGSDKDSTAHKNMYLTWNVPWAEGTISAEAYDENNRLIPEGSTEGNASVTTTGKAAKLKADADRKTITADGKDLSYIEVDVTDANGHIVPDAANRVTFDVKGAGKLVGVDNGSSPDHDSYQADNRKAFSGKVLAIVQSTKEAGEITVTAKADGLQSSTVKIATTAVPGTSTEKTVRSFYYSRNYYVKTGNKPILPSDVEVRYSDGTSDRQNVTWDAVSDDQIAKAGSFSVAGTVAGQKISVRVTMIDEIGALLNYSASTPVGTPAVLPGSRPAVLPDGTVTSANFAVHWTKPADTVYNTAGTVKVPGTATVFGKEFKVTATIRVQRSQVTIGSSVSGNALRLTQNIPADKQSDTLDAIKDGSTTVDANTGGGANPSAWTNWAYSKAGHNTAEITFEYATEQQLGQIVMYFFRDSNAVRFPDAGKTKIQISADGKNWTDLAATETIAAQESSDRVKPYTYDFAPVGATFVKVTVTNADTTTPSGVVCAGLTEIELKTAT

SEQ ID NO: 11 - protein, 1296 aa, Bifidobacterium bifidum
VEDATRSDSTTQMSSTPEVVYSSAVDSKQNRTSDFDANWKFMLSDSVQAQDPAFDDSAWQQVDLPHDYSITQKYSQSNEAESAYLPGGTGWYRKSFTIDRDLAGKRIAINFDGVYMNATVWFNGVKLGTHPYGYSPFSFDLTGNAKFGGENTIVVKVENRLPSSRWYSGSGIYRDVTLTVTDGVHVGNNGVAIKTPSLATQNGGDVTMNLTTKVANDTEAAANITLKQTVFPKGGKTDAAIGTVTTASKSIAAGASADVTSTITAASPKLWSIKNPNLYTVRTEVLNGGKVLDTYDTEYGFRWTGFDATSGFSLNGEKVKLKGVSMHHDQGSLGAVANRRAIERQVEILQKMGVNSIRTTHNPAAKALIDVCNEKGVLVVEEVFDMWNRSKNGNTEDYGKWFGQAIAGDNAVLGGDKDETWAKFDLTSTINRDRNAPSVIMWSLGNEMMEGISGSVSGFPATSAKLVAWTKAADSTRPMTYGDNKIKANWNESNTMGDNLTANGGVVGTNYSDGANYDKIRTTHPSWAIYGSETASAINSRGIYNRTTGGAQSSDKQLTSYDNSAVGWGAVASSAWYDVVQRDFVAGTYVWTGFDYLGEPTPWNGTGSGAVGSWPSPKNSYFGIVDTAGFPKDTYYFYQSQWNDDVHTLHILPAWNENVVAKGSGNNVPVVVYTDAAKVKLYFTPKGSTEKRLIGEKSFTKKTTAAGYTYQVYEGSDKDSTAHKNMYLTWNVPWAEGTISAEAYDENNRLIPEGSTEGNASVTTTGKAAKLKADADRKTITADGKDLSYIEVDVTDANGHIVPDAANRVTFDVKGAGKLVGVDNGSSPDHDSYQADNRKAFSGKVLAIVQSTKEAGEITVTAKADGLQSSTVKIATTAVPGTSTEKTVRSFYYSRNYYVKTGNKPILPSDVEVRYSDGTSDRQNVTWDAVSDDQIAKAGSFSVAGTVAGQKISVRVTMIDEIGALLNYSASTPVGTPAVLPGSRPAVLPDGTVTSANFAVHWTKPADTVYNTAGTVKVPGTATVFGKEFKVTATIRVQRSQVTIGSSVSGNALRLTQNIPADKQSDTLDAIKDGSTTVDANTGGGANPSAWTNWAYSKAGHNTAEITFEYATEQQLGQIVMYFFRDSNAVRFPDAGKTKIQISADGKNWTDLAATETIAAQESSDRVKPYTYDFAPVGATFVKVTVTNADTTTPSGVVCAGLTEIELKTATSKFVTNTSAALSSLTVNGTKVSDSVLAAGSYNTPAIIADVKAEGEGNASVTVLPAHDNVIRVITESEDHVTRKTFTINLGTEQEF

SEQ ID NO: 12 - protein, 1720 aa, Bifidobacterium bifidum
VEDATRSDSTTQMSSTPEVVYSSAVDSKQNRTSDFDANWKFMLSDSVQAQDPAFDDSAWQQVDLPHDYSITQKYSQSNEAESAYLPGGTGWYRKSFTIDRDLAGKRIAINFDGVYMNATVWFNGVKLGTHPYGYSPFSFDLTGNAKFGGENTIVVKVENRLPSSRWYSGSGIYRDVTLTVTDGVHVGNNGVAIKTPSLATQNGGDVTMNLTTKVANDTEAAANITLKQTVFPKGGKTDAAIGTVTTASKSIAAGASADVTSTITAASPKLWSIKNPNLYTVRTEVLNGGKVLDTYDTEYGFRWTGFDATSGFSLNGEKVKLKGVSMHHDQGSLGAVANRRAIERQVEILQKMGVNSIRTTHNPAAKALIDVCNEKGVLVVEEVFDMWNRSKNGNTEDYGKWFGQAIAGDNAVLGGDKDETWAKFDLTSTINRDRNAPSVIMWSLGNEMMEGISGSVSGFPATSAKLVAWTKAADSTRPMTYGDNKIKANWNESNTMGDNLTANGGVVGTNYSDGANYDKIRTTHPSWAIYGSETASAINSRGIYNRTTGGAQSSDKQLTSYDNSAVGWGAVASSAWYDVVQRDFVAGTYVWTGFDYLGEPTPWNGTGSGAVGSWPSPKNSYFGIVDTAGFPKDTYYFYQSQWNDDVHTLHILPAWNENVVAKGSGNNVPVVVYTDAAKVKLYFTPKGSTEKRLIGEKSFTKKTTAAGYTYQVYEGSDKDSTAHKNMYLTWNVPWAEGTISAEAYDENNRLIPEGSTEGNASVTTTGKAAKLKADADRKTITADGKDLSYIEVDVTDANGHIVPDAANRVTFDVKGAGKLVGVDNGSSPDHDSYQADNRKAFSGKVLAIVQSTKEAGEITVTAKADGLQSSTVKIATTAVPGTSTEKTVRSFYYSRNYYVKTGNKPILPSDVEVRYSDGTSDRQNVTWDAVSDDQIAKAGSFSVAGTVAGQKISVRVTMIDEIGALLNYSASTPVGTPAVLPGSRPAVLPDGTVTSANFAVHWTKPADTVYNTAGTVKVPGTATVFGKEFKVTATIRVQRSQVTIGSSVSGNALRLTQNIPADKQSDTLDAIKDGSTTVDANTGGGANPSAWTNWAYSKAGHNTAEITFEYATEQQLGQIVMYFFRDSNAVRFPDAGKTKIQISADGKNWTDLAATETIAAQESSDRVKPYTYDFAPVGATFVKVTVTNADTTTPSGVVCAGLTEIELKTATSKFVTNTSAALSSLTVNGTKVSDSVLAAGSYNTPAIIADVKAEGEGNASVTVLPAHDNVIRVITESEDHVTRKTFTINLGTEQEFPADSDERDYPAADMTVTVGSEQTSGTATEGPKKFAVDGNTSTYWHSNWTPTTVNDLWIAFELQKPTKLDALRYLPRPAGSKNGSVTEYKVQVSDDGTNWTDAGSGTWTTDYGWKLAEFNQPVTTKHVRLKAVHTYADSGNDKFMSASEIRLRKAVDTTDISGATVTVPAKLTVDRVDADHPATFATKDVTVTLGDATLRYGVDYLLDYAGNTAVGKATVTVRGIDKYSGTVAKTFTIELKNAPAPEPTLTSVSVKTKPSKLTYVVGDAFDPAGLVLQHDRQADRPPQPLVGEQADERGLTCGTRCDRVEQLRKHENREAHRTGLDHLEFVGAADGAVGEQATFKVHVHADQGDGRHDDADERDIDPHVPVDHAVGELARAACHHVIGLRVDTHRLKASGFQIPADDMAEIDRITGFHRFERHVG

SEQ ID NO: 13 - protein, 1396 aa, Bacillus circulans
MRRINFNDNWRFQREISTSLREAQKPSFNDHSWRQLSLPHDWSIELDFNKDSLATHEGGYLDGGVGWYRKTFTVPSAMEGKRISLDFDGVYMNSTTYLNGEELGTYPFGYNAFSYDITDKLFMDGRENVLAVKVDNTQPSSRWYSGSGIYRNVYLTVTNPVHVARYGTFVTTPDLESAYAARKAEVNIKTKINNDSDAAVQVKVKSTIYDTDGKEVASVVSQEKTAAAGTTAHFEDNTVIENPELWSLDNPYRYKLVTDVLIGGETVDTYETRFGARFFKFDANEGFSLNGKPMKLYGVSMHHDLGALGAATNARAVERQLQIMKDMGVNAIRGTHNPVSPEFLEAVNNLGLLLIEEAFDCWSQSKKTYDYGRFFTRWAEHDVKEMVDRGKNEPSIIMWSIGNEIYDTTSPSGVETARNLVRWIKEIDTTRPTTIGEDKTRGDKVNVTPIDPNILEIFHTVDVVGLNYSENNYVGYHEQHPNWKLYGSETSSATRSRGVYTHPYEYNLGTKYDDLQQSSYDNDYVPWGRTAEDAWKSDRDLKHFAGQFIWTGFDYIGEPTPYYDSYPAKSSYFGAVDTAGFPKDIFYYYQSQWKKEPMVHLLPHWNWTEGEPVRVLAYTNAHQVELFLNGKSLGVRGYENKKTSWGAPYKETKDGKTYLEWAVPFKAGTLEAVAMDENGKEIARDQVTTAGAPAAVKLTADRKVIKADGTDLSFITAEIVDSKGNVVPNADHLIQFHLSGHGELAGVDNGDAASVERYKDNKRKAFSGKALAIVQSNKLDGNITLHASAEGLSSGNVTIFTTASADQNSITIAGIDEVNVLVDFNVVPELPSQIKVYYSDSTVEMKPVTWDAVDPNLLNTAGKIIVEGTVEGTDKKAKALLIVKGNGQENSEYRIDLFSPDPKLISTELTVEKTNIMEDDFIDIKVIGQLENKEVVDLSNFMPIYEFDCDIIKIEGNKLYALEEGLVKVTAAVTYKGRTVTSPEMMLKITKNPVPKTITHIDSITVVAGKGEAPVLPATAVAHFDRGMPRDVKVKWEIVNPALYQNLGEFTVSGDVEGTEIKAQAKVMVRSALAIETISMAVLPNQKPELPQKVTVYYSDGTEEQADVDWDAMPSAELKSEGVVKVKGSVKGVDLKATAQIRVTSEVGGVQNISRAKNGYEYPKAEASFTNTGPGSNDRIEAINDDVISYDAEPHNRWTNWQPTPRPGDWVSITFGDSKPRKYDIDSMEIHWYEDLGTSSPAYFRIQYKSGDEWKDVSGLKTNPSNTVLRQANVYTFDKVRTSAIRVDMTAKTGKSLAITEIKVFSKWAKAHTHPMVTDIKLGDLSILDDFSKKGDNNELTFQVKDPRDIPEIKVKAEDNTSITIIPTFTAPSTAKIIAKSEDGMKVEIYNIRFTE

SEQ ID NO: 14 - protein, 1737 aa, Bacillus sp.
MKKAISCVFLISALILSSFQVPVQGQAMSKTTSAAGNSVSYDGERRVNFNENWRFQRETNGSIAGAQNPGFDDSSWRKLNLPHDWSIELDFNKNSLATHEGGYLDGGIGWYRKTFTIPESMKGKRISLDFDGVYMNSTTYLNGEVLGTYPFGYNAFSYDISDKLYKDGRANVLVVKVNNTQPSSRWYSGSGIYRNVYLTVTDPIHVARYGTFVTTPNLEKSIKEDRADVNIKTKISNDAAEAKQVKIKSTIYDGAGNTVQTVETEEKTAAAGTVTPFEQNTVIKQPKLWSIDKPYRYNLVTEVIVGGQTVDTYETKFGVRYFKFDENEGFSLNGEYMKLHGVSMHHDLGALGAATNARGVERQMQIMKDMGVNAIRVTHNPASPELLEAANKLGLFIIEEAFDSWAQSKKPYDYGRFFNAWAEHDIKEMVDRGKNEPAIIMWSIGNEIYDTTNAAGVETARNLVGWVKEIDTTRPTTIGEDKTRGDKVNVTPINSYIKEIFNIVDVVGLNYSENNYDGYHKQNPSWKLYGSETSSATRSRGVYTHPYQYNQSTKYADLQQSSYDNDYVGWGRTAEDAWKYDRDLKHIAGQFIWTGFDYIGEPTPYYNSYPAKSSYFGAVDTAGFPKDIFYYYQSQWKKEPMVHLLPHWNWKEGEKVRVLAYTNASKVELVLNGESLGEKNYDNKQTSWGAPYKETKDGKTYLEWAVPFKPGKLEAVAKDENGKVIARDQVVTAGEPASVRLTADRKVVKADGTDLSFITADIVDSKGIVVPDADHLITFNVTGQGELAGVDNGNASSVERYKDNKRKAFSGKALAIVQSSKLSGKITVHASVAGLSSDSTSVFTVTPADHDKKIVAGIDDVNLTVDVNEAPKLPSEIKVYYSDESAAAKNVTWDEVDPKQYSTVGEFTVEGSVEGTSLKAKAFVIVKGIVAVKPYSTATKVGVQPVLPEKATLLYSDGTTKGATVTWDEIPEDKLAKEGRFTVEGSVEGTDLKANVYVRVTNEVKSVNIMLQEQGSAYPKLEATFTNPADNLQHLNDGIKSYTNNPVNRWTNWTRTPRDAGDSITVNFGKKHVINNLDLFVFTDSGTVVPEKAEVQYWDGTAWKDVENLTQPSPYVVEKNELTFDAVATEKLKFHLTPSVKGKFLALTEAEVYADQIVMGETAKLQSITVNGKALEGFDHAKKNYELVLPYGSELPKIEAAAADNATVTILPAFSYPGTAKLFVTSEDGKVTTEYSIGVSTEEPKLVSAELSADKTNVMEDDIIDLKVIGLFESKEKIDVTDSQPTYEFDQQIIKIEGNKLYALETGNVKVKVTVTYKGVSVTTPALEFTIAKNPAPKYITSLEPVTVVVKKGEAPELPATVVAHYNRGIPRDVKVKWERINPSKYQQLGEFTVSGMVEGTDIKAQAKVIVKGAVAVEDIRMAVLLKQMPQLPGKVTVYYSDGAEEQRAVKWEEIPQEELENVGEFKVKGDVNGVKLKATATIRVTDEVGGEQNISRAKNGYEYPKAEASFTNNGPGSSDRIEAINDDVISYEANPHNRWTNWQPVPRAGDWVSITFGDYEPTEYDVDSMEIHWFADHGTSYPERFQIEYKSGDSWKEVTSLKSDPASPALGKANVYSFDRVKTSAIRVKMTAQAGKSLAITELKVFSKWPKAGTEPEVTDIKVGGKSILEDFEQKGDHYEVTIDAGDANVMPKINVKAKDQTSITIVPAVTSPSTAKVIAKSEDGKKVKVYSIHYK

SEQ ID NO: 15 - protein, 1025 aa, Kluyveromyces lactis
MSCLIPENLRNPKKVHENRLPTRAYYYDQDIFESLNGPWAFALFDAPLDAPDAKNLDWETAKKWSTISVPSHWELQEDWKYGKPIYTNVQYPIPIDIPNPPTVNPTGVYARTFELDSKSIESFEHRLRFEGVDNCYELYVNGQYVGFNKGSRNGAEFDIQKYVSEGENLVVVKVFKWSDSTYIEDQDQWWLSGIYRDVSLLKLPKKAHIEDVRVTTTFVDSQYQDAELSVKVDVQGSSYDHINFTLYEPEDGSKVYDASSLLNEENGNTTFSTKEFISFSTKKNEETAFKINVKAPEHWTAENPTLYKYQLDLIGSDGSVIQSIKHHVGFRQVELKDGNITVNGKDILFRGVNRHDHHPRFGRAVPLDFVVRDLILMKKFNINAVRNSHYPNHPKVYDLFDKLGFWVIDEADLETHGVQEPFNRHTNLEAEYPDTKNKLYDVNAHYLSDNPEYEVAYLDRASQLVLRDVNHPSIIIWSLGNEACYGRNHKAMYKLIKQLDPTRLVHYEGDLNALSADIFSFMYPTFEIMERWRKNHTDENGKFEKPLILCEYGHAMGNGPGSLKEYQELFYKEKFYQGGFIWEWANHGIEFEDVSTADGKLHKAYAYGGDFKEEVHDGVFIMDGLCNSEHNPTPGLVEYKKVIEPVHIKIAHGSVTITNKHDFITTDHLLFIDKDTGKTIDVPSLKPEESVTIPSDTTYVVAVLKDDAGVLKAGHEIAWGQAELPLKVPDFVTETAEKAAKINDGKRYVSVESSGLHFILDKLLGKIESLKVKGKEISSKFEGSSITFWRPPTNNDEPRDFKNWKKYNIDLMKQNIHGVSVEKGSNGSLAVVTVNSRISPVVFYYGFETVQKYTIFANKINLNTSMKLTGEYQPPDFPRVGYEFWLGDSYESFEWLGRGPGESYPDKKESQRFGLYDSKDVEEFVYDYPQENGNHTDTHFLNIKFEGAGKLSIFQKEKPFNFKISDEYGVDEAAHACDVKRYGRHYLRLDHAIHGVGSEACGPAVLDQYRLKAQDFNFEFDLAFE

(19) United States
(12) Patent Application Publication
(10) Pub. No.: US 2026/0109961 A1
(43) Pub. Date: Apr. 23, 2026

(54) POLYPEPTIDE VARIANTS

(71) Applicant: NOVOZYMES A/S, Bagsvaerd (DK)

(72) Inventors: Roland Alexander PACHE, Valby (DK); Anders Gunnar SANDSTROM, Sandby (SE); Jan Kjoelhede VESTER, Hvidovre (DK); Henrik FRIIS-MADSEN, Ballerup (DK); Dorota NISSEN, Vedbaek (DK)

(73) Assignee: NOVOZYMES A/S, Bagsvaerd (DK)

(21) Appl. No.: 19/230,791

(22) Filed: Jun. 6, 2025

Related U.S. Application Data
(62) Division of application No. 17/759,537, now Pat. No. 12,359,182.

(30) Foreign Application Priority Data
Feb. 26, 2020 (EP) 20159590.7

(51) Int. Cl. C12N 9/38; A23C 9/12; C12N 15/52; C12N 15/63

(52) U.S. Cl. CPC C12N 9/2471; A23C 9/1206; C12N 15/52; C12N 15/63; C12Y 302/01023

(57) ABSTRACT

The present invention relates to novel lactase variants having improved properties relative to the parent lactase, such as decreased galactose inhibition or increased galactose inhibition. The variants of the invention are suitable for production of dairy products, such as low-lactose dairy products or lactose-free dairy products. The present invention also relates to isolated DNA sequences encoding the variants, expression vectors, host cells, and methods for producing and using the lactase variants of the invention.

### CROSS-REFERENCE TO RELATED APPLICATIONS

This application is a divisional of U.S. application Ser. No. 17/759,537 filed on Jul. 27, 2022 (now pending) which is a 35 U.S.C. 371 national application of international application no. PCT/EP2021/054398 filed Feb. 23, 2021, which claims priority or the benefit under 35 U.S.C. 119 of European application no. 20159590.7 filed Feb. 26, 2020. The content of each application is fully incorporated herein by reference.

### REFERENCE TO SEQUENCE LISTING

This application contains a Sequence Listing in computer readable form. The computer readable form is incorporated herein by reference. The contents of the electronic sequence listing created on Jun. 4, 2025, named SQ.xml and is 34,669 bytes in size, is hereby incorporated by reference in its entirety.

### FIELD OF THE INVENTION

The present invention relates to novel lactase variants having improved properties relative to the parent lactase, such as decreased galactose inhibition or increased galactose inhibition. The variants of the invention are suitable for production of dairy products, such as low-lactose dairy products or lactose-free dairy products. The present invention also relates to isolated DNA sequences encoding the variants, expression vectors, host cells, and methods for producing and using the lactase variants of the invention.

### BACKGROUND OF THE INVENTION

Lactase is used commercially to hydrolyze lactose in milk into its monosaccharides glucose and galactose. Low-lactose or lactose-free dairy products can thus be obtained which are suitable for people with lactose intolerance. Another advantage of low-lactose or lactose-free dairy products is that they have a sweeter taste since glucose and galactose are sweeter than lactose. Dairy products having a sweeter taste can thus be obtained without the addition of any sugar, artificial sweetener or the like. Lactase is also used in the manufacture of ice cream. Lactose crystallises at the low temperatures of ice cream, whereas glucose and galactose stay liquid and contribute to a smoother texture. Lactase is also used in the conversion of whey into syrup. Lactase is also used for production of condensed milk.

WO2009/071539 discloses a C-terminally truncated fragment of an extracellular lactase from Bifidobacterium bifidum, which hydrolyzes lactose very efficiently and enables the achievement of low levels of lactose in the dairy product. The enzyme is active at quite high temperature and at both neutral and acidic pH.

The purpose of the present invention is to provide variants of a lactase, such as a lactase derived from Bifidobacterium, e.g., Bifidobacterium bifidum, which have improved properties relative to the parent lactase, such as decreased galactose inhibition or increased galactose inhibition.

Variants with decreased or increased galactose inhibition may be beneficial since they allow for a fine-tuning of the galactose inhibition propensity of the lactase. A decreased galactose inhibition will allow the enzyme to retain more activity at higher levels of galactose and thus allow for degradation of lactose down to a lower final lactose level. An increased galactose inhibition may reduce or stop the activity of the enzyme when a certain decreased level of lactose has been obtained which may be advantageous in some commercial applications.

### SUMMARY OF THE INVENTION

The present inventors have for the first time produced protein engineered variants of a bacterial lactase, more specifically to a Bifidobacterium bifidum lactase which is widely used in the industry today.

The present inventors have found lactase variants having decreased or increased galactose inhibition compared to the parent lactase. The decreased or increased galactose inhibition has been determined by a shift in ratio of activity as measured by the galactose inhibition assay as disclosed herein.

Variants with decreased or increased galactose inhibition may be beneficial since they allow for a fine-tuning of the galactose inhibition propensity of the lactase. A decreased galactose inhibition will allow the enzyme to retain more activity at higher levels of galactose and thus allow for degradation of lactose down to a lower final lactose level. An increased galactose inhibition may reduce or stop the activity of the enzyme when a certain decreased level of lactose has been obtained which may be advantageous in some commercial applications.

The present invention is therefore directed to lactase variant having an amino acid sequence which is at least 80% identical to SEQ ID NO: 1 which comprises an alteration, preferably a substitution, at one or more positions corresponding to positions 2, 3, 4, 5, 6, 7, 9, 10, 11, 12, 13, 14, 15, 16, 17, 21, 22, 23, 24, 25, 26, 27, 28, 29, 31, 32, 33, 34, 35, 36, 37, 38, 39, 40, 41, 42, 43, 44, 45, 47, 48, 49, 51, 54, 55, 57, 58, 59, 60, 61, 62, 63, 64, 66, 67, 68, 70, 71, 73, 74, 75, 76, 77, 78, 79, 80, 81, 83, 85, 86, 87, 88, 89, 90, 91, 92, 93, 94, 95, 96, 97, 98, 100, 101, 102, 104, 105, 106, 107, 108, 109, 110, 111, 112, 113, 114, 115, 116, 117, 118, 119, 120, 121, 122, 123, 124, 125, 126, 128, 129, 130, 131, 132, 133, 135, 136, 137, 138, 139, 140, 141, 142, 146, 148, 150, 151, 153, 154, 155, 157, 158, 159, 160, 161, 162, 164, 165, 166, 167, 168, 169, 170, 171, 172, 173, 174, 175, 176, 177, 178, 179, 180, 181, 182, 183, 184, 185, 186, 187, 188, 189, 190, 191, 193, 194, 195, 196, 197, 198, 199, 201, 202, 203, 204, 206, 207, 208, 209, 210, 211, 212, 213, 214, 215, 216, 217, 218, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 254, 255, 256, 257, 258, 259, 260, 261, 262, 263, 265, 267, 268, 269, 271, 272, 273, 274, 275, 277, 278, 279, 280, 281, 282, 283, 284, 285, 286, 287, 288, 291, 292, 293, 294, 295, 296, 297, 298, 299, 301, 302, 303, 304, 305, 306, 307, 308, 310, 311, 312, 314, 315, 316, 317, 318, 320, 321, 322, 324, 325, 326, 328, 329, 331, 335, 337, 338, 340, 341, 342, 343, 344, 345, 346, 347, 348, 349, 350, 351, 352, 353, 354, 355, 357, 359, 360, 362, 363, 364, 365, 366, 367, 368, 369, 370, 371, 372, 373, 374, 375, 376, 377, 378, 379, 380, 381, 383, 386, 387, 388, 389, 390, 391, 392, 393, 394, 395, 396, 398, 399, 400, 401, 402, 403, 404, 406, 407, 408, 409, 410, 411, 412, 413, 414, 415, 416, 417, 418, 419, 421, 422, 423, 424, 425, 426, 427, 428, 429, 430, 431, 432, 433, 434, 435, 436, 437, 438, 439, 440, 441, 442, 443, 444, 445, 446, 448, 449, 450, 451, 452, 453, 454, 455, 456, 457, 459, 461, 462, 463, 464, 465, 466, 467, 469, 470, 471, 472, 473, 474, 475, 476, 477, 478, 479, 480, 485, 487, 488, 491, 492, 493, 494, 495, 496, 497, 498, 499, 500, 501, 502, 503, 504, 505, 506, 507, 508, 509, 510, 511, 512, 514, 516, 517, 518, 519, 520, 521, 524, 525, 526, 527, 528, 529, 530, 531, 534, 535, 537, 538, 539, 540, 542, 543, 545, 546, 547, 548, 549, 550, 551, 553, 554, 555, 556, 557, 558, 559, 560, 561, 563, 564, 565, 567, 570, 571, 572, 573, 574, 575, 576, 577, 578, 579, 580, 581, 582, 583, 584, 585, 586, 587, 588, 589, 590, 591, 592, 593, 594, 595, 597, 598, 600, 604, 605, 606, 608, 609, 610, 611, 612, 613, 631, 633, 634, 635, 636, 637, 638, 639, 640, 641, 642, 643, 644, 645, 646, 647, 648, 649, 650, 651, 652, 653, 654, 655, 656, 657, 659, 661, 662, 669, 670, 672, 673, 674, 675, 676, 677, 678, 679, 680, 681, 682, 683, 684, 685, 686, 687, 688, 689, 690, 693, 694, 695, 696, 697, 698, 700, 701, 703, 704, 705, 706, 708, 709, 710, 711, 712, 713, 714, 715, 718, 720, 721, 723, 724, 725, 726, 727, 728, 729, 730, 731, 732, 733, 734, 735, 736, 737, 738, 739, 740, 741, 742, 743, 744, 745, 746, 747, 748, 750, 751, 752, 753, 755, 756, 757, 758, 759, 760, 761, 762, 765, 766, 768, 769, 770, 771, 772, 773, 774, 775, 776, 777, 778, 779, 780, 781, 782, 783, 784, 785, 786, 787, 788, 789, 790, 791, 792, 793, 794, 795, 796, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 840, 841, 842, 843, 844, 845, 846, 847, 848, 849, 850, 851, 852, 853, 854, 855, 857, 858, 859, 860, 861, 862, 863, 864, 865, 866, 870, 871, 872, 873, 874, 875, 877, 879, 880, 881, 882, 883, 884, 885, 886, 887, 888, 889, 893, 894, 895, 896, 897, 898, 899, 900, 901, 902, 903, 904, 905, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 973, 974, 975, 976, 977, 978, 979, 980, 981, 984, 985, 986, 987, 988, 989, 990, 991, 992, 993, 994, 995, 996, 997, 998, 999, 1000, 1002, 1003, 1004, 1005, 1006, 1007, 1008, 1009, 1010, 1011, 1012, 1013, 1014, 1015, 1016, 1017, 1018, 1021, 1022, 1023, 1024, 1026, 1027, 1028, 1029, 1030, 1031, 1033, 1034, 1035, 1036, 1037, 1038, 1039, 1040, 1041, 1042, 1043, 1044, 1045, 1046, 1047, 1048, 1050, 1051, 1052, 1053, 1055, 1057, 1058, 1059, 1060, 1062, 1063, 1064, 1065, 1066, 1067, 1068, 1070, 1071, 1072, 1073, 1074, 1075, 1076, 1077, 1078, 1079, 1081, 1082, 1083, 1084, 1085, 1086, 1087, 1088, 1089, 1090, 1091, 1092, 1093, 1094, 1095, 1096, 1097, 1098, 1099, 1100, 1101, 1102, 1103, 1104, 1105, 1106, 1107, 1108, 1109, 1111, 1113, 1114, 1115, 1116, 1117, 1118, 1119, 1120, 1121, 1122, 1123, 1124, 1125, 1126, 1127, 1128, 1129, 1130, 1131, 1132, 1133, 1134, 1135, 1136, 1137, 1138, 1139, 1140, 1141, 1142, 1143, 1144, 1145, 1146, 1147, 1148, 1149, 1150, 1151, 1152, 1153, 1154, 1155, 1157, 1158, 1159, 1161, 1162, 1166, 1167, 1168, 1169, 1170, 1171, 1172, 1173, 1174, 1175, 1177, 1178, 1179, 1180, 1181, 1182, 1184, 1185, 1186, 1188, 1189, 1190, 1191, 1192, 1193, 1194, 1195, 1197, 1198, 1199, 1202, 1203, 1204, 1205, 1208, 1209, 1210, 1211, 1213, 1214, 1215, 1217, 1218, 1219, 1220, 1221, 1222, 1223, 1224, 1225, 1226, 1227, 1228, 1230, 1231, 1232, 1233, 1234, 1235, 1236, 1237, 1238, 1239, 1240, 1241, 1242, 1243, 1244, 1246, 1247, 1248, 1249, 1250, 1251, 1252, 1253, 1254, 1255, 1256, 1257, 1258, 1259, 1261, 1262, 1263, 1264, 1265, 1266, 1267, 1269, 1270, 1271, 1272, 1273, 1274, 1275, 1276, 1277, 1278, 1279, 1280, 1281, 1282, 1283, 1284, 1285, 1286, 1287, 1288, 1289, 1290, 1291, 1292, 1293, 1294, 1295, 1296, 1297, 1298, 1301, 1302, 1303 or 1304 of the polypeptide of SEQ ID NO: 1.

The invention is also directed to lactase variant having an amino acid sequence which is at least 80% identical to SEQ ID NO: 1 which comprises one or more of the following substitutions, wherein each position corresponds to a position in SEQ ID NO: 1: V1D, V1F, V1H, V1K, V1L, V1R, E2D, E2G, E2Q, E2V, D3A, D3H, D3I, D3N, D3S, D3V, D3W, A4C, A4G, A4H, A4I, A4L, A4M, A4P, T5A, T5D, T5F, T5K, T5R, T5S, T5V, R6A, R6D, R6F, R6G, R6H, R6L, R6M, R6P, R6S, R6W, S7A, S7D, S7I, S7L, S7N, S7P, S7T, S7W, S9G, S9H, S9P, S9R, S9W, T10G, T10K, T10L, T10P, T10R, T10S, T10W, T11L, T11P, Q12L, Q12R, Q12V, Q12Y, M13C, M13D, M13E, M13F, M13H, M13K, M13R, M13W, S14A, S14G, S14H, S14L, S14T, S14V, S14Y, S15C, S15F, S15I, S15K, S15L, S15P, S15R, S15T, S15V, S15W, S15Y, T16A, T16C, T16I, T16N, T16S, P17A, P17C, P17D, P17E, P17I, P17L, P17N, P17R, P17S, P17T, V19A, V19F, V19G, V19I, V19K, V19L, V19N, V19S, V19W, V20C, V20F, V20G, V20I, V20K, V20L, V20M, V20N, V20P, V20Q, V20R, V20T, V20W, Y21A, Y21C, Y21D, Y21F, Y21G, Y21H, Y21M, Y21P, Y21R, Y21T, S22A, S22E, S22F, S22G, S22L, S22M, S22N, S22R, S22T, S22W, S23A, S23C, S23D, S23L, S23M, S23R, A24F, A24G, A24L, A24R, A24T, A24W, V25D, V25E, V25F, V25G, V25H, V25K, V25L, V25M, V25Q, V25R, V25S, V25T, V25W, D26C, D26I, D26L, D26M, D26T, D26V, S27A, S27C, S27F, S27G, S27H, S27P, S27Y, K28C, K28G, K28I, K28L, K28R, K28S, K28V, K28W, Q29D, Q29F, Q29G, Q29L, Q29M, Q29R, Q29S, Q29V, Q29W, N30A, N30G, N30H, N30M, N30P, N30V, N30W, N30Y, R31E, R31G, R31I, R31M, R31V, T32M, T32Q, T32R, T32S, S33C, S33E, S33H, S33K, S33N, S33Q, S33R, S33V, D34C, D34E, D34F, D34G, D34H, D34L, D34S, D34W, D34Y, F35A, F35C, F35E, F35G, F35K, F35N, F35T, F35V, D36H, D36Q, A37N, A37Q, N38C, N38G, N38S, W39G, W39S, K40C, K40D, K40F, K40G, K40I, K40M, K40N, K40P, K40W, F41A, F41C, F41G, F41I, F41Q, F41S, F41Y, M42E, M42N, M42T, L43A, L43C, L43G, L43I, L43S, L43T, L43V, S44C, S44M, S44N, S44Y, D45A, D45L, D45P, D45V, V47K, V47R, Q48S, A49C, A49D, A49H, A49R, A49S, A49T, A49V, D51G, D51I, D51K, D51M, D51P, D51V, A53C, A53G, A53L, A53R, A53S, A53V, A53W, F54M, F54S, D55C, D55F, D55G, D55H, D55M, D55N, D55P, D55S, D55V, S57A, S57C, S57E, S57G, A58D, A58G, A58I, A58M, A58N, A58Q, A58R, A58T, W59D, W59I, W59K, W59L, W59N, W59P, W59V, Q60A, Q60C, Q60E, Q60F, Q60G, Q60K, Q60L, Q60M, Q60R, Q60S, Q60V, Q60Y, Q61K, Q61P, Q61S, V62G, V62N, V62S, V62T, V62W, D63G, D63L, D63P, D63S, D63V, L64E, L64G, H66C, H66L, H66R, H66T, H66W, H66Y, D67E, Y68P, Y68V, I70A, I70H, I70K, I70P, I70R, T71C, T71E, T71G, T71H, T71K, T71L, T71N, T71P, T71Q, T71R, T71S, K73A, K73D, K73F, K73G, K73Q, K73V, Y74G, Y74K, Y74T, Y74W, S75G, S75L, S75Q, S75R, S75V, Q76G, Q76I, Q76K, Q76M, Q76P, Q76S, Q76V, Q76Y, S77C, S77D, S77E, S77F, S77G, S77H, S77I, S77K, S77L, S77M, S77R, S77T, S77V, S77W, S77Y, N78C, N78E, N78F, N78K, N78Q, N78R, N78S, N78T, E79H, E79Q, E79S, E79T, E79W, A80K, E81A, E81Q, A83E, A83T, L85A, L85C, L85D, L85F, L85M, L85N, L85S, L85V, L85W, P86E, P86G, P86H, P86N, P86Q, P86R, P86V, P86W, P86Y, G87A, G87D, G87E, G87N, G87Q, G88A, G88F, G88I, G88M, G88Q, G88S, T89C, T89G, T89H, T89K, T89L, T89M, T89N, T89P, T89W, T89Y, G90A, G90C, G90D, G90L, G90S, G90T, G90V, W91E, W91L, W91P, W91Q, W91R, W91S, W91Y, Y92F, Y92H, Y92I, Y92M, Y92N, Y92S, Y92T, Y92V, Y92W, R93A, R93D, R93F, R93H, R93I, R93L, R93N, R93T, R93V, R93Y, K94A, K94C, K94G, K94R, K94S, K94T, K94V, S95A, S95C, S95D, S95E, S95G, S95I, S95L, S95Q, S95R, F96A, F96C, F96I, F96K, F96L, F96M, F96P, F96S, F96V, F96W, T97F, T97S, T97V, I98C, I98H, I98S, I98W, R100T, D101A, D101P, D101V, L102A, L102G, L102M, L102P, L102S, G104C, K105D, K105Q, K105R, K105W, K105Y, R106K, R106P, R106V, R106W, I107A, I107F, I107G, I107Q, I107S, A108E, A108S, A108V, I109M, I109T, N110A, N110F, N110S, N110T, N110V, N110W, F111A, F111C, F111L, F111Q, F111V, D112A, D112F, D112G, D112T, G113A, G113S, V114F, V114G, V114M, V114R, Y115E, M116A, M116C, M116D, M116W, M116Y, N117K, N117R, N117T, N117W, A118K, A118P, A118Y, T119A, T119G, T119L, V120A, V120K, W121C, W121D, W121R, W121T, W121V, W121Y, F122A, F122M, F122S, F122Y, N123P, G124E, G124M, G124Q, G124R, V125D, V125E, V125I, K126E, K126V, G128A, G128D, T129E, T129V, H130A, H130C, H130Q, H130S, H130T, P131K, P131L, P131S, Y132C, Y132E, Y132S, G133E, S135E, S135P, S135V, P136R, P136Y, F137A, F137C, F137D, F137G, F137L, F137P, S138A, S138D, S138G, S138H, S138L, S138M, S138R, S138V, F139A, F139E, F139Q, F139W, D140G, D140L, D140V, L141G, L141T, T142E, T142S, T142V, K146A, G148H, G148K, G148T, G149H, G149I, G149M, G149Q, G149Y, E150A, E150C, E150G, E150L, E150N, E150R, N151L, I153A, I153Y, V154E, V154I, V154K, V154L, V154M, V154S, V155F, V157A, V157G, V157L, V157P, V157Q, V157S, E158G, E158H, E158K, E158Q, E158V, N159D, N159H, N159S, N159T, R160G, L161E, L161K, L161M, L161S, L161W, P162F, P162G, P162N, P162T, P162W, S164A, R165H, W166S, Y167A, Y167C, S168C, G169A, G169C, G169D, G169S, S170L, S170Q, G171C, G171F, G171T, I172G, I172K, I172P, I172Q, Y173A, Y173H, Y173M, Y173P, Y173S, Y173W, R174E, R174K, D175E, D175Y, V176E, V176K, V176T, T177A, T177C, T177E, T177K, T177L, L178I, L178Q, L178W, T179A, T179C, T179D, T179H, T179I, T179K, T179L, T179N, T179P, T179S, V180A, V180C, V180D, V180E, V180G, V180M, T181A, T181D, T181F, T181K, T181R, D182F, D182L, D182S, G183W, V184F, V184H, V184P, V184Q, V184R, V184S, V184W, H185G, H185L, H185R, V186A, V186E, V186G, V186N, G187A, G187D, G187H, N188E, N188R, N188S, N188V, N188W, N189A, N189E, G190C, G190F, G190H, G190Q, G190V, V191C, V191T, V191W, V191Y, I193N, I193Q, I193T, I193V, K194A, K194I, K194L, K194R, T195A, T195E, T195M, T195S, T195W, P196A, P196H, P196I, P196M, P196S, P196W, S197A, S197C, S197E, S197K, S197L, S197P, L198E, L198F, L198H, L198I, L198K, L198R, L198V, L198W, A199E, A199F, A199K, A199P, A199R, A199T, Q201C, Q201E, Q201I, Q201K, Q201M, Q201V, N202A, N202D, N202F, N202G, N202K, N202L, N202M, N202Q, N202R, N202S, N202T, N202W, G203C, G203K, G203M, G203Q, G203R, G203S, G203V, G203W, G203Y, G204A, G204C, G204D, G204K, G204R, G204S, G204Y, N205E, N205G, N205H, N205L, N205P, N205W, N205Y, V206A, V206C, V206D, V206F, V206G, V206I, V206K, V206Q, V206R, V206S, V206T, T207A, T207C, T207G, T207I, T207K, T207L, T207M, T207N, T207Q, T207R, T207W, M208A, M208S, M208T, N209C, N209D, N209G, N209K, N209L, N209Q, N209R, N209V, L210A, L210C, L210F, L210G, L210H, L210I, L210Q, L210R, L210S, L210T, L210V, T211A, T211D, T211E, T211F, T211K, T211N, T211Q, T211R, T211S, T212A, T212C, T212E, T212F, T212G, T212H, T212K, T212L, T212M, T212S, T212W, K213A, K213C, K213D, K213F, K213I, K213L, K213M, K213N, K213Q, K213R, K213S, K213T, K213V, K213Y, V214A, V214C, V214T, V214W, A215D, A215E, A215F, A215I, A215K, A215L, A215Q, A215R, A215S, A215V, N216D, N216T, N216V, D217F, D217G, D217L, D217M, D217T, D217V, T218D, T218G, T218H, K219A, K219C, K219F, K219H, K219M, A220C, A220G, A220I, A220L, A220M, A220T, A220V, A220W, A221C, A221D, A221E, A221L, A221N, A221R, A221V, A221Y, A222D, A222I, A222L, A222P, A222R, A222W, A222Y, N223A, N223E, N223F, N223G, N223K, N223L, N223M, N223R, N223S, N223T, N223V, N223W, I224G, I224Q, T225A, T225G, T225L, L226C, L226M, L226Q, K227T, Q228N, Q228R, T229A, T229C, T229D, T229G, T229H, T229M, T229N, T229Q, T229R, T229V, V230F, V230L, V230M, V230Q, V230R, V230S, F231A, F231E, F231G, F231I, F231K, F231L, F231Q, F231S, F231V, F231W, F231Y, P232G, P232H, P232L, P232M, P232R, P232S, P232T, P232V, P232W, P232Y, K233A, K233C, K233E, K233F, K233G, K233L, K233P, K233R, K233S, K233V, K233W, K233Y, G234A, G234C, G234D, G234E, G234K, G234L, G234Q, G234R, G234V, G234W, G234Y, G235C, G235F, G235H, G235I, G235K, G235M, G235Q, G235R, G235T, G235W, G235Y, K236A, K236D, K236E, K236G, K236L, K236M, K236P, K236R, K236S, K236T, K236W, K236Y, T237D, T237F, T237I, T237K, T237M, T237Q, T237R, T237S, T237V, T237Y, D238A, D238E, D238F, D238G, D238H, D238I, D238K, D238L, D238M, D238N, D238P, D238Q, D238R, A239C, A239E, A239G, A239I, A239K, A239T, A240C, A240E, A240L, A240P, A240Q, A240T, A240V, A240W, A240Y, I241T, G242K, G242L, G242M, G242P, G242T, G242Y, T243I, T243M, T243R, T243V, V244A, V244E, V244G, V244L, V244R, T245E, T245G, T245L, T245M, T245N, T245Q, T245R, T245S, T246D, T246E, T246G, T246K, T246V, A247D, A247E, A247K, A247N, A247P, A247Q, A247R, A247S, A247V, A247W, S248A, S248E, S248F, S248H, S248I, S248L, S248Q, S248T, S248Y, K249A, K249D, K249G, K249H, K249I, K249L, K249M, K249N, K249P, K249Q, K249S, K249T, K249V, K249Y, S250H, S250M, S250W, I251F, I251L, I251V, I251W, I251Y, A252C, A252E, A252F, A252H, A252I, A252P, A252R, A252S, A252W, A252Y, G254D, G254F, G254I, G254L, G254M, G254Q, G254R, G254W, A255C, A255F, A255K, A255L, A255M, A255S, A255T, A255W, A255Y, S256A, S256C, S256F, S256G, S256K, S256L, S256M, S256N, S256Q, S256R, S256V, S256W, S256Y, A257D, A257G, A257I, A257N, A257T, A257V, D258A, D258L, D258M, D258W, V259E, V259L, V259S, V259T, T260A, T260D, T260G, T260I, T260K, T260V, S261A, S261D, S261H, S261R, S261W, S261Y, T262D, T262E, T262F, T262G, T262H, T262L, T262P, T262W, I263A, I263C, I263G, I263L, I263S, I263V, T264F, T264G, T264K, T264L, T264M, T264P, T264Q, T264R, T264S, T264Y, A265G, A265I, A265K, A265R, A265S, A265V, A266D, A266E, A266G, A266K, A266L, A266M, A266P, A266Q, A266S, S267A, S267D, S267K, S267M, S267N, S267P, S267Q, S267R, S267V, P268F, P268G, P268M, P268R, P268V, P268W, P268Y, K269G, K269R, K269V, K269Y, L270D, L270M, L270N, L270R, L270V, W271T, S272E, S272G, S272K, S272L, S272N, S272T, S272W, I273K, I273L, I273P, I273R, I273S, I273W, K274D, K274P, K274Q, K274R, N275K, N275M, N275V, N275W, N277F, N277K, N277R, L278A, L278G, L278H, L278I, L278K, L278M, L278P, L278Q, L278R, L278S, L278V, Y279M, Y279T, Y279W, T280A, T280D, T280E, T280F, T280H, T280M, T280Q, V281A, V281I, V281L, V281Q, R282E, R282F, R282H, R282I, R282K, R282N, R282S, R282T, R282V, R282W, T283M, T283R, T283V, E284A, E284D, E284F, E284H, E284L, E284M, E284N, E284Q, E284R, E284Y, V285H, V285I, V285T, L286A, L286C, L286D, L286F, L286N, L286R, L286T, L286W, L286Y, N287I, N287L, G288F, G288L, G288S, V291C, V291D, V291F, V291G, V291H, V291L, V291P, V291S, V291T, V291Y, L292A, L292D, L292E, L292H, L292Q, L292S, L292V, D293C, D293E, D293F, D293I, D293S, D293V, D293W, T294C, T294G, T294M, T294Q, T294S, Y295F, Y295G, Y295I, Y295L, Y295R, Y295S, Y295W, D296F, D296H, D296K, D296L, D296N, D296R, D296V, T297G, T297I, T297Q, T297R, T297S, E298G, E298I, E298L, E298M, E298R, E298T, Y299F, Y299S, F301C, F301L, R302E, R302I, R302K, R302M, R302N, R302T, W303A, W303C, W303D, W303F, W303S, W303T, T304D, T304E, T304I, T304K, T304P, T304S, T304W, G305E, G305F, G305L, G305M, G305N, G305P, G305T, G305W, F306D, F306K, F306L, F306S, D307A, D307E, D307F, D307G, D307L, D307M, D307N, D307Q, D307S, D307V, D307W, A308C, A308E, A308G, A308I, A308M, A308Y, T309C, T309D, T309E, T309I, T309K, T309M, T309S, T309V, S310A, S310C, S310G, S310H, S310I, S310L, S310M, S310N, S310R, S310V, G311E, G311F, G311L, G311Q, G311R, G311V, F312H, F312L, F312R, F312V, L314A, L314C, L314T, L314V, L314Y, N315S, G316A, G316C, G316N, G316R, G316S, G316W, G316Y, E317D, E317H, E317I, E317K, E317V, E317W, E317Y, K318C, K318E, K318F, K318G, K318H, K318L, K318M, K318N, K318P, K318Q, K318V, K320C, K320E, K320F, K320I, K320L, K320R, K320T, K320V, K320W, L321C, L321G, L321H, L321M, L321V, L321Y, K322C, K322F, K322I, K322N, K322P, K322Q, K322R, K322S, V324E, V324G, V324T, S325D, S325G, S325T, M326E, M326G, M326S, M326T, M326V, M326Y, H328C, H328D, H328F, H328G, H3281, H328L, H328M, H328R, H328T, D329S, D329T, G331V, A335C, A335G, A335L, A337D, A337E, A337G, A337L, A337Q, N338P, N338R, N338V, R340A, R340C, R340L, A341M, A341S, I342A, I342G, I342K, I342P, E343A, E343H, E343N, E343R, E343S, E343T, E343Y, R344G, R344L, R344Y, Q345F, Q345G, Q345K, Q345N, Q345S, V346A, V346C, V346F, V346I, V346L, V346S, E347A, E347C, E347D, E347F, E347I, E347R, E347S, I348A, I348D, I348G, I348M, I348Q, I348R, L349A, L349E, L349Q, L349S, L349V, Q350G, Q350N, Q350W, K351N, K351R, K351V, K351W, M352G, M352L, M352T, M352W, G353K, V354E, V354G, V354S, V354W, N355H, N355M, N355R, N355W, I357T, T359E, T359L, T360V, N362G, N362S, N362T, P363A, P363D, P363I, P363L, P363M, P363S, P363V, A364C, A364E, A364F, A364G, A364I, A364M, A364P, A364V, A364W, A365C, A365E, A365I, A365P, A365V, A365W, K366A, K366D, K366E, K366I, K366L, K366M, K366P, K366S, K366V, A367C, A367I, A367N, A367Q, L368A, L368E, L368Q, L368S, L368V, L368Y, I369A, I369D, I369E, I369G, I369K, I369M, I369R, I369V, I369W, D370C, D370L, D370Q, D370R, D370S, D370T, V371D, V371F, V371G, V371I, V371L, V371Q, V371S, C372P, N373G, N373L, N373R, E374K, E374L, E374R, K375D, K375I, K375N, K375Q, K375S, G376A, G376S, V377A, V377M, V377T, L378I, L378P, L378W, L378Y, V379A, V379C, V379M, V379N, V380M, V380P, V380S, E381A, E381C, E381G, E381L, E381Q, E381T, V383A, V383K, V383L, M386G, M386N, M386Q, M386S, M386V, W387H, W387L, N388A, N388E, N388L, N388R, R389A, R389C, R389E, R389K, R389M, R389N, R389Q, R389S, R389T, R389V, S390C, S390D, S390G, S390H, S390N, S390P, S390Q, S390T, S390V, K391E, N392D, G393A, G393E, G393N, G393R, G393S, G393V, N394L, T395A, T395C, T395F, T395H, T395I, T395M, T395N, T395Q, T395S, T395W, E396K, E396L, E396M, E396V, E396W, Y398M, Y398N, G399S, K400A, K400C, K400D, K400E, K400M, K400N, K400P, K400Q, K400S, K400T, K400V, W401F, W401H, W401K, W401L, W401M, W401R, F402T, F402W, F402Y, G403A, G403D, G403H, G403K, G403P, G403Q, G403S, G403T, G403V, G403Y, Q404F, Q404H, Q404L, Q404M, Q404P, Q404R, Q404S, Q404V, A405C, A405E, A405H, A405K, A405P, A405R, A405V, I406C, I406D, I406N, A407C, A407G, A407M, A407Q, A407S, A407T, A407W, G408D, G408I, G408M, G408N, G408W, D409N, D409W, N410C, N410R, N410Y, A411E, A411N, A411R, A411S, A411V, V412M, V412S, L413D, L413E, L413F, L413I, L413P, L413T, G414A, G414C, G414M, G414N, G414R, G414T, G414W, G415A, G415Q, G415R, D416I, D416M, D416R, D416T, D416Y, K417C, K417F, K417G, K417R, K417T, D418L, D418P, D418R, D418Y, E419M, E419R, E419W, T420E, T420F, T420G, T420K, T420R, T420V, W421L, W421Q, W421S, A422P, A422T, A422V, K423D, K423L, K423M, K423R, F424C, F424L, F424N, F424T, D425E, L426C, L426M, L426Q, T427D, T427F, T427G, T427K, T427M, T427P, T427Q, T427R, T427S, T427W, S428F, S428K, S428W, T429D, T429P, I430C, I430D, I430E, I430L, I430M, I430Q, I430S, I430T, I430W, N431D, N431E, N431G, N431L, N431M, N431R, N431V, N431Y, R432A, R432E, R432F, R432G, R432N, R432Q, R432V, R432Y, D433C, D433G, D433H, D433I, D433P, D433Q, D433W, R434L, R434M, R434N, R434P, R434S, R434T, R434V, N435E, N435F, N435H, N435K, N435L, N435M, N435R, N435V, N435W, A436C, A436D, A436E, A436G, A436I, A436L, A436M, A436Q, A436S, P437A, P437D, P437K, P437L, P437Q, P437R, P437S, P437V, P437W, S438G, V439C, V439E, V439G, V439I, V439K, V439Q, V439T, V439Y, I440C, I440D, I440F, I440K, I440P, I440R, I440S, I440T, I440V, I440W, M441A, M441E, M441G, M441Q, M441R, M441T, M441V, W442E, W442G, W442M, W442P, W442Q, W442R, S443C, S443D, S443G, S443M, S443Q, S443Y, L444C, L444D, L444E, L444F, L444G, L444H, L444K, L444Q, L444V, L444W, G445A, G445C, G445V, N446D, N446T, M448A, M448C, M448D, M448E, M448I, M448L, M448P, M448Q, M448S, M448V, M448W, M449D, M449E, M449F, M449T, M449V, M449W, M449Y, E450F, E450S, E450T, E450V, E450W, G451C, G451F, G451L, G451P, G451V, G451W, I452G, I452K, I452L, I452M, I452Q, I452S, I452V, S453C, S453F, S453G, S453H, S453L, S453M, S453N, S453P, S453Q, S453R, S453V, G454L, G454W, S455A, S455E, S455K, S455M, S455P, S455R, S455V, S455W, V456A, V456D, V456E, V456F, V456K, V456L, V456W, S457E, S457H, S457K, S457L, S457M, S457P, S457Q, S457T, S457V, G458A, G458C, G458F, G458L, G458P, G458Q, G458S, G458V, G458W, F459A, F459C, F459E, F459G, F459N, F459R, F459S, F459T, F459W, P460C, P460M, P460Q, P460W, P460Y, A461D, A461G, A461M, A461N, A461Q, A461S, A461V, A461Y, T462C, T462E, T462F, T462L, T462M, T462S, S463G, S463K, S463Q, S463R, S463T, S463V, A464E, A464H, A464L, A464M, A464P, A464V, A464W, K465C, K465F, K465G, K465L, K465Q, K465R, K465V, K465W, K465Y, L466A, L466C, L466D, L466E, L466F, L466G, L466M, L466P, L466Q, L466S, L466V, L466Y, V467A, V467C, V467D, V467E, V467G, V467T, V467W, A468D, A468E, A468F, A468K, A468L, A468P, A468S, A468V, A468W, W469A, W469C, W469D, W469G, W469L, W469M, W469R, W469V, W469Y, T470E, T470L, T470M, T470Q, K471F, K471G, K471Q, K471W, K471Y, A472G, A472Y, A473E, A473M, A473P, D474A, D474C, D474E, D474K, D474M, D474R, D474W, S475E, S475F, S475Q, S475T, S475V, T476C, T476L, T476S, R477A, R477C, R477G, R477L, P478A, P478D, P478G, P478L, P478V, M479G, M479I, M479R, M479W, T480C, T480G, T480Q, K485E, K485R, K487A, K487C, K487F, K487G, K487N, K487S, K487W, A488C, A488G, A488H, A488L, A488N, A488S, A488V, N491A, N491E, N491W, E492A, E492W, S493E, S493G, S493H, S493L, S493M, S493Q, S493V, N494A, N494I, N494M, N494R, N494V, T495K, T495R, T495V, T495W, M496A, M496F, M496T, G497D, D498A, D498C, D498M, D498S, N499K, N499R, N499T, N499Y, L500A, L500E, L500N, L500V, T501C, T501G, T501M, A502L, A502Q, N503A, N503E, N503M, N503S, G504H, G504K, G504P, G505A, G505D, G505E, G505H, G505L, G505N, G505R, G505S, G505V, V506C, V506D, V506E, V506G, V506I, V506L, V506P, V506R, V506S, V506T, V506W, V507A, V507F, V507G, V507L, V507N, V507P, V507R, V507S, V507T, G508C, G508E, T509A, T509D, T509E, T509I, T509K, T509M, T509Q, T509S, T509V, T509Y, N510A, N510F, N510I, N510Q, Y511A, Y511K, S512C, S512E, S512F, S512G, S512I, S512M, S512Q, S512T, S512V, S512Y, D513C, D513G, D513K, D513L, D513M, D513P, D513Q, D513R, D513W, G514F, G514L, G514N, G514P, G514R, A515C, A515D, A515E, A515F, A515G, A515K, A515R, A515S, N516C, N516E, N516G, N516I, N516M, N516Q, N516S, N516T, N516V, Y517G, Y517I, Y517N, D518Q, D518Y, K519C, K519E, K519F, K519G, K519I, K519L, K519M, K519N, K519Q, K519S, K519T, I520A, I520C, I520G, I520H, I520M, I520N, I520Q, I520S, I520T, I520W, I520Y, R521A, R521C, R521K, R521N, R521Q, R521V, T522G, T522I, T522N, T523A, T523M, H524R, H524V, P525D, P525G, P525R, P525T, P525V, S526G, S526I, S526W, W527A, W527E, W527G, W527H, W527N, W527R, W527S, W527V, A528C, A528E, A528G, A528I, A528L, I529F, I529G, I529L, I529Y, Y530A, Y530M, G531E, G531S, G531T, T534A, T534I, T534Q, A535E, A535G, A535I, A535M, A537D, A537M, A537P, A537S, I538H, I538M, N539G, N539W, S540E, S540G, S540M, G542E, G542Q, G542S, G542T, I543V, I543W, N545G, N545I, N545Q, N545S, R546A, R546C, R546L, R546P, R546S, T547A, T547D, T547H, T547K, T547N, T547S, T548D, T548E, T548F, T548K, T548L, T548P, T548W, G549D, G549F, G549P, G549W, G550Q, G550R, G550S, A551D, A551I, A551Q, S553C, S553F, S553N, S553P, S553R, S553T, S553V, S554F, S554N, S554R, S554T, S554V, S554W, D555E, D555P, D555S, K556A, K556C, K556R, K556W, Q557F, Q557R, Q557S, L558E, L558H, L558I, L558P, T559A, T559G, T559I, T559Q, T559V, T559Y, S560P, S560V, Y561R, N563R, N563S, S564A, S564C, S564F, S564W, A565C, A565D, A565E, A565F, A565G, A565I, A565L, A565M, A565R, A565S, A565T, A565V, A565W, G567N, G567Q, G567V, A570G, A570K, A570L, A570M, A570S, A570T, A570W, V571W, A572S, A572W, S573G, S573K, S573Y, S574K, S574Q, S574V, S574W, A575D, A575M, A575V, W576A, W576F, W576V, W576Y, Y577G, Y577I, Y577L, Y577R, D578E, D578M, D578N, D578T, V579E, V579G, V579L, V579T, V580A, V580D, V580E, V580K, V580L, V580S, Q581F, Q581G, Q581P, Q581R, Q581S, Q581T, Q581Y, R582A, R582D, R582G, R582I, R582L, R582Y, D583V, D583W, F584E, F584I, F584W, V585I, V585M, V585Q, A586C, A586D, A586H, A586K, G587A, G587C, T588C, T588D, T588G, T588I, T588L, T588M, T588P, Y589A, Y589I, Y589Q, Y589V, Y589W, V590A, V590H, V590I, W591F, T592C, T592L, T592Q, T592S, G593C, G5931, F594C, F594I, F594L, F594M, D595E, D595Q, D595S, L597C, L597D, L597E, L597T, G598N, P600A, P600E, P600G, P600S, N604E, N604S, G605R, T606S, G607A, G607Q, G607S, S608E, S608I, S608M, S608N, S608Q, S608T, S608V, G609A, G609H, G609L, G609N, G609R, G609S, A610D, A610F, A610M, A610T, V611K, G612N, G612T, G612V, S613A, W614A, W614F, W614P, P615L, P615S, P615V, S616A, S616W, N619A, N619I, N619L, N619S, S620G, Y621W, I624A, I624V, V625A, V625E, V625F, V625M, V625Y, T627F, T627G, T627K, T627Q, A628C, A628D, A628N, G629T, F630A, F630C, F630D, F630G, F630S, F630Y, P631A, P631D, P631G, P631H, P631S, P631V, P631Y, K632C, K632D, K632G, K632T, D633G, T634A, T634E, T634F, T634S, T634V, Y635R, Y636K, F637C, F637G, F637I, F637S, F637T, F637V, Y638A, Y638W, Q639D, Q639N, Q639R, S640C, S640D, Q641T, W642I, N643R, D644C, D644G, D644Y, D645S, D645V, V646C, V646L, V646N, V646R, V646S, H647G, H647V, T648C, L649V, H650E, H650F, H650R, I651F, I651T, I651V, L652C, L652D, L652V, L652W, P653Q, A654D, A654K, A654M, A654R, W655F, W655R, N656A, N656K, N656V, E657K, E657R, E657V, V659D, V659N, A661E, A661G, A661H, A661K, A661L, A661M, A661Q, A661W, K662H, K662S, K662V, K662W, K662Y, N667L, N667R, P669A, P669E, P669F, P669L, P669R, P669T, P669W, V670C, V672L, Y673E, Y673G, Y673I, Y673R, Y673S, Y673W, T674C, T674D, T674G, T674H, T674M, T674Q, D675A, D675E, D675P, D675Q, D675S, D675V, D675Y, A676C, A676E, A676G, A676K, A676L, A676P, A676S, A676W, A677E, A677G, A677L, A677R, A677T, A677V, A677Y, K678A, K678C, K678T, K678V, V679G, V679Q, V679S, V679T, V679Y, K680A, K680E, K680G, K680H, K680I, K680L, K680N, K680Q, K680S, K680V, K680W, L681E, L681F, L681G, L681M, L681S, L681T, Y682D, Y682E, Y682I, Y682M, Y682S, Y682V, Y682W, F683H, F683L, F683M, F683Q, F683R, F683W, T684A, T684D, T684G, T684L, T684R, T684S, T684V, P685A, P685E, P6851, P685L, P685W, K686A, K686E, K686I, K686M, K686T, K686V, G687A, G687K, G687N, G687P, G687Q, G687R, S688C, S688D, S688E, S688G, S688K, S688L, S688P, S688T, T689A, T689D, T689E, T689G, T689L, T689P, T689S, T689W, T689Y, E690D, E690M, E690P, E690T, E690V, K691E, K691H, K691N, K691P, K691R, K691S, R692G, R692H, R692I, R692L, R692P, R692S, R692T, R692V, R692W, L693A, L693M, L693P, I694L, I694W, G695C, G695K, G695L, G695R, G695W, E696A, E696L, E696R, K697A, K697E, K697G, K697R, K697V, K697W, S698C, S698D, S698E, S698I, S698L, S698M, S698P, S698Q, S698R, S698T, T700A, T700C, T700D, T700E, T700G, T700K, T700Y, K701A, K701D, K701E, K701G, K701H, K701L, K701M, K701P, K701S, K701W, T703E, T703I, T703K, T703W, T704M, T704R, T704Y, A705C, A705D, A705E, A705K, A705N, A705P, A705R, A705V, A705W, A706C, A706G, A706T, A706W, A706Y, Y708C, Y708F, Y708G, Y708K, Y708L, Y708P, Y708T, T709M, Y710C, Y710D, Y710E, Y710G, Y710M, Y710N, Y710T, Y710V, Y710W, Q711A, Q711D, Q711G, Q711L, Q711M, Q711T, Q711Y, V712G, V712M, V712P, V712Q, V712R, Y713A, Y713E, Y713G, Y713L, Y713Q, E714D, E714H, E714I, E714K, E714M, E714N, E714R, E714S, E714V, E714Y, G715K, A716C, A716G, A716L, A716M, A716P, A716R, A716V, D717C, D717G, D717L, D717S, K718A, K718L, K718Q, K718T, K718W, K718Y, D719E, D719L, D719P, D719S, D719T, D719V, S720A, S720M, S720R, S720Y, T721D, T721H, T721K, T721L, T721N, T721P, T721Q, T721S, T721V, T721W, T721Y, A722Q, A722T, H723G, H723P, K724A, K724G, K724R, N725A, N725D, N725I, M726A, M726D, M726E, M726K, M726Q, M726V, M726W, Y727L, Y727T, L728C, L728K, L728Q, L728S, L728T, L728W, T729A, T729E, T729M, W730A, N731A, N731E, N731Q, N731S, N731Y, V732G, V732P, V732R, V732W, P733A, P733R, P733W, W734D, W734G, W734R, W734V, A735Q, A735R, A735S, A735T, A735W, E736S, G737F, G737K, G737N, G737Y, T738G, T738L, T738S, I739E, I739K, I739M, I739W, I739Y, S740D, S740E, S740F, S740H, S740L, A741I, A741P, A741S, A741V, E742D, E742M, E742Q, E742V, A743C, A743E, A743H, A743I, A743L, Y744A, Y744E, Y744I, Y744K, Y744L, Y744R, D745C, D745F, D745N, D745R, E746A, E746C, E746K, E746T, N747E, N747F, N747G, N747P, N747R, N748S, R749H, R749M, R749S, R749T, L750G, L750M, L750P, L750Q, L750S, I751C, I751H, I751Q, I751S, P752A, P752C, P752H, P752L, P752S, P752V, P752Y, E753Q, G754H, G754I, G754P, S755C, S755I, S755T, T756E, T756N, T756P, T756Q, T756S, T756W, E757A, G758A, G758V, N759D, N759R, N759S, N759V, A760G, A760N, A760P, A760Q, S761A, S761K, S761Q, V762D, V762G, V762K, V762W, T765A, T765P, T765R, T765W, G766M, G766S, A768H, A769F, A769G, A769I, A769N, A769V, A769Y, K770H, L771A, L771I, K772A, K772C, K772P, K772V, K772W, A773C, A773G, A773H, A773M, A773R, A773S, A773V, D774A, D774R, A775L, A775V, A775W, D776I, D776L, D776R, D776S, D776V, R777D, R777E, R777G, R777H, R777P, R777S, R777T, K778A, K778C, K778F, K778G, K778L, K778N, K778R, T779C, T779I, I780V, T781A, T781C, T781E, T781F, T781G, T781M, T781P, T781R, T781Y, A782C, A782E, A782K, A782N, A782P, A782Q, A782Y, D783A, D783C, D783E, D783R, G784A, G784F, G784L, G784S, G784T, K785C, K785I, K785S, K785V, K785W, K785Y, D786S, D786V, L787D, L787K, L787P, L787T, L787Y, S788A, S788G, S788I, Y789C, Y789D, Y789I, Y789V, I790A, I790C, I790F, I790R, I790V, E791A, E791D, E791F, E791M, E791S, E791T, E791V, E791W, V792C, V792G, V792L, V792S, V792Y, D793C, D793F, D793H, D793K, D793N, D793Y, V794C, V794D, V794L, V794Q, V794T, V794W, T795P, T795Y, D796M, D796Q, D796S, D796T, A797H, A797K, N798G, N798I, N798P, N798Q, G799D, G799K, G799L, G799M, G799Q, G799Y, H800A, H800F, H800G, H800L, H800S, H800V, I801C, I801E, I801W, V802E, V802I, V802P, V802S, V802Y, P803A, P803F, P803G, P803K, P803S, P803Y, D804E, D804G, D804K, D804N, D804S, A805C, A805F, A805G, A805I, A805N, A805P, A806F, A806I, A806Q, N807F, N807Q, N807V, N807W, R808C, R808F, R808G, R808I, R808N, R808P, R808Q, V809A, V809C, V809L, V809M, V809P, T810L, T810P, T810Q, T810R, T810Y, F811L, F811Y, D812E, D812F, D812I, D812Q, V813F, V813H, V813T, V813W, V813Y, K814G, K814H, K814I, K814L, K814P, G815A, G815F, G815M, G815P, G815V, A816C, A816D, A816F, A816I, A816N, A816V, A816W, G817C, G817H, G817I, G817N, G817S, K818D, K818F, K818L, K818Q, K818R, K818S, K818V, K818W, K818Y, L819F, L819W, V820C, V820F, V820I, V820K, V820R, V820W, G821A, G821C, G821E, G821F, G821I, G821K, G821M, G821N, G821V, G821Y, V822A, V822D, V822E, V822T, D823E, N824A, N824C, N824G, N824Q, G825A, S826A, S826F, S826G, S826I, S826L, S826R, S826W, S827C, S827Q, P828C, P828G, P828I, P828L, P828Y, D829C, D829I, D829S, D829T, D829V, H830E, H830G, H830M, H830P, H830Q, H830R, H830V, D831A, D831F, D831G, D831I, D831M, D831P, D831R, D831V, S832E, S832F, S832G, S832L, S832M, S832P, S832R, S832V, S832W, Y833C, Y833D, Y833E, Y833I, Y833K, Y833N, Y833P, Y833V, Q834F, Q834G, Q834M, A835D, A835E, A835F, A835H, A835K, A835W, D836C, D836E, D836H, D836Q, D836R, D836S, D836T, D836V, D836W, D836Y, N837D, N837F, N837G, N837H, N837L, N837P, N837T, R838D, R838F, R838G, R838K, R838M, R838N, R838S, R838W, K839A, K839C, K839D, K839E, K839G, K839L, K839N, K839P, A840G, A840I, A840P, A840V, F841C, F841D, F841I, F841K, F841W, S842A, S842L, S842M, G843A, G843C, K844A, K844G, K844L, K844W, K844Y, V845N, V845W, L846G, L846I, L846M, L846S, A847L, A847T, I848M, V849A, V849L, V849S, V849T, Q850C, Q850G, Q850I, Q850L, Q850T, Q850V, Q850Y, S851C, S851D, S851E, S851L, S851T, T852D, T852G, T852L, K853N, K853P, K853Q, K853V, E854C, E854I, E854M, E854R, A855K, A855V, A855Y, E857P, E857V, I858D, I858E, I858F, I858G, I858K, I858M, I858P, I858Q, I858Y, T859V, V860T, V860Y, T861F, T861I, T861Q, T861V, T861W, A862C, A862P, A862V, K863F, K863I, K863L, K863N, K863W, A864E, A864H, A864K, A864L, D865A, D865G, G866H, G866R, L867W, S870E, S870M, S870R, S870T, T871P, V872C, V872G, K873G, K873Y, I874G, A875R, T877A, V879P, V879S, P880S, G881W, T882A, T882M, T882R, S883L, T884A, E885V, K886E, K886L, K886V, K886W, T887A, T887D, T887F, T887G, T887N, T887R, T887V, V888A, V888D, V888G, R889G, Y892D, Y892P, Y892R, Y893E, Y893G, S894D, S894G, R895M, N896M, Y897V, Y898T, V899G, K900E, K900G, T901G, T901Q, T901R, T901V, T901Y, G902A, G902D, G902F, G902L, G902P, G902Q, G902R, G902S, G902W, N903D, N903I, K904D, K904E, K904M, K904N, K904R, K904S, K904V, K904W, P905A, P905C, P905R, P905V, P905W, P905Y, I906A, I906D, I906S, I906T, I906W, I906Y, L907F, L907S, L907Y, P908C, P908D, P908G, P908I, P908L, P908M, P908T, S909E, S909F, S909G, S909W, S909Y, D910C, D910I, D910S, D910W, V911A, V911S, E912A, E912K, E912L, E912T, E912V, V913G, V913Q, V913R, V913W, R914A, R914E, R914F, R914I, R914K, R914V, R914W, R914Y, Y915A, Y915C, Y915G, Y915I, Y915M, Y915Q, Y915V, S916G, D917C, D917E, D917F, D917L, D917M, D917Q, D917S, G918E, G918H, G918T, G918V, G918W, T919D, T919K, T919Q, T919W, T919Y, S920C, S920E, S920M, S920P, S920R, S920V, S920W, D921C, D921P, D921Q, D921V, R922A, R922G, R922M, R922V, R922W, Q923A, Q923C, Q923E, Q923L, Q923M, Q923V, Q923W, N924A, N924L, N924P, N924Q, N924S, N924W, V925A, V925C, V925E, V925G, V925K, V925N, V925S, V925W, T926G, T926P, T926R, T926S, T926V, T926W, W927C, W927G, W927P, D928A, D928E, D928H, D928L, D928Q, A929C, A929P, A929V, V930A, V930E, V930I, V930K, V930M, V930T, S931G, S931P, S931R, D932F, D932R, D932S, D932T, D932V, D933I, D933R, D933S, Q934S, Q934V, I935A, I935C, I935D, I935E, I935L, I935P, I935V, I935W, A936I, A936L, A936Q, A936R, A936Y, K937G, K937I, K937M, K937P, K937Q, K937R, K937V, A938C, A938H, A938N, A938T, A938V, A938W, G939D, G939K, S940C, S940E, S940M, S940R, S940T, S940V, S940W, F941C, F941M, F941W, S942A, S942E, S942K, S942L, S942P, S942T, S942V, V943A, V943G, V943H, V943Q, V943R, A944D, A944G, A944H, A944P, A944R, A944V, G945E, G945P, G945T, T946A, T946E, T946G, T946L, T946P, T946V, T946W, V947G, V947H, V947L, V947M, V947P, V947R, V947T, A948C, A948I, A948R, A948W, G949A, G949F, G949V, Q950D, Q950G, Q950K, Q950M, Q950W, K951D, K951G, K951P, K951Q, K951S, K951W, K951Y, I952H, I952Q, S953F, S953M, S953N, S953R, S953W, V954D, V954L, V954Q, V954S, V954T, R955A, R955C, R955E, R955K, R955Q, R955W, V956A, V956D, V956G, V956H, V956I, V956M, V956Q, V956W, T957D, T957S, T957W, M958D, M958I, M958K, I959A, I959L, I959S, I959V, I959Y, D960G, D960H, D960L, D960P, D960S, D960W, E961D, E961F, E961K, E961P, E961S, E961T, I962A, I962C, I962D, I962G, I962K, I962N, I962Q, I962T, G963C, G963E, G963L, G963P, A964C, A964E, A964H, L965C, L965E, L965G, L965K, L965M, L965P, L965Q, L965S, L965V, L965Y, L966A, L966G, L966H, L966K, L966N, L966P, L966Q, L966S, L966T, L966V, N967C, N967D, N967I, N967L, N967M, N967P, N967S, N967T, N967V, Y968G, Y968L, Y968Q, Y968V, S969C, S969D, S969G, S969H, S969I, S969L, S969M, S969P, S969Q, S969Y, A970I, A970L, S971E, S971F, S971G, S971H, S971V, S971W, P973C, P973D, P973K, P973N, P973Q, P973R, P973V, P973W, P973Y, V974C, V974E, V974G, V974N, V974T, V974Y, G975D, G975F, G975K, G975L, G975Q, G975V, T976D, T976F, T976G, T976K, T976L, T976P, T976S, T976Y, P977A, P977C, P977K, P977R, P977T, P977Y, A978F, A978G, A978M, A978N, A978P, A978R, A978S, A978Y, V979G, V979N, V979R, V979Y, L980A, L980F, L980H, L980I, L980K, L980N, L980Q, L980T, L980Y, P981L, P981M, G982A, G982H, G982M, G982P, G982Q, G982W, R984P, R984S, P985E, P985F, P985H, P985K, P985L, P985W, A986C, A986E, A986F, A986I, A986K, A986L, A986M, A986N, A986S, A986W, V987A, V987C, V987F, V987I, V987K, V987L, V987Q, V987T, L988A, L988C, L988E, L988G, L988H, L988M, L988Q, L988R, L988S, L988V, L988Y, P989A, P989C, P989D, P989G, P989H, P989I, P989M, P989N, P989Q, P989W, D990F, D990P, D990S, D990W, G991C, G991F, G991H, G991K, G991P, G991Y, T992E, T992H, T992M, T992N, T992Y, V993D, V993G, V993N, V993S, T994I, T994S, T994V, S995E, S995L, S995R, S995V, A996Q, A996R, A996V, N997A, N997C, N997E, N997K, N997L, N997S, N997V, N997W, N997Y, F998M, F998W, A999F, A999G, A999L, A999M, A999R, A999S, V1000C, V1000L, V1000M, V1000N, V1000P, V1000W, D1001G, D1001K, D1001L, D1001M, D1001Q, D1001S, D1001T, D1001V, D1001Y, W1002A, W1002D, W1002E, W1002H, W1002N, W1002P, W1002Q, W1002S, T1003F, T1003G, T1003L, T1003N, T1003P, T1003R, T1003S, T1003W, T1003Y, K1004D, K1004E, K1004F, K1004G, K1004H, K1004M, K1004P, K1004R, K1004S, K1004V, P1005I, P1005N, P1005Q, P1005V, P1005Y, A1006C, A1006I, A1006N, A1006P, A1006S, A1006V, A1006W, A1006Y, D1007C, D1007L, D1007P, D1007V, T1008G, V1009G, V1009S, Y1010A, Y1010P, Y1010R, Y1010T, N1011A, N1011S, N1011T, N1011W, T1012E, T1012H, T1012I, T1012Q, T1012Y, A1013D, A1013K, A1013Q, A1013T, A1013V, G1014E, G1014I, G1014L, G1014M, G1014V, G1014W, G1014Y, T1015A, T1015F, T1015G, T1015V, V1016C, V1016D, V1016P, K1017E, K1017G, V1018I, V1018K, V1018L, V1018M, V1018R, V1018S, V1018W, T1021C, T1021E, T1021F, T1021G, T1021K, T1021L, T1021S, T1021V, A1022H, A1022L, A1022S, A1022Y, T1023D, T1023M, T1023Q, T1023R, V1024E, V1024G, V1024H, V1024K, V1024N, V1024R, V1024S, V1024W, G1026E, G1026H, G1026L, G1026R, G1026S, G1026V, G1026Y, K1027C, K1027N, K1027Q, K1027R, K1027V, E1028G, E1028S, E1028T, F1029I, F1029K, F1029L, F1029P, F1029V, F1029W, F1029Y, K1030D, K1030F, K1030H, K1030L, K1030M, K1030W, V1031H, V1031K, V1031Y, A1033G, A1033S, A1033V, T1034G, T1034H, T1034N, T1034W, I1035D, I1035G, I1035Q, R1036G, R1036L, R1036T, R1036Y, V1037C, V1037F, V1037P, V1037Q, Q1038A, Q1038D, Q1038K, R1039S, R1039V, S1040A, S1040M, S1040N, S1040R, S1040W, Q1041P, V1042N, T1043F, T1043G, T1043N, T1043R, I1044A, I1044L, G1045S, S1046I, S1046M, S1047D, V1048C, V1048F, V1048G, V1048I, V1048M, V1048Q, G1050L, G1050S, G1050V, N1051A, N1051E, N1051K, A1052C, A1052K, A1052M, A1052P, A1052R, L1053A, L1053W, R1054C, R1054L, R1054N, L1055R, L1055T, Q1057A, Q1057E, Q1057P, Q1057R, N1058R, N1058S, N1058V, N1058W, I1059W, P1060G, P1060N, P1060Q, P1060S, P1060T, A1061E, A1061G, A1061K, A1061W, D1062A, D1062F, D1062G, D1062I, D1062L, D1062M, D1062P, D1062S, K1063D, K1063M, Q1064C, Q1064M, Q1064R, Q1064T, Q1064V, S1065A, S1065C, S1065E, S1065G, S1065T, S1065W, D1066A, D1066G, D1066M, D1066V, D1066W, T1067G, T1067M, L1068C, L1068E, L1068P, L1068Q, L1068Y, D1069G, D1069K, D1069R, D1069W, A1070P, A1070T, I1071M, I1071R, I1071W, K1072E, K1072G, K1072P, K1072Q, K1072S, D1073F, D1073L, D1073M, D1073P, D1073W, G1074I, G1074L, G1074R, S1075C, S1075G, S1075I, S1075L, S1075V, T1076C, T1076E, T1076H, T1076Q, T1076S, T1077K, T1077L, T1077R, V1078D, V1078E, V1078L, V1078W, D1079G, D1079L, N1081D, N1081E, N1081G, T1082A, T1082C, T1082E, T1082F, T1082G, T1082K, T1082N, T1082S, G1083E, G1083F, G1083L, G1083P, G1083S, G1084C, G1084M, G1084V, G1084W, G1084Y, G1085A, G1085P, G1085R, G1085S, A1086H, A1086K, A1086Q, A1086R, A1086T, N1087A, N1087E, N1087I, N1087R, N1087V, N1087W, P1088D, P1088E, P1088G, P1088R, P1088W, S1089C, S1089E, S1089G, S1089K, S1089Q, S1089R, S1089V, A1090F, A1090G, A1090I, A1090K, W1091A, W1091E, W1091G, W1091H, W1091T, W1091V, W1091Y, T1092A, T1092E, T1092G, T1092K, T1092Q, T1092S, T1092V, N1093A, N1093G, N1093L, N1093P, N1093Q, N1093T, N1093V, W1094D, W1094E, W1094P, W1094R, W1094T, A1095P, A1095R, A1095T, A1095W, Y1096A, Y1096D, Y1096H, Y1096L, Y1096R, S1097D, S1097E, S1097K, S1097L, S1097T, S1097W, K1098D, K1098F, K1098G, K1098Q, K1098S, A1099C, A1099D, A1099F, A1099S, A1099V, A1099W, G1100D, G1100E, G1100H, G1100M, G1100N, G1100T, H1101K, H1101L, H1101Q, H1101R, H1101V, N1102E, N1102F, N1102H, N1102K, N1102L, N1102Q, N1102R, N1102T, T1103A, T1103E, T1103H, T1103S, T1103W, A1104I, A1104K, A1104R, E1105L, E1105S, I1106T, I1106V, T1107C, T1107M, T1107R, T1107S, F1108D, F1108K, F1108L, F1108T, F1108W, E1109A, E1109D, E1109L, E1109W, A1111G, A1111S, E1113D, E1113G, E1113P, E1113V, Q1114E, Q1114I, Q1114L, Q1114R, Q1114S, Q1114T, Q1114V, Q1115A, Q1115K, Q1115L, Q1115P, Q1115T, Q1115W, L1116D, L1116G, L1116H, L1116K, L1116V, L1116W, G1117E, G1117I, G1117M, G1117R, G1117S, G1117T, G1117V, G1117W, Q1118A, Q1118M, Q1118S, Q1118T, Q1118W, I1119D, I1119E, I1119G, I1119N, I1119S, V1120N, V1120S, V1120T, M1121G, M1121K, M1121N, M1121P, M1121S, M1121V, M1121Y, Y1122A, Y1122C, Y1122I, Y1122K, Y1122R, Y1122V, Y1122W, F1123E, F1123H, F1123R, F1123T, F1124E, F1124R, F1124V, F1124W, R1125D, R1125E, R1125F, R1125K, R1125T, R1125V, R1125W, D1126H, D1126K, D1126L, D1126R, S1127F, S1127I, S1127K, S1127M, S1127Q, S1127T, S1127W, N1128A, N1128C, N1128R, N1128S, N1128T, N1128W, A1129E, A1129L, A1129N, A1129Q, A1129R, A1129V, V1130A, V1130G, V1130P, V1130R, V1130S, R1131A, R1131N, R1131Q, R1131S, R1131W, F1132E, F1132K, F1132M, F1132P, F1132Q, F1132T, P1133D, P1133G, P1133L, P1133Q, P1133R, P1133V, D1134E, D1134G, D1134L, A1135E, A1135K, A1135L, A1135M, A1135S, A1135W, A1135Y, G1136A, G1136E, G1136P, G1136Q, G1136T, K1137A, K1137C, K1137G, K1137L, K1137P, K1137Q, K1137R, K1137S, K1137T, K1137V, T1138R, T1138Y, K1139A, K1139L, K1139R, K1139T, I1140A, I1140C, I1140G, I1140L, I1140M, I1140P, I1140R, I1140T, Q1141A, Q1141C, Q1141G, Q1141K, Q1141N, Q1141P, Q1141T, Q1141W, I1142E, I1142R, I1142S, I1142W, I1142Y, S1143G, A1144C, A1144D, A1144E, A1144N, A1144P, A1144S, A1144T, A1144V, A1144W, D1145E, D1145R, D1145T, G1146A, G1146C, G1146D, G1146K, G1146L, G1146R, G1146V, K1147A, K1147G, K1147T, K1147V, N1148H, N1148I, N1148K, N1148P, N1148Q, N1148R, N1148S, N1148T, N1148W, W1149C, W1149G, W1149I, W1149K, W1149N, W1149Q, W1149S, W1149T, W1149V, W1149Y, T1150G, T1150K, T1150P, D1151C, D1151G, D1151R, D1151T, D1151W, L1152A, L1152C, L1152E, L1152Q, L1152W, A1153E, A1153G, A1153K, A1153L, A1154C, A1154D, A1154E, A1154G, A1154R, A1154S, T1155E, T1155L, T1155Q, T1155R, T1157V, T1157W, I1158R, I1158S, I1158W, A1159C, A1159E, A1159I, A1159P, A1159R, A1159V, A1160K, A1160L, A1160Q, A1160S, Q1161A, Q1161P, Q1161S, E1162A, E1162C, E1162D, E1162F, E1162I, E1162N, E1162Q, E1162T, E1162W, E1162Y, E1165H, E1165L, E1165M, E1165R, E1165S, E1165W, R1166D, R1166K, R1166Q, V1167A, V1167C, V1167L, V1167P, V1167R, K1168L, K1168Q, K1168R, K1168W, P1169M, P1169R, P1169S, Y1170E, Y1170K, Y1170M, Y1170Q, Y1170R, Y1170V, T1171A, T1171G, T1171M, T1171Q, T1171R, T1171S, Y1172D, Y1172E, Y1172H, Y1172I, Y1172K, Y1172L, Y1172S, Y1172V, D1173A, D1173E, D1173F, D1173G, D1173K, D1173L, D1173P, D1173R, D1173T, D1173W, F1174D, F1174P, F1174Q, F1174R, F1174S, F1174T, F1174V, F1174W, A1175G, A1175I, A1175N, A1175Q, A1175S, A1175V, A1175Y, V1177N, V1177P, V1177S, V1177T, G1178M, G1178Q, G1178S, G1178T, A1179L, A1179P, A1179Q, A1179W, T1180A, T1180G, T1180I, T1180L, T1180M, T1180Q, T1180S, T1180Y, F1181E, F1181L, F1181V, V1182M, K1183A, K1183E, K1183T, K1183V, V1184C, V1184E, V1184K, V1184L, V1184P, V1184Q, V1184Y, T1185Q, T1185V, V1186S, N1188V, N1188W, A1189C, A1189K, A1189T, A1189V, D1190R, D1190S, D1190T, D1190Y, T1191E, T1191L, T1192H, T1192P, T1193G, P1194A, P1194E, P1194G, P1194W, S1195G, V1197A, V1198E, C1199D, C1199T, A1200G, A1200W, L1202A, L1202C, T1203E, T1203K, E1204G, E1204S, I1205V, K1208R, K1208V, K1208W, T1209A, T1209C, T1209E, T1209K, T1209N, T1209Q, T1209R, T1209W, A1210D, A1210E, A1210G, A1210K, A1210L, A1210Q, A1210R, A1210T, A1210W, T1211C, T1211D, T1211E, T1211G, T1211H, T1211K, T1211P, T1211Q, T1211R, T1211S, T1211V, K1213A, K1213D, K1213S, K1213T, K1213W, F1214A, F1214E, F1214K, F1214L, F1214P, F1214R, F1214S, F1214V, V1215D, V1215E, V1215K, V1215L, V1215Q, V1215S, V1215W, T1216L, T1216P, T1216Q, T1216R, N1217A, N1217D, N1217E, N1217F, N1217P, N1217R, N1217S, N1217T, T1218A, T1218C, T1218E, T1218G, T1218Q, T1218S, T1218V, T1218W, S1219A, S1219E, S1219F, S1219I, S1219K, S1219R, S1219V, A1220C, A1220G, A1220L, A1220P, A1220R, A1220V, A1221D, A1221G, A1221K, A1221L, A1221R, A1221V, A1221W, L1222A, L1222C, L1222E, L1222F, L1222Q, L1222R, L1222V, L1222W, S1223C, S1223F, S1223G, S1223K, S1223L, S1223V, S1224A, S1224D, S1224G, S1224L, S1224M, S1224P, S1224R, S1224W, L1225C, L1225D, L1225E, L1225F, L1225G, L1225K, L1225P, L1225T, L1225V, L1225W, T1226A, T1226G, T1226M, T1226P, T1226R, T1226S, T1226V, T1226Y, V1227A, V1227C, V1227D, V1227E, V1227G, V1227L, V1227P, V1227Q, V1227S, N1228A, N1228D, N1228F, N1228K, N1228L, N1228T, G1229A, G1229C, G1229E, G1229Q, G1229S, G1229V, T1230F, T1230H, T1230I, T1230K, T1230L, T1230P, T1230R, T1230S, T1230W, K1231F, K1231G, K1231L, K1231M, K1231P, K1231S, K1231W, V1232E, V1232K, V1232Q, V1232R, V1232S, V1232T, V1232W, S1233P, S1233W, D1234G, D1234K, D1234R, D1234V, S1235D, S1235E, S1235G, S1235L, S1235P, S1235R, S1235W, S1235Y, V1236A, V1236C, V1236G, V1236I, V1236P, V1236Q, V1236R, L1237D, L1237E, L1237R, L1237V, L1237W, A1238D, A1238E, A1238K, A1238L, A1238N, A1238P, A1238R, A1238S, A1238T, A1239D, A1239P, A1239R, G1240D, G1240L, G1240N, G1240Q, G1240S, G1240T, G1240W, S1241D, S1241G, S1241I, S1241L, S1241M, S1241P, Y1242C, Y1242E, Y1242K, Y1242R, Y1242S, Y1242W, N1243C, N1243L, N1243M, N1243P, N1243Q, N1243S, N1243T, N1243V, N1243W, T1244A, T1244D, T1244E, T1244G, T1244L, T1244Q, T1244S, T1244V, T1244W, A1246F, A1246M, A1246N, A1246P, A1246Q, A1246R, A1246S, A1246T, I1247A, I1247G, I1247M, I1247Q, I1247R, I1247S, I1247T, I1247V, I1247W, I1248A, I1248G, I1248K, I1248L, I1248R, I1248S, I1248Y, A1249E, A1249G, A1249H, A1249I, A1249R, A1249T, A1249V, D1250I, D1250K, D1250S, D1250T, D1250W, D1250Y, V1251I, V1251T, V1251W, K1252D, K1252G, K1252V, K1252W, A1253P, A1253V, E1254F, E1254G, E1254H, E1254L, E1254R, E1254V, G1255H, G1255M, G1255S, G1255V, E1256G, E1256M, E1256N, E1256R, E1256V, E1256W, G1257F, G1257K, G1257L, G1257Q, G1257R, G1257W, N1258C, N1258G, N1258H, N1258K, N1258S, A1259K, A1259L, A1259W, V1261I, V1261L, V1261P, V1261Q, V1261R, V1261T, T1262A, T1262F, T1262M, T1262Q, T1262R, V1263E, V1263G, V1263Q, V1263R, V1263T, V1263W, L1264A, L1264E, L1264H, L1264R, L1264S, L1264Y, P1265C, P1265K, P1265L, P1265R, P1265S, P1265V, P1265W, A1266F, A1266L, A1266P, A1266S, A1266V, H1267A, H1267E, H1267F, N1269A, N1269E, N1269K, N1269R, N1269S, N1269T, N1269W, V1270D, V1270E, V1270G, V1270I, V1270L, V1270T, V1270W, I1271A, I1271H, I1271Q, R1272E, R1272F, R1272M, R1272P, R1272V, V1273R, I1274F, I1274M, I1274R, T1275A, T1275L, T1275W, E1276R, E1276W, S1277L, S1277T, S1277W, E1278Q, E1278R, D1279G, D1279I, D1279R, D1279T, D1279V, D1279W, H1280C, H1280E, H1280G, H1280V, H1280W, V1281F, V1281I, V1281S, V1281W, T1282D, T1282L, T1282V, R1283A, R1283D, R1283E, R1283P, R1283W, K1284G, T1285A, T1285E, T1285F, T1285G, T1285M, T1285R, T1285Y, F1286A, F1286E, F1286P, F1286R, F1286S, F1286T, T1287C, T1287K, T1287L, T1287M, T1287Q, T1287R, T1287S, T1287W, I1288A, I1288D, I1288F, I1288G, I1288K, N1289A, N1289Q, N1289T, L1290A, L1290G, L1290R, L1290V, L1290W, G1291K, G1291P, G1291V, G1291W, G1291Y, T1292G, T1292L, T1292Y, E1293G, E1293K, E1293L, E1293S, E1293V, Q1294E, Q1294L, Q1294P, Q1294W, E1295K, F1296A, F1296G, F1296I, P1297F, A1298Y, D1301I, E1302G, E1302R, E1302S, R1303S, D1304A or D1304V.

Preferably, the lactase variant has a decreased or an increased galactose inhibition, preferably measured as a galactose inhibition improvement factor (GI-IF) of at least 1.1 or at most 0.9, compared to the lactase of SEQ ID NO: 1.

The invention is also directed to an isolated polynucleotide encoding the variant, a nucleic acid construct, an expression vector, a recombinant host cell, and a method of producing a lactase variant of the invention.

The invention is also directed to a method for producing a dairy product comprising:
a) providing a milk-based substrate comprising lactose; and
b) treating said substrate with a variant of the invention.

The invention is also directed to use of such variant for production of a dairy product.

The invention is also directed to a liquid composition comprising such variant and 20-70% glycerol.

The invention is also directed to a method for obtaining a lactase variant, comprising: a) introducing into a parent lactase having an amino acid sequence which is at least 80%, 85%, 90%, or 95%, preferably at least 97%, 98%, or 99%, more preferably at least 99.5%, 99.7%, 99.8%, 99.9%, or 100%, identical to SEQ ID NO: 1 an alteration, preferably a substitution, at one or more positions corresponding to positions 1, 2, 3, 4, 5, 6, 7, 9, 10, 11, 12, 13, 14, 15, 16, 17, 19, 20, 21, 22, 23, 24, 25, 26, 27, 28, 29, 30, 31, 32, 33, 34, 35, 36, 37, 38, 39, 40, 41, 42, 43, 44, 45, 47, 48, 49, 51, 53, 54, 55, 57, 58, 59, 60, 61, 62, 63, 64, 66, 67, 68, 70, 71, 73, 74, 75, 76, 77, 78, 79, 80, 81, 83, 85, 86, 87, 88, 89, 90, 91, 92, 93, 94, 95, 96, 97, 98, 100, 101, 102, 104, 105, 106, 107, 108, 109, 110, 111, 112, 113, 114, 115, 116, 117, 118, 119, 120, 121, 122, 123, 124, 125, 126, 128, 129, 130, 131, 132, 133, 135, 136, 137, 138, 139, 140, 141, 142, 146, 148, 149, 150, 151, 153, 154, 155, 157, 158, 159, 160, 161, 162, 164, 165, 166, 167, 168, 169, 170, 171, 172, 173, 174, 175, 176, 177, 178, 179, 180, 181, 182, 183, 184, 185, 186, 187, 188, 189, 190, 191, 193, 194, 195, 196, 197, 198, 199, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 254, 255, 256, 257, 258, 259, 260, 261, 262, 263, 264, 265, 266, 267, 268, 269, 270, 271, 272, 273, 274, 275, 277, 278, 279, 280, 281, 282, 283, 284, 285, 286, 287, 288, 291, 292, 293, 294, 295, 296, 297, 298, 299, 301, 302, 303, 304, 305, 306, 307, 308, 309, 310, 311, 312, 314, 315, 316, 317, 318, 320, 321, 322, 324, 325, 326, 328, 329, 331, 335, 337, 338, 340, 341, 342, 343, 344, 345, 346, 347, 348, 349, 350, 351, 352, 353, 354, 355, 357, 359, 360, 362, 363, 364, 365, 366, 367, 368, 369, 370, 371, 372, 373, 374, 375, 376, 377, 378, 379, 380, 381, 383, 386, 387, 388, 389, 390, 391, 392, 393, 394, 395, 396, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 485, 487, 488, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 534, 535, 537, 538, 539, 540, 542, 543, 545, 546, 547, 548, 549, 550, 551, 553, 554, 555, 556, 557, 558, 559, 560, 561, 563, 564, 565, 567, 570, 571, 572, 573, 574, 575, 576, 577, 578, 579, 580, 581, 582, 583, 584, 585, 586, 587, 588, 589, 590, 591, 592, 593, 594, 595, 597, 598, 600, 604, 605, 606, 607, 608, 609, 610, 611, 612, 613, 614, 615, 616, 619, 620, 621, 624, 625, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 659, 661, 662, 667, 669, 670, 672, 673, 674, 675, 676, 677, 678, 679, 680, 681, 682, 683, 684, 685, 686, 687, 688, 689, 690, 691, 692, 693, 694, 695, 696, 697, 698, 700, 701, 703, 704, 705, 706, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 765, 766, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 857, 858, 859, 860, 861, 862, 863, 864, 865, 866, 867, 870, 871, 872, 873, 874, 875, 877, 879, 880, 881, 882, 883, 884, 885, 886, 887, 888, 889, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 973, 974, 975, 976, 977, 978, 979, 980, 981, 982, 984, 985, 986, 987, 988, 989, 990, 991, 992, 993, 994, 995, 996, 997, 998, 999, 1000, 1001, 1002, 1003, 1004, 1005, 1006, 1007, 1008, 1009, 1010, 1011, 1012, 1013, 1014, 1015, 1016, 1017, 1018, 1021, 1022, 1023, 1024, 1026, 1027, 1028, 1029, 1030, 1031, 1033, 1034, 1035, 1036, 1037, 1038, 1039, 1040, 1041, 1042, 1043, 1044, 1045, 1046, 1047, 1048, 1050, 1051, 1052, 1053, 1054, 1055, 1057, 1058, 1059, 1060, 1061, 1062, 1063, 1064, 1065, 1066, 1067, 1068, 1069, 1070, 1071, 1072, 1073, 1074, 1075, 1076, 1077, 1078, 1079, 1081, 1082, 1083, 1084, 1085, 1086, 1087, 1088, 1089, 1090, 1091, 1092, 1093, 1094, 1095, 1096, 1097, 1098, 1099, 1100, 1101, 1102, 1103, 1104, 1105, 1106, 1107, 1108, 1109, 1111, 1113, 1114, 1115, 1116, 1117, 1118, 1119, 1120, 1121, 1122, 1123, 1124, 1125, 1126, 1127, 1128, 1129, 1130, 1131, 1132, 1133, 1134, 1135, 1136, 1137, 1138, 1139, 1140, 1141, 1142, 1143, 1144, 1145, 1146, 1147, 1148, 1149, 1150, 1151, 1152, 1153, 1154, 1155, 1157, 1158, 1159, 1160, 1161, 1162, 1165, 1166, 1167, 1168, 1169, 1170, 1171, 1172, 1173, 1174, 1175, 1177, 1178, 1179, 1180, 1181, 1182, 1183, 1184, 1185, 1186, 1188, 1189, 1190, 1191, 1192, 1193, 1194, 1195, 1197, 1198, 1199, 1200, 1202, 1203, 1204, 1205, 1208, 1209, 1210, 1211, 1213, 1214, 1215, 1216, 1217, 1218, 1219, 1220, 1221, 1222, 1223, 1224, 1225, 1226, 1227, 1228, 1229, 1230, 1231, 1232, 1233, 1234, 1235, 1236, 1237, 1238, 1239, 1240, 1241, 1242, 1243, 1244, 1246, 1247, 1248, 1249, 1250, 1251, 1252, 1253, 1254, 1255, 1256, 1257, 1258, 1259, 1261, 1262, 1263, 1264, 1265, 1266, 1267, 1269, 1270, 1271, 1272, 1273, 1274, 1275, 1276, 1277, 1278, 1279, 1280, 1281, 1282, 1283, 1284, 1285, 1286, 1287, 1288, 1289, 1290, 1291, 1292, 1293, 1294, 1295, 1296, 1297, 1298, 1301, 1302, 1303 or 1304 of the polypeptide of SEQ ID NO: 1, and
b) recovering the variant.

### DETAILED DESCRIPTION OF THE INVENTION

### Definitions

Sequence identity: The relatedness between two amino acid sequences is described by the parameter “sequence identity”.

For purposes of the present invention, the sequence identity between two amino acid sequences is determined as the output of “longest identity” using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 6.6.0 or later. The parameters used are a gap open penalty of 10, a gap extension penalty of 0.5, and the EBLOSUM62 (EMBOSS version of BLOSUM62) substitution matrix. In order for the Needle program to report the longest identity, the nobrief option must be specified in the command line. The output of Needle labeled “longest identity” is calculated as follows:

(Identical Residues×100)/(Length of Alignment−Total Number of Gaps in Alignment)

Conventions for Designation of Variants: For purposes of the present invention, the polypeptide disclosed in SEQ ID NO: 1 is used to determine the corresponding amino acid position in another enzyme. The amino acid sequence of another enzyme is aligned with the polypeptide disclosed in SEQ ID NO: 1, and based on the alignment, the amino acid position number corresponding to any amino acid residue in the polypeptide disclosed in SEQ ID NO: 1 is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 6.6.0 or later. The parameters used are gap open penalty of 10, gap extension penalty of 0.5, and the EBLOSUM62 (EMBOSS version of BLOSUM62) substitution matrix.

In describing the variants of the present invention, the nomenclature described below is adapted for ease of reference. The accepted IUPAC single letter or three letter amino acid abbreviation is employed.

Substitutions. For an amino acid substitution, the following nomenclature is used: Original amino acid, position, substituted amino acid. Accordingly, the substitution of threonine at position 226 with alanine is designated as “Thr226Ala” or “T226A”. Multiple mutations may be separated by addition marks (“+”), e.g., “Gly205Arg+Ser411Phe” or “G205R+S411F”, representing substitutions at positions 205 and 411 of glycine (G) with arginine (R) and serine(S) with phenylalanine (F), respectively.

Different substitutions. Where different substitutions can be introduced at a position, the different substitutions may be separated by a comma, e.g., “Arg170Tyr, Glu” represents a substitution of arginine at position 170 with tyrosine or glutamic acid. Thus, “Tyr167Gly, Ala+Arg170Gly,Ala” designates the following variants:
“Tyr167Gly+Arg170Gly”, “Tyr167Gly+Arg170Ala”, “Tyr167Ala+Arg170Gly”, and “Tyr167Ala+Arg170Ala”.

Lactase: A “lactase” in the context of the present invention is any glycoside hydrolase having the ability to hydrolyse the disaccharide lactose into constituent galactose and glucose monomers. The group of lactases comprises but is not limited to enzymes assigned to subclass EC 3.2.1.108. Enzymes assigned to other subclasses, such as, e.g., EC 3.2.1.23, may also be lactases in the context of the present invention. A lactase in the context of the invention may have other activities than the lactose hydrolysing activity, such as for example a transgalactosylating activity. In the context of the invention, the lactose hydrolysing activity of the lactase may be referred to as its lactase activity or its beta-galactosidase activity.

Lactase activity may be determined using, e.g., a LAU-B assay. In the context of the present invention, the activity in LAU-B/g of a specific lactase may be determined by direct measurement of o-nitrophenyl (ONP) released from o-nitrophenyl 3-D-galactopyranoside (ONPG) in a buffer containing 1.46 mg/ml substrate in 0.05 M MES, 1 mM MgSO4 7H2O, 450 mg/L Brij 35 at pH6.5 and 30° C. After 600 seconds incubation, the reaction is stopped by adding 0.2 M Na2CO3 and the released ONP is measured at 405 nm after 126 seconds incubation. The skilled person will know how to execute this assay and determine such activity. Here, the activity is obtained by comparing to a standard curve run with a lactase of known activity, and the activity of the unknown sample calculated from this. The lactase of known activity may, e.g., be Saphera® obtained from Novozymes A/S, Denmark.

Isolated: The term “isolated” means a polypeptide, nucleic acid, cell, or other specified material or component that is separated from at least one other material or component with which it is naturally associated as found in nature, including but not limited to, for example, other proteins, nucleic acids, cells, etc. An isolated polypeptide includes, but is not limited to, a culture broth containing the secreted polypeptide.

Variant: The term “variant” or “lactase variant” means a polypeptide having lactase activity comprising a substitution, an insertion, and/or a deletion, at one or more (e.g., several) positions compared to a reference sequence. Variants of the present invention preferably comprise a substitution at one or more positions. A substitution means replacement of the amino acid occupying a position with a different amino acid; a deletion means removal of the amino acid occupying a position; and an insertion means adding one or more amino acid(s) adjacent to and immediately following the amino acid occupying a position. The variants of the present invention may have at least 20%, e.g., at least 40%, at least 50%, at least 60%, at least 70%, at least 80%, at least 90%, at least 95%, or at least 100% of the lactase activity of the polypeptide of SEQ ID NO: 1. A lactase variant is thus a variant compared to a reference sequence.

Parent: The term “parent” lactase or lactase “parent” is to be understood as a lactase into which at least one alteration is made in the amino acid sequence to produce a lactase variant having an amino acid sequence which is less than 100% identical to the amino acid sequence of the parent lactase. In a particular aspect the lactase parent is a lactase having an amino acid sequence which is at least 80%, at least 85%, at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9% or 100% identical to SEQ

Milk: The term “milk” means the lacteal secretion obtained by milking any mammal, such as cows, sheep, goats, buffaloes or camels. Preferably milk is cow's milk.

Milk-based substrate: The term “milk-based substrate”, in the context of the present invention, may be any raw and/or processed milk material. Useful milk-based substrates include, but are not limited to, solutions/suspensions of any milk or milk like products comprising lactose, such as whole or low fat milk, skim milk, buttermilk, reconstituted milk powder, condensed milk, solutions of dried milk, UHT milk, whey, whey permeate, acid whey, or cream. Preferably, the milk-based substrate is milk or an aqueous solution of skim milk powder.

Dairy product: A “dairy product” in the context of the present invention may be any food product wherein one of the major constituents is milk-based. Preferable, the major constituent is milk-based. More preferably, the major constituent is a milk-based substrate which has been treated with an enzyme having lactase activity according to the invention. In the context of the present invention “one of the major constituents” means a constituent having a dry matter which constitutes more than 20%, preferably more than 30% or more than 40% of the total dry matter of the dairy product, whereas “the major constituent” means a constituent having a dry matter which constitutes more than 50%, preferably more than 75% or more than 80%, more preferably more than 90% or more than 95%, of the total dry matter of the dairy product.

### Lactase Variants

The present invention provides novel lactases. In a preferred embodiment, the lactases are obtained from Bifidobacterium, in particular Bifidobacterium bifidum. In another preferred embodiment, the lactases may be variants of parent lactases, preferably obtained from Bifidobacterium, in particular Bifidobacterium bifidum.

The lactase variants of the present invention are variants compared to a reference sequence, preferably SEQ ID NO: 1.

The lactase variants of the invention preferably have an amino acid sequence which is at least 80%, 85%, 90%, or 95%, preferably at least 97%, 98%, or 99%, more preferably at least 99.5%, 99.7%, 99.8% or 99.9%, but less than 100%, identical to SEQ ID NO: 1.

In an embodiment, the lactase variants of the invention have an amino acid sequence which is at least 80%, 85%, 90%, or 95%, preferably at least 97%, 98%, or 99%, more preferably at least 99.5%, 99.7%, 99.8% or 99.9%, but less than 100%, identical to any of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14 or SEQ ID NO: 15.

The lactase variants of the invention comprise an alteration, preferably a substitution, of at least one amino acid compared to the lactase of SEQ ID NO: 1. In some aspects, a lactase variant of the invention comprise at least two substitutions compared to the lactase of SEQ ID NO: 1, such as at least 3 substitutions, at least 4 substitutions, at least 5 substitutions, such as at least 6, 7, 8, 9 or 10 substitutions.

The lactase variants of the invention may have 1-50 substitutions, preferably 1-30, 1-20, 1-10 or 1-5, such as 1, 2, 3, 4, 5, 6, 7, 8, 9, 10, 11, 12, 13, 14, 15, 16, 17, 18, 19 or 20 substitutions, compared to the lactase of SEQ ID NO: 1.

The lactase variants of the invention have lactase activity.

The lactase variants of the invention may be isolated.

In an embodiment, the lactase variants of the invention have a length of 850-2000 amino acids, such as 880-1350 amino acids.

In a preferred embodiment, the lactase variants of the invention have a length of 1200-2000 amino acids, preferably 1200-1500 amino acids, more preferably 1250-1400 amino acids or 1250-1350 amino acids, even more preferably 1280-1320 amino acids, such as 1300-1320 amino acids or 1300-1310 amino acids, most preferably 1300-1305, such as 1302-1304 amino acids.

In a more preferred embodiment, the lactase variants of the invention comprise the same number of amino acid residues as the lactase of SEQ ID NO: 1.

In another preferred embodiment, the lactase variants of the invention have a ratio of beta-galactosidase activity to transgalactosylating activity of at least 2, preferably at least 3, such as at least 4, more preferably at least 5, such as at least 10, where said ratio is measured as [Galactose]/([Glucose]−[Galactose]) after incubation of the enzyme in cow's milk at 37° C. for 3 hours.

The lactase variants of the present invention preferably have at least one improved property compared to a reference lactase. The reference lactase may be the parent lactase from which the variant is obtained. In a preferred embodiment, the reference lactase is the lactase of SEQ ID NO: 1.

In a preferred embodiment, the lactase variants of the present invention have a galactose inhibition improvement factor (GI-IF) of at least 1.1 or at most 0.9, compared to the lactase of SEQ ID NO: 1. The GI-IF is preferably calculated as described in Example 2.

I.e., the galactose inhibition improvement factor (GI-IF) of a given variant may be calculated as the RA (ratio of activity) of the variant relative to the RA of the lactase of SEQ ID NO: 1, which is normalized to have a GI-IF=1.0, where the RA is calculated as (Abs405 Galactose sample-Abs405 Galactose Blank)/(Abs405 Reference sample-Abs405 Reference Blank). The variants and the lactase of SEQ ID NO: 1 may be grown in reference solution (MES (100 mM), ONPG (10.66 mM), DMSO (140 mM), pH 6.0) and assay solution (reference solution plus D-(+)-Galactose (80 mM)) for 30 min at 23° C. Stop solution is added (Na2CO3, 10% w/v in H2O), and the absorbance at 405 nm is recorded. “Abs405 Galactose sample” is the absorbance for the lactase grown in assay solution, “Abs405 Galactose Blank” is the absorbance for non-inoculated assay solution, “Abs405 Reference sample” is the absorbance for the lactase grown in reference solution, and “Abs405 Reference Blank” is the absorbance for non-inoculated reference solution.

A GI-IF above 1 means that the variant has a decreased galactose inhibition compared to the reference lactase of SEQ ID NO: 1, whereas a GI-IF below 1 means that the variant has an increased galactose inhibition compared to the reference lactase of SEQ ID NO: 1.

Variants showing decreased galactose inhibition (i.e. GI-IF of at least 1.9, or at least 1.8, or at least 1.7, or at least 1.6, or at least 1.5, or at least 1.4, or at least 1.3, or at least 1.2, or at least 1.1, but more than 1.0) are beneficial in application, since they lead to higher lactase performance. Moreover, variants showing increased galactose inhibition (i.e. GI-IF of at most 0.1, or at most 0.2, or at most 0.3, or at most 0.4, or at most 0.5, or at most 0.6, or at most 0.7, or at most 0.8, or at most 0.9, but less than 1.0) are also beneficial in application, since they allow to fine-tune the galactose inhibition propensity of the lactase.

The lactase variants of the present invention may have at least one other improved property compared to a reference lactase, preferably the lactase of SEQ ID NO: 1.

The lactase variants of the present invention may have at least one other improved property compared to the parent lactase, preferably the lactase of SEQ ID NO: 1.

Such properties may include one or more of the following: Increased specific activity, increased thermostability, decreased thermostability, or increased acidic stability.

An increased specific activity may be advantageous, e.g., because more lactose hydrolysis can be obtained using a smaller amount of enzyme.

An increased thermostability allows for treatment of the milk or dairy product at higher temperature. This may reduce the microbial count and thereby improve the quality of the milk or dairy product.

A decreased thermostability may allow for using a lower temperature for thermal inactivation of the lactase.

An increased acidic stability is especially useful for the production of fermented dairy products, since the enzyme is still active during and after fermentation.

The lactase variants of the present invention comprise an alteration, preferably a substitution, at one or more positions corresponding to positions 1, 2, 3, 4, 5, 6, 7, 9, 10, 11, 12, 13, 14, 15, 16, 17, 19, 20, 21, 22, 23, 24, 25, 26, 27, 28, 29, 30, 31, 32, 33, 34, 35, 36, 37, 38, 39, 40, 41, 42, 43, 44, 45, 47, 48, 49, 51, 53, 54, 55, 57, 58, 59, 60, 61, 62, 63, 64, 66, 67, 68, 70, 71, 73, 74, 75, 76, 77, 78, 79, 80, 81, 83, 85, 86, 87, 88, 89, 90, 91, 92, 93, 94, 95, 96, 97, 98, 100, 101, 102, 104, 105, 106, 107, 108, 109, 110, 111, 112, 113, 114, 115, 116, 117, 118, 119, 120, 121, 122, 123, 124, 125, 126, 128, 129, 130, 131, 132, 133, 135, 136, 137, 138, 139, 140, 141, 142, 146, 148, 149, 150, 151, 153, 154, 155, 157, 158, 159, 160, 161, 162, 164, 165, 166, 167, 168, 169, 170, 171, 172, 173, 174, 175, 176, 177, 178, 179, 180, 181, 182, 183, 184, 185, 186, 187, 188, 189, 190, 191, 193, 194, 195, 196, 197, 198, 199, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 254, 255, 256, 257, 258, 259, 260, 261, 262, 263, 264, 265, 266, 267, 268, 269, 270, 271, 272, 273, 274, 275, 277, 278, 279, 280, 281, 282, 283, 284, 285, 286, 287, 288, 291, 292, 293, 294, 295, 296, 297, 298, 299, 301, 302, 303, 304, 305, 306, 307, 308, 309, 310, 311, 312, 314, 315, 316, 317, 318, 320, 321, 322, 324, 325, 326, 328, 329, 331, 335, 337, 338, 340, 341, 342, 343, 344, 345, 346, 347, 348, 349, 350, 351, 352, 353, 354, 355, 357, 359, 360, 362, 363, 364, 365, 366, 367, 368, 369, 370, 371, 372, 373, 374, 375, 376, 377, 378, 379, 380, 381, 383, 386, 387, 388, 389, 390, 391, 392, 393, 394, 395, 396, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 485, 487, 488, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 534, 535, 537, 538, 539, 540, 542, 543, 545, 546, 547, 548, 549, 550, 551, 553, 554, 555, 556, 557, 558, 559, 560, 561, 563, 564, 565, 567, 570, 571, 572, 573, 574, 575, 576, 577, 578, 579, 580, 581, 582, 583, 584, 585, 586, 587, 588, 589, 590, 591, 592, 593, 594, 595, 597, 598, 600, 604, 605, 606, 607, 608, 609, 610, 611, 612, 613, 614, 615, 616, 619, 620, 621, 624, 625, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 659, 661, 662, 667, 669, 670, 672, 673, 674, 675, 676, 677, 678, 679, 680, 681, 682, 683, 684, 685, 686, 687, 688, 689, 690, 691, 692, 693, 694, 695, 696, 697, 698, 700, 701, 703, 704, 705, 706, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 765, 766, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 857, 858, 859, 860, 861, 862, 863, 864, 865, 866, 867, 870, 871, 872, 873, 874, 875, 877, 879, 880, 881, 882, 883, 884, 885, 886, 887, 888, 889, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 973, 974, 975, 976, 977, 978, 979, 980, 981, 982, 984, 985, 986, 987, 988, 989, 990, 991, 992, 993, 994, 995, 996, 997, 998, 999, 1000, 1001, 1002, 1003, 1004, 1005, 1006, 1007, 1008, 1009, 1010, 1011, 1012, 1013, 1014, 1015, 1016, 1017, 1018, 1021, 1022, 1023, 1024, 1026, 1027, 1028, 1029, 1030, 1031, 1033, 1034, 1035, 1036, 1037, 1038, 1039, 1040, 1041, 1042, 1043, 1044, 1045, 1046, 1047, 1048, 1050, 1051, 1052, 1053, 1054, 1055, 1057, 1058, 1059, 1060, 1061, 1062, 1063, 1064, 1065, 1066, 1067, 1068, 1069, 1070, 1071, 1072, 1073, 1074, 1075, 1076, 1077, 1078, 1079, 1081, 1082, 1083, 1084, 1085, 1086, 1087, 1088, 1089, 1090, 1091, 1092, 1093, 1094, 1095, 1096, 1097, 1098, 1099, 1100, 1101, 1102, 1103, 1104, 1105, 1106, 1107, 1108, 1109, 1111, 1113, 1114, 1115, 1116, 1117, 1118, 1119, 1120, 1121, 1122, 1123, 1124, 1125, 1126, 1127, 1128, 1129, 1130, 1131, 1132, 1133, 1134, 1135, 1136, 1137, 1138, 1139, 1140, 1141, 1142, 1143, 1144, 1145, 1146, 1147, 1148, 1149, 1150, 1151, 1152, 1153, 1154, 1155, 1157, 1158, 1159, 1160, 1161, 1162, 1165, 1166, 1167, 1168, 1169, 1170, 1171, 1172, 1173, 1174, 1175, 1177, 1178, 1179, 1180, 1181, 1182, 1183, 1184, 1185, 1186, 1188, 1189, 1190, 1191, 1192, 1193, 1194, 1195, 1197, 1198, 1199, 1200, 1202, 1203, 1204, 1205, 1208, 1209, 1210, 1211, 1213, 1214, 1215, 1216, 1217, 1218, 1219, 1220, 1221, 1222, 1223, 1224, 1225, 1226, 1227, 1228, 1229, 1230, 1231, 1232, 1233, 1234, 1235, 1236, 1237, 1238, 1239, 1240, 1241, 1242, 1243, 1244, 1246, 1247, 1248, 1249, 1250, 1251, 1252, 1253, 1254, 1255, 1256, 1257, 1258, 1259, 1261, 1262, 1263, 1264, 1265, 1266, 1267, 1269, 1270, 1271, 1272, 1273, 1274, 1275, 1276, 1277, 1278, 1279, 1280, 1281, 1282, 1283, 1284, 1285, 1286, 1287, 1288, 1289, 1290, 1291, 1292, 1293, 1294, 1295, 1296, 1297, 1298, 1301, 1302, 1303 or 1304 of the polypeptide of SEQ ID NO: 1.

In a preferred embodiment, the lactase variants of the present invention comprise an alteration, preferably a substitution, at one or more positions corresponding to positions 2, 3, 4, 5, 6, 7, 9, 10, 11, 12, 13, 14, 15, 16, 17, 21, 22, 23, 24, 25, 26, 27, 28, 29, 31, 32, 33, 34, 35, 36, 37, 38, 39, 40, 41, 42, 43, 44, 45, 47, 48, 49, 51, 54, 55, 57, 58, 59, 60, 61, 62, 63, 64, 66, 67, 68, 70, 71, 73, 74, 75, 76, 77, 78, 79, 80, 81, 83, 85, 86, 87, 88, 89, 90, 91, 92, 93, 94, 95, 96, 97, 98, 100, 101, 102, 104, 105, 106, 107, 108, 109, 110, 111, 112, 113, 114, 115, 116, 117, 118, 119, 120, 121, 122, 123, 124, 125, 126, 128, 129, 130, 131, 132, 133, 135, 136, 137, 138, 139, 140, 141, 142, 146, 148, 150, 151, 153, 154, 155, 157, 158, 159, 160, 161, 162, 164, 165, 166, 167, 168, 169, 170, 171, 172, 173, 174, 175, 176, 177, 178, 179, 180, 181, 182, 183, 184, 185, 186, 187, 188, 189, 190, 191, 193, 194, 195, 196, 197, 198, 199, 201, 202, 203, 204, 206, 207, 208, 209, 210, 211, 212, 213, 214, 215, 216, 217, 218, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 254, 255, 256, 257, 258, 259, 260, 261, 262, 263, 265, 267, 268, 269, 271, 272, 273, 274, 275, 277, 278, 279, 280, 281, 282, 283, 284, 285, 286, 287, 288, 291, 292, 293, 294, 295, 296, 297, 298, 299, 301, 302, 303, 304, 305, 306, 307, 308, 310, 311, 312, 314, 315, 316, 317, 318, 320, 321, 322, 324, 325, 326, 328, 329, 331, 335, 337, 338, 340, 341, 342, 343, 344, 345, 346, 347, 348, 349, 350, 351, 352, 353, 354, 355, 357, 359, 360, 362, 363, 364, 365, 366, 367, 368, 369, 370, 371, 372, 373, 374, 375, 376, 377, 378, 379, 380, 381, 383, 386, 387, 388, 389, 390, 391, 392, 393, 394, 395, 396, 398, 399, 400, 401, 402, 403, 404, 406, 407, 408, 409, 410, 411, 412, 413, 414, 415, 416, 417, 418, 419, 421, 422, 423, 424, 425, 426, 427, 428, 429, 430, 431, 432, 433, 434, 435, 436, 437, 438, 439, 440, 441, 442, 443, 444, 445, 446, 448, 449, 450, 451, 452, 453, 454, 455, 456, 457, 459, 461, 462, 463, 464, 465, 466, 467, 469, 470, 471, 472, 473, 474, 475, 476, 477, 478, 479, 480, 485, 487, 488, 491, 492, 493, 494, 495, 496, 497, 498, 499, 500, 501, 502, 503, 504, 505, 506, 507, 508, 509, 510, 511, 512, 514, 516, 517, 518, 519, 520, 521, 524, 525, 526, 527, 528, 529, 530, 531, 534, 535, 537, 538, 539, 540, 542, 543, 545, 546, 547, 548, 549, 550, 551, 553, 554, 555, 556, 557, 558, 559, 560, 561, 563, 564, 565, 567, 570, 571, 572, 573, 574, 575, 576, 577, 578, 579, 580, 581, 582, 583, 584, 585, 586, 587, 588, 589, 590, 591, 592, 593, 594, 595, 597, 598, 600, 604, 605, 606, 608, 609, 610, 611, 612, 613, 631, 633, 634, 635, 636, 637, 638, 639, 640, 641, 642, 643, 644, 645, 646, 647, 648, 649, 650, 651, 652, 653, 654, 655, 656, 657, 659, 661, 662, 669, 670, 672, 673, 674, 675, 676, 677, 678, 679, 680, 681, 682, 683, 684, 685, 686, 687, 688, 689, 690, 693, 694, 695, 696, 697, 698, 700, 701, 703, 704, 705, 706, 708, 709, 710, 711, 712, 713, 714, 715, 718, 720, 721, 723, 724, 725, 726, 727, 728, 729, 730, 731, 732, 733, 734, 735, 736, 737, 738, 739, 740, 741, 742, 743, 744, 745, 746, 747, 748, 750, 751, 752, 753, 755, 756, 757, 758, 759, 760, 761, 762, 765, 766, 768, 769, 770, 771, 772, 773, 774, 775, 776, 777, 778, 779, 780, 781, 782, 783, 784, 785, 786, 787, 788, 789, 790, 791, 792, 793, 794, 795, 796, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 840, 841, 842, 843, 844, 845, 846, 847, 848, 849, 850, 851, 852, 853, 854, 855, 857, 858, 859, 860, 861, 862, 863, 864, 865, 866, 870, 871, 872, 873, 874, 875, 877, 879, 880, 881, 882, 883, 884, 885, 886, 887, 888, 889, 893, 894, 895, 896, 897, 898, 899, 900, 901, 902, 903, 904, 905, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 973, 974, 975, 976, 977, 978, 979, 980, 981, 984, 985, 986, 987, 988, 989, 990, 991, 992, 993, 994, 995, 996, 997, 998, 999, 1000, 1002, 1003, 1004, 1005, 1006, 1007, 1008, 1009, 1010, 1011, 1012, 1013, 1014, 1015, 1016, 1017, 1018, 1021, 1022, 1023, 1024, 1026, 1027, 1028, 1029, 1030, 1031, 1033, 1034, 1035, 1036, 1037, 1038, 1039, 1040, 1041, 1042, 1043, 1044, 1045, 1046, 1047, 1048, 1050, 1051, 1052, 1053, 1055, 1057, 1058, 1059, 1060, 1062, 1063, 1064, 1065, 1066, 1067, 1068, 1070, 1071, 1072, 1073, 1074, 1075, 1076, 1077, 1078, 1079, 1081, 1082, 1083, 1084, 1085, 1086, 1087, 1088, 1089, 1090, 1091, 1092, 1093, 1094, 1095, 1096, 1097, 1098, 1099, 1100, 1101, 1102, 1103, 1104, 1105, 1106, 1107, 1108, 1109, 1111, 1113, 1114, 1115, 1116, 1117, 1118, 1119, 1120, 1121, 1122, 1123, 1124, 1125, 1126, 1127, 1128, 1129, 1130, 1131, 1132, 1133, 1134, 1135, 1136, 1137, 1138, 1139, 1140, 1141, 1142, 1143, 1144, 1145, 1146, 1147, 1148, 1149, 1150, 1151, 1152, 1153, 1154, 1155, 1157, 1158, 1159, 1161, 1162, 1166, 1167, 1168, 1169, 1170, 1171, 1172, 1173, 1174, 1175, 1177, 1178, 1179, 1180, 1181, 1182, 1184, 1185, 1186, 1188, 1189, 1190, 1191, 1192, 1193, 1194, 1195, 1197, 1198, 1199, 1202, 1203, 1204, 1205, 1208, 1209, 1210, 1211, 1213, 1214, 1215, 1217, 1218, 1219, 1220, 1221, 1222, 1223, 1224, 1225, 1226, 1227, 1228, 1230, 1231, 1232, 1233, 1234, 1235, 1236, 1237, 1238, 1239, 1240, 1241, 1242, 1243, 1244, 1246, 1247, 1248, 1249, 1250, 1251, 1252, 1253, 1254, 1255, 1256, 1257, 1258, 1259, 1261, 1262, 1263, 1264, 1265, 1266, 1267, 1269, 1270, 1271, 1272, 1273, 1274, 1275, 1276, 1277, 1278, 1279, 1280, 1281, 1282, 1283, 1284, 1285, 1286, 1287, 1288, 1289, 1290, 1291, 1292, 1293, 1294, 1295, 1296, 1297, 1298, 1301, 1302, 1303 or 1304 of the polypeptide of SEQ ID NO: 1.

In other preferred embodiments, the lactase variants of the present invention comprise an alteration, preferably a substitution, at one or more positions corresponding to the positions listed in any one of embodiments 5, 7, 9, 11, 13, 15, 17 19, 21, 23, 25, 27, 29, 31, 33, 35, 37, 39, 41, 43, 45, 47, 49, 51, 53, 55, 57, 59, 61, 63, 65, 67, 69 or 71 of the “PREFERRED EMBODIMENTS” section right before the “EXAMPLES” section of the present application.

Preferably, the lactase variants of the present invention comprise one or more of the following substitutions, wherein each position corresponds to a position in SEQ ID NO: 1: V1D, V1F, V1H, V1K, V1L, V1R, E2D, E2G, E2Q, E2V, D3A, D3H, D3I, D3N, D3S, D3V, D3W, A4C, A4G, A4H, A4I, A4L, A4M, A4P, T5A, T5D, T5F, T5K, T5R, T5S, T5V, R6A, R6D, R6F, R6G, R6H, R6L, R6M, R6P, R6S, R6W, S7A, S7D, S7I, S7L, S7N, S7P, S7T, S7W, S9G, S9H, S9P, S9R, S9W, T10G, T10K, T10L, T10P, T10R, T10S, T10W, T11L, T11P, Q12L, Q12R, Q12V, Q12Y, M13C, M13D, M13E, M13F, M13H, M13K, M13R, M13W, S14A, S14G, S14H, S14L, S14T, S14V, S14Y, S15C, S15F, S15I, S15K, S15L, S15P, S15R, S15T, S15V, S15W, S15Y, T16A, T16C, T16I, T16N, T16S, P17A, P17C, P17D, P17E, P17I, P17L, P17N, P17R, P17S, P17T, V19A, V19F, V19G, V19I, V19K, V19L, V19N, V19S, V19W, V20C, V20F, V20G, V20I, V20K, V20L, V20M, V20N, V20P, V20Q, V20R, V20T, V20W, Y21A, Y21C, Y21D, Y21F, Y21G, Y21H, Y21M, Y21P, Y21R, Y21T, S22A, S22E, S22F, S22G, S22L, S22M, S22N, S22R, S22T, S22W, S23A, S23C, S23D, S23L, S23M, S23R, A24F, A24G, A24L, A24R, A24T, A24W, V25D, V25E, V25F, V25G, V25H, V25K, V25L, V25M, V25Q, V25R, V25S, V25T, V25W, D26C, D26I, D26L, D26M, D26T, D26V, S27A, S27C, S27F, S27G, S27H, S27P, S27Y, K28C, K28G, K28I, K28L, K28R, K28S, K28V, K28W, Q29D, Q29F, Q29G, Q29L, Q29M, Q29R, Q29S, Q29V, Q29W, N30A, N30G, N30H, N30M, N30P, N30V, N30W, N30Y, R31E, R31G, R31I, R31M, R31V, T32M, T32Q, T32R, T32S, S33C, S33E, S33H, S33K, S33N, S33Q, S33R, S33V, D34C, D34E, D34F, D34G, D34H, D34L, D34S, D34W, D34Y, F35A, F35C, F35E, F35G, F35K, F35N, F35T, F35V, D36H, D36Q, A37N, A37Q, N38C, N38G, N38S, W39G, W39S, K40C, K40D, K40F, K40G, K40I, K40M, K40N, K40P, K40W, F41A, F41C, F41G, F41I, F41Q, F41S, F41Y, M42E, M42N, M42T, L43A, L43C, L43G, L43I, L43S, L43T, L43V, S44C, S44M, S44N, S44Y, D45A, D45L, D45P, D45V, V47K, V47R, Q48S, A49C, A49D, A49H, A49R, A49S, A49T, A49V, D51G, D51I, D51K, D51M, D51P, D51V, A53C, A53G, A53L, A53R, A53S, A53T, A53V, A53W, F54M, F54S, D55C, D55F, D55G, D55H, D55M, D55N, D55P, D55S, D55V, S57A, S57C, S57E, S57G, A58D, A58G, A58I, A58M, A58N, A58Q, A58R, A58T, W59D, W59I, W59K, W59L, W59N, W59P, W59V, Q60A, Q60C, Q60E, Q60F, Q60G, Q60K, Q60L, Q60M, Q60R, Q60S, Q60V, Q60Y, Q61K, Q61P, Q61S, V62G, V62N, V62S, V62T, V62W, D63G, D63L, D63P, D63S, D63V, L64E, L64G, H66C, H66L, H66R, H66T, H66W, H66Y, D67E, Y68P, Y68V, I70A, I70H, I70K, I70P, I70R, T71C, T71E, T71G, T71H, T71K, T71L, T71N, T71P, T71Q, T71R, T71S, K73A, K73D, K73F, K73G, K73Q, K73V, Y74G, Y74K, Y74T, Y74W, S75G, S75L, S75Q, S75R, S75V, Q76G, Q76I, Q76K, Q76M, Q76P, Q76S, Q76V, Q76Y, S77C, S77D, S77E, S77F, S77G, S77H, S77I, S77K, S77L, S77M, S77R, S77T, S77V, S77W, S77Y, N78C, N78E, N78F, N78K, N78Q, N78R, N78S, N78T, E79H, E79Q, E79S, E79T, E79W, A80K, E81A, E81Q, A83E, A83T, L85A, L85C, L85D, L85F, L85M, L85N, L85S, L85V, L85W, P86E, P86G, P86H, P86N, P86Q, P86R, P86V, P86W, P86Y, G87A, G87D, G87E, G87N, G87Q, G88A, G88F, G88I, G88M, G88Q, G88S, T89C, T89G, T89H, T89K, T89L, T89M, T89N, T89P, T89W, T89Y, G90A, G90C, G90D, G90L, G90S, G90T, G90V, W91E, W91L, W91P, W91Q, W91R, W91S, W91Y, Y92F, Y92H, Y92I, Y92M, Y92N, Y92S, Y92T, Y92V, Y92W, R93A, R93D, R93F, R93H, R93I, R93L, R93N, R93T, R93V, R93Y, K94A, K94C, K94G, K94R, K94S, K94T, K94V, S95A, S95C, S95D, S95E, S95G, S95I, S95L, S95Q, S95R, F96A, F96C, F96I, F96K, F96L, F96M, F96P, F96S, F96V, F96W, T97F, T97S, T97V, I98C, I98H, I98S, I98W, R100T, D101A, D101P, D101V, L102A, L102G, L102M, L102P, L102S, G104C, K105D, K105Q, K105R, K105W, K105Y, R106K, R106P, R106V, R106W, I107A, I107F, I107G, I107Q, I107S, A108E, A108S, A108V, I109M, I109T, N110A, N110F, N110S, N110T, N110V, N110W, F111A, F111C, F111L, F111Q, F111V, D112A, D112F, D112G, D112T, G113A, G113S, V114F, V114G, V114M, V114R, Y115E, M116A, M116C, M116D, M116W, M116Y, N117K, N117R, N117T, N117W, A118K, A118P, A118Y, T119A, T119G, T119L, V120A, V120K, W121C, W121D, W121R, W121T, W121V, W121Y, F122A, F122M, F122S, F122Y, N123P, G124E, G124M, G124Q, G124R, V125D, V125E, V125I, K126E, K126V, G128A, G128D, T129E, T129V, H130A, H130C, H130Q, H130S, H130T, P131K, P131L, P131S, Y132C, Y132E, Y132S, G133E, S135E, S135P, S135V, P136R, P136Y, F137A, F137C, F137D, F137G, F137L, F137P, S138A, S138D, S138G, S138H, S138L, S138M, S138R, S138V, F139A, F139E, F139Q, F139W, D140G, D140L, D140V, L141G, L141T, T142E, T142S, T142V, K146A, G148H, G148K, G148T, G149E, G149H, G149I, G149M, G149Q, G149Y, E150A, E150C, E150G, E150L, E150N, E150R, N151L, I153A, I153Y, V154E, V154I, V154K, V154L, V154M, V154S, V155F, V157A, V157G, V157L, V157P, V157Q, V157S, E158G, E158H, E158K, E158Q, E158V, N159D, N159H, N159S, N159T, R160G, L161E, L161K, L161M, L161S, L161W, P162F, P162G, P162N, P162T, P162W, S164A, R165H, W166S, Y167A, Y167C, S168C, G169A, G169C, G169D, G169S, S170L, S170Q, G171C, G171F, G171T, I172G, I172K, I172P, I172Q, Y173A, Y173H, Y173M, Y173P, Y173S, Y173W, R174E, R174K, D175E, D175Y, V176E, V176K, V176T, T177A, T177C, T177E, T177K, T177L, L178I, L178Q, L178W, T179A, T179C, T179D, T179H, T179I, T179K, T179L, T179N, T179P, T179S, V180A, V180C, V180D, V180E, V180G, V180M, T181A, T181D, T181F, T181K, T181R, D182F, D182L, D182S, G183W, V184F, V184H, V184P, V184Q, V184R, V184S, V184W, H185G, H185L, H185R, V186A, V186E, V186G, V186N, G187A, G187D, G187H, N188E, N188R, N188S, N188V, N188W, N189A, N189E, G190C, G190F, G190H, G190Q, G190V, V191C, V191T, V191W, V191Y, I193N, I193Q, I193T, I193V, K194A, K194I, K194L, K194R, T195A, T195E, T195M, T195S, T195W, P196A, P196H, P196I, P196M, P196S, P196W, S197A, S197C, S197E, S197K, S197L, S197P, L198E, L198F, L198H, L198I, L198K, L198R, L198V, L198W, A199E, A199F, A199K, A199P, A199R, A199T, Q201C, Q201E, Q201I, Q201K, Q201M, Q201V, N202A, N202D, N202F, N202G, N202K, N202L, N202M, N202Q, N202R, N202S, N202T, N202W, G203C, G203K, G203M, G203Q, G203R, G203S, G203V, G203W, G203Y, G204A, G204C, G204D, G204K, G204R, G204S, G204Y, N205E, N205G, N205H, N205L, N205P, N205W, N205Y, V206A, V206C, V206D, V206F, V206G, V206I, V206K, V206Q, V206R, V206S, V206T, T207A, T207C, T207G, T207I, T207K, T207L, T207M, T207N, T207Q, T207R, T207W, M208A, M208S, M208T, N209C, N209D, N209G, N209K, N209L, N209Q, N209R, N209V, L210A, L210C, L210F, L210G, L210H, L210I, L210Q, L210R, L210S, L210T, L210V, T211A, T211D, T211E, T211F, T211K, T211N, T211Q, T211R, T211S, T212A, T212C, T212E, T212F, T212G, T212H, T212K, T212L, T212M, T212S, T212W, K213A, K213C, K213D, K213F, K213I, K213L, K213M, K213N, K213Q, K213R, K213S, K213T, K213V, K213Y, V214A, V214C, V214T, V214W, A215D, A215E, A215F, A215I, A215K, A215L, A215Q, A215R, A215S, A215V, N216D, N216T, N216V, D217F, D217G, D217L, D217M, D217T, D217V, T218D, T218G, T218H, K219A, K219C, K219F, K219H, K219M, A220C, A220G, A220I, A220L, A220M, A220T, A220V, A220W, A221C, A221D, A221E, A221L, A221N, A221R, A221V, A221Y, A222D, A222I, A222L, A222P, A222R, A222W, A222Y, N223A, N223E, N223F, N223G, N223K, N223L, N223M, N223R, N223S, N223T, N223V, N223W, I224G, I224Q, T225A, T225G, T225L, L226C, L226M, L226Q, K227T, Q228N, Q228R, T229A, T229C, T229D, T229G, T229H, T229M, T229N, T229Q, T229R, T229V, V230F, V230L, V230M, V230Q, V230R, V230S, F231A, F231E, F231G, F231I, F231K, F231L, F231Q, F231S, F231V, F231W, F231Y, P232G, P232H, P232L, P232M, P232R, P232S, P232T, P232V, P232W, P232Y, K233A, K233C, K233E, K233F, K233G, K233L, K233P, K233R, K233S, K233V, K233W, K233Y, G234A, G234C, G234D, G234E, G234K, G234L, G234Q, G234R, G234V, G234W, G234Y, G235C, G235F, G235H, G235l, G235K, G235M, G235Q, G235R, G235T, G235W, G235Y, K236A, K236D, K236E, K236G, K236L, K236M, K236P, K236R, K236S, K236T, K236W, K236Y, T237D, T237F, T237I, T237K, T237M, T237Q, T237R, T237S, T237V, T237Y, D238A, D238E, D238F, D238G, D238H, D238I, D238K, D238L, D238M, D238N, D238P, D238Q, D238R, A239C, A239E, A239G, A239I, A239K, A239T, A240C, A240E, A240L, A240P, A240Q, A240T, A240V, A240W, A240Y, I241T, G242K, G242L, G242M, G242P, G242T, G242Y, T243I, T243M, T243R, T243V, V244A, V244E, V244G, V244L, V244R, T245E, T245G, T245L, T245M, T245N, T245Q, T245R, T245S, T246D, T246E, T246G, T246K, T246V, A247D, A247E, A247K, A247N, A247P, A247Q, A247R, A247S, A247V, A247W, S248A, S248E, S248F, S248H, S248I, S248L, S248Q, S248T, S248Y, K249A, K249D, K249G, K249H, K249I, K249L, K249M, K249N, K249P, K249Q, K249S, K249T, K249V, K249Y, S250H, S250M, S250W, I251F, I251L, I251V, I251W, I251Y, A252C, A252E, A252F, A252H, A252I, A252P, A252R, A252S, A252W, A252Y, G254D, G254F, G254I, G254L, G254M, G254Q, G254R, G254W, A255C, A255F, A255K, A255L, A255M, A255S, A255T, A255W, A255Y, S256A, S256C, S256F, S256G, S256K, S256L, S256M, S256N, S256Q, S256R, S256V, S256W, S256Y, A257D, A257G, A257I, A257N, A257T, A257V, D258A, D258L, D258M, D258W, V259E, V259L, V259S, V259T, T260A, T260D, T260G, T260I, T260K, T260V, S261A, S261D, S261H, S261R, S261W, S261Y, T262D, T262E, T262F, T262G, T262H, T262L, T262P, T262W, I263A, I263C, I263G, I263L, I263S, I263V, T264F, T264G, T264K, T264L, T264M, T264P, T264Q, T264R, T264S, T264Y, A265G, A265I, A265K, A265R, A265S, A265V, A266D, A266E, A266G, A266K, A266L, A266M, A266P, A266Q, A266S, A266T, S267A, S267D, S267K, S267M, S267N, S267P, S267Q, S267R, S267V, P268F, P268G, P268M, P268R, P268V, P268W, P268Y, K269G, K269R, K269V, K269Y, L270D, L270M, L270N, L270R, L270V, W271T, S272E, S272G, S272K, S272L, S272N, S272T, S272W, I273K, I273L, I273P, I273R, I273S, I273W, K274D, K274P, K274Q, K274R, N275K, N275M, N275V, N275W, N277F, N277K, N277R, L278A, L278G, L278H, L278I, L278K, L278M, L278P, L278Q, L278R, L278S, L278V, Y279M, Y279T, Y279W, T280A, T280D, T280E, T280F, T280H, T280M, T280Q, V281A, V281I, V281L, V281Q, R282E, R282F, R282H, R282I, R282K, R282N, R282S, R282T, R282V, R282W, T283M, T283R, T283V, E284A, E284D, E284F, E284H, E284L, E284M, E284N, E284Q, E284R, E284Y, V285H, V285I, V285T, L286A, L286C, L286D, L286F, L286N, L286R, L286T, L286W, L286Y, N287I, N287L, G288F, G288L, G288S, V291C, V291D, V291F, V291G, V291H, V291L, V291P, V291S, V291T, V291Y, L292A, L292D, L292E, L292H, L292Q, L292S, L292V, D293C, D293E, D293F, D293I, D293S, D293V, D293W, T294C, T294G, T294M, T294Q, T294S, Y295F, Y295G, Y295I, Y295L, Y295R, Y295S, Y295W, D296F, D296H, D296K, D296L, D296N, D296R, D296V, T297G, T297I, T297Q, T297R, T297S, E298G, E298I, E298L, E298M, E298R, E298T, Y299F, Y299S, F301C, F301L, R302E, R302I, R302K, R302M, R302N, R302T, W303A, W303C, W303D, W303F, W303S, W303T, T304D, T304E, T304I, T304K, T304P, T304S, T304W, G305E, G305F, G305L, G305M, G305N, G305P, G305T, G305W, F306D, F306K, F306L, F306S, D307A, D307E, D307F, D307G, D307L, D307M, D307N, D307Q, D307S, D307V, D307W, A308C, A308E, A308G, A308I, A308M, A308Y, T309C, T309D, T309E, T309I, T309K, T309M, T309S, T309V, S310A, S310C, S310G, S310H, S310I, S310L, S310M, S310N, S310R, S310V, G311E, G311F, G311L, G311Q, G311R, G311V, F312H, F312L, F312R, F312V, L314A, L314C, L314T, L314V, L314Y, N315S, G316A, G316C, G316N, G316R, G316S, G316W, G316Y, E317D, E317H, E317I, E317K, E317V, E317W, E317Y, K318C, K318E, K318F, K318G, K318H, K318L, K318M, K318N, K318P, K318Q, K318V, K320C, K320E, K320F, K320I, K320L, K320R, K320T, K320V, K320W, L321C, L321G, L321H, L321M, L321V, L321Y, K322C, K322F, K322I, K322N, K322P, K322Q, K322R, K322S, V324E, V324G, V324T, S325D, S325G, S325T, M326E, M326G, M326S, M326T, M326V, M326Y, H328C, H328D, H328F, H328G, H3281, H328L, H328M, H328R, H328T, D329S, D329T, G331V, A335C, A335G, A335L, A337D, A337E, A337G, A337L, A337Q, N338P, N338R, N338V, R340A, R340C, R340L, A341M, A341S, I342A, I342G, I342K, I342P, E343A, E343H, E343N, E343R, E343S, E343T, E343Y, R344G, R344L, R344Y, Q345F, Q345G, Q345K, Q345N, Q345S, V346A, V346C, V346F, V346I, V346L, V346S, E347A, E347C, E347D, E347F, E347I, E347R, E347S, I348A, I348D, I348G, I348M, I348Q, I348R, L349A, L349E, L349Q, L349S, L349V, Q350G, Q350N, Q350W, K351N, K351R, K351V, K351W, M352G, M352L, M352T, M352W, G353K, V354E, V354G, V354S, V354W, N355H, N355M, N355R, N355W, I357T, T359E, T359L, T360V, N362G, N362S, N362T, P363A, P363D, P363I, P363L, P363M, P363S, P363V, A364C, A364E, A364F, A364G, A364I, A364M, A364P, A364V, A364W, A365C, A365E, A365I, A365P, A365V, A365W, K366A, K366D, K366E, K366I, K366L, K366M, K366P, K366S, K366V, A367C, A367I, A367N, A367Q, L368A, L368E, L368Q, L368S, L368V, L368Y, I369A, I369D, I369E, I369G, I369K, I369M, I369R, I369V, I369W, D370C, D370L, D370Q, D370R, D370S, D370T, V371D, V371F, V371G, V371I, V371L, V371Q, V371S, C372P, N373G, N373L, N373R, E374K, E374L, E374R, K375D, K375I, K375N, K375Q, K375S, G376A, G376S, V377A, V377M, V377T, L378I, L378P, L378W, L378Y, V379A, V379C, V379M, V379N, V380M, V380P, V380S, E381A, E381C, E381G, E381L, E381Q, E381T, V383A, V383K, V383L, M386G, M386N, M386Q, M386S, M386V, W387H, W387L, N388A, N388E, N388L, N388R, R389A, R389C, R389E, R389K, R389M, R389N, R389Q, R389S, R389T, R389V, S390C, S390D, S390G, S390H, S390N, S390P, S390Q, S390T, S390V, K391E, N392D, G393A, G393E, G393N, G393R, G393S, G393V, N394L, T395A, T395C, T395F, T395H, T395I, T395M, T395N, T395Q, T395S, T395W, E396K, E396L, E396M, E396V, E396W, Y398M, Y398N, G399S, K400A, K400C, K400D, K400E, K400M, K400N, K400P, K400Q, K400S, K400T, K400V, W401F, W401H, W401K, W401L, W401M, W401R, F402T, F402W, F402Y, G403A, G403D, G403H, G403K, G403P, G403Q, G403S, G403T, G403V, G403Y, Q404F, Q404H, Q404L, Q404M, Q404P, Q404R, Q404S, Q404V, A405C, A405E, A405H, A405K, A405P, A405R, A405T, A405V, I406C, I406D, I406N, A407C, A407G, A407M, A407Q, A407S, A407T, A407W, G408D, G408I, G408M, G408N, G408W, D409N, D409W, N410C, N410R, N410Y, A411E, A411N, A411R, A411S, A411V, V412M, V412S, L413D, L413E, L413F, L413I, L413P, L413T, G414A, G414C, G414M, G414N, G414R, G414T, G414W, G415A, G415Q, G415R, D416I, D416M, D416R, D416T, D416Y, K417C, K417F, K417G, K417R, K417T, D418L, D418P, D418R, D418Y, E419M, E419R, E419W, T420E, T420F, T420G, T420K, T420R, T420V, W421L, W421Q, W421S, A422P, A422T, A422V, K423D, K423L, K423M, K423R, F424C, F424L, F424N, F424T, D425E, L426C, L426M, L426Q, T427D, T427F, T427G, T427K, T427M, T427P, T427Q, T427R, T427S, T427W, S428F, S428K, S428W, T429D, T429P, I430C, I430D, I430E, I430L, I430M, I430Q, I430S, I430T, I430W, N431D, N431E, N431G, N431L, N431M, N431R, N431V, N431Y, R432A, R432E, R432F, R432G, R432N, R432Q, R432V, R432Y, D433C, D433G, D433H, D433I, D433P, D433Q, D433W, R434L, R434M, R434N, R434P, R434S, R434T, R434V, N435E, N435F, N435H, N435K, N435L, N435M, N435R, N435V, N435W, A436C, A436D, A436E, A436G, A436I, A436L, A436M, A436Q, A436S, P437A, P437D, P437K, P437L, P437Q, P437R, P437S, P437V, P437W, S438G, V439C, V439E, V439G, V439I, V439K, V439Q, V439T, V439Y, I440C, I440D, I440F, I440K, I440P, I440R, I440S, I440T, I440V, I440W, M441A, M441E, M441G, M441Q, M441R, M441T, M441V, W442E, W442G, W442M, W442P, W442Q, W442R, S443C, S443D, S443G, S443M, S443Q, S443Y, L444C, L444D, L444E, L444F, L444G, L444H, L444K, L444Q, L444V, L444W, G445A, G445C, G445V, N446D, N446T, M448A, M448C, M448D, M448E, M448I, M448L, M448P, M448Q, M448S, M448V, M448W, M449D, M449E, M449F, M449T, M449V, M449W, M449Y, E450F, E450S, E450T, E450V, E450W, G451C, G451F, G451L, G451P, G451V, G451W, I452G, I452K, I452L, I452M, I452Q, I452S, I452V, S453C, S453F, S453G, S453H, S453L, S453M, S453N, S453P, S453Q, S453R, S453V, G454L, G454W, S455A, S455E, S455K, S455M, S455P, S455R, S455V, S455W, V456A, V456D, V456E, V456F, V456K, V456L, V456W, S457E, S457H, S457K, S457L, S457M, S457P, S457Q, S457T, S457V, G458A, G458C, G458D, G458F, G458L, G458P, G458Q, G458S, G458V, G458W, F459A, F459C, F459E, F459G, F459N, F459R, F459S, F459T, F459W, P460C, P460M, P460Q, P460W, P460Y, A461D, A461G, A461M, A461N, A461Q, A461S, A461V, A461Y, T462C, T462E, T462F, T462L, T462M, T462S, S463G, S463K, S463Q, S463R, S463T, S463V, A464E, A464H, A464L, A464M, A464P, A464V, A464W, K465C, K465F, K465G, K465L, K465Q, K465R, K465V, K465W, K465Y, L466A, L466C, L466D, L466E, L466F, L466G, L466M, L466P, L466Q, L466S, L466V, L466Y, V467A, V467C, V467D, V467E, V467G, V467T, V467W, A468D, A468E, A468F, A468K, A468L, A468P, A468S, A468V, A468W, W469A, W469C, W469D, W469G, W469L, W469M, W469R, W469V, W469Y, T470E, T470L, T470M, T470Q, K471F, K471G, K471Q, K471W, K471Y, A472G, A472Y, A473E, A473M, A473P, D474A, D474C, D474E, D474K, D474M, D474R, D474W, S475E, S475F, S475Q, S475T, S475V, T476C, T476L, T476S, R477A, R477C, R477G, R477L, P478A, P478D, P478G, P478L, P478V, M479G, M479I, M479R, M479W, T480C, T480G, T480Q, K485E, K485R, K487A, K487C, K487F, K487G, K487N, K487S, K487W, A488C, A488G, A488H, A488L, A488N, A488S, A488V, N491A, N491E, N491W, E492A, E492W, S493E, S493G, S493H, S493L, S493M, S493Q, S493V, N494A, N494I, N494M, N494R, N494V, T495K, T495R, T495V, T495W, M496A, M496F, M496T, G497D, D498A, D498C, D498M, D498S, N499K, N499R, N499T, N499Y, L500A, L500E, L500N, L500V, T501C, T501G, T501M, A502L, A502Q, N503A, N503E, N503M, N503S, G504H, G504K, G504P, G505A, G505D, G505E, G505H, G505L, G505N, G505R, G505S, G505V, V506C, V506D, V506E, V506G, V506I, V506L, V506P, V506R, V506S, V506T, V506W, V507A, V507F, V507G, V507L, V507N, V507P, V507R, V507S, V507T, G508C, G508E, T509A, T509D, T509E, T509I, T509K, T509M, T509Q, T509S, T509V, T509Y, N510A, N510F, N510I, N510Q, Y511A, Y511K, S512C, S512E, S512F, S512G, S512I, S512M, S512Q, S512T, S512V, S512Y, D513C, D513G, D513K, D513L, D513M, D513P, D513Q, D513R, D513W, G514F, G514L, G514N, G514P, G514R, A515C, A515D, A515E, A515F, A515G, A515K, A515P, A515R, A515S, N516C, N516E, N516G, N516I, N516M, N516Q, N516S, N516T, N516V, Y517G, Y517I, Y517N, D518Q, D518Y, K519C, K519E, K519F, K519G, K519I, K519L, K519M, K519N, K519Q, K519S, K519T, I520A, I520C, I520G, I520H, I520M, I520N, I520Q, I520S, I520T, I520W, I520Y, R521A, R521C, R521K, R521N, R521Q, R521V, T522G, T522I, T522N, T523A, T523M, H524R, H524V, P525D, P525G, P525R, P525T, P525V, S526G, S526I, S526W, W527A, W527E, W527G, W527H, W527N, W527R, W527S, W527V, A528C, A528E, A528G, A528I, A528L, I529F, I529G, I529L, I529Y, Y530A, Y530M, G531E, G531S, G531T, T534A, T534I, T534Q, A535E, A535G, A535I, A535M, A537D, A537M, A537P, A537S, I538H, I538M, N539G, N539W, S540E, S540G, S540M, G542E, G542Q, G542S, G542T, I543V, I543W, N545G, N545I, N545Q, N545S, R546A, R546C, R546L, R546P, R546S, T547A, T547D, T547H, T547K, T547N, T547S, T548D, T548E, T548F, T548K, T548L, T548P, T548W, G549D, G549F, G549P, G549W, G550Q, G550R, G550S, A551D, A551I, A551Q, S553C, S553F, S553N, S553P, S553R, S553T, S553V, S554F, S554N, S554R, S554T, S554V, S554W, D555E, D555P, D555S, K556A, K556C, K556R, K556W, Q557F, Q557R, Q557S, L558E, L558H, L558I, L558P, T559A, T559G, T559I, T559Q, T559V, T559Y, S560P, S560V, Y561R, N563R, N563S, S564A, S564C, S564F, S564W, A565C, A565D, A565E, A565F, A565G, A565I, A565L, A565M, A565R, A565S, A565T, A565V, A565W, G567N, G567Q, G567V, A570G, A570K, A570L, A570M, A570S, A570T, A570W, V571W, A572S, A572W, S573G, S573K, S573Y, S574K, S574Q, S574V, S574W, A575D, A575M, A575V, W576A, W576F, W576V, W576Y, Y577G, Y577I, Y577L, Y577R, D578E, D578M, D578N, D578T, V579E, V579G, V579L, V579T, V580A, V580D, V580E, V580K, V580L, V580S, Q581F, Q581G, Q581P, Q581R, Q581S, Q581T, Q581Y, R582A, R582D, R582G, R582I, R582L, R582Y, D583V, D583W, F584E, F584I, F584W, V585I, V585M, V585Q, A586C, A586D, A586H, A586K, G587A, G587C, T588C, T588D, T588G, T588I, T588L, T588M, T588P, Y589A, Y589I, Y589Q, Y589V, Y589W, V590A, V590H, V590I, W591F, T592C, T592L, T592Q, T592S, G593C, G593I, F594C, F594I, F594L, F594M, D595E, D595Q, D595S, L597C, L597D, L597E, L597T, G598N, P600A, P600E, P600G, P600S, N604E, N604S, G605R, T606S, G607A, G607Q, G607S, S608E, S608I, S608M, S608N, S608Q, S608T, S608V, G609A, G609H, G609L, G609N, G609R, G609S, A610D, A610F, A610M, A610T, V611K, G612N, G612T, G612V, S613A, W614A, W614F, W614P, P615L, P615S, P615V, S616A, S616W, N619A, N619I, N619L, N619S, S620G, Y621W, I624A, I624V, V625A, V625E, V625F, V625M, V625Y, T627F, T627G, T627K, T627Q, A628C, A628D, A628N, G629T, F630A, F630C, F630D, F630G, F630S, F630Y, P631A, P631D, P631G, P631H, P631S, P631V, P631Y, K632C, K632D, K632G, K632T, D633G, T634A, T634E, T634F, T634S, T634V, Y635R, Y636K, F637C, F637G, F637I, F637S, F637T, F637V, Y638A, Y638W, Q639D, Q639N, Q639R, S640C, S640D, Q641T, W642I, N643R, D644C, D644G, D644Y, D645S, D645V, V646C, V646L, V646N, V646R, V646S, H647G, H647V, T648C, L649V, H650E, H650F, H650R, I651F, I651T, I651V, L652C, L652D, L652V, L652W, P653Q, A654D, A654K, A654M, A654R, W655F, W655R, N656A, N656K, N656V, E657K, E657R, E657V, V659D, V659N, A661E, A661G, A661H, A661K, A661L, A661M, A661Q, A661W, K662H, K662S, K662V, K662W, K662Y, N667L, N667R, P669A, P669E, P669F, P669L, P669R, P669T, P669W, V670C, V672L, Y673E, Y673G, Y673I, Y673R, Y673S, Y673W, T674C, T674D, T674G, T674H, T674M, T674Q, D675A, D675E, D675P, D675Q, D675S, D675V, D675Y, A676C, A676E, A676G, A676K, A676L, A676P, A676S, A676W, A677E, A677G, A677L, A677R, A677T, A677V, A677Y, K678A, K678C, K678T, K678V, V679G, V679Q, V679S, V679T, V679Y, K680A, K680E, K680G, K680H, K680I, K680L, K680N, K680Q, K680S, K680V, K680W, L681E, L681F, L681G, L681M, L681S, L681T, Y682D, Y682E, Y682I, Y682M, Y682S, Y682V, Y682W, F683H, F683L, F683M, F683Q, F683R, F683W, T684A, T684D, T684G, T684L, T684R, T684S, T684V, P685A, P685E, P685I, P685L, P685W, K686A, K686E, K686I, K686M, K686T, K686V, G687A, G687K, G687N, G687P, G687Q, G687R, S688C, S688D, S688E, S688G, S688K, S688L, S688P, S688T, T689A, T689D, T689E, T689G, T689L, T689P, T689S, T689W, T689Y, E690D, E690M, E690P, E690T, E690V, K691E, K691H, K691N, K691P, K691R, K691S, R692G, R692H, R692I, R692L, R692P, R692S, R692T, R692V, R692W, L693A, L693M, L693P, I694L, I694W, G695C, G695K, G695L, G695R, G695W, E696A, E696L, E696R, K697A, K697E, K697G, K697R, K697V, K697W, S698C, S698D, S698E, S698I, S698L, S698M, S698P, S698Q, S698R, S698T, T700A, T700C, T700D, T700E, T700G, T700K, T700Y, K701A, K701D, K701E, K701G, K701H, K701L, K701M, K701P, K701S, K701W, T703E, T703I, T703K, T703W, T704M, T704R, T704Y, A705C, A705D, A705E, A705K, A705N, A705P, A705R, A705V, A705W, A706C, A706G, A706T, A706W, A706Y, Y708C, Y708F, Y708G, Y708K, Y708L, Y708P, Y708T, T709M, Y710C, Y710D, Y710E, Y710G, Y710M, Y710N, Y710T, Y710V, Y710W, Q711A, Q711D, Q711G, Q711L, Q711M, Q711T, Q711Y, V712G, V712M, V712P, V712Q, V712R, Y713A, Y713E, Y713G, Y713L, Y713Q, E714D, E714H, E714I, E714K, E714M, E714N, E714R, E714S, E714V, E714Y, G715K, A716C, A716G, A716L, A716M, A716P, A716R, A716T, A716V, D717C, D717G, D717L, D717S, K718A, K718L, K718Q, K718T, K718W, K718Y, D719E, D719L, D719P, D719S, D719T, D719V, S720A, S720M, S720R, S720Y, T721D, T721H, T721K, T721L, T721N, T721P, T721Q, T721S, T721V, T721W, T721Y, A722Q, A722T, A722V, H723G, H723P, K724A, K724G, K724R, N725A, N725D, N725I, M726A, M726D, M726E, M726K, M726Q, M726V, M726W, Y727L, Y727T, L728C, L728K, L728Q, L728S, L728T, L728W, T729A, T729E, T729M, W730A, N731A, N731E, N731Q, N731S, N731Y, V732G, V732P, V732R, V732W, P733A, P733R, P733W, W734D, W734G, W734R, W734V, A735Q, A735R, A735S, A735T, A735W, E736S, G737F, G737K, G737N, G737Y, T738G, T738L, T738S, I739E, I739K, I739M, I739W, I739Y, S740D, S740E, S740F, S740H, S740L, A741I, A741P, A741S, A741V, E742D, E742M, E742Q, E742V, A743C, A743E, A743H, A743I, A743L, Y744A, Y744E, Y744I, Y744K, Y744L, Y744R, D745C, D745F, D745N, D745R, E746A, E746C, E746K, E746T, N747E, N747F, N747G, N747P, N747R, N748S, R749H, R749M, R749S, R749T, L750G, L750M, L750P, L750Q, L750S, I751C, I751H, I751Q, I751S, P752A, P752C, P752H, P752L, P752S, P752V, P752Y, E753Q, G754H, G754I, G754P, G754R, S755C, S755I, S755T, T756E, T756N, T756P, T756Q, T756S, T756W, E757A, G758A, G758V, N759D, N759R, N759S, N759V, A760G, A760N, A760P, A760Q, S761A, S761K, S761Q, V762D, V762G, V762K, V762W, T765A, T765P, T765R, T765W, G766M, G766S, A768H, A769F, A769G, A769I, A769N, A769V, A769Y, K770H, L771A, L771I, K772A, K772C, K772P, K772V, K772W, A773C, A773G, A773H, A773M, A773R, A773S, A773V, D774A, D774R, A775L, A775V, A775W, D776I, D776L, D776R, D776S, D776V, R777D, R777E, R777G, R777H, R777P, R777S, R777T, K778A, K778C, K778F, K778G, K778L, K778N, K778R, T779C, T779I, I780V, T781A, T781C, T781E, T781F, T781G, T781M, T781P, T781R, T781Y, A782C, A782E, A782K, A782N, A782P, A782Q, A782Y, D783A, D783C, D783E, D783R, G784A, G784F, G784L, G784S, G784T, K785C, K785I, K785S, K785V, K785W, K785Y, D786S, D786V, L787D, L787K, L787P, L787T, L787Y, S788A, S788G, S788I, Y789C, Y789D, Y789I, Y789V, I790A, I790C, I790F, I790R, I790V, E791A, E791D, E791F, E791M, E791S, E791T, E791V, E791W, V792C, V792G, V792L, V792S, V792Y, D793C, D793F, D793H, D793K, D793N, D793Y, V794C, V794D, V794L, V794Q, V794T, V794W, T795P, T795Y, D796M, D796Q, D796S, D796T, A797H, A797K, N798G, N798I, N798P, N798Q, G799D, G799K, G799L, G799M, G799Q, G799Y, H800A, H800F, H800G, H800L, H800S, H800V, I801C, I801E, I801W, V802E, V802I, V802P, V802S, V802Y, P803A, P803F, P803G, P803K, P803S, P803Y, D804E, D804G, D804K, D804N, D804S, A805C, A805F, A805G, A805I, A805N, A805P, A806F, A806I, A806Q, N807F, N807Q, N807V, N807W, R808C, R808F, R808G, R808I, R808N, R808P, R808Q, V809A, V809C, V809L, V809M, V809P, T810L, T810P, T810Q, T810R, T810Y, F811L, F811Y, D812E, D812F, D812I, D812Q, V813F, V813H, V813T, V813W, V813Y, K814G, K814H, K814I, K814L, K814P, G815A, G815F, G815M, G815P, G815V, A816C, A816D, A816F, A816I, A816N, A816V, A816W, G817C, G817H, G817I, G817N, G817S, K818D, K818F, K818L, K818Q, K818R, K818S, K818V, K818W, K818Y, L819F, L819W, V820C, V820F, V820I, V820K, V820R, V820W, G821A, G821C, G821E, G821F, G821I, G821K, G821M, G821N, G821V, G821Y, V822A, V822D, V822E, V822T, D823E, N824A, N824C, N824G, N824Q, G825A, S826A, S826F, S826G, S826I, S826L, S826R, S826W, S827C, S827Q, P828C, P828G, P828I, P828L, P828Y, D829C, D829I, D829S, D829T, D829V, H830E, H830G, H830M, H830P, H830Q, H830R, H830V, D831A, D831F, D831G, D831I, D831M, D831P, D831R, D831V, S832E, S832F, S832G, S832L, S832M, S832P, S832R, S832V, S832W, Y833C, Y833D, Y833E, Y833I, Y833K, Y833N, Y833P, Y833V, Q834F, Q834G, Q834M, A835D, A835E, A835F, A835H, A835K, A835W, D836C, D836E, D836H, D836Q, D836R, D836S, D836T, D836V, D836W, D836Y, N837D, N837F, N837G, N837H, N837L, N837P, N837T, R838D, R838F, R838G, R838K, R838M, R838N, R838S, R838W, K839A, K839C, K839D, K839E, K839G, K839L, K839N, K839P, K839R, A840G, A840I, A840P, A840V, F841C, F841D, F841I, F841K, F841W, S842A, S842L, S842M, G843A, G843C, K844A, K844G, K844L, K844W, K844Y, V845N, V845W, L846G, L846I, L846M, L846S, A847L, A847T, I848M, V849A, V849L, V849S, V849T, Q850C, Q850G, Q850I, Q850L, Q850T, Q850V, Q850Y, S851C, S851D, S851E, S851L, S851T, T852D, T852G, T852L, K853N, K853P, K853Q, K853V, E854C, E854I, E854M, E854R, A855K, A855V, A855Y, E857P, E857V, I858D, I858E, I858F, I858G, I858K, I858M, I858P, I858Q, I858Y, T859V, V860T, V860Y, T861F, T861I, T861Q, T861V, T861W, A862C, A862P, A862V, K863F, K863I, K863L, K863N, K863W, A864E, A864H, A864K, A864L, D865A, D865G, G866H, G866R, L867W, S870E, S870M, S870R, S870T, T871P, V872C, V872G, K873G, K873Y, I874G, A875R, T877A, V879P, V879S, P880S, G881W, T882A, T882M, T882R, S883L, T884A, E885V, K886E, K886L, K886V, K886W, T887A, T887D, T887F, T887G, T887N, T887R, T887V, V888A, V888D, V888G, R889G, Y892D, Y892P, Y892R, Y893E, Y893G, S894D, S894G, R895M, N896M, Y897V, Y898T, V899G, K900E, K900G, T901G, T901Q, T901R, T901V, T901Y, G902A, G902D, G902F, G902L, G902P, G902Q, G902R, G902S, G902W, N903D, N903I, K904D, K904E, K904M, K904N, K904R, K904S, K904V, K904W, P905A, P905C, P905R, P905V, P905W, P905Y, I906A, I906D, I906N, I906S, I906T, I906W, I906Y, L907F, L907S, L907Y, P908C, P908D, P908G, P908I, P908L, P908M, P908T, S909E, S909F, S909G, S909W, S909Y, D910C, D910I, D910S, D910W, V911A, V911S, E912A, E912K, E912L, E912T, E912V, V913G, V913Q, V913R, V913W, R914A, R914E, R914F, R914I, R914K, R914V, R914W, R914Y, Y915A, Y915C, Y915G, Y915I, Y915M, Y915Q, Y915V, S916G, D917C, D917E, D917F, D917L, D917M, D917Q, D917S, G918E, G918H, G918T, G918V, G918W, T919D, T919K, T919Q, T919W, T919Y, S920C, S920E, S920M, S920P, S920R, S920V, S920W, D921C, D921P, D921Q, D921V, R922A, R922G, R922M, R922V, R922W, Q923A, Q923C, Q923E, Q923L, Q923M, Q923V, Q923W, N924A, N924L, N924P, N924Q, N924S, N924W, V925A, V925C, V925E, V925G, V925K, V925N, V925S, V925W, T926G, T926P, T926R, T926S, T926V, T926W, W927C, W927G, W927P, D928A, D928E, D928H, D928L, D928Q, A929C, A929P, A929V, V930A, V930E, V930I, V930K, V930M, V930T, S931G, S931P, S931R, D932F, D932R, D932S, D932T, D932V, D933I, D933R, D933S, Q934S, Q934V, I935A, I935C, I935D, I935E, I935L, I935P, I935V, I935W, A936I, A936L, A936Q, A936R, A936Y, K937G, K937I, K937M, K937P, K937Q, K937R, K937V, A938C, A938H, A938N, A938T, A938V, A938W, G939D, G939K, S940C, S940E, S940M, S940R, S940T, S940V, S940W, F941C, F941M, F941W, S942A, S942E, S942K, S942L, S942P, S942T, S942V, V943A, V943G, V943H, V943Q, V943R, A944D, A944G, A944H, A944P, A944R, A944V, G945E, G945P, G945T, T946A, T946E, T946G, T946L, T946P, T946V, T946W, V947G, V947H, V947L, V947M, V947P, V947R, V947T, A948C, A948I, A948R, A948W, G949A, G949F, G949V, Q950D, Q950G, Q950K, Q950M, Q950W, K951D, K951G, K951P, K951Q, K951S, K951W, K951Y, I952H, I952Q, S953F, S953M, S953N, S953R, S953W, V954D, V954L, V954Q, V954S, V954T, R955A, R955C, R955E, R955K, R955Q, R955W, V956A, V956D, V956G, V956H, V956I, V956M, V956Q, V956W, T957D, T957S, T957W, M958D, M958I, M958K, I959A, I959L, I959S, I959V, I959Y, D960G, D960H, D960L, D960P, D960S, D960W, E961D, E961F, E961K, E961P, E961S, E961T, I962A, I962C, I962D, I962G, I962K, I962N, I962Q, I962T, G963C, G963E, G963L, G963P, A964C, A964E, A964H, L965C, L965E, L965G, L965K, L965M, L965P, L965Q, L965S, L965V, L965Y, L966A, L966G, L966H, L966K, L966N, L966P, L966Q, L966S, L966T, L966V, N967C, N967D, N967I, N967L, N967M, N967P, N967S, N967T, N967V, Y968G, Y968L, Y968Q, Y968V, S969C, S969D, S969G, S969H, S969I, S969L, S969M, S969P, S969Q, S969Y, A970I, A970L, S971E, S971F, S971G, S971H, S971V, S971W, P973C, P973D, P973K, P973N, P973Q, P973R, P973V, P973W, P973Y, V974C, V974E, V974G, V974N, V974T, V974Y, G975D, G975F, G975K, G975L, G975Q, G975V, T976D, T976F, T976G, T976K, T976L, T976P, T976S, T976Y, P977A, P977C, P977K, P977R, P977T, P977Y, A978F, A978G, A978M, A978N, A978P, A978R, A978S, A978Y, V979G, V979N, V979R, V979Y, L980A, L980F, L980H, L980I, L980K, L980N, L980Q, L980T, L980Y, P981L, P981M, G982A, G982H, G982M, G982P, G982Q, G982W, R984P, R984S, P985E, P985F, P985H, P985K, P985L, P985W, A986C, A986E, A986F, A986I, A986K, A986L, A986M, A986N, A986S, A986W, V987A, V987C, V987F, V987I, V987K, V987L, V987Q, V987T, L988A, L988C, L988E, L988G, L988H, L988M, L988Q, L988R, L988S, L988V, L988Y, P989A, P989C, P989D, P989G, P989H, P989I, P989M, P989N, P989Q, P989W, D990F, D990P, D990S, D990W, G991C, G991F, G991H, G991K, G991P, G991Y, T992E, T992H, T992M, T992N, T992Y, V993D, V993G, V993N, V993S, T994I, T994S, T994V, S995E, S995L, S995R, S995V, A996Q, A996R, A996V, N997A, N997C, N997E, N997K, N997L, N997S, N997V, N997W, N997Y, F998M, F998W, A999F, A999G, A999L, A999M, A999R, A999S, V1000C, V1000L, V1000M, V1000N, V1000P, V1000W, D1001G, D1001K, D1001L, D1001M, D1001Q, D1001S, D1001T, D1001V, D1001Y, W1002A, W1002D, W1002E, W1002H, W1002N, W1002P, W1002Q, W1002S, T1003F, T1003G, T1003L, T1003N, T1003P, T1003R, T1003S, T1003W, T1003Y, K1004D, K1004E, K1004F, K1004G, K1004H, K1004M, K1004P, K1004R, K1004S, K1004V, P1005I, P1005N, P1005Q, P1005V, P1005Y, A1006C, A1006I, A1006N, A1006P, A1006S, A1006V, A1006W, A1006Y, D1007C, D1007L, D1007P, D1007V, T1008G, V1009G, V1009S, Y1010A, Y1010P, Y1010R, Y1010T, N1011A, N1011S, N1011T, N1011W, T1012E, T1012H, T1012I, T1012Q, T1012Y, A1013D, A1013K, A1013Q, A1013T, A1013V, G1014E, G1014I, G1014L, G1014M, G1014V, G1014W, G1014Y, T1015A, T1015F, T1015G, T1015V, V1016C, V1016D, V1016P, K1017E, K1017G, V1018I, V1018K, V1018L, V1018M, V1018R, V1018S, V1018W, T1021C, T1021E, T1021F, T1021G, T1021K, T1021L, T1021S, T1021V, A1022H, A1022L, A1022S, A1022Y, T1023D, T1023M, T1023Q, T1023R, V1024E, V1024G, V1024H, V1024K, V1024N, V1024R, V1024S, V1024W, G1026E, G1026H, G1026L, G1026R, G1026S, G1026V, G1026Y, K1027C, K1027N, K1027Q, K1027R, K1027V, E1028G, E1028S, E1028T, F1029I, F1029K, F1029L, F1029P, F1029V, F1029W, F1029Y, K1030D, K1030F, K1030H, K1030L, K1030M, K1030W, V1031H, V1031K, V1031Y, A1033G, A1033S, A1033V, T1034G, T1034H, T1034N, T1034W, I1035D, I1035G, I1035Q, R1036G, R1036L, R1036T, R1036Y, V1037C, V1037F, V1037P, V1037Q, Q1038A, Q1038D, Q1038K, R1039S, R1039V, S1040A, S1040M, S1040N, S1040R, S1040W, Q1041P, V1042N, T1043F, T1043G, T1043N, T1043R, I1044A, I1044L, G1045S, S1046I, S1046M, S1047D, V1048C, V1048F, V1048G, V1048I, V1048M, V1048Q, G1050L, G1050S, G1050V, N1051A, N1051E, N1051K, A1052C, A1052K, A1052M, A1052P, A1052R, L1053A, L1053W, R1054C, R1054L, R1054N, L1055R, L1055T, Q1057A, Q1057E, Q1057P, Q1057R, N1058R, N1058S, N1058V, N1058W, I1059W, P1060G, P1060N, P1060Q, P1060S, P1060T, A1061E, A1061G, A1061K, A1061W, D1062A, D1062F, D1062G, D1062I, D1062L, D1062M, D1062P, D1062S, K1063D, K1063M, Q1064C, Q1064M, Q1064R, Q1064T, Q1064V, S1065A, S1065C, S1065E, S1065G, S1065T, S1065W, D1066A, D1066G, D1066M, D1066V, D1066W, T1067G, T1067M, L1068C, L1068E, L1068P, L1068Q, L1068Y, D1069G, D1069K, D1069R, D1069W, A1070P, A1070T, I1071M, I1071R, I1071W, K1072E, K1072G, K1072P, K1072Q, K1072S, D1073F, D1073L, D1073M, D1073P, D1073W, G1074I, G1074L, G1074R, S1075C, S1075G, S1075I, S1075L, S1075V, T1076C, T1076E, T1076H, T1076Q, T1076S, T1077K, T1077L, T1077R, V1078D, V1078E, V1078L, V1078W, D1079G, D1079L, N1081D, N1081E, N1081G, T1082A, T1082C, T1082E, T1082F, T1082G, T1082K, T1082N, T1082S, G1083E, G1083F, G1083L, G1083P, G1083S, G1084C, G1084M, G1084V, G1084W, G1084Y, G1085A, G1085P, G1085R, G1085S, A1086H, A1086K, A1086Q, A1086R, A1086T, N1087A, N1087E, N1087I, N1087R, N1087V, N1087W, P1088D, P1088E, P1088G, P1088R, P1088W, S1089C, S1089E, S1089G, S1089K, S1089Q, S1089R, S1089V, A1090F, A1090G, A1090I, A1090K, W1091A, W1091E, W1091G, W1091H, W1091T, W1091V, W1091Y, T1092A, T1092E, T1092G, T1092K, T1092Q, T1092S, T1092V, N1093A, N1093G, N1093L, N1093P, N1093Q, N1093T, N1093V, W1094D, W1094E, W1094P, W1094R, W1094T, A1095P, A1095R, A1095T, A1095W, Y1096A, Y1096D, Y1096H, Y1096L, Y1096R, S1097D, S1097E, S1097K, S1097L, S1097T, S1097W, K1098D, K1098F, K1098G, K1098Q, K1098S, A1099C, A1099D, A1099F, A1099S, A1099V, A1099W, G1100D, G1100E, G1100H, G1100M, G1100N, G1100T, H1101K, H1101L, H1101Q, H1101R, H1101V, N1102E, N1102F, N1102H, N1102K, N1102L, N1102Q, N1102R, N1102T, T1103A, T1103E, T1103H, T1103S, T1103W, A1104I, A1104K, A1104R, E1105L, E1105S, I1106T, I1106V, T1107C, T1107M, T1107R, T1107S, F1108D, F1108K, F1108L, F1108T, F1108W, E1109A, E1109D, E1109L, E1109W, A1111G, A1111S, E1113D, E1113G, E1113P, E1113V, Q1114E, Q1114I, Q1114L, Q1114R, Q1114S, Q1114T, Q1114V, Q1115A, Q1115K, Q1115L, Q1115P, Q1115T, Q1115W, L1116D, L1116G, L1116H, L1116K, L1116V, L1116W, G1117E, G1117I, G1117M, G1117R, G1117S, G1117T, G1117V, G1117W, Q1118A, Q1118M, Q1118S, Q1118T, Q1118W, I1119D, I1119E, I1119G, I1119N, I1119S, V1120N, V1120S, V1120T, M1121G, M1121K, M1121N, M1121P, M1121S, M1121V, M1121Y, Y1122A, Y1122C, Y1122I, Y1122K, Y1122R, Y1122V, Y1122W, F1123E, F1123H, F1123R, F1123T, F1124E, F1124R, F1124V, F1124W, R1125D, R1125E, R1125F, R1125K, R1125T, R1125V, R1125W, D1126H, D1126K, D1126L, D1126R, S1127F, S1127I, S1127K, S1127M, S1127Q, S1127T, S1127W, N1128A, N1128C, N1128R, N1128S, N1128T, N1128W, A1129E, A1129L, A1129N, A1129Q, A1129R, A1129V, V1130A, V1130G, V1130P, V1130R, V1130S, R1131A, R1131N, R1131Q, R1131S, R1131W, F1132E, F1132K, F1132M, F1132P, F1132Q, F1132T, P1133D, P1133G, P1133L, P1133Q, P1133R, P1133V, D1134E, D1134G, D1134L, A1135E, A1135K, A1135L, A1135M, A1135S, A1135W, A1135Y, G1136A, G1136E, G1136P, G1136Q, G1136T, K1137A, K1137C, K1137G, K1137L, K1137P, K1137Q, K1137R, K1137S, K1137T, K1137V, T1138R, T1138Y, K1139A, K1139L, K1139R, K1139T, I1140A, I1140C, I1140G, I1140L, I1140M, I1140P, I1140R, I1140T, Q1141A, Q1141C, Q1141G, Q1141K, Q1141N, Q1141P, Q1141T, Q1141W, I1142E, I1142R, I1142S, I1142W, I1142Y, S1143G, A1144C, A1144D, A1144E, A1144N, A1144P, A1144S, A1144T, A1144V, A1144W, D1145E, D1145R, D1145T, G1146A, G1146C, G1146D, G1146K, G1146L, G1146R, G1146V, K1147A, K1147G, K1147T, K1147V, N1148H, N1148I, N1148K, N1148P, N1148Q, N1148R, N1148S, N1148T, N1148W, W1149C, W1149G, W1149I, W1149K, W1149N, W1149Q, W1149S, W1149T, W1149V, W1149Y, T1150G, T1150K, T1150P, D1151C, D1151G, D1151R, D1151T, D1151W, L1152A, L1152C, L1152E, L1152Q, L1152W, A1153E, A1153G, A1153K, A1153L, A1154C, A1154D, A1154E, A1154G, A1154R, A1154S, T1155E, T1155L, T1155Q, T1155R, T1157V, T1157W, I1158R, I1158S, I1158W, A1159C, A1159E, A1159I, A1159P, A1159R, A1159V, A1160K, A1160L, A1160Q, A1160S, Q1161A, Q1161P, Q1161S, E1162A, E1162C, E1162D, E1162F, E11621, E1162N, E1162Q, E1162T, E1162W, E1162Y, E1165D, E1165H, E1165L, E1165M, E1165R, E1165S, E1165W, R1166D, R1166K, R1166Q, V1167A, V1167C, V1167L, V1167P, V1167R, K1168L, K1168Q, K1168R, K1168W, P1169M, P1169R, P1169S, Y1170E, Y1170K, Y1170M, Y1170Q, Y1170R, Y1170V, T1171A, T1171G, T1171M, T1171Q, T1171R, T1171S, Y1172D, Y1172E, Y1172H, Y1172I, Y1172K, Y1172L, Y1172S, Y1172V, D1173A, D1173E, D1173F, D1173G, D1173K, D1173L, D1173P, D1173R, D1173T, D1173W, F1174D, F1174P, F1174Q, F1174R, F1174S, F1174T, F1174V, F1174W, A1175G, A1175I, A1175N, A1175Q, A1175S, A1175V, A1175Y, V1177N, V1177P, V1177S, V1177T, G1178M, G1178Q, G1178S, G1178T, A1179L, A1179P, A1179Q, A1179W, T1180A, T1180G, T1180I, T1180L, T1180M, T1180Q, T1180S, T1180Y, F1181E, F1181L, F1181V, V1182M, K1183A, K1183E, K1183T, K1183V, V1184C, V1184E, V1184K, V1184L, V1184P, V1184Q, V1184Y, T1185Q, T1185V, V1186S, N1188V, N1188W, A1189C, A1189K, A1189T, A1189V, D1190R, D1190S, D1190T, D1190Y, T1191E, T1191L, T1192H, T1192P, T1193G, P1194A, P1194E, P1194G, P1194W, S1195G, V1197A, V1198E, C1199D, C1199T, A1200G, A1200V, A1200W, L1202A, L1202C, T1203E, T1203K, E1204G, E1204S, I1205V, K1208R, K1208V, K1208W, T1209A, T1209C, T1209E, T1209K, T1209N, T1209Q, T1209R, T1209W, A1210D, A1210E, A1210G, A1210K, A1210L, A1210Q, A1210R, A1210T, A1210W, T1211C, T1211D, T1211E, T1211G, T1211H, T1211K, T1211P, T1211Q, T1211R, T1211S, T1211V, K1213A, K1213D, K1213S, K1213T, K1213W, F1214A, F1214E, F1214K, F1214L, F1214P, F1214R, F1214S, F1214V, V1215D, V1215E, V1215K, V1215L, V1215Q, V1215S, V1215W, T1216A, T1216L, T1216P, T1216Q, T1216R, N1217A, N1217D, N1217E, N1217F, N1217P, N1217R, N1217S, N1217T, T1218A, T1218C, T1218E, T1218G, T1218Q, T1218S, T1218V, T1218W, S1219A, S1219E, S1219F, S1219I, S1219K, S1219R, S1219V, A1220C, A1220G, A1220L, A1220P, A1220R, A1220V, A1221D, A1221G, A1221K, A1221L, A1221R, A1221V, A1221W, L1222A, L1222C, L1222E, L1222F, L1222Q, L1222R, L1222V, L1222W, S1223C, S1223F, S1223G, S1223K, S1223L, S1223V, S1224A, S1224D, S1224G, S1224L, S1224M, S1224P, S1224R, S1224W, L1225C, L1225D, L1225E, L1225F, L1225G, L1225K, L1225P, L1225T, L1225V, L1225W, T1226A, T1226G, T1226M, T1226P, T1226R, T1226S, T1226V, T1226Y, V1227A, V1227C, V1227D, V1227E, V1227G, V1227L, V1227P, V1227Q, V1227S, N1228A, N1228D, N1228F, N1228K, N1228L, N1228T, G1229A, G1229C, G1229E, G1229Q, G1229S, G1229V, T1230F, T1230H, T1230I, T1230K, T1230L, T1230P, T1230R, T1230S, T1230W, K1231F, K1231G, K1231L, K1231M, K1231P, K1231S, K1231W, V1232E, V1232K, V1232Q, V1232R, V1232S, V1232T, V1232W, S1233P, S1233W, D1234G, D1234K, D1234R, D1234V, S1235D, S1235E, S1235G, S1235L, S1235P, S1235R, S1235W, S1235Y, V1236A, V1236C, V1236G, V1236I, V1236P, V1236Q, V1236R, L1237D, L1237E, L1237R, L1237V, L1237W, A1238D, A1238E, A1238K, A1238L, A1238N, A1238P, A1238R, A1238S, A1238T, A1239D, A1239P, A1239R, G1240D, G1240L, G1240N, G1240Q, G1240S, G1240T, G1240W, S1241D, S1241G, S1241I, S1241L, S1241M, S1241P, Y1242C, Y1242E, Y1242K, Y1242R, Y1242S, Y1242W, N1243C, N1243L, N1243M, N1243P, N1243Q, N1243S, N1243T, N1243V, N1243W, T1244A, T1244D, T1244E, T1244G, T1244L, T1244Q, T1244S, T1244V, T1244W, A1246F, A1246M, A1246N, A1246P, A1246Q, A1246R, A1246S, A1246T, I1247A, I1247G, I1247M, I1247Q, I1247R, I1247S, I1247T, I1247V, I1247W, I1248A, I1248G, I1248K, I1248L, I1248R, I1248S, I1248Y, A1249E, A1249G, A1249H, A1249I, A1249R, A1249T, A1249V, D1250I, D1250K, D1250S, D1250T, D1250W, D1250Y, V1251I, V1251T, V1251W, K1252D, K1252G, K1252V, K1252W, A1253P, A1253V, E1254F, E1254G, E1254H, E1254L, E1254R, E1254V, G1255H, G1255M, G1255S, G1255V, E1256G, E1256M, E1256N, E1256R, E1256V, E1256W, G1257F, G1257K, G1257L, G1257Q, G1257R, G1257W, N1258C, N1258G, N1258H, N1258K, N1258S, A1259K, A1259L, A1259W, V1261I, V1261L, V1261P, V1261Q, V1261R, V1261T, T1262A, T1262F, T1262M, T1262Q, T1262R, V1263E, V1263G, V1263Q, V1263R, V1263T, V1263W, L1264A, L1264E, L1264H, L1264R, L1264S, L1264Y, P1265C, P1265K, P1265L, P1265R, P1265S, P1265V, P1265W, A1266F, A1266L, A1266P, A1266S, A1266V, H1267A, H1267E, H1267F, N1269A, N1269E, N1269K, N1269R, N1269S, N1269T, N1269W, V1270D, V1270E, V1270G, V1270I, V1270L, V1270T, V1270W, I1271A, I1271H, I1271Q, R1272E, R1272F, R1272M, R1272P, R1272V, V1273R, I1274F, I1274M, I1274R, T1275A, T1275L, T1275W, E1276R, E1276W, S1277L, S1277T, S1277W, E1278Q, E1278R, D1279G, D1279I, D1279R, D1279T, D1279V, D1279W, H1280C, H1280E, H1280G, H1280V, H1280W, V1281F, V1281I, V1281S, V1281W, T1282D, T1282L, T1282V, R1283A, R1283D, R1283E, R1283P, R1283W, K1284G, T1285A, T1285E, T1285F, T1285G, T1285M, T1285R, T1285Y, F1286A, F1286E, F1286P, F1286R, F1286S, F1286T, T1287C, T1287K, T1287L, T1287M, T1287Q, T1287R, T1287S, T1287W, I1288A, I1288D, I1288F, I1288G, I1288K, N1289A, N1289Q, N1289T, L1290A, L1290G, L1290R, L1290V, L1290W, G1291K, G1291P, G1291V, G1291W, G1291Y, T1292G, T1292L, T1292Y, E1293G, E1293K, E1293L, E1293S, E1293V, Q1294E, Q1294L, Q1294P, Q1294W, E1295K, F1296A, F1296G, F1296I, P1297F, A1298Y, D1301I, E1302G, E1302R, E1302S, R1303S, D1304A or D1304V.

In preferred embodiments, the lactase variants of the present invention comprise one or more of the substitutions listed in any one of embodiments 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144 or 146 of the “PREFERRED EMBODIMENTS” section right before the “EXAMPLES” section of the present application.

In a particularly preferred embodiment, the lactase variants of the invention have an amino acid sequence which is at least 99.4% identical to SEQ ID NO: 1, have a galactose inhibition improvement factor (GI-IF) of at least 1.4 or at most 0.6 compared to the lactase of SEQ ID NO: 1 and comprise one or more of the following substitutions, wherein each position corresponds to a position in SEQ ID NO: 1: E2V, D3I, D3S, D3V, S7P, M13R, S15C, S15I, S15K, S15Y, T16C, V20P, Y21C, Y21R, Y21T, S23L, S23M, A24F, A24T, D26C, S27A, K28C, Q29L, Q29M, Q29R, R31E, R31V, T32S, D34S, F35E, K40F, K40I, M42E, L43T, D45P, V47K, V47R, A49D, A49H, A49R, A49T, D51I, D51K, D51P, A53S, D55H, D55M, S57C, S57E, S57G, A58T, W59P, V62N, L64E, H66Y, K73D, S75G, S75L, S75R, Q76G, S77E, S77F, S77H, S77K, N78C, E79H, E79S, E79T, E81A, E81Q, L85F, L85S, L85V, L85W, P86Q, G87D, G88F, G88Q, G90T, W91Y, Y92H, Y92M, Y92S, Y92V, R93A, R93D, R93F, R93H, R93L, R93N, R93T, R93V, R93Y, K94S, S95C, S95G, S95I, F96A, F96C, F96I, F96S, F96V, T97F, I98H, R100T, D101A, D101P, L102P, I109M, N110T, F111A, F111L, D112T, V114R, Y115E, V120A, W121R, W121V, G124E, H130A, H130T, Y132E, P136R, F137L, K146A, G149M, E150L, I153A, V157P, E158G, E158H, N159T, L161K, L161S, L161W, P162N, P162T, P162W, R165H, W166S, Y167A, S170Q, I172P, R174E, V176E, V176K, T177K, L178I, L178Q, T179C, V180G, H185G, H185L, N188V, N189E, G190C, S197C, D217L, D217M, T218G, A220G, A220V, A222R, N223S, Q228R, K233P, K236M, T237D, A239G, A239T, G242Y, A247P, A247R, S248E, K249G, K249Y, I251Y, A252F, A252W, A252Y, G254I, G254R, A255K, S256Y, S261R, T262D, T264M, P268R, S272W, L278V, Y279W, L286F, V291D, V291P, L292V, D293C, D296F, E298I, E298R, F301C, W303A, T304P, G305N, G305P, G305W, T309D, F312H, K320V, K320W, S325G, M326E, M326G, M326T, M326Y, H328C, H328M, H328T, A337G, A337L, R340A, A341M, I342K, E343N, Q345G, Q345K, Q345S, I348D, Q350G, T359E, T360V, N362S, N362T, P363I, P363L, P363M, A364I, A364M, A364P, A365C, L368E, L368Q, L368S, I369G, I369K, I369W, D370L, N373G, N373L, G376A, G376S, V377M, V379A, V380P, E381A, E381T, M386G, M386N, M386S, W387L, N388L, N388R, R389A, R389E, R389Q, R389S, R389T, S390C, S390D, S390G, S390P, S390Q, S390T, K391E, N392D, G393R, G393V, N394L, T395C, T395H, T395N, T395S, T395W, E396K, E396L, E396V, E396W, Y398N, G399S, K400A, K400C, K400N, K400P, K400S, K400V, W401F, W401K, F402W, F402Y, G403A, G403K, G403V, Q404F, Q404H, Q404L, Q404M, Q404P, Q404V, A405C, A405E, A405H, A405R, I406D, I406N, G408W, D409N, A411E, L413P, G414A, G414R, D416I, D416Y, K417F, D418P, D418R, E419M, E419R, T420K, T420R, K423D, F424C, F424L, L426M, T427D, T427Q, T427S, T429D, N431M, D433P, D433W, R434N, R434V, N435L, A436L, V439C, V439G, V439K, V439T, W442E, W442G, W442R, S443C, S443Y, L444V, G445A, G445C, G445V, M448C, M448L, M448S, M449D, M449T, M449W, M449Y, E450F, E450S, E450T, E450V, E450W, G451C, G451F, G451L, G451P, G451V, G451W, I452K, G454L, S455P, S455R, V456D, V456E, V456F, V456K, S457T, S457V, G458A, G458P, F459R, F459S, K465Y, L466P, L466V, A468D, A472G, A472Y, S475E, R477L, M479G, M479R, K485E, K487F, K487N, A488N, N491W, S493M, S493Q, T495W, D498C, N499R, N499T, N499Y, L500A, G504H, G505D, G505S, V506C, V506E, V506R, V507L, V507R, G508C, G508E, T509K, T509M, N510A, N510F, N510I, Y511A, S512F, S512T, S512Y, D513L, D513P, D513R, G514F, A515E, N516C, Y517G, D518Y, K519C, K519I, I520G, I520H, I520S, R521N, R521V, T522N, H524R, H524V, P525T, S526G, W527N, W527R, W527S, A528E, G531T, A537P, S540E, R546L, R546P, R546S, T547A, T547S, T548F, T548K, T548P, G549P, G549W, G550S, A551Q, S553N, S554N, D555S, K556W, L558P, T559Y, S564C, S564F, A565E, A565I, A565R, G567Q, G567V, A570K, A570M, A572S, S574W, W576Y, Y577L, V579G, R582L, D583W, V585I, A586K, T588D, T592C, T592L, T592S, F594L, D595Q, P600A, P600E, P600G, P600S, G607Q, S608V, G609N, G609R, A610M, V611K, W614A, N619L, S620G, V625M, V625Y, F630G, T634V, Y635R, Q639R, D644C, H650R, W655R, N656V, A661E, P669T, V670C, Y673S, A676P, K680I, T684V, G687P, S688L, R692H, I694L, I694W, G695C, G695L, G695W, K697R, S698E, T700C, K701A, K701G, K701M, A705W, T709M, Y710T, V712M, E714K, A716R, D717G, D717S, K718A, D719T, D719V, T721N, T721S, T721W, H723G, M726D, L728S, N731S, W734G, W734V, A735Q, A735S, I739K, S740D, A741I, D745F, L750S, P752A, P752C, P752Y, G754P, T756N, T756S, E757A, A760N, A769F, A769I, A769N, A769V, A769Y, K770H, K772A, K772C, K772P, A773C, A773H, A773V, D774A, A775L, A775W, D776I, D776L, D776S, R777D, R777E, R777G, R777H, R777P, R777S, T781A, T781G, T781P, T781Y, A782Q, D783C, D783E, D783R, G784L, G784S, K785S, K785W, D786V, L787P, L787T, Y789V, V792G, D793H, V794T, T795P, N798I, G799K, G799L, G799M, G799Q, H800A, H800L, P803S, A806Q, R808C, T810P, V813T, K814H, G815A, A816C, A816F, K818F, K818L, K818W, G821E, G821I, V822D, V822E, S826L, H830R, D831I, D831P, D831V, S832R, A835D, A835H, A835W, D836E, D836H, D836S, D836Y, R838G, K839D, A840G, K844G, Q850C, Q850G, E854C, I858D, I858Q, I858Y, K863F, K863N, D865A, D865G, L867W, S870R, V872C, I874G, S883L, T887F, T887G, V888A, Y892P, Y892R, Y893E, T901V, T901Y, G902R, G902S, G902W, N903D, K904M, K904V, P905R, P905Y, I906W, P908I, D910W, E912T, R914A, R914F, R914K, R914Y, Y915A, Y915C, Y915M, Y915V, S916G, D917C, D917F, D917S, G918H, T919Q, D921V, R922A, Q923V, W927G, Q934S, I935P, A936Q, K937I, K937P, A938C, S940T, S942K, S942V, V943A, V943H, V943R, A944P, T946P, T946V, V947G, A948C, Q950D, V956Q, E961D, E961F, E961S, I962C, I962N, L965C, L965G, L965M, L965P, L965Y, L966A, Y968G, Y968V, S969C, S969I, S969M, P973C, T976K, T976P, P977T, A978M, V979R, L980H, L980N, P981M, P985L, P985W, A986I, V987K, D990W, S995L, N997K, D1001L, W1002A, W1002D, W1002H, W1002N, W1002P, T1003Y, K1004H, A1006S, D1007P, V1018K, A1022H, V1024G, G1026R, K1030W, I1035D, V1048M, V1048Q, G1050S, N1051E, A1052K, L1053A, Q1057E, Q1057R, D1066W, L1068C, I1071W, K1072G, K1072Q, T1082F, G1083E, G1084V, G1085S, P1088E, S1089Q, W1091A, W1091V, T1092Q, N1093P, N1093Q, W1094D, W1094E, S1097W, A1099V, N1102E, N1102H, A1104R, T1107S, E1109D, E1109L, E1109W, A1111G, E1113P, Q1114S, Q1114V, Q1115A, Q1115K, L1116K, L1116W, G1117I, G1117S, G1117T, G1117V, G1117W, I1119N, R1125V, R1125W, S1127Q, N1128A, A1129E, V1130A, V1130G, V1130R, P1133D, P1133R, G1136E, K1137R, K1137S, Q1141P, A1144P, A1144S, D1145E, D1145R, D1145T, T1150K, D1151R, D1151W, V1167R, A1175N, G1178M, A1179W, T1180A, T1180G, T1180I, T1180M, T1180Q, T1180Y, V1184E, V1184L, V1184P, V1186S, N1188W, C1199T, K1208W, T1209E, T1209K, A1210D, A1210G, A1210R, A1210T, A1210W, T1211G, T1211P, T1211R, F1214A, F1214K, F1214V, V1215D, T1216L, T1216Q, N1217A, T1218W, S1219A, S1219F, L1222F, L1222V, S1223V, S1224P, S1224W, L1225E, L1225K, T1226P, N1228F, G1229V, T1230K, T1230W, K1231F, S1233P, A1238E, A1238R, N1243T, T1244Q, A1246M, D1250I, D1250S, D1250W, A1259K, P1265W, N1269A, N1269K, I1274F, I1274R, T1275W, S1277W, E1278Q, D1279R, D1279W, H1280E, V1281I, V1281W, K1284G, F1286E, T1287C, T1287W, I1288A, I1288D, I1288G, I1288K, E1302R, E1302S or D1304V. More preferably, the lactase variants of this embodiment, having an amino acid sequence which is at least 99.4% identical to SEQ ID NO: 1 and having a galactose inhibition improvement factor (GI-IF) of at least 1.4 or at most 0.6 compared to the lactase of SEQ ID NO: 1, comprise one or more of the substitutions listed in any one of embodiments 108, 116, 124, 132 or 140 of the “PREFERRED EMBODIMENTS” section right before the “EXAMPLES” section of the present application.

In another particularly preferred embodiment, the lactase variants of the invention have an amino acid sequence which is at least 99.4% identical to SEQ ID NO: 1, have a galactose inhibition improvement factor (GI-IF) of at least 1.3 compared to the lactase of SEQ ID NO: 1 and comprise one or more of the following substitutions, wherein each position corresponds to a position in SEQ ID NO: 1: V1H, V1L, E2D, E2V, D3I, D3V, S7N, S7P, M13C, M13R, S14V, S15C, S15I, S15K, S15T, S15V, T16C, P17I, V20C, V20M, V20P, Y21C, Y21P, Y21R, Y21T, S22W, A24F, A24L, A24T, A24W, V25Q, D26C, S27A, S27C, S27H, K28C, Q29L, Q29M, Q29R, R31E, R31V, T32S, F35C, W39G, K40C, K40F, K40I, F41A, M42T, L43S, D45P, V47K, V47R, A49D, A49H, A49T, D51I, D51K, D55C, D55H, D55M, D55P, S57C, S57E, S57G, A58Q, A58T, W59I, W59K, W59P, Q60R, Q60S, V62N, V62T, V62W, L64E, H66R, K73D, S75G, S75L, S75R, S75V, Q76G, Q76I, S77D, S77E, S77G, S77H, S77K, S77R, N78C, E79H, E79S, E79T, A83T, L85A, L85F, L85N, L85S, L85V, L85W, P86Q, G87D, G88F, G90T, W91P, W91Y, Y92H, Y92I, Y92M, Y92S, Y92T, Y92V, R93A, R93D, R93F, R93H, R93I, R93L, R93N, R93T, R93V, R93Y, K94A, K94S, K94T, S95C, S95D, S95G, S95I, F96A, F96C, F96I, F96K, F96M, F96P, F96V, T97F, I98C, I98H, R100T, D101A, D101P, L102P, R106W, I107S, A108E, I109M, N110A, N110S, N110T, N110W, F111A, F111L, V114R, Y115E, M116C, A118P, V120A, V120K, W121R, W121V, H130A, H130T, Y132E, F137L, T142S, G149M, E150L, V154K, V154L, V157P, V157S, E158H, N159T, L161K, L161M, L161S, L161W, P162N, P162T, P162W, S164A, R165H, W166S, Y167A, S170Q, I172K, I172P, Y173H, R174E, V176E, V176K, T177K, L178I, L178Q, T179C, T179I, T179N, V180D, V180G, T181A, V184R, H185G, H185L, G187D, N188V, G190F, K194I, S197C, A199P, Q201E, N202Q, N202S, N202W, D217L, D217M, T218G, A220G, A222R, N223S, Q228R, K236M, T237D, G242K, I251Y, A252W, A252Y, S261H, A266D, S267V, P268R, S272T, S272W, V281Q, V291P, L292V, D293C, D293W, Y295F, D296F, D296K, E298I, E298R, F301C, W303A, W303C, W303D, T304D, T304K, T304P, G305N, G305P, T309C, T309D, T309E, S310H, S310L, S310N, F312H, L314A, L314V, N315S, K318N, K320V, K320W, L321M, K322I, V324T, S325G, M326T, H328C, H328F, H328G, H328M, H328T, G331V, A335G, A335L, A337G, A337L, R340A, R340L, A341M, I342G, E343N, E343Y, R344G, Q345S, V346A, E347A, I348D, I348M, Q350G, M352T, G353K, I357T, T359E, T359L, T360V, N362S, N362T, P363A, P363I, P363L, P363M, A364I, A364M, A364P, A365C, A365I, A365P, A365V, K366L, K366M, K366S, L368A, L368Q, L368S, I369E, I369G, I369K, I369V, I369W, D370L, V371F, V371Q, C372P, N373G, N373L, G376A, G376S, V377M, V377T, V379A, V379N, V380P, E381A, E381G, E381Q, E381T, V383A, M386G, M386N, M386S, W387H, N388L, R389A, R389E, R389Q, R389S, R389T, S390C, S390D, S390P, S390T, G393E, G393R, G393V, T395C, E396K, E396L, E396V, E396W, Y398M, G399S, K400A, K400C, K400N, K400V, W401F, W401K, F402W, F402Y, G403A, G403K, G403Q, G403V, Q404F, Q404L, Q404M, Q404P, Q404V, A405C, A405E, A405K, A405R, I406D, I406N, A407C, A407G, A407Q, N410Y, A411E, G414M, G414N, K417F, K417R, A422P, T427D, T427F, T427Q, T427S, T429D, N431M, D433G, D433I, D433P, D433Q, D433W, R434N, R434V, N435E, N435L, A436L, V439C, V439K, W442E, W442G, S443C, S443G, S443Q, L444E, L444V, N446D, M448C, M448L, M449D, M449T, M449W, M449Y, E450F, E450S, E450T, E450V, E450W, G451C, G451F, G451L, G451P, G451V, G451W, I452K, I452S, I452V, G454L, S455P, S455R, V456D, V456E, V456F, V456K, S457H, S457T, S457V, G458A, G458P, G458S, F459R, A461M, T462E, T462L, K465V, K465Y, L466P, A468D, K471F, A472G, A472Y, S475E, R477L, M479G, K485E, K485R, K487F, K487S, K487W, S493G, S493L, S493Q, N494R, G497D, N499T, G505D, G505R, G505S, V506C, V506E, V506R, V507R, G508C, G508E, T509A, T509K, T509M, N510A, N510F, N510I, Y511A, S512F, S512Q, S512T, S512V, S512Y, D513P, D513R, A515E, N516C, N516E, N516Q, N516V, Y517G, D518Y, K519C, K519I, I520C, I520G, I520H, I520M, I520S, I520T, R521V, H524R, H524V, S526G, W527A, W527H, W527N, W527R, W527S, A528E, A537M, A537P, S540E, G549D, G549P, G549W, L558E, L558P, T559Y, S564C, S564F, S564W, A565D, A565E, A565I, A565L, A565R, A565T, G567Q, G567V, A570G, A575D, R582L, D583W, W591F, T592L, G598N, P600A, P600E, P600G, P600S, G607Q, W614A, N619I, N619L, V625M, V625Y, T627K, T627Q, A628D, F630C, F630Y, P631D, T634V, Q639R, W655R, E657R, A661E, A661K, A661Q, N667L, N667R, P669T, Y673S, T674M, A676P, V679S, K680I, G687P, S688K, S688L, T689W, E690D, R692G, R692H, I694L, I694W, G695C, G695L, G695W, K697R, S698E, S698M, K701A, K701G, K701M, K701S, A705W, Y708F, Y710T, V712M, V712Q, E714K, A716R, D717G, D717S, K718A, K718T, D719E, D719T, D719V, T721H, T721L, T721N, T721S, T721V, T721W, H723G, M726D, Y727L, L728S, N731E, V732R, W734G, A735Q, A735S, T738S, I739K, S740D, A741I, Y744R, D745N, L750S, P752C, P752Y, T756N, E757A, A769F, A769I, A769N, A769V, A769Y, K770H, L771A, K772A, K772C, K772P, A773C, A773M, A773R, D774A, D776V, R777H, R777S, K778N, T781A, T781F, T781G, T781P, T781Y, A782Q, D783A, K785S, K785W, K785Y, D786V, Y789V, I790R, N798I, G799K, G799L, V802Y, P803S, A806I, A806Q, R808F, T810P, T810Q, V813W, K818F, K818L, K818W, K818Y, L819F, G821I, V822D, V822E, S826G, D829S, D831P, S832R, A835D, A835H, A835K, D836C, D836E, D836S, D836V, D836Y, R838N, K839A, K839D, A840G, A840P, A840V, E854C, I858D, T861W, D865A, D865G, G866H, L867W, S870E, V872C, K873G, K873Y, I874G, S883L, K886V, K886W, T887G, V888A, Y892P, Y893E, Y897V, K900E, T901Q, T901R, T901Y, G902F, G902R, G902S, G902W, N903D, K904M, K904N, K904S, K904V, P905C, P905R, P905Y, I906W, L907F, P908I, P908L, D910W, E912T, R914A, R914F, R914I, R914K, R914V, R914Y, Y915A, Y915C, Y915G, Y915M, Y915Q, Y915V, S916G, D917C, D917F, D917S, G918E, G918H, G918T, T919D, T919Q, D921V, R922A, Q923V, T926R, W927P, A929P, D933R, Q934S, I935A, I935C, I935E, I935P, I935W, A936I, A936Q, K937G, K937I, K937P, K937Q, A938C, G939D, S940C, F941C, S942K, S942V, V943A, T946P, V947G, V947R, V947T, A948W, K951S, S953M, R955C, R955W, V956D, V956Q, E961D, E961F, E961S, I962C, I962K, I962N, L965C, L965G, L965K, L965M, L965P, L965Y, L966A, N967M, Y968G, Y968Q, Y968V, S969C, S969I, S969L, S969M, S969Q, A970I, S971V, P973C, P973W, V974T, T976K, T976P, P977C, P977T, A978F, A978M, L980H, L980N, P981M, P985L, P985W, A986I, A986L, V987A, V987F, V987K, D990W, G991Y, S995L, N997K, D1001L, W1002A, W1002D, W1002E, W1002H, W1002N, W1002P, W1002Q, T1003Y, P1005Y, A1006C, A1006S, D1007P, N1011T, T1012Q, G1014I, V1018K, A1022H, V1024G, V1024H, G1026V, K1030D, K1030W, I1035D, Q1041P, V1042N, V1048C, V1048G, V1048M, V1048Q, N1051E, N1051K, A1052K, L1053A, Q1057E, A1061G, D1062M, Q1064M, S1065A, D1066V, T1067M, L1068P, K1072S, G1074L, T1082F, G1083E, G1083F, G1083L, G1084V, G1085S, P1088E, P1088R, S1089G, S1089Q, W1091Y, N1093A, N1093P, N1093Q, W1094D, W1094E, K1098D, A1099V, H1101V, N1102H, T1103E, E1109A, E1109D, E1109L, E1109W, A1111G, E1113P, E1113V, Q1114E, Q1114R, Q1114S, Q1114T, Q1114V, Q1115A, Q1115K, L1116K, L1116W, G1117I, G1117R, G1117S, G1117T, G1117V, G1117W, I1119N, M1121G, R1125V, R1125W, S1127I, S1127Q, N1128A, A1129E, V1130A, V1130R, P1133D, P1133R, A1135L, A1135W, G1136P, K1137R, K1137S, I1140P, Q1141K, I1142Y, A1144N, A1144P, A1144S, A1144V, A1144W, D1145E, D1145R, D1145T, N1148P, W1149C, W1149V, T1150K, D1151W, L1152W, A1153G, E1162A, V1167R, G1178M, G1178T, A1179L, A1179W, T1180A, T1180G, T1180I, T1180M, T1180Q, T1180Y, V1184E, V1184L, V1184P, V1184Q, V1184Y, V1186S, N1188W, D1190T, T1191L, V1197A, E1204G, K1208W, T1209E, T1209K, A1210D, A1210G, A1210K, A1210L, A1210R, A1210T, A1210W, T1211G, T1211K, T1211P, T1211R, T1211S, F1214A, F1214K, F1214S, F1214V, V1215D, V1215L, T1216L, T1216Q, N1217A, N1217S, N1217T, T1218C, T1218E, T1218S, T1218W, L1222A, L1222E, L1222F, L1222V, S1224P, S1224W, L1225E, L1225K, T1226P, G1229V, T1230K, T1230W, V1232R, S1235W, V1236A, V1236G, V1236P, A1238E, A1238R, G1255H, A1259K, T1262R, V1263T, P1265W, I1274F, I1274R, T1275W, S1277L, S1277T, S1277W, E1278Q, D1279R, D1279W, H1280E, V1281I, V1281W, K1284G, F1286E, F1286P, T1287C, T1287S, T1287W, I1288A, I1288D, I1288G, I1288K, E1295K, F1296A, E1302R, E1302S or D1304V. More preferably, the lactase variants of this embodiment, having an amino acid sequence which is at least 99.4% identical to SEQ ID NO: 1 and having a galactose inhibition improvement factor (GI-IF) of at least 1.3 compared to the lactase of SEQ ID NO: 1, comprise one or more of the substitutions listed in any one of embodiments 104, 112, 120, 128, 136 or 144 of the “PREFERRED EMBODIMENTS” section right before the “EXAMPLES” section of the present application.

In one aspect, a variant of the present invention comprises a substitution at position V1 to A, C, D, E, F, G, H, I, K, L, M, N, P, Q, R, S, T, W or Y, preferably to D, F, H, K, L or R, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ

In another aspect, the variant comprises a substitution at position E2 to A, C, D, F, G, H, I, K, L, M, N, P, Q, R, S, T, V, W or Y, preferably to D, G, Q or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position D3 to A, C, E, F, G, H, I, K, L, M, N, P, Q, R, S, T, V, W or Y, preferably to A, H, I, N, S, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A4 to C, D, E, F, G, H, I, K, L, M, N, P, Q, R, S, T, V, W or Y, preferably to C, G, H, I, L, M or P, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T5 to A, C, D, E, F, G, H, I, K, L, M, N, P, Q, R, S, V, W or Y, preferably to A, D, F, K, R, S or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position R6 to A, C, D, E, F, G, H, I, K, L, M, N, P, Q, S, T, V, W or Y, preferably to A, D, F, G, H, L, M, P, S or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1. In another aspect, the variant comprises a substitution at position S7 to A, C, D, E, F, G, H, I, K, L, M, N, P, Q, R, T, V, W or Y, preferably to A, D, I, L, N, P, T or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1. In another aspect, the variant comprises a substitution at position S9 to A, C, D, E, F, G, H, I, K, L, M, N, P, Q, R, T, V, W or Y, preferably to G, H, P, R or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T10 to A, C, D, E, F, G, H, I, K, L, M, N, P, Q, R, S, V, W or Y, preferably to G, K, L, P, R, S or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T11 to A, C, D, E, F, G, H, I, K, L, M, N, P, Q, R, S, V, W or Y, preferably to L or P, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position Q12 to A, C, D, E, F, G, H, I, K, L, M, N, P, R, S, T, V, W or Y, preferably to L, R, V or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position M13 to A, C, D, E, F, G, H, I, K, L, N, P, Q, R, S, T, V, W or Y, preferably to C, D, E, F, H, K, R or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position S14 to A, C, D, E, F, G, H, I, K, L, M, N, P, Q, R, T, V, W or Y, preferably to A, G, H, L, T, V or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position S15 to A, C, D, E, F, G, H, I, K, L, M, N, P, Q, R, T, V, W or Y, preferably to C, F, I, K, L, P, R, T, V, W or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T16 to A, C, D, E, F, G, H, I, K, L, M, N, P, Q, R, S, V, W or Y, preferably to A, C, I, N or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position P17 to A, C, D, E, F, G, H, I, K, L, M, N, Q, R, S, T, V, W or Y, preferably to A, C, D, E, I, L, N, R, S or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1. In another aspect, the variant comprises a substitution at position V19 to A, C, D, E, F, G, H, I, K, L, M, N, P, Q, R, S, T, W or Y, preferably to A, F, G, I, K, L, N, S or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1. In another aspect, the variant comprises a substitution at position V20 to A, C, D, E, F, G, H, I, K, L, M, N, P, Q, R, S, T, W or Y, preferably to C, F, G, I, K, L, M, N, P, Q, R, T or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ

In another aspect, the variant comprises a substitution at position Y21 to A, C, D, E, F, G, H, I, K, L, M, N, P, Q, R, S, T, V or W, preferably to A, C, D, F, G, H, M, P, R or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position S22 to A, C, D, E, F, G, H, I, K, L, M, N, P, Q, R, T, V, W or Y, preferably to A, E, F, G, L, M, N, R, T or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position S23 to A, C, D, E, F, G, H, I, K, L, M, N, P, Q, R, T, V, W or Y, preferably to A, C, D, L, M or R, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A24 to C, D, E, F, G, H, I, K, L, M, N, P, Q, R, S, T, V, W or Y, preferably to F, G, L, R, T or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V25 to A, C, D, E, F, G, H, I, K, L, M, N, P, Q, R, S, T, W or Y, preferably to D, E, F, G, H, K, L, M, Q, R, S, T or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position D26 to A, C, E, F, G, H, I, K, L, M, N, P, Q, R, S, T, V, W or Y, preferably to C, I, L, M, T or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1. In another aspect, the variant comprises a substitution at position S27 to A, C, D, E, F, G, H, I, K, L, M, N, P, Q, R, T, V, W or Y, preferably to A, C, F, G, H, P or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1. In another aspect, the variant comprises a substitution at position K28 to A, C, D, E, F, G, H, I, L, M, N, P, Q, R, S, T, V, W or Y, preferably to C, G, I, L, R, S, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1. In another aspect, the variant comprises a substitution at position Q29 to A, C, D, E, F, G, H, I, K, L, M, N, P, R, S, T, V, W or Y, preferably to D, F, G, L, M, R, S, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position N30 to A, C, D, E, F, G, H, I, K, L, M, P, Q, R, S, T, V, W or Y, preferably to A, G, H, M, P, V, W or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position R31 to A, C, D, E, F, G, H, I, K, L, M, N, P, Q, S, T, V, W or Y, preferably to E, G, I, M or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T32 to A, C, D, E, F, G, H, I, K, L, M, N, P, Q, R, S, V, W or Y, preferably to M, Q, R or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position S33 to A, C, D, E, F, G, H, I, K, L, M, N, P, Q, R, T, V, W or Y, preferably to C, E, H, K, N, Q, R or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1. In another aspect, the variant comprises a substitution at position D34 to A, C, E, F, G, H, I, K, L, M, N, P, Q, R, S, T, V, W or Y, preferably to C, E, F, G, H, L, S, W or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position F35 to A, C, D, E, G, H, I, K, L, M, N, P, Q, R, S, T, V, W or Y, preferably to A, C, E, G, K, N, T or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1. In another aspect, the variant comprises a substitution at position D36 to A, C, E, F, G, H, I, K, L, M, N, P, Q, R, S, T, V, W or Y, preferably to H or Q, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A37 to C, D, E, F, G, H, I, K, L, M, N, P, Q, R, S, T, V, W or Y, preferably to N or Q, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position N38 to A, C, D, E, F, G, H, I, K, L, M, P, Q, R, S, T, V, W or Y, preferably to C, G or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position W39 to A, C, D, E, F, G, H, I, K, L, M, N, P, Q, R, S, T, V or Y, preferably to G or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position K40 to A, C, D, E, F, G, H, I, L, M, N, P, Q, R, S, T, V, W or Y, preferably to C, D, F, G, I, M, N, P or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position F41 to A, C, D, E, G, H, I, K, L, M, N, P, Q, R, S, T, V, W or Y, preferably to A, C, G, I, Q, S or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position M42 to A, C, D, E, F, G, H, I, K, L, N, P, Q, R, S, T, V, W or Y, preferably to E, N or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position L43 to A, C, D, E, F, G, H, I, K, M, N, P, Q, R, S, T, V, W or Y, preferably to A, C, G, I, S, T or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position S44 to A, C, D, E, F, G, H, I, K, L, M, N, P, Q, R, T, V, W or Y, preferably to C, M, N or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position D45 to A, C, E, F, G, H, I, K, L, M, N, P, Q, R, S, T, V, W or Y, preferably to A, L, P or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V47 to A, C, D, E, F, G, H, I, K, L, M, N, P, Q, R, S, T, W or Y, preferably to K or R, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position Q48 to A, C, D, E, F, G, H, I, K, L, M, N, P, R, S, T, V, W or Y, preferably to S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A49 to C, D, E, F, G, H, I, K, L, M, N, P, Q, R, S, T, V, W or Y, preferably to C, D, H, R, S, T or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position D51 to A, C, E, F, G, H, I, K, L, M, N, P, Q, R, S, T, V, W or Y, preferably to G, I, K, M, P or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A53 to C, D, E, F, G, H, I, K, L, M, N, P, Q, R, S, T, V, W or Y, preferably to C, G, L, R, S, T, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position F54 to A, C, D, E, G, H, I, K, L, M, N, P, Q, R, S, T, V, W or Y, preferably to M or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position D55 to A, C, E, F, G, H, I, K, L, M, N, P, Q, R, S, T, V, W or Y, preferably to C, F, G, H, M, N, P, S or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position S57 to A, C, D, E, F, G, H, I, K, L, M, N, P, Q, R, T, V, W or Y, preferably to A, C, E or G, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A58 to C, D, E, F, G, H, I, K, L, M, N, P, Q, R, S, T, V, W or Y, preferably to D, G, I, M, N, Q, R or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position W59 to A, C, D, E, F, G, H, I, K, L, M, N, P, Q, R, S, T, V or Y, preferably to D, I, K, L, N, P or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position Q60 to A, C, D, E, F, G, H, I, K, L, M, N, P, R, S, T, V, W or Y, preferably to A, C, E, F, G, K, L, M, R, S, V or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position Q61 to A, C, D, E, F, G, H, I, K, L, M, N, P, R, S, T, V, W or Y, preferably to K, P or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V62 to A, C, D, E, F, G, H, I, K, L, M, N, P, Q, R, S, T, W or Y, preferably to G, N, S, T or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position D63 to A, C, E, F, G, H, I, K, L, M, N, P, Q, R, S, T, V, W or Y, preferably to G, L, P, S or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position L64 to A, C, D, E, F, G, H, I, K, M, N, P, Q, R, S, T, V, W or Y, preferably to E or G, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position H66 to A, C, D, E, F, G, I, K, L, M, N, P, Q, R, S, T, V, W or Y, preferably to C, L, R, T, W or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position D67 to A, C, E, F, G, H, I, K, L, M, N, P, Q, R, S, T, V, W or Y, preferably to E, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position Y68 to A, C, D, E, F, G, H, I, K, L, M, N, P, Q, R, S, T, V or W, preferably to P or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position 170 to A, C, D, E, F, G, H, K, L, M, N, P, Q, R, S, T, V, W or Y, preferably to A, H, K, P or R, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T71 to A, C, D, E, F, G, H, I, K, L, M, N, P, Q, R, S, V, W or Y, preferably to C, E, G, H, K, L, N, P, Q, R or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1. In another aspect, the variant comprises a substitution at position K73 to A, C, D, E, F, G, H, I, L, M, N, P, Q, R, S, T, V, W or Y, preferably to A, D, F, G, Q or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position Y74 to A, C, D, E, F, G, H, I, K, L, M, N, P, Q, R, S, T, V or W, preferably to G, K, T or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position S75 to A, C, D, E, F, G, H, I, K, L, M, N, P, Q, R, T, V, W or Y, preferably to G, L, Q, R or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position Q76 to A, C, D, E, F, G, H, I, K, L, M, N, P, R, S, T, V, W or Y, preferably to G, I, K, M, P, S, V or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1. In another aspect, the variant comprises a substitution at position S77 to A, C, D, E, F, G, H, I, K, L, M, N, P, Q, R, T, V, W or Y, preferably to C, D, E, F, G, H, I, K, L, M, R, T, V, W or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position N78 to A, C, D, E, F, G, H, I, K, L, M, P, Q, R, S, T, V, W or Y, preferably to C, E, F, K, Q, R, S or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position E79 to A, C, D, F, G, H, I, K, L, M, N, P, Q, R, S, T, V, W or Y, preferably to H, Q, S, T or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A80 to C, D, E, F, G, H, I, K, L, M, N, P, Q, R, S, T, V, W or Y, preferably to K, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position E81 to A, C, D, F, G, H, I, K, L, M, N, P, Q, R, S, T, V, W or Y, preferably to A or Q, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A83 to C, D, E, F, G, H, I, K, L, M, N, P, Q, R, S, T, V, W or Y, preferably to E or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position L85 to A, C, D, E, F, G, H, I, K, M, N, P, Q, R, S, T, V, W or Y, preferably to A, C, D, F, M, N, S, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1. In another aspect, the variant comprises a substitution at position P86 to A, C, D, E, F, G, H, I, K, L, M, N, Q, R, S, T, V, W or Y, preferably to E, G, H, N, Q, R, V, W or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1. In another aspect, the variant comprises a substitution at position G87 to A, C, D, E, F, H, I, K, L, M, N, P, Q, R, S, T, V, W or Y, preferably to A, D, E, N or Q, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G88 to A, C, D, E, F, H, I, K, L, M, N, P, Q, R, S, T, V, W or Y, preferably to A, F, I, M, Q or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T89 to A, C, D, E, F, G, H, I, K, L, M, N, P, Q, R, S, V, W or Y, preferably to C, G, H, K, L, M, N, P, W or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G90 to A, C, D, E, F, H, I, K, L, M, N, P, Q, R, S, T, V, W or Y, preferably to A, C, D, L, S, T or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position W91 to A, C, D, E, F, G, H, I, K, L, M, N, P, Q, R, S, T, V or Y, preferably to E, L, P, Q, R, S or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1. In another aspect, the variant comprises a substitution at position Y92 to A, C, D, E, F, G, H, I, K, L, M, N, P, Q, R, S, T, V or W, preferably to F, H, I, M, N, S, T, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1. In another aspect, the variant comprises a substitution at position R93 to A, C, D, E, F, G, H, I, K, L, M, N, P, Q, S, T, V, W or Y, preferably to A, D, F, H, I, L, N, T, V or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position K94 to A, C, D, E, F, G, H, I, L, M, N, P, Q, R, S, T, V, W or Y, preferably to A, C, G, R, S, T or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position S95 to A, C, D, E, F, G, H, I, K, L, M, N, P, Q, R, T, V, W or Y, preferably to A, C, D, E, G, I, L, Q or R, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1. In another aspect, the variant comprises a substitution at position F96 to A, C, D, E, G, H, I, K, L, M, N, P, Q, R, S, T, V, W or Y, preferably to A, C, I, K, L, M, P, S, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1. In another aspect, the variant comprises a substitution at position T97 to A, C, D, E, F, G, H, I, K, L, M, N, P, Q, R, S, V, W or Y, preferably to F, S or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position 198 to A, C, D, E, F, G, H, K, L, M, N, P, Q, R, S, T, V, W or Y, preferably to C, H, S or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position R100 to A, C, D, E, F, G, H, I, K, L, M, N, P, Q, S, T, V, W or Y, preferably to T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position D101 to A, C, E, F, G, H, I, K, L, M, N, P, Q, R, S, T, V, W or Y, preferably to A, P or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position L102 to A, C, D, E, F, G, H, I, K, M, N, P, Q, R, S, T, V, W or Y, preferably to A, G, M, P or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G104 to A, C, D, E, F, H, I, K, L, M, N, P, Q, R, S, T, V, W or Y, preferably to C, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position K105 to A, C, D, E, F, G, H, I, L, M, N, P, Q, R, S, T, V, W or Y, preferably to D, Q, R, W or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1. In another aspect, the variant comprises a substitution at position R106 to A, C, D, E, F, G, H, I, K, L, M, N, P, Q, S, T, V, W or Y, preferably to K, P, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position 1107 to A, C, D, E, F, G, H, K, L, M, N, P, Q, R, S, T, V, W or Y, preferably to A, F, G, Q or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A108 to C, D, E, F, G, H, I, K, L, M, N, P, Q, R, S, T, V, W or Y, preferably to E, S or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position 1109 to A, C, D, E, F, G, H, K, L, M, N, P, Q, R, S, T, V, W or Y, preferably to M or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position N110 to A, C, D, E, F, G, H, I, K, L, M, P, Q, R, S, T, V, W or Y, preferably to A, F, S, T, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position F111 to A, C, D, E, G, H, I, K, L, M, N, P, Q, R, S, T, V, W or Y, preferably to A, C, L, Q or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position D112 to A, C, E, F, G, H, I, K, L, M, N, P, Q, R, S, T, V, W or Y, preferably to A, F, G or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G113 to A, C, D, E, F, H, I, K, L, M, N, P, Q, R, S, T, V, W or Y, preferably to A or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V114 to A, C, D, E, F, G, H, I, K, L, M, N, P, Q, R, S, T, W or Y, preferably to F, G, M or R, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position Y115 to A, C, D, E, F, G, H, I, K, L, M, N, P, Q, R, S, T, V or W, preferably to E, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position M116 to A, C, D, E, F, G, H, I, K, L, N, P, Q, R, S, T, V, W or Y, preferably to A, C, D, W or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position N117 to A, C, D, E, F, G, H, I, K, L, M, P, Q, R, S, T, V, W or Y, preferably to K, R, T or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A118 to C, D, E, F, G, H, I, K, L, M, N, P, Q, R, S, T, V, W or Y, preferably to K, P or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T119 to A, C, D, E, F, G, H, I, K, L, M, N, P, Q, R, S, V, W or Y, preferably to A, G or L, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V120 to A, C, D, E, F, G, H, I, K, L, M, N, P, Q, R, S, T, W or Y, preferably to A or K, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position W121 to A, C, D, E, F, G, H, I, K, L, M, N, P, Q, R, S, T, V or Y, preferably to C, D, R, T, V or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position F122 to A, C, D, E, G, H, I, K, L, M, N, P, Q, R, S, T, V, W or Y, preferably to A, M, S or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position N123 to A, C, D, E, F, G, H, I, K, L, M, P, Q, R, S, T, V, W or Y, preferably to P, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G124 to A, C, D, E, F, H, I, K, L, M, N, P, Q, R, S, T, V, W or Y, preferably to E, M, Q or R, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V125 to A, C, D, E, F, G, H, I, K, L, M, N, P, Q, R, S, T, W or Y, preferably to D, E or I, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position K126 to A, C, D, E, F, G, H, I, L, M, N, P, Q, R, S, T, V, W or Y, preferably to E or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G128 to A, C, D, E, F, H, I, K, L, M, N, P, Q, R, S, T, V, W or Y, preferably to A or D, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T129 to A, C, D, E, F, G, H, I, K, L, M, N, P, Q, R, S, V, W or Y, preferably to E or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position H130 to A, C, D, E, F, G, I, K, L, M, N, P, Q, R, S, T, V, W or Y, preferably to A, C, Q, S or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position P131 to A, C, D, E, F, G, H, I, K, L, M, N, Q, R, S, T, V, W or Y, preferably to K, L or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position Y132 to A, C, D, E, F, G, H, I, K, L, M, N, P, Q, R, S, T, V or W, preferably to C, E or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G133 to A, C, D, E, F, H, I, K, L, M, N, P, Q, R, S, T, V, W or Y, preferably to E, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position S135 to A, C, D, E, F, G, H, I, K, L, M, N, P, Q, R, T, V, W or Y, preferably to E, P or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position P136 to A, C, D, E, F, G, H, I, K, L, M, N, Q, R, S, T, V, W or Y, preferably to R or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position F137 to A, C, D, E, G, H, I, K, L, M, N, P, Q, R, S, T, V, W or Y, preferably to A, C, D, G, L or P, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position S138 to A, C, D, E, F, G, H, I, K, L, M, N, P, Q, R, T, V, W or Y, preferably to A, D, G, H, L, M, R or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position F139 to A, C, D, E, G, H, I, K, L, M, N, P, Q, R, S, T, V, W or Y, preferably to A, E, Q or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position D140 to A, C, E, F, G, H, I, K, L, M, N, P, Q, R, S, T, V, W or Y, preferably to G, L or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position L141 to A, C, D, E, F, G, H, I, K, M, N, P, Q, R, S, T, V, W or Y, preferably to G or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T142 to A, C, D, E, F, G, H, I, K, L, M, N, P, Q, R, S, V, W or Y, preferably to E, S or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position K146 to A, C, D, E, F, G, H, I, L, M, N, P, Q, R, S, T, V, W or Y, preferably to A, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G148 to A, C, D, E, F, H, I, K, L, M, N, P, Q, R, S, T, V, W or Y, preferably to H, K or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G149 to A, C, D, E, F, H, I, K, L, M, N, P, Q, R, S, T, V, W or Y, preferably to E, H, I, M, Q or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position E150 to A, C, D, F, G, H, I, K, L, M, N, P, Q, R, S, T, V, W or Y, preferably to A, C, G, L, N or R, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position N151 to A, C, D, E, F, G, H, I, K, L, M, P, Q, R, S, T, V, W or Y, preferably to L, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position 1153 to A, C, D, E, F, G, H, K, L, M, N, P, Q, R, S, T, V, W or Y, preferably to A or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V154 to A, C, D, E, F, G, H, I, K, L, M, N, P, Q, R, S, T, W or Y, preferably to E, I, K, L, M or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V155 to A, C, D, E, F, G, H, I, K, L, M, N, P, Q, R, S, T, W or Y, preferably to F, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V157 to A, C, D, E, F, G, H, I, K, L, M, N, P, Q, R, S, T, W or Y, preferably to A, G, L, P, Q or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1. In another aspect, the variant comprises a substitution at position E158 to A, C, D, F, G, H, I, K, L, M, N, P, Q, R, S, T, V, W or Y, preferably to G, H, K, Q or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position N159 to A, C, D, E, F, G, H, I, K, L, M, P, Q, R, S, T, V, W or Y, preferably to D, H, S or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position R160 to A, C, D, E, F, G, H, I, K, L, M, N, P, Q, S, T, V, W or Y, preferably to G, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position L161 to A, C, D, E, F, G, H, I, K, M, N, P, Q, R, S, T, V, W or Y, preferably to E, K, M, S or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position P162 to A, C, D, E, F, G, H, I, K, L, M, N, Q, R, S, T, V, W or Y, preferably to F, G, N, T or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position S164 to A, C, D, E, F, G, H, I, K, L, M, N, P, Q, R, T, V, W or Y, preferably to A, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position R165 to A, C, D, E, F, G, H, I, K, L, M, N, P, Q, S, T, V, W or Y, preferably to H, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position W166 to A, C, D, E, F, G, H, I, K, L, M, N, P, Q, R, S, T, V or Y, preferably to S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position Y167 to A, C, D, E, F, G, H, I, K, L, M, N, P, Q, R, S, T, V or W, preferably to A or C, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position S168 to A, C, D, E, F, G, H, I, K, L, M, N, P, Q, R, T, V, W or Y, preferably to C, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G169 to A, C, D, E, F, H, I, K, L, M, N, P, Q, R, S, T, V, W or Y, preferably to A, C, D or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position S170 to A, C, D, E, F, G, H, I, K, L, M, N, P, Q, R, T, V, W or Y, preferably to L or Q, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G171 to A, C, D, E, F, H, I, K, L, M, N, P, Q, R, S, T, V, W or Y, preferably to C, F or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position 1172 to A, C, D, E, F, G, H, K, L, M, N, P, Q, R, S, T, V, W or Y, preferably to G, K, P or Q, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position Y173 to A, C, D, E, F, G, H, I, K, L, M, N, P, Q, R, S, T, V or W, preferably to A, H, M, P, S or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position R174 to A, C, D, E, F, G, H, I, K, L, M, N, P, Q, S, T, V, W or Y, preferably to E or K, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position D175 to A, C, E, F, G, H, I, K, L, M, N, P, Q, R, S, T, V, W or Y, preferably to E or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V176 to A, C, D, E, F, G, H, I, K, L, M, N, P, Q, R, S, T, W or Y, preferably to E, K or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T177 to A, C, D, E, F, G, H, I, K, L, M, N, P, Q, R, S, V, W or Y, preferably to A, C, E, K or L, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position L178 to A, C, D, E, F, G, H, I, K, M, N, P, Q, R, S, T, V, W or Y, preferably to I, Q or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T179 to A, C, D, E, F, G, H, I, K, L, M, N, P, Q, R, S, V, W or Y, preferably to A, C, D, H, I, K, L, N, P or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1. In another aspect, the variant comprises a substitution at position V180 to A, C, D, E, F, G, H, I, K, L, M, N, P, Q, R, S, T, W or Y, preferably to A, C, D, E, G or M, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T181 to A, C, D, E, F, G, H, I, K, L, M, N, P, Q, R, S, V, W or Y, preferably to A, D, F, K or R, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1. In another aspect, the variant comprises a substitution at position D182 to A, C, E, F, G, H, I, K, L, M, N, P, Q, R, S, T, V, W or Y, preferably to F, L or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G183 to A, C, D, E, F, H, I, K, L, M, N, P, Q, R, S, T, V, W or Y, preferably to W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V184 to A, C, D, E, F, G, H, I, K, L, M, N, P, Q, R, S, T, W or Y, preferably to F, H, P, Q, R, S or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1. In another aspect, the variant comprises a substitution at position H185 to A, C, D, E, F, G, I, K, L, M, N, P, Q, R, S, T, V, W or Y, preferably to G, L or R, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V186 to A, C, D, E, F, G, H, I, K, L, M, N, P, Q, R, S, T, W or Y, preferably to A, E, G or N, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G187 to A, C, D, E, F, H, I, K, L, M, N, P, Q, R, S, T, V, W or Y, preferably to A, D or H, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position N188 to A, C, D, E, F, G, H, I, K, L, M, P, Q, R, S, T, V, W or Y, preferably to E, R, S, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position N189 to A, C, D, E, F, G, H, I, K, L, M, P, Q, R, S, T, V, W or Y, preferably to A or E, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G190 to A, C, D, E, F, H, I, K, L, M, N, P, Q, R, S, T, V, W or Y, preferably to C, F, H, Q or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V191 to A, C, D, E, F, G, H, I, K, L, M, N, P, Q, R, S, T, W or Y, preferably to C, T, W or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position 1193 to A, C, D, E, F, G, H, K, L, M, N, P, Q, R, S, T, V, W or Y, preferably to N, Q, T or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position K194 to A, C, D, E, F, G, H, I, L, M, N, P, Q, R, S, T, V, W or Y, preferably to A, I, L or R, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T195 to A, C, D, E, F, G, H, I, K, L, M, N, P, Q, R, S, V, W or Y, preferably to A, E, M, S or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position P196 to A, C, D, E, F, G, H, I, K, L, M, N, Q, R, S, T, V, W or Y, preferably to A, H, I, M, S or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position S197 to A, C, D, E, F, G, H, I, K, L, M, N, P, Q, R, T, V, W or Y, preferably to A, C, E, K, L or P, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position L198 to A, C, D, E, F, G, H, I, K, M, N, P, Q, R, S, T, V, W or Y, preferably to E, F, H, I, K, R, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A199 to C, D, E, F, G, H, I, K, L, M, N, P, Q, R, S, T, V, W or Y, preferably to E, F, K, P, R or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1. In another aspect, the variant comprises a substitution at position Q201 to A, C, D, E, F, G, H, I, K, L, M, N, P, R, S, T, V, W or Y, preferably to C, E, I, K, M or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1. In another aspect, the variant comprises a substitution at position N202 to A, C, D, E, F, G, H, I, K, L, M, P, Q, R, S, T, V, W or Y, preferably to A, D, F, G, K, L, M, Q, R, S, T or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G203 to A, C, D, E, F, H, I, K, L, M, N, P, Q, R, S, T, V, W or Y, preferably to C, K, M, Q, R, S, V, W or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G204 to A, C, D, E, F, H, I, K, L, M, N, P, Q, R, S, T, V, W or Y, preferably to A, C, D, K, R, S or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position N205 to A, C, D, E, F, G, H, I, K, L, M, P, Q, R, S, T, V, W or Y, preferably to E, G, H, L, P, W or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1. In another aspect, the variant comprises a substitution at position V206 to A, C, D, E, F, G, H, I, K, L, M, N, P, Q, R, S, T, W or Y, preferably to A, C, D, F, G, I, K, Q, R, S or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1. In another aspect, the variant comprises a substitution at position T207 to A, C, D, E, F, G, H, I, K, L, M, N, P, Q, R, S, V, W or Y, preferably to A, C, G, I, K, L, M, N, Q, R or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position M208 to A, C, D, E, F, G, H, I, K, L, N, P, Q, R, S, T, V, W or Y, preferably to A, S or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position N209 to A, C, D, E, F, G, H, I, K, L, M, P, Q, R, S, T, V, W or Y, preferably to C, D, G, K, L, Q, R or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position L210 to A, C, D, E, F, G, H, I, K, M, N, P, Q, R, S, T, V, W or Y, preferably to A, C, F, G, H, I, Q, R, S, T or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1. In another aspect, the variant comprises a substitution at position T211 to A, C, D, E, F, G, H, I, K, L, M, N, P, Q, R, S, V, W or Y, preferably to A, D, E, F, K, N, Q, R or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T212 to A, C, D, E, F, G, H, I, K, L, M, N, P, Q, R, S, V, W or Y, preferably to A, C, E, F, G, H, K, L, M, S or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ

In another aspect, the variant comprises a substitution at position K213 to A, C, D, E, F, G, H, I, L, M, N, P, Q, R, S, T, V, W or Y, preferably to A, C, D, F, I, L, M, N, Q, R, S, T, V or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V214 to A, C, D, E, F, G, H, I, K, L, M, N, P, Q, R, S, T, W or Y, preferably to A, C, T or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A215 to C, D, E, F, G, H, I, K, L, M, N, P, Q, R, S, T, V, W or Y, preferably to D, E, F, I, K, L, Q, R, S or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1. In another aspect, the variant comprises a substitution at position N216 to A, C, D, E, F, G, H, I, K, L, M, P, Q, R, S, T, V, W or Y, preferably to D, T or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position D217 to A, C, E, F, G, H, I, K, L, M, N, P, Q, R, S, T, V, W or Y, preferably to F, G, L, M, T or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T218 to A, C, D, E, F, G, H, I, K, L, M, N, P, Q, R, S, V, W or Y, preferably to D, G or H, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position K219 to A, C, D, E, F, G, H, I, L, M, N, P, Q, R, S, T, V, W or Y, preferably to A, C, F, H or M, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A220 to C, D, E, F, G, H, I, K, L, M, N, P, Q, R, S, T, V, W or Y, preferably to C, G, I, L, M, T, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A221 to C, D, E, F, G, H, I, K, L, M, N, P, Q, R, S, T, V, W or Y, preferably to C, D, E, L, N, R, V or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A222 to C, D, E, F, G, H, I, K, L, M, N, P, Q, R, S, T, V, W or Y, preferably to D, I, L, P, R, W or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position N223 to A, C, D, E, F, G, H, I, K, L, M, P, Q, R, S, T, V, W or Y, preferably to A, E, F, G, K, L, M, R, S, T, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position 1224 to A, C, D, E, F, G, H, K, L, M, N, P, Q, R, S, T, V, W or Y, preferably to G or Q, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T225 to A, C, D, E, F, G, H, I, K, L, M, N, P, Q, R, S, V, W or Y, preferably to A, G or L, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position L226 to A, C, D, E, F, G, H, I, K, M, N, P, Q, R, S, T, V, W or Y, preferably to C, M or Q, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position K227 to A, C, D, E, F, G, H, I, L, M, N, P, Q, R, S, T, V, W or Y, preferably to T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position Q228 to A, C, D, E, F, G, H, I, K, L, M, N, P, R, S, T, V, W or Y, preferably to N or R, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T229 to A, C, D, E, F, G, H, I, K, L, M, N, P, Q, R, S, V, W or Y, preferably to A, C, D, G, H, M, N, Q, R or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1. In another aspect, the variant comprises a substitution at position V230 to A, C, D, E, F, G, H, I, K, L, M, N, P, Q, R, S, T, W or Y, preferably to F, L, M, Q, R or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position F231 to A, C, D, E, G, H, I, K, L, M, N, P, Q, R, S, T, V, W or Y, preferably to A, E, G, I, K, L, Q, S, V, W or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1. In another aspect, the variant comprises a substitution at position P232 to A, C, D, E, F, G, H, I, K, L, M, N, Q, R, S, T, V, W or Y, preferably to G, H, L, M, R, S, T, V, W or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position K233 to A, C, D, E, F, G, H, I, L, M, N, P, Q, R, S, T, V, W or Y, preferably to A, C, E, F, G, L, P, R, S, V, W or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G234 to A, C, D, E, F, H, I, K, L, M, N, P, Q, R, S, T, V, W or Y, preferably to A, C, D, E, K, L, Q, R, V, W or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G235 to A, C, D, E, F, H, I, K, L, M, N, P, Q, R, S, T, V, W or Y, preferably to C, F, H, I, K, M, Q, R, T, W or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position K236 to A, C, D, E, F, G, H, I, L, M, N, P, Q, R, S, T, V, W or Y, preferably to A, D, E, G, L, M, P, R, S, T, W or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ

In another aspect, the variant comprises a substitution at position T237 to A, C, D, E, F, G, H, I, K, L, M, N, P, Q, R, S, V, W or Y, preferably to D, F, I, K, M, Q, R, S, V or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position D238 to A, C, E, F, G, H, I, K, L, M, N, P, Q, R, S, T, V, W or Y, preferably to A, E, F, G, H, I, K, L, M, N, P, Q or R, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A239 to C, D, E, F, G, H, I, K, L, M, N, P, Q, R, S, T, V, W or Y, preferably to C, E, G, I, K or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A240 to C, D, E, F, G, H, I, K, L, M, N, P, Q, R, S, T, V, W or Y, preferably to C, E, L, P, Q, T, V, W or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position 1241 to A, C, D, E, F, G, H, K, L, M, N, P, Q, R, S, T, V, W or Y, preferably to T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G242 to A, C, D, E, F, H, I, K, L, M, N, P, Q, R, S, T, V, W or Y, preferably to K, L, M, P, T or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1. In another aspect, the variant comprises a substitution at position T243 to A, C, D, E, F, G, H, I, K, L, M, N, P, Q, R, S, V, W or Y, preferably to I, M, R or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V244 to A, C, D, E, F, G, H, I, K, L, M, N, P, Q, R, S, T, W or Y, preferably to A, E, G, L or R, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T245 to A, C, D, E, F, G, H, I, K, L, M, N, P, Q, R, S, V, W or Y, preferably to E, G, L, M, N, Q, R or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T246 to A, C, D, E, F, G, H, I, K, L, M, N, P, Q, R, S, V, W or Y, preferably to D, E, G, K or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A247 to C, D, E, F, G, H, I, K, L, M, N, P, Q, R, S, T, V, W or Y, preferably to D, E, K, N, P, Q, R, S, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1. In another aspect, the variant comprises a substitution at position S248 to A, C, D, E, F, G, H, I, K, L, M, N, P, Q, R, T, V, W or Y, preferably to A, E, F, H, I, L, Q, T or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position K249 to A, C, D, E, F, G, H, I, L, M, N, P, Q, R, S, T, V, W or Y, preferably to A, D, G, H, I, L, M, N, P, Q, S, T, V or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position S250 to A, C, D, E, F, G, H, I, K, L, M, N, P, Q, R, T, V, W or Y, preferably to H, M or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position 1251 to A, C, D, E, F, G, H, K, L, M, N, P, Q, R, S, T, V, W or Y, preferably to F, L, V, W or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A252 to C, D, E, F, G, H, I, K, L, M, N, P, Q, R, S, T, V, W or Y, preferably to C, E, F, H, I, P, R, S, W or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1. In another aspect, the variant comprises a substitution at position G254 to A, C, D, E, F, H, I, K, L, M, N, P, Q, R, S, T, V, W or Y, preferably to D, F, I, L, M, Q, R or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A255 to C, D, E, F, G, H, I, K, L, M, N, P, Q, R, S, T, V, W or Y, preferably to C, F, K, L, M, S, T, W or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position S256 to A, C, D, E, F, G, H, I, K, L, M, N, P, Q, R, T, V, W or Y, preferably to A, C, F, G, K, L, M, N, Q, R, V, W or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A257 to C, D, E, F, G, H, I, K, L, M, N, P, Q, R, S, T, V, W or Y, preferably to D, G, I, N, T or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position D258 to A, C, E, F, G, H, I, K, L, M, N, P, Q, R, S, T, V, W or Y, preferably to A, L, M or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V259 to A, C, D, E, F, G, H, I, K, L, M, N, P, Q, R, S, T, W or Y, preferably to E, L, S or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T260 to A, C, D, E, F, G, H, I, K, L, M, N, P, Q, R, S, V, W or Y, preferably to A, D, G, I, K or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1. In another aspect, the variant comprises a substitution at position S261 to A, C, D, E, F, G, H, I, K, L, M, N, P, Q, R, T, V, W or Y, preferably to A, D, H, R, W or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1. In another aspect, the variant comprises a substitution at position T262 to A, C, D, E, F, G, H, I, K, L, M, N, P, Q, R, S, V, W or Y, preferably to D, E, F, G, H, L, P or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position 1263 to A, C, D, E, F, G, H, K, L, M, N, P, Q, R, S, T, V, W or Y, preferably to A, C, G, L, S or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T264 to A, C, D, E, F, G, H, I, K, L, M, N, P, Q, R, S, V, W or Y, preferably to F, G, K, L, M, P, Q, R, S or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1. In another aspect, the variant comprises a substitution at position A265 to C, D, E, F, G, H, I, K, L, M, N, P, Q, R, S, T, V, W or Y, preferably to G, I, K, R, S or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A266 to C, D, E, F, G, H, I, K, L, M, N, P, Q, R, S, T, V, W or Y, preferably to D, E, G, K, L, M, P, Q, S or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position S267 to A, C, D, E, F, G, H, I, K, L, M, N, P, Q, R, T, V, W or Y, preferably to A, D, K, M, N, P, Q, R or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1. In another aspect, the variant comprises a substitution at position P268 to A, C, D, E, F, G, H, I, K, L, M, N, Q, R, S, T, V, W or Y, preferably to F, G, M, R, V, W or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position K269 to A, C, D, E, F, G, H, I, L, M, N, P, Q, R, S, T, V, W or Y, preferably to G, R, V or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position L270 to A, C, D, E, F, G, H, I, K, M, N, P, Q, R, S, T, V, W or Y, preferably to D, M, N, R or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1. In another aspect, the variant comprises a substitution at position W271 to A, C, D, E, F, G, H, I, K, L, M, N, P, Q, R, S, T, V or Y, preferably to T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position S272 to A, C, D, E, F, G, H, I, K, L, M, N, P, Q, R, T, V, W or Y, preferably to E, G, K, L, N, T or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position 1273 to A, C, D, E, F, G, H, K, L, M, N, P, Q, R, S, T, V, W or Y, preferably to K, L, P, R, S or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position K274 to A, C, D, E, F, G, H, I, L, M, N, P, Q, R, S, T, V, W or Y, preferably to D, P, Q or R, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position N275 to A, C, D, E, F, G, H, I, K, L, M, P, Q, R, S, T, V, W or Y, preferably to K, M, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position N277 to A, C, D, E, F, G, H, I, K, L, M, P, Q, R, S, T, V, W or Y, preferably to F, K or R, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position L278 to A, C, D, E, F, G, H, I, K, M, N, P, Q, R, S, T, V, W or Y, preferably to A, G, H, I, K, M, P, Q, R, S or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position Y279 to A, C, D, E, F, G, H, I, K, L, M, N, P, Q, R, S, T, V or W, preferably to M, T or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T280 to A, C, D, E, F, G, H, I, K, L, M, N, P, Q, R, S, V, W or Y, preferably to A, D, E, F, H, M or Q, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V281 to A, C, D, E, F, G, H, I, K, L, M, N, P, Q, R, S, T, W or Y, preferably to A, I, L or Q, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position R282 to A, C, D, E, F, G, H, I, K, L, M, N, P, Q, S, T, V, W or Y, preferably to E, F, H, I, K, N, S, T, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T283 to A, C, D, E, F, G, H, I, K, L, M, N, P, Q, R, S, V, W or Y, preferably to M, R or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position E284 to A, C, D, F, G, H, I, K, L, M, N, P, Q, R, S, T, V, W or Y, preferably to A, D, F, H, L, M, N, Q, R or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V285 to A, C, D, E, F, G, H, I, K, L, M, N, P, Q, R, S, T, W or Y, preferably to H, I or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position L286 to A, C, D, E, F, G, H, I, K, M, N, P, Q, R, S, T, V, W or Y, preferably to A, C, D, F, N, R, T, W or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position N287 to A, C, D, E, F, G, H, I, K, L, M, P, Q, R, S, T, V, W or Y, preferably to I or L, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G288 to A, C, D, E, F, H, I, K, L, M, N, P, Q, R, S, T, V, W or Y, preferably to F, L or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V291 to A, C, D, E, F, G, H, I, K, L, M, N, P, Q, R, S, T, W or Y, preferably to C, D, F, G, H, L, P, S, T or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position L292 to A, C, D, E, F, G, H, I, K, M, N, P, Q, R, S, T, V, W or Y, preferably to A, D, E, H, Q, S or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position D293 to A, C, E, F, G, H, I, K, L, M, N, P, Q, R, S, T, V, W or Y, preferably to C, E, F, I, S, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T294 to A, C, D, E, F, G, H, I, K, L, M, N, P, Q, R, S, V, W or Y, preferably to C, G, M, Q or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position Y295 to A, C, D, E, F, G, H, I, K, L, M, N, P, Q, R, S, T, V or W, preferably to F, G, I, L, R, S or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1. In another aspect, the variant comprises a substitution at position D296 to A, C, E, F, G, H, I, K, L, M, N, P, Q, R, S, T, V, W or Y, preferably to F, H, K, L, N, R or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1. In another aspect, the variant comprises a substitution at position T297 to A, C, D, E, F, G, H, I, K, L, M, N, P, Q, R, S, V, W or Y, preferably to G, I, Q, R or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1. In another aspect, the variant comprises a substitution at position E298 to A, C, D, F, G, H, I, K, L, M, N, P, Q, R, S, T, V, W or Y, preferably to G, I, L, M, R or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position Y299 to A, C, D, E, F, G, H, I, K, L, M, N, P, Q, R, S, T, V or W, preferably to F or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position F301 to A, C, D, E, G, H, I, K, L, M, N, P, Q, R, S, T, V, W or Y, preferably to C or L, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position R302 to A, C, D, E, F, G, H, I, K, L, M, N, P, Q, S, T, V, W or Y, preferably to E, I, K, M, N or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position W303 to A, C, D, E, F, G, H, I, K, L, M, N, P, Q, R, S, T, V or Y, preferably to A, C, D, F, S or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T304 to A, C, D, E, F, G, H, I, K, L, M, N, P, Q, R, S, V, W or Y, preferably to D, E, I, K, P, S or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G305 to A, C, D, E, F, H, I, K, L, M, N, P, Q, R, S, T, V, W or Y, preferably to E, F, L, M, N, P, T or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position F306 to A, C, D, E, G, H, I, K, L, M, N, P, Q, R, S, T, V, W or Y, preferably to D, K, L or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position D307 to A, C, E, F, G, H, I, K, L, M, N, P, Q, R, S, T, V, W or Y, preferably to A, E, F, G, L, M, N, Q, S, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A308 to C, D, E, F, G, H, I, K, L, M, N, P, Q, R, S, T, V, W or Y, preferably to C, E, G, I, M or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T309 to A, C, D, E, F, G, H, I, K, L, M, N, P, Q, R, S, V, W or Y, preferably to C, D, E, I, K, M, S or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position S310 to A, C, D, E, F, G, H, I, K, L, M, N, P, Q, R, T, V, W or Y, preferably to A, C, G, H, I, L, M, N, R or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G311 to A, C, D, E, F, H, I, K, L, M, N, P, Q, R, S, T, V, W or Y, preferably to E, F, L, Q, R or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position F312 to A, C, D, E, G, H, I, K, L, M, N, P, Q, R, S, T, V, W or Y, preferably to H, L, R or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position L314 to A, C, D, E, F, G, H, I, K, M, N, P, Q, R, S, T, V, W or Y, preferably to A, C, T, V or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position N315 to A, C, D, E, F, G, H, I, K, L, M, P, Q, R, S, T, V, W or Y, preferably to S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G316 to A, C, D, E, F, H, I, K, L, M, N, P, Q, R, S, T, V, W or Y, preferably to A, C, N, R, S, W or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1. In another aspect, the variant comprises a substitution at position E317 to A, C, D, F, G, H, I, K, L, M, N, P, Q, R, S, T, V, W or Y, preferably to D, H, I, K, V, W or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position K318 to A, C, D, E, F, G, H, I, L, M, N, P, Q, R, S, T, V, W or Y, preferably to C, E, F, G, H, L, M, N, P, Q or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ

In another aspect, the variant comprises a substitution at position K320 to A, C, D, E, F, G, H, I, L, M, N, P, Q, R, S, T, V, W or Y, preferably to C, E, F, I, L, R, T, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position L321 to A, C, D, E, F, G, H, I, K, M, N, P, Q, R, S, T, V, W or Y, preferably to C, G, H, M, V or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position K322 to A, C, D, E, F, G, H, I, L, M, N, P, Q, R, S, T, V, W or Y, preferably to C, F, I, N, P, Q, R or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1. In another aspect, the variant comprises a substitution at position V324 to A, C, D, E, F, G, H, I, K, L, M, N, P, Q, R, S, T, W or Y, preferably to E, G or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position S325 to A, C, D, E, F, G, H, I, K, L, M, N, P, Q, R, T, V, W or Y, preferably to D, G or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position M326 to A, C, D, E, F, G, H, I, K, L, N, P, Q, R, S, T, V, W or Y, preferably to E, G, S, T, V or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position H328 to A, C, D, E, F, G, I, K, L, M, N, P, Q, R, S, T, V, W or Y, preferably to C, D, F, G, I, L, M, R or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1. In another aspect, the variant comprises a substitution at position D329 to A, C, E, F, G, H, I, K, L, M, N, P, Q, R, S, T, V, W or Y, preferably to S or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G331 to A, C, D, E, F, H, I, K, L, M, N, P, Q, R, S, T, V, W or Y, preferably to V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A335 to C, D, E, F, G, H, I, K, L, M, N, P, Q, R, S, T, V, W or Y, preferably to C, G or L, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A337 to C, D, E, F, G, H, I, K, L, M, N, P, Q, R, S, T, V, W or Y, preferably to D, E, G, L or Q, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position N338 to A, C, D, E, F, G, H, I, K, L, M, P, Q, R, S, T, V, W or Y, preferably to P, R or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position R340 to A, C, D, E, F, G, H, I, K, L, M, N, P, Q, S, T, V, W or Y, preferably to A, C or L, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A341 to C, D, E, F, G, H, I, K, L, M, N, P, Q, R, S, T, V, W or Y, preferably to M or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position 1342 to A, C, D, E, F, G, H, K, L, M, N, P, Q, R, S, T, V, W or Y, preferably to A, G, K or P, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position E343 to A, C, D, F, G, H, I, K, L, M, N, P, Q, R, S, T, V, W or Y, preferably to A, H, N, R, S, T or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1. In another aspect, the variant comprises a substitution at position R344 to A, C, D, E, F, G, H, I, K, L, M, N, P, Q, S, T, V, W or Y, preferably to G, L or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position Q345 to A, C, D, E, F, G, H, I, K, L, M, N, P, R, S, T, V, W or Y, preferably to F, G, K, N or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V346 to A, C, D, E, F, G, H, I, K, L, M, N, P, Q, R, S, T, W or Y, preferably to A, C, F, I, L or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position E347 to A, C, D, F, G, H, I, K, L, M, N, P, Q, R, S, T, V, W or Y, preferably to A, C, D, F, I, R or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1. In another aspect, the variant comprises a substitution at position 1348 to A, C, D, E, F, G, H, K, L, M, N, P, Q, R, S, T, V, W or Y, preferably to A, D, G, M, Q or R, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position L349 to A, C, D, E, F, G, H, I, K, M, N, P, Q, R, S, T, V, W or Y, preferably to A, E, Q, S or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position Q350 to A, C, D, E, F, G, H, I, K, L, M, N, P, R, S, T, V, W or Y, preferably to G, N or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position K351 to A, C, D, E, F, G, H, I, L, M, N, P, Q, R, S, T, V, W or Y, preferably to N, R, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position M352 to A, C, D, E, F, G, H, I, K, L, N, P, Q, R, S, T, V, W or Y, preferably to G, L, T or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G353 to A, C, D, E, F, H, I, K, L, M, N, P, Q, R, S, T, V, W or Y, preferably to K, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V354 to A, C, D, E, F, G, H, I, K, L, M, N, P, Q, R, S, T, W or Y, preferably to E, G, S or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position N355 to A, C, D, E, F, G, H, I, K, L, M, P, Q, R, S, T, V, W or Y, preferably to H, M, R or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position 1357 to A, C, D, E, F, G, H, K, L, M, N, P, Q, R, S, T, V, W or Y, preferably to T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T359 to A, C, D, E, F, G, H, I, K, L, M, N, P, Q, R, S, V, W or Y, preferably to E or L, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T360 to A, C, D, E, F, G, H, I, K, L, M, N, P, Q, R, S, V, W or Y, preferably to V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position N362 to A, C, D, E, F, G, H, I, K, L, M, P, Q, R, S, T, V, W or Y, preferably to G, S or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position P363 to A, C, D, E, F, G, H, I, K, L, M, N, Q, R, S, T, V, W or Y, preferably to A, D, I, L, M, S or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A364 to C, D, E, F, G, H, I, K, L, M, N, P, Q, R, S, T, V, W or Y, preferably to C, E, F, G, I, M, P, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A365 to C, D, E, F, G, H, I, K, L, M, N, P, Q, R, S, T, V, W or Y, preferably to C, E, I, P, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position K366 to A, C, D, E, F, G, H, I, L, M, N, P, Q, R, S, T, V, W or Y, preferably to A, D, E, I, L, M, P, S or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A367 to C, D, E, F, G, H, I, K, L, M, N, P, Q, R, S, T, V, W or Y, preferably to C, I, N or Q, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position L368 to A, C, D, E, F, G, H, I, K, M, N, P, Q, R, S, T, V, W or Y, preferably to A, E, Q, S, V or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position 1369 to A, C, D, E, F, G, H, K, L, M, N, P, Q, R, S, T, V, W or Y, preferably to A, D, E, G, K, M, R, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1. In another aspect, the variant comprises a substitution at position D370 to A, C, E, F, G, H, I, K, L, M, N, P, Q, R, S, T, V, W or Y, preferably to C, L, Q, R, S or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V371 to A, C, D, E, F, G, H, I, K, L, M, N, P, Q, R, S, T, W or Y, preferably to D, F, G, I, L, Q or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1. In another aspect, the variant comprises a substitution at position C372 to A, D, E, F, G, H, I, K, L, M, N, P, Q, R, S, T, V, W or Y, preferably to P, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position N373 to A, C, D, E, F, G, H, I, K, L, M, P, Q, R, S, T, V, W or Y, preferably to G, L or R, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position E374 to A, C, D, F, G, H, I, K, L, M, N, P, Q, R, S, T, V, W or Y, preferably to K, L or R, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position K375 to A, C, D, E, F, G, H, I, L, M, N, P, Q, R, S, T, V, W or Y, preferably to D, I, N, Q or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G376 to A, C, D, E, F, H, I, K, L, M, N, P, Q, R, S, T, V, W or Y, preferably to A or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V377 to A, C, D, E, F, G, H, I, K, L, M, N, P, Q, R, S, T, W or Y, preferably to A, M or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position L378 to A, C, D, E, F, G, H, I, K, M, N, P, Q, R, S, T, V, W or Y, preferably to I, P, W or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V379 to A, C, D, E, F, G, H, I, K, L, M, N, P, Q, R, S, T, W or Y, preferably to A, C, M or N, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V380 to A, C, D, E, F, G, H, I, K, L, M, N, P, Q, R, S, T, W or Y, preferably to M, P or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position E381 to A, C, D, F, G, H, I, K, L, M, N, P, Q, R, S, T, V, W or Y, preferably to A, C, G, L, Q or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V383 to A, C, D, E, F, G, H, I, K, L, M, N, P, Q, R, S, T, W or Y, preferably to A, K or L, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position M386 to A, C, D, E, F, G, H, I, K, L, N, P, Q, R, S, T, V, W or Y, preferably to G, N, Q, S or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position W387 to A, C, D, E, F, G, H, I, K, L, M, N, P, Q, R, S, T, V or Y, preferably to H or L, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position N388 to A, C, D, E, F, G, H, I, K, L, M, P, Q, R, S, T, V, W or Y, preferably to A, E, L or R, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position R389 to A, C, D, E, F, G, H, I, K, L, M, N, P, Q, S, T, V, W or Y, preferably to A, C, E, K, M, N, Q, S, T or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1. In another aspect, the variant comprises a substitution at position S390 to A, C, D, E, F, G, H, I, K, L, M, N, P, Q, R, T, V, W or Y, preferably to C, D, G, H, N, P, Q, T or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position K391 to A, C, D, E, F, G, H, I, L, M, N, P, Q, R, S, T, V, W or Y, preferably to E, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position N392 to A, C, D, E, F, G, H, I, K, L, M, P, Q, R, S, T, V, W or Y, preferably to D, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G393 to A, C, D, E, F, H, I, K, L, M, N, P, Q, R, S, T, V, W or Y, preferably to A, E, N, R, S or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position N394 to A, C, D, E, F, G, H, I, K, L, M, P, Q, R, S, T, V, W or Y, preferably to L, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T395 to A, C, D, E, F, G, H, I, K, L, M, N, P, Q, R, S, V, W or Y, preferably to A, C, F, H, I, M, N, Q, S or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position E396 to A, C, D, F, G, H, I, K, L, M, N, P, Q, R, S, T, V, W or Y, preferably to K, L, M, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position Y398 to A, C, D, E, F, G, H, I, K, L, M, N, P, Q, R, S, T, V or W, preferably to M or N, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G399 to A, C, D, E, F, H, I, K, L, M, N, P, Q, R, S, T, V, W or Y, preferably to S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position K400 to A, C, D, E, F, G, H, I, L, M, N, P, Q, R, S, T, V, W or Y, preferably to A, C, D, E, M, N, P, Q, S, T or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position W401 to A, C, D, E, F, G, H, I, K, L, M, N, P, Q, R, S, T, V or Y, preferably to F, H, K, L, M or R, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position F402 to A, C, D, E, G, H, I, K, L, M, N, P, Q, R, S, T, V, W or Y, preferably to T, W or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G403 to A, C, D, E, F, H, I, K, L, M, N, P, Q, R, S, T, V, W or Y, preferably to A, D, H, K, P, Q, S, T, V or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1. In another aspect, the variant comprises a substitution at position Q404 to A, C, D, E, F, G, H, I, K, L, M, N, P, R, S, T, V, W or Y, preferably to F, H, L, M, P, R, S or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1. In another aspect, the variant comprises a substitution at position A405 to C, D, E, F, G, H, I, K, L, M, N, P, Q, R, S, T, V, W or Y, preferably to C, E, H, K, P, R, T or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1. In another aspect, the variant comprises a substitution at position I406 to A, C, D, E, F, G, H, K, L, M, N, P, Q, R, S, T, V, W or Y, preferably to C, D or N, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A407 to C, D, E, F, G, H, I, K, L, M, N, P, Q, R, S, T, V, W or Y, preferably to C, G, M, Q, S, T or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G408 to A, C, D, E, F, H, I, K, L, M, N, P, Q, R, S, T, V, W or Y, preferably to D, I, M, N or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position D409 to A, C, E, F, G, H, I, K, L, M, N, P, Q, R, S, T, V, W or Y, preferably to N or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position N410 to A, C, D, E, F, G, H, I, K, L, M, P, Q, R, S, T, V, W or Y, preferably to C, R or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A411 to C, D, E, F, G, H, I, K, L, M, N, P, Q, R, S, T, V, W or Y, preferably to E, N, R, S or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V412 to A, C, D, E, F, G, H, I, K, L, M, N, P, Q, R, S, T, W or Y, preferably to M or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position L413 to A, C, D, E, F, G, H, I, K, M, N, P, Q, R, S, T, V, W or Y, preferably to D, E, F, I, P or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G414 to A, C, D, E, F, H, I, K, L, M, N, P, Q, R, S, T, V, W or Y, preferably to A, C, M, N, R, T or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G415 to A, C, D, E, F, H, I, K, L, M, N, P, Q, R, S, T, V, W or Y, preferably to A, Q or R, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position D416 to A, C, E, F, G, H, I, K, L, M, N, P, Q, R, S, T, V, W or Y, preferably to I, M, R, T or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1. In another aspect, the variant comprises a substitution at position K417 to A, C, D, E, F, G, H, I, L, M, N, P, Q, R, S, T, V, W or Y, preferably to C, F, G, R or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1. In another aspect, the variant comprises a substitution at position D418 to A, C, E, F, G, H, I, K, L, M, N, P, Q, R, S, T, V, W or Y, preferably to L, P, R or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position E419 to A, C, D, F, G, H, I, K, L, M, N, P, Q, R, S, T, V, W or Y, preferably to M, R or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T420 to A, C, D, E, F, G, H, I, K, L, M, N, P, Q, R, S, V, W or Y, preferably to E, F, G, K, R or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position W421 to A, C, D, E, F, G, H, I, K, L, M, N, P, Q, R, S, T, V or Y, preferably to L, Q or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A422 to C, D, E, F, G, H, I, K, L, M, N, P, Q, R, S, T, V, W or Y, preferably to P, T or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position K423 to A, C, D, E, F, G, H, I, L, M, N, P, Q, R, S, T, V, W or Y, preferably to D, L, M or R, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position F424 to A, C, D, E, G, H, I, K, L, M, N, P, Q, R, S, T, V, W or Y, preferably to C, L, N or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position D425 to A, C, E, F, G, H, I, K, L, M, N, P, Q, R, S, T, V, W or Y, preferably to E, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position L426 to A, C, D, E, F, G, H, I, K, M, N, P, Q, R, S, T, V, W or Y, preferably to C, M or Q, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T427 to A, C, D, E, F, G, H, I, K, L, M, N, P, Q, R, S, V, W or Y, preferably to D, F, G, K, M, P, Q, R, S or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1. In another aspect, the variant comprises a substitution at position S428 to A, C, D, E, F, G, H, I, K, L, M, N, P, Q, R, T, V, W or Y, preferably to F, K or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T429 to A, C, D, E, F, G, H, I, K, L, M, N, P, Q, R, S, V, W or Y, preferably to D or P, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position 1430 to A, C, D, E, F, G, H, K, L, M, N, P, Q, R, S, T, V, W or Y, preferably to C, D, E, L, M, Q, S, T or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position N431 to A, C, D, E, F, G, H, I, K, L, M, P, Q, R, S, T, V, W or Y, preferably to D, E, G, L, M, R, V or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position R432 to A, C, D, E, F, G, H, I, K, L, M, N, P, Q, S, T, V, W or Y, preferably to A, E, F, G, N, Q, V or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1. In another aspect, the variant comprises a substitution at position D433 to A, C, E, F, G, H, I, K, L, M, N, P, Q, R, S, T, V, W or Y, preferably to C, G, H, I, P, Q or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1. In another aspect, the variant comprises a substitution at position R434 to A, C, D, E, F, G, H, I, K, L, M, N, P, Q, S, T, V, W or Y, preferably to L, M, N, P, S, T or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1. In another aspect, the variant comprises a substitution at position N435 to A, C, D, E, F, G, H, I, K, L, M, P, Q, R, S, T, V, W or Y, preferably to E, F, H, K, L, M, R, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A436 to C, D, E, F, G, H, I, K, L, M, N, P, Q, R, S, T, V, W or Y, preferably to C, D, E, G, I, L, M, Q or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position P437 to A, C, D, E, F, G, H, I, K, L, M, N, Q, R, S, T, V, W or Y, preferably to A, D, K, L, Q, R, S, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position S438 to A, C, D, E, F, G, H, I, K, L, M, N, P, Q, R, T, V, W or Y, preferably to G, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V439 to A, C, D, E, F, G, H, I, K, L, M, N, P, Q, R, S, T, W or Y, preferably to C, E, G, I, K, Q, T or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1. In another aspect, the variant comprises a substitution at position 1440 to A, C, D, E, F, G, H, K, L, M, N, P, Q, R, S, T, V, W or Y, preferably to C, D, F, K, P, R, S, T, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1. In another aspect, the variant comprises a substitution at position M441 to A, C, D, E, F, G, H, I, K, L, N, P, Q, R, S, T, V, W or Y, preferably to A, E, G, Q, R, T or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position W442 to A, C, D, E, F, G, H, I, K, L, M, N, P, Q, R, S, T, V or Y, preferably to E, G, M, P, Q or R, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position S443 to A, C, D, E, F, G, H, I, K, L, M, N, P, Q, R, T, V, W or Y, preferably to C, D, G, M, Q or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position L444 to A, C, D, E, F, G, H, I, K, M, N, P, Q, R, S, T, V, W or Y, preferably to C, D, E, F, G, H, K, Q, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1. In another aspect, the variant comprises a substitution at position G445 to A, C, D, E, F, H, I, K, L, M, N, P, Q, R, S, T, V, W or Y, preferably to A, C or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position N446 to A, C, D, E, F, G, H, I, K, L, M, P, Q, R, S, T, V, W or Y, preferably to D or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position M448 to A, C, D, E, F, G, H, I, K, L, N, P, Q, R, S, T, V, W or Y, preferably to A, C, D, E, I, L, P, Q, S, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position M449 to A, C, D, E, F, G, H, I, K, L, N, P, Q, R, S, T, V, W or Y, preferably to D, E, F, T, V, W or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position E450 to A, C, D, F, G, H, I, K, L, M, N, P, Q, R, S, T, V, W or Y, preferably to F, S, T, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G451 to A, C, D, E, F, H, I, K, L, M, N, P, Q, R, S, T, V, W or Y, preferably to C, F, L, P, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1. In another aspect, the variant comprises a substitution at position 1452 to A, C, D, E, F, G, H, K, L, M, N, P, Q, R, S, T, V, W or Y, preferably to G, K, L, M, Q, S or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1. In another aspect, the variant comprises a substitution at position S453 to A, C, D, E, F, G, H, I, K, L, M, N, P, Q, R, T, V, W or Y, preferably to C, F, G, H, L, M, N, P, Q, R or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G454 to A, C, D, E, F, H, I, K, L, M, N, P, Q, R, S, T, V, W or Y, preferably to L or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position S455 to A, C, D, E, F, G, H, I, K, L, M, N, P, Q, R, T, V, W or Y, preferably to A, E, K, M, P, R, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V456 to A, C, D, E, F, G, H, I, K, L, M, N, P, Q, R, S, T, W or Y, preferably to A, D, E, F, K, L or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1. In another aspect, the variant comprises a substitution at position S457 to A, C, D, E, F, G, H, I, K, L, M, N, P, Q, R, T, V, W or Y, preferably to E, H, K, L, M, P, Q, T or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G458 to A, C, D, E, F, H, I, K, L, M, N, P, Q, R, S, T, V, W or Y, preferably to A, C, D, F, L, P, Q, S, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position F459 to A, C, D, E, G, H, I, K, L, M, N, P, Q, R, S, T, V, W or Y, preferably to A, C, E, G, N, R, S, T or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position P460 to A, C, D, E, F, G, H, I, K, L, M, N, Q, R, S, T, V, W or Y, preferably to C, M, Q, W or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A461 to C, D, E, F, G, H, I, K, L, M, N, P, Q, R, S, T, V, W or Y, preferably to D, G, M, N, Q, S, V or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T462 to A, C, D, E, F, G, H, I, K, L, M, N, P, Q, R, S, V, W or Y, preferably to C, E, F, L, M or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1. In another aspect, the variant comprises a substitution at position S463 to A, C, D, E, F, G, H, I, K, L, M, N, P, Q, R, T, V, W or Y, preferably to G, K, Q, R, T or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A464 to C, D, E, F, G, H, I, K, L, M, N, P, Q, R, S, T, V, W or Y, preferably to E, H, L, M, P, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position K465 to A, C, D, E, F, G, H, I, L, M, N, P, Q, R, S, T, V, W or Y, preferably to C, F, G, L, Q, R, V, W or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position L466 to A, C, D, E, F, G, H, I, K, M, N, P, Q, R, S, T, V, W or Y, preferably to A, C, D, E, F, G, M, P, Q, S, V or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V467 to A, C, D, E, F, G, H, I, K, L, M, N, P, Q, R, S, T, W or Y, preferably to A, C, D, E, G, T or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A468 to C, D, E, F, G, H, I, K, L, M, N, P, Q, R, S, T, V, W or Y, preferably to D, E, F, K, L, P, S, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position W469 to A, C, D, E, F, G, H, I, K, L, M, N, P, Q, R, S, T, V or Y, preferably to A, C, D, G, L, M, R, V or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T470 to A, C, D, E, F, G, H, I, K, L, M, N, P, Q, R, S, V, W or Y, preferably to E, L, M or Q, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position K471 to A, C, D, E, F, G, H, I, L, M, N, P, Q, R, S, T, V, W or Y, preferably to F, G, Q, W or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A472 to C, D, E, F, G, H, I, K, L, M, N, P, Q, R, S, T, V, W or Y, preferably to G or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A473 to C, D, E, F, G, H, I, K, L, M, N, P, Q, R, S, T, V, W or Y, preferably to E, M or P, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position D474 to A, C, E, F, G, H, I, K, L, M, N, P, Q, R, S, T, V, W or Y, preferably to A, C, E, K, M, R or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position S475 to A, C, D, E, F, G, H, I, K, L, M, N, P, Q, R, T, V, W or Y, preferably to E, F, Q, T or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T476 to A, C, D, E, F, G, H, I, K, L, M, N, P, Q, R, S, V, W or Y, preferably to C, L or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position R477 to A, C, D, E, F, G, H, I, K, L, M, N, P, Q, S, T, V, W or Y, preferably to A, C, G or L, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position P478 to A, C, D, E, F, G, H, I, K, L, M, N, Q, R, S, T, V, W or Y, preferably to A, D, G, L or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position M479 to A, C, D, E, F, G, H, I, K, L, N, P, Q, R, S, T, V, W or Y, preferably to G, I, R or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T480 to A, C, D, E, F, G, H, I, K, L, M, N, P, Q, R, S, V, W or Y, preferably to C, G or Q, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position K485 to A, C, D, E, F, G, H, I, L, M, N, P, Q, R, S, T, V, W or Y, preferably to E or R, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position K487 to A, C, D, E, F, G, H, I, L, M, N, P, Q, R, S, T, V, W or Y, preferably to A, C, F, G, N, S or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A488 to C, D, E, F, G, H, I, K, L, M, N, P, Q, R, S, T, V, W or Y, preferably to C, G, H, L, N, S or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position N491 to A, C, D, E, F, G, H, I, K, L, M, P, Q, R, S, T, V, W or Y, preferably to A, E or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position E492 to A, C, D, F, G, H, I, K, L, M, N, P, Q, R, S, T, V, W or Y, preferably to A or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position S493 to A, C, D, E, F, G, H, I, K, L, M, N, P, Q, R, T, V, W or Y, preferably to E, G, H, L, M, Q or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position N494 to A, C, D, E, F, G, H, I, K, L, M, P, Q, R, S, T, V, W or Y, preferably to A, I, M, R or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T495 to A, C, D, E, F, G, H, I, K, L, M, N, P, Q, R, S, V, W or Y, preferably to K, R, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position M496 to A, C, D, E, F, G, H, I, K, L, N, P, Q, R, S, T, V, W or Y, preferably to A, F or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G497 to A, C, D, E, F, H, I, K, L, M, N, P, Q, R, S, T, V, W or Y, preferably to D, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position D498 to A, C, E, F, G, H, I, K, L, M, N, P, Q, R, S, T, V, W or Y, preferably to A, C, M or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position N499 to A, C, D, E, F, G, H, I, K, L, M, P, Q, R, S, T, V, W or Y, preferably to K, R, T or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position L500 to A, C, D, E, F, G, H, I, K, M, N, P, Q, R, S, T, V, W or Y, preferably to A, E, N or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T501 to A, C, D, E, F, G, H, I, K, L, M, N, P, Q, R, S, V, W or Y, preferably to C, G or M, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A502 to C, D, E, F, G, H, I, K, L, M, N, P, Q, R, S, T, V, W or Y, preferably to L or Q, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position N503 to A, C, D, E, F, G, H, I, K, L, M, P, Q, R, S, T, V, W or Y, preferably to A, E, M or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G504 to A, C, D, E, F, H, I, K, L, M, N, P, Q, R, S, T, V, W or Y, preferably to H, K or P, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G505 to A, C, D, E, F, H, I, K, L, M, N, P, Q, R, S, T, V, W or Y, preferably to A, D, E, H, L, N, R, S or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V506 to A, C, D, E, F, G, H, I, K, L, M, N, P, Q, R, S, T, W or Y, preferably to C, D, E, G, I, L, P, R, S, T or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V507 to A, C, D, E, F, G, H, I, K, L, M, N, P, Q, R, S, T, W or Y, preferably to A, F, G, L, N, P, R, S or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G508 to A, C, D, E, F, H, I, K, L, M, N, P, Q, R, S, T, V, W or Y, preferably to C or E, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T509 to A, C, D, E, F, G, H, I, K, L, M, N, P, Q, R, S, V, W or Y, preferably to A, D, E, I, K, M, Q, S, V or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position N510 to A, C, D, E, F, G, H, I, K, L, M, P, Q, R, S, T, V, W or Y, preferably to A, F, I or Q, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position Y511 to A, C, D, E, F, G, H, I, K, L, M, N, P, Q, R, S, T, V or W, preferably to A or K, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position S512 to A, C, D, E, F, G, H, I, K, L, M, N, P, Q, R, T, V, W or Y, preferably to C, E, F, G, I, M, Q, T, V or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position D513 to A, C, E, F, G, H, I, K, L, M, N, P, Q, R, S, T, V, W or Y, preferably to C, G, K, L, M, P, Q, R or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G514 to A, C, D, E, F, H, I, K, L, M, N, P, Q, R, S, T, V, W or Y, preferably to F, L, N, P or R, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A515 to C, D, E, F, G, H, I, K, L, M, N, P, Q, R, S, T, V, W or Y, preferably to C, D, E, F, G, K, P, R or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position N516 to A, C, D, E, F, G, H, I, K, L, M, P, Q, R, S, T, V, W or Y, preferably to C, E, G, I, M, Q, S, T or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position Y517 to A, C, D, E, F, G, H, I, K, L, M, N, P, Q, R, S, T, V or W, preferably to G, I or N, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position D518 to A, C, E, F, G, H, I, K, L, M, N, P, Q, R, S, T, V, W or Y, preferably to Q or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position K519 to A, C, D, E, F, G, H, I, L, M, N, P, Q, R, S, T, V, W or Y, preferably to C, E, F, G, I, L, M, N, Q, S or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position 1520 to A, C, D, E, F, G, H, K, L, M, N, P, Q, R, S, T, V, W or Y, preferably to A, C, G, H, M, N, Q, S, T, W or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position R521 to A, C, D, E, F, G, H, I, K, L, M, N, P, Q, S, T, V, W or Y, preferably to A, C, K, N, Q or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T522 to A, C, D, E, F, G, H, I, K, L, M, N, P, Q, R, S, V, W or Y, preferably to G, I or N, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T523 to A, C, D, E, F, G, H, I, K, L, M, N, P, Q, R, S, V, W or Y, preferably to A or M, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position H524 to A, C, D, E, F, G, I, K, L, M, N, P, Q, R, S, T, V, W or Y, preferably to R or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position P525 to A, C, D, E, F, G, H, I, K, L, M, N, Q, R, S, T, V, W or Y, preferably to D, G, R, T or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position S526 to A, C, D, E, F, G, H, I, K, L, M, N, P, Q, R, T, V, W or Y, preferably to G, I or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position W527 to A, C, D, E, F, G, H, I, K, L, M, N, P, Q, R, S, T, V or Y, preferably to A, E, G, H, N, R, S or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A528 to C, D, E, F, G, H, I, K, L, M, N, P, Q, R, S, T, V, W or Y, preferably to C, E, G, I or L, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position 1529 to A, C, D, E, F, G, H, K, L, M, N, P, Q, R, S, T, V, W or Y, preferably to F, G, L or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position Y530 to A, C, D, E, F, G, H, I, K, L, M, N, P, Q, R, S, T, V or W, preferably to A or M, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G531 to A, C, D, E, F, H, I, K, L, M, N, P, Q, R, S, T, V, W or Y, preferably to E, S or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T534 to A, C, D, E, F, G, H, I, K, L, M, N, P, Q, R, S, V, W or Y, preferably to A, I or Q, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A535 to C, D, E, F, G, H, I, K, L, M, N, P, Q, R, S, T, V, W or Y, preferably to E, G, I or M, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A537 to C, D, E, F, G, H, I, K, L, M, N, P, Q, R, S, T, V, W or Y, preferably to D, M, P or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position 1538 to A, C, D, E, F, G, H, K, L, M, N, P, Q, R, S, T, V, W or Y, preferably to H or M, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position N539 to A, C, D, E, F, G, H, I, K, L, M, P, Q, R, S, T, V, W or Y, preferably to G or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position S540 to A, C, D, E, F, G, H, I, K, L, M, N, P, Q, R, T, V, W or Y, preferably to E, G or M, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G542 to A, C, D, E, F, H, I, K, L, M, N, P, Q, R, S, T, V, W or Y, preferably to E, Q, S or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position 1543 to A, C, D, E, F, G, H, K, L, M, N, P, Q, R, S, T, V, W or Y, preferably to V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position N545 to A, C, D, E, F, G, H, I, K, L, M, P, Q, R, S, T, V, W or Y, preferably to G, I, Q or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position R546 to A, C, D, E, F, G, H, I, K, L, M, N, P, Q, S, T, V, W or Y, preferably to A, C, L, P or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T547 to A, C, D, E, F, G, H, I, K, L, M, N, P, Q, R, S, V, W or Y, preferably to A, D, H, K, N or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T548 to A, C, D, E, F, G, H, I, K, L, M, N, P, Q, R, S, V, W or Y, preferably to D, E, F, K, L, P or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G549 to A, C, D, E, F, H, I, K, L, M, N, P, Q, R, S, T, V, W or Y, preferably to D, F, P or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G550 to A, C, D, E, F, H, I, K, L, M, N, P, Q, R, S, T, V, W or Y, preferably to Q, R or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A551 to C, D, E, F, G, H, I, K, L, M, N, P, Q, R, S, T, V, W or Y, preferably to D, I or Q, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position S553 to A, C, D, E, F, G, H, I, K, L, M, N, P, Q, R, T, V, W or Y, preferably to C, F, N, P, R, T or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position S554 to A, C, D, E, F, G, H, I, K, L, M, N, P, Q, R, T, V, W or Y, preferably to F, N, R, T, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position D555 to A, C, E, F, G, H, I, K, L, M, N, P, Q, R, S, T, V, W or Y, preferably to E, P or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position K556 to A, C, D, E, F, G, H, I, L, M, N, P, Q, R, S, T, V, W or Y, preferably to A, C, R or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position Q557 to A, C, D, E, F, G, H, I, K, L, M, N, P, R, S, T, V, W or Y, preferably to F, R or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position L558 to A, C, D, E, F, G, H, I, K, M, N, P, Q, R, S, T, V, W or Y, preferably to E, H, I or P, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T559 to A, C, D, E, F, G, H, I, K, L, M, N, P, Q, R, S, V, W or Y, preferably to A, G, I, Q, V or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position S560 to A, C, D, E, F, G, H, I, K, L, M, N, P, Q, R, T, V, W or Y, preferably to P or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position Y561 to A, C, D, E, F, G, H, I, K, L, M, N, P, Q, R, S, T, V or W, preferably to R, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position N563 to A, C, D, E, F, G, H, I, K, L, M, P, Q, R, S, T, V, W or Y, preferably to R or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position S564 to A, C, D, E, F, G, H, I, K, L, M, N, P, Q, R, T, V, W or Y, preferably to A, C, F or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A565 to C, D, E, F, G, H, I, K, L, M, N, P, Q, R, S, T, V, W or Y, preferably to C, D, E, F, G, I, L, M, R, S, T, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G567 to A, C, D, E, F, H, I, K, L, M, N, P, Q, R, S, T, V, W or Y, preferably to N, Q or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A570 to C, D, E, F, G, H, I, K, L, M, N, P, Q, R, S, T, V, W or Y, preferably to G, K, L, M, S, T or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V571 to A, C, D, E, F, G, H, I, K, L, M, N, P, Q, R, S, T, W or Y, preferably to W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A572 to C, D, E, F, G, H, I, K, L, M, N, P, Q, R, S, T, V, W or Y, preferably to S or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position S573 to A, C, D, E, F, G, H, I, K, L, M, N, P, Q, R, T, V, W or Y, preferably to G, K or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position S574 to A, C, D, E, F, G, H, I, K, L, M, N, P, Q, R, T, V, W or Y, preferably to K, Q, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A575 to C, D, E, F, G, H, I, K, L, M, N, P, Q, R, S, T, V, W or Y, preferably to D, M or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position W576 to A, C, D, E, F, G, H, I, K, L, M, N, P, Q, R, S, T, V or Y, preferably to A, F, V or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position Y577 to A, C, D, E, F, G, H, I, K, L, M, N, P, Q, R, S, T, V or W, preferably to G, I, L or R, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position D578 to A, C, E, F, G, H, I, K, L, M, N, P, Q, R, S, T, V, W or Y, preferably to E, M, N or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V579 to A, C, D, E, F, G, H, I, K, L, M, N, P, Q, R, S, T, W or Y, preferably to E, G, L or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V580 to A, C, D, E, F, G, H, I, K, L, M, N, P, Q, R, S, T, W or Y, preferably to A, D, E, K, L or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position Q581 to A, C, D, E, F, G, H, I, K, L, M, N, P, R, S, T, V, W or Y, preferably to F, G, P, R, S, T or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position R582 to A, C, D, E, F, G, H, I, K, L, M, N, P, Q, S, T, V, W or Y, preferably to A, D, G, I, L or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position D583 to A, C, E, F, G, H, I, K, L, M, N, P, Q, R, S, T, V, W or Y, preferably to V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position F584 to A, C, D, E, G, H, I, K, L, M, N, P, Q, R, S, T, V, W or Y, preferably to E, I or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V585 to A, C, D, E, F, G, H, I, K, L, M, N, P, Q, R, S, T, W or Y, preferably to I, M or Q, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A586 to C, D, E, F, G, H, I, K, L, M, N, P, Q, R, S, T, V, W or Y, preferably to C, D, H or K, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G587 to A, C, D, E, F, H, I, K, L, M, N, P, Q, R, S, T, V, W or Y, preferably to A or C, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T588 to A, C, D, E, F, G, H, I, K, L, M, N, P, Q, R, S, V, W or Y, preferably to C, D, G, I, L, M or P, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position Y589 to A, C, D, E, F, G, H, I, K, L, M, N, P, Q, R, S, T, V or W, preferably to A, I, Q, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V590 to A, C, D, E, F, G, H, I, K, L, M, N, P, Q, R, S, T, W or Y, preferably to A, H or I, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position W591 to A, C, D, E, F, G, H, I, K, L, M, N, P, Q, R, S, T, V or Y, preferably to F, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T592 to A, C, D, E, F, G, H, I, K, L, M, N, P, Q, R, S, V, W or Y, preferably to C, L, Q or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G593 to A, C, D, E, F, H, I, K, L, M, N, P, Q, R, S, T, V, W or Y, preferably to C or I, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position F594 to A, C, D, E, G, H, I, K, L, M, N, P, Q, R, S, T, V, W or Y, preferably to C, I, L or M, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position D595 to A, C, E, F, G, H, I, K, L, M, N, P, Q, R, S, T, V, W or Y, preferably to E, Q or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position L597 to A, C, D, E, F, G, H, I, K, M, N, P, Q, R, S, T, V, W or Y, preferably to C, D, E or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G598 to A, C, D, E, F, H, I, K, L, M, N, P, Q, R, S, T, V, W or Y, preferably to N, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position P600 to A, C, D, E, F, G, H, I, K, L, M, N, Q, R, S, T, V, W or Y, preferably to A, E, G or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position N604 to A, C, D, E, F, G, H, I, K, L, M, P, Q, R, S, T, V, W or Y, preferably to E or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G605 to A, C, D, E, F, H, I, K, L, M, N, P, Q, R, S, T, V, W or Y, preferably to R, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T606 to A, C, D, E, F, G, H, I, K, L, M, N, P, Q, R, S, V, W or Y, preferably to S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G607 to A, C, D, E, F, H, I, K, L, M, N, P, Q, R, S, T, V, W or Y, preferably to A, Q or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position S608 to A, C, D, E, F, G, H, I, K, L, M, N, P, Q, R, T, V, W or Y, preferably to E, I, M, N, Q, T or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G609 to A, C, D, E, F, H, I, K, L, M, N, P, Q, R, S, T, V, W or Y, preferably to A, H, L, N, R or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A610 to C, D, E, F, G, H, I, K, L, M, N, P, Q, R, S, T, V, W or Y, preferably to D, F, M or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V611 to A, C, D, E, F, G, H, I, K, L, M, N, P, Q, R, S, T, W or Y, preferably to K, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G612 to A, C, D, E, F, H, I, K, L, M, N, P, Q, R, S, T, V, W or Y, preferably to N, T or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position S613 to A, C, D, E, F, G, H, I, K, L, M, N, P, Q, R, T, V, W or Y, preferably to A, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position W614 to A, C, D, E, F, G, H, I, K, L, M, N, P, Q, R, S, T, V or Y, preferably to A, F or P, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position P615 to A, C, D, E, F, G, H, I, K, L, M, N, Q, R, S, T, V, W or Y, preferably to L, S or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position S616 to A, C, D, E, F, G, H, I, K, L, M, N, P, Q, R, T, V, W or Y, preferably to A or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position N619 to A, C, D, E, F, G, H, I, K, L, M, P, Q, R, S, T, V, W or Y, preferably to A, I, L or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position S620 to A, C, D, E, F, G, H, I, K, L, M, N, P, Q, R, T, V, W or Y, preferably to G, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position Y621 to A, C, D, E, F, G, H, I, K, L, M, N, P, Q, R, S, T, V or W, preferably to W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position 1624 to A, C, D, E, F, G, H, K, L, M, N, P, Q, R, S, T, V, W or Y, preferably to A or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V625 to A, C, D, E, F, G, H, I, K, L, M, N, P, Q, R, S, T, W or Y, preferably to A, E, F, M or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T627 to A, C, D, E, F, G, H, I, K, L, M, N, P, Q, R, S, V, W or Y, preferably to F, G, K or Q, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A628 to C, D, E, F, G, H, I, K, L, M, N, P, Q, R, S, T, V, W or Y, preferably to C, D or N, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G629 to A, C, D, E, F, H, I, K, L, M, N, P, Q, R, S, T, V, W or Y, preferably to T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position F630 to A, C, D, E, G, H, I, K, L, M, N, P, Q, R, S, T, V, W or Y, preferably to A, C, D, G, S or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position P631 to A, C, D, E, F, G, H, I, K, L, M, N, Q, R, S, T, V, W or Y, preferably to A, D, G, H, S, V or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position K632 to A, C, D, E, F, G, H, I, L, M, N, P, Q, R, S, T, V, W or Y, preferably to C, D, G or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position D633 to A, C, E, F, G, H, I, K, L, M, N, P, Q, R, S, T, V, W or Y, preferably to G, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T634 to A, C, D, E, F, G, H, I, K, L, M, N, P, Q, R, S, V, W or Y, preferably to A, E, F, S or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position Y635 to A, C, D, E, F, G, H, I, K, L, M, N, P, Q, R, S, T, V or W, preferably to R, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position Y636 to A, C, D, E, F, G, H, I, K, L, M, N, P, Q, R, S, T, V or W, preferably to K, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position F637 to A, C, D, E, G, H, I, K, L, M, N, P, Q, R, S, T, V, W or Y, preferably to C, G, I, S, T or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position Y638 to A, C, D, E, F, G, H, I, K, L, M, N, P, Q, R, S, T, V or W, preferably to A or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position Q639 to A, C, D, E, F, G, H, I, K, L, M, N, P, R, S, T, V, W or Y, preferably to D, N or R, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position S640 to A, C, D, E, F, G, H, I, K, L, M, N, P, Q, R, T, V, W or Y, preferably to C or D, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position Q641 to A, C, D, E, F, G, H, I, K, L, M, N, P, R, S, T, V, W or Y, preferably to T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position W642 to A, C, D, E, F, G, H, I, K, L, M, N, P, Q, R, S, T, V or Y, preferably to I, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position N643 to A, C, D, E, F, G, H, I, K, L, M, P, Q, R, S, T, V, W or Y, preferably to R, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position D644 to A, C, E, F, G, H, I, K, L, M, N, P, Q, R, S, T, V, W or Y, preferably to C, G or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position D645 to A, C, E, F, G, H, I, K, L, M, N, P, Q, R, S, T, V, W or Y, preferably to S or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V646 to A, C, D, E, F, G, H, I, K, L, M, N, P, Q, R, S, T, W or Y, preferably to C, L, N, R or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position H647 to A, C, D, E, F, G, I, K, L, M, N, P, Q, R, S, T, V, W or Y, preferably to G or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T648 to A, C, D, E, F, G, H, I, K, L, M, N, P, Q, R, S, V, W or Y, preferably to C, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position L649 to A, C, D, E, F, G, H, I, K, M, N, P, Q, R, S, T, V, W or Y, preferably to V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position H650 to A, C, D, E, F, G, I, K, L, M, N, P, Q, R, S, T, V, W or Y, preferably to E, F or R, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position 1651 to A, C, D, E, F, G, H, K, L, M, N, P, Q, R, S, T, V, W or Y, preferably to F, T or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position L652 to A, C, D, E, F, G, H, I, K, M, N, P, Q, R, S, T, V, W or Y, preferably to C, D, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position P653 to A, C, D, E, F, G, H, I, K, L, M, N, Q, R, S, T, V, W or Y, preferably to Q, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A654 to C, D, E, F, G, H, I, K, L, M, N, P, Q, R, S, T, V, W or Y, preferably to D, K, M or R, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position W655 to A, C, D, E, F, G, H, I, K, L, M, N, P, Q, R, S, T, V or Y, preferably to F or R, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position N656 to A, C, D, E, F, G, H, I, K, L, M, P, Q, R, S, T, V, W or Y, preferably to A, K or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position E657 to A, C, D, F, G, H, I, K, L, M, N, P, Q, R, S, T, V, W or Y, preferably to K, R or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V659 to A, C, D, E, F, G, H, I, K, L, M, N, P, Q, R, S, T, W or Y, preferably to D or N, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A661 to C, D, E, F, G, H, I, K, L, M, N, P, Q, R, S, T, V, W or Y, preferably to E, G, H, K, L, M, Q or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position K662 to A, C, D, E, F, G, H, I, L, M, N, P, Q, R, S, T, V, W or Y, preferably to H, S, V, W or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position N667 to A, C, D, E, F, G, H, I, K, L, M, P, Q, R, S, T, V, W or Y, preferably to L or R, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position P669 to A, C, D, E, F, G, H, I, K, L, M, N, Q, R, S, T, V, W or Y, preferably to A, E, F, L, R, T or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V670 to A, C, D, E, F, G, H, I, K, L, M, N, P, Q, R, S, T, W or Y, preferably to C, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V672 to A, C, D, E, F, G, H, I, K, L, M, N, P, Q, R, S, T, W or Y, preferably to L, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position Y673 to A, C, D, E, F, G, H, I, K, L, M, N, P, Q, R, S, T, V or W, preferably to E, G, I, R, S or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T674 to A, C, D, E, F, G, H, I, K, L, M, N, P, Q, R, S, V, W or Y, preferably to C, D, G, H, M or Q, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position D675 to A, C, E, F, G, H, I, K, L, M, N, P, Q, R, S, T, V, W or Y, preferably to A, E, P, Q, S, V or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A676 to C, D, E, F, G, H, I, K, L, M, N, P, Q, R, S, T, V, W or Y, preferably to C, E, G, K, L, P, S or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A677 to C, D, E, F, G, H, I, K, L, M, N, P, Q, R, S, T, V, W or Y, preferably to E, G, L, R, T, V or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position K678 to A, C, D, E, F, G, H, I, L, M, N, P, Q, R, S, T, V, W or Y, preferably to A, C, T or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V679 to A, C, D, E, F, G, H, I, K, L, M, N, P, Q, R, S, T, W or Y, preferably to G, Q, S, T or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position K680 to A, C, D, E, F, G, H, I, L, M, N, P, Q, R, S, T, V, W or Y, preferably to A, E, G, H, I, L, N, Q, S, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position L681 to A, C, D, E, F, G, H, I, K, M, N, P, Q, R, S, T, V, W or Y, preferably to E, F, G, M, S or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position Y682 to A, C, D, E, F, G, H, I, K, L, M, N, P, Q, R, S, T, V or W, preferably to D, E, I, M, S, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position F683 to A, C, D, E, G, H, I, K, L, M, N, P, Q, R, S, T, V, W or Y, preferably to H, L, M, Q, R or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T684 to A, C, D, E, F, G, H, I, K, L, M, N, P, Q, R, S, V, W or Y, preferably to A, D, G, L, R, S or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position P685 to A, C, D, E, F, G, H, I, K, L, M, N, Q, R, S, T, V, W or Y, preferably to A, E, I, L or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position K686 to A, C, D, E, F, G, H, I, L, M, N, P, Q, R, S, T, V, W or Y, preferably to A, E, I, M, T or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G687 to A, C, D, E, F, H, I, K, L, M, N, P, Q, R, S, T, V, W or Y, preferably to A, K, N, P, Q or R, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position S688 to A, C, D, E, F, G, H, I, K, L, M, N, P, Q, R, T, V, W or Y, preferably to C, D, E, G, K, L, P or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T689 to A, C, D, E, F, G, H, I, K, L, M, N, P, Q, R, S, V, W or Y, preferably to A, D, E, G, L, P, S, W or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position E690 to A, C, D, F, G, H, I, K, L, M, N, P, Q, R, S, T, V, W or Y, preferably to D, M, P, T or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position K691 to A, C, D, E, F, G, H, I, L, M, N, P, Q, R, S, T, V, W or Y, preferably to E, H, N, P, R or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position R692 to A, C, D, E, F, G, H, I, K, L, M, N, P, Q, S, T, V, W or Y, preferably to G, H, I, L, P, S, T, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position L693 to A, C, D, E, F, G, H, I, K, M, N, P, Q, R, S, T, V, W or Y, preferably to A, M or P, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position 1694 to A, C, D, E, F, G, H, K, L, M, N, P, Q, R, S, T, V, W or Y, preferably to L or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G695 to A, C, D, E, F, H, I, K, L, M, N, P, Q, R, S, T, V, W or Y, preferably to C, K, L, R or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position E696 to A, C, D, F, G, H, I, K, L, M, N, P, Q, R, S, T, V, W or Y, preferably to A, L or R, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position K697 to A, C, D, E, F, G, H, I, L, M, N, P, Q, R, S, T, V, W or Y, preferably to A, E, G, R, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position S698 to A, C, D, E, F, G, H, I, K, L, M, N, P, Q, R, T, V, W or Y, preferably to C, D, E, I, L, M, P, Q, R or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T700 to A, C, D, E, F, G, H, I, K, L, M, N, P, Q, R, S, V, W or Y, preferably to A, C, D, E, G, K or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position K701 to A, C, D, E, F, G, H, I, L, M, N, P, Q, R, S, T, V, W or Y, preferably to A, D, E, G, H, L, M, P, S or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T703 to A, C, D, E, F, G, H, I, K, L, M, N, P, Q, R, S, V, W or Y, preferably to E, I, K or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T704 to A, C, D, E, F, G, H, I, K, L, M, N, P, Q, R, S, V, W or Y, preferably to M, R or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO:

In another aspect, the variant comprises a substitution at position A705 to C, D, E, F, G, H, I, K, L, M, N, P, Q, R, S, T, V, W or Y, preferably to C, D, E, K, N, P, R, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A706 to C, D, E, F, G, H, I, K, L, M, N, P, Q, R, S, T, V, W or Y, preferably to C, G, T, W or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position Y708 to A, C, D, E, F, G, H, I, K, L, M, N, P, Q, R, S, T, V or W, preferably to C, F, G, K, L, P or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T709 to A, C, D, E, F, G, H, I, K, L, M, N, P, Q, R, S, V, W or Y, preferably to M, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position Y710 to A, C, D, E, F, G, H, I, K, L, M, N, P, Q, R, S, T, V or W, preferably to C, D, E, G, M, N, T, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position Q711 to A, C, D, E, F, G, H, I, K, L, M, N, P, R, S, T, V, W or Y, preferably to A, D, G, L, M, T or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V712 to A, C, D, E, F, G, H, I, K, L, M, N, P, Q, R, S, T, W or Y, preferably to G, M, P, Q or R, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position Y713 to A, C, D, E, F, G, H, I, K, L, M, N, P, Q, R, S, T, V or W, preferably to A, E, G, L or Q, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position E714 to A, C, D, F, G, H, I, K, L, M, N, P, Q, R, S, T, V, W or Y, preferably to D, H, I, K, M, N, R, S, V or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G715 to A, C, D, E, F, H, I, K, L, M, N, P, Q, R, S, T, V, W or Y, preferably to K, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A716 to C, D, E, F, G, H, I, K, L, M, N, P, Q, R, S, T, V, W or Y, preferably to C, G, L, M, P, R, T or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position D717 to A, C, E, F, G, H, I, K, L, M, N, P, Q, R, S, T, V, W or Y, preferably to C, G, L or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position K718 to A, C, D, E, F, G, H, I, L, M, N, P, Q, R, S, T, V, W or Y, preferably to A, L, Q, T, W or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position D719 to A, C, E, F, G, H, I, K, L, M, N, P, Q, R, S, T, V, W or Y, preferably to E, L, P, S, T or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position S720 to A, C, D, E, F, G, H, I, K, L, M, N, P, Q, R, T, V, W or Y, preferably to A, M, R or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T721 to A, C, D, E, F, G, H, I, K, L, M, N, P, Q, R, S, V, W or Y, preferably to D, H, K, L, N, P, Q, S, V, W or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A722 to C, D, E, F, G, H, I, K, L, M, N, P, Q, R, S, T, V, W or Y, preferably to Q, T or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO:

In another aspect, the variant comprises a substitution at position H723 to A, C, D, E, F, G, I, K, L, M, N, P, Q, R, S, T, V, W or Y, preferably to G or P, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position K724 to A, C, D, E, F, G, H, I, L, M, N, P, Q, R, S, T, V, W or Y, preferably to A, G or R, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position N725 to A, C, D, E, F, G, H, I, K, L, M, P, Q, R, S, T, V, W or Y, preferably to A, D or I, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position M726 to A, C, D, E, F, G, H, I, K, L, N, P, Q, R, S, T, V, W or Y, preferably to A, D, E, K, Q, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position Y727 to A, C, D, E, F, G, H, I, K, L, M, N, P, Q, R, S, T, V or W, preferably to L or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position L728 to A, C, D, E, F, G, H, I, K, M, N, P, Q, R, S, T, V, W or Y, preferably to C, K, Q, S, T or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T729 to A, C, D, E, F, G, H, I, K, L, M, N, P, Q, R, S, V, W or Y, preferably to A, E or M, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position W730 to A, C, D, E, F, G, H, I, K, L, M, N, P, Q, R, S, T, V or Y, preferably to A, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position N731 to A, C, D, E, F, G, H, I, K, L, M, P, Q, R, S, T, V, W or Y, preferably to A, E, Q, S or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V732 to A, C, D, E, F, G, H, I, K, L, M, N, P, Q, R, S, T, W or Y, preferably to G, P, R or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position P733 to A, C, D, E, F, G, H, I, K, L, M, N, Q, R, S, T, V, W or Y, preferably to A, R or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position W734 to A, C, D, E, F, G, H, I, K, L, M, N, P, Q, R, S, T, V or Y, preferably to D, G, R or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A735 to C, D, E, F, G, H, I, K, L, M, N, P, Q, R, S, T, V, W or Y, preferably to Q, R, S, T or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position E736 to A, C, D, F, G, H, I, K, L, M, N, P, Q, R, S, T, V, W or Y, preferably to S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G737 to A, C, D, E, F, H, I, K, L, M, N, P, Q, R, S, T, V, W or Y, preferably to F, K, N or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T738 to A, C, D, E, F, G, H, I, K, L, M, N, P, Q, R, S, V, W or Y, preferably to G, L or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position 1739 to A, C, D, E, F, G, H, K, L, M, N, P, Q, R, S, T, V, W or Y, preferably to E, K, M, W or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position S740 to A, C, D, E, F, G, H, I, K, L, M, N, P, Q, R, T, V, W or Y, preferably to D, E, F, H or L, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A741 to C, D, E, F, G, H, I, K, L, M, N, P, Q, R, S, T, V, W or Y, preferably to I, P, S or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position E742 to A, C, D, F, G, H, I, K, L, M, N, P, Q, R, S, T, V, W or Y, preferably to D, M, Q or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A743 to C, D, E, F, G, H, I, K, L, M, N, P, Q, R, S, T, V, W or Y, preferably to C, E, H, I or L, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position Y744 to A, C, D, E, F, G, H, I, K, L, M, N, P, Q, R, S, T, V or W, preferably to A, E, I, K, L or R, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position D745 to A, C, E, F, G, H, I, K, L, M, N, P, Q, R, S, T, V, W or Y, preferably to C, F, N or R, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position E746 to A, C, D, F, G, H, I, K, L, M, N, P, Q, R, S, T, V, W or Y, preferably to A, C, K or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position N747 to A, C, D, E, F, G, H, I, K, L, M, P, Q, R, S, T, V, W or Y, preferably to E, F, G, P or R, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position N748 to A, C, D, E, F, G, H, I, K, L, M, P, Q, R, S, T, V, W or Y, preferably to S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position R749 to A, C, D, E, F, G, H, I, K, L, M, N, P, Q, S, T, V, W or Y, preferably to H, M, S or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position L750 to A, C, D, E, F, G, H, I, K, M, N, P, Q, R, S, T, V, W or Y, preferably to G, M, P, Q or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position 1751 to A, C, D, E, F, G, H, K, L, M, N, P, Q, R, S, T, V, W or Y, preferably to C, H, Q or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position P752 to A, C, D, E, F, G, H, I, K, L, M, N, Q, R, S, T, V, W or Y, preferably to A, C, H, L, S, V or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position E753 to A, C, D, F, G, H, I, K, L, M, N, P, Q, R, S, T, V, W or Y, preferably to Q, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G754 to A, C, D, E, F, H, I, K, L, M, N, P, Q, R, S, T, V, W or Y, preferably to H, I, P or R, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position S755 to A, C, D, E, F, G, H, I, K, L, M, N, P, Q, R, T, V, W or Y, preferably to C, I or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T756 to A, C, D, E, F, G, H, I, K, L, M, N, P, Q, R, S, V, W or Y, preferably to E, N, P, Q, S or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position E757 to A, C, D, F, G, H, I, K, L, M, N, P, Q, R, S, T, V, W or Y, preferably to A, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G758 to A, C, D, E, F, H, I, K, L, M, N, P, Q, R, S, T, V, W or Y, preferably to A or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position N759 to A, C, D, E, F, G, H, I, K, L, M, P, Q, R, S, T, V, W or Y, preferably to D, R, S or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A760 to C, D, E, F, G, H, I, K, L, M, N, P, Q, R, S, T, V, W or Y, preferably to G, N, P or Q, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position S761 to A, C, D, E, F, G, H, I, K, L, M, N, P, Q, R, T, V, W or Y, preferably to A, K or Q, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V762 to A, C, D, E, F, G, H, I, K, L, M, N, P, Q, R, S, T, W or Y, preferably to D, G, K or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T765 to A, C, D, E, F, G, H, I, K, L, M, N, P, Q, R, S, V, W or Y, preferably to A, P, R or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G766 to A, C, D, E, F, H, I, K, L, M, N, P, Q, R, S, T, V, W or Y, preferably to M or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A768 to C, D, E, F, G, H, I, K, L, M, N, P, Q, R, S, T, V, W or Y, preferably to H, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A769 to C, D, E, F, G, H, I, K, L, M, N, P, Q, R, S, T, V, W or Y, preferably to F, G, I, N, V or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position K770 to A, C, D, E, F, G, H, I, L, M, N, P, Q, R, S, T, V, W or Y, preferably to H, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position L771 to A, C, D, E, F, G, H, I, K, M, N, P, Q, R, S, T, V, W or Y, preferably to A or I, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position K772 to A, C, D, E, F, G, H, I, L, M, N, P, Q, R, S, T, V, W or Y, preferably to A, C, P, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A773 to C, D, E, F, G, H, I, K, L, M, N, P, Q, R, S, T, V, W or Y, preferably to C, G, H, M, R, S or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position D774 to A, C, E, F, G, H, I, K, L, M, N, P, Q, R, S, T, V, W or Y, preferably to A or R, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A775 to C, D, E, F, G, H, I, K, L, M, N, P, Q, R, S, T, V, W or Y, preferably to L, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position D776 to A, C, E, F, G, H, I, K, L, M, N, P, Q, R, S, T, V, W or Y, preferably to I, L, R, S or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position R777 to A, C, D, E, F, G, H, I, K, L, M, N, P, Q, S, T, V, W or Y, preferably to D, E, G, H, P, S or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position K778 to A, C, D, E, F, G, H, I, L, M, N, P, Q, R, S, T, V, W or Y, preferably to A, C, F, G, L, N or R, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T779 to A, C, D, E, F, G, H, I, K, L, M, N, P, Q, R, S, V, W or Y, preferably to C or I, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position 1780 to A, C, D, E, F, G, H, K, L, M, N, P, Q, R, S, T, V, W or Y, preferably to V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T781 to A, C, D, E, F, G, H, I, K, L, M, N, P, Q, R, S, V, W or Y, preferably to A, C, E, F, G, M, P, R or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A782 to C, D, E, F, G, H, I, K, L, M, N, P, Q, R, S, T, V, W or Y, preferably to C, E, K, N, P, Q or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position D783 to A, C, E, F, G, H, I, K, L, M, N, P, Q, R, S, T, V, W or Y, preferably to A, C, E or R, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G784 to A, C, D, E, F, H, I, K, L, M, N, P, Q, R, S, T, V, W or Y, preferably to A, F, L, S or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position K785 to A, C, D, E, F, G, H, I, L, M, N, P, Q, R, S, T, V, W or Y, preferably to C, I, S, V, W or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position D786 to A, C, E, F, G, H, I, K, L, M, N, P, Q, R, S, T, V, W or Y, preferably to S or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position L787 to A, C, D, E, F, G, H, I, K, M, N, P, Q, R, S, T, V, W or Y, preferably to D, K, P, T or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position S788 to A, C, D, E, F, G, H, I, K, L, M, N, P, Q, R, T, V, W or Y, preferably to A, G or I, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position Y789 to A, C, D, E, F, G, H, I, K, L, M, N, P, Q, R, S, T, V or W, preferably to C, D, I or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position 1790 to A, C, D, E, F, G, H, K, L, M, N, P, Q, R, S, T, V, W or Y, preferably to A, C, F, R or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position E791 to A, C, D, F, G, H, I, K, L, M, N, P, Q, R, S, T, V, W or Y, preferably to A, D, F, M, S, T, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V792 to A, C, D, E, F, G, H, I, K, L, M, N, P, Q, R, S, T, W or Y, preferably to C, G, L, S or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position D793 to A, C, E, F, G, H, I, K, L, M, N, P, Q, R, S, T, V, W or Y, preferably to C, F, H, K, N or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V794 to A, C, D, E, F, G, H, I, K, L, M, N, P, Q, R, S, T, W or Y, preferably to C, D, L, Q, T or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T795 to A, C, D, E, F, G, H, I, K, L, M, N, P, Q, R, S, V, W or Y, preferably to P or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position D796 to A, C, E, F, G, H, I, K, L, M, N, P, Q, R, S, T, V, W or Y, preferably to M, Q, S or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A797 to C, D, E, F, G, H, I, K, L, M, N, P, Q, R, S, T, V, W or Y, preferably to H or K, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position N798 to A, C, D, E, F, G, H, I, K, L, M, P, Q, R, S, T, V, W or Y, preferably to G, I, P or Q, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G799 to A, C, D, E, F, H, I, K, L, M, N, P, Q, R, S, T, V, W or Y, preferably to D, K, L, M, Q or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position H800 to A, C, D, E, F, G, I, K, L, M, N, P, Q, R, S, T, V, W or Y, preferably to A, F, G, L, S or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position 1801 to A, C, D, E, F, G, H, K, L, M, N, P, Q, R, S, T, V, W or Y, preferably to C, E or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V802 to A, C, D, E, F, G, H, I, K, L, M, N, P, Q, R, S, T, W or Y, preferably to E, I, P, S or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position P803 to A, C, D, E, F, G, H, I, K, L, M, N, Q, R, S, T, V, W or Y, preferably to A, F, G, K, S or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position D804 to A, C, E, F, G, H, I, K, L, M, N, P, Q, R, S, T, V, W or Y, preferably to E, G, K, N or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A805 to C, D, E, F, G, H, I, K, L, M, N, P, Q, R, S, T, V, W or Y, preferably to C, F, G, I, N or P, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A806 to C, D, E, F, G, H, I, K, L, M, N, P, Q, R, S, T, V, W or Y, preferably to F, I or Q, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position N807 to A, C, D, E, F, G, H, I, K, L, M, P, Q, R, S, T, V, W or Y, preferably to F, Q, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position R808 to A, C, D, E, F, G, H, I, K, L, M, N, P, Q, S, T, V, W or Y, preferably to C, F, G, I, N, P or Q, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V809 to A, C, D, E, F, G, H, I, K, L, M, N, P, Q, R, S, T, W or Y, preferably to A, C, L, M or P, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T810 to A, C, D, E, F, G, H, I, K, L, M, N, P, Q, R, S, V, W or Y, preferably to L, P, Q, R or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position F811 to A, C, D, E, G, H, I, K, L, M, N, P, Q, R, S, T, V, W or Y, preferably to L or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position D812 to A, C, E, F, G, H, I, K, L, M, N, P, Q, R, S, T, V, W or Y, preferably to E, F, I or Q, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V813 to A, C, D, E, F, G, H, I, K, L, M, N, P, Q, R, S, T, W or Y, preferably to F, H, T, W or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position K814 to A, C, D, E, F, G, H, I, L, M, N, P, Q, R, S, T, V, W or Y, preferably to G, H, I, L or P, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G815 to A, C, D, E, F, H, I, K, L, M, N, P, Q, R, S, T, V, W or Y, preferably to A, F, M, P or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A816 to C, D, E, F, G, H, I, K, L, M, N, P, Q, R, S, T, V, W or Y, preferably to C, D, F, I, N, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G817 to A, C, D, E, F, H, I, K, L, M, N, P, Q, R, S, T, V, W or Y, preferably to C, H, I, N or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position K818 to A, C, D, E, F, G, H, I, L, M, N, P, Q, R, S, T, V, W or Y, preferably to D, F, L, Q, R, S, V, W or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position L819 to A, C, D, E, F, G, H, I, K, M, N, P, Q, R, S, T, V, W or Y, preferably to F or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V820 to A, C, D, E, F, G, H, I, K, L, M, N, P, Q, R, S, T, W or Y, preferably to C, F, I, K, R or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G821 to A, C, D, E, F, H, I, K, L, M, N, P, Q, R, S, T, V, W or Y, preferably to A, C, E, F, I, K, M, N, V or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V822 to A, C, D, E, F, G, H, I, K, L, M, N, P, Q, R, S, T, W or Y, preferably to A, D, E or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position D823 to A, C, E, F, G, H, I, K, L, M, N, P, Q, R, S, T, V, W or Y, preferably to E, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position N824 to A, C, D, E, F, G, H, I, K, L, M, P, Q, R, S, T, V, W or Y, preferably to A, C, G or Q, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G825 to A, C, D, E, F, H, I, K, L, M, N, P, Q, R, S, T, V, W or Y, preferably to A, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position S826 to A, C, D, E, F, G, H, I, K, L, M, N, P, Q, R, T, V, W or Y, preferably to A, F, G, I, L, R or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position S827 to A, C, D, E, F, G, H, I, K, L, M, N, P, Q, R, T, V, W or Y, preferably to C or Q, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position P828 to A, C, D, E, F, G, H, I, K, L, M, N, Q, R, S, T, V, W or Y, preferably to C, G, I, L or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position D829 to A, C, E, F, G, H, I, K, L, M, N, P, Q, R, S, T, V, W or Y, preferably to C, I, S, T or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position H830 to A, C, D, E, F, G, I, K, L, M, N, P, Q, R, S, T, V, W or Y, preferably to E, G, M, P, Q, R or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position D831 to A, C, E, F, G, H, I, K, L, M, N, P, Q, R, S, T, V, W or Y, preferably to A, F, G, I, M, P, R or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position S832 to A, C, D, E, F, G, H, I, K, L, M, N, P, Q, R, T, V, W or Y, preferably to E, F, G, L, M, P, R, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position Y833 to A, C, D, E, F, G, H, I, K, L, M, N, P, Q, R, S, T, V or W, preferably to C, D, E, I, K, N, P or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position Q834 to A, C, D, E, F, G, H, I, K, L, M, N, P, R, S, T, V, W or Y, preferably to F, G or M, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A835 to C, D, E, F, G, H, I, K, L, M, N, P, Q, R, S, T, V, W or Y, preferably to D, E, F, H, K or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position D836 to A, C, E, F, G, H, I, K, L, M, N, P, Q, R, S, T, V, W or Y, preferably to C, E, H, Q, R, S, T, V, W or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position N837 to A, C, D, E, F, G, H, I, K, L, M, P, Q, R, S, T, V, W or Y, preferably to D, F, G, H, L, P or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position R838 to A, C, D, E, F, G, H, I, K, L, M, N, P, Q, S, T, V, W or Y, preferably to D, F, G, K, M, N, S or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position K839 to A, C, D, E, F, G, H, I, L, M, N, P, Q, R, S, T, V, W or Y, preferably to A, C, D, E, G, L, N, P or R, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A840 to C, D, E, F, G, H, I, K, L, M, N, P, Q, R, S, T, V, W or Y, preferably to G, I, P or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position F841 to A, C, D, E, G, H, I, K, L, M, N, P, Q, R, S, T, V, W or Y, preferably to C, D, I, K or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position S842 to A, C, D, E, F, G, H, I, K, L, M, N, P, Q, R, T, V, W or Y, preferably to A, L or M, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G843 to A, C, D, E, F, H, I, K, L, M, N, P, Q, R, S, T, V, W or Y, preferably to A or C, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position K844 to A, C, D, E, F, G, H, I, L, M, N, P, Q, R, S, T, V, W or Y, preferably to A, G, L, W or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V845 to A, C, D, E, F, G, H, I, K, L, M, N, P, Q, R, S, T, W or Y, preferably to N or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position L846 to A, C, D, E, F, G, H, I, K, M, N, P, Q, R, S, T, V, W or Y, preferably to G, I, M or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A847 to C, D, E, F, G, H, I, K, L, M, N, P, Q, R, S, T, V, W or Y, preferably to L or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position 1848 to A, C, D, E, F, G, H, K, L, M, N, P, Q, R, S, T, V, W or Y, preferably to M, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V849 to A, C, D, E, F, G, H, I, K, L, M, N, P, Q, R, S, T, W or Y, preferably to A, L, S or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position Q850 to A, C, D, E, F, G, H, I, K, L, M, N, P, R, S, T, V, W or Y, preferably to C, G, I, L, T, V or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position S851 to A, C, D, E, F, G, H, I, K, L, M, N, P, Q, R, T, V, W or Y, preferably to C, D, E, L or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T852 to A, C, D, E, F, G, H, I, K, L, M, N, P, Q, R, S, V, W or Y, preferably to D, G or L, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position K853 to A, C, D, E, F, G, H, I, L, M, N, P, Q, R, S, T, V, W or Y, preferably to N, P, Q or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position E854 to A, C, D, F, G, H, I, K, L, M, N, P, Q, R, S, T, V, W or Y, preferably to C, I, M or R, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A855 to C, D, E, F, G, H, I, K, L, M, N, P, Q, R, S, T, V, W or Y, preferably to K, V or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position E857 to A, C, D, F, G, H, I, K, L, M, N, P, Q, R, S, T, V, W or Y, preferably to P or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position 1858 to A, C, D, E, F, G, H, K, L, M, N, P, Q, R, S, T, V, W or Y, preferably to D, E, F, G, K, M, P, Q or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T859 to A, C, D, E, F, G, H, I, K, L, M, N, P, Q, R, S, V, W or Y, preferably to V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V860 to A, C, D, E, F, G, H, I, K, L, M, N, P, Q, R, S, T, W or Y, preferably to T or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T861 to A, C, D, E, F, G, H, I, K, L, M, N, P, Q, R, S, V, W or Y, preferably to F, I, Q, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A862 to C, D, E, F, G, H, I, K, L, M, N, P, Q, R, S, T, V, W or Y, preferably to C, P or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position K863 to A, C, D, E, F, G, H, I, L, M, N, P, Q, R, S, T, V, W or Y, preferably to F, I, L, N or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A864 to C, D, E, F, G, H, I, K, L, M, N, P, Q, R, S, T, V, W or Y, preferably to E, H, K or L, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position D865 to A, C, E, F, G, H, I, K, L, M, N, P, Q, R, S, T, V, W or Y, preferably to A or G, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G866 to A, C, D, E, F, H, I, K, L, M, N, P, Q, R, S, T, V, W or Y, preferably to H or R, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position L867 to A, C, D, E, F, G, H, I, K, M, N, P, Q, R, S, T, V, W or Y, preferably to W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position S870 to A, C, D, E, F, G, H, I, K, L, M, N, P, Q, R, T, V, W or Y, preferably to E, M, R or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T871 to A, C, D, E, F, G, H, I, K, L, M, N, P, Q, R, S, V, W or Y, preferably to P, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V872 to A, C, D, E, F, G, H, I, K, L, M, N, P, Q, R, S, T, W or Y, preferably to C or G, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position K873 to A, C, D, E, F, G, H, I, L, M, N, P, Q, R, S, T, V, W or Y, preferably to G or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position 1874 to A, C, D, E, F, G, H, K, L, M, N, P, Q, R, S, T, V, W or Y, preferably to G, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A875 to C, D, E, F, G, H, I, K, L, M, N, P, Q, R, S, T, V, W or Y, preferably to R, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T877 to A, C, D, E, F, G, H, I, K, L, M, N, P, Q, R, S, V, W or Y, preferably to A, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V879 to A, C, D, E, F, G, H, I, K, L, M, N, P, Q, R, S, T, W or Y, preferably to P or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position P880 to A, C, D, E, F, G, H, I, K, L, M, N, Q, R, S, T, V, W or Y, preferably to S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G881 to A, C, D, E, F, H, I, K, L, M, N, P, Q, R, S, T, V, W or Y, preferably to W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T882 to A, C, D, E, F, G, H, I, K, L, M, N, P, Q, R, S, V, W or Y, preferably to A, M or R, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position S883 to A, C, D, E, F, G, H, I, K, L, M, N, P, Q, R, T, V, W or Y, preferably to L, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T884 to A, C, D, E, F, G, H, I, K, L, M, N, P, Q, R, S, V, W or Y, preferably to A, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position E885 to A, C, D, F, G, H, I, K, L, M, N, P, Q, R, S, T, V, W or Y, preferably to V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position K886 to A, C, D, E, F, G, H, I, L, M, N, P, Q, R, S, T, V, W or Y, preferably to E, L, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T887 to A, C, D, E, F, G, H, I, K, L, M, N, P, Q, R, S, V, W or Y, preferably to A, D, F, G, N, R or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V888 to A, C, D, E, F, G, H, I, K, L, M, N, P, Q, R, S, T, W or Y, preferably to A, D or G, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position R889 to A, C, D, E, F, G, H, I, K, L, M, N, P, Q, S, T, V, W or Y, preferably to G, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position Y892 to A, C, D, E, F, G, H, I, K, L, M, N, P, Q, R, S, T, V or W, preferably to D, P or R, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position Y893 to A, C, D, E, F, G, H, I, K, L, M, N, P, Q, R, S, T, V or W, preferably to E or G, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position S894 to A, C, D, E, F, G, H, I, K, L, M, N, P, Q, R, T, V, W or Y, preferably to D or G, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position R895 to A, C, D, E, F, G, H, I, K, L, M, N, P, Q, S, T, V, W or Y, preferably to M, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position N896 to A, C, D, E, F, G, H, I, K, L, M, P, Q, R, S, T, V, W or Y, preferably to M, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position Y897 to A, C, D, E, F, G, H, I, K, L, M, N, P, Q, R, S, T, V or W, preferably to V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position Y898 to A, C, D, E, F, G, H, I, K, L, M, N, P, Q, R, S, T, V or W, preferably to T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V899 to A, C, D, E, F, G, H, I, K, L, M, N, P, Q, R, S, T, W or Y, preferably to G, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position K900 to A, C, D, E, F, G, H, I, L, M, N, P, Q, R, S, T, V, W or Y, preferably to E or G, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T901 to A, C, D, E, F, G, H, I, K, L, M, N, P, Q, R, S, V, W or Y, preferably to G, Q, R, V or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G902 to A, C, D, E, F, H, I, K, L, M, N, P, Q, R, S, T, V, W or Y, preferably to A, D, F, L, P, Q, R, S or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position N903 to A, C, D, E, F, G, H, I, K, L, M, P, Q, R, S, T, V, W or Y, preferably to D or I, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position K904 to A, C, D, E, F, G, H, I, L, M, N, P, Q, R, S, T, V, W or Y, preferably to D, E, M, N, R, S, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position P905 to A, C, D, E, F, G, H, I, K, L, M, N, Q, R, S, T, V, W or Y, preferably to A, C, R, V, W or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position 1906 to A, C, D, E, F, G, H, K, L, M, N, P, Q, R, S, T, V, W or Y, preferably to A, D, N, S, T, W or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position L907 to A, C, D, E, F, G, H, I, K, M, N, P, Q, R, S, T, V, W or Y, preferably to F, S or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position P908 to A, C, D, E, F, G, H, I, K, L, M, N, Q, R, S, T, V, W or Y, preferably to C, D, G, I, L, M or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position S909 to A, C, D, E, F, G, H, I, K, L, M, N, P, Q, R, T, V, W or Y, preferably to E, F, G, W or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position D910 to A, C, E, F, G, H, I, K, L, M, N, P, Q, R, S, T, V, W or Y, preferably to C, I, S or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V911 to A, C, D, E, F, G, H, I, K, L, M, N, P, Q, R, S, T, W or Y, preferably to A or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position E912 to A, C, D, F, G, H, I, K, L, M, N, P, Q, R, S, T, V, W or Y, preferably to A, K, L, T or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V913 to A, C, D, E, F, G, H, I, K, L, M, N, P, Q, R, S, T, W or Y, preferably to G, Q, R or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position R914 to A, C, D, E, F, G, H, I, K, L, M, N, P, Q, S, T, V, W or Y, preferably to A, E, F, I, K, V, W or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position Y915 to A, C, D, E, F, G, H, I, K, L, M, N, P, Q, R, S, T, V or W, preferably to A, C, G, I, M, Q or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position S916 to A, C, D, E, F, G, H, I, K, L, M, N, P, Q, R, T, V, W or Y, preferably to G, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position D917 to A, C, E, F, G, H, I, K, L, M, N, P, Q, R, S, T, V, W or Y, preferably to C, E, F, L, M, Q or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G918 to A, C, D, E, F, H, I, K, L, M, N, P, Q, R, S, T, V, W or Y, preferably to E, H, T, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T919 to A, C, D, E, F, G, H, I, K, L, M, N, P, Q, R, S, V, W or Y, preferably to D, K, Q, W or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position S920 to A, C, D, E, F, G, H, I, K, L, M, N, P, Q, R, T, V, W or Y, preferably to C, E, M, P, R, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position D921 to A, C, E, F, G, H, I, K, L, M, N, P, Q, R, S, T, V, W or Y, preferably to C, P, Q or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position R922 to A, C, D, E, F, G, H, I, K, L, M, N, P, Q, S, T, V, W or Y, preferably to A, G, M, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position Q923 to A, C, D, E, F, G, H, I, K, L, M, N, P, R, S, T, V, W or Y, preferably to A, C, E, L, M, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position N924 to A, C, D, E, F, G, H, I, K, L, M, P, Q, R, S, T, V, W or Y, preferably to A, L, P, Q, S or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V925 to A, C, D, E, F, G, H, I, K, L, M, N, P, Q, R, S, T, W or Y, preferably to A, C, E, G, K, N, S or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T926 to A, C, D, E, F, G, H, I, K, L, M, N, P, Q, R, S, V, W or Y, preferably to G, P, R, S, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position W927 to A, C, D, E, F, G, H, I, K, L, M, N, P, Q, R, S, T, V or Y, preferably to C, G or P, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position D928 to A, C, E, F, G, H, I, K, L, M, N, P, Q, R, S, T, V, W or Y, preferably to A, E, H, L or Q, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A929 to C, D, E, F, G, H, I, K, L, M, N, P, Q, R, S, T, V, W or Y, preferably to C, P or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V930 to A, C, D, E, F, G, H, I, K, L, M, N, P, Q, R, S, T, W or Y, preferably to A, E, I, K, M or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position S931 to A, C, D, E, F, G, H, I, K, L, M, N, P, Q, R, T, V, W or Y, preferably to G, P or R, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position D932 to A, C, E, F, G, H, I, K, L, M, N, P, Q, R, S, T, V, W or Y, preferably to F, R, S, T or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position D933 to A, C, E, F, G, H, I, K, L, M, N, P, Q, R, S, T, V, W or Y, preferably to I, R or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position Q934 to A, C, D, E, F, G, H, I, K, L, M, N, P, R, S, T, V, W or Y, preferably to S or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position 1935 to A, C, D, E, F, G, H, K, L, M, N, P, Q, R, S, T, V, W or Y, preferably to A, C, D, E, L, P, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A936 to C, D, E, F, G, H, I, K, L, M, N, P, Q, R, S, T, V, W or Y, preferably to I, L, Q, R or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position K937 to A, C, D, E, F, G, H, I, L, M, N, P, Q, R, S, T, V, W or Y, preferably to G, I, M, P, Q, R or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A938 to C, D, E, F, G, H, I, K, L, M, N, P, Q, R, S, T, V, W or Y, preferably to C, H, N, T, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G939 to A, C, D, E, F, H, I, K, L, M, N, P, Q, R, S, T, V, W or Y, preferably to D or K, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position S940 to A, C, D, E, F, G, H, I, K, L, M, N, P, Q, R, T, V, W or Y, preferably to C, E, M, R, T, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position F941 to A, C, D, E, G, H, I, K, L, M, N, P, Q, R, S, T, V, W or Y, preferably to C, M or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position S942 to A, C, D, E, F, G, H, I, K, L, M, N, P, Q, R, T, V, W or Y, preferably to A, E, K, L, P, T or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V943 to A, C, D, E, F, G, H, I, K, L, M, N, P, Q, R, S, T, W or Y, preferably to A, G, H, Q or R, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A944 to C, D, E, F, G, H, I, K, L, M, N, P, Q, R, S, T, V, W or Y, preferably to D, G, H, P, R or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G945 to A, C, D, E, F, H, I, K, L, M, N, P, Q, R, S, T, V, W or Y, preferably to E, P or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T946 to A, C, D, E, F, G, H, I, K, L, M, N, P, Q, R, S, V, W or Y, preferably to A, E, G, L, P, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V947 to A, C, D, E, F, G, H, I, K, L, M, N, P, Q, R, S, T, W or Y, preferably to G, H, L, M, P, R or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A948 to C, D, E, F, G, H, I, K, L, M, N, P, Q, R, S, T, V, W or Y, preferably to C, I, R or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G949 to A, C, D, E, F, H, I, K, L, M, N, P, Q, R, S, T, V, W or Y, preferably to A, F or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position Q950 to A, C, D, E, F, G, H, I, K, L, M, N, P, R, S, T, V, W or Y, preferably to D, G, K, M or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position K951 to A, C, D, E, F, G, H, I, L, M, N, P, Q, R, S, T, V, W or Y, preferably to D, G, P, Q, S, W or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position 1952 to A, C, D, E, F, G, H, K, L, M, N, P, Q, R, S, T, V, W or Y, preferably to H or Q, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position S953 to A, C, D, E, F, G, H, I, K, L, M, N, P, Q, R, T, V, W or Y, preferably to F, M, N, R or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V954 to A, C, D, E, F, G, H, I, K, L, M, N, P, Q, R, S, T, W or Y, preferably to D, L, Q, S or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position R955 to A, C, D, E, F, G, H, I, K, L, M, N, P, Q, S, T, V, W or Y, preferably to A, C, E, K, Q or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V956 to A, C, D, E, F, G, H, I, K, L, M, N, P, Q, R, S, T, W or Y, preferably to A, D, G, H, I, M, Q or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T957 to A, C, D, E, F, G, H, I, K, L, M, N, P, Q, R, S, V, W or Y, preferably to D, S or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position M958 to A, C, D, E, F, G, H, I, K, L, N, P, Q, R, S, T, V, W or Y, preferably to D, I or K, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position 1959 to A, C, D, E, F, G, H, K, L, M, N, P, Q, R, S, T, V, W or Y, preferably to A, L, S, V or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position D960 to A, C, E, F, G, H, I, K, L, M, N, P, Q, R, S, T, V, W or Y, preferably to G, H, L, P, S or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position E961 to A, C, D, F, G, H, I, K, L, M, N, P, Q, R, S, T, V, W or Y, preferably to D, F, K, P, S or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position 1962 to A, C, D, E, F, G, H, K, L, M, N, P, Q, R, S, T, V, W or Y, preferably to A, C, D, G, K, N, Q or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G963 to A, C, D, E, F, H, I, K, L, M, N, P, Q, R, S, T, V, W or Y, preferably to C, E, L or P, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A964 to C, D, E, F, G, H, I, K, L, M, N, P, Q, R, S, T, V, W or Y, preferably to C, E or H, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position L965 to A, C, D, E, F, G, H, I, K, M, N, P, Q, R, S, T, V, W or Y, preferably to C, E, G, K, M, P, Q, S, V or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position L966 to A, C, D, E, F, G, H, I, K, M, N, P, Q, R, S, T, V, W or Y, preferably to A, G, H, K, N, P, Q, S, T or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position N967 to A, C, D, E, F, G, H, I, K, L, M, P, Q, R, S, T, V, W or Y, preferably to C, D, I, L, M, P, S, T or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position Y968 to A, C, D, E, F, G, H, I, K, L, M, N, P, Q, R, S, T, V or W, preferably to G, L, Q or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position S969 to A, C, D, E, F, G, H, I, K, L, M, N, P, Q, R, T, V, W or Y, preferably to C, D, G, H, I, L, M, P, Q or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A970 to C, D, E, F, G, H, I, K, L, M, N, P, Q, R, S, T, V, W or Y, preferably to I or L, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position S971 to A, C, D, E, F, G, H, I, K, L, M, N, P, Q, R, T, V, W or Y, preferably to E, F, G, H, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position P973 to A, C, D, E, F, G, H, I, K, L, M, N, Q, R, S, T, V, W or Y, preferably to C, D, K, N, Q, R, V, W or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V974 to A, C, D, E, F, G, H, I, K, L, M, N, P, Q, R, S, T, W or Y, preferably to C, E, G, N, T or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G975 to A, C, D, E, F, H, I, K, L, M, N, P, Q, R, S, T, V, W or Y, preferably to D, F, K, L, Q or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T976 to A, C, D, E, F, G, H, I, K, L, M, N, P, Q, R, S, V, W or Y, preferably to D, F, G, K, L, P, S or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position P977 to A, C, D, E, F, G, H, I, K, L, M, N, Q, R, S, T, V, W or Y, preferably to A, C, K, R, T or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A978 to C, D, E, F, G, H, I, K, L, M, N, P, Q, R, S, T, V, W or Y, preferably to F, G, M, N, P, R, S or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V979 to A, C, D, E, F, G, H, I, K, L, M, N, P, Q, R, S, T, W or Y, preferably to G, N, R or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position L980 to A, C, D, E, F, G, H, I, K, M, N, P, Q, R, S, T, V, W or Y, preferably to A, F, H, I, K, N, Q, T or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position P981 to A, C, D, E, F, G, H, I, K, L, M, N, Q, R, S, T, V, W or Y, preferably to L or M, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G982 to A, C, D, E, F, H, I, K, L, M, N, P, Q, R, S, T, V, W or Y, preferably to A, H, M, P, Q or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position R984 to A, C, D, E, F, G, H, I, K, L, M, N, P, Q, S, T, V, W or Y, preferably to P or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position P985 to A, C, D, E, F, G, H, I, K, L, M, N, Q, R, S, T, V, W or Y, preferably to E, F, H, K, L or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A986 to C, D, E, F, G, H, I, K, L, M, N, P, Q, R, S, T, V, W or Y, preferably to C, E, F, I, K, L, M, N, S or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V987 to A, C, D, E, F, G, H, I, K, L, M, N, P, Q, R, S, T, W or Y, preferably to A, C, F, I, K, L, Q or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position L988 to A, C, D, E, F, G, H, I, K, M, N, P, Q, R, S, T, V, W or Y, preferably to A, C, E, G, H, M, Q, R, S, V or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position P989 to A, C, D, E, F, G, H, I, K, L, M, N, Q, R, S, T, V, W or Y, preferably to A, C, D, G, H, I, M, N, Q or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position D990 to A, C, E, F, G, H, I, K, L, M, N, P, Q, R, S, T, V, W or Y, preferably to F, P, S or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G991 to A, C, D, E, F, H, I, K, L, M, N, P, Q, R, S, T, V, W or Y, preferably to C, F, H, K, P or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T992 to A, C, D, E, F, G, H, I, K, L, M, N, P, Q, R, S, V, W or Y, preferably to E, H, M, N or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V993 to A, C, D, E, F, G, H, I, K, L, M, N, P, Q, R, S, T, W or Y, preferably to D, G, N or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T994 to A, C, D, E, F, G, H, I, K, L, M, N, P, Q, R, S, V, W or Y, preferably to I, S or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position S995 to A, C, D, E, F, G, H, I, K, L, M, N, P, Q, R, T, V, W or Y, preferably to E, L, R or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A996 to C, D, E, F, G, H, I, K, L, M, N, P, Q, R, S, T, V, W or Y, preferably to Q, R or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position N997 to A, C, D, E, F, G, H, I, K, L, M, P, Q, R, S, T, V, W or Y, preferably to A, C, E, K, L, S, V, W or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position F998 to A, C, D, E, G, H, I, K, L, M, N, P, Q, R, S, T, V, W or Y, preferably to M or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A999 to C, D, E, F, G, H, I, K, L, M, N, P, Q, R, S, T, V, W or Y, preferably to F, G, L, M, R or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V1000 to A, C, D, E, F, G, H, I, K, L, M, N, P, Q, R, S, T, W or Y, preferably to C, L, M, N, P or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position D1001 to A, C, E, F, G, H, I, K, L, M, N, P, Q, R, S, T, V, W or Y, preferably to G, K, L, M, Q, S, T, V or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position W1002 to A, C, D, E, F, G, H, I, K, L, M, N, P, Q, R, S, T, V or Y, preferably to A, D, E, H, N, P, Q or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T1003 to A, C, D, E, F, G, H, I, K, L, M, N, P, Q, R, S, V, W or Y, preferably to F, G, L, N, P, R, S, W or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position K1004 to A, C, D, E, F, G, H, I, L, M, N, P, Q, R, S, T, V, W or Y, preferably to D, E, F, G, H, M, P, R, S or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position P1005 to A, C, D, E, F, G, H, I, K, L, M, N, Q, R, S, T, V, W or Y, preferably to I, N, Q, V or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A1006 to C, D, E, F, G, H, I, K, L, M, N, P, Q, R, S, T, V, W or Y, preferably to C, I, N, P, S, V, W or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position D1007 to A, C, E, F, G, H, I, K, L, M, N, P, Q, R, S, T, V, W or Y, preferably to C, L, P or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T1008 to A, C, D, E, F, G, H, I, K, L, M, N, P, Q, R, S, V, W or Y, preferably to G, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V1009 to A, C, D, E, F, G, H, I, K, L, M, N, P, Q, R, S, T, W or Y, preferably to G or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position Y1010 to A, C, D, E, F, G, H, I, K, L, M, N, P, Q, R, S, T, V or W, preferably to A, P, R or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position N1011 to A, C, D, E, F, G, H, I, K, L, M, P, Q, R, S, T, V, W or Y, preferably to A, S, T or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T1012 to A, C, D, E, F, G, H, I, K, L, M, N, P, Q, R, S, V, W or Y, preferably to E, H, I, Q or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A1013 to C, D, E, F, G, H, I, K, L, M, N, P, Q, R, S, T, V, W or Y, preferably to D, K, Q, T or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G1014 to A, C, D, E, F, H, I, K, L, M, N, P, Q, R, S, T, V, W or Y, preferably to E, I, L, M, V, W or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T1015 to A, C, D, E, F, G, H, I, K, L, M, N, P, Q, R, S, V, W or Y, preferably to A, F, G or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V1016 to A, C, D, E, F, G, H, I, K, L, M, N, P, Q, R, S, T, W or Y, preferably to C, D or P, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position K1017 to A, C, D, E, F, G, H, I, L, M, N, P, Q, R, S, T, V, W or Y, preferably to E or G, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V1018 to A, C, D, E, F, G, H, I, K, L, M, N, P, Q, R, S, T, W or Y, preferably to I, K, L, M, R, S or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T1021 to A, C, D, E, F, G, H, I, K, L, M, N, P, Q, R, S, V, W or Y, preferably to C, E, F, G, K, L, S or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A1022 to C, D, E, F, G, H, I, K, L, M, N, P, Q, R, S, T, V, W or Y, preferably to H, L, S or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T1023 to A, C, D, E, F, G, H, I, K, L, M, N, P, Q, R, S, V, W or Y, preferably to D, M, Q or R, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V1024 to A, C, D, E, F, G, H, I, K, L, M, N, P, Q, R, S, T, W or Y, preferably to E, G, H, K, N, R, S or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G1026 to A, C, D, E, F, H, I, K, L, M, N, P, Q, R, S, T, V, W or Y, preferably to E, H, L, R, S, V or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position K1027 to A, C, D, E, F, G, H, I, L, M, N, P, Q, R, S, T, V, W or Y, preferably to C, N, Q, R or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position E1028 to A, C, D, F, G, H, I, K, L, M, N, P, Q, R, S, T, V, W or Y, preferably to G, S or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position F1029 to A, C, D, E, G, H, I, K, L, M, N, P, Q, R, S, T, V, W or Y, preferably to I, K, L, P, V, W or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position K1030 to A, C, D, E, F, G, H, I, L, M, N, P, Q, R, S, T, V, W or Y, preferably to D, F, H, L, M or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V1031 to A, C, D, E, F, G, H, I, K, L, M, N, P, Q, R, S, T, W or Y, preferably to H, K or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A1033 to C, D, E, F, G, H, I, K, L, M, N, P, Q, R, S, T, V, W or Y, preferably to G, S or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T1034 to A, C, D, E, F, G, H, I, K, L, M, N, P, Q, R, S, V, W or Y, preferably to G, H, N or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position 11035 to A, C, D, E, F, G, H, K, L, M, N, P, Q, R, S, T, V, W or Y, preferably to D, G or Q, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position R1036 to A, C, D, E, F, G, H, I, K, L, M, N, P, Q, S, T, V, W or Y, preferably to G, L, T or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V1037 to A, C, D, E, F, G, H, I, K, L, M, N, P, Q, R, S, T, W or Y, preferably to C, F, P or Q, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position Q1038 to A, C, D, E, F, G, H, I, K, L, M, N, P, R, S, T, V, W or Y, preferably to A, D or K, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position R1039 to A, C, D, E, F, G, H, I, K, L, M, N, P, Q, S, T, V, W or Y, preferably to S or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position S1040 to A, C, D, E, F, G, H, I, K, L, M, N, P, Q, R, T, V, W or Y, preferably to A, M, N, R or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position Q1041 to A, C, D, E, F, G, H, I, K, L, M, N, P, R, S, T, V, W or Y, preferably to P, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V1042 to A, C, D, E, F, G, H, I, K, L, M, N, P, Q, R, S, T, W or Y, preferably to N, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T1043 to A, C, D, E, F, G, H, I, K, L, M, N, P, Q, R, S, V, W or Y, preferably to F, G, N or R, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position 11044 to A, C, D, E, F, G, H, K, L, M, N, P, Q, R, S, T, V, W or Y, preferably to A or L, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G1045 to A, C, D, E, F, H, I, K, L, M, N, P, Q, R, S, T, V, W or Y, preferably to S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position S1046 to A, C, D, E, F, G, H, I, K, L, M, N, P, Q, R, T, V, W or Y, preferably to I or M, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position S1047 to A, C, D, E, F, G, H, I, K, L, M, N, P, Q, R, T, V, W or Y, preferably to D, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V1048 to A, C, D, E, F, G, H, I, K, L, M, N, P, Q, R, S, T, W or Y, preferably to C, F, G, I, M or Q, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G1050 to A, C, D, E, F, H, I, K, L, M, N, P, Q, R, S, T, V, W or Y, preferably to L, S or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position N1051 to A, C, D, E, F, G, H, I, K, L, M, P, Q, R, S, T, V, W or Y, preferably to A, E or K, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A1052 to C, D, E, F, G, H, I, K, L, M, N, P, Q, R, S, T, V, W or Y, preferably to C, K, M, P or R, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position L1053 to A, C, D, E, F, G, H, I, K, M, N, P, Q, R, S, T, V, W or Y, preferably to A or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position R1054 to A, C, D, E, F, G, H, I, K, L, M, N, P, Q, S, T, V, W or Y, preferably to C, L or N, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position L1055 to A, C, D, E, F, G, H, I, K, M, N, P, Q, R, S, T, V, W or Y, preferably to R or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position Q1057 to A, C, D, E, F, G, H, I, K, L, M, N, P, R, S, T, V, W or Y, preferably to A, E, P or R, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position N1058 to A, C, D, E, F, G, H, I, K, L, M, P, Q, R, S, T, V, W or Y, preferably to R, S, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position 11059 to A, C, D, E, F, G, H, K, L, M, N, P, Q, R, S, T, V, W or Y, preferably to W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position P1060 to A, C, D, E, F, G, H, I, K, L, M, N, Q, R, S, T, V, W or Y, preferably to G, N, Q, S or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A1061 to C, D, E, F, G, H, I, K, L, M, N, P, Q, R, S, T, V, W or Y, preferably to E, G, K or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position D1062 to A, C, E, F, G, H, I, K, L, M, N, P, Q, R, S, T, V, W or Y, preferably to A, F, G, I, L, M, P or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position K1063 to A, C, D, E, F, G, H, I, L, M, N, P, Q, R, S, T, V, W or Y, preferably to D or M, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position Q1064 to A, C, D, E, F, G, H, I, K, L, M, N, P, R, S, T, V, W or Y, preferably to C, M, R, T or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position S1065 to A, C, D, E, F, G, H, I, K, L, M, N, P, Q, R, T, V, W or Y, preferably to A, C, E, G, T or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position D1066 to A, C, E, F, G, H, I, K, L, M, N, P, Q, R, S, T, V, W or Y, preferably to A, G, M, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T1067 to A, C, D, E, F, G, H, I, K, L, M, N, P, Q, R, S, V, W or Y, preferably to G or M, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position L1068 to A, C, D, E, F, G, H, I, K, M, N, P, Q, R, S, T, V, W or Y, preferably to C, E, P, Q or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position D1069 to A, C, E, F, G, H, I, K, L, M, N, P, Q, R, S, T, V, W or Y, preferably to G, K, R or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A1070 to C, D, E, F, G, H, I, K, L, M, N, P, Q, R, S, T, V, W or Y, preferably to P or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position 11071 to A, C, D, E, F, G, H, K, L, M, N, P, Q, R, S, T, V, W or Y, preferably to M, R or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position K1072 to A, C, D, E, F, G, H, I, L, M, N, P, Q, R, S, T, V, W or Y, preferably to E, G, P, Q or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position D1073 to A, C, E, F, G, H, I, K, L, M, N, P, Q, R, S, T, V, W or Y, preferably to F, L, M, P or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G1074 to A, C, D, E, F, H, I, K, L, M, N, P, Q, R, S, T, V, W or Y, preferably to I, L or R, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position S1075 to A, C, D, E, F, G, H, I, K, L, M, N, P, Q, R, T, V, W or Y, preferably to C, G, I, L or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T1076 to A, C, D, E, F, G, H, I, K, L, M, N, P, Q, R, S, V, W or Y, preferably to C, E, H, Q or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T1077 to A, C, D, E, F, G, H, I, K, L, M, N, P, Q, R, S, V, W or Y, preferably to K, L or R, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V1078 to A, C, D, E, F, G, H, I, K, L, M, N, P, Q, R, S, T, W or Y, preferably to D, E, L or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position D1079 to A, C, E, F, G, H, I, K, L, M, N, P, Q, R, S, T, V, W or Y, preferably to G or L, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position N1081 to A, C, D, E, F, G, H, I, K, L, M, P, Q, R, S, T, V, W or Y, preferably to D, E or G, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T1082 to A, C, D, E, F, G, H, I, K, L, M, N, P, Q, R, S, V, W or Y, preferably to A, C, E, F, G, K, N or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G1083 to A, C, D, E, F, H, I, K, L, M, N, P, Q, R, S, T, V, W or Y, preferably to E, F, L, P or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G1084 to A, C, D, E, F, H, I, K, L, M, N, P, Q, R, S, T, V, W or Y, preferably to C, M, V, W or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G1085 to A, C, D, E, F, H, I, K, L, M, N, P, Q, R, S, T, V, W or Y, preferably to A, P, R or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A1086 to C, D, E, F, G, H, I, K, L, M, N, P, Q, R, S, T, V, W or Y, preferably to H, K, Q, R or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position N1087 to A, C, D, E, F, G, H, I, K, L, M, P, Q, R, S, T, V, W or Y, preferably to A, E, I, R, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position P1088 to A, C, D, E, F, G, H, I, K, L, M, N, Q, R, S, T, V, W or Y, preferably to D, E, G, R or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position S1089 to A, C, D, E, F, G, H, I, K, L, M, N, P, Q, R, T, V, W or Y, preferably to C, E, G, K, Q, R or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A1090 to C, D, E, F, G, H, I, K, L, M, N, P, Q, R, S, T, V, W or Y, preferably to F, G, I or K, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position W1091 to A, C, D, E, F, G, H, I, K, L, M, N, P, Q, R, S, T, V or Y, preferably to A, E, G, H, T, V or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T1092 to A, C, D, E, F, G, H, I, K, L, M, N, P, Q, R, S, V, W or Y, preferably to A, E, G, K, Q, S or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position N1093 to A, C, D, E, F, G, H, I, K, L, M, P, Q, R, S, T, V, W or Y, preferably to A, G, L, P, Q, T or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position W1094 to A, C, D, E, F, G, H, I, K, L, M, N, P, Q, R, S, T, V or Y, preferably to D, E, P, R or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A1095 to C, D, E, F, G, H, I, K, L, M, N, P, Q, R, S, T, V, W or Y, preferably to P, R, T or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position Y1096 to A, C, D, E, F, G, H, I, K, L, M, N, P, Q, R, S, T, V or W, preferably to A, D, H, L or R, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position S1097 to A, C, D, E, F, G, H, I, K, L, M, N, P, Q, R, T, V, W or Y, preferably to D, E, K, L, T or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position K1098 to A, C, D, E, F, G, H, I, L, M, N, P, Q, R, S, T, V, W or Y, preferably to D, F, G, Q or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A1099 to C, D, E, F, G, H, I, K, L, M, N, P, Q, R, S, T, V, W or Y, preferably to C, D, F, S, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G1100 to A, C, D, E, F, H, I, K, L, M, N, P, Q, R, S, T, V, W or Y, preferably to D, E, H, M, N or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position H1101 to A, C, D, E, F, G, I, K, L, M, N, P, Q, R, S, T, V, W or Y, preferably to K, L, Q, R or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position N1102 to A, C, D, E, F, G, H, I, K, L, M, P, Q, R, S, T, V, W or Y, preferably to E, F, H, K, L, Q, R or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T1103 to A, C, D, E, F, G, H, I, K, L, M, N, P, Q, R, S, V, W or Y, preferably to A, E, H, S or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A1104 to C, D, E, F, G, H, I, K, L, M, N, P, Q, R, S, T, V, W or Y, preferably to I, K or R, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position E1105 to A, C, D, F, G, H, I, K, L, M, N, P, Q, R, S, T, V, W or Y, preferably to L or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position 11106 to A, C, D, E, F, G, H, K, L, M, N, P, Q, R, S, T, V, W or Y, preferably to T or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T1107 to A, C, D, E, F, G, H, I, K, L, M, N, P, Q, R, S, V, W or Y, preferably to C, M, R or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position F1108 to A, C, D, E, G, H, I, K, L, M, N, P, Q, R, S, T, V, W or Y, preferably to D, K, L, T or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position E1109 to A, C, D, F, G, H, I, K, L, M, N, P, Q, R, S, T, V, W or Y, preferably to A, D, L or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A1111 to C, D, E, F, G, H, I, K, L, M, N, P, Q, R, S, T, V, W or Y, preferably to G or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position E1113 to A, C, D, F, G, H, I, K, L, M, N, P, Q, R, S, T, V, W or Y, preferably to D, G, P or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position Q1114 to A, C, D, E, F, G, H, I, K, L, M, N, P, R, S, T, V, W or Y, preferably to E, I, L, R, S, T or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position Q1115 to A, C, D, E, F, G, H, I, K, L, M, N, P, R, S, T, V, W or Y, preferably to A, K, L, P, T or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position L1116 to A, C, D, E, F, G, H, I, K, M, N, P, Q, R, S, T, V, W or Y, preferably to D, G, H, K, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G1117 to A, C, D, E, F, H, I, K, L, M, N, P, Q, R, S, T, V, W or Y, preferably to E, I, M, R, S, T, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position Q1118 to A, C, D, E, F, G, H, I, K, L, M, N, P, R, S, T, V, W or Y, preferably to A, M, S, T or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position 11119 to A, C, D, E, F, G, H, K, L, M, N, P, Q, R, S, T, V, W or Y, preferably to D, E, G, N or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V1120 to A, C, D, E, F, G, H, I, K, L, M, N, P, Q, R, S, T, W or Y, preferably to N, S or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position M1121 to A, C, D, E, F, G, H, I, K, L, N, P, Q, R, S, T, V, W or Y, preferably to G, K, N, P, S, V or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position Y1122 to A, C, D, E, F, G, H, I, K, L, M, N, P, Q, R, S, T, V or W, preferably to A, C, I, K, R, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position F1123 to A, C, D, E, G, H, I, K, L, M, N, P, Q, R, S, T, V, W or Y, preferably to E, H, R or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position F1124 to A, C, D, E, G, H, I, K, L, M, N, P, Q, R, S, T, V, W or Y, preferably to E, R, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position R1125 to A, C, D, E, F, G, H, I, K, L, M, N, P, Q, S, T, V, W or Y, preferably to D, E, F, K, T, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position D1126 to A, C, E, F, G, H, I, K, L, M, N, P, Q, R, S, T, V, W or Y, preferably to H, K, L or R, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position S1127 to A, C, D, E, F, G, H, I, K, L, M, N, P, Q, R, T, V, W or Y, preferably to F, I, K, M, Q, T or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position N1128 to A, C, D, E, F, G, H, I, K, L, M, P, Q, R, S, T, V, W or Y, preferably to A, C, R, S, T or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A1129 to C, D, E, F, G, H, I, K, L, M, N, P, Q, R, S, T, V, W or Y, preferably to E, L, N, Q, R or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V1130 to A, C, D, E, F, G, H, I, K, L, M, N, P, Q, R, S, T, W or Y, preferably to A, G, P, R or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position R1131 to A, C, D, E, F, G, H, I, K, L, M, N, P, Q, S, T, V, W or Y, preferably to A, N, Q, S or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position F1132 to A, C, D, E, G, H, I, K, L, M, N, P, Q, R, S, T, V, W or Y, preferably to E, K, M, P, Q or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position P1133 to A, C, D, E, F, G, H, I, K, L, M, N, Q, R, S, T, V, W or Y, preferably to D, G, L, Q, R or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position D1134 to A, C, E, F, G, H, I, K, L, M, N, P, Q, R, S, T, V, W or Y, preferably to E, G or L, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A1135 to C, D, E, F, G, H, I, K, L, M, N, P, Q, R, S, T, V, W or Y, preferably to E, K, L, M, S, W or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G1136 to A, C, D, E, F, H, I, K, L, M, N, P, Q, R, S, T, V, W or Y, preferably to A, E, P, Q or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position K1137 to A, C, D, E, F, G, H, I, L, M, N, P, Q, R, S, T, V, W or Y, preferably to A, C, G, L, P, Q, R, S, T or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T1138 to A, C, D, E, F, G, H, I, K, L, M, N, P, Q, R, S, V, W or Y, preferably to R or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position K1139 to A, C, D, E, F, G, H, I, L, M, N, P, Q, R, S, T, V, W or Y, preferably to A, L, R or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position I1140 to A, C, D, E, F, G, H, K, L, M, N, P, Q, R, S, T, V, W or Y, preferably to A, C, G, L, M, P, R or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position Q1141 to A, C, D, E, F, G, H, I, K, L, M, N, P, R, S, T, V, W or Y, preferably to A, C, G, K, N, P, T or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position 11142 to A, C, D, E, F, G, H, K, L, M, N, P, Q, R, S, T, V, W or Y, preferably to E, R, S, W or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position S1143 to A, C, D, E, F, G, H, I, K, L, M, N, P, Q, R, T, V, W or Y, preferably to G, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A1144 to C, D, E, F, G, H, I, K, L, M, N, P, Q, R, S, T, V, W or Y, preferably to C, D, E, N, P, S, T, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position D1145 to A, C, E, F, G, H, I, K, L, M, N, P, Q, R, S, T, V, W or Y, preferably to E, R or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G1146 to A, C, D, E, F, H, I, K, L, M, N, P, Q, R, S, T, V, W or Y, preferably to A, C, D, K, L, R or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position K1147 to A, C, D, E, F, G, H, I, L, M, N, P, Q, R, S, T, V, W or Y, preferably to A, G, T or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position N1148 to A, C, D, E, F, G, H, I, K, L, M, P, Q, R, S, T, V, W or Y, preferably to H, I, K, P, Q, R, S, T or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position W1149 to A, C, D, E, F, G, H, I, K, L, M, N, P, Q, R, S, T, V or Y, preferably to C, G, I, K, N, Q, S, T, V or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T1150 to A, C, D, E, F, G, H, I, K, L, M, N, P, Q, R, S, V, W or Y, preferably to G, K or P, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position D1151 to A, C, E, F, G, H, I, K, L, M, N, P, Q, R, S, T, V, W or Y, preferably to C, G, R, T or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position L1152 to A, C, D, E, F, G, H, I, K, M, N, P, Q, R, S, T, V, W or Y, preferably to A, C, E, Q or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A1153 to C, D, E, F, G, H, I, K, L, M, N, P, Q, R, S, T, V, W or Y, preferably to E, G, K or L, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A1154 to C, D, E, F, G, H, I, K, L, M, N, P, Q, R, S, T, V, W or Y, preferably to C, D, E, G, R or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T1155 to A, C, D, E, F, G, H, I, K, L, M, N, P, Q, R, S, V, W or Y, preferably to E, L, Q or R, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T1157 to A, C, D, E, F, G, H, I, K, L, M, N, P, Q, R, S, V, W or Y, preferably to V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position 11158 to A, C, D, E, F, G, H, K, L, M, N, P, Q, R, S, T, V, W or Y, preferably to R, S or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A1159 to C, D, E, F, G, H, I, K, L, M, N, P, Q, R, S, T, V, W or Y, preferably to C, E, I, P, R or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A1160 to C, D, E, F, G, H, I, K, L, M, N, P, Q, R, S, T, V, W or Y, preferably to K, L, Q or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position Q1161 to A, C, D, E, F, G, H, I, K, L, M, N, P, R, S, T, V, W or Y, preferably to A, P or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position E1162 to A, C, D, F, G, H, I, K, L, M, N, P, Q, R, S, T, V, W or Y, preferably to A, C, D, F, I, N, Q, T, W or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position E1165 to A, C, D, F, G, H, I, K, L, M, N, P, Q, R, S, T, V, W or Y, preferably to D, H, L, M, R, S or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position R1166 to A, C, D, E, F, G, H, I, K, L, M, N, P, Q, S, T, V, W or Y, preferably to D, K or Q, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V1167 to A, C, D, E, F, G, H, I, K, L, M, N, P, Q, R, S, T, W or Y, preferably to A, C, L, P or R, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position K1168 to A, C, D, E, F, G, H, I, L, M, N, P, Q, R, S, T, V, W or Y, preferably to L, Q, R or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position P1169 to A, C, D, E, F, G, H, I, K, L, M, N, Q, R, S, T, V, W or Y, preferably to M, R or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position Y1170 to A, C, D, E, F, G, H, I, K, L, M, N, P, Q, R, S, T, V or W, preferably to E, K, M, Q, R or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T1171 to A, C, D, E, F, G, H, I, K, L, M, N, P, Q, R, S, V, W or Y, preferably to A, G, M, Q, R or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position Y1172 to A, C, D, E, F, G, H, I, K, L, M, N, P, Q, R, S, T, V or W, preferably to D, E, H, I, K, L, S or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position D1173 to A, C, E, F, G, H, I, K, L, M, N, P, Q, R, S, T, V, W or Y, preferably to A, E, F, G, K, L, P, R, T or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position F1174 to A, C, D, E, G, H, I, K, L, M, N, P, Q, R, S, T, V, W or Y, preferably to D, P, Q, R, S, T, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A1175 to C, D, E, F, G, H, I, K, L, M, N, P, Q, R, S, T, V, W or Y, preferably to G, I, N, Q, S, V or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V1177 to A, C, D, E, F, G, H, I, K, L, M, N, P, Q, R, S, T, W or Y, preferably to N, P, S or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G1178 to A, C, D, E, F, H, I, K, L, M, N, P, Q, R, S, T, V, W or Y, preferably to M, Q, S or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A1179 to C, D, E, F, G, H, I, K, L, M, N, P, Q, R, S, T, V, W or Y, preferably to L, P, Q or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T1180 to A, C, D, E, F, G, H, I, K, L, M, N, P, Q, R, S, V, W or Y, preferably to A, G, I, L, M, Q, S or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position F1181 to A, C, D, E, G, H, I, K, L, M, N, P, Q, R, S, T, V, W or Y, preferably to E, L or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V1182 to A, C, D, E, F, G, H, I, K, L, M, N, P, Q, R, S, T, W or Y, preferably to M, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position K1183 to A, C, D, E, F, G, H, I, L, M, N, P, Q, R, S, T, V, W or Y, preferably to A, E, T or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V1184 to A, C, D, E, F, G, H, I, K, L, M, N, P, Q, R, S, T, W or Y, preferably to C, E, K, L, P, Q or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T1185 to A, C, D, E, F, G, H, I, K, L, M, N, P, Q, R, S, V, W or Y, preferably to Q or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V1186 to A, C, D, E, F, G, H, I, K, L, M, N, P, Q, R, S, T, W or Y, preferably to S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position N1188 to A, C, D, E, F, G, H, I, K, L, M, P, Q, R, S, T, V, W or Y, preferably to V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A1189 to C, D, E, F, G, H, I, K, L, M, N, P, Q, R, S, T, V, W or Y, preferably to C, K, T or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position D1190 to A, C, E, F, G, H, I, K, L, M, N, P, Q, R, S, T, V, W or Y, preferably to R, S, T or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T1191 to A, C, D, E, F, G, H, I, K, L, M, N, P, Q, R, S, V, W or Y, preferably to E or L, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T1192 to A, C, D, E, F, G, H, I, K, L, M, N, P, Q, R, S, V, W or Y, preferably to H or P, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T1193 to A, C, D, E, F, G, H, I, K, L, M, N, P, Q, R, S, V, W or Y, preferably to G, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position P1194 to A, C, D, E, F, G, H, I, K, L, M, N, Q, R, S, T, V, W or Y, preferably to A, E, G or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position S1195 to A, C, D, E, F, G, H, I, K, L, M, N, P, Q, R, T, V, W or Y, preferably to G, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V1197 to A, C, D, E, F, G, H, I, K, L, M, N, P, Q, R, S, T, W or Y, preferably to A, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V1198 to A, C, D, E, F, G, H, I, K, L, M, N, P, Q, R, S, T, W or Y, preferably to E, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position C1199 to A, D, E, F, G, H, I, K, L, M, N, P, Q, R, S, T, V, W or Y, preferably to D or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A1200 to C, D, E, F, G, H, I, K, L, M, N, P, Q, R, S, T, V, W or Y, preferably to G, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position L1202 to A, C, D, E, F, G, H, I, K, M, N, P, Q, R, S, T, V, W or Y, preferably to A or C, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T1203 to A, C, D, E, F, G, H, I, K, L, M, N, P, Q, R, S, V, W or Y, preferably to E or K, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position E1204 to A, C, D, F, G, H, I, K, L, M, N, P, Q, R, S, T, V, W or Y, preferably to G or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position 11205 to A, C, D, E, F, G, H, K, L, M, N, P, Q, R, S, T, V, W or Y, preferably to V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position K1208 to A, C, D, E, F, G, H, I, L, M, N, P, Q, R, S, T, V, W or Y, preferably to R, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T1209 to A, C, D, E, F, G, H, I, K, L, M, N, P, Q, R, S, V, W or Y, preferably to A, C, E, K, N, Q, R or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A1210 to C, D, E, F, G, H, I, K, L, M, N, P, Q, R, S, T, V, W or Y, preferably to D, E, G, K, L, Q, R, T or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T1211 to A, C, D, E, F, G, H, I, K, L, M, N, P, Q, R, S, V, W or Y, preferably to C, D, E, G, H, K, P, Q, R, S or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ

In another aspect, the variant comprises a substitution at position K1213 to A, C, D, E, F, G, H, I, L, M, N, P, Q, R, S, T, V, W or Y, preferably to A, D, S, T or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position F1214 to A, C, D, E, G, H, I, K, L, M, N, P, Q, R, S, T, V, W or Y, preferably to A, E, K, L, P, R, S or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V1215 to A, C, D, E, F, G, H, I, K, L, M, N, P, Q, R, S, T, W or Y, preferably to D, E, K, L, Q, S or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T1216 to A, C, D, E, F, G, H, I, K, L, M, N, P, Q, R, S, V, W or Y, preferably to A, L, P, Q or R, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position N1217 to A, C, D, E, F, G, H, I, K, L, M, P, Q, R, S, T, V, W or Y, preferably to A, D, E, F, P, R, S or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T1218 to A, C, D, E, F, G, H, I, K, L, M, N, P, Q, R, S, V, W or Y, preferably to A, C, E, G, Q, S, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position S1219 to A, C, D, E, F, G, H, I, K, L, M, N, P, Q, R, T, V, W or Y, preferably to A, E, F, I, K, R or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A1220 to C, D, E, F, G, H, I, K, L, M, N, P, Q, R, S, T, V, W or Y, preferably to C, G, L, P, R or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A1221 to C, D, E, F, G, H, I, K, L, M, N, P, Q, R, S, T, V, W or Y, preferably to D, G, K, L, R, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position L1222 to A, C, D, E, F, G, H, I, K, M, N, P, Q, R, S, T, V, W or Y, preferably to A, C, E, F, Q, R, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position S1223 to A, C, D, E, F, G, H, I, K, L, M, N, P, Q, R, T, V, W or Y, preferably to C, F, G, K, L or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position S1224 to A, C, D, E, F, G, H, I, K, L, M, N, P, Q, R, T, V, W or Y, preferably to A, D, G, L, M, P, R or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position L1225 to A, C, D, E, F, G, H, I, K, M, N, P, Q, R, S, T, V, W or Y, preferably to C, D, E, F, G, K, P, T, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T1226 to A, C, D, E, F, G, H, I, K, L, M, N, P, Q, R, S, V, W or Y, preferably to A, G, M, P, R, S, V or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V1227 to A, C, D, E, F, G, H, I, K, L, M, N, P, Q, R, S, T, W or Y, preferably to A, C, D, E, G, L, P, Q or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position N1228 to A, C, D, E, F, G, H, I, K, L, M, P, Q, R, S, T, V, W or Y, preferably to A, D, F, K, L or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G1229 to A, C, D, E, F, H, I, K, L, M, N, P, Q, R, S, T, V, W or Y, preferably to A, C, E, Q, S or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T1230 to A, C, D, E, F, G, H, I, K, L, M, N, P, Q, R, S, V, W or Y, preferably to F, H, I, K, L, P, R, S or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position K1231 to A, C, D, E, F, G, H, I, L, M, N, P, Q, R, S, T, V, W or Y, preferably to F, G, L, M, P, S or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V1232 to A, C, D, E, F, G, H, I, K, L, M, N, P, Q, R, S, T, W or Y, preferably to E, K, Q, R, S, T or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position S1233 to A, C, D, E, F, G, H, I, K, L, M, N, P, Q, R, T, V, W or Y, preferably to P or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position D1234 to A, C, E, F, G, H, I, K, L, M, N, P, Q, R, S, T, V, W or Y, preferably to G, K, R or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position S1235 to A, C, D, E, F, G, H, I, K, L, M, N, P, Q, R, T, V, W or Y, preferably to D, E, G, L, P, R, W or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V1236 to A, C, D, E, F, G, H, I, K, L, M, N, P, Q, R, S, T, W or Y, preferably to A, C, G, I, P, Q or R, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position L1237 to A, C, D, E, F, G, H, I, K, M, N, P, Q, R, S, T, V, W or Y, preferably to D, E, R, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A1238 to C, D, E, F, G, H, I, K, L, M, N, P, Q, R, S, T, V, W or Y, preferably to D, E, K, L, N, P, R, S or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A1239 to C, D, E, F, G, H, I, K, L, M, N, P, Q, R, S, T, V, W or Y, preferably to D, P or R, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G1240 to A, C, D, E, F, H, I, K, L, M, N, P, Q, R, S, T, V, W or Y, preferably to D, L, N, Q, S, T or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position S1241 to A, C, D, E, F, G, H, I, K, L, M, N, P, Q, R, T, V, W or Y, preferably to D, G, I, L, M or P, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position Y1242 to A, C, D, E, F, G, H, I, K, L, M, N, P, Q, R, S, T, V or W, preferably to C, E, K, R, S or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position N1243 to A, C, D, E, F, G, H, I, K, L, M, P, Q, R, S, T, V, W or Y, preferably to C, L, M, P, Q, S, T, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T1244 to A, C, D, E, F, G, H, I, K, L, M, N, P, Q, R, S, V, W or Y, preferably to A, D, E, G, L, Q, S, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A1246 to C, D, E, F, G, H, I, K, L, M, N, P, Q, R, S, T, V, W or Y, preferably to F, M, N, P, Q, R, S or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position I1247 to A, C, D, E, F, G, H, K, L, M, N, P, Q, R, S, T, V, W or Y, preferably to A, G, M, Q, R, S, T, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position 11248 to A, C, D, E, F, G, H, K, L, M, N, P, Q, R, S, T, V, W or Y, preferably to A, G, K, L, R, S or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A1249 to C, D, E, F, G, H, I, K, L, M, N, P, Q, R, S, T, V, W or Y, preferably to E, G, H, I, R, T or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position D1250 to A, C, E, F, G, H, I, K, L, M, N, P, Q, R, S, T, V, W or Y, preferably to I, K, S, T, W or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V1251 to A, C, D, E, F, G, H, I, K, L, M, N, P, Q, R, S, T, W or Y, preferably to I, T or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position K1252 to A, C, D, E, F, G, H, I, L, M, N, P, Q, R, S, T, V, W or Y, preferably to D, G, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A1253 to C, D, E, F, G, H, I, K, L, M, N, P, Q, R, S, T, V, W or Y, preferably to P or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position E1254 to A, C, D, F, G, H, I, K, L, M, N, P, Q, R, S, T, V, W or Y, preferably to F, G, H, L, R or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G1255 to A, C, D, E, F, H, I, K, L, M, N, P, Q, R, S, T, V, W or Y, preferably to H, M, S or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position E1256 to A, C, D, F, G, H, I, K, L, M, N, P, Q, R, S, T, V, W or Y, preferably to G, M, N, R, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G1257 to A, C, D, E, F, H, I, K, L, M, N, P, Q, R, S, T, V, W or Y, preferably to F, K, L, Q, R or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position N1258 to A, C, D, E, F, G, H, I, K, L, M, P, Q, R, S, T, V, W or Y, preferably to C, G, H, K or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A1259 to C, D, E, F, G, H, I, K, L, M, N, P, Q, R, S, T, V, W or Y, preferably to K, L or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V1261 to A, C, D, E, F, G, H, I, K, L, M, N, P, Q, R, S, T, W or Y, preferably to I, L, P, Q, R or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T1262 to A, C, D, E, F, G, H, I, K, L, M, N, P, Q, R, S, V, W or Y, preferably to A, F, M, Q or R, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V1263 to A, C, D, E, F, G, H, I, K, L, M, N, P, Q, R, S, T, W or Y, preferably to E, G, Q, R, T or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position L1264 to A, C, D, E, F, G, H, I, K, M, N, P, Q, R, S, T, V, W or Y, preferably to A, E, H, R, S or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position P1265 to A, C, D, E, F, G, H, I, K, L, M, N, Q, R, S, T, V, W or Y, preferably to C, K, L, R, S, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A1266 to C, D, E, F, G, H, I, K, L, M, N, P, Q, R, S, T, V, W or Y, preferably to F, L, P, S or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position H1267 to A, C, D, E, F, G, I, K, L, M, N, P, Q, R, S, T, V, W or Y, preferably to A, E or F, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position N1269 to A, C, D, E, F, G, H, I, K, L, M, P, Q, R, S, T, V, W or Y, preferably to A, E, K, R, S, T or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V1270 to A, C, D, E, F, G, H, I, K, L, M, N, P, Q, R, S, T, W or Y, preferably to D, E, G, I, L, T or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position 11271 to A, C, D, E, F, G, H, K, L, M, N, P, Q, R, S, T, V, W or Y, preferably to A, H or Q, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position R1272 to A, C, D, E, F, G, H, I, K, L, M, N, P, Q, S, T, V, W or Y, preferably to E, F, M, P or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V1273 to A, C, D, E, F, G, H, I, K, L, M, N, P, Q, R, S, T, W or Y, preferably to R, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position 11274 to A, C, D, E, F, G, H, K, L, M, N, P, Q, R, S, T, V, W or Y, preferably to F, M or R, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T1275 to A, C, D, E, F, G, H, I, K, L, M, N, P, Q, R, S, V, W or Y, preferably to A, L or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position E1276 to A, C, D, F, G, H, I, K, L, M, N, P, Q, R, S, T, V, W or Y, preferably to R or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position S1277 to A, C, D, E, F, G, H, I, K, L, M, N, P, Q, R, T, V, W or Y, preferably to L, T or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position E1278 to A, C, D, F, G, H, I, K, L, M, N, P, Q, R, S, T, V, W or Y, preferably to Q or R, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position D1279 to A, C, E, F, G, H, I, K, L, M, N, P, Q, R, S, T, V, W or Y, preferably to G, I, R, T, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position H1280 to A, C, D, E, F, G, I, K, L, M, N, P, Q, R, S, T, V, W or Y, preferably to C, E, G, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position V1281 to A, C, D, E, F, G, H, I, K, L, M, N, P, Q, R, S, T, W or Y, preferably to F, I, S or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T1282 to A, C, D, E, F, G, H, I, K, L, M, N, P, Q, R, S, V, W or Y, preferably to D, L or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position R1283 to A, C, D, E, F, G, H, I, K, L, M, N, P, Q, S, T, V, W or Y, preferably to A, D, E, P or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position K1284 to A, C, D, E, F, G, H, I, L, M, N, P, Q, R, S, T, V, W or Y, preferably to G, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T1285 to A, C, D, E, F, G, H, I, K, L, M, N, P, Q, R, S, V, W or Y, preferably to A, E, F, G, M, R or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position F1286 to A, C, D, E, G, H, I, K, L, M, N, P, Q, R, S, T, V, W or Y, preferably to A, E, P, R, S or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T1287 to A, C, D, E, F, G, H, I, K, L, M, N, P, Q, R, S, V, W or Y, preferably to C, K, L, M, Q, R, S or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position 11288 to A, C, D, E, F, G, H, K, L, M, N, P, Q, R, S, T, V, W or Y, preferably to A, D, F, G or K, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position N1289 to A, C, D, E, F, G, H, I, K, L, M, P, Q, R, S, T, V, W or Y, preferably to A, Q or T, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position L1290 to A, C, D, E, F, G, H, I, K, M, N, P, Q, R, S, T, V, W or Y, preferably to A, G, R, V or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position G1291 to A, C, D, E, F, H, I, K, L, M, N, P, Q, R, S, T, V, W or Y, preferably to K, P, V, W or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position T1292 to A, C, D, E, F, G, H, I, K, L, M, N, P, Q, R, S, V, W or Y, preferably to G, L or Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position E1293 to A, C, D, F, G, H, I, K, L, M, N, P, Q, R, S, T, V, W or Y, preferably to G, K, L, S or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position Q1294 to A, C, D, E, F, G, H, I, K, L, M, N, P, R, S, T, V, W or Y, preferably to E, L, P or W, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position E1295 to A, C, D, F, G, H, I, K, L, M, N, P, Q, R, S, T, V, W or Y, preferably to K, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position F1296 to A, C, D, E, G, H, I, K, L, M, N, P, Q, R, S, T, V, W or Y, preferably to A, G or I, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position P1297 to A, C, D, E, F, G, H, I, K, L, M, N, Q, R, S, T, V, W or Y, preferably to F, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position A1298 to C, D, E, F, G, H, I, K, L, M, N, P, Q, R, S, T, V, W or Y, preferably to Y, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position D1301 to A, C, E, F, G, H, I, K, L, M, N, P, Q, R, S, T, V, W or Y, preferably to I, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position E1302 to A, C, D, F, G, H, I, K, L, M, N, P, Q, R, S, T, V, W or Y, preferably to G, R or S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position R1303 to A, C, D, E, F, G, H, I, K, L, M, N, P, Q, S, T, V, W or Y, preferably to S, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

In another aspect, the variant comprises a substitution at position D1304 to A, C, E, F, G, H, I, K, L, M, N, P, Q, R, S, T, V, W or Y, preferably to A or V, wherein the variant has at least 80%, 85%, 90%, or 95%, preferably at least 96%, 97%, 98% or 99%, more preferably at least 99.5%, 99.7%, 99.8% or 99.9%, but less than 100%, sequence identity to SEQ ID NO: 1.

Preferably, the lactase variants of the present invention have a decreased or an increased galactose inhibition, preferably measured as a galactose inhibition improvement factor (GI-IF) of at least 1.1 or at most 0.9, compared to the lactase of SEQ ID NO: 1.

In a preferred embodiment, the lactase variants of the present invention have a decreased galactose inhibition, preferably measured as a galactose inhibition improvement factor (GI-IF) of at least 1.1, such as at least 1.2, at least 1.3, at least 1.4 or at least 1.5, at least 1.6, at least 1.7, at least 1.8, or at least 1.9.

The present invention also relates to an isolated polynucleotide encoding a variant of the invention. And to a nucleic acid construct or expression vector comprising such polynucleotide. And to a recombinant host cell transformed with such polynucleotide.

The present invention also relates to a method of producing a lactase variant of the invention, comprising:
a. cultivating the recombinant host cell mentioned in the preceding paragraph under conditions suitable for expression of the variant; and
b. recovering the variant.

The invention also relates to a whole broth formulation or cell culture composition comprising a variant of the invention.

### Nucleic Acid Constructs

The present invention also relates to nucleic acid constructs comprising a polynucleotide encoding a variant of the present invention operably linked to one or more control sequences that direct the expression of the coding sequence in a suitable host cell under conditions compatible with the control sequences.

The polynucleotide may be manipulated in a variety of ways to provide for expression of a variant. Manipulation of the polynucleotide prior to its insertion into a vector may be desirable or necessary depending on the expression vector. The techniques for modifying polynucleotides utilizing recombinant DNA methods are well known in the art.

The control sequence may be a promoter, a polynucleotide recognized by a host cell for expression of a polynucleotide encoding a variant of the present invention. The promoter contains transcriptional control sequences that mediate the expression of the variant. The promoter may be any polynucleotide that shows transcriptional activity in the host cell including mutant, truncated, and hybrid promoters, and may be obtained from genes encoding extracellular or intracellular polypeptides either homologous or heterologous to the host cell.

Examples of suitable promoters for directing transcription of the nucleic acid constructs of the present invention in a bacterial host cell are the promoters obtained from the Bacillus amyloliquefaciens alpha-amylase gene (amyQ), Bacillus licheniformis alpha-amylase gene (amyL), Bacillus licheniformis penicillinase gene (penP), Bacillus stearothermophilus maltogenic amylase gene (amyM), Bacillus subtilis levansucrase gene (sacB), Bacillus subtilis xyIA and xyIB genes, Bacillus thuringiensis cryIIIA gene (Agaisse and Lereclus, 1994, Molecular Microbiology 13: 97-107), E. coli lac operon, E. coli trc promoter (Egon et al., 1988, Gene 69: 301-315), Streptomyces coelicolor agarase gene (dagA), and prokaryotic beta-lactamase gene (Villa-Kamaroff et al., 1978, Proc. Natl. Acad. Sci. USA 75: 3727-3731), as well as the tac promoter (DeBoer et al., 1983, Proc. Natl. Acad. Sci. USA 80: 21-25). Further promoters are described in “Useful proteins from recombinant bacteria” in Gilbert et al., 1980, Scientific American 242: 74-94; and in Sambrook et al., 1989, supra. Examples of tandem promoters are disclosed in WO 99/43835.

Examples of suitable promoters for directing transcription of the nucleic acid constructs of the present invention in a filamentous fungal host cell are promoters obtained from the genes for Aspergillus nidulans acetamidase, Aspergillus niger neutral alpha-amylase, Aspergillus niger acid stable alpha-amylase, Aspergillus niger or Aspergillus awamori glucoamylase (glaA), Aspergillus oryzae TAKA amylase, Aspergillus oryzae alkaline protease, Aspergillus oryzae triose phosphate isomerase, Fusarium oxysporum trypsin-like protease (WO 96/00787), Fusarium venenatum amyloglucosidase (WO 00/56900), Fusarium venenatum Daria (WO 00/56900), Fusarium venenatum Quinn (WO 00/56900), Rhizomucor miehei lipase, Rhizomucor miehei aspartic proteinase, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor, as well as the NA2-tpi promoter (a modified promoter from an Aspergillus neutral alpha-amylase gene in which the untranslated leader has been replaced by an untranslated leader from an Aspergillus triose phosphate isomerase gene; non-limiting examples include modified promoters from an Aspergillus niger neutral alpha-amylase gene in which the untranslated leader has been replaced by an untranslated leader from an Aspergillus nidulans or

Aspergillus oryzae triose phosphate isomerase gene); and mutant, truncated, and hybrid promoters thereof. Other promoters are described in U.S. Pat. No. 6,011,147.

In a yeast host, useful promoters are obtained from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH1, ADH2/GAP), Saccharomyces cerevisiae triose phosphate isomerase (TPI), Saccharomyces cerevisiae metallothionein (CUP1), and Saccharomyces cerevisiae 3-phosphoglycerate kinase. Other useful promoters for yeast host cells are described by Romanos et al., 1992, Yeast 8: 423-488.

The control sequence may also be a transcription terminator, which is recognized by a host cell to terminate transcription. The terminator is operably linked to the 3′-terminus of the polynucleotide encoding the variant. Any terminator that is functional in the host cell may be used in the present invention.

Preferred terminators for bacterial host cells are obtained from the genes for Bacillus clausii alkaline protease (aprH), Bacillus licheniformis alpha-amylase (amyL), and Escherichia coli ribosomal RNA (rrnB).

Preferred terminators for filamentous fungal host cells are obtained from the genes for Aspergillus nidulans acetamidase, Aspergillus nidulans anthranilate synthase, Aspergillus niger glucoamylase, Aspergillus niger alpha-glucosidase, Aspergillus oryzae TAKA amylase, Fusarium oxysporum trypsin-like protease, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor.

Preferred terminators for yeast host cells are obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, supra.

The control sequence may also be an mRNA stabilizer region downstream of a promoter and upstream of the coding sequence of a gene which increases expression of the gene.

Examples of suitable mRNA stabilizer regions are obtained from a Bacillus thuringiensis cryIIIA gene (WO 94/25612) and a Bacillus subtilis SP82 gene (Hue et al., 1995, Journal of Bacteriology 177: 3465-3471).

The control sequence may also be a leader, a nontranslated region of an mRNA that is important for translation by the host cell. The leader is operably linked to the 5′-terminus of the polynucleotide encoding the variant. Any leader that is functional in the host cell may be used.

Preferred leaders for filamentous fungal host cells are obtained from the genes for Aspergillus oryzae TAKA amylase and Aspergillus nidulans triose phosphate isomerase.

Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence operably linked to the 3′-terminus of the polynucleotide and, when transcribed, is recognized by the host cell as a signal to add polyadenosine residues to transcribed mRNA. Any polyadenylation sequence that is functional in the host cell may be used.

Preferred polyadenylation sequences for filamentous fungal host cells are obtained from the genes for Aspergillus nidulans anthranilate synthase, Aspergillus niger glucoamylase, Aspergillus niger alpha-glucosidase Aspergillus oryzae TAKA amylase, and Fusarium oxysporum trypsin-like protease.

Useful polyadenylation sequences for yeast host cells are described by Guo and Sherman, 1995, Mol. Cellular Biol. 15: 5983-5990.

The control sequence may also be a signal peptide coding region that encodes a signal peptide linked to the N-terminus of a variant and directs the variant into the cell's secretory pathway. The 5′-end of the coding sequence of the polynucleotide may inherently contain a signal peptide coding sequence naturally linked in translation reading frame with the segment of the coding sequence that encodes the variant. Alternatively, the 5′-end of the coding sequence may contain a signal peptide coding sequence that is foreign to the coding sequence. A foreign signal peptide coding sequence may be required where the coding sequence does not naturally contain a signal peptide coding sequence. Alternatively, a foreign signal peptide coding sequence may simply replace the natural signal peptide coding sequence in order to enhance secretion of the variant. However, any signal peptide coding sequence that directs the expressed variant into the secretory pathway of a host cell may be used.

Effective signal peptide coding sequences for bacterial host cells are the signal peptide coding sequences obtained from the genes for Bacillus NCIB 11837 maltogenic amylase, Bacillus licheniformis subtilisin, Bacillus licheniformis beta-lactamase, Bacillus stearothermophilus alpha-amylase, Bacillus stearothermophilus neutral proteases (nprT, nprS, nprM), and Bacillus subtilis prsA. Further signal peptides are described by Simonen and Palva, 1993, Microbiological Reviews 57: 109-137.

Effective signal peptide coding sequences for filamentous fungal host cells are the signal peptide coding sequences obtained from the genes for Aspergillus niger neutral amylase, Aspergillus niger glucoamylase, Aspergillus oryzae TAKA amylase, Humicola insolens cellulase, Humicola insolens endoglucanase V, Humicola lanuginosa lipase, and Rhizomucor miehei aspartic proteinase.

Useful signal peptides for yeast host cells are obtained from the genes for Saccharomyces cerevisiae alpha-factor and Saccharomyces cerevisiae invertase. Other useful signal peptide coding sequences are described by Romanos et al., 1992, supra.

The control sequence may also be a propeptide coding sequence that encodes a propeptide positioned at the N-terminus of a variant. The resultant polypeptide is known as a proenzyme or propolypeptide (or a zymogen in some cases). A propolypeptide is generally inactive and can be converted to an active variant by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N-terminus of a variant and the signal peptide sequence is positioned next to the N-terminus of the propeptide sequence.

It may also be desirable to add regulatory sequences that regulate expression of the variant relative to the growth of the host cell. Examples of regulatory sequences are those that cause expression of the gene to be turned on or off in response to a chemical or physical stimulus, including the presence of a regulatory compound. Regulatory sequences in prokaryotic systems include the lac, tac, and trp operator systems. In yeast, the ADH2 system or GAL1 system may be used. In filamentous fungi, the Aspergillus niger glucoamylase promoter, Aspergillus oryzae TAKA alpha-amylase promoter, and Aspergillus oryzae glucoamylase promoter, Trichoderma reesei cellobiohydrolase I promoter, and Trichoderma reesei cellobiohydrolase II promoter may be used. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals. In these cases, the polynucleotide encoding the variant would be operably linked to the regulatory sequence.

### Expression Vectors

The present invention also relates to recombinant expression vectors comprising a polynucleotide encoding a variant of the present invention, a promoter, and transcriptional and translational stop signals. The various nucleotide and control sequences may be joined together to produce a recombinant expression vector that may include one or more convenient restriction sites to allow for insertion or substitution of the polynucleotide encoding the variant at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

Examples of bacterial selectable markers are Bacillus licheniformis or Bacillus subtilis dal genes, or markers that confer antibiotic resistance such as ampicillin, chloramphenicol, kanamycin, neomycin, spectinomycin, or tetracycline resistance. Suitable markers for yeast host cells include, but are not limited to, ADE2, HIS3, LEU2, LYS2, MET3, TRP1, and URA3. Selectable markers for use in a filamentous fungal host cell include, but are not limited to, adeA (phosphoribosylaminoimidazole-succinocarboxamide synthase), adeB (phosphoribosyl-aminoimidazole synthase), amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in an Aspergillus cell are Aspergillus nidulans or Aspergillus oryzae amdS and pyrG genes and a Streptomyces hygroscopicus bar gene. Preferred for use in a Trichoderma cell are adeA, adeB, amdS, hph, and pyrG genes.

The selectable marker may be a dual selectable marker system as described in WO 2010/039889. In one aspect, the dual selectable marker is a hph-tk dual selectable marker system.

The vector preferably contains an element(s) that permits integration of the vector into the host cell's genome or autonomous replication of the vector in the cell independent of the genome.

For integration into the host cell genome, the vector may rely on the polynucleotide's sequence encoding the variant or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of bacterial origins of replication are the origins of replication of plasmids pBR322, pUC19, pACYC177, and pACYC184 permitting replication in E. coli, and pUB110, pE194, pTA1060, and pAMβ1 permitting replication in Bacillus.

Examples of origins of replication for use in a yeast host cell are the 2 micron origin of replication, ARS1, ARS4, the combination of ARS1 and CEN3, and the combination of ARS4 and CEN6.

Examples of origins of replication useful in a filamentous fungal cell are AMA1 and ANS1 (Gems et al., 1991, Gene 98: 61-67; Cullen et al., 1987, Nucleic Acids Res. 15: 9163-9175; WO 00/24883). Isolation of the AMA1 gene and construction of plasmids or vectors comprising the gene can be accomplished according to the methods disclosed in WO 00/24883.

More than one copy of a polynucleotide of the present invention may be inserted into a host cell to increase production of a variant. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

### Host Cells

The present invention also relates to recombinant host cells, comprising a polynucleotide encoding a variant of the present invention operably linked to one or more control sequences that direct the production of a variant of the present invention. A construct or vector comprising a polynucleotide is introduced into a host cell so that the construct or vector is maintained as a chromosomal integrant or as a self-replicating extra-chromosomal vector as described earlier.

The term “host cell” encompasses any progeny of a parent cell that is not identical to the parent cell due to mutations that occur during replication. The choice of a host cell will to a large extent depend upon the gene encoding the variant and its source.

The host cell may be any cell useful in the recombinant production of a variant, e.g., a prokaryote or a eukaryote.

The prokaryotic host cell may be any Gram-positive or Gram-negative bacterium. Gram-positive bacteria include, but are not limited to, Bacillus, Clostridium, Enterococcus, Geobacillus, Lactobacillus, Lactococcus, Oceanobacillus, Staphylococcus, Streptococcus, and Streptomyces. Gram-negative bacteria include, but are not limited to, Campylobacter, E. coli, Flavobacterium, Fusobacterium, Helicobacter, Ilyobacter, Neisseria, Pseudomonas, Salmonella, and Ureaplasma.

The bacterial host cell may be any Bacillus cell including, but not limited to, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis cells. The bacterial host cell may also be any Streptococcus cell including, but not limited to, Streptococcus equisimilis, Streptococcus pyogenes, Streptococcus uberis, and Streptococcus equi subsp. Zooepidemicus cells.

The bacterial host cell may also be any Streptomyces cell, including, but not limited to, Streptomyces achromogenes, Streptomyces avermitilis, Streptomyces coelicolor, Streptomyces griseus, and Streptomyces lividans cells.

The introduction of DNA into a Bacillus cell may be effected by protoplast transformation (see, e.g., Chang and Cohen, 1979, Mol. Gen. Genet. 168: 111-115), competent cell transformation (see, e.g., Young and Spizizen, 1961, J. Bacteriol. 81: 823-829, or Dubnau and Davidoff-Abelson, 1971, J. Mol. Biol. 56: 209-221), electroporation (see, e.g., Shigekawa and Dower, 1988, Biotechniques 6: 742-751), or conjugation (see, e.g., Koehler and Thorne, 1987, J. Bacteriol. 169:5271-5278). The introduction of DNA into an E. coli cell may be effected by protoplast transformation (see, e.g., Hanahan, 1983, J. Mol. Biol. 166: 557-580) or electroporation (see, e.g., Dower et al., 1988, Nucleic Acids Res. 16: 6127-6145). The introduction of DNA into a Streptomyces cell may be effected by protoplast transformation, electroporation (see, e.g., Gong et al., 2004, Folia Microbiol. (Praha) 49: 399-405), conjugation (see, e.g., Mazodier et al., 1989, J. Bacteriol. 171: 3583-3585), or transduction (see, e.g., Burke et al., 2001, Proc. Natl. Acad. Sci. USA 98: 6289-6294). The introduction of DNA into a Pseudomonas cell may be effected by electroporation (see, e.g., Choi et al., 2006, J. Microbiol. Methods 64: 391-397), or conjugation (see, e.g., Pinedo and Smets, 2005, Appl. Environ. Microbiol. 71: 51-57). The introduction of DNA into a Streptococcus cell may be effected by natural competence (see, e.g., Perry and Kuramitsu, 1981, Infect. Immun. 32: 1295-1297), protoplast transformation (see, e.g., Catt and Jollick, 1991, Microbios 68: 189-207), electroporation (see, e.g., Buckley et al., 1999, Appl. Environ. Microbiol. 65: 3800-3804), or conjugation (see, e.g., Clewell, 1981, Microbiol. Rev. 45: 409-436). However, any method known in the art for introducing DNA into a host cell can be used.

The host cell may also be a eukaryote, such as a mammalian, insect, plant, or fungal cell.

The host cell may be a fungal cell. “Fungi” as used herein includes the phyla Ascomycota, Basidiomycota, Chytridiomycota, and Zygomycota as well as the Oomycota and all mitosporic fungi (as defined by Hawksworth et al., In, Ainsworth and Bisby's Dictionary of The Fungi, 8th edition, 1995, CAB International, University Press, Cambridge, UK).

The fungal host cell may be a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, Passmore, and Davenport, editors, Soc. App. Bacteriol. Symposium Series No. 9, 1980).

The yeast host cell may be a Candida, Hansenula, Kluyveromyces, Pichia, Saccharomyces, Schizosaccharomyces, or Yarrowia cell such as a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell.

The fungal host cell may be a filamentous fungal cell. “Filamentous fungi” include all filamentous forms of the subdivision Eumycota and Oomycota (as defined by Hawksworth et al., 1995, supra).

The filamentous fungi are generally characterized by a mycelial wall composed of chitin, cellulose, glucan, chitosan, mannan, and other complex polysaccharides. Vegetative growth is by hyphal elongation and carbon catabolismis obligately aerobic. In contrast, vegetative growth by yeasts such as Saccharomyces cerevisiae is by budding of a unicellular thallus and carbon catabolism may be fermentative.

The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

For example, the filamentous fungal host cell may be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride cell.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238023, Yelton et al., 1984, Proc. Natl. Acad. Sci. USA 81: 1470-1474, and Christensen et al., 1988, Bio/Technology 6: 1419-1422. Suitable methods for transforming Fusarium species are described by Malardier et al., 1989, Gene 78: 147-156, and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, J. Bacteriol. 153: 163; and Hinnen et al., 1978, Proc. Natl. Acad. Sci. USA 75:1920.

### Methods of Production

The present invention also relates to methods of producing a lactase variant of the present invention, comprising (a) cultivating a cell, under conditions conducive for production of the lactase variants; and optionally, (b) recovering the lactase variant. In some aspects, the cell is a Bifdidobacterium cell. In another aspect, the cell is a Bifidobacterium bifidum cell.

The present invention also relates to methods of producing a lactase variant of the present invention, comprising (a) cultivating a recombinant host cell of the present invention under conditions conducive for production of the lactase variant; and optionally, (b) recovering the lactase variant.

The recombinant host cells are cultivated in a nutrient medium suitable for production of the variant using methods known in the art. For example, the cells may be cultivated by shake flask cultivation, or small-scale or large-scale fermentation (including continuous, batch, fed-batch, or solid state fermentations) in laboratory or industrial fermentors in a suitable medium and under conditions allowing the variant to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the variant is secreted into the nutrient medium, the variant can be recovered directly from the medium. If the variant is not secreted, it can be recovered from cell lysates.

The variants may be detected using methods known in the art that are specific for the variants. These detection methods include, but are not limited to, use of specific antibodies, formation of an enzyme product, or disappearance of an enzyme substrate. For example, an enzyme assay may be used to determine the activity of the variant.

The variant may be recovered using methods known in the art. For example, the variant may be recovered from the nutrient medium by conventional procedures including, but not limited to, collection, centrifugation, filtration, extraction, spray-drying, evaporation, or precipitation. In one aspect, the whole fermentation broth is recovered.

The variant may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, Janson and Ryden, editors, VCH Publishers, New York, 1989) to obtain substantially pure variants.

In an alternative aspect, the variant is not recovered, but rather a host cell of the present invention expressing the variant is used as a source of the variant.

### Enzyme Compositions

The present invention also relates to a liquid composition comprising a variant of the invention and 20-70% glycerol.

The present invention also relates to compositions comprising a variant of the present invention. Preferably, the compositions are enriched in such a variant. The term “enriched” indicates that the lactase activity of the composition has been increased, e.g., with an enrichment factor of at least 1.1.

The compositions may comprise a variant of the present invention as the major enzymatic component, e.g., a mono-component composition. Alternatively, the compositions may comprise multiple enzymatic activities, such as one or more enzymes selected from the group consisting of hydrolase, isomerase, ligase, lyase, oxidoreductase, or transferase, e.g., an alpha-galactosidase, alpha-glucosidase, aminopeptidase, amylase, beta-galactosidase, beta-glucosidase, beta-xylosidase, carbohydrase, carboxypeptidase, catalase, cellobiohydrolase, cellulase, chitinase, cutinase, cyclodextrin glycosyltransferase, deoxyribonuclease, endoglucanase, esterase, glucoamylase, invertase, laccase, lipase, mannosidase, mutanase, oxidase, pectinolytic enzyme, peroxidase, phytase, polyphenoloxidase, proteolytic enzyme, ribonuclease, transglutaminase, or xylanase.

The compositions may be prepared in accordance with methods known in the art and may be in the form of a liquid or a dry composition. The compositions may be stabilized in accordance with methods known in the art.

### Method for Obtaining a Lactase Variant

In one aspect, the present invention relates to a method for obtaining a lactase variant, comprising:
a. introducing into a parent lactase, preferably a parent lactase having an amino acid sequence which is at least 80%, 85%, 90%, or 95%, preferably at least 97%, 98%, or 99%, more preferably at least 99.5%, 99.7%, 99.8%, 99.9%, or 100%, identical to SEQ ID NO: 1, an alteration, preferably a substitution, at one or more positions corresponding to positions 1, 2, 3, 4, 5, 6, 7, 9, 10, 11, 12, 13, 14, 15, 16, 17, 19, 20, 21, 22, 23, 24, 25, 26, 27, 28, 29, 30, 31, 32, 33, 34, 35, 36, 37, 38, 39, 40, 41, 42, 43, 44, 45, 47, 48, 49, 51, 53, 54, 55, 57, 58, 59, 60, 61, 62, 63, 64, 66, 67, 68, 70, 71, 73, 74, 75, 76, 77, 78, 79, 80, 81, 83, 85, 86, 87, 88, 89, 90, 91, 92, 93, 94, 95, 96, 97, 98, 100, 101, 102, 104, 105, 106, 107, 108, 109, 110, 111, 112, 113, 114, 115, 116, 117, 118, 119, 120, 121, 122, 123, 124, 125, 126, 128, 129, 130, 131, 132, 133, 135, 136, 137, 138, 139, 140, 141, 142, 146, 148, 149, 150, 151, 153, 154, 155, 157, 158, 159, 160, 161, 162, 164, 165, 166, 167, 168, 169, 170, 171, 172, 173, 174, 175, 176, 177, 178, 179, 180, 181, 182, 183, 184, 185, 186, 187, 188, 189, 190, 191, 193, 194, 195, 196, 197, 198, 199, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 254, 255, 256, 257, 258, 259, 260, 261, 262, 263, 264, 265, 266, 267, 268, 269, 270, 271, 272, 273, 274, 275, 277, 278, 279, 280, 281, 282, 283, 284, 285, 286, 287, 288, 291, 292, 293, 294, 295, 296, 297, 298, 299, 301, 302, 303, 304, 305, 306, 307, 308, 309, 310, 311, 312, 314, 315, 316, 317, 318, 320, 321, 322, 324, 325, 326, 328, 329, 331, 335, 337, 338, 340, 341, 342, 343, 344, 345, 346, 347, 348, 349, 350, 351, 352, 353, 354, 355, 357, 359, 360, 362, 363, 364, 365, 366, 367, 368, 369, 370, 371, 372, 373, 374, 375, 376, 377, 378, 379, 380, 381, 383, 386, 387, 388, 389, 390, 391, 392, 393, 394, 395, 396, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 485, 487, 488, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 534, 535, 537, 538, 539, 540, 542, 543, 545, 546, 547, 548, 549, 550, 551, 553, 554, 555, 556, 557, 558, 559, 560, 561, 563, 564, 565, 567, 570, 571, 572, 573, 574, 575, 576, 577, 578, 579, 580, 581, 582, 583, 584, 585, 586, 587, 588, 589, 590, 591, 592, 593, 594, 595, 597, 598, 600, 604, 605, 606, 607, 608, 609, 610, 611, 612, 613, 614, 615, 616, 619, 620, 621, 624, 625, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 659, 661, 662, 667, 669, 670, 672, 673, 674, 675, 676, 677, 678, 679, 680, 681, 682, 683, 684, 685, 686, 687, 688, 689, 690, 691, 692, 693, 694, 695, 696, 697, 698, 700, 701, 703, 704, 705, 706, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 765, 766, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 857, 858, 859, 860, 861, 862, 863, 864, 865, 866, 867, 870, 871, 872, 873, 874, 875, 877, 879, 880, 881, 882, 883, 884, 885, 886, 887, 888, 889, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 973, 974, 975, 976, 977, 978, 979, 980, 981, 982, 984, 985, 986, 987, 988, 989, 990, 991, 992, 993, 994, 995, 996, 997, 998, 999, 1000, 1001, 1002, 1003, 1004, 1005, 1006, 1007, 1008, 1009, 1010, 1011, 1012, 1013, 1014, 1015, 1016, 1017, 1018, 1021, 1022, 1023, 1024, 1026, 1027, 1028, 1029, 1030, 1031, 1033, 1034, 1035, 1036, 1037, 1038, 1039, 1040, 1041, 1042, 1043, 1044, 1045, 1046, 1047, 1048, 1050, 1051, 1052, 1053, 1054, 1055, 1057, 1058, 1059, 1060, 1061, 1062, 1063, 1064, 1065, 1066, 1067, 1068, 1069, 1070, 1071, 1072, 1073, 1074, 1075, 1076, 1077, 1078, 1079, 1081, 1082, 1083, 1084, 1085, 1086, 1087, 1088, 1089, 1090, 1091, 1092, 1093, 1094, 1095, 1096, 1097, 1098, 1099, 1100, 1101, 1102, 1103, 1104, 1105, 1106, 1107, 1108, 1109, 1111, 1113, 1114, 1115, 1116, 1117, 1118, 1119, 1120, 1121, 1122, 1123, 1124, 1125, 1126, 1127, 1128, 1129, 1130, 1131, 1132, 1133, 1134, 1135, 1136, 1137, 1138, 1139, 1140, 1141, 1142, 1143, 1144, 1145, 1146, 1147, 1148, 1149, 1150, 1151, 1152, 1153, 1154, 1155, 1157, 1158, 1159, 1160, 1161, 1162, 1165, 1166, 1167, 1168, 1169, 1170, 1171, 1172, 1173, 1174, 1175, 1177, 1178, 1179, 1180, 1181, 1182, 1183, 1184, 1185, 1186, 1188, 1189, 1190, 1191, 1192, 1193, 1194, 1195, 1197, 1198, 1199, 1200, 1202, 1203, 1204, 1205, 1208, 1209, 1210, 1211, 1213, 1214, 1215, 1216, 1217, 1218, 1219, 1220, 1221, 1222, 1223, 1224, 1225, 1226, 1227, 1228, 1229, 1230, 1231, 1232, 1233, 1234, 1235, 1236, 1237, 1238, 1239, 1240, 1241, 1242, 1243, 1244, 1246, 1247, 1248, 1249, 1250, 1251, 1252, 1253, 1254, 1255, 1256, 1257, 1258, 1259, 1261, 1262, 1263, 1264, 1265, 1266, 1267, 1269, 1270, 1271, 1272, 1273, 1274, 1275, 1276, 1277, 1278, 1279, 1280, 1281, 1282, 1283, 1284, 1285, 1286, 1287, 1288, 1289, 1290, 1291, 1292, 1293, 1294, 1295, 1296, 1297, 1298, 1301, 1302, 1303 or 1304 of the polypeptide of SEQ ID NO: 1, and
b. recovering the variant.

In preferred embodiments, an alteration, preferably a substitution, is introduced at one or more positions corresponding to the positions listed in any one of embodiments 3, 5, 7, 9, 11, 13, 15, 17 19, 21, 23, 25, 27, 29, 31, 33, 35, 37, 39, 41, 43, 45, 47, 49, 51, 53, 55, 57, 59, 61, 63, 65, 67, 69 or 71 of the “PREFERRED EMBODIMENTS” section right before the “EXAMPLES” section of the present application.

In a preferred aspect, the method for obtaining a lactase variant, comprises:
a. introducing into a parent lactase, preferably a parent lactase having an amino acid sequence which is at least 80%, 85%, 90%, or 95%, preferably at least 97%, 98%, or 99%, more preferably at least 99.5%, 99.7%, 99.8%, 99.9%, or 100%, identical to SEQ ID NO: 1, one or more of the following substitutions, wherein each position corresponds to a position in SEQ ID NO: 1: V1D, V1F, V1H, V1K, V1L, V1R, E2D, E2G, E2Q, E2V, D3A, D3H, D3I, D3N, D3S, D3V, D3W, A4C, A4G, A4H, A4I, A4L, A4M, A4P, T5A, T5D, T5F, T5K, T5R, T5S, T5V, R6A, R6D, R6F, R6G, R6H, R6L, R6M, R6P, R6S, R6W, S7A, S7D, S7I, S7L, S7N, S7P, S7T, S7W, S9G, S9H, S9P, S9R, S9W, T10G, T10K, T10L, T10P, T10R, T10S, T10W, T11L, T11P, Q12L, Q12R, Q12V, Q12Y, M13C, M13D, M13E, M13F, M13H, M13K, M13R, M13W, S14A, S14G, S14H, S14L, S14T, S14V, S14Y, S15C, S15F, S15I, S15K, S15L, S15P, S15R, S15T, S15V, S15W, S15Y, T16A, T16C, T16I, T16N, T16S, P17A, P17C, P17D, P17E, P17I, P17L, P17N, P17R, P17S, P17T, V19A, V19F, V19G, V19I, V19K, V19L, V19N, V19S, V19W, V20C, V20F, V20G, V20I, V20K, V20L, V20M, V20N, V20P, V20Q, V20R, V20T, V20W, Y21A, Y21C, Y21D, Y21F, Y21G, Y21H, Y21M, Y21P, Y21R, Y21T, S22A, S22E, S22F, S22G, S22L, S22M, S22N, S22R, S22T, S22W, S23A, S23C, S23D, S23L, S23M, S23R, A24F, A24G, A24L, A24R, A24T, A24W, V25D, V25E, V25F, V25G, V25H, V25K, V25L, V25M, V25Q, V25R, V25S, V25T, V25W, D26C, D26I, D26L, D26M, D26T, D26V, S27A, S27C, S27F, S27G, S27H, S27P, S27Y, K28C, K28G, K28I, K28L, K28R, K28S, K28V, K28W, Q29D, Q29F, Q29G, Q29L, Q29M, Q29R, Q29S, Q29V, Q29W, N30A, N30G, N30H, N30M, N30P, N30V, N30W, N30Y, R31E, R31G, R31I, R31M, R31V, T32M, T32Q, T32R, T32S, S33C, S33E, S33H, S33K, S33N, S33Q, S33R, S33V, D34C, D34E, D34F, D34G, D34H, D34L, D34S, D34W, D34Y, F35A, F35C, F35E, F35G, F35K, F35N, F35T, F35V, D36H, D36Q, A37N, A37Q, N38C, N38G, N38S, W39G, W39S, K40C, K40D, K40F, K40G, K40I, K40M, K40N, K40P, K40W, F41A, F41C, F41G, F41I, F41Q, F41S, F41Y, M42E, M42N, M42T, L43A, L43C, L43G, L43I, L43S, L43T, L43V, S44C, S44M, S44N, S44Y, D45A, D45L, D45P, D45V, V47K, V47R, Q48S, A49C, A49D, A49H, A49R, A49S, A49T, A49V, D51G, D51I, D51K, D51M, D51P, D51V, A53C, A53G, A53L, A53R, A53S, A53T, A53V, A53W, F54M, F54S, D55C, D55F, D55G, D55H, D55M, D55N, D55P, D55S, D55V, S57A, S57C, S57E, S57G, A58D, A58G, A58I, A58M, A58N, A58Q, A58R, A58T, W59D, W59I, W59K, W59L, W59N, W59P, W59V, Q60A, Q60C, Q60E, Q60F, Q60G, Q60K, Q60L, Q60M, Q60R, Q60S, Q60V, Q60Y, Q61K, Q61P, Q61S, V62G, V62N, V62S, V62T, V62W, D63G, D63L, D63P, D63S, D63V, L64E, L64G, H66C, H66L, H66R, H66T, H66W, H66Y, D67E, Y68P, Y68V, I70A, I70H, I70K, I70P, I70R, T71C, T71E, T71G, T71H, T71K, T71L, T71N, T71P, T71Q, T71R, T71S, K73A, K73D, K73F, K73G, K73Q, K73V, Y74G, Y74K, Y74T, Y74W, S75G, S75L, S75Q, S75R, S75V, Q76G, Q761, Q76K, Q76M, Q76P, Q76S, Q76V, Q76Y, S77C, S77D, S77E, S77F, S77G, S77H, S77I, S77K, S77L, S77M, S77R, S77T, S77V, S77W, S77Y, N78C, N78E, N78F, N78K, N78Q, N78R, N78S, N78T, E79H, E79Q, E79S, E79T, E79W, A80K, E81A, E81Q, A83E, A83T, L85A, L85C, L85D, L85F, L85M, L85N, L85S, L85V, L85W, P86E, P86G, P86H, P86N, P86Q, P86R, P86V, P86W, P86Y, G87A, G87D, G87E, G87N, G87Q, G88A, G88F, G88I, G88M, G88Q, G88S, T89C, T89G, T89H, T89K, T89L, T89M, T89N, T89P, T89W, T89Y, G90A, G90C, G90D, G90L, G90S, G90T, G90V, W91E, W91L, W91P, W91Q, W91R, W91S, W91Y, Y92F, Y92H, Y92I, Y92M, Y92N, Y92S, Y92T, Y92V, Y92W, R93A, R93D, R93F, R93H, R93I, R93L, R93N, R93T, R93V, R93Y, K94A, K94C, K94G, K94R, K94S, K94T, K94V, S95A, S95C, S95D, S95E, S95G, S95I, S95L, S95Q, S95R, F96A, F96C, F96I, F96K, F96L, F96M, F96P, F96S, F96V, F96W, T97F, T97S, T97V, I98C, I98H, I98S, I98W, R100T, D101A, D101P, D101V, L102A, L102G, L102M, L102P, L102S, G104C, K105D, K105Q, K105R, K105W, K105Y, R106K, R106P, R106V, R106W, I107A, I107F, I107G, I107Q, I107S, A108E, A108S, A108V, I109M, I109T, N110A, N110F, N110S, N110T, N110V, N110W, F111A, F111C, F111L, F111Q, F111V, D112A, D112F, D112G, D112T, G113A, G113S, V114F, V114G, V114M, V114R, Y115E, M116A, M116C, M116D, M116W, M116Y, N117K, N117R, N117T, N117W, A118K, A118P, A118Y, T119A, T119G, T119L, V120A, V120K, W121C, W121D, W121R, W121T, W121V, W121Y, F122A, F122M, F122S, F122Y, N123P, G124E, G124M, G124Q, G124R, V125D, V125E, V125I, K126E, K126V, G128A, G128D, T129E, T129V, H130A, H130C, H130Q, H130S, H130T, P131K, P131L, P131S, Y132C, Y132E, Y132S, G133E, S135E, S135P, S135V, P136R, P136Y, F137A, F137C, F137D, F137G, F137L, F137P, S138A, S138D, S138G, S138H, S138L, S138M, S138R, S138V, F139A, F139E, F139Q, F139W, D140G, D140L, D140V, L141G, L141T, T142E, T142S, T142V, K146A, G148H, G148K, G148T, G149E, G149H, G149I, G149M, G149Q, G149Y, E150A, E150C, E150G, E150L, E150N, E150R, N151L, I153A, I153Y, V154E, V154I, V154K, V154L, V154M, V154S, V155F, V157A, V157G, V157L, V157P, V157Q, V157S, E158G, E158H, E158K, E158Q, E158V, N159D, N159H, N159S, N159T, R160G, L161E, L161K, L161M, L161S, L161W, P162F, P162G, P162N, P162T, P162W, S164A, R165H, W166S, Y167A, Y167C, S168C, G169A, G169C, G169D, G169S, S170L, S170Q, G171C, G171F, G171T, I172G, I172K, I172P, I172Q, Y173A, Y173H, Y173M, Y173P, Y173S, Y173W, R174E, R174K, D175E, D175Y, V176E, V176K, V176T, T177A, T177C, T177E, T177K, T177L, L178I, L178Q, L178W, T179A, T179C, T179D, T179H, T179I, T179K, T179L, T179N, T179P, T179S, V180A, V180C, V180D, V180E, V180G, V180M, T181A, T181D, T181F, T181K, T181R, D182F, D182L, D182S, G183W, V184F, V184H, V184P, V184Q, V184R, V184S, V184W, H185G, H185L, H185R, V186A, V186E, V186G, V186N, G187A, G187D, G187H, N188E, N188R, N188S, N188V, N188W, N189A, N189E, G190C, G190F, G190H, G190Q, G190V, V191C, V191T, V191W, V191Y, I193N, I193Q, I193T, I193V, K194A, K194I, K194L, K194R, T195A, T195E, T195M, T195S, T195W, P196A, P196H, P196I, P196M, P196S, P196W, S197A, S197C, S197E, S197K, S197L, S197P, L198E, L198F, L198H, L198I, L198K, L198R, L198V, L198W, A199E, A199F, A199K, A199P, A199R, A199T, Q201C, Q201E, Q201I, Q201K, Q201M, Q201V, N202A, N202D, N202F, N202G, N202K, N202L, N202M, N202Q, N202R, N202S, N202T, N202W, G203C, G203K, G203M, G203Q, G203R, G203S, G203V, G203W, G203Y, G204A, G204C, G204D, G204K, G204R, G204S, G204Y, N205E, N205G, N205H, N205L, N205P, N205W, N205Y, V206A, V206C, V206D, V206F, V206G, V206I, V206K, V206Q, V206R, V206S, V206T, T207A, T207C, T207G, T207I, T207K, T207L, T207M, T207N, T207Q, T207R, T207W, M208A, M208S, M208T, N209C, N209D, N209G, N209K, N209L, N209Q, N209R, N209V, L210A, L210C, L210F, L210G, L210H, L210I, L210Q, L210R, L210S, L210T, L210V, T211A, T211D, T211E, T211F, T211K, T211N, T211Q, T211R, T211S, T212A, T212C, T212E, T212F, T212G, T212H, T212K, T212L, T212M, T212S, T212W, K213A, K213C, K213D, K213F, K213I, K213L, K213M, K213N, K213Q, K213R, K213S, K213T, K213V, K213Y, V214A, V214C, V214T, V214W, A215D, A215E, A215F, A215I, A215K, A215L, A215Q, A215R, A215S, A215V, N216D, N216T, N216V, D217F, D217G, D217L, D217M, D217T, D217V, T218D, T218G, T218H, K219A, K219C, K219F, K219H, K219M, A220C, A220G, A220I, A220L, A220M, A220T, A220V, A220W, A221C, A221D, A221E, A221L, A221N, A221R, A221V, A221Y, A222D, A222I, A222L, A222P, A222R, A222W, A222Y, N223A, N223E, N223F, N223G, N223K, N223L, N223M, N223R, N223S, N223T, N223V, N223W, I224G, I224Q, T225A, T225G, T225L, L226C, L226M, L226Q, K227T, Q228N, Q228R, T229A, T229C, T229D, T229G, T229H, T229M, T229N, T229Q, T229R, T229V, V230F, V230L, V230M, V230Q, V230R, V230S, F231A, F231E, F231G, F231I, F231K, F231L, F231Q, F231S, F231V, F231W, F231Y, P232G, P232H, P232L, P232M, P232R, P232S, P232T, P232V, P232W, P232Y, K233A, K233C, K233E, K233F, K233G, K233L, K233P, K233R, K233S, K233V, K233W, K233Y, G234A, G234C, G234D, G234E, G234K, G234L, G234Q, G234R, G234V, G234W, G234Y, G235C, G235F, G235H, G235l, G235K, G235M, G235Q, G235R, G235T, G235W, G235Y, K236A, K236D, K236E, K236G, K236L, K236M, K236P, K236R, K236S, K236T, K236W, K236Y, T237D, T237F, T237I, T237K, T237M, T237Q, T237R, T237S, T237V, T237Y, D238A, D238E, D238F, D238G, D238H, D238I, D238K, D238L, D238M, D238N, D238P, D238Q, D238R, A239C, A239E, A239G, A239I, A239K, A239T, A240C, A240E, A240L, A240P, A240Q, A240T, A240V, A240W, A240Y, I241T, G242K, G242L, G242M, G242P, G242T, G242Y, T243I, T243M, T243R, T243V, V244A, V244E, V244G, V244L, V244R, T245E, T245G, T245L, T245M, T245N, T245Q, T245R, T245S, T246D, T246E, T246G, T246K, T246V, A247D, A247E, A247K, A247N, A247P, A247Q, A247R, A247S, A247V, A247W, S248A, S248E, S248F, S248H, S248I, S248L, S248Q, S248T, S248Y, K249A, K249D, K249G, K249H, K249I, K249L, K249M, K249N, K249P, K249Q, K249S, K249T, K249V, K249Y, S250H, S250M, S250W, I251F, I251L, I251V, I251W, I251Y, A252C, A252E, A252F, A252H, A252I, A252P, A252R, A252S, A252W, A252Y, G254D, G254F, G254I, G254L, G254M, G254Q, G254R, G254W, A255C, A255F, A255K, A255L, A255M, A255S, A255T, A255W, A255Y, S256A, S256C, S256F, S256G, S256K, S256L, S256M, S256N, S256Q, S256R, S256V, S256W, S256Y, A257D, A257G, A257I, A257N, A257T, A257V, D258A, D258L, D258M, D258W, V259E, V259L, V259S, V259T, T260A, T260D, T260G, T260I, T260K, T260V, S261A, S261D, S261H, S261R, S261W, S261Y, T262D, T262E, T262F, T262G, T262H, T262L, T262P, T262W, I263A, I263C, I263G, I263L, I263S, I263V, T264F, T264G, T264K, T264L, T264M, T264P, T264Q, T264R, T264S, T264Y, A265G, A265I, A265K, A265R, A265S, A265V, A266D, A266E, A266G, A266K, A266L, A266M, A266P, A266Q, A266S, A266T, S267A, S267D, S267K, S267M, S267N, S267P, S267Q, S267R, S267V, P268F, P268G, P268M, P268R, P268V, P268W, P268Y, K269G, K269R, K269V, K269Y, L270D, L270M, L270N, L270R, L270V, W271T, S272E, S272G, S272K, S272L, S272N, S272T, S272W, I273K, I273L, I273P, I273R, I273S, I273W, K274D, K274P, K274Q, K274R, N275K, N275M, N275V, N275W, N277F, N277K, N277R, L278A, L278G, L278H, L278I, L278K, L278M, L278P, L278Q, L278R, L278S, L278V, Y279M, Y279T, Y279W, T280A, T280D, T280E, T280F, T280H, T280M, T280Q, V281A, V281I, V281L, V281Q, R282E, R282F, R282H, R282I, R282K, R282N, R282S, R282T, R282V, R282W, T283M, T283R, T283V, E284A, E284D, E284F, E284H, E284L, E284M, E284N, E284Q, E284R, E284Y, V285H, V285I, V285T, L286A, L286C, L286D, L286F, L286N, L286R, L286T, L286W, L286Y, N287I, N287L, G288F, G288L, G288S, V291C, V291D, V291F, V291G, V291H, V291L, V291P, V291S, V291T, V291Y, L292A, L292D, L292E, L292H, L292Q, L292S, L292V, D293C, D293E, D293F, D293I, D293S, D293V, D293W, T294C, T294G, T294M, T294Q, T294S, Y295F, Y295G, Y295I, Y295L, Y295R, Y295S, Y295W, D296F, D296H, D296K, D296L, D296N, D296R, D296V, T297G, T297I, T297Q, T297R, T297S, E298G, E298I, E298L, E298M, E298R, E298T, Y299F, Y299S, F301C, F301L, R302E, R302I, R302K, R302M, R302N, R302T, W303A, W303C, W303D, W303F, W303S, W303T, T304D, T304E, T304I, T304K, T304P, T304S, T304W, G305E, G305F, G305L, G305M, G305N, G305P, G305T, G305W, F306D, F306K, F306L, F306S, D307A, D307E, D307F, D307G, D307L, D307M, D307N, D307Q, D307S, D307V, D307W, A308C, A308E, A308G, A308I, A308M, A308Y, T309C, T309D, T309E, T309I, T309K, T309M, T309S, T309V, S310A, S310C, S310G, S310H, S310I, S310L, S310M, S310N, S310R, S310V, G311E, G311F, G311L, G311Q, G311R, G311V, F312H, F312L, F312R, F312V, L314A, L314C, L314T, L314V, L314Y, N315S, G316A, G316C, G316N, G316R, G316S, G316W, G316Y, E317D, E317H, E317I, E317K, E317V, E317W, E317Y, K318C, K318E, K318F, K318G, K318H, K318L, K318M, K318N, K318P, K318Q, K318V, K320C, K320E, K320F, K320I, K320L, K320R, K320T, K320V, K320W, L321C, L321G, L321H, L321M, L321V, L321Y, K322C, K322F, K322I, K322N, K322P, K322Q, K322R, K322S, V324E, V324G, V324T, S325D, S325G, S325T, M326E, M326G, M326S, M326T, M326V, M326Y, H328C, H328D, H328F, H328G, H3281, H328L, H328M, H328R, H328T, D329S, D329T, G331V, A335C, A335G, A335L, A337D, A337E, A337G, A337L, A337Q, N338P, N338R, N338V, R340A, R340C, R340L, A341M, A341S, I342A, I342G, I342K, I342P, E343A, E343H, E343N, E343R, E343S, E343T, E343Y, R344G, R344L, R344Y, Q345F, Q345G, Q345K, Q345N, Q345S, V346A, V346C, V346F, V346I, V346L, V346S, E347A, E347C, E347D, E347F, E347I, E347R, E347S, I348A, I348D, I348G, I348M, I348Q, I348R, L349A, L349E, L349Q, L349S, L349V, Q350G, Q350N, Q350W, K351N, K351R, K351V, K351W, M352G, M352L, M352T, M352W, G353K, V354E, V354G, V354S, V354W, N355H, N355M, N355R, N355W, I357T, T359E, T359L, T360V, N362G, N362S, N362T, P363A, P363D, P363I, P363L, P363M, P363S, P363V, A364C, A364E, A364F, A364G, A364I, A364M, A364P, A364V, A364W, A365C, A365E, A365I, A365P, A365V, A365W, K366A, K366D, K366E, K366I, K366L, K366M, K366P, K366S, K366V, A367C, A367I, A367N, A367Q, L368A, L368E, L368Q, L368S, L368V, L368Y, I369A, I369D, I369E, I369G, I369K, I369M, I369R, I369V, I369W, D370C, D370L, D370Q, D370R, D370S, D370T, V371D, V371F, V371G, V371I, V371L, V371Q, V371S, C372P, N373G, N373L, N373R, E374K, E374L, E374R, K375D, K375I, K375N, K375Q, K375S, G376A, G376S, V377A, V377M, V377T, L378I, L378P, L378W, L378Y, V379A, V379C, V379M, V379N, V380M, V380P, V380S, E381A, E381C, E381G, E381L, E381Q, E381T, V383A, V383K, V383L, M386G, M386N, M386Q, M386S, M386V, W387H, W387L, N388A, N388E, N388L, N388R, R389A, R389C, R389E, R389K, R389M, R389N, R389Q, R389S, R389T, R389V, S390C, S390D, S390G, S390H, S390N, S390P, S390Q, S390T, S390V, K391E, N392D, G393A, G393E, G393N, G393R, G393S, G393V, N394L, T395A, T395C, T395F, T395H, T395I, T395M, T395N, T395Q, T395S, T395W, E396K, E396L, E396M, E396V, E396W, Y398M, Y398N, G399S, K400A, K400C, K400D, K400E, K400M, K400N, K400P, K400Q, K400S, K400T, K400V, W401F, W401H, W401K, W401L, W401M, W401R, F402T, F402W, F402Y, G403A, G403D, G403H, G403K, G403P, G403Q, G403S, G403T, G403V, G403Y, Q404F, Q404H, Q404L, Q404M, Q404P, Q404R, Q404S, Q404V, A405C, A405E, A405H, A405K, A405P, A405R, A405T, A405V, I406C, I406D, I406N, A407C, A407G, A407M, A407Q, A407S, A407T, A407W, G408D, G408I, G408M, G408N, G408W, D409N, D409W, N410C, N410R, N410Y, A411E, A411N, A411R, A411S, A411V, V412M, V412S, L413D, L413E, L413F, L413I, L413P, L413T, G414A, G414C, G414M, G414N, G414R, G414T, G414W, G415A, G415Q, G415R, D416I, D416M, D416R, D416T, D416Y, K417C, K417F, K417G, K417R, K417T, D418L, D418P, D418R, D418Y, E419M, E419R, E419W, T420E, T420F, T420G, T420K, T420R, T420V, W421L, W421Q, W421S, A422P, A422T, A422V, K423D, K423L, K423M, K423R, F424C, F424L, F424N, F424T, D425E, L426C, L426M, L426Q, T427D, T427F, T427G, T427K, T427M, T427P, T427Q, T427R, T427S, T427W, S428F, S428K, S428W, T429D, T429P, I430C, I430D, I430E, I430L, I430M, I430Q, I430S, I430T, I430W, N431D, N431E, N431G, N431L, N431M, N431R, N431V, N431Y, R432A, R432E, R432F, R432G, R432N, R432Q, R432V, R432Y, D433C, D433G, D433H, D433I, D433P, D433Q, D433W, R434L, R434M, R434N, R434P, R434S, R434T, R434V, N435E, N435F, N435H, N435K, N435L, N435M, N435R, N435V, N435W, A436C, A436D, A436E, A436G, A436I, A436L, A436M, A436Q, A436S, P437A, P437D, P437K, P437L, P437Q, P437R, P437S, P437V, P437W, S438G, V439C, V439E, V439G, V439I, V439K, V439Q, V439T, V439Y, I440C, I440D, I440F, I440K, I440P, I440R, I440S, I440T, I440V, I440W, M441A, M441E, M441G, M441Q, M441R, M441T, M441V, W442E, W442G, W442M, W442P, W442Q, W442R, S443C, S443D, S443G, S443M, S443Q, S443Y, L444C, L444D, L444E, L444F, L444G, L444H, L444K, L444Q, L444V, L444W, G445A, G445C, G445V, N446D, N446T, M448A, M448C, M448D, M448E, M448I, M448L, M448P, M448Q, M448S, M448V, M448W, M449D, M449E, M449F, M449T, M449V, M449W, M449Y, E450F, E450S, E450T, E450V, E450W, G451C, G451F, G451L, G451P, G451V, G451W, I452G, I452K, I452L, I452M, I452Q, I452S, I452V, S453C, S453F, S453G, S453H, S453L, S453M, S453N, S453P, S453Q, S453R, S453V, G454L, G454W, S455A, S455E, S455K, S455M, S455P, S455R, S455V, S455W, V456A, V456D, V456E, V456F, V456K, V456L, V456W, S457E, S457H, S457K, S457L, S457M, S457P, S457Q, S457T, S457V, G458A, G458C, G458D, G458F, G458L, G458P, G458Q, G458S, G458V, G458W, F459A, F459C, F459E, F459G, F459N, F459R, F459S, F459T, F459W, P460C, P460M, P460Q, P460W, P460Y, A461D, A461G, A461M, A461N, A461Q, A461S, A461V, A461Y, T462C, T462E, T462F, T462L, T462M, T462S, S463G, S463K, S463Q, S463R, S463T, S463V, A464E, A464H, A464L, A464M, A464P, A464V, A464W, K465C, K465F, K465G, K465L, K465Q, K465R, K465V, K465W, K465Y, L466A, L466C, L466D, L466E, L466F, L466G, L466M, L466P, L466Q, L466S, L466V, L466Y, V467A, V467C, V467D, V467E, V467G, V467T, V467W, A468D, A468E, A468F, A468K, A468L, A468P, A468S, A468V, A468W, W469A, W469C, W469D, W469G, W469L, W469M, W469R, W469V, W469Y, T470E, T470L, T470M, T470Q, K471F, K471G, K471Q, K471W, K471Y, A472G, A472Y, A473E, A473M, A473P, D474A, D474C, D474E, D474K, D474M, D474R, D474W, S475E, S475F, S475Q, S475T, S475V, T476C, T476L, T476S, R477A, R477C, R477G, R477L, P478A, P478D, P478G, P478L, P478V, M479G, M479I, M479R, M479W, T480C, T480G, T480Q, K485E, K485R, K487A, K487C, K487F, K487G, K487N, K487S, K487W, A488C, A488G, A488H, A488L, A488N, A488S, A488V, N491A, N491E, N491W, E492A, E492W, S493E, S493G, S493H, S493L, S493M, S493Q, S493V, N494A, N494I, N494M, N494R, N494V, T495K, T495R, T495V, T495W, M496A, M496F, M496T, G497D, D498A, D498C, D498M, D498S, N499K, N499R, N499T, N499Y, L500A, L500E, L500N, L500V, T501C, T501G, T501M, A502L, A502Q, N503A, N503E, N503M, N503S, G504H, G504K, G504P, G505A, G505D, G505E, G505H, G505L, G505N, G505R, G505S, G505V, V506C, V506D, V506E, V506G, V506I, V506L, V506P, V506R, V506S, V506T, V506W, V507A, V507F, V507G, V507L, V507N, V507P, V507R, V507S, V507T, G508C, G508E, T509A, T509D, T509E, T509I, T509K, T509M, T509Q, T509S, T509V, T509Y, N510A, N510F, N510I, N510Q, Y511A, Y511K, S512C, S512E, S512F, S512G, S512I, S512M, S512Q, S512T, S512V, S512Y, D513C, D513G, D513K, D513L, D513M, D513P, D513Q, D513R, D513W, G514F, G514L, G514N, G514P, G514R, A515C, A515D, A515E, A515F, A515G, A515K, A515P, A515R, A515S, N516C, N516E, N516G, N516I, N516M, N516Q, N516S, N516T, N516V, Y517G, Y517I, Y517N, D518Q, D518Y, K519C, K519E, K519F, K519G, K519I, K519L, K519M, K519N, K519Q, K519S, K519T, I520A, I520C, I520G, I520H, I520M, I520N, I520Q, I520S, I520T, I520W, I520Y, R521A, R521C, R521K, R521N, R521Q, R521V, T522G, T522I, T522N, T523A, T523M, H524R, H524V, P525D, P525G, P525R, P525T, P525V, S526G, S526I, S526W, W527A, W527E, W527G, W527H, W527N, W527R, W527S, W527V, A528C, A528E, A528G, A528I, A528L, I529F, I529G, I529L, I529Y, Y530A, Y530M, G531E, G531S, G531T, T534A, T534I, T534Q, A535E, A535G, A535I, A535M, A537D, A537M, A537P, A537S, I538H, I538M, N539G, N539W, S540E, S540G, S540M, G542E, G542Q, G542S, G542T, I543V, I543W, N545G, N545I, N545Q, N545S, R546A, R546C, R546L, R546P, R546S, T547A, T547D, T547H, T547K, T547N, T547S, T548D, T548E, T548F, T548K, T548L, T548P, T548W, G549D, G549F, G549P, G549W, G550Q, G550R, G550S, A551D, A551I, A551Q, S553C, S553F, S553N, S553P, S553R, S553T, S553V, S554F, S554N, S554R, S554T, S554V, S554W, D555E, D555P, D555S, K556A, K556C, K556R, K556W, Q557F, Q557R, Q557S, L558E, L558H, L5581, L558P, T559A, T559G, T559I, T559Q, T559V, T559Y, S560P, S560V, Y561R, N563R, N563S, S564A, S564C, S564F, S564W, A565C, A565D, A565E, A565F, A565G, A565I, A565L, A565M, A565R, A565S, A565T, A565V, A565W, G567N, G567Q, G567V, A570G, A570K, A570L, A570M, A570S, A570T, A570W, V571W, A572S, A572W, S573G, S573K, S573Y, S574K, S574Q, S574V, S574W, A575D, A575M, A575V, W576A, W576F, W576V, W576Y, Y577G, Y577I, Y577L, Y577R, D578E, D578M, D578N, D578T, V579E, V579G, V579L, V579T, V580A, V580D, V580E, V580K, V580L, V580S, Q581F, Q581G, Q581P, Q581R, Q581S, Q581T, Q581Y, R582A, R582D, R582G, R582I, R582L, R582Y, D583V, D583W, F584E, F584I, F584W, V585I, V585M, V585Q, A586C, A586D, A586H, A586K, G587A, G587C, T588C, T588D, T588G, T588I, T588L, T588M, T588P, Y589A, Y589I, Y589Q, Y589V, Y589W, V590A, V590H, V590I, W591F, T592C, T592L, T592Q, T592S, G593C, G593I, F594C, F594I, F594L, F594M, D595E, D595Q, D595S, L597C, L597D, L597E, L597T, G598N, P600A, P600E, P600G, P600S, N604E, N604S, G605R, T606S, G607A, G607Q, G607S, S608E, S608I, S608M, S608N, S608Q, S608T, S608V, G609A, G609H, G609L, G609N, G609R, G609S, A610D, A610F, A610M, A610T, V611K, G612N, G612T, G612V, S613A, W614A, W614F, W614P, P615L, P615S, P615V, S616A, S616W, N619A, N619I, N619L, N619S, S620G, Y621W, I624A, I624V, V625A, V625E, V625F, V625M, V625Y, T627F, T627G, T627K, T627Q, A628C, A628D, A628N, G629T, F630A, F630C, F630D, F630G, F630S, F630Y, P631A, P631D, P631G, P631H, P631S, P631V, P631Y, K632C, K632D, K632G, K632T, D633G, T634A, T634E, T634F, T634S, T634V, Y635R, Y636K, F637C, F637G, F637I, F637S, F637T, F637V, Y638A, Y638W, Q639D, Q639N, Q639R, S640C, S640D, Q641T, W642I, N643R, D644C, D644G, D644Y, D645S, D645V, V646C, V646L, V646N, V646R, V646S, H647G, H647V, T648C, L649V, H650E, H650F, H650R, I651F, I651T, I651V, L652C, L652D, L652V, L652W, P653Q, A654D, A654K, A654M, A654R, W655F, W655R, N656A, N656K, N656V, E657K, E657R, E657V, V659D, V659N, A661E, A661G, A661H, A661K, A661L, A661M, A661Q, A661W, K662H, K662S, K662V, K662W, K662Y, N667L, N667R, P669A, P669E, P669F, P669L, P669R, P669T, P669W, V670C, V672L, Y673E, Y673G, Y673I, Y673R, Y673S, Y673W, T674C, T674D, T674G, T674H, T674M, T674Q, D675A, D675E, D675P, D675Q, D675S, D675V, D675Y, A676C, A676E, A676G, A676K, A676L, A676P, A676S, A676W, A677E, A677G, A677L, A677R, A677T, A677V, A677Y, K678A, K678C, K678T, K678V, V679G, V679Q, V679S, V679T, V679Y, K680A, K680E, K680G, K680H, K680I, K680L, K680N, K680Q, K680S, K680V, K680W, L681E, L681F, L681G, L681M, L681S, L681T, Y682D, Y682E, Y682I, Y682M, Y682S, Y682V, Y682W, F683H, F683L, F683M, F683Q, F683R, F683W, T684A, T684D, T684G, T684L, T684R, T684S, T684V, P685A, P685E, P685I, P685L, P685W, K686A, K686E, K686I, K686M, K686T, K686V, G687A, G687K, G687N, G687P, G687Q, G687R, S688C, S688D, S688E, S688G, S688K, S688L, S688P, S688T, T689A, T689D, T689E, T689G, T689L, T689P, T689S, T689W, T689Y, E690D, E690M, E690P, E690T, E690V, K691E, K691H, K691N, K691P, K691R, K691S, R692G, R692H, R692I, R692L, R692P, R692S, R692T, R692V, R692W, L693A, L693M, L693P, I694L, I694W, G695C, G695K, G695L, G695R, G695W, E696A, E696L, E696R, K697A, K697E, K697G, K697R, K697V, K697W, S698C, S698D, S698E, S698I, S698L, S698M, S698P, S698Q, S698R, S698T, T700A, T700C, T700D, T700E, T700G, T700K, T700Y, K701A, K701D, K701E, K701G, K701H, K701L, K701M, K701P, K701S, K701W, T703E, T703I, T703K, T703W, T704M, T704R, T704Y, A705C, A705D, A705E, A705K, A705N, A705P, A705R, A705V, A705W, A706C, A706G, A706T, A706W, A706Y, Y708C, Y708F, Y708G, Y708K, Y708L, Y708P, Y708T, T709M, Y710C, Y710D, Y710E, Y710G, Y710M, Y710N, Y710T, Y710V, Y710W, Q711A, Q711D, Q711G, Q711L, Q711M, Q711T, Q711Y, V712G, V712M, V712P, V712Q, V712R, Y713A, Y713E, Y713G, Y713L, Y713Q, E714D, E714H, E714I, E714K, E714M, E714N, E714R, E714S, E714V, E714Y, G715K, A716C, A716G, A716L, A716M, A716P, A716R, A716T, A716V, D717C, D717G, D717L, D717S, K718A, K718L, K718Q, K718T, K718W, K718Y, D719E, D719L, D719P, D719S, D719T, D719V, S720A, S720M, S720R, S720Y, T721D, T721H, T721K, T721L, T721N, T721P, T721Q, T721S, T721V, T721W, T721Y, A722Q, A722T, A722V, H723G, H723P, K724A, K724G, K724R, N725A, N725D, N725I, M726A, M726D, M726E, M726K, M726Q, M726V, M726W, Y727L, Y727T, L728C, L728K, L728Q, L728S, L728T, L728W, T729A, T729E, T729M, W730A, N731A, N731E, N731Q, N731S, N731Y, V732G, V732P, V732R, V732W, P733A, P733R, P733W, W734D, W734G, W734R, W734V, A735Q, A735R, A735S, A735T, A735W, E736S, G737F, G737K, G737N, G737Y, T738G, T738L, T738S, I739E, I739K, I739M, I739W, I739Y, S740D, S740E, S740F, S740H, S740L, A741I, A741P, A741S, A741V, E742D, E742M, E742Q, E742V, A743C, A743E, A743H, A743I, A743L, Y744A, Y744E, Y744I, Y744K, Y744L, Y744R, D745C, D745F, D745N, D745R, E746A, E746C, E746K, E746T, N747E, N747F, N747G, N747P, N747R, N748S, R749H, R749M, R749S, R749T, L750G, L750M, L750P, L750Q, L750S, I751C, I751H, I751Q, I751S, P752A, P752C, P752H, P752L, P752S, P752V, P752Y, E753Q, G754H, G754I, G754P, G754R, S755C, S755I, S755T, T756E, T756N, T756P, T756Q, T756S, T756W, E757A, G758A, G758V, N759D, N759R, N759S, N759V, A760G, A760N, A760P, A760Q, S761A, S761K, S761Q, V762D, V762G, V762K, V762W, T765A, T765P, T765R, T765W, G766M, G766S, A768H, A769F, A769G, A769I, A769N, A769V, A769Y, K770H, L771A, L771I, K772A, K772C, K772P, K772V, K772W, A773C, A773G, A773H, A773M, A773R, A773S, A773V, D774A, D774R, A775L, A775V, A775W, D776I, D776L, D776R, D776S, D776V, R777D, R777E, R777G, R777H, R777P, R777S, R777T, K778A, K778C, K778F, K778G, K778L, K778N, K778R, T779C, T779I, I780V, T781A, T781C, T781E, T781F, T781G, T781M, T781P, T781R, T781Y, A782C, A782E, A782K, A782N, A782P, A782Q, A782Y, D783A, D783C, D783E, D783R, G784A, G784F, G784L, G784S, G784T, K785C, K785I, K785S, K785V, K785W, K785Y, D786S, D786V, L787D, L787K, L787P, L787T, L787Y, S788A, S788G, S788I, Y789C, Y789D, Y789I, Y789V, I790A, I790C, I790F, I790R, I790V, E791A, E791D, E791F, E791M, E791S, E791T, E791V, E791W, V792C, V792G, V792L, V792S, V792Y, D793C, D793F, D793H, D793K, D793N, D793Y, V794C, V794D, V794L, V794Q, V794T, V794W, T795P, T795Y, D796M, D796Q, D796S, D796T, A797H, A797K, N798G, N798I, N798P, N798Q, G799D, G799K, G799L, G799M, G799Q, G799Y, H800A, H800F, H800G, H800L, H800S, H800V, I801C, I801E, I801W, V802E, V802I, V802P, V802S, V802Y, P803A, P803F, P803G, P803K, P803S, P803Y, D804E, D804G, D804K, D804N, D804S, A805C, A805F, A805G, A805I, A805N, A805P, A806F, A806I, A806Q, N807F, N807Q, N807V, N807W, R808C, R808F, R808G, R808I, R808N, R808P, R808Q, V809A, V809C, V809L, V809M, V809P, T810L, T810P, T810Q, T810R, T810Y, F811L, F811Y, D812E, D812F, D812I, D812Q, V813F, V813H, V813T, V813W, V813Y, K814G, K814H, K814I, K814L, K814P, G815A, G815F, G815M, G815P, G815V, A816C, A816D, A816F, A816I, A816N, A816V, A816W, G817C, G817H, G817I, G817N, G817S, K818D, K818F, K818L, K818Q, K818R, K818S, K818V, K818W, K818Y, L819F, L819W, V820C, V820F, V820I, V820K, V820R, V820W, G821A, G821C, G821E, G821F, G821I, G821K, G821M, G821N, G821V, G821Y, V822A, V822D, V822E, V822T, D823E, N824A, N824C, N824G, N824Q, G825A, S826A, S826F, S826G, S826I, S826L, S826R, S826W, S827C, S827Q, P828C, P828G, P828I, P828L, P828Y, D829C, D829I, D829S, D829T, D829V, H830E, H830G, H830M, H830P, H830Q, H830R, H830V, D831A, D831F, D831G, D831I, D831M, D831P, D831R, D831V, S832E, S832F, S832G, S832L, S832M, S832P, S832R, S832V, S832W, Y833C, Y833D, Y833E, Y833I, Y833K, Y833N, Y833P, Y833V, Q834F, Q834G, Q834M, A835D, A835E, A835F, A835H, A835K, A835W, D836C, D836E, D836H, D836Q, D836R, D836S, D836T, D836V, D836W, D836Y, N837D, N837F, N837G, N837H, N837L, N837P, N837T, R838D, R838F, R838G, R838K, R838M, R838N, R838S, R838W, K839A, K839C, K839D, K839E, K839G, K839L, K839N, K839P, K839R, A840G, A840I, A840P, A840V, F841C, F841D, F841I, F841K, F841W, S842A, S842L, S842M, G843A, G843C, K844A, K844G, K844L, K844W, K844Y, V845N, V845W, L846G, L846I, L846M, L846S, A847L, A847T, I848M, V849A, V849L, V849S, V849T, Q850C, Q850G, Q850I, Q850L, Q850T, Q850V, Q850Y, S851C, S851D, S851E, S851L, S851T, T852D, T852G, T852L, K853N, K853P, K853Q, K853V, E854C, E854I, E854M, E854R, A855K, A855V, A855Y, E857P, E857V, I858D, I858E, I858F, I858G, I858K, I858M, I858P, I858Q, I858Y, T859V, V860T, V860Y, T861F, T861I, T861Q, T861V, T861W, A862C, A862P, A862V, K863F, K863I, K863L, K863N, K863W, A864E, A864H, A864K, A864L, D865A, D865G, G866H, G866R, L867W, S870E, S870M, S870R, S870T, T871P, V872C, V872G, K873G, K873Y, I874G, A875R, T877A, V879P, V879S, P880S, G881W, T882A, T882M, T882R, S883L, T884A, E885V, K886E, K886L, K886V, K886W, T887A, T887D, T887F, T887G, T887N, T887R, T887V, V888A, V888D, V888G, R889G, Y892D, Y892P, Y892R, Y893E, Y893G, S894D, S894G, R895M, N896M, Y897V, Y898T, V899G, K900E, K900G, T901G, T901Q, T901R, T901V, T901Y, G902A, G902D, G902F, G902L, G902P, G902Q, G902R, G902S, G902W, N903D, N903I, K904D, K904E, K904M, K904N, K904R, K904S, K904V, K904W, P905A, P905C, P905R, P905V, P905W, P905Y, I906A, I906D, I906N, I906S, I906T, I906W, I906Y, L907F, L907S, L907Y, P908C, P908D, P908G, P908I, P908L, P908M, P908T, S909E, S909F, S909G, S909W, S909Y, D910C, D910I, D910S, D910W, V911A, V911S, E912A, E912K, E912L, E912T, E912V, V913G, V913Q, V913R, V913W, R914A, R914E, R914F, R914I, R914K, R914V, R914W, R914Y, Y915A, Y915C, Y915G, Y915I, Y915M, Y915Q, Y915V, S916G, D917C, D917E, D917F, D917L, D917M, D917Q, D917S, G918E, G918H, G918T, G918V, G918W, T919D, T919K, T919Q, T919W, T919Y, S920C, S920E, S920M, S920P, S920R, S920V, S920W, D921C, D921P, D921Q, D921V, R922A, R922G, R922M, R922V, R922W, Q923A, Q923C, Q923E, Q923L, Q923M, Q923V, Q923W, N924A, N924L, N924P, N924Q, N924S, N924W, V925A, V925C, V925E, V925G, V925K, V925N, V925S, V925W, T926G, T926P, T926R, T926S, T926V, T926W, W927C, W927G, W927P, D928A, D928E, D928H, D928L, D928Q, A929C, A929P, A929V, V930A, V930E, V930I, V930K, V930M, V930T, S931G, S931P, S931R, D932F, D932R, D932S, D932T, D932V, D933I, D933R, D933S, Q934S, Q934V, I935A, I935C, I935D, I935E, I935L, I935P, I935V, I935W, A936I, A936L, A936Q, A936R, A936Y, K937G, K937I, K937M, K937P, K937Q, K937R, K937V, A938C, A938H, A938N, A938T, A938V, A938W, G939D, G939K, S940C, S940E, S940M, S940R, S940T, S940V, S940W, F941C, F941M, F941W, S942A, S942E, S942K, S942L, S942P, S942T, S942V, V943A, V943G, V943H, V943Q, V943R, A944D, A944G, A944H, A944P, A944R, A944V, G945E, G945P, G945T, T946A, T946E, T946G, T946L, T946P, T946V, T946W, V947G, V947H, V947L, V947M, V947P, V947R, V947T, A948C, A948I, A948R, A948W, G949A, G949F, G949V, Q950D, Q950G, Q950K, Q950M, Q950W, K951D, K951G, K951P, K951Q, K951S, K951W, K951Y, I952H, I952Q, S953F, S953M, S953N, S953R, S953W, V954D, V954L, V954Q, V954S, V954T, R955A, R955C, R955E, R955K, R955Q, R955W, V956A, V956D, V956G, V956H, V956I, V956M, V956Q, V956W, T957D, T957S, T957W, M958D, M958I, M958K, I959A, I959L, I959S, I959V, I959Y, D960G, D960H, D960L, D960P, D960S, D960W, E961D, E961F, E961K, E961P, E961S, E961T, I962A, I962C, I962D, I962G, I962K, I962N, I962Q, I962T, G963C, G963E, G963L, G963P, A964C, A964E, A964H, L965C, L965E, L965G, L965K, L965M, L965P, L965Q, L965S, L965V, L965Y, L966A, L966G, L966H, L966K, L966N, L966P, L966Q, L966S, L966T, L966V, N967C, N967D, N967I, N967L, N967M, N967P, N967S, N967T, N967V, Y968G, Y968L, Y968Q, Y968V, S969C, S969D, S969G, S969H, S969I, S969L, S969M, S969P, S969Q, S969Y, A970I, A970L, S971E, S971F, S971G, S971H, S971V, S971W, P973C, P973D, P973K, P973N, P973Q, P973R, P973V, P973W, P973Y, V974C, V974E, V974G, V974N, V974T, V974Y, G975D, G975F, G975K, G975L, G975Q, G975V, T976D, T976F, T976G, T976K, T976L, T976P, T976S, T976Y, P977A, P977C, P977K, P977R, P977T, P977Y, A978F, A978G, A978M, A978N, A978P, A978R, A978S, A978Y, V979G, V979N, V979R, V979Y, L980A, L980F, L980H, L980I, L980K, L980N, L980Q, L980T, L980Y, P981L, P981M, G982A, G982H, G982M, G982P, G982Q, G982W, R984P, R984S, P985E, P985F, P985H, P985K, P985L, P985W, A986C, A986E, A986F, A986I, A986K, A986L, A986M, A986N, A986S, A986W, V987A, V987C, V987F, V987I, V987K, V987L, V987Q, V987T, L988A, L988C, L988E, L988G, L988H, L988M, L988Q, L988R, L988S, L988V, L988Y, P989A, P989C, P989D, P989G, P989H, P989I, P989M, P989N, P989Q, P989W, D990F, D990P, D990S, D990W, G991C, G991F, G991H, G991K, G991P, G991Y, T992E, T992H, T992M, T992N, T992Y, V993D, V993G, V993N, V993S, T994I, T994S, T994V, S995E, S995L, S995R, S995V, A996Q, A996R, A996V, N997A, N997C, N997E, N997K, N997L, N997S, N997V, N997W, N997Y, F998M, F998W, A999F, A999G, A999L, A999M, A999R, A999S, V1000C, V1000L, V1000M, V1000N, V1000P, V1000W, D1001G, D1001K, D1001L, D1001M, D1001Q, D1001S, D1001T, D1001V, D1001Y, W1002A, W1002D, W1002E, W1002H, W1002N, W1002P, W1002Q, W1002S, T1003F, T1003G, T1003L, T1003N, T1003P, T1003R, T1003S, T1003W, T1003Y, K1004D, K1004E, K1004F, K1004G, K1004H, K1004M, K1004P, K1004R, K1004S, K1004V, P1005I, P1005N, P1005Q, P1005V, P1005Y, A1006C, A1006I, A1006N, A1006P, A1006S, A1006V, A1006W, A1006Y, D1007C, D1007L, D1007P, D1007V, T1008G, V1009G, V1009S, Y1010A, Y1010P, Y1010R, Y1010T, N1011A, N1011S, N1011T, N1011W, T1012E, T1012H, T1012I, T1012Q, T1012Y, A1013D, A1013K, A1013Q, A1013T, A1013V, G1014E, G1014I, G1014L, G1014M, G1014V, G1014W, G1014Y, T1015A, T1015F, T1015G, T1015V, V1016C, V1016D, V1016P, K1017E, K1017G, V1018I, V1018K, V1018L, V1018M, V1018R, V1018S, V1018W, T1021C, T1021E, T1021F, T1021G, T1021K, T1021L, T1021S, T1021V, A1022H, A1022L, A1022S, A1022Y, T1023D, T1023M, T1023Q, T1023R, V1024E, V1024G, V1024H, V1024K, V1024N, V1024R, V1024S, V1024W, G1026E, G1026H, G1026L, G1026R, G1026S, G1026V, G1026Y, K1027C, K1027N, K1027Q, K1027R, K1027V, E1028G, E1028S, E1028T, F1029I, F1029K, F1029L, F1029P, F1029V, F1029W, F1029Y, K1030D, K1030F, K1030H, K1030L, K1030M, K1030W, V1031H, V1031K, V1031Y, A1033G, A1033S, A1033V, T1034G, T1034H, T1034N, T1034W, I1035D, I1035G, I1035Q, R1036G, R1036L, R1036T, R1036Y, V1037C, V1037F, V1037P, V1037Q, Q1038A, Q1038D, Q1038K, R1039S, R1039V, S1040A, S1040M, S1040N, S1040R, S1040W, Q1041P, V1042N, T1043F, T1043G, T1043N, T1043R, I1044A, I1044L, G1045S, S1046I, S1046M, S1047D, V1048C, V1048F, V1048G, V1048I, V1048M, V1048Q, G1050L, G1050S, G1050V, N1051A, N1051E, N1051K, A1052C, A1052K, A1052M, A1052P, A1052R, L1053A, L1053W, R1054C, R1054L, R1054N, L1055R, L1055T, Q1057A, Q1057E, Q1057P, Q1057R, N1058R, N1058S, N1058V, N1058W, I1059W, P1060G, P1060N, P1060Q, P1060S, P1060T, A1061E, A1061G, A1061K, A1061W, D1062A, D1062F, D1062G, D1062I, D1062L, D1062M, D1062P, D1062S, K1063D, K1063M, Q1064C, Q1064M, Q1064R, Q1064T, Q1064V, S1065A, S1065C, S1065E, S1065G, S1065T, S1065W, D1066A, D1066G, D1066M, D1066V, D1066W, T1067G, T1067M, L1068C, L1068E, L1068P, L1068Q, L1068Y, D1069G, D1069K, D1069R, D1069W, A1070P, A1070T, I1071M, I1071R, I1071W, K1072E, K1072G, K1072P, K1072Q, K1072S, D1073F, D1073L, D1073M, D1073P, D1073W, G1074I, G1074L, G1074R, S1075C, S1075G, S1075I, S1075L, S1075V, T1076C, T1076E, T1076H, T1076Q, T1076S, T1077K, T1077L, T1077R, V1078D, V1078E, V1078L, V1078W, D1079G, D1079L, N1081D, N1081E, N1081G, T1082A, T1082C, T1082E, T1082F, T1082G, T1082K, T1082N, T1082S, G1083E, G1083F, G1083L, G1083P, G1083S, G1084C, G1084M, G1084V, G1084W, G1084Y, G1085A, G1085P, G1085R, G1085S, A1086H, A1086K, A1086Q, A1086R, A1086T, N1087A, N1087E, N1087I, N1087R, N1087V, N1087W, P1088D, P1088E, P1088G, P1088R, P1088W, S1089C, S1089E, S1089G, S1089K, S1089Q, S1089R, S1089V, A1090F, A1090G, A1090I, A1090K, W1091A, W1091E, W1091G, W1091H, W1091T, W1091V, W1091Y, T1092A, T1092E, T1092G, T1092K, T1092Q, T1092S, T1092V, N1093A, N1093G, N1093L, N1093P, N1093Q, N1093T, N1093V, W1094D, W1094E, W1094P, W1094R, W1094T, A1095P, A1095R, A1095T, A1095W, Y1096A, Y1096D, Y1096H, Y1096L, Y1096R, S1097D, S1097E, S1097K, S1097L, S1097T, S1097W, K1098D, K1098F, K1098G, K1098Q, K1098S, A1099C, A1099D, A1099F, A1099S, A1099V, A1099W, G1100D, G1100E, G1100H, G1100M, G1100N, G1100T, H1101K, H1101L, H1101Q, H1101R, H1101V, N1102E, N1102F, N1102H, N1102K, N1102L, N1102Q, N1102R, N1102T, T1103A, T1103E, T1103H, T1103S, T1103W, A1104I, A1104K, A1104R, E1105L, E1105S, I1106T, I1106V, T1107C, T1107M, T1107R, T1107S, F1108D, F1108K, F1108L, F1108T, F1108W, E1109A, E1109D, E1109L, E1109W, A1111G, A1111S, E1113D, E1113G, E1113P, E1113V, Q1114E, Q1114I, Q1114L, Q1114R, Q1114S, Q1114T, Q1114V, Q1115A, Q1115K, Q1115L, Q1115P, Q1115T, Q1115W, L1116D, L1116G, L1116H, L1116K, L1116V, L1116W, G1117E, G1117I, G1117M, G1117R, G1117S, G1117T, G1117V, G1117W, Q1118A, Q1118M, Q1118S, Q1118T, Q1118W, I1119D, I1119E, I1119G, I1119N, I1119S, V1120N, V1120S, V1120T, M1121G, M1121K, M1121N, M1121P, M1121S, M1121V, M1121Y, Y1122A, Y1122C, Y1122I, Y1122K, Y1122R, Y1122V, Y1122W, F1123E, F1123H, F1123R, F1123T, F1124E, F1124R, F1124V, F1124W, R1125D, R1125E, R1125F, R1125K, R1125T, R1125V, R1125W, D1126H, D1126K, D1126L, D1126R, S1127F, S1127I, S1127K, S1127M, S1127Q, S1127T, S1127W, N1128A, N1128C, N1128R, N1128S, N1128T, N1128W, A1129E, A1129L, A1129N, A1129Q, A1129R, A1129V, V1130A, V1130G, V1130P, V1130R, V1130S, R1131A, R1131N, R1131Q, R1131S, R1131W, F1132E, F1132K, F1132M, F1132P, F1132Q, F1132T, P1133D, P1133G, P1133L, P1133Q, P1133R, P1133V, D1134E, D1134G, D1134L, A1135E, A1135K, A1135L, A1135M, A1135S, A1135W, A1135Y, G1136A, G1136E, G1136P, G1136Q, G1136T, K1137A, K1137C, K1137G, K1137L, K1137P, K1137Q, K1137R, K1137S, K1137T, K1137V, T1138R, T1138Y, K1139A, K1139L, K1139R, K1139T, I1140A, I1140C, I1140G, I1140L, I1140M, I1140P, I1140R, I1140T, Q1141A, Q1141C, Q1141G, Q1141K, Q1141N, Q1141P, Q1141T, Q1141W, I1142E, I1142R, I1142S, I1142W, I1142Y, S1143G, A1144C, A1144D, A1144E, A1144N, A1144P, A1144S, A1144T, A1144V, A1144W, D1145E, D1145R, D1145T, G1146A, G1146C, G1146D, G1146K, G1146L, G1146R, G1146V, K1147A, K1147G, K1147T, K1147V, N1148H, N1148I, N1148K, N1148P, N1148Q, N1148R, N1148S, N1148T, N1148W, W1149C, W1149G, W1149I, W1149K, W1149N, W1149Q, W1149S, W1149T, W1149V, W1149Y, T1150G, T1150K, T1150P, D1151C, D1151G, D1151R, D1151T, D1151W, L1152A, L1152C, L1152E, L1152Q, L1152W, A1153E, A1153G, A1153K, A1153L, A1154C, A1154D, A1154E, A1154G, A1154R, A1154S, T1155E, T1155L, T1155Q, T1155R, T1157V, T1157W, I1158R, I1158S, I1158W, A1159C, A1159E, A1159I, A1159P, A1159R, A1159V, A1160K, A1160L, A1160Q, A1160S, Q1161A, Q1161P, Q1161S, E1162A, E1162C, E1162D, E1162F, E1162I, E1162N, E1162Q, E1162T, E1162W, E1162Y, E1165D, E1165H, E1165L, E1165M, E1165R, E1165S, E1165W, R1166D, R1166K, R1166Q, V1167A, V1167C, V1167L, V1167P, V1167R, K1168L, K1168Q, K1168R, K1168W, P1169M, P1169R, P1169S, Y1170E, Y1170K, Y1170M, Y1170Q, Y1170R, Y1170V, T1171A, T1171G, T1171M, T1171Q, T1171R, T1171S, Y1172D, Y1172E, Y1172H, Y1172I, Y1172K, Y1172L, Y1172S, Y1172V, D1173A, D1173E, D1173F, D1173G, D1173K, D1173L, D1173P, D1173R, D1173T, D1173W, F1174D, F1174P, F1174Q, F1174R, F1174S, F1174T, F1174V, F1174W, A1175G, A1175I, A1175N, A1175Q, A1175S, A1175V, A1175Y, V1177N, V1177P, V1177S, V1177T, G1178M, G1178Q, G1178S, G1178T, A1179L, A1179P, A1179Q, A1179W, T1180A, T1180G, T1180I, T1180L, T1180M, T1180Q, T1180S, T1180Y, F1181E, F1181L, F1181V, V1182M, K1183A, K1183E, K1183T, K1183V, V1184C, V1184E, V1184K, V1184L, V1184P, V1184Q, V1184Y, T1185Q, T1185V, V1186S, N1188V, N1188W, A1189C, A1189K, A1189T, A1189V, D1190R, D1190S, D1190T, D1190Y, T1191E, T1191L, T1192H, T1192P, T1193G, P1194A, P1194E, P1194G, P1194W, S1195G, V1197A, V1198E, C1199D, C1199T, A1200G, A1200V, A1200W, L1202A, L1202C, T1203E, T1203K, E1204G, E1204S, I1205V, K1208R, K1208V, K1208W, T1209A, T1209C, T1209E, T1209K, T1209N, T1209Q, T1209R, T1209W, A1210D, A1210E, A1210G, A1210K, A1210L, A1210Q, A1210R, A1210T, A1210W, T1211C, T1211D, T1211E, T1211G, T1211H, T1211K, T1211P, T1211Q, T1211R, T1211S, T1211V, K1213A, K1213D, K1213S, K1213T, K1213W, F1214A, F1214E, F1214K, F1214L, F1214P, F1214R, F1214S, F1214V, V1215D, V1215E, V1215K, V1215L, V1215Q, V1215S, V1215W, T1216A, T1216L, T1216P, T1216Q, T1216R, N1217A, N1217D, N1217E, N1217F, N1217P, N1217R, N1217S, N1217T, T1218A, T1218C, T1218E, T1218G, T1218Q, T1218S, T1218V, T1218W, S1219A, S1219E, S1219F, S1219I, S1219K, S1219R, S1219V, A1220C, A1220G, A1220L, A1220P, A1220R, A1220V, A1221D, A1221G, A1221K, A1221L, A1221R, A1221V, A1221W, L1222A, L1222C, L1222E, L1222F, L1222Q, L1222R, L1222V, L1222W, S1223C, S1223F, S1223G, S1223K, S1223L, S1223V, S1224A, S1224D, S1224G, S1224L, S1224M, S1224P, S1224R, S1224W, L1225C, L1225D, L1225E, L1225F, L1225G, L1225K, L1225P, L1225T, L1225V, L1225W, T1226A, T1226G, T1226M, T1226P, T1226R, T1226S, T1226V, T1226Y, V1227A, V1227C, V1227D, V1227E, V1227G, V1227L, V1227P, V1227Q, V1227S, N1228A, N1228D, N1228F, N1228K, N1228L, N1228T, G1229A, G1229C, G1229E, G1229Q, G1229S, G1229V, T1230F, T1230H, T1230I, T1230K, T1230L, T1230P, T1230R, T1230S, T1230W, K1231F, K1231G, K1231L, K1231M, K1231P, K1231S, K1231W, V1232E, V1232K, V1232Q, V1232R, V1232S, V1232T, V1232W, S1233P, S1233W, D1234G, D1234K, D1234R, D1234V, S1235D, S1235E, S1235G, S1235L, S1235P, S1235R, S1235W, S1235Y, V1236A, V1236C, V1236G, V1236I, V1236P, V1236Q, V1236R, L1237D, L1237E, L1237R, L1237V, L1237W, A1238D, A1238E, A1238K, A1238L, A1238N, A1238P, A1238R, A1238S, A1238T, A1239D, A1239P, A1239R, G1240D, G1240L, G1240N, G1240Q, G1240S, G1240T, G1240W, S1241D, S1241G, S1241I, S1241L, S1241M, S1241P, Y1242C, Y1242E, Y1242K, Y1242R, Y1242S, Y1242W, N1243C, N1243L, N1243M, N1243P, N1243Q, N1243S, N1243T, N1243V, N1243W, T1244A, T1244D, T1244E, T1244G, T1244L, T1244Q, T1244S, T1244V, T1244W, A1246F, A1246M, A1246N, A1246P, A1246Q, A1246R, A1246S, A1246T, I1247A, I1247G, I1247M, I1247Q, I1247R, I1247S, I1247T, I1247V, I1247W, I1248A, I1248G, I1248K, I1248L, I1248R, I1248S, I1248Y, A1249E, A1249G, A1249H, A1249I, A1249R, A1249T, A1249V, D1250I, D1250K, D1250S, D1250T, D1250W, D1250Y, V1251I, V1251T, V1251W, K1252D, K1252G, K1252V, K1252W, A1253P, A1253V, E1254F, E1254G, E1254H, E1254L, E1254R, E1254V, G1255H, G1255M, G1255S, G1255V, E1256G, E1256M, E1256N, E1256R, E1256V, E1256W, G1257F, G1257K, G1257L, G1257Q, G1257R, G1257W, N1258C, N1258G, N1258H, N1258K, N1258S, A1259K, A1259L, A1259W, V1261I, V1261L, V1261P, V1261Q, V1261R, V1261T, T1262A, T1262F, T1262M, T1262Q, T1262R, V1263E, V1263G, V1263Q, V1263R, V1263T, V1263W, L1264A, L1264E, L1264H, L1264R, L1264S, L1264Y, P1265C, P1265K, P1265L, P1265R, P1265S, P1265V, P1265W, A1266F, A1266L, A1266P, A1266S, A1266V, H1267A, H1267E, H1267F, N1269A, N1269E, N1269K, N1269R, N1269S, N1269T, N1269W, V1270D, V1270E, V1270G, V1270I, V1270L, V1270T, V1270W, I1271A, I1271H, I1271Q, R1272E, R1272F, R1272M, R1272P, R1272V, V1273R, I1274F, I1274M, I1274R, T1275A, T1275L, T1275W, E1276R, E1276W, S1277L, S1277T, S1277W, E1278Q, E1278R, D1279G, D1279I, D1279R, D1279T, D1279V, D1279W, H1280C, H1280E, H1280G, H1280V, H1280W, V1281F, V1281I, V1281S, V1281W, T1282D, T1282L, T1282V, R1283A, R1283D, R1283E, R1283P, R1283W, K1284G, T1285A, T1285E, T1285F, T1285G, T1285M, T1285R, T1285Y, F1286A, F1286E, F1286P, F1286R, F1286S, F1286T, T1287C, T1287K, T1287L, T1287M, T1287Q, T1287R, T1287S, T1287W, I1288A, I1288D, I1288F, I1288G, I1288K, N1289A, N1289Q, N1289T, L1290A, L1290G, L1290R, L1290V, L1290W, G1291K, G1291P, G1291V, G1291W, G1291Y, T1292G, T1292L, T1292Y, E1293G, E1293K, E1293L, E1293S, E1293V, Q1294E, Q1294L, Q1294P, Q1294W, E1295K, F1296A, F1296G, F1296I, P1297F, A1298Y, D1301I, E1302G, E1302R, E1302S, R1303S, D1304A or D1304V, and
b. recovering the variant.

In preferred embodiments, the method comprises introducing into the parent lactase one or more of the substitutions listed in any one of embodiments 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144 or 146 of the “PREFERRED EMBODIMENTS” section right before the “EXAMPLES” section of the present application.

In a preferred embodiment, the parent lactase used in the method for obtaining a lactase variant is obtained from Bifidobacterium, in particular Bifidobacterium bifidum.

In a preferred embodiment, the parent lactase is the lactase of SEQ ID NO: 1.

In an embodiment, the parent lactase may have an amino acid sequence which is at least 80%, 85%, 90%, or 95%, preferably at least 97%, 98%, or 99%, more preferably at least 99.5%, 99.7%, 99.8%, 99.9%, or 100%, identical to any of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14 or SEQ ID NO: 15.

Said parent lactase may have a length of 1200-2000 amino acids, preferably 1200-1500 amino acids, more preferably 1250-1400 amino acids or 1250-1350 amino acids, even more preferably 1280-1320 amino acids, such as 1300-1320 amino acids or 1300-1310 amino acids, most preferably 1300-1305, such as 1302-1304 amino acids.

In one embodiment, the parent lactase comprises the same number of amino acid residues as the lactase of SEQ ID NO: 1.

In an embodiment, the parent lactase has a length of 850-2000 amino acids, preferably 880-1350 amino acids.

A variant obtained from the method of the invention preferably has 1-50 substitutions, more preferably 1-30, 1-20, 1-10 or 1-5, such as 1, 2, 3, 4, 5, 6, 7, 8, 9, 10, 11, 12, 13, 14, 15, 16, 17, 18, 19 or 20 substitutions, compared to the parent lactase.

A variant obtained from the method of the invention comprises an alteration, preferably a substitution, of at least one amino acid compared to the parent lactase. In some aspects, a variant obtained from the method of the invention comprises at least two substitutions compared to the parent lactase, such as at least 3 substitutions, at least 4 substitutions, at least 5 substitutions, such as at least 6, 7, 8, 9 or 10 substitutions.

A variant obtained from the method of the invention has lactase activity.

A variant obtained from the method of the invention preferably has a decreased or an increased galactose inhibition, preferably measured as a galactose inhibition improvement factor (GI-IF) of at least 1.1, such as at least 1.2, at least 1.3, at least 1.4, at least 1.5, at least 1.6, at least 1.7, at least 1.8 or at least 1.9, or at most 0.9, such as at most 0.8, at most 0.7, at most 0.6, at most 0.5, at most 0.4, at most 0.3, at most 0.2 or at most 0.1, compared to the parent lactase.

In a preferred embodiment, a variant obtained from the method of the invention has a decreased galactose inhibition, preferably measured as a galactose inhibition improvement factor (GI-IF) of at least 1.1, such as at least 1.2, at least 1.3, at least 1.4, at least 1.5, at least 1.6, at least 1.7, at least 1.8 or at least 1.9.

A variant obtained from the method of the invention preferably has a ratio of beta-galactosidase activity to transgalactosylating activity of at least 2, preferably at least 3, such as at least 4, more preferably at least 5, such as at least 10, where said ratio is measured as [Galactose]/([Glucose]−[Galactose]) after incubation of the enzyme in cow's milk at 37° C. for 3 hours.

The variants of the invention may be prepared by procedures such as those mentioned below.

Site-directed mutagenesis is a technique in which one or more (e.g., several) mutations are introduced at one or more defined sites in a polynucleotide encoding the parent.

Site-directed mutagenesis can be accomplished in vitro by PCR involving the use of oligonucleotide primers containing the desired mutation. Site-directed mutagenesis can also be performed in vitro by cassette mutagenesis involving the cleavage by a restriction enzyme at a site in the plasmid comprising a polynucleotide encoding the parent and subsequent ligation of an oligonucleotide containing the mutation in the polynucleotide. Usually the restriction enzyme that digests the plasmid and the oligonucleotide is the same, permitting sticky ends of the plasmid and the insert to ligate to one another. See, e.g., Scherer and Davis, 1979, Proc. Natl. Acad. Sci. USA 76: 4949-4955; and Barton et al., 1990, Nucleic Acids Res. 18: 7349-4966.

Site-directed mutagenesis can also be accomplished in vivo by methods known in the art. See, e.g., U.S. Patent Application Publication No. 2004/0171154; Storici et al., 2001, Nature Biotechnol. 19: 773-776; Kren et al., 1998, Nat. Med. 4: 285-290; and Calissano and Macino, 1996, Fungal Genet. Newslett. 43: 15-16.

Synthetic gene construction entails in vitro synthesis of a designed polynucleotide molecule to encode a polypeptide of interest. Gene synthesis can be performed utilizing a number of techniques, such as the multiplex microchip-based technology described by Tian et al. (2004, Nature 432: 1050-1054) and similar technologies wherein oligonucleotides are synthesized and assembled upon photo-programmable microfluidic chips.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204) and region-directed mutagenesis (Derbyshire et al., 1986, Gene 46: 145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide.

Semi-synthetic gene construction is accomplished by combining aspects of synthetic gene construction, and/or site-directed mutagenesis, and/or random mutagenesis, and/or shuffling. Semi-synthetic construction is typified by a process utilizing polynucleotide fragments that are synthesized, in combination with PCR techniques. Defined regions of genes may thus be synthesized de novo, while other regions may be amplified using site-specific mutagenic primers, while yet other regions may be subjected to error-prone PCR or non-error prone PCR amplification. Polynucleotide subsequences may then be shuffled.

The variants of the application may be prepared and identified by site saturated library methods, such described in: “Methods and applications in protein engineering”, Rodrigo M. P. Siloto, Randall J. Weselake. Biocatalysis and Agricultural Biotechnology 1 (2012) 181-189.

Stabilization of an enzyme may entail running a site saturation library (SSL) screening campaign. An SSL library allows all 20 amino acids at the given amino acid position and a library is normally constructed for each amino acid position in the protein. The number of SSL libraries are thus typically counted in the hundreds for a typical enzyme molecule. The entire campaign tends to be very time consuming and resource demanding. The process will typically require construction of libraries, plating, colony picking, sequencing, re-gridding and high-throughput screening (HTS).

The screening itself is based on the principle that an exponential decay is observed for degradation of a protein when exposed to stress conditions, e.g. elevated temperature, protease, surfactant. A stability improved variant can be found in a screening situation regardless of its initial concentration, as the stable variant after sufficient stress will always surpass the concentration of a reference sample by comparison, given a sufficiently sensitive detection method is available. The concentration may be reflected in e.g. an activity measurement.

It is clear to the skilled artisan that the term improved refers to the polypeptide variants being improved compared to a reference polypeptide, which may be the parent polypeptide or the polypeptide of SEQ ID NO 1.

### Method for Producing a Dairy Product

In one aspect, the present invention relates to a method for producing a dairy product comprising:
a) providing a milk-based substrate comprising lactose; and
b) treating said substrate with a variant of the invention.

Preferably, step b) results in a lactose reduction of at least 70%, more preferably at least 80%, even more preferably at least 90%, most preferably at least 95% or at least 98%.

In one aspect, the present invention relates to a method for producing a fermented dairy product comprising:
a) providing a milk-based substrate comprising lactose;
b) treating said substrate with a variant of the invention;
c) fermenting said substrate with a microorganism.

Step b) and step c) may be performed essentially at the same time.

Preferably, the treatment with the lactase variant results in a lactose reduction of at least 50%, preferably at least 70% or at least 90%, compared to a similar method without addition of the lactase variant.

The lactose content in the dairy product, such as the fermented dairy product, obtained in a method of the invention is preferably below 2% w/v, more preferably below 1%, even more preferably below 0.5% or below 0.1%, and most preferably below 0.05% or below 0.01% w/v.

In one aspect, the present invention relates to the use of a variant of the invention for production of a dairy product, preferably a low-lactose dairy product having a lactose content of below 2% w/v, preferably below 1%, more preferably below 0.5% or below 0.1%, and most preferably below 0.05% or below 0.01% w/v.

The dosage of the lactase variant and other conditions under which the lactase variant or a composition comprising the lactase variant is used may be determined on the basis of methods known in the art.

### Preferred Embodiments

1. A lactase variant comprising an alteration, preferably a substitution, at one or more positions corresponding to positions 1, 2, 3, 4, 5, 6, 7, 9, 10, 11, 12, 13, 14, 15, 16, 17, 19, 20, 21, 22, 23, 24, 25, 26, 27, 28, 29, 30, 31, 32, 33, 34, 35, 36, 37, 38, 39, 40, 41, 42, 43, 44, 45, 47, 48, 49, 51, 53, 54, 55, 57, 58, 59, 60, 61, 62, 63, 64, 66, 67, 68, 70, 71, 73, 74, 75, 76, 77, 78, 79, 80, 81, 83, 85, 86, 87, 88, 89, 90, 91, 92, 93, 94, 95, 96, 97, 98, 100, 101, 102, 104, 105, 106, 107, 108, 109, 110, 111, 112, 113, 114, 115, 116, 117, 118, 119, 120, 121, 122, 123, 124, 125, 126, 128, 129, 130, 131, 132, 133, 135, 136, 137, 138, 139, 140, 141, 142, 146, 148, 149, 150, 151, 153, 154, 155, 157, 158, 159, 160, 161, 162, 164, 165, 166, 167, 168, 169, 170, 171, 172, 173, 174, 175, 176, 177, 178, 179, 180, 181, 182, 183, 184, 185, 186, 187, 188, 189, 190, 191, 193, 194, 195, 196, 197, 198, 199, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 254, 255, 256, 257, 258, 259, 260, 261, 262, 263, 264, 265, 266, 267, 268, 269, 270, 271, 272, 273, 274, 275, 277, 278, 279, 280, 281, 282, 283, 284, 285, 286, 287, 288, 291, 292, 293, 294, 295, 296, 297, 298, 299, 301, 302, 303, 304, 305, 306, 307, 308, 309, 310, 311, 312, 314, 315, 316, 317, 318, 320, 321, 322, 324, 325, 326, 328, 329, 331, 335, 337, 338, 340, 341, 342, 343, 344, 345, 346, 347, 348, 349, 350, 351, 352, 353, 354, 355, 357, 359, 360, 362, 363, 364, 365, 366, 367, 368, 369, 370, 371, 372, 373, 374, 375, 376, 377, 378, 379, 380, 381, 383, 386, 387, 388, 389, 390, 391, 392, 393, 394, 395, 396, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 485, 487, 488, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 534, 535, 537, 538, 539, 540, 542, 543, 545, 546, 547, 548, 549, 550, 551, 553, 554, 555, 556, 557, 558, 559, 560, 561, 563, 564, 565, 567, 570, 571, 572, 573, 574, 575, 576, 577, 578, 579, 580, 581, 582, 583, 584, 585, 586, 587, 588, 589, 590, 591, 592, 593, 594, 595, 597, 598, 600, 604, 605, 606, 607, 608, 609, 610, 611, 612, 613, 614, 615, 616, 619, 620, 621, 624, 625, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 659, 661, 662, 667, 669, 670, 672, 673, 674, 675, 676, 677, 678, 679, 680, 681, 682, 683, 684, 685, 686, 687, 688, 689, 690, 691, 692, 693, 694, 695, 696, 697, 698, 700, 701, 703, 704, 705, 706, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 765, 766, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 857, 858, 859, 860, 861, 862, 863, 864, 865, 866, 867, 870, 871, 872, 873, 874, 875, 877, 879, 880, 881, 882, 883, 884, 885, 886, 887, 888, 889, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 973, 974, 975, 976, 977, 978, 979, 980, 981, 982, 984, 985, 986, 987, 988, 989, 990, 991, 992, 993, 994, 995, 996, 997, 998, 999, 1000, 1001, 1002, 1003, 1004, 1005, 1006, 1007, 1008, 1009, 1010, 1011, 1012, 1013, 1014, 1015, 1016, 1017, 1018, 1021, 1022, 1023, 1024, 1026, 1027, 1028, 1029, 1030, 1031, 1033, 1034, 1035, 1036, 1037, 1038, 1039, 1040, 1041, 1042, 1043, 1044, 1045, 1046, 1047, 1048, 1050, 1051, 1052, 1053, 1054, 1055, 1057, 1058, 1059, 1060, 1061, 1062, 1063, 1064, 1065, 1066, 1067, 1068, 1069, 1070, 1071, 1072, 1073, 1074, 1075, 1076, 1077, 1078, 1079, 1081, 1082, 1083, 1084, 1085, 1086, 1087, 1088, 1089, 1090, 1091, 1092, 1093, 1094, 1095, 1096, 1097, 1098, 1099, 1100, 1101, 1102, 1103, 1104, 1105, 1106, 1107, 1108, 1109, 1111, 1113, 1114, 1115, 1116, 1117, 1118, 1119, 1120, 1121, 1122, 1123, 1124, 1125, 1126, 1127, 1128, 1129, 1130, 1131, 1132, 1133, 1134, 1135, 1136, 1137, 1138, 1139, 1140, 1141, 1142, 1143, 1144, 1145, 1146, 1147, 1148, 1149, 1150, 1151, 1152, 1153, 1154, 1155, 1157, 1158, 1159, 1160, 1161, 1162, 1165, 1166, 1167, 1168, 1169, 1170, 1171, 1172, 1173, 1174, 1175, 1177, 1178, 1179, 1180, 1181, 1182, 1183, 1184, 1185, 1186, 1188, 1189, 1190, 1191, 1192, 1193, 1194, 1195, 1197, 1198, 1199, 1200, 1202, 1203, 1204, 1205, 1208, 1209, 1210, 1211, 1213, 1214, 1215, 1216, 1217, 1218, 1219, 1220, 1221, 1222, 1223, 1224, 1225, 1226, 1227, 1228, 1229, 1230, 1231, 1232, 1233, 1234, 1235, 1236, 1237, 1238, 1239, 1240, 1241, 1242, 1243, 1244, 1246, 1247, 1248, 1249, 1250, 1251, 1252, 1253, 1254, 1255, 1256, 1257, 1258, 1259, 1261, 1262, 1263, 1264, 1265, 1266, 1267, 1269, 1270, 1271, 1272, 1273, 1274, 1275, 1276, 1277, 1278, 1279, 1280, 1281, 1282, 1283, 1284, 1285, 1286, 1287, 1288, 1289, 1290, 1291, 1292, 1293, 1294, 1295, 1296, 1297, 1298, 1301, 1302, 1303 or 1304 of the polypeptide of SEQ ID NO: 1.

2. The lactase variant of the preceding embodiment, wherein the variant has a decreased or an increased galactose inhibition, preferably measured as a galactose inhibition improvement factor (GI-IF) of at least 1.1 or at most 0.9, compared to the lactase of SEQ ID NO: 1.

3. The lactase variant of embodiment 1 comprising an alteration, preferably a substitution, at one or more positions corresponding to positions 2, 3, 4, 5, 6, 7, 9, 10, 11, 12, 13, 14, 15, 16, 17, 21, 22, 23, 24, 25, 26, 27, 28, 29, 31, 32, 33, 34, 35, 36, 37, 38, 39, 40, 41, 42, 43, 44, 45, 47, 48, 49, 51, 54, 55, 57, 58, 59, 60, 61, 62, 63, 64, 66, 67, 68, 70, 71, 73, 74, 75, 76, 77, 78, 79, 80, 81, 83, 85, 86, 87, 88, 89, 90, 91, 92, 93, 94, 95, 96, 97, 98, 100, 101, 102, 104, 105, 106, 107, 108, 109, 110, 111, 112, 113, 114, 115, 116, 117, 118, 119, 120, 121, 122, 123, 124, 125, 126, 128, 129, 130, 131, 132, 133, 135, 136, 137, 138, 139, 140, 141, 142, 146, 148, 150, 151, 153, 154, 155, 157, 158, 159, 160, 161, 162, 164, 165, 166, 167, 168, 169, 170, 171, 172, 173, 174, 175, 176, 177, 178, 179, 180, 181, 182, 183, 184, 185, 186, 187, 188, 189, 190, 191, 193, 194, 195, 196, 197, 198, 199, 201, 202, 203, 204, 206, 207, 208, 209, 210, 211, 212, 213, 214, 215, 216, 217, 218, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 254, 255, 256, 257, 258, 259, 260, 261, 262, 263, 265, 267, 268, 269, 271, 272, 273, 274, 275, 277, 278, 279, 280, 281, 282, 283, 284, 285, 286, 287, 288, 291, 292, 293, 294, 295, 296, 297, 298, 299, 301, 302, 303, 304, 305, 306, 307, 308, 310, 311, 312, 314, 315, 316, 317, 318, 320, 321, 322, 324, 325, 326, 328, 329, 331, 335, 337, 338, 340, 341, 342, 343, 344, 345, 346, 347, 348, 349, 350, 351, 352, 353, 354, 355, 357, 359, 360, 362, 363, 364, 365, 366, 367, 368, 369, 370, 371, 372, 373, 374, 375, 376, 377, 378, 379, 380, 381, 383, 386, 387, 388, 389, 390, 391, 392, 393, 394, 395, 396, 398, 399, 400, 401, 402, 403, 404, 406, 407, 408, 409, 410, 411, 412, 413, 414, 415, 416, 417, 418, 419, 421, 422, 423, 424, 425, 426, 427, 428, 429, 430, 431, 432, 433, 434, 435, 436, 437, 438, 439, 440, 441, 442, 443, 444, 445, 446, 448, 449, 450, 451, 452, 453, 454, 455, 456, 457, 459, 461, 462, 463, 464, 465, 466, 467, 469, 470, 471, 472, 473, 474, 475, 476, 477, 478, 479, 480, 485, 487, 488, 491, 492, 493, 494, 495, 496, 497, 498, 499, 500, 501, 502, 503, 504, 505, 506, 507, 508, 509, 510, 511, 512, 514, 516, 517, 518, 519, 520, 521, 524, 525, 526, 527, 528, 529, 530, 531, 534, 535, 537, 538, 539, 540, 542, 543, 545, 546, 547, 548, 549, 550, 551, 553, 554, 555, 556, 557, 558, 559, 560, 561, 563, 564, 565, 567, 570, 571, 572, 573, 574, 575, 576, 577, 578, 579, 580, 581, 582, 583, 584, 585, 586, 587, 588, 589, 590, 591, 592, 593, 594, 595, 597, 598, 600, 604, 605, 606, 608, 609, 610, 611, 612, 613, 631, 633, 634, 635, 636, 637, 638, 639, 640, 641, 642, 643, 644, 645, 646, 647, 648, 649, 650, 651, 652, 653, 654, 655, 656, 657, 659, 661, 662, 669, 670, 672, 673, 674, 675, 676, 677, 678, 679, 680, 681, 682, 683, 684, 685, 686, 687, 688, 689, 690, 693, 694, 695, 696, 697, 698, 700, 701, 703, 704, 705, 706, 708, 709, 710, 711, 712, 713, 714, 715, 718, 720, 721, 723, 724, 725, 726, 727, 728, 729, 730, 731, 732, 733, 734, 735, 736, 737, 738, 739, 740, 741, 742, 743, 744, 745, 746, 747, 748, 750, 751, 752, 753, 755, 756, 757, 758, 759, 760, 761, 762, 765, 766, 768, 769, 770, 771, 772, 773, 774, 775, 776, 777, 778, 779, 780, 781, 782, 783, 784, 785, 786, 787, 788, 789, 790, 791, 792, 793, 794, 795, 796, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 840, 841, 842, 843, 844, 845, 846, 847, 848, 849, 850, 851, 852, 853, 854, 855, 857, 858, 859, 860, 861, 862, 863, 864, 865, 866, 870, 871, 872, 873, 874, 875, 877, 879, 880, 881, 882, 883, 884, 885, 886, 887, 888, 889, 893, 894, 895, 896, 897, 898, 899, 900, 901, 902, 903, 904, 905, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 973, 974, 975, 976, 977, 978, 979, 980, 981, 984, 985, 986, 987, 988, 989, 990, 991, 992, 993, 994, 995, 996, 997, 998, 999, 1000, 1002, 1003, 1004, 1005, 1006, 1007, 1008, 1009, 1010, 1011, 1012, 1013, 1014, 1015, 1016, 1017, 1018, 1021, 1022, 1023, 1024, 1026, 1027, 1028, 1029, 1030, 1031, 1033, 1034, 1035, 1036, 1037, 1038, 1039, 1040, 1041, 1042, 1043, 1044, 1045, 1046, 1047, 1048, 1050, 1051, 1052, 1053, 1055, 1057, 1058, 1059, 1060, 1062, 1063, 1064, 1065, 1066, 1067, 1068, 1070, 1071, 1072, 1073, 1074, 1075, 1076, 1077, 1078, 1079, 1081, 1082, 1083, 1084, 1085, 1086, 1087, 1088, 1089, 1090, 1091, 1092, 1093, 1094, 1095, 1096, 1097, 1098, 1099, 1100, 1101, 1102, 1103, 1104, 1105, 1106, 1107, 1108, 1109, 1111, 1113, 1114, 1115, 1116, 1117, 1118, 1119, 1120, 1121, 1122, 1123, 1124, 1125, 1126, 1127, 1128, 1129, 1130, 1131, 1132, 1133, 1134, 1135, 1136, 1137, 1138, 1139, 1140, 1141, 1142, 1143, 1144, 1145, 1146, 1147, 1148, 1149, 1150, 1151, 1152, 1153, 1154, 1155, 1157, 1158, 1159, 1161, 1162, 1166, 1167, 1168, 1169, 1170, 1171, 1172, 1173, 1174, 1175, 1177, 1178, 1179, 1180, 1181, 1182, 1184, 1185, 1186, 1188, 1189, 1190, 1191, 1192, 1193, 1194, 1195, 1197, 1198, 1199, 1202, 1203, 1204, 1205, 1208, 1209, 1210, 1211, 1213, 1214, 1215, 1217, 1218, 1219, 1220, 1221, 1222, 1223, 1224, 1225, 1226, 1227, 1228, 1230, 1231, 1232, 1233, 1234, 1235, 1236, 1237, 1238, 1239, 1240, 1241, 1242, 1243, 1244, 1246, 1247, 1248, 1249, 1250, 1251, 1252, 1253, 1254, 1255, 1256, 1257, 1258, 1259, 1261, 1262, 1263, 1264, 1265, 1266, 1267, 1269, 1270, 1271, 1272, 1273, 1274, 1275, 1276, 1277, 1278, 1279, 1280, 1281, 1282, 1283, 1284, 1285, 1286, 1287, 1288, 1289, 1290, 1291, 1292, 1293, 1294, 1295, 1296, 1297, 1298, 1301, 1302, 1303 or 1304 of the polypeptide of SEQ ID NO: 1.

4. The lactase variant of the preceding embodiment, wherein the variant has a decreased or an increased galactose inhibition, preferably measured as a galactose inhibition improvement factor (GI-IF) of at least 1.1 or at most 0.9, compared to the lactase of SEQ ID NO: 1.

5. The lactase variant of embodiment 1 comprising an alteration, preferably a substitution, at one or more positions corresponding to positions 1, 2, 3, 4, 5, 6, 7, 9, 10, 12, 13, 14, 15, 16, 17, 19, 20, 21, 22, 23, 24, 25, 26, 27, 28, 29, 30, 31, 32, 33, 34, 35, 36, 37, 38, 39, 40, 41, 42, 43, 44, 45, 47, 49, 51, 53, 55, 57, 58, 59, 60, 61, 62, 63, 64, 66, 70, 71, 73, 74, 75, 76, 77, 78, 79, 83, 85, 86, 87, 88, 89, 90, 91, 92, 93, 94, 95, 96, 97, 98, 100, 101, 102, 104, 105, 106, 107, 108, 109, 110, 111, 112, 114, 115, 116, 117, 118, 119, 120, 121, 124, 125, 126, 129, 130, 131, 132, 137, 138, 140, 141, 142, 148, 149, 150, 151, 154, 157, 158, 159, 160, 161, 162, 164, 165, 166, 167, 169, 170, 171, 172, 173, 174, 175, 176, 177, 178, 179, 180, 181, 183, 184, 185, 186, 187, 188, 189, 190, 191, 193, 194, 195, 196, 197, 198, 199, 201, 202, 203, 204, 205, 206, 207, 213, 215, 216, 217, 218, 219, 220, 221, 222, 223, 224, 225, 226, 228, 231, 232, 234, 235, 236, 237, 238, 240, 242, 243, 247, 248, 251, 252, 257, 260, 261, 262, 263, 264, 266, 267, 268, 270, 271, 272, 273, 274, 275, 277, 280, 281, 282, 283, 285, 286, 291, 292, 293, 294, 295, 296, 297, 298, 299, 301, 302, 303, 304, 305, 307, 308, 309, 310, 311, 312, 314, 315, 316, 317, 318, 320, 321, 322, 324, 325, 326, 328, 331, 335, 337, 338, 340, 341, 342, 343, 344, 345, 346, 347, 348, 349, 350, 351, 352, 353, 354, 355, 357, 359, 360, 362, 363, 364, 365, 366, 367, 368, 369, 370, 371, 372, 373, 374, 375, 376, 377, 378, 379, 380, 381, 383, 386, 387, 388, 389, 390, 393, 395, 396, 398, 399, 400, 401, 402, 403, 404, 405, 406, 407, 410, 411, 412, 414, 415, 416, 417, 422, 423, 426, 427, 429, 430, 431, 432, 433, 434, 435, 436, 437, 438, 439, 440, 441, 442, 443, 444, 446, 448, 449, 450, 451, 452, 453, 454, 455, 456, 457, 458, 459, 460, 461, 462, 464, 465, 466, 467, 468, 469, 471, 472, 473, 474, 475, 476, 477, 478, 479, 480, 485, 487, 488, 493, 494, 495, 497, 499, 505, 506, 507, 508, 509, 510, 511, 512, 513, 515, 516, 517, 518, 519, 520, 521, 523, 524, 525, 526, 527, 528, 529, 535, 537, 538, 540, 545, 547, 549, 550, 553, 555, 556, 558, 559, 560, 564, 565, 567, 570, 572, 574, 575, 577, 578, 580, 581, 582, 583, 584, 586, 588, 589, 591, 592, 593, 594, 597, 598, 600, 604, 605, 607, 608, 609, 610, 612, 613, 614, 615, 619, 621, 625, 627, 628, 629, 630, 631, 632, 633, 634, 636, 637, 639, 643, 644, 645, 646, 647, 648, 649, 650, 651, 652, 653, 654, 655, 656, 657, 661, 662, 667, 669, 672, 673, 674, 675, 676, 677, 679, 680, 682, 683, 684, 686, 687, 688, 689, 690, 691, 692, 693, 694, 695, 696, 697, 698, 700, 701, 703, 705, 706, 708, 710, 711, 712, 713, 714, 715, 716, 717, 718, 719, 720, 721, 722, 723, 724, 725, 726, 727, 728, 729, 731, 732, 733, 734, 735, 737, 738, 739, 740, 741, 742, 743, 744, 745, 746, 747, 749, 750, 751, 752, 754, 755, 756, 757, 758, 759, 760, 761, 762, 765, 766, 769, 770, 771, 772, 773, 774, 775, 776, 777, 778, 779, 781, 782, 783, 785, 786, 787, 789, 790, 791, 792, 793, 794, 796, 797, 798, 799, 800, 801, 802, 803, 804, 805, 806, 807, 808, 809, 810, 811, 813, 814, 815, 816, 817, 818, 819, 820, 821, 822, 823, 824, 825, 826, 828, 829, 830, 831, 832, 833, 834, 835, 836, 838, 839, 840, 841, 842, 844, 847, 848, 850, 851, 852, 853, 854, 855, 858, 860, 861, 862, 864, 865, 866, 867, 870, 872, 873, 874, 875, 879, 882, 883, 885, 886, 887, 888, 889, 892, 893, 894, 897, 898, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 933, 934, 935, 936, 937, 938, 939, 940, 941, 942, 943, 946, 947, 948, 949, 950, 951, 952, 953, 954, 955, 956, 957, 959, 960, 961, 962, 963, 965, 966, 967, 968, 969, 970, 971, 973, 974, 975, 976, 977, 978, 979, 980, 981, 984, 985, 986, 987, 988, 989, 990, 991, 992, 993, 995, 996, 997, 998, 1000, 1001, 1002, 1003, 1004, 1005, 1006, 1007, 1008, 1010, 1011, 1012, 1013, 1014, 1015, 1016, 1017, 1018, 1021, 1022, 1024, 1026, 1027, 1028, 1029, 1030, 1031, 1033, 1034, 1035, 1036, 1037, 1038, 1039, 1040, 1041, 1042, 1043, 1044, 1046, 1048, 1051, 1052, 1053, 1054, 1055, 1057, 1058, 1060, 1061, 1062, 1063, 1064, 1065, 1066, 1067, 1068, 1069, 1070, 1071, 1072, 1073, 1074, 1075, 1076, 1077, 1078, 1079, 1081, 1082, 1083, 1084, 1085, 1086, 1087, 1088, 1089, 1090, 1091, 1093, 1094, 1095, 1096, 1097, 1098, 1099, 1100, 1101, 1102, 1103, 1105, 1109, 1111, 1113, 1114, 1115, 1116, 1117, 1118, 1119, 1120, 1121, 1123, 1124, 1125, 1127, 1128, 1129, 1130, 1131, 1132, 1133, 1135, 1136, 1137, 1138, 1140, 1141, 1142, 1143, 1144, 1145, 1146, 1148, 1149, 1150, 1151, 1152, 1153, 1154, 1158, 1159, 1161, 1162, 1165, 1166, 1167, 1168, 1169, 1170, 1172, 1173, 1174, 1175, 1178, 1179, 1180, 1181, 1182, 1183, 1184, 1185, 1186, 1188, 1189, 1190, 1191, 1193, 1194, 1197, 1199, 1200, 1203, 1204, 1205, 1208, 1209, 1210, 1211, 1213, 1214, 1215, 1216, 1217, 1218, 1219, 1220, 1221, 1222, 1224, 1225, 1226, 1227, 1229, 1230, 1231, 1232, 1234, 1235, 1236, 1237, 1238, 1239, 1240, 1243, 1247, 1255, 1259, 1261, 1262, 1263, 1264, 1265, 1273, 1274, 1275, 1276, 1277, 1278, 1279, 1280, 1281, 1284, 1285, 1286, 1287, 1288, 1293, 1294, 1295, 1296, 1302, 1303 or 1304 of the polypeptide of SEQ ID NO: 1.

6. The lactase variant of the preceding embodiment, wherein the variant has a decreased galactose inhibition, preferably measured as a galactose inhibition improvement factor (GI-IF) of at least 1.1, compared to the lactase of SEQ ID NO: 1.

7. The lactase variant of embodiment 1 comprising an alteration, preferably a substitution, at one or more positions corresponding to positions 2, 3, 4, 5, 6, 7, 9, 10, 12, 13, 14, 15, 16, 17, 21, 22, 23, 24, 25, 26, 27, 28, 29, 31, 32, 33, 34, 35, 36, 37, 38, 39, 40, 41, 42, 43, 44, 45, 47, 49, 51, 55, 57, 58, 59, 60, 61, 62, 63, 64, 66, 70, 71, 73, 74, 75, 76, 77, 78, 79, 83, 85, 86, 87, 88, 89, 90, 91, 92, 93, 94, 95, 96, 97, 98, 100, 101, 102, 104, 105, 106, 107, 108, 109, 110, 111, 112, 114, 115, 116, 117, 118, 119, 120, 121, 124, 125, 126, 129, 130, 131, 132, 137, 138, 140, 141, 142, 148, 150, 151, 154, 157, 158, 159, 160, 161, 162, 164, 165, 166, 167, 169, 170, 171, 172, 173, 174, 175, 176, 177, 178, 179, 180, 181, 183, 184, 185, 186, 187, 188, 189, 190, 191, 193, 194, 195, 196, 197, 198, 199, 201, 202, 203, 204, 206, 207, 213, 215, 216, 217, 218, 220, 221, 222, 223, 224, 225, 226, 228, 231, 232, 234, 235, 236, 237, 238, 240, 242, 243, 247, 248, 251, 252, 257, 260, 261, 262, 263, 267, 268, 271, 272, 273, 274, 275, 277, 280, 281, 282, 283, 285, 286, 291, 292, 293, 294, 295, 296, 297, 298, 299, 301, 302, 303, 304, 305, 307, 308, 310, 311, 312, 314, 315, 316, 317, 318, 320, 321, 322, 324, 325, 326, 328, 331, 335, 337, 338, 340, 341, 342, 343, 344, 345, 346, 347, 348, 349, 350, 351, 352, 353, 354, 355, 357, 359, 360, 362, 363, 364, 365, 366, 367, 368, 369, 370, 371, 372, 373, 374, 375, 376, 377, 378, 379, 380, 381, 383, 386, 387, 388, 389, 390, 393, 395, 396, 398, 399, 400, 401, 402, 403, 404, 406, 407, 410, 411, 412, 414, 415, 416, 417, 422, 423, 426, 427, 429, 430, 431, 432, 433, 434, 435, 436, 437, 438, 439, 440, 441, 442, 443, 444, 446, 448, 449, 450, 451, 452, 453, 454, 455, 456, 457, 459, 461, 462, 464, 465, 466, 467, 469, 471, 472, 473, 474, 475, 476, 477, 478, 479, 480, 485, 487, 488, 493, 494, 495, 497, 499, 505, 506, 507, 508, 509, 510, 511, 512, 516, 517, 518, 519, 520, 521, 524, 525, 526, 527, 528, 529, 535, 537, 538, 540, 545, 547, 549, 550, 553, 555, 556, 558, 559, 560, 564, 565, 567, 570, 572, 574, 575, 577, 578, 580, 581, 582, 583, 584, 586, 588, 589, 591, 592, 593, 594, 597, 598, 600, 604, 605, 608, 609, 610, 612, 613, 631, 633, 634, 636, 637, 639, 643, 644, 645, 646, 647, 648, 649, 650, 651, 652, 653, 654, 655, 656, 657, 661, 662, 669, 672, 673, 674, 675, 676, 677, 679, 680, 682, 683, 684, 686, 687, 688, 689, 690, 693, 694, 695, 696, 697, 698, 700, 701, 703, 705, 706, 708, 710, 711, 712, 713, 714, 715, 718, 720, 721, 723, 724, 725, 726, 727, 728, 729, 731, 732, 733, 734, 735, 737, 738, 739, 740, 741, 742, 743, 744, 745, 746, 747, 750, 751, 752, 755, 756, 757, 758, 759, 760, 761, 762, 765, 766, 769, 770, 771, 772, 773, 774, 775, 776, 777, 778, 779, 781, 782, 783, 785, 786, 787, 789, 790, 791, 792, 793, 794, 796, 798, 799, 800, 801, 802, 803, 804, 805, 806, 807, 808, 809, 810, 811, 813, 814, 815, 816, 817, 818, 819, 820, 821, 822, 823, 824, 825, 826, 828, 829, 830, 831, 832, 833, 834, 835, 836, 838, 840, 841, 842, 844, 847, 848, 850, 851, 852, 853, 854, 855, 858, 860, 861, 862, 864, 865, 866, 870, 872, 873, 874, 875, 879, 882, 883, 885, 886, 887, 888, 889, 893, 894, 897, 898, 900, 901, 902, 903, 904, 905, 907, 908, 909, 910, 911, 912, 913, 914, 915, 916, 917, 918, 919, 920, 921, 922, 923, 924, 925, 926, 927, 928, 929, 930, 931, 933, 934, 935, 936, 937, 938, 939, 940, 941, 942, 943, 946, 947, 948, 949, 950, 951, 952, 953, 954, 955, 956, 957, 959, 960, 961, 962, 963, 965, 966, 967, 968, 969, 970, 973, 974, 975, 976, 977, 978, 979, 980, 981, 984, 985, 986, 987, 988, 989, 990, 991, 992, 993, 995, 996, 997, 998, 1000, 1002, 1003, 1004, 1005, 1006, 1007, 1008, 1010, 1011, 1012, 1013, 1014, 1015, 1016, 1017, 1018, 1021, 1022, 1024, 1026, 1027, 1028, 1029, 1030, 1031, 1033, 1034, 1035, 1036, 1037, 1038, 1039, 1040, 1041, 1042, 1043, 1044, 1046, 1048, 1051, 1052, 1053, 1055, 1057, 1058, 1060, 1062, 1063, 1064, 1065, 1066, 1067, 1068, 1070, 1071, 1072, 1073, 1074, 1075, 1076, 1077, 1078, 1079, 1081, 1082, 1083, 1084, 1085, 1086, 1087, 1088, 1089, 1090, 1091, 1093, 1094, 1095, 1096, 1097, 1098, 1099, 1100, 1101, 1102, 1103, 1105, 1109, 1111, 1113, 1114, 1115, 1116, 1117, 1118, 1119, 1120, 1121, 1123, 1124, 1125, 1127, 1128, 1129, 1130, 1131, 1132, 1133, 1135, 1136, 1137, 1138, 1140, 1141, 1142, 1143, 1144, 1145, 1146, 1148, 1149, 1150, 1151, 1152, 1153, 1154, 1158, 1159, 1161, 1162, 1166, 1167, 1168, 1169, 1170, 1172, 1173, 1174, 1175, 1178, 1179, 1180, 1181, 1182, 1184, 1185, 1186, 1188, 1189, 1190, 1191, 1193, 1194, 1197, 1199, 1203, 1204, 1205, 1208, 1209, 1210, 1211, 1213, 1214, 1215, 1217, 1218, 1219, 1220, 1221, 1222, 1224, 1225, 1226, 1227, 1230, 1231, 1232, 1234, 1235, 1236, 1237, 1238, 1239, 1240, 1243, 1247, 1255, 1259, 1261, 1262, 1263, 1264, 1265, 1273, 1274, 1275, 1276, 1277, 1278, 1279, 1280, 1281, 1284, 1285, 1286, 1287, 1288, 1293, 1294, 1295, 1296, 1302, 1303 or 1304 of the polypeptide of SEQ ID NO: 1.

8. The lactase variant of the preceding embodiment, wherein the variant has a decreased galactose inhibition, preferably measured as a galactose inhibition improvement factor (GI-IF) of at least 1.1, compared to the lactase of SEQ ID NO: 1.

9. The lactase variant of embodiment 1 comprising an alteration, preferably a substitution, at one or more positions corresponding to positions 1, 2, 3, 5, 6, 7, 9, 10, 11, 12, 13, 14, 15, 16, 17, 19, 20, 21, 22, 23, 24, 25, 26, 27, 28, 29, 30, 31, 32, 33, 34, 35, 36, 38, 39, 40, 41, 42, 43, 44, 45, 47, 49, 51, 53, 55, 57, 58, 59, 60, 61, 62, 63, 64, 66, 68, 70, 71, 73, 74, 75, 76, 77, 78, 79, 80, 81, 83, 85, 86, 87, 88, 89, 90, 91, 92, 93, 94, 95, 96, 97, 98, 100, 101, 102, 106, 107, 108, 109, 110, 111, 112, 113, 114, 115, 116, 117, 118, 119, 120, 121, 122, 123, 124, 125, 128, 129, 130, 132, 136, 137, 138, 139, 140, 141, 142, 146, 148, 149, 150, 153, 154, 155, 157, 158, 159, 161, 162, 164, 165, 166, 167, 169, 170, 171, 172, 173, 174, 176, 177, 178, 179, 180, 181, 182, 183, 184, 185, 186, 187, 188, 189, 190, 193, 194, 195, 196, 197, 198, 199, 201, 202, 203, 204, 205, 206, 207, 208, 209, 210, 211, 212, 213, 214, 215, 217, 218, 219, 220, 221, 222, 223, 226, 228, 229, 230, 231, 232, 233, 234, 235, 236, 237, 238, 239, 240, 241, 242, 243, 244, 245, 246, 247, 248, 249, 250, 251, 252, 254, 255, 256, 257, 258, 259, 260, 261, 262, 264, 265, 266, 267, 268, 269, 270, 272, 273, 275, 277, 278, 279, 280, 281, 282, 283, 284, 285, 286, 288, 291, 292, 293, 294, 295, 296, 297, 298, 301, 302, 303, 304, 305, 306, 307, 308, 309, 310, 311, 312, 314, 315, 316, 317, 318, 320, 321, 322, 324, 325, 326, 328, 329, 331, 335, 337, 338, 340, 341, 342, 343, 344, 345, 346, 347, 348, 349, 350, 351, 352, 353, 354, 355, 357, 359, 360, 362, 363, 364, 365, 366, 367, 368, 369, 370, 371, 372, 373, 374, 375, 376, 377, 379, 380, 381, 383, 386, 387, 388, 389, 390, 391, 392, 393, 394, 395, 396, 398, 399, 400, 401, 402, 403, 404, 405, 406, 407, 408, 409, 410, 411, 413, 414, 415, 416, 417, 418, 419, 420, 421, 422, 423, 424, 426, 427, 428, 429, 430, 431, 432, 433, 434, 435, 436, 437, 439, 440, 441, 442, 443, 444, 445, 446, 448, 449, 450, 451, 452, 453, 454, 455, 456, 457, 458, 459, 460, 461, 462, 463, 464, 465, 466, 467, 468, 469, 470, 471, 472, 474, 475, 477, 478, 479, 480, 485, 487, 488, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 524, 525, 526, 527, 528, 529, 530, 531, 534, 535, 537, 538, 539, 540, 542, 543, 545, 546, 547, 548, 549, 550, 551, 553, 554, 555, 556, 557, 558, 559, 561, 563, 564, 565, 567, 570, 572, 573, 574, 575, 576, 577, 578, 579, 580, 581, 582, 583, 584, 585, 586, 587, 588, 589, 590, 591, 592, 593, 594, 595, 597, 598, 600, 604, 606, 607, 608, 609, 610, 611, 612, 614, 615, 616, 619, 620, 624, 625, 627, 628, 629, 630, 631, 632, 634, 635, 637, 638, 639, 644, 645, 646, 647, 648, 650, 651, 652, 653, 654, 655, 656, 657, 659, 661, 662, 667, 669, 670, 673, 674, 675, 676, 677, 678, 679, 680, 681, 682, 683, 684, 686, 687, 688, 689, 690, 691, 692, 694, 695, 696, 697, 698, 700, 701, 704, 705, 706, 708, 709, 710, 711, 712, 713, 714, 715, 716, 717, 718, 719, 720, 721, 722, 723, 726, 727, 728, 729, 731, 732, 733, 734, 735, 737, 738, 739, 740, 741, 742, 744, 745, 747, 750, 751, 752, 754, 756, 757, 759, 760, 766, 769, 770, 771, 772, 773, 774, 775, 776, 777, 778, 780, 781, 782, 783, 784, 785, 786, 787, 788, 789, 790, 791, 792, 793, 794, 795, 796, 798, 799, 800, 801, 802, 803, 804, 805, 806, 807, 808, 809, 810, 811, 812, 813, 814, 815, 816, 817, 818, 819, 820, 821, 822, 825, 826, 827, 828, 829, 830, 831, 832, 833, 834, 835, 836, 837, 838, 839, 840, 841, 842, 843, 844, 845, 846, 849, 850, 853, 854, 857, 858, 859, 860, 861, 862, 863, 864, 865, 866, 867, 870, 871, 872, 873, 874, 877, 880, 881, 882, 883, 884, 885, 886, 887, 888, 889, 892, 893, 894, 896, 897, 899, 900, 901, 902, 903, 904, 905, 906, 907, 908, 910, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 965, 966, 967, 968, 969, 970, 971, 973, 974, 975, 976, 977, 978, 979, 980, 981, 982, 985, 986, 987, 988, 989, 990, 991, 992, 994, 995, 996, 997, 998, 999, 1000, 1001, 1002, 1003, 1004, 1005, 1006, 1007, 1009, 1010, 1011, 1012, 1013, 1014, 1015, 1016, 1018, 1021, 1022, 1023, 1024, 1026, 1027, 1029, 1030, 1031, 1034, 1035, 1036, 1037, 1041, 1042, 1043, 1044, 1046, 1048, 1050, 1051, 1052, 1053, 1055, 1057, 1058, 1059, 1060, 1061, 1062, 1063, 1064, 1065, 1066, 1067, 1068, 1070, 1071, 1072, 1073, 1074, 1075, 1076, 1078, 1082, 1083, 1084, 1085, 1086, 1087, 1088, 1089, 1090, 1091, 1092, 1093, 1094, 1095, 1096, 1097, 1098, 1099, 1100, 1101, 1102, 1103, 1104, 1105, 1106, 1107, 1108, 1109, 1111, 1113, 1114, 1115, 1116, 1117, 1118, 1119, 1120, 1121, 1122, 1123, 1124, 1125, 1126, 1127, 1128, 1129, 1130, 1131, 1132, 1133, 1134, 1135, 1136, 1137, 1138, 1140, 1141, 1142, 1143, 1144, 1145, 1146, 1147, 1148, 1149, 1150, 1151, 1152, 1153, 1154, 1155, 1158, 1159, 1160, 1161, 1162, 1165, 1167, 1168, 1169, 1170, 1171, 1172, 1173, 1174, 1175, 1177, 1178, 1179, 1180, 1182, 1183, 1184, 1186, 1188, 1189, 1190, 1191, 1192, 1197, 1198, 1199, 1200, 1202, 1203, 1204, 1205, 1208, 1209, 1210, 1211, 1213, 1214, 1215, 1216, 1217, 1218, 1219, 1220, 1221, 1222, 1223, 1224, 1225, 1226, 1227, 1228, 1229, 1230, 1231, 1232, 1233, 1234, 1235, 1236, 1237, 1238, 1239, 1240, 1241, 1242, 1243, 1244, 1246, 1247, 1248, 1249, 1250, 1251, 1252, 1254, 1255, 1256, 1257, 1258, 1259, 1261, 1262, 1263, 1264, 1265, 1266, 1267, 1269, 1270, 1271, 1272, 1273, 1274, 1275, 1277, 1278, 1279, 1280, 1281, 1282, 1283, 1284, 1285, 1286, 1287, 1288, 1289, 1290, 1291, 1292, 1293, 1294, 1295, 1296, 1297, 1302, 1303 or 1304 of the polypeptide of SEQ ID NO: 1.

10. The lactase variant of the preceding embodiment, wherein the variant has a decreased or an increased galactose inhibition, preferably measured as a galactose inhibition improvement factor (GI-IF) of at least 1.2 or at most 0.8, compared to the lactase of SEQ ID NO: 1.

11. The lactase variant of embodiment 1 comprising an alteration, preferably a substitution, at one or more positions corresponding to positions 2, 3, 5, 6, 7, 9, 10, 11, 12, 13, 14, 15, 16, 17, 21, 22, 23, 24, 25, 26, 27, 28, 29, 31, 32, 33, 34, 35, 36, 38, 39, 40, 41, 42, 43, 44, 45, 47, 49, 51, 55, 57, 58, 59, 60, 61, 62, 63, 64, 66, 68, 70, 71, 73, 74, 75, 76, 77, 78, 79, 80, 81, 83, 85, 86, 87, 88, 89, 90, 91, 92, 93, 94, 95, 96, 97, 98, 100, 101, 102, 106, 107, 108, 109, 110, 111, 112, 113, 114, 115, 116, 117, 118, 119, 120, 121, 122, 123, 124, 125, 128, 129, 130, 132, 136, 137, 138, 139, 140, 141, 142, 146, 148, 150, 153, 154, 155, 157, 158, 159, 161, 162, 164, 165, 166, 167, 169, 170, 171, 172, 173, 174, 176, 177, 178, 179, 180, 181, 182, 183, 184, 185, 186, 187, 188, 189, 190, 193, 194, 195, 196, 197, 198, 199, 201, 202, 203, 204, 206, 207, 208, 209, 210, 211, 212, 213, 214, 215, 217, 218, 220, 221, 222, 223, 226, 228, 229, 230, 231, 232, 233, 234, 235, 236, 237, 238, 239, 240, 241, 242, 243, 244, 245, 246, 247, 248, 249, 250, 251, 252, 254, 255, 256, 257, 258, 259, 260, 261, 262, 265, 267, 268, 269, 272, 273, 275, 277, 278, 279, 280, 281, 282, 283, 284, 285, 286, 288, 291, 292, 293, 294, 295, 296, 297, 298, 301, 302, 303, 304, 305, 306, 307, 308, 310, 311, 312, 314, 315, 316, 317, 318, 320, 321, 322, 324, 325, 326, 328, 329, 331, 335, 337, 338, 340, 341, 342, 343, 344, 345, 346, 347, 348, 349, 350, 351, 352, 353, 354, 355, 357, 359, 360, 362, 363, 364, 365, 366, 367, 368, 369, 370, 371, 372, 373, 374, 375, 376, 377, 379, 380, 381, 383, 386, 387, 388, 389, 390, 391, 392, 393, 394, 395, 396, 398, 399, 400, 401, 402, 403, 404, 406, 407, 408, 409, 410, 411, 413, 414, 415, 416, 417, 418, 419, 421, 422, 423, 424, 426, 427, 428, 429, 430, 431, 432, 433, 434, 435, 436, 437, 439, 440, 441, 442, 443, 444, 445, 446, 448, 449, 450, 451, 452, 453, 454, 455, 456, 457, 459, 461, 462, 463, 464, 465, 466, 467, 469, 470, 471, 472, 474, 475, 477, 478, 479, 480, 485, 487, 488, 491, 492, 493, 494, 495, 496, 497, 498, 499, 500, 501, 502, 503, 504, 505, 506, 507, 508, 509, 510, 511, 512, 514, 516, 517, 518, 519, 520, 521, 524, 525, 526, 527, 528, 529, 530, 531, 534, 535, 537, 538, 539, 540, 542, 543, 545, 546, 547, 548, 549, 550, 551, 553, 554, 555, 556, 557, 558, 559, 561, 563, 564, 565, 567, 570, 572, 573, 574, 575, 576, 577, 578, 579, 580, 581, 582, 583, 584, 585, 586, 587, 588, 589, 590, 591, 592, 593, 594, 595, 597, 598, 600, 604, 606, 608, 609, 610, 611, 612, 631, 634, 635, 637, 638, 639, 644, 645, 646, 647, 648, 650, 651, 652, 653, 654, 655, 656, 657, 659, 661, 662, 669, 670, 673, 674, 675, 676, 677, 678, 679, 680, 681, 682, 683, 684, 686, 687, 688, 689, 690, 694, 695, 696, 697, 698, 700, 701, 704, 705, 706, 708, 709, 710, 711, 712, 713, 714, 715, 718, 720, 721, 723, 726, 727, 728, 729, 731, 732, 733, 734, 735, 737, 738, 739, 740, 741, 742, 744, 745, 747, 750, 751, 752, 756, 757, 759, 760, 766, 769, 770, 771, 772, 773, 774, 775, 776, 777, 778, 780, 781, 782, 783, 784, 785, 786, 787, 788, 789, 790, 791, 792, 793, 794, 795, 796, 798, 799, 800, 801, 802, 803, 804, 805, 806, 807, 808, 809, 810, 811, 812, 813, 814, 815, 816, 817, 818, 819, 820, 821, 822, 825, 826, 827, 828, 829, 830, 831, 832, 833, 834, 835, 836, 837, 838, 840, 841, 842, 843, 844, 845, 846, 849, 850, 853, 854, 857, 858, 859, 860, 861, 862, 863, 864, 865, 866, 870, 871, 872, 873, 874, 877, 880, 881, 882, 883, 884, 885, 886, 887, 888, 889, 893, 894, 896, 897, 899, 900, 901, 902, 903, 904, 905, 907, 908, 910, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 965, 966, 967, 968, 969, 970, 973, 974, 975, 976, 977, 978, 979, 980, 981, 985, 986, 987, 988, 989, 990, 991, 992, 994, 995, 996, 997, 998, 999, 1000, 1002, 1003, 1004, 1005, 1006, 1007, 1009, 1010, 1011, 1012, 1013, 1014, 1015, 1016, 1018, 1021, 1022, 1023, 1024, 1026, 1027, 1029, 1030, 1031, 1034, 1035, 1036, 1037, 1041, 1042, 1043, 1044, 1046, 1048, 1050, 1051, 1052, 1053, 1055, 1057, 1058, 1059, 1060, 1062, 1063, 1064, 1065, 1066, 1067, 1068, 1070, 1071, 1072, 1073, 1074, 1075, 1076, 1078, 1082, 1083, 1084, 1085, 1086, 1087, 1088, 1089, 1090, 1091, 1092, 1093, 1094, 1095, 1096, 1097, 1098, 1099, 1100, 1101, 1102, 1103, 1104, 1105, 1106, 1107, 1108, 1109, 1111, 1113, 1114, 1115, 1116, 1117, 1118, 1119, 1120, 1121, 1122, 1123, 1124, 1125, 1126, 1127, 1128, 1129, 1130, 1131, 1132, 1133, 1134, 1135, 1136, 1137, 1138, 1140, 1141, 1142, 1143, 1144, 1145, 1146, 1147, 1148, 1149, 1150, 1151, 1152, 1153, 1154, 1155, 1158, 1159, 1161, 1162, 1167, 1168, 1169, 1170, 1171, 1172, 1173, 1174, 1175, 1177, 1178, 1179, 1180, 1182, 1184, 1186, 1188, 1189, 1190, 1191, 1192, 1197, 1198, 1199, 1202, 1203, 1204, 1205, 1208, 1209, 1210, 1211, 1213, 1214, 1215, 1217, 1218, 1219, 1220, 1221, 1222, 1223, 1224, 1225, 1226, 1227, 1228, 1230, 1231, 1232, 1233, 1234, 1235, 1236, 1237, 1238, 1239, 1240, 1241, 1242, 1243, 1244, 1246, 1247, 1248, 1249, 1250, 1251, 1252, 1254, 1255, 1256, 1257, 1258, 1259, 1261, 1262, 1263, 1264, 1265, 1266, 1267, 1269, 1270, 1271, 1272, 1273, 1274, 1275, 1277, 1278, 1279, 1280, 1281, 1282, 1283, 1284, 1285, 1286, 1287, 1288, 1289, 1290, 1291, 1292, 1293, 1294, 1295, 1296, 1297, 1302, 1303 or 1304 of the polypeptide of SEQ ID NO: 1.

12. The lactase variant of the preceding embodiment, wherein the variant has a decreased or an increased galactose inhibition, preferably measured as a galactose inhibition improvement factor (GI-IF) of at least 1.2 or at most 0.8, compared to the lactase of SEQ ID NO: 1.

13. The lactase variant of embodiment 1 comprising an alteration, preferably a substitution, at one or more positions corresponding to positions 1, 2, 3, 5, 7, 9, 10, 13, 14, 15, 16, 17, 19, 20, 21, 22, 24, 25, 26, 27, 28, 29, 30, 31, 32, 34, 35, 36, 38, 39, 40, 41, 42, 43, 45, 47, 49, 51, 55, 57, 58, 59, 60, 61, 62, 63, 64, 66, 70, 73, 74, 75, 76, 77, 78, 79, 83, 85, 86, 87, 88, 89, 90, 91, 92, 93, 94, 95, 96, 97, 98, 100, 101, 102, 106, 107, 108, 109, 110, 111, 114, 115, 116, 118, 120, 121, 124, 130, 132, 137, 138, 140, 141, 142, 149, 150, 154, 157, 158, 159, 161, 162, 164, 165, 166, 167, 170, 171, 172, 173, 174, 176, 177, 178, 179, 180, 181, 183, 184, 185, 186, 187, 188, 190, 193, 194, 195, 196, 197, 199, 201, 202, 215, 217, 218, 220, 222, 223, 226, 228, 231, 234, 235, 236, 237, 242, 243, 247, 251, 252, 257, 261, 266, 267, 268, 270, 272, 277, 281, 282, 286, 291, 292, 293, 294, 295, 296, 297, 298, 301, 302, 303, 304, 305, 307, 309, 310, 312, 314, 315, 316, 317, 318, 320, 321, 322, 324, 325, 326, 328, 331, 335, 337, 338, 340, 341, 342, 343, 344, 345, 346, 347, 348, 349, 350, 352, 353, 354, 357, 359, 360, 362, 363, 364, 365, 366, 367, 368, 369, 370, 371, 372, 373, 375, 376, 377, 379, 380, 381, 383, 386, 387, 388, 389, 390, 393, 395, 396, 398, 399, 400, 401, 402, 403, 404, 405, 406, 407, 410, 411, 414, 415, 417, 422, 426, 427, 429, 430, 431, 432, 433, 434, 435, 436, 439, 442, 443, 444, 446, 448, 449, 450, 451, 452, 453, 454, 455, 456, 457, 458, 459, 460, 461, 462, 464, 465, 466, 467, 468, 471, 472, 474, 475, 477, 478, 479, 485, 487, 488, 493, 494, 497, 499, 505, 506, 507, 508, 509, 510, 511, 512, 513, 515, 516, 517, 518, 519, 520, 521, 524, 526, 527, 528, 529, 535, 537, 538, 540, 549, 553, 556, 558, 559, 564, 565, 567, 570, 572, 575, 580, 581, 582, 583, 584, 586, 589, 591, 592, 597, 598, 600, 604, 607, 608, 609, 610, 614, 619, 625, 627, 628, 629, 630, 631, 632, 634, 637, 639, 644, 645, 646, 647, 648, 651, 652, 653, 654, 655, 657, 661, 662, 667, 669, 673, 674, 675, 676, 679, 680, 687, 688, 689, 690, 691, 692, 694, 695, 696, 697, 698, 701, 705, 706, 708, 710, 711, 712, 713, 714, 715, 716, 717, 718, 719, 720, 721, 722, 723, 726, 727, 728, 729, 731, 732, 733, 734, 735, 737, 738, 739, 740, 741, 744, 745, 747, 750, 751, 752, 754, 756, 757, 759, 766, 769, 770, 771, 772, 773, 774, 776, 777, 778, 781, 782, 783, 785, 786, 789, 790, 791, 792, 798, 799, 800, 802, 803, 805, 806, 808, 809, 810, 811, 813, 814, 815, 816, 818, 819, 820, 821, 822, 825, 826, 829, 831, 832, 833, 834, 835, 836, 838, 839, 840, 844, 853, 854, 858, 861, 865, 866, 867, 870, 872, 873, 874, 882, 883, 885, 886, 887, 888, 889, 892, 893, 894, 897, 900, 901, 902, 903, 904, 905, 906, 907, 908, 910, 912, 914, 915, 916, 917, 918, 919, 921, 922, 923, 925, 926, 927, 928, 929, 930, 931, 933, 934, 935, 936, 937, 938, 939, 940, 941, 942, 943, 946, 947, 948, 949, 951, 953, 954, 955, 956, 957, 959, 960, 961, 962, 965, 966, 967, 968, 969, 970, 971, 973, 974, 976, 977, 978, 980, 981, 985, 986, 987, 989, 990, 991, 992, 995, 997, 998, 1001, 1002, 1003, 1005, 1006, 1007, 1010, 1011, 1012, 1013, 1014, 1016, 1018, 1021, 1022, 1024, 1026, 1029, 1030, 1031, 1034, 1035, 1036, 1041, 1042, 1043, 1044, 1046, 1048, 1051, 1052, 1053, 1055, 1057, 1058, 1060, 1061, 1062, 1064, 1065, 1066, 1067, 1068, 1072, 1074, 1075, 1076, 1082, 1083, 1084, 1085, 1086, 1087, 1088, 1089, 1091, 1093, 1094, 1095, 1096, 1097, 1098, 1099, 1100, 1101, 1102, 1103, 1105, 1109, 1111, 1113, 1114, 1115, 1116, 1117, 1118, 1119, 1121, 1123, 1124, 1125, 1127, 1128, 1129, 1130, 1133, 1135, 1136, 1137, 1138, 1140, 1141, 1142, 1143, 1144, 1145, 1146, 1148, 1149, 1150, 1151, 1152, 1153, 1154, 1158, 1159, 1161, 1162, 1165, 1167, 1169, 1170, 1175, 1178, 1179, 1180, 1182, 1183, 1184, 1186, 1188, 1189, 1190, 1191, 1197, 1200, 1203, 1204, 1205, 1208, 1209, 1210, 1211, 1213, 1214, 1215, 1216, 1217, 1218, 1219, 1220, 1221, 1222, 1224, 1225, 1226, 1227, 1229, 1230, 1232, 1235, 1236, 1238, 1239, 1240, 1247, 1255, 1259, 1262, 1263, 1264, 1265, 1273, 1274, 1275, 1277, 1278, 1279, 1280, 1281, 1284, 1286, 1287, 1288, 1293, 1294, 1295, 1296, 1302, 1303 or 1304 of the polypeptide of SEQ ID NO: 1.

14. The lactase variant of the preceding embodiment, wherein the variant has a decreased galactose inhibition, preferably measured as a galactose inhibition improvement factor (GI-IF) of at least 1.2, compared to the lactase of SEQ ID NO: 1.

15. The lactase variant of embodiment 1 comprising an alteration, preferably a substitution, at one or more positions corresponding to positions 2, 3, 5, 7, 9, 10, 13, 14, 15, 16, 17, 21, 22, 24, 25, 26, 27, 28, 29, 31, 32, 34, 35, 36, 38, 39, 40, 41, 42, 43, 45, 47, 49, 51, 55, 57, 58, 59, 60, 61, 62, 63, 64, 66, 70, 73, 74, 75, 76, 77, 78, 79, 83, 85, 86, 87, 88, 89, 90, 91, 92, 93, 94, 95, 96, 97, 98, 100, 101, 102, 106, 107, 108, 109, 110, 111, 114, 115, 116, 118, 120, 121, 124, 130, 132, 137, 138, 140, 141, 142, 150, 154, 157, 158, 159, 161, 162, 164, 165, 166, 167, 170, 171, 172, 173, 174, 176, 177, 178, 179, 180, 181, 183, 184, 185, 186, 187, 188, 190, 193, 194, 195, 196, 197, 199, 201, 202, 215, 217, 218, 220, 222, 223, 226, 228, 231, 234, 235, 236, 237, 242, 243, 247, 251, 252, 257, 261, 267, 268, 272, 277, 281, 282, 286, 291, 292, 293, 294, 295, 296, 297, 298, 301, 302, 303, 304, 305, 307, 310, 312, 314, 315, 316, 317, 318, 320, 321, 322, 324, 325, 326, 328, 331, 335, 337, 338, 340, 341, 342, 343, 344, 345, 346, 347, 348, 349, 350, 352, 353, 354, 357, 359, 360, 362, 363, 364, 365, 366, 367, 368, 369, 370, 371, 372, 373, 375, 376, 377, 379, 380, 381, 383, 386, 387, 388, 389, 390, 393, 395, 396, 398, 399, 400, 401, 402, 403, 404, 406, 407, 410, 411, 414, 415, 417, 422, 426, 427, 429, 430, 431, 432, 433, 434, 435, 436, 439, 442, 443, 444, 446, 448, 449, 450, 451, 452, 453, 454, 455, 456, 457, 459, 461, 462, 464, 465, 466, 467, 471, 472, 474, 475, 477, 478, 479, 485, 487, 488, 493, 494, 497, 499, 505, 506, 507, 508, 509, 510, 511, 512, 516, 517, 518, 519, 520, 521, 524, 526, 527, 528, 529, 535, 537, 538, 540, 549, 553, 556, 558, 559, 564, 565, 567, 570, 572, 575, 580, 581, 582, 583, 584, 586, 589, 591, 592, 597, 598, 600, 604, 608, 609, 610, 631, 634, 637, 639, 644, 645, 646, 647, 648, 651, 652, 653, 654, 655, 657, 661, 662, 669, 673, 674, 675, 676, 679, 680, 687, 688, 689, 690, 694, 695, 696, 697, 698, 701, 705, 706, 708, 710, 711, 712, 713, 714, 715, 718, 720, 721, 723, 726, 727, 728, 729, 731, 732, 733, 734, 735, 737, 738, 739, 740, 741, 744, 745, 747, 750, 751, 752, 756, 757, 759, 766, 769, 770, 771, 772, 773, 774, 776, 777, 778, 781, 782, 783, 785, 786, 789, 790, 791, 792, 798, 799, 800, 802, 803, 805, 806, 808, 809, 810, 811, 813, 814, 815, 816, 818, 819, 820, 821, 822, 825, 826, 829, 831, 832, 833, 834, 835, 836, 838, 840, 844, 853, 854, 858, 861, 865, 866, 870, 872, 873, 874, 882, 883, 885, 886, 887, 888, 889, 893, 894, 897, 900, 901, 902, 903, 904, 905, 907, 908, 910, 912, 914, 915, 916, 917, 918, 919, 921, 922, 923, 925, 926, 927, 928, 929, 930, 931, 933, 934, 935, 936, 937, 938, 939, 940, 941, 942, 943, 946, 947, 948, 949, 951, 953, 954, 955, 956, 957, 959, 960, 961, 962, 965, 966, 967, 968, 969, 970, 973, 974, 976, 977, 978, 980, 981, 985, 986, 987, 989, 990, 991, 992, 995, 997, 998, 1002, 1003, 1005, 1006, 1007, 1010, 1011, 1012, 1013, 1014, 1016, 1018, 1021, 1022, 1024, 1026, 1029, 1030, 1031, 1034, 1035, 1036, 1041, 1042, 1043, 1044, 1046, 1048, 1051, 1052, 1053, 1055, 1057, 1058, 1060, 1062, 1064, 1065, 1066, 1067, 1068, 1072, 1074, 1075, 1076, 1082, 1083, 1084, 1085, 1086, 1087, 1088, 1089, 1091, 1093, 1094, 1095, 1096, 1097, 1098, 1099, 1100, 1101, 1102, 1103, 1105, 1109, 1111, 1113, 1114, 1115, 1116, 1117, 1118, 1119, 1121, 1123, 1124, 1125, 1127, 1128, 1129, 1130, 1133, 1135, 1136, 1137, 1138, 1140, 1141, 1142, 1143, 1144, 1145, 1146, 1148, 1149, 1150, 1151, 1152, 1153, 1154, 1158, 1159, 1161, 1162, 1167, 1169, 1170, 1175, 1178, 1179, 1180, 1182, 1184, 1186, 1188, 1189, 1190, 1191, 1197, 1203, 1204, 1205, 1208, 1209, 1210, 1211, 1213, 1214, 1215, 1217, 1218, 1219, 1220, 1221, 1222, 1224, 1225, 1226, 1227, 1230, 1232, 1235, 1236, 1238, 1239, 1240, 1247, 1255, 1259, 1262, 1263, 1264, 1265, 1273, 1274, 1275, 1277, 1278, 1279, 1280, 1281, 1284, 1286, 1287, 1288, 1293, 1294, 1295, 1296, 1302, 1303 or 1304 of the polypeptide of SEQ ID NO: 1.

16. The lactase variant of the preceding embodiment, wherein the variant has a decreased galactose inhibition, preferably measured as a galactose inhibition improvement factor (GI-IF) of at least 1.2, compared to the lactase of SEQ ID NO: 1.

17. The lactase variant of embodiment 1 comprising an alteration, preferably a substitution, at one or more positions corresponding to positions 1, 2, 3, 6, 7, 13, 14, 15, 16, 17, 19, 20, 21, 22, 23, 24, 25, 26, 27, 28, 29, 31, 32, 33, 34, 35, 39, 40, 41, 42, 43, 45, 47, 49, 51, 53, 55, 57, 58, 59, 60, 62, 64, 66, 68, 73, 75, 76, 77, 78, 79, 81, 83, 85, 86, 87, 88, 90, 91, 92, 93, 94, 95, 96, 97, 98, 100, 101, 102, 106, 107, 108, 109, 110, 111, 112, 114, 115, 116, 118, 120, 121, 122, 123, 124, 130, 132, 136, 137, 142, 146, 149, 150, 153, 154, 157, 158, 159, 161, 162, 164, 165, 166, 167, 169, 170, 172, 173, 174, 176, 177, 178, 179, 180, 181, 184, 185, 187, 188, 189, 190, 194, 195, 197, 198, 199, 201, 202, 203, 204, 205, 206, 207, 209, 211, 212, 213, 214, 215, 217, 218, 220, 221, 222, 223, 228, 229, 230, 231, 232, 233, 234, 235, 236, 237, 238, 239, 240, 242, 243, 245, 247, 248, 249, 251, 252, 254, 255, 256, 258, 261, 262, 264, 266, 267, 268, 272, 278, 279, 280, 281, 282, 284, 285, 286, 291, 292, 293, 295, 296, 298, 301, 303, 304, 305, 308, 309, 310, 311, 312, 314, 315, 318, 320, 321, 322, 324, 325, 326, 328, 331, 335, 337, 340, 341, 342, 343, 344, 345, 346, 347, 348, 350, 352, 353, 357, 359, 360, 362, 363, 364, 365, 366, 368, 369, 370, 371, 372, 373, 376, 377, 379, 380, 381, 383, 386, 387, 388, 389, 390, 391, 392, 393, 394, 395, 396, 398, 399, 400, 401, 402, 403, 404, 405, 406, 407, 408, 409, 410, 411, 413, 414, 415, 416, 417, 418, 419, 420, 421, 422, 423, 424, 426, 427, 429, 430, 431, 433, 434, 435, 436, 437, 439, 440, 441, 442, 443, 444, 445, 446, 448, 449, 450, 451, 452, 454, 455, 456, 457, 458, 459, 461, 462, 463, 464, 465, 466, 468, 470, 471, 472, 474, 475, 477, 479, 480, 485, 487, 488, 491, 492, 493, 494, 495, 497, 498, 499, 500, 501, 504, 505, 506, 507, 508, 509, 510, 511, 512, 513, 514, 515, 516, 517, 518, 519, 520, 521, 522, 524, 525, 526, 527, 528, 530, 531, 534, 535, 537, 540, 542, 543, 545, 546, 547, 548, 549, 550, 551, 553, 554, 555, 556, 558, 559, 561, 563, 564, 565, 567, 570, 572, 573, 574, 575, 576, 577, 578, 579, 580, 581, 582, 583, 584, 585, 586, 587, 588, 589, 590, 591, 592, 594, 595, 598, 600, 606, 607, 608, 609, 610, 611, 614, 619, 620, 625, 627, 628, 630, 631, 634, 635, 639, 644, 650, 655, 656, 657, 661, 667, 669, 670, 673, 674, 675, 676, 677, 678, 679, 680, 681, 684, 687, 688, 689, 690, 692, 694, 695, 697, 698, 700, 701, 704, 705, 708, 709, 710, 711, 712, 714, 716, 717, 718, 719, 721, 723, 726, 727, 728, 731, 732, 734, 735, 738, 739, 740, 741, 744, 745, 747, 750, 752, 754, 756, 757, 760, 769, 770, 771, 772, 773, 774, 775, 776, 777, 778, 781, 782, 783, 784, 785, 786, 787, 788, 789, 790, 792, 793, 794, 795, 796, 798, 799, 800, 802, 803, 804, 806, 807, 808, 810, 812, 813, 814, 815, 816, 818, 819, 820, 821, 822, 826, 828, 829, 830, 831, 832, 833, 835, 836, 837, 838, 839, 840, 841, 844, 845, 849, 850, 854, 857, 858, 859, 860, 861, 862, 863, 865, 866, 867, 870, 872, 873, 874, 877, 881, 883, 884, 886, 887, 888, 892, 893, 896, 897, 900, 901, 902, 903, 904, 905, 906, 907, 908, 910, 912, 914, 915, 916, 917, 918, 919, 920, 921, 922, 923, 926, 927, 929, 933, 934, 935, 936, 937, 938, 939, 940, 941, 942, 943, 944, 946, 947, 948, 950, 951, 953, 955, 956, 958, 961, 962, 965, 966, 967, 968, 969, 970, 971, 973, 974, 976, 977, 978, 979, 980, 981, 985, 986, 987, 990, 991, 995, 997, 999, 1000, 1001, 1002, 1003, 1004, 1005, 1006, 1007, 1011, 1012, 1014, 1018, 1021, 1022, 1024, 1026, 1030, 1035, 1041, 1042, 1048, 1050, 1051, 1052, 1053, 1057, 1058, 1059, 1061, 1062, 1064, 1065, 1066, 1067, 1068, 1070, 1071, 1072, 1073, 1074, 1075, 1082, 1083, 1084, 1085, 1088, 1089, 1091, 1092, 1093, 1094, 1097, 1098, 1099, 1100, 1101, 1102, 1103, 1104, 1106, 1107, 1108, 1109, 1111, 1113, 1114, 1115, 1116, 1117, 1119, 1121, 1125, 1126, 1127, 1128, 1129, 1130, 1133, 1134, 1135, 1136, 1137, 1140, 1141, 1142, 1144, 1145, 1148, 1149, 1150, 1151, 1152, 1153, 1154, 1162, 1165, 1167, 1168, 1170, 1171, 1175, 1177, 1178, 1179, 1180, 1184, 1186, 1188, 1190, 1191, 1192, 1197, 1199, 1200, 1202, 1204, 1208, 1209, 1210, 1211, 1214, 1215, 1216, 1217, 1218, 1219, 1220, 1222, 1223, 1224, 1225, 1226, 1228, 1229, 1230, 1231, 1232, 1233, 1235, 1236, 1237, 1238, 1240, 1242, 1243, 1244, 1246, 1248, 1249, 1250, 1252, 1255, 1258, 1259, 1262, 1263, 1265, 1269, 1274, 1275, 1277, 1278, 1279, 1280, 1281, 1284, 1286, 1287, 1288, 1291, 1292, 1295, 1296, 1302 or 1304 of the polypeptide of SEQ ID NO: 1.

18. The lactase variant of the preceding embodiment, wherein the variant has a decreased or an increased galactose inhibition, preferably measured as a galactose inhibition improvement factor (GI-IF) of at least 1.3 or at most 0.7, compared to the lactase of SEQ ID NO: 1.

19. The lactase variant of embodiment 1 comprising an alteration, preferably a substitution, at one or more positions corresponding to positions 2, 3, 6, 7, 13, 14, 15, 16, 17, 21, 22, 23, 24, 25, 26, 27, 28, 29, 31, 32, 33, 34, 35, 39, 40, 41, 42, 43, 45, 47, 49, 51, 55, 57, 58, 59, 60, 62, 64, 66, 68, 73, 75, 76, 77, 78, 79, 81, 83, 85, 86, 87, 88, 90, 91, 92, 93, 94, 95, 96, 97, 98, 100, 101, 102, 106, 107, 108, 109, 110, 111, 112, 114, 115, 116, 118, 120, 121, 122, 123, 124, 130, 132, 136, 137, 142, 146, 150, 153, 154, 157, 158, 159, 161, 162, 164, 165, 166, 167, 169, 170, 172, 173, 174, 176, 177, 178, 179, 180, 181, 184, 185, 187, 188, 189, 190, 194, 195, 197, 198, 199, 201, 202, 203, 204, 206, 207, 209, 211, 212, 213, 214, 215, 217, 218, 220, 221, 222, 223, 228, 229, 230, 231, 232, 233, 234, 235, 236, 237, 238, 239, 240, 242, 243, 245, 247, 248, 249, 251, 252, 254, 255, 256, 258, 261, 262, 267, 268, 272, 278, 279, 280, 281, 282, 284, 285, 286, 291, 292, 293, 295, 296, 298, 301, 303, 304, 305, 308, 310, 311, 312, 314, 315, 318, 320, 321, 322, 324, 325, 326, 328, 331, 335, 337, 340, 341, 342, 343, 344, 345, 346, 347, 348, 350, 352, 353, 357, 359, 360, 362, 363, 364, 365, 366, 368, 369, 370, 371, 372, 373, 376, 377, 379, 380, 381, 383, 386, 387, 388, 389, 390, 391, 392, 393, 394, 395, 396, 398, 399, 400, 401, 402, 403, 404, 406, 407, 408, 409, 410, 411, 413, 414, 415, 416, 417, 418, 419, 421, 422, 423, 424, 426, 427, 429, 430, 431, 433, 434, 435, 436, 437, 439, 440, 441, 442, 443, 444, 445, 446, 448, 449, 450, 451, 452, 454, 455, 456, 457, 459, 461, 462, 463, 464, 465, 466, 470, 471, 472, 474, 475, 477, 479, 480, 485, 487, 488, 491, 492, 493, 494, 495, 497, 498, 499, 500, 501, 504, 505, 506, 507, 508, 509, 510, 511, 512, 514, 516, 517, 518, 519, 520, 521, 524, 525, 526, 527, 528, 530, 531, 534, 535, 537, 540, 542, 543, 545, 546, 547, 548, 549, 550, 551, 553, 554, 555, 556, 558, 559, 561, 563, 564, 565, 567, 570, 572, 573, 574, 575, 576, 577, 578, 579, 580, 581, 582, 583, 584, 585, 586, 587, 588, 589, 590, 591, 592, 594, 595, 598, 600, 606, 608, 609, 610, 611, 631, 634, 635, 639, 644, 650, 655, 656, 657, 661, 669, 670, 673, 674, 675, 676, 677, 678, 679, 680, 681, 684, 687, 688, 689, 690, 694, 695, 697, 698, 700, 701, 704, 705, 708, 709, 710, 711, 712, 714, 718, 721, 723, 726, 727, 728, 731, 732, 734, 735, 738, 739, 740, 741, 744, 745, 747, 750, 752, 756, 757, 760, 769, 770, 771, 772, 773, 774, 775, 776, 777, 778, 781, 782, 783, 784, 785, 786, 787, 788, 789, 790, 792, 793, 794, 795, 796, 798, 799, 800, 802, 803, 804, 806, 807, 808, 810, 812, 813, 814, 815, 816, 818, 819, 820, 821, 822, 826, 828, 829, 830, 831, 832, 833, 835, 836, 837, 838, 840, 841, 844, 845, 849, 850, 854, 857, 858, 859, 860, 861, 862, 863, 865, 866, 870, 872, 873, 874, 877, 881, 883, 884, 886, 887, 888, 893, 896, 897, 900, 901, 902, 903, 904, 905, 907, 908, 910, 912, 914, 915, 916, 917, 918, 919, 920, 921, 922, 923, 926, 927, 929, 933, 934, 935, 936, 937, 938, 939, 940, 941, 942, 943, 944, 946, 947, 948, 950, 951, 953, 955, 956, 958, 961, 962, 965, 966, 967, 968, 969, 970, 973, 974, 976, 977, 978, 979, 980, 981, 985, 986, 987, 990, 991, 995, 997, 999, 1000, 1002, 1003, 1004, 1005, 1006, 1007, 1011, 1012, 1014, 1018, 1021, 1022, 1024, 1026, 1030, 1035, 1041, 1042, 1048, 1050, 1051, 1052, 1053, 1057, 1058, 1059, 1062, 1064, 1065, 1066, 1067, 1068, 1070, 1071, 1072, 1073, 1074, 1075, 1082, 1083, 1084, 1085, 1088, 1089, 1091, 1092, 1093, 1094, 1097, 1098, 1099, 1100, 1101, 1102, 1103, 1104, 1106, 1107, 1108, 1109, 1111, 1113, 1114, 1115, 1116, 1117, 1119, 1121, 1125, 1126, 1127, 1128, 1129, 1130, 1133, 1134, 1135, 1136, 1137, 1140, 1141, 1142, 1144, 1145, 1148, 1149, 1150, 1151, 1152, 1153, 1154, 1162, 1167, 1168, 1170, 1171, 1175, 1177, 1178, 1179, 1180, 1184, 1186, 1188, 1190, 1191, 1192, 1197, 1199, 1202, 1204, 1208, 1209, 1210, 1211, 1214, 1215, 1217, 1218, 1219, 1220, 1222, 1223, 1224, 1225, 1226, 1228, 1230, 1231, 1232, 1233, 1235, 1236, 1237, 1238, 1240, 1242, 1243, 1244, 1246, 1248, 1249, 1250, 1252, 1255, 1258, 1259, 1262, 1263, 1265, 1269, 1274, 1275, 1277, 1278, 1279, 1280, 1281, 1284, 1286, 1287, 1288, 1291, 1292, 1295, 1296, 1302 or 1304 of the polypeptide of SEQ ID NO: 1.

20. The lactase variant of the preceding embodiment, wherein the variant has a decreased or an increased galactose inhibition, preferably measured as a galactose inhibition improvement factor (GI-IF) of at least 1.3 or at most 0.7, compared to the lactase of SEQ ID NO: 1.

21. The lactase variant of embodiment 1 comprising an alteration, preferably a substitution, at one or more positions corresponding to positions 1, 2, 3, 7, 13, 14, 15, 16, 17, 20, 21, 22, 24, 25, 26, 27, 28, 29, 31, 32, 35, 39, 40, 41, 42, 43, 45, 47, 49, 51, 55, 57, 58, 59, 60, 62, 64, 66, 73, 75, 76, 77, 78, 79, 83, 85, 86, 87, 88, 90, 91, 92, 93, 94, 95, 96, 97, 98, 100, 101, 102, 106, 107, 108, 109, 110, 111, 114, 115, 116, 118, 120, 121, 130, 132, 137, 142, 149, 150, 154, 157, 158, 159, 161, 162, 164, 165, 166, 167, 170, 172, 173, 174, 176, 177, 178, 179, 180, 181, 184, 185, 187, 188, 190, 194, 197, 199, 201, 202, 217, 218, 220, 222, 223, 228, 236, 237, 242, 251, 252, 261, 266, 267, 268, 272, 281, 291, 292, 293, 295, 296, 298, 301, 303, 304, 305, 309, 310, 312, 314, 315, 318, 320, 321, 322, 324, 325, 326, 328, 331, 335, 337, 340, 341, 342, 343, 344, 345, 346, 347, 348, 350, 352, 353, 357, 359, 360, 362, 363, 364, 365, 366, 368, 369, 370, 371, 372, 373, 376, 377, 379, 380, 381, 383, 386, 387, 388, 389, 390, 393, 395, 396, 398, 399, 400, 401, 402, 403, 404, 405, 406, 407, 410, 411, 414, 417, 422, 427, 429, 431, 433, 434, 435, 436, 439, 442, 443, 444, 446, 448, 449, 450, 451, 452, 454, 455, 456, 457, 458, 459, 461, 462, 465, 466, 468, 471, 472, 475, 477, 479, 485, 487, 493, 494, 497, 499, 505, 506, 507, 508, 509, 510, 511, 512, 513, 515, 516, 517, 518, 519, 520, 521, 524, 526, 527, 528, 537, 540, 549, 558, 559, 564, 565, 567, 570, 575, 582, 583, 591, 592, 598, 600, 607, 614, 619, 625, 627, 628, 630, 631, 634, 639, 655, 657, 661, 667, 669, 673, 674, 676, 679, 680, 687, 688, 689, 690, 692, 694, 695, 697, 698, 701, 705, 708, 710, 712, 714, 716, 717, 718, 719, 721, 723, 726, 727, 728, 731, 732, 734, 735, 738, 739, 740, 741, 744, 745, 750, 752, 756, 757, 769, 770, 771, 772, 773, 774, 776, 777, 778, 781, 782, 783, 785, 786, 789, 790, 798, 799, 802, 803, 806, 808, 810, 813, 818, 819, 821, 822, 826, 829, 831, 832, 835, 836, 838, 839, 840, 854, 858, 861, 865, 866, 867, 870, 872, 873, 874, 883, 886, 887, 888, 892, 893, 897, 900, 901, 902, 903, 904, 905, 906, 907, 908, 910, 912, 914, 915, 916, 917, 918, 919, 921, 922, 923, 926, 927, 929, 933, 934, 935, 936, 937, 938, 939, 940, 941, 942, 943, 946, 947, 948, 951, 953, 955, 956, 961, 962, 965, 966, 967, 968, 969, 970, 971, 973, 974, 976, 977, 978, 980, 981, 985, 986, 987, 990, 991, 995, 997, 1001, 1002, 1003, 1005, 1006, 1007, 1011, 1012, 1014, 1018, 1022, 1024, 1026, 1030, 1035, 1041, 1042, 1048, 1051, 1052, 1053, 1057, 1061, 1062, 1064, 1065, 1066, 1067, 1068, 1072, 1074, 1082, 1083, 1084, 1085, 1088, 1089, 1091, 1093, 1094, 1098, 1099, 1101, 1102, 1103, 1109, 1111, 1113, 1114, 1115, 1116, 1117, 1119, 1121, 1125, 1127, 1128, 1129, 1130, 1133, 1135, 1136, 1137, 1140, 1141, 1142, 1144, 1145, 1148, 1149, 1150, 1151, 1152, 1153, 1162, 1167, 1178, 1179, 1180, 1184, 1186, 1188, 1190, 1191, 1197, 1204, 1208, 1209, 1210, 1211, 1214, 1215, 1216, 1217, 1218, 1222, 1224, 1225, 1226, 1229, 1230, 1232, 1235, 1236, 1238, 1255, 1259, 1262, 1263, 1265, 1274, 1275, 1277, 1278, 1279, 1280, 1281, 1284, 1286, 1287, 1288, 1295, 1296, 1302 or 1304 of the polypeptide of SEQ ID NO: 1.

22. The lactase variant of the preceding embodiment, wherein the variant has a decreased galactose inhibition, preferably measured as a galactose inhibition improvement factor (GI-IF) of at least 1.3, compared to the lactase of SEQ ID NO: 1.

23. The lactase variant of embodiment 1 comprising an alteration, preferably a substitution, at one or more positions corresponding to positions 2, 3, 7, 13, 14, 15, 16, 17, 21, 22, 24, 25, 26, 27, 28, 29, 31, 32, 35, 39, 40, 41, 42, 43, 45, 47, 49, 51, 55, 57, 58, 59, 60, 62, 64, 66, 73, 75, 76, 77, 78, 79, 83, 85, 86, 87, 88, 90, 91, 92, 93, 94, 95, 96, 97, 98, 100, 101, 102, 106, 107, 108, 109, 110, 111, 114, 115, 116, 118, 120, 121, 130, 132, 137, 142, 150, 154, 157, 158, 159, 161, 162, 164, 165, 166, 167, 170, 172, 173, 174, 176, 177, 178, 179, 180, 181, 184, 185, 187, 188, 190, 194, 197, 199, 201, 202, 217, 218, 220, 222, 223, 228, 236, 237, 242, 251, 252, 261, 267, 268, 272, 281, 291, 292, 293, 295, 296, 298, 301, 303, 304, 305, 310, 312, 314, 315, 318, 320, 321, 322, 324, 325, 326, 328, 331, 335, 337, 340, 341, 342, 343, 344, 345, 346, 347, 348, 350, 352, 353, 357, 359, 360, 362, 363, 364, 365, 366, 368, 369, 370, 371, 372, 373, 376, 377, 379, 380, 381, 383, 386, 387, 388, 389, 390, 393, 395, 396, 398, 399, 400, 401, 402, 403, 404, 406, 407, 410, 411, 414, 417, 422, 427, 429, 431, 433, 434, 435, 436, 439, 442, 443, 444, 446, 448, 449, 450, 451, 452, 454, 455, 456, 457, 459, 461, 462, 465, 466, 471, 472, 475, 477, 479, 485, 487, 493, 494, 497, 499, 505, 506, 507, 508, 509, 510, 511, 512, 516, 517, 518, 519, 520, 521, 524, 526, 527, 528, 537, 540, 549, 558, 559, 564, 565, 567, 570, 575, 582, 583, 591, 592, 598, 600, 631, 634, 639, 655, 657, 661, 669, 673, 674, 676, 679, 680, 687, 688, 689, 690, 694, 695, 697, 698, 701, 705, 708, 710, 712, 714, 718, 721, 723, 726, 727, 728, 731, 732, 734, 735, 738, 739, 740, 741, 744, 745, 750, 752, 756, 757, 769, 770, 771, 772, 773, 774, 776, 777, 778, 781, 782, 783, 785, 786, 789, 790, 798, 799, 802, 803, 806, 808, 810, 813, 818, 819, 821, 822, 826, 829, 831, 832, 835, 836, 838, 840, 854, 858, 861, 865, 866, 870, 872, 873, 874, 883, 886, 887, 888, 893, 897, 900, 901, 902, 903, 904, 905, 907, 908, 910, 912, 914, 915, 916, 917, 918, 919, 921, 922, 923, 926, 927, 929, 933, 934, 935, 936, 937, 938, 939, 940, 941, 942, 943, 946, 947, 948, 951, 953, 955, 956, 961, 962, 965, 966, 967, 968, 969, 970, 973, 974, 976, 977, 978, 980, 981, 985, 986, 987, 990, 991, 995, 997, 1002, 1003, 1005, 1006, 1007, 1011, 1012, 1014, 1018, 1022, 1024, 1026, 1030, 1035, 1041, 1042, 1048, 1051, 1052, 1053, 1057, 1062, 1064, 1065, 1066, 1067, 1068, 1072, 1074, 1082, 1083, 1084, 1085, 1088, 1089, 1091, 1093, 1094, 1098, 1099, 1101, 1102, 1103, 1109, 1111, 1113, 1114, 1115, 1116, 1117, 1119, 1121, 1125, 1127, 1128, 1129, 1130, 1133, 1135, 1136, 1137, 1140, 1141, 1142, 1144, 1145, 1148, 1149, 1150, 1151, 1152, 1153, 1162, 1167, 1178, 1179, 1180, 1184, 1186, 1188, 1190, 1191, 1197, 1204, 1208, 1209, 1210, 1211, 1214, 1215, 1217, 1218, 1222, 1224, 1225, 1226, 1230, 1232, 1235, 1236, 1238, 1255, 1259, 1262, 1263, 1265, 1274, 1275, 1277, 1278, 1279, 1280, 1281, 1284, 1286, 1287, 1288, 1295, 1296, 1302 or 1304 of the polypeptide of SEQ ID NO: 1.

24. The lactase variant of the preceding embodiment, wherein the variant has a decreased galactose inhibition, preferably measured as a galactose inhibition improvement factor (GI-IF) of at least 1.3, compared to the lactase of SEQ ID NO: 1.

25. The lactase variant of embodiment 1 comprising an alteration, preferably a substitution, at one or more positions corresponding to positions 2, 3, 7, 13, 15, 16, 20, 21, 23, 24, 26, 27, 28, 29, 31, 32, 34, 35, 40, 42, 43, 45, 47, 49, 51, 53, 55, 57, 58, 59, 62, 64, 66, 73, 75, 76, 77, 78, 79, 81, 85, 86, 87, 88, 90, 91, 92, 93, 94, 95, 96, 97, 98, 100, 101, 102, 109, 110, 111, 112, 114, 115, 120, 121, 124, 130, 132, 136, 137, 146, 149, 150, 153, 157, 158, 159, 161, 162, 165, 166, 167, 170, 172, 174, 176, 177, 178, 179, 180, 185, 188, 189, 190, 197, 217, 218, 220, 222, 223, 228, 233, 236, 237, 239, 242, 247, 248, 249, 251, 252, 254, 255, 256, 261, 262, 264, 268, 272, 278, 279, 286, 291, 292, 293, 296, 298, 301, 303, 304, 305, 309, 312, 320, 325, 326, 328, 337, 340, 341, 342, 343, 345, 348, 350, 359, 360, 362, 363, 364, 365, 368, 369, 370, 373, 376, 377, 379, 380, 381, 386, 387, 388, 389, 390, 391, 392, 393, 394, 395, 396, 398, 399, 400, 401, 402, 403, 404, 405, 406, 408, 409, 411, 413, 414, 416, 417, 418, 419, 420, 423, 424, 426, 427, 429, 431, 433, 434, 435, 436, 439, 442, 443, 444, 445, 448, 449, 450, 451, 452, 454, 455, 456, 457, 458, 459, 465, 466, 468, 472, 475, 477, 479, 485, 487, 488, 491, 493, 495, 498, 499, 500, 504, 505, 506, 507, 508, 509, 510, 511, 512, 513, 514, 515, 516, 517, 518, 519, 520, 521, 522, 524, 525, 526, 527, 528, 531, 537, 540, 546, 547, 548, 549, 550, 551, 553, 554, 555, 556, 558, 559, 564, 565, 567, 570, 572, 574, 576, 577, 579, 582, 583, 585, 586, 588, 592, 594, 595, 600, 607, 608, 609, 610, 611, 614, 619, 620, 625, 630, 634, 635, 639, 644, 650, 655, 656, 661, 669, 670, 673, 676, 680, 684, 687, 688, 692, 694, 695, 697, 698, 700, 701, 705, 709, 710, 712, 714, 716, 717, 718, 719, 721, 723, 726, 728, 731, 734, 735, 739, 740, 741, 745, 750, 752, 754, 756, 757, 760, 769, 770, 772, 773, 774, 775, 776, 777, 781, 782, 783, 784, 785, 786, 787, 789, 792, 793, 794, 795, 798, 799, 800, 803, 806, 808, 810, 813, 814, 815, 816, 818, 821, 822, 826, 830, 831, 832, 835, 836, 838, 839, 840, 844, 850, 854, 858, 863, 865, 867, 870, 872, 874, 883, 887, 888, 892, 893, 901, 902, 903, 904, 905, 906, 908, 910, 912, 914, 915, 916, 917, 918, 919, 921, 922, 923, 927, 934, 935, 936, 937, 938, 940, 942, 943, 944, 946, 947, 948, 950, 956, 961, 962, 965, 966, 968, 969, 973, 976, 977, 978, 979, 980, 981, 985, 986, 987, 990, 995, 997, 1001, 1002, 1003, 1004, 1006, 1007, 1018, 1022, 1024, 1026, 1030, 1035, 1048, 1050, 1051, 1052, 1053, 1057, 1066, 1068, 1071, 1072, 1082, 1083, 1084, 1085, 1088, 1089, 1091, 1092, 1093, 1094, 1097, 1099, 1102, 1104, 1107, 1109, 1111, 1113, 1114, 1115, 1116, 1117, 1119, 1125, 1127, 1128, 1129, 1130, 1133, 1136, 1137, 1141, 1144, 1145, 1150, 1151, 1167, 1175, 1178, 1179, 1180, 1184, 1186, 1188, 1199, 1208, 1209, 1210, 1211, 1214, 1215, 1216, 1217, 1218, 1219, 1222, 1223, 1224, 1225, 1226, 1228, 1229, 1230, 1231, 1233, 1238, 1243, 1244, 1246, 1250, 1259, 1265, 1269, 1274, 1275, 1277, 1278, 1279, 1280, 1281, 1284, 1286, 1287, 1288, 1302 or 1304 of the polypeptide of SEQ ID NO: 1.

26. The lactase variant of the preceding embodiment, wherein the variant has a decreased or an increased galactose inhibition, preferably measured as a galactose inhibition improvement factor (GI-IF) of at least 1.4 or at most 0.6, compared to the lactase of SEQ ID NO: 1.

27. The lactase variant of embodiment 1 comprising an alteration, preferably a substitution, at one or more positions corresponding to positions 2, 3, 7, 13, 15, 16, 21, 23, 24, 26, 27, 28, 29, 31, 32, 34, 35, 40, 42, 43, 45, 47, 49, 51, 55, 57, 58, 59, 62, 64, 66, 73, 75, 76, 77, 78, 79, 81, 85, 86, 87, 88, 90, 91, 92, 93, 94, 95, 96, 97, 98, 100, 101, 102, 109, 110, 111, 112, 114, 115, 120, 121, 124, 130, 132, 136, 137, 146, 150, 153, 157, 158, 159, 161, 162, 165, 166, 167, 170, 172, 174, 176, 177, 178, 179, 180, 185, 188, 189, 190, 197, 217, 218, 220, 222, 223, 228, 233, 236, 237, 239, 242, 247, 248, 249, 251, 252, 254, 255, 256, 261, 262, 268, 272, 278, 279, 286, 291, 292, 293, 296, 298, 301, 303, 304, 305, 312, 320, 325, 326, 328, 337, 340, 341, 342, 343, 345, 348, 350, 359, 360, 362, 363, 364, 365, 368, 369, 370, 373, 376, 377, 379, 380, 381, 386, 387, 388, 389, 390, 391, 392, 393, 394, 395, 396, 398, 399, 400, 401, 402, 403, 404, 406, 408, 409, 411, 413, 414, 416, 417, 418, 419, 423, 424, 426, 427, 429, 431, 433, 434, 435, 436, 439, 442, 443, 444, 445, 448, 449, 450, 451, 452, 454, 455, 456, 457, 459, 465, 466, 472, 475, 477, 479, 485, 487, 488, 491, 493, 495, 498, 499, 500, 504, 505, 506, 507, 508, 509, 510, 511, 512, 514, 516, 517, 518, 519, 520, 521, 524, 525, 526, 527, 528, 531, 537, 540, 546, 547, 548, 549, 550, 551, 553, 554, 555, 556, 558, 559, 564, 565, 567, 570, 572, 574, 576, 577, 579, 582, 583, 585, 586, 588, 592, 594, 595, 600, 608, 609, 610, 611, 634, 635, 639, 644, 650, 655, 656, 661, 669, 670, 673, 676, 680, 684, 687, 688, 694, 695, 697, 698, 700, 701, 705, 709, 710, 712, 714, 718, 721, 723, 726, 728, 731, 734, 735, 739, 740, 741, 745, 750, 752, 756, 757, 760, 769, 770, 772, 773, 774, 775, 776, 777, 781, 782, 783, 784, 785, 786, 787, 789, 792, 793, 794, 795, 798, 799, 800, 803, 806, 808, 810, 813, 814, 815, 816, 818, 821, 822, 826, 830, 831, 832, 835, 836, 838, 840, 844, 850, 854, 858, 863, 865, 870, 872, 874, 883, 887, 888, 893, 901, 902, 903, 904, 905, 908, 910, 912, 914, 915, 916, 917, 918, 919, 921, 922, 923, 927, 934, 935, 936, 937, 938, 940, 942, 943, 944, 946, 947, 948, 950, 956, 961, 962, 965, 966, 968, 969, 973, 976, 977, 978, 979, 980, 981, 985, 986, 987, 990, 995, 997, 1002, 1003, 1004, 1006, 1007, 1018, 1022, 1024, 1026, 1030, 1035, 1048, 1050, 1051, 1052, 1053, 1057, 1066, 1068, 1071, 1072, 1082, 1083, 1084, 1085, 1088, 1089, 1091, 1092, 1093, 1094, 1097, 1099, 1102, 1104, 1107, 1109, 1111, 1113, 1114, 1115, 1116, 1117, 1119, 1125, 1127, 1128, 1129, 1130, 1133, 1136, 1137, 1141, 1144, 1145, 1150, 1151, 1167, 1175, 1178, 1179, 1180, 1184, 1186, 1188, 1199, 1208, 1209, 1210, 1211, 1214, 1215, 1217, 1218, 1219, 1222, 1223, 1224, 1225, 1226, 1228, 1230, 1231, 1233, 1238, 1243, 1244, 1246, 1250, 1259, 1265, 1269, 1274, 1275, 1277, 1278, 1279, 1280, 1281, 1284, 1286, 1287, 1288, 1302 or 1304 of the polypeptide of SEQ ID NO: 1.

28. The lactase variant of the preceding embodiment, wherein the variant has a decreased or an increased galactose inhibition, preferably measured as a galactose inhibition improvement factor (GI-IF) of at least 1.4 or at most 0.6, compared to the lactase of SEQ ID NO: 1.

29. The lactase variant of embodiment 1 comprising an alteration, preferably a substitution, at one or more positions corresponding to positions 2, 3, 7, 13, 15, 16, 20, 21, 24, 26, 27, 28, 29, 31, 32, 40, 45, 47, 49, 51, 55, 57, 58, 59, 62, 64, 73, 75, 76, 77, 78, 79, 85, 86, 87, 88, 90, 91, 92, 93, 94, 95, 96, 97, 98, 100, 101, 102, 109, 110, 111, 114, 115, 120, 121, 130, 132, 137, 149, 150, 157, 158, 159, 161, 162, 165, 166, 167, 170, 172, 174, 176, 177, 178, 179, 180, 185, 188, 197, 217, 218, 220, 222, 223, 228, 236, 237, 251, 252, 268, 272, 291, 292, 293, 296, 298, 301, 303, 304, 305, 309, 312, 320, 325, 326, 328, 337, 340, 341, 343, 345, 348, 350, 359, 360, 362, 363, 364, 365, 368, 369, 370, 373, 376, 377, 379, 380, 381, 386, 388, 389, 390, 393, 395, 396, 399, 400, 401, 402, 403, 404, 405, 406, 411, 417, 427, 429, 431, 433, 434, 435, 436, 439, 442, 443, 444, 448, 449, 450, 451, 452, 454, 455, 456, 457, 458, 459, 465, 466, 468, 472, 475, 477, 479, 485, 487, 493, 499, 505, 506, 507, 508, 509, 510, 511, 512, 513, 515, 516, 517, 518, 519, 520, 521, 524, 526, 527, 528, 537, 540, 549, 558, 559, 564, 565, 567, 582, 583, 592, 600, 607, 614, 619, 625, 634, 639, 655, 661, 669, 673, 676, 680, 687, 688, 692, 694, 695, 697, 698, 701, 705, 710, 712, 714, 716, 717, 718, 719, 721, 723, 726, 728, 734, 735, 739, 740, 741, 750, 752, 756, 757, 769, 770, 772, 773, 774, 777, 781, 782, 785, 786, 789, 798, 799, 803, 806, 810, 818, 821, 822, 831, 832, 835, 836, 839, 840, 854, 858, 865, 867, 872, 874, 883, 887, 888, 892, 893, 901, 902, 903, 904, 905, 906, 908, 910, 912, 914, 915, 916, 917, 918, 919, 921, 922, 923, 934, 935, 936, 937, 938, 942, 943, 946, 947, 956, 961, 962, 965, 966, 968, 969, 973, 976, 977, 978, 980, 981, 985, 986, 987, 990, 995, 997, 1001, 1002, 1003, 1006, 1007, 1018, 1022, 1024, 1030, 1035, 1048, 1051, 1052, 1053, 1057, 1082, 1083, 1084, 1085, 1088, 1089, 1093, 1094, 1099, 1102, 1109, 1111, 1113, 1114, 1115, 1116, 1117, 1119, 1125, 1127, 1128, 1129, 1130, 1133, 1137, 1144, 1145, 1150, 1151, 1167, 1178, 1179, 1180, 1184, 1186, 1188, 1208, 1209, 1210, 1211, 1214, 1215, 1216, 1217, 1218, 1222, 1224, 1225, 1226, 1229, 1230, 1238, 1259, 1265, 1274, 1275, 1277, 1278, 1279, 1280, 1281, 1284, 1286, 1287, 1288, 1302 or 1304 of the polypeptide of SEQ ID NO: 1.

30. The lactase variant of the preceding embodiment, wherein the variant has a decreased galactose inhibition, preferably measured as a galactose inhibition improvement factor (GI-IF) of at least 1.4, compared to the lactase of SEQ ID NO: 1.

31. The lactase variant of embodiment 1 comprising an alteration, preferably a substitution, at one or more positions corresponding to positions 2, 3, 7, 13, 15, 16, 21, 24, 26, 27, 28, 29, 31, 32, 40, 45, 47, 49, 51, 55, 57, 58, 59, 62, 64, 73, 75, 76, 77, 78, 79, 85, 86, 87, 88, 90, 91, 92, 93, 94, 95, 96, 97, 98, 100, 101, 102, 109, 110, 111, 114, 115, 120, 121, 130, 132, 137, 150, 157, 158, 159, 161, 162, 165, 166, 167, 170, 172, 174, 176, 177, 178, 179, 180, 185, 188, 197, 217, 218, 220, 222, 223, 228, 236, 237, 251, 252, 268, 272, 291, 292, 293, 296, 298, 301, 303, 304, 305, 312, 320, 325, 326, 328, 337, 340, 341, 343, 345, 348, 350, 359, 360, 362, 363, 364, 365, 368, 369, 370, 373, 376, 377, 379, 380, 381, 386, 388, 389, 390, 393, 395, 396, 399, 400, 401, 402, 403, 404, 406, 411, 417, 427, 429, 431, 433, 434, 435, 436, 439, 442, 443, 444, 448, 449, 450, 451, 452, 454, 455, 456, 457, 459, 465, 466, 472, 475, 477, 479, 485, 487, 493, 499, 505, 506, 507, 508, 509, 510, 511, 512, 516, 517, 518, 519, 520, 521, 524, 526, 527, 528, 537, 540, 549, 558, 559, 564, 565, 567, 582, 583, 592, 600, 634, 639, 655, 661, 669, 673, 676, 680, 687, 688, 694, 695, 697, 698, 701, 705, 710, 712, 714, 718, 721, 723, 726, 728, 734, 735, 739, 740, 741, 750, 752, 756, 757, 769, 770, 772, 773, 774, 777, 781, 782, 785, 786, 789, 798, 799, 803, 806, 810, 818, 821, 822, 831, 832, 835, 836, 840, 854, 858, 865, 872, 874, 883, 887, 888, 893, 901, 902, 903, 904, 905, 908, 910, 912, 914, 915, 916, 917, 918, 919, 921, 922, 923, 934, 935, 936, 937, 938, 942, 943, 946, 947, 956, 961, 962, 965, 966, 968, 969, 973, 976, 977, 978, 980, 981, 985, 986, 987, 990, 995, 997, 1002, 1003, 1006, 1007, 1018, 1022, 1024, 1030, 1035, 1048, 1051, 1052, 1053, 1057, 1082, 1083, 1084, 1085, 1088, 1089, 1093, 1094, 1099, 1102, 1109, 1111, 1113, 1114, 1115, 1116, 1117, 1119, 1125, 1127, 1128, 1129, 1130, 1133, 1137, 1144, 1145, 1150, 1151, 1167, 1178, 1179, 1180, 1184, 1186, 1188, 1208, 1209, 1210, 1211, 1214, 1215, 1217, 1218, 1222, 1224, 1225, 1226, 1230, 1238, 1259, 1265, 1274, 1275, 1277, 1278, 1279, 1280, 1281, 1284, 1286, 1287, 1288, 1302 or 1304 of the polypeptide of SEQ ID NO: 1.

32. The lactase variant of the preceding embodiment, wherein the variant has a decreased galactose inhibition, preferably measured as a galactose inhibition improvement factor (GI-IF) of at least 1.4, compared to the lactase of SEQ ID NO: 1.

33. The lactase variant of embodiment 1 comprising an alteration, preferably a substitution, at one or more positions corresponding to positions 7, 15, 16, 20, 24, 27, 28, 29, 31, 32, 45, 47, 49, 51, 55, 57, 58, 59, 66, 75, 76, 77, 81, 85, 86, 87, 88, 90, 92, 93, 94, 95, 96, 98, 101, 102, 109, 112, 120, 121, 124, 137, 149, 153, 158, 159, 161, 162, 165, 166, 167, 170, 172, 174, 178, 185, 188, 189, 217, 218, 220, 222, 223, 236, 237, 239, 247, 251, 252, 254, 264, 268, 272, 279, 291, 298, 301, 303, 304, 305, 326, 337, 341, 345, 348, 359, 362, 363, 364, 368, 369, 370, 373, 376, 381, 386, 387, 389, 390, 391, 392, 393, 394, 395, 396, 398, 399, 400, 401, 402, 403, 404, 405, 406, 413, 414, 416, 417, 418, 420, 423, 424, 427, 429, 431, 433, 434, 435, 436, 439, 442, 444, 445, 448, 449, 450, 451, 452, 455, 456, 457, 458, 459, 465, 466, 468, 472, 479, 487, 488, 491, 493, 499, 500, 504, 505, 506, 507, 508, 509, 510, 511, 512, 513, 515, 516, 517, 518, 521, 522, 524, 526, 527, 537, 540, 546, 548, 549, 550, 551, 554, 555, 556, 558, 564, 565, 567, 570, 574, 582, 583, 586, 592, 594, 595, 600, 608, 609, 614, 625, 630, 639, 644, 650, 655, 656, 661, 669, 670, 673, 688, 694, 695, 697, 698, 700, 701, 709, 710, 714, 717, 718, 719, 721, 728, 731, 734, 735, 739, 741, 745, 750, 752, 754, 756, 757, 769, 770, 772, 775, 776, 777, 781, 783, 784, 785, 786, 787, 792, 793, 794, 795, 799, 803, 810, 814, 815, 816, 818, 821, 822, 826, 830, 835, 836, 839, 844, 850, 854, 865, 867, 872, 883, 893, 901, 902, 904, 912, 914, 915, 916, 917, 918, 919, 921, 922, 923, 934, 935, 936, 937, 943, 944, 946, 956, 961, 965, 968, 969, 973, 981, 985, 986, 1002, 1006, 1007, 1030, 1035, 1048, 1050, 1051, 1053, 1066, 1068, 1084, 1085, 1089, 1093, 1094, 1097, 1102, 1109, 1113, 1114, 1115, 1116, 1117, 1119, 1125, 1127, 1128, 1129, 1130, 1133, 1137, 1144, 1145, 1150, 1151, 1167, 1178, 1179, 1180, 1184, 1208, 1210, 1211, 1214, 1216, 1219, 1222, 1223, 1224, 1225, 1226, 1229, 1230, 1238, 1259, 1265, 1269, 1274, 1275, 1277, 1278, 1279, 1281, 1284, 1286, 1287, 1288, 1302 or 1304 of the polypeptide of SEQ ID NO: 1.

34. The lactase variant of the preceding embodiment, wherein the variant has a decreased or an increased galactose inhibition, preferably measured as a galactose inhibition improvement factor (GI-IF) of at least 1.5 or at most 0.5, compared to the lactase of SEQ ID NO: 1.

35. The lactase variant of embodiment 1 comprising an alteration, preferably a substitution, at one or more positions corresponding to positions 7, 15, 16, 24, 27, 28, 29, 31, 32, 45, 47, 49, 51, 55, 57, 58, 59, 66, 75, 76, 77, 81, 85, 86, 87, 88, 90, 92, 93, 94, 95, 96, 98, 101, 102, 109, 112, 120, 121, 124, 137, 153, 158, 159, 161, 162, 165, 166, 167, 170, 172, 174, 178, 185, 188, 189, 217, 218, 220, 222, 223, 236, 237, 239, 247, 251, 252, 254, 268, 272, 279, 291, 298, 301, 303, 304, 305, 326, 337, 341, 345, 348, 359, 362, 363, 364, 368, 369, 370, 373, 376, 381, 386, 387, 389, 390, 391, 392, 393, 394, 395, 396, 398, 399, 400, 401, 402, 403, 404, 406, 413, 414, 416, 417, 418, 423, 424, 427, 429, 431, 433, 434, 435, 436, 439, 442, 444, 445, 448, 449, 450, 451, 452, 455, 456, 457, 459, 465, 466, 472, 479, 487, 488, 491, 493, 499, 500, 504, 505, 506, 507, 508, 509, 510, 511, 512, 516, 517, 518, 521, 524, 526, 527, 537, 540, 546, 548, 549, 550, 551, 554, 555, 556, 558, 564, 565, 567, 570, 574, 582, 583, 586, 592, 594, 595, 600, 608, 609, 639, 644, 650, 655, 656, 661, 669, 670, 673, 688, 694, 695, 697, 698, 700, 701, 709, 710, 714, 718, 721, 728, 731, 734, 735, 739, 741, 745, 750, 752, 756, 757, 769, 770, 772, 775, 776, 777, 781, 783, 784, 785, 786, 787, 792, 793, 794, 795, 799, 803, 810, 814, 815, 816, 818, 821, 822, 826, 830, 835, 836, 844, 850, 854, 865, 872, 883, 893, 901, 902, 904, 912, 914, 915, 916, 917, 918, 919, 921, 922, 923, 934, 935, 936, 937, 943, 944, 946, 956, 961, 965, 968, 969, 973, 981, 985, 986, 1002, 1006, 1007, 1030, 1035, 1048, 1050, 1051, 1053, 1066, 1068, 1084, 1085, 1089, 1093, 1094, 1097, 1102, 1109, 1113, 1114, 1115, 1116, 1117, 1119, 1125, 1127, 1128, 1129, 1130, 1133, 1137, 1144, 1145, 1150, 1151, 1167, 1178, 1179, 1180, 1184, 1208, 1210, 1211, 1214, 1219, 1222, 1223, 1224, 1225, 1226, 1230, 1238, 1259, 1265, 1269, 1274, 1275, 1277, 1278, 1279, 1281, 1284, 1286, 1287, 1288, 1302 or 1304 of the polypeptide of SEQ ID NO: 1.

36. The lactase variant of the preceding embodiment, wherein the variant has a decreased or an increased galactose inhibition, preferably measured as a galactose inhibition improvement factor (GI-IF) of at least 1.5 or at most 0.5, compared to the lactase of SEQ ID NO: 1.

37. The lactase variant of embodiment 1 comprising an alteration, preferably a substitution, at one or more positions corresponding to positions 7, 15, 16, 20, 24, 27, 28, 29, 31, 32, 45, 47, 49, 51, 55, 57, 58, 59, 75, 76, 77, 85, 86, 87, 88, 90, 92, 93, 94, 95, 96, 98, 101, 102, 109, 120, 121, 137, 149, 158, 159, 161, 162, 165, 166, 167, 170, 172, 174, 178, 185, 188, 217, 218, 220, 222, 223, 236, 237, 251, 252, 268, 272, 291, 298, 301, 303, 304, 326, 337, 341, 345, 348, 359, 362, 363, 364, 368, 369, 370, 373, 376, 381, 386, 389, 390, 393, 395, 396, 399, 400, 401, 402, 403, 404, 405, 406, 417, 427, 429, 431, 433, 434, 435, 436, 439, 442, 444, 448, 449, 450, 451, 452, 455, 456, 457, 458, 459, 465, 466, 468, 472, 479, 487, 499, 505, 506, 507, 508, 509, 510, 511, 512, 513, 515, 516, 517, 518, 521, 524, 526, 527, 537, 540, 549, 558, 564, 565, 567, 582, 583, 600, 614, 625, 639, 655, 661, 669, 673, 688, 694, 695, 697, 698, 701, 710, 714, 717, 718, 719, 721, 728, 735, 739, 741, 750, 752, 756, 757, 769, 770, 772, 777, 781, 785, 786, 799, 803, 810, 818, 821, 822, 835, 836, 839, 854, 865, 867, 872, 883, 893, 901, 902, 904, 912, 914, 915, 916, 917, 918, 919, 921, 922, 923, 934, 935, 936, 937, 943, 956, 961, 965, 968, 969, 973, 981, 985, 986, 1002, 1006, 1007, 1030, 1035, 1048, 1051, 1053, 1084, 1085, 1089, 1093, 1094, 1102, 1109, 1113, 1114, 1115, 1116, 1117, 1119, 1125, 1127, 1128, 1129, 1130, 1133, 1137, 1144, 1145, 1150, 1151, 1167, 1178, 1179, 1180, 1184, 1208, 1210, 1211, 1214, 1216, 1222, 1224, 1225, 1226, 1229, 1230, 1238, 1259, 1265, 1274, 1275, 1277, 1278, 1279, 1281, 1284, 1286, 1287, 1288, 1302 or 1304 of the polypeptide of SEQ ID NO: 1.

38. The lactase variant of the preceding embodiment, wherein the variant has a decreased galactose inhibition, preferably measured as a galactose inhibition improvement factor (GI-IF) of at least 1.5, compared to the lactase of SEQ ID NO: 1.

39. The lactase variant of embodiment 1 comprising an alteration, preferably a substitution, at one or more positions corresponding to positions 7, 15, 16, 24, 27, 28, 29, 31, 32, 45, 47, 49, 51, 55, 57, 58, 59, 75, 76, 77, 85, 86, 87, 88, 90, 92, 93, 94, 95, 96, 98, 101, 102, 109, 120, 121, 137, 158, 159, 161, 162, 165, 166, 167, 170, 172, 174, 178, 185, 188, 217, 218, 220, 222, 223, 236, 237, 251, 252, 268, 272, 291, 298, 301, 303, 304, 326, 337, 341, 345, 348, 359, 362, 363, 364, 368, 369, 370, 373, 376, 381, 386, 389, 390, 393, 395, 396, 399, 400, 401, 402, 403, 404, 406, 417, 427, 429, 431, 433, 434, 435, 436, 439, 442, 444, 448, 449, 450, 451, 452, 455, 456, 457, 459, 465, 466, 472, 479, 487, 499, 505, 506, 507, 508, 509, 510, 511, 512, 516, 517, 518, 521, 524, 526, 527, 537, 540, 549, 558, 564, 565, 567, 582, 583, 600, 639, 655, 661, 669, 673, 688, 694, 695, 697, 698, 701, 710, 714, 718, 721, 728, 735, 739, 741, 750, 752, 756, 757, 769, 770, 772, 777, 781, 785, 786, 799, 803, 810, 818, 821, 822, 835, 836, 854, 865, 872, 883, 893, 901, 902, 904, 912, 914, 915, 916, 917, 918, 919, 921, 922, 923, 934, 935, 936, 937, 943, 956, 961, 965, 968, 969, 973, 981, 985, 986, 1002, 1006, 1007, 1030, 1035, 1048, 1051, 1053, 1084, 1085, 1089, 1093, 1094, 1102, 1109, 1113, 1114, 1115, 1116, 1117, 1119, 1125, 1127, 1128, 1129, 1130, 1133, 1137, 1144, 1145, 1150, 1151, 1167, 1178, 1179, 1180, 1184, 1208, 1210, 1211, 1214, 1222, 1224, 1225, 1226, 1230, 1238, 1259, 1265, 1274, 1275, 1277, 1278, 1279, 1281, 1284, 1286, 1287, 1288, 1302 or 1304 of the polypeptide of SEQ ID NO: 1.

40. The lactase variant of the preceding embodiment, wherein the variant has a decreased galactose inhibition, preferably measured as a galactose inhibition improvement factor (GI-IF) of at least 1.5, compared to the lactase of SEQ ID NO: 1.

41. The lactase variant of embodiment 1 comprising an alteration, preferably a substitution, at one or more positions corresponding to positions 7, 15, 20, 24, 27, 31, 45, 47, 49, 51, 57, 59, 77, 85, 86, 87, 88, 90, 92, 93, 94, 96, 101, 112, 121, 161, 162, 165, 166, 167, 170, 174, 185, 217, 218, 220, 222, 223, 236, 237, 239, 252, 268, 298, 301, 303, 304, 337, 362, 364, 368, 369, 373, 376, 381, 386, 389, 390, 391, 392, 393, 394, 395, 396, 398, 400, 401, 402, 403, 404, 405, 406, 418, 420, 427, 429, 433, 434, 444, 445, 448, 449, 450, 451, 452, 455, 456, 459, 466, 468, 472, 479, 488, 491, 499, 504, 505, 506, 507, 508, 509, 510, 511, 512, 513, 515, 517, 521, 522, 524, 526, 527, 546, 549, 550, 554, 555, 556, 558, 564, 567, 570, 582, 583, 592, 594, 600, 608, 609, 614, 625, 630, 639, 644, 650, 655, 661, 669, 670, 673, 688, 694, 695, 698, 700, 701, 710, 714, 721, 731, 734, 745, 750, 752, 756, 757, 769, 770, 772, 775, 776, 777, 781, 783, 784, 785, 787, 792, 794, 803, 810, 814, 822, 826, 830, 835, 836, 839, 844, 865, 867, 872, 902, 912, 914, 915, 916, 917, 919, 922, 923, 937, 943, 944, 946, 961, 969, 973, 985, 986, 1002, 1006, 1007, 1048, 1051, 1066, 1068, 1084, 1085, 1093, 1094, 1097, 1102, 1109, 1113, 1114, 1116, 1117, 1125, 1127, 1129, 1133, 1137, 1145, 1151, 1167, 1178, 1179, 1180, 1184, 1210, 1211, 1214, 1216, 1222, 1223, 1225, 1226, 1230, 1238, 1265, 1274, 1275, 1277, 1278, 1279, 1281, 1284, 1286, 1287, 1288 or 1302 of the polypeptide of SEQ ID NO: 1.

42. The lactase variant of the preceding embodiment, wherein the variant has a decreased or an increased galactose inhibition, preferably measured as a galactose inhibition improvement factor (GI-IF) of at least 1.6 or at most 0.4, compared to the lactase of SEQ ID NO: 1.

43. The lactase variant of embodiment 1 comprising an alteration, preferably a substitution, at one or more positions corresponding to positions 7, 15, 24, 27, 31, 45, 47, 49, 51, 57, 59, 77, 85, 86, 87, 88, 90, 92, 93, 94, 96, 101, 112, 121, 161, 162, 165, 166, 167, 170, 174, 185, 217, 218, 220, 222, 223, 236, 237, 239, 252, 268, 298, 301, 303, 304, 337, 362, 364, 368, 369, 373, 376, 381, 386, 389, 390, 391, 392, 393, 394, 395, 396, 398, 400, 401, 402, 403, 404, 406, 418, 427, 429, 433, 434, 444, 445, 448, 449, 450, 451, 452, 455, 456, 459, 466, 472, 479, 488, 491, 499, 504, 505, 506, 507, 508, 509, 510, 511, 512, 517, 521, 524, 526, 527, 546, 549, 550, 554, 555, 556, 558, 564, 567, 570, 582, 583, 592, 594, 600, 608, 609, 639, 644, 650, 655, 661, 669, 670, 673, 688, 694, 695, 698, 700, 701, 710, 714, 721, 731, 734, 745, 750, 752, 756, 757, 769, 770, 772, 775, 776, 777, 781, 783, 784, 785, 787, 792, 794, 803, 810, 814, 822, 826, 830, 835, 836, 844, 865, 872, 902, 912, 914, 915, 916, 917, 919, 922, 923, 937, 943, 944, 946, 961, 969, 973, 985, 986, 1002, 1006, 1007, 1048, 1051, 1066, 1068, 1084, 1085, 1093, 1094, 1097, 1102, 1109, 1113, 1114, 1116, 1117, 1125, 1127, 1129, 1133, 1137, 1145, 1151, 1167, 1178, 1179, 1180, 1184, 1210, 1211, 1214, 1222, 1223, 1225, 1226, 1230, 1238, 1265, 1274, 1275, 1277, 1278, 1279, 1281, 1284, 1286, 1287, 1288 or 1302 of the polypeptide of SEQ ID NO: 1.

44. The lactase variant of the preceding embodiment, wherein the variant has a decreased or an increased galactose inhibition, preferably measured as a galactose inhibition improvement factor (GI-IF) of at least 1.6 or at most 0.4, compared to the lactase of SEQ ID NO: 1.

45. The lactase variant of embodiment 1 comprising an alteration, preferably a substitution, at one or more positions corresponding to positions 7, 15, 20, 24, 27, 31, 45, 47, 49, 51, 57, 59, 77, 85, 86, 87, 88, 90, 92, 93, 94, 96, 101, 121, 161, 162, 165, 166, 167, 170, 174, 185, 217, 218, 220, 222, 223, 236, 237, 252, 268, 298, 301, 303, 304, 337, 362, 364, 368, 369, 373, 376, 381, 386, 389, 390, 393, 396, 400, 401, 402, 403, 404, 405, 406, 427, 429, 433, 434, 444, 448, 449, 450, 451, 452, 455, 456, 459, 466, 468, 472, 479, 505, 506, 507, 508, 509, 510, 511, 512, 513, 515, 517, 521, 524, 526, 527, 549, 558, 564, 567, 582, 583, 600, 614, 625, 639, 655, 661, 669, 673, 688, 694, 695, 698, 701, 710, 714, 721, 750, 752, 756, 757, 769, 770, 772, 777, 781, 785, 803, 810, 822, 835, 836, 839, 865, 867, 872, 902, 912, 914, 915, 916, 917, 919, 922, 923, 937, 943, 961, 969, 973, 985, 986, 1002, 1006, 1007, 1048, 1051, 1084, 1085, 1093, 1094, 1102, 1109, 1113, 1114, 1116, 1117, 1125, 1127, 1129, 1133, 1137, 1145, 1167, 1178, 1179, 1180, 1184, 1210, 1211, 1214, 1216, 1222, 1225, 1226, 1230, 1238, 1265, 1274, 1275, 1277, 1278, 1279, 1281, 1284, 1286, 1287, 1288 or 1302 of the polypeptide of SEQ ID NO: 1.

46. The lactase variant of the preceding embodiment, wherein the variant has a decreased galactose inhibition, preferably measured as a galactose inhibition improvement factor (GI-IF) of at least 1.6, compared to the lactase of SEQ ID NO: 1.

47. The lactase variant of embodiment 1 comprising an alteration, preferably a substitution, at one or more positions corresponding to positions 7, 15, 24, 27, 31, 45, 47, 49, 51, 57, 59, 77, 85, 86, 87, 88, 90, 92, 93, 94, 96, 101, 121, 161, 162, 165, 166, 167, 170, 174, 185, 217, 218, 220, 222, 223, 236, 237, 252, 268, 298, 301, 303, 304, 337, 362, 364, 368, 369, 373, 376, 381, 386, 389, 390, 393, 396, 400, 401, 402, 403, 404, 406, 427, 429, 433, 434, 444, 448, 449, 450, 451, 452, 455, 456, 459, 466, 472, 479, 505, 506, 507, 508, 509, 510, 511, 512, 517, 521, 524, 526, 527, 549, 558, 564, 567, 582, 583, 600, 639, 655, 661, 669, 673, 688, 694, 695, 698, 701, 710, 714, 721, 750, 752, 756, 757, 769, 770, 772, 777, 781, 785, 803, 810, 822, 835, 836, 865, 872, 902, 912, 914, 915, 916, 917, 919, 922, 923, 937, 943, 961, 969, 973, 985, 986, 1002, 1006, 1007, 1048, 1051, 1084, 1085, 1093, 1094, 1102, 1109, 1113, 1114, 1116, 1117, 1125, 1127, 1129, 1133, 1137, 1145, 1167, 1178, 1179, 1180, 1184, 1210, 1211, 1214, 1222, 1225, 1226, 1230, 1238, 1265, 1274, 1275, 1277, 1278, 1279, 1281, 1284, 1286, 1287, 1288 or 1302 of the polypeptide of SEQ ID NO: 1.

48. The lactase variant of the preceding embodiment, wherein the variant has a decreased galactose inhibition, preferably measured as a galactose inhibition improvement factor (GI-IF) of at least 1.6, compared to the lactase of SEQ ID NO: 1.

49. The lactase variant of embodiment 1 comprising an alteration, preferably a substitution, at one or more positions corresponding to positions 7, 15, 20, 24, 27, 31, 49, 57, 59, 77, 85, 86, 87, 88, 92, 93, 96, 101, 112, 161, 162, 165, 166, 167, 170, 174, 185, 217, 220, 222, 237, 239, 252, 268, 301, 304, 337, 362, 364, 368, 369, 376, 381, 386, 389, 390, 392, 393, 394, 395, 396, 401, 402, 403, 404, 405, 418, 427, 429, 434, 444, 448, 449, 450, 451, 452, 459, 466, 468, 472, 479, 491, 499, 504, 505, 506, 508, 509, 510, 511, 513, 515, 517, 521, 522, 524, 526, 527, 546, 549, 550, 555, 558, 564, 567, 582, 600, 608, 609, 614, 625, 639, 650, 661, 670, 673, 688, 695, 698, 701, 710, 714, 721, 734, 745, 750, 756, 757, 769, 770, 772, 775, 776, 781, 783, 784, 785, 810, 814, 822, 835, 836, 844, 865, 867, 872, 902, 912, 914, 915, 916, 917, 919, 922, 923, 943, 944, 961, 969, 973, 985, 986, 1002, 1006, 1051, 1084, 1085, 1093, 1109, 1114, 1116, 1117, 1125, 1133, 1178, 1179, 1184, 1210, 1211, 1214, 1216, 1222, 1225, 1226, 1230, 1238, 1265, 1274, 1275, 1277, 1278, 1279, 1281, 1284, 1287, 1288 or 1302 of the polypeptide of SEQ ID NO: 1.

50. The lactase variant of the preceding embodiment, wherein the variant has a decreased or an increased galactose inhibition, preferably measured as a galactose inhibition improvement factor (GI-IF) of at least 1.7 or at most 0.3, compared to the lactase of SEQ ID NO: 1.

51. The lactase variant of embodiment 1 comprising an alteration, preferably a substitution, at one or more positions corresponding to positions 7, 15, 24, 27, 31, 49, 57, 59, 77, 85, 86, 87, 88, 92, 93, 96, 101, 112, 161, 162, 165, 166, 167, 170, 174, 185, 217, 220, 222, 237, 239, 252, 268, 301, 304, 337, 362, 364, 368, 369, 376, 381, 386, 389, 390, 392, 393, 394, 395, 396, 401, 402, 403, 404, 418, 427, 429, 434, 444, 448, 449, 450, 451, 452, 459, 466, 472, 479, 491, 499, 504, 505, 506, 508, 509, 510, 511, 517, 521, 524, 526, 527, 546, 549, 550, 555, 558, 564, 567, 582, 600, 608, 609, 639, 650, 661, 670, 673, 688, 695, 698, 701, 710, 714, 721, 734, 745, 750, 756, 757, 769, 770, 772, 775, 776, 781, 783, 784, 785, 810, 814, 822, 835, 836, 844, 865, 872, 902, 912, 914, 915, 916, 917, 919, 922, 923, 943, 944, 961, 969, 973, 985, 986, 1002, 1006, 1051, 1084, 1085, 1093, 1109, 1114, 1116, 1117, 1125, 1133, 1178, 1179, 1184, 1210, 1211, 1214, 1222, 1225, 1226, 1230, 1238, 1265, 1274, 1275, 1277, 1278, 1279, 1281, 1284, 1287, 1288 or 1302 of the polypeptide of SEQ ID NO: 1.

52. The lactase variant of the preceding embodiment, wherein the variant has a decreased or an increased galactose inhibition, preferably measured as a galactose inhibition improvement factor (GI-IF) of at least 1.7 or at most 0.3, compared to the lactase of SEQ ID NO: 1.

53. The lactase variant of embodiment 1 comprising an alteration, preferably a substitution, at one or more positions corresponding to positions 7, 15, 20, 24, 27, 31, 49, 57, 59, 77, 85, 86, 87, 88, 92, 93, 96, 101, 161, 162, 165, 166, 167, 170, 174, 185, 217, 220, 222, 237, 252, 268, 301, 304, 337, 362, 364, 368, 369, 376, 381, 386, 389, 390, 393, 396, 401, 402, 403, 404, 405, 427, 429, 434, 444, 448, 449, 450, 451, 452, 459, 466, 468, 472, 479, 505, 506, 508, 509, 510, 511, 513, 515, 517, 521, 524, 526, 527, 549, 558, 564, 567, 582, 600, 614, 625, 639, 661, 673, 688, 695, 698, 701, 710, 714, 721, 750, 756, 757, 769, 770, 772, 781, 785, 810, 822, 835, 836, 865, 867, 872, 902, 912, 914, 915, 916, 917, 919, 922, 923, 961, 969, 973, 985, 986, 1002, 1006, 1051, 1084, 1085, 1093, 1109, 1114, 1116, 1117, 1125, 1133, 1178, 1179, 1184, 1210, 1211, 1214, 1216, 1222, 1225, 1226, 1230, 1238, 1265, 1274, 1275, 1277, 1278, 1279, 1281, 1284, 1287, 1288 or 1302 of the polypeptide of SEQ ID NO: 1.

54. The lactase variant of the preceding embodiment, wherein the variant has a decreased galactose inhibition, preferably measured as a galactose inhibition improvement factor (GI-IF) of at least 1.7, compared to the lactase of SEQ ID NO: 1.

55. The lactase variant of embodiment 1 comprising an alteration, preferably a substitution, at one or more positions corresponding to positions 7, 15, 24, 27, 31, 49, 57, 59, 77, 85, 86, 87, 88, 92, 93, 96, 101, 161, 162, 165, 166, 167, 170, 174, 185, 217, 220, 222, 237, 252, 268, 301, 304, 337, 362, 364, 368, 369, 376, 381, 386, 389, 390, 393, 396, 401, 402, 403, 404, 427, 429, 434, 444, 448, 449, 450, 451, 452, 459, 466, 472, 479, 505, 506, 508, 509, 510, 511, 517, 521, 524, 526, 527, 549, 558, 564, 567, 582, 600, 639, 661, 673, 688, 695, 698, 701, 710, 714, 721, 750, 756, 757, 769, 770, 772, 781, 785, 810, 822, 835, 836, 865, 872, 902, 912, 914, 915, 916, 917, 919, 922, 923, 961, 969, 973, 985, 986, 1002, 1006, 1051, 1084, 1085, 1093, 1109, 1114, 1116, 1117, 1125, 1133, 1178, 1179, 1184, 1210, 1211, 1214, 1222, 1225, 1226, 1230, 1238, 1265, 1274, 1275, 1277, 1278, 1279, 1281, 1284, 1287, 1288 or 1302 of the polypeptide of SEQ ID NO: 1.

56. The lactase variant of the preceding embodiment, wherein the variant has a decreased galactose inhibition, preferably measured as a galactose inhibition improvement factor (GI-IF) of at least 1.7, compared to the lactase of SEQ ID NO: 1.

57. The lactase variant of embodiment 1 comprising an alteration, preferably a substitution, at one or more positions corresponding to positions 7, 15, 20, 24, 57, 59, 85, 86, 93, 162, 165, 166, 167, 174, 217, 222, 237, 268, 304, 337, 362, 364, 368, 369, 376, 386, 389, 390, 392, 393, 394, 395, 396, 401, 402, 403, 404, 405, 427, 429, 434, 448, 449, 450, 451, 459, 468, 472, 479, 491, 499, 505, 506, 508, 509, 510, 511, 513, 515, 517, 521, 526, 527, 546, 564, 567, 582, 600, 608, 614, 625, 650, 670, 673, 688, 695, 701, 710, 714, 721, 745, 750, 756, 757, 769, 770, 772, 776, 781, 783, 784, 785, 810, 814, 835, 844, 912, 914, 917, 919, 922, 923, 961, 985, 986, 1006, 1051, 1093, 1116, 1117, 1125, 1178, 1179, 1184, 1210, 1211, 1225, 1238, 1265, 1274, 1275, 1277, 1284, 1287, 1288 or 1302 of the polypeptide of SEQ ID NO: 1.

58. The lactase variant of the preceding embodiment, wherein the variant has a decreased or an increased galactose inhibition, preferably measured as a galactose inhibition improvement factor (GI-IF) of at least 1.8 or at most 0.2, compared to the lactase of SEQ ID NO: 1.

59. The lactase variant of embodiment 1 comprising an alteration, preferably a substitution, at one or more positions corresponding to positions 7, 15, 24, 57, 59, 85, 86, 93, 162, 165, 166, 167, 174, 217, 222, 237, 268, 304, 337, 362, 364, 368, 369, 376, 386, 389, 390, 392, 393, 394, 395, 396, 401, 402, 403, 404, 427, 429, 434, 448, 449, 450, 451, 459, 472, 479, 491, 499, 505, 506, 508, 509, 510, 511, 517, 521, 526, 527, 546, 564, 567, 582, 600, 608, 650, 670, 673, 688, 695, 701, 710, 714, 721, 745, 750, 756, 757, 769, 770, 772, 776, 781, 783, 784, 785, 810, 814, 835, 844, 912, 914, 917, 919, 922, 923, 961, 985, 986, 1006, 1051, 1093, 1116, 1117, 1125, 1178, 1179, 1184, 1210, 1211, 1225, 1238, 1265, 1274, 1275, 1277, 1284, 1287, 1288 or 1302 of the polypeptide of SEQ ID NO: 1.

60. The lactase variant of the preceding embodiment, wherein the variant has a decreased or an increased galactose inhibition, preferably measured as a galactose inhibition improvement factor (GI-IF) of at least 1.8 or at most 0.2, compared to the lactase of SEQ ID NO: 1.

61. The lactase variant of embodiment 1 comprising an alteration, preferably a substitution, at one or more positions corresponding to positions 7, 15, 20, 24, 57, 59, 85, 86, 93, 162, 165, 166, 167, 174, 217, 222, 237, 268, 304, 337, 362, 364, 368, 369, 376, 386, 389, 390, 393, 396, 401, 402, 403, 404, 405, 427, 429, 434, 448, 449, 450, 451, 459, 468, 472, 479, 505, 506, 508, 509, 510, 511, 513, 515, 517, 521, 526, 527, 564, 567, 582, 600, 614, 625, 673, 688, 695, 701, 710, 714, 721, 750, 756, 757, 769, 770, 772, 781, 785, 810, 835, 912, 914, 917, 919, 922, 923, 961, 985, 986, 1006, 1051, 1093, 1116, 1117, 1125, 1178, 1179, 1184, 1210, 1211, 1225, 1238, 1265, 1274, 1275, 1277, 1284, 1287, 1288 or 1302 of the polypeptide of SEQ ID NO: 1.

62. The lactase variant of the preceding embodiment, wherein the variant has a decreased galactose inhibition, preferably measured as a galactose inhibition improvement factor (GI-IF) of at least 1.8, compared to the lactase of SEQ ID NO: 1.

63. The lactase variant of embodiment 1 comprising an alteration, preferably a substitution, at one or more positions corresponding to positions 7, 15, 24, 57, 59, 85, 86, 93, 162, 165, 166, 167, 174, 217, 222, 237, 268, 304, 337, 362, 364, 368, 369, 376, 386, 389, 390, 393, 396, 401, 402, 403, 404, 427, 429, 434, 448, 449, 450, 451, 459, 472, 479, 505, 506, 508, 509, 510, 511, 517, 521, 526, 527, 564, 567, 582, 600, 673, 688, 695, 701, 710, 714, 721, 750, 756, 757, 769, 770, 772, 781, 785, 810, 835, 912, 914, 917, 919, 922, 923, 961, 985, 986, 1006, 1051, 1093, 1116, 1117, 1125, 1178, 1179, 1184, 1210, 1211, 1225, 1238, 1265, 1274, 1275, 1277, 1284, 1287, 1288 or 1302 of the polypeptide of SEQ ID NO: 1.

64. The lactase variant of the preceding embodiment, wherein the variant has a decreased galactose inhibition, preferably measured as a galactose inhibition improvement factor (GI-IF) of at least 1.8, compared to the lactase of SEQ ID NO: 1.

65. The lactase variant of embodiment 1 comprising an alteration, preferably a substitution, at one or more positions corresponding to positions 7, 15, 24, 57, 85, 86, 93, 162, 167, 174, 217, 222, 237, 268, 304, 362, 364, 369, 386, 389, 392, 393, 395, 396, 401, 403, 404, 405, 427, 448, 449, 450, 451, 459, 468, 472, 479, 491, 505, 506, 508, 510, 511, 513, 515, 517, 521, 526, 527, 567, 600, 625, 670, 673, 688, 695, 701, 710, 714, 721, 750, 757, 769, 772, 784, 785, 810, 912, 919, 922, 923, 961, 985, 1006, 1051, 1093, 1116, 1117, 1179, 1210, 1211, 1238, 1265, 1274, 1275, 1277, 1284, 1287, 1288 or 1302 of the polypeptide of SEQ ID NO: 1.

66. The lactase variant of the preceding embodiment, wherein the variant has a decreased or an increased galactose inhibition, preferably measured as a galactose inhibition improvement factor (GI-IF) of at least 1.9 or at most 0.1, compared to the lactase of SEQ ID NO: 1.

67. The lactase variant of embodiment 1 comprising an alteration, preferably a substitution, at one or more positions corresponding to positions 7, 15, 24, 57, 85, 86, 93, 162, 167, 174, 217, 222, 237, 268, 304, 362, 364, 369, 386, 389, 392, 393, 395, 396, 401, 403, 404, 427, 448, 449, 450, 451, 459, 472, 479, 491, 505, 506, 508, 510, 511, 517, 521, 526, 527, 567, 600, 670, 673, 688, 695, 701, 710, 714, 721, 750, 757, 769, 772, 784, 785, 810, 912, 919, 922, 923, 961, 985, 1006, 1051, 1093, 1116, 1117, 1179, 1210, 1211, 1238, 1265, 1274, 1275, 1277, 1284, 1287, 1288 or 1302 of the polypeptide of SEQ ID NO: 1.

68. The lactase variant of the preceding embodiment, wherein the variant has a decreased or an increased galactose inhibition, preferably measured as a galactose inhibition improvement factor (GI-IF) of at least 1.9 or at most 0.1, compared to the lactase of SEQ ID NO: 1.

69. The lactase variant of embodiment 1 comprising an alteration, preferably a substitution, at one or more positions corresponding to positions 7, 15, 24, 57, 85, 86, 93, 162, 167, 174, 217, 222, 237, 268, 304, 362, 364, 369, 386, 389, 393, 396, 401, 403, 404, 405, 427, 448, 449, 450, 451, 459, 468, 472, 479, 505, 506, 508, 510, 511, 513, 515, 517, 521, 526, 527, 567, 600, 625, 673, 688, 695, 701, 710, 714, 721, 750, 757, 769, 772, 785, 810, 912, 919, 922, 923, 961, 985, 1006, 1051, 1093, 1116, 1117, 1179, 1210, 1211, 1238, 1265, 1274, 1275, 1277, 1284, 1287, 1288 or 1302 of the polypeptide of SEQ ID NO: 1.

70. The lactase variant of the preceding embodiment, wherein the variant has a decreased galactose inhibition, preferably measured as a galactose inhibition improvement factor (GI-IF) of at least 1.9, compared to the lactase of SEQ ID NO: 1.

71. The lactase variant of embodiment 1 comprising an alteration, preferably a substitution, at one or more positions corresponding to positions 7, 15, 24, 57, 85, 86, 93, 162, 167, 174, 217, 222, 237, 268, 304, 362, 364, 369, 386, 389, 393, 396, 401, 403, 404, 427, 448, 449, 450, 451, 459, 472, 479, 505, 506, 508, 510, 511, 517, 521, 526, 527, 567, 600, 673, 688, 695, 701, 710, 714, 721, 750, 757, 769, 772, 785, 810, 912, 919, 922, 923, 961, 985, 1006, 1051, 1093, 1116, 1117, 1179, 1210, 1211, 1238, 1265, 1274, 1275, 1277, 1284, 1287, 1288 or 1302 of the polypeptide of SEQ ID NO: 1.

72. The lactase variant of the preceding embodiment, wherein the variant has a decreased galactose inhibition, preferably measured as a galactose inhibition improvement factor (GI-IF) of at least 1.9, compared to the lactase of SEQ ID NO: 1.

73. The lactase variant of any of the preceding embodiments wherein the alteration is a substitution.

74. The lactase variant of embodiment 1 which comprises one or more of the following substitutions, wherein each position corresponds to a position in SEQ ID NO: 1: V1D, V1F, V1H, V1K, V1L, V1R, E2D, E2G, E2Q, E2V, D3A, D3H, D3I, D3N, D3S, D3V, D3W, A4C, A4G, A4H, A4I, A4L, A4M, A4P, T5A, T5D, T5F, T5K, T5R, T5S, T5V, R6A, R6D, R6F, R6G, R6H, R6L, R6M, R6P, R6S, R6W, S7A, S7D, S7I, S7L, S7N, S7P, S7T, S7W, S9G, S9H, S9P, S9R, S9W, T10G, T10K, T10L, T10P, T10R, T10S, T10W, T11L, T11P, Q12L, Q12R, Q12V, Q12Y, M13C, M13D, M13E, M13F, M13H, M13K, M13R, M13W, S14A, S14G, S14H, S14L, S14T, S14V, S14Y, S15C, S15F, S15I, S15K, S15L, S15P, S15R, S15T, S15V, S15W, S15Y, T16A, T16C, T16I, T16N, T16S, P17A, P17C, P17D, P17E, P17I, P17L, P17N, P17R, P17S, P17T, V19A, V19F, V19G, V19I, V19K, V19L, V19N, V19S, V19W, V20C, V20F, V20G, V20I, V20K, V20L, V20M, V20N, V20P, V20Q, V20R, V20T, V20W, Y21A, Y21C, Y21D, Y21F, Y21G, Y21H, Y21M, Y21P, Y21R, Y21T, S22A, S22E, S22F, S22G, S22L, S22M, S22N, S22R, S22T, S22W, S23A, S23C, S23D, S23L, S23M, S23R, A24F, A24G, A24L, A24R, A24T, A24W, V25D, V25E, V25F, V25G, V25H, V25K, V25L, V25M, V25Q, V25R, V25S, V25T, V25W, D26C, D26I, D26L, D26M, D26T, D26V, S27A, S27C, S27F, S27G, S27H, S27P, S27Y, K28C, K28G, K28I, K28L, K28R, K28S, K28V, K28W, Q29D, Q29F, Q29G, Q29L, Q29M, Q29R, Q29S, Q29V, Q29W, N30A, N30G, N30H, N30M, N30P, N30V, N30W, N30Y, R31E, R31G, R31I, R31M, R31V, T32M, T32Q, T32R, T32S, S33C, S33E, S33H, S33K, S33N, S33Q, S33R, S33V, D34C, D34E, D34F, D34G, D34H, D34L, D34S, D34W, D34Y, F35A, F35C, F35E, F35G, F35K, F35N, F35T, F35V, D36H, D36Q, A37N, A37Q, N38C, N38G, N38S, W39G, W39S, K40C, K40D, K40F, K40G, K40I, K40M, K40N, K40P, K40W, F41A, F41C, F41G, F41I, F41Q, F41S, F41Y, M42E, M42N, M42T, L43A, L43C, L43G, L43I, L43S, L43T, L43V, S44C, S44M, S44N, S44Y, D45A, D45L, D45P, D45V, V47K, V47R, Q48S, A49C, A49D, A49H, A49R, A49S, A49T, A49V, D51G, D51I, D51K, D51M, D51P, D51V, A53C, A53G, A53L, A53R, A53S, A53T, A53V, A53W, F54M, F54S, D55C, D55F, D55G, D55H, D55M, D55N, D55P, D55S, D55V, S57A, S57C, S57E, S57G, A58D, A58G, A58I, A58M, A58N, A58Q, A58R, A58T, W59D, W59I, W59K, W59L, W59N, W59P, W59V, Q60A, Q60C, Q60E, Q60F, Q60G, Q60K, Q60L, Q60M, Q60R, Q60S, Q60V, Q60Y, Q61K, Q61P, Q61S, V62G, V62N, V62S, V62T, V62W, D63G, D63L, D63P, D63S, D63V, L64E, L64G, H66C, H66L, H66R, H66T, H66W, H66Y, D67E, Y68P, Y68V, I70A, I70H, I70K, I70P, I70R, T71C, T71E, T71G, T71H, T71K, T71L, T71N, T71P, T71Q, T71R, T71S, K73A, K73D, K73F, K73G, K73Q, K73V, Y74G, Y74K, Y74T, Y74W, S75G, S75L, S75Q, S75R, S75V, Q76G, Q76I, Q76K, Q76M, Q76P, Q76S, Q76V, Q76Y, S77C, S77D, S77E, S77F, S77G, S77H, S77I, S77K, S77L, S77M, S77R, S77T, S77V, S77W, S77Y, N78C, N78E, N78F, N78K, N78Q, N78R, N78S, N78T, E79H, E79Q, E79S, E79T, E79W, A80K, E81A, E81Q, A83E, A83T, L85A, L85C, L85D, L85F, L85M, L85N, L85S, L85V, L85W, P86E, P86G, P86H, P86N, P86Q, P86R, P86V, P86W, P86Y, G87A, G87D, G87E, G87N, G87Q, G88A, G88F, G88I, G88M, G88Q, G88S, T89C, T89G, T89H, T89K, T89L, T89M, T89N, T89P, T89W, T89Y, G90A, G90C, G90D, G90L, G90S, G90T, G90V, W91E, W91L, W91P, W91Q, W91R, W91S, W91Y, Y92F, Y92H, Y92I, Y92M, Y92N, Y92S, Y92T, Y92V, Y92W, R93A, R93D, R93F, R93H, R93I, R93L, R93N, R93T, R93V, R93Y, K94A, K94C, K94G, K94R, K94S, K94T, K94V, S95A, S95C, S95D, S95E, S95G, S95I, S95L, S95Q, S95R, F96A, F96C, F96I, F96K, F96L, F96M, F96P, F96S, F96V, F96W, T97F, T97S, T97V, I98C, I98H, I98S, I98W, R100T, D101A, D101P, D101V, L102A, L102G, L102M, L102P, L102S, G104C, K105D, K105Q, K105R, K105W, K105Y, R106K, R106P, R106V, R106W, I107A, I107F, I107G, I107Q, I107S, A108E, A108S, A108V, I109M, I109T, N110A, N110F, N110S, N110T, N110V, N110W, F111A, F111C, F111L, F111Q, F111V, D112A, D112F, D112G, D112T, G113A, G113S, V114F, V114G, V114M, V114R, Y115E, M116A, M116C, M116D, M116W, M116Y, N117K, N117R, N117T, N117W, A118K, A118P, A118Y, T119A, T119G, T119L, V120A, V120K, W121C, W121D, W121R, W121T, W121V, W121Y, F122A, F122M, F122S, F122Y, N123P, G124E, G124M, G124Q, G124R, V125D, V125E, V125I, K126E, K126V, G128A, G128D, T129E, T129V, H130A, H130C, H130Q, H130S, H130T, P131K, P131L, P131S, Y132C, Y132E, Y132S, G133E, S135E, S135P, S135V, P136R, P136Y, F137A, F137C, F137D, F137G, F137L, F137P, S138A, S138D, S138G, S138H, S138L, S138M, S138R, S138V, F139A, F139E, F139Q, F139W, D140G, D140L, D140V, L141G, L141T, T142E, T142S, T142V, K146A, G148H, G148K, G148T, G149E, G149H, G149I, G149M, G149Q, G149Y, E150A, E150C, E150G, E150L, E150N, E150R, N151L, I153A, I153Y, V154E, V154I, V154K, V154L, V154M, V154S, V155F, V157A, V157G, V157L, V157P, V157Q, V157S, E158G, E158H, E158K, E158Q, E158V, N159D, N159H, N159S, N159T, R160G, L161E, L161K, L161M, L161S, L161W, P162F, P162G, P162N, P162T, P162W, S164A, R165H, W166S, Y167A, Y167C, S168C, G169A, G169C, G169D, G169S, S170L, S170Q, G171C, G171F, G171T, I172G, I172K, I172P, I172Q, Y173A, Y173H, Y173M, Y173P, Y173S, Y173W, R174E, R174K, D175E, D175Y, V176E, V176K, V176T, T177A, T177C, T177E, T177K, T177L, L178I, L178Q, L178W, T179A, T179C, T179D, T179H, T179I, T179K, T179L, T179N, T179P, T179S, V180A, V180C, V180D, V180E, V180G, V180M, T181A, T181D, T181F, T181K, T181R, D182F, D182L, D182S, G183W, V184F, V184H, V184P, V184Q, V184R, V184S, V184W, H185G, H185L, H185R, V186A, V186E, V186G, V186N, G187A, G187D, G187H, N188E, N188R, N188S, N188V, N188W, N189A, N189E, G190C, G190F, G190H, G190Q, G190V, V191C, V191T, V191W, V191Y, I193N, I193Q, I193T, I193V, K194A, K194I, K194L, K194R, T195A, T195E, T195M, T195S, T195W, P196A, P196H, P196I, P196M, P196S, P196W, S197A, S197C, S197E, S197K, S197L, S197P, L198E, L198F, L198H, L198I, L198K, L198R, L198V, L198W, A199E, A199F, A199K, A199P, A199R, A199T, Q201C, Q201E, Q201I, Q201K, Q201M, Q201V, N202A, N202D, N202F, N202G, N202K, N202L, N202M, N202Q, N202R, N202S, N202T, N202W, G203C, G203K, G203M, G203Q, G203R, G203S, G203V, G203W, G203Y, G204A, G204C, G204D, G204K, G204R, G204S, G204Y, N205E, N205G, N205H, N205L, N205P, N205W, N205Y, V206A, V206C, V206D, V206F, V206G, V206I, V206K, V206Q, V206R, V206S, V206T, T207A, T207C, T207G, T207I, T207K, T207L, T207M, T207N, T207Q, T207R, T207W, M208A, M208S, M208T, N209C, N209D, N209G, N209K, N209L, N209Q, N209R, N209V, L210A, L210C, L210F, L210G, L210H, L210I, L210Q, L210R, L210S, L210T, L210V, T211A, T211D, T211E, T211F, T211K, T211N, T211Q, T211R, T211S, T212A, T212C, T212E, T212F, T212G, T212H, T212K, T212L, T212M, T212S, T212W, K213A, K213C, K213D, K213F, K213I, K213L, K213M, K213N, K213Q, K213R, K213S, K213T, K213V, K213Y, V214A, V214C, V214T, V214W, A215D, A215E, A215F, A215I, A215K, A215L, A215Q, A215R, A215S, A215V, N216D, N216T, N216V, D217F, D217G, D217L, D217M, D217T, D217V, T218D, T218G, T218H, K219A, K219C, K219F, K219H, K219M, A220C, A220G, A220I, A220L, A220M, A220T, A220V, A220W, A221C, A221D, A221E, A221L, A221N, A221R, A221V, A221Y, A222D, A222I, A222L, A222P, A222R, A222W, A222Y, N223A, N223E, N223F, N223G, N223K, N223L, N223M, N223R, N223S, N223T, N223V, N223W, I224G, I224Q, T225A, T225G, T225L, L226C, L226M, L226Q, K227T, Q228N, Q228R, T229A, T229C, T229D, T229G, T229H, T229M, T229N, T229Q, T229R, T229V, V230F, V230L, V230M, V230Q, V230R, V230S, F231A, F231E, F231G, F231I, F231K, F231L, F231Q, F231S, F231V, F231W, F231Y, P232G, P232H, P232L, P232M, P232R, P232S, P232T, P232V, P232W, P232Y, K233A, K233C, K233E, K233F, K233G, K233L, K233P, K233R, K233S, K233V, K233W, K233Y, G234A, G234C, G234D, G234E, G234K, G234L, G234Q, G234R, G234V, G234W, G234Y, G235C, G235F, G235H, G235I, G235K, G235M, G235Q, G235R, G235T, G235W, G235Y, K236A, K236D, K236E, K236G, K236L, K236M, K236P, K236R, K236S, K236T, K236W, K236Y, T237D, T237F, T237I, T237K, T237M, T237Q, T237R, T237S, T237V, T237Y, D238A, D238E, D238F, D238G, D238H, D238I, D238K, D238L, D238M, D238N, D238P, D238Q, D238R, A239C, A239E, A239G, A239I, A239K, A239T, A240C, A240E, A240L, A240P, A240Q, A240T, A240V, A240W, A240Y, I241T, G242K, G242L, G242M, G242P, G242T, G242Y, T243I, T243M, T243R, T243V, V244A, V244E, V244G, V244L, V244R, T245E, T245G, T245L, T245M, T245N, T245Q, T245R, T245S, T246D, T246E, T246G, T246K, T246V, A247D, A247E, A247K, A247N, A247P, A247Q, A247R, A247S, A247V, A247W, S248A, S248E, S248F, S248H, S248I, S248L, S248Q, S248T, S248Y, K249A, K249D, K249G, K249H, K249I, K249L, K249M, K249N, K249P, K249Q, K249S, K249T, K249V, K249Y, S250H, S250M, S250W, I251F, I251L, I251V, I251W, I251Y, A252C, A252E, A252F, A252H, A252I, A252P, A252R, A252S, A252W, A252Y, G254D, G254F, G254I, G254L, G254M, G254Q, G254R, G254W, A255C, A255F, A255K, A255L, A255M, A255S, A255T, A255W, A255Y, S256A, S256C, S256F, S256G, S256K, S256L, S256M, S256N, S256Q, S256R, S256V, S256W, S256Y, A257D, A257G, A257I, A257N, A257T, A257V, D258A, D258L, D258M, D258W, V259E, V259L, V259S, V259T, T260A, T260D, T260G, T260I, T260K, T260V, S261A, S261D, S261H, S261R, S261W, S261Y, T262D, T262E, T262F, T262G, T262H, T262L, T262P, T262W, I263A, I263C, I263G, I263L, I263S, I263V, T264F, T264G, T264K, T264L, T264M, T264P, T264Q, T264R, T264S, T264Y, A265G, A265I, A265K, A265R, A265S, A265V, A266D, A266E, A266G, A266K, A266L, A266M, A266P, A266Q, A266S, A266T, S267A, S267D, S267K, S267M, S267N, S267P, S267Q, S267R, S267V, P268F, P268G, P268M, P268R, P268V, P268W, P268Y, K269G, K269R, K269V, K269Y, L270D, L270M, L270N, L270R, L270V, W271T, S272E, S272G, S272K, S272L, S272N, S272T, S272W, I273K, I273L, I273P, I273R, I273S, I273W, K274D, K274P, K274Q, K274R, N275K, N275M, N275V, N275W, N277F, N277K, N277R, L278A, L278G, L278H, L278I, L278K, L278M, L278P, L278Q, L278R, L278S, L278V, Y279M, Y279T, Y279W, T280A, T280D, T280E, T280F, T280H, T280M, T280Q, V281A, V281I, V281L, V281Q, R282E, R282F, R282H, R282I, R282K, R282N, R282S, R282T, R282V, R282W, T283M, T283R, T283V, E284A, E284D, E284F, E284H, E284L, E284M, E284N, E284Q, E284R, E284Y, V285H, V285I, V285T, L286A, L286C, L286D, L286F, L286N, L286R, L286T, L286W, L286Y, N287I, N287L, G288F, G288L, G288S, V291C, V291D, V291F, V291G, V291H, V291L, V291P, V291S, V291T, V291Y, L292A, L292D, L292E, L292H, L292Q, L292S, L292V, D293C, D293E, D293F, D293I, D293S, D293V, D293W, T294C, T294G, T294M, T294Q, T294S, Y295F, Y295G, Y295I, Y295L, Y295R, Y295S, Y295W, D296F, D296H, D296K, D296L, D296N, D296R, D296V, T297G, T297I, T297Q, T297R, T297S, E298G, E298I, E298L, E298M, E298R, E298T, Y299F, Y299S, F301C, F301L, R302E, R302I, R302K, R302M, R302N, R302T, W303A, W303C, W303D, W303F, W303S, W303T, T304D, T304E, T304I, T304K, T304P, T304S, T304W, G305E, G305F, G305L, G305M, G305N, G305P, G305T, G305W, F306D, F306K, F306L, F306S, D307A, D307E, D307F, D307G, D307L, D307M, D307N, D307Q, D307S, D307V, D307W, A308C, A308E, A308G, A308I, A308M, A308Y, T309C, T309D, T309E, T309I, T309K, T309M, T309S, T309V, S310A, S310C, S310G, S310H, S310I, S310L, S310M, S310N, S310R, S310V, G311E, G311F, G311L, G311Q, G311R, G311V, F312H, F312L, F312R, F312V, L314A, L314C, L314T, L314V, L314Y, N315S, G316A, G316C, G316N, G316R, G316S, G316W, G316Y, E317D, E317H, E317I, E317K, E317V, E317W, E317Y, K318C, K318E, K318F, K318G, K318H, K318L, K318M, K318N, K318P, K318Q, K318V, K320C, K320E, K320F, K320I, K320L, K320R, K320T, K320V, K320W, L321C, L321G, L321H, L321M, L321V, L321Y, K322C, K322F, K322I, K322N, K322P, K322Q, K322R, K322S, V324E, V324G, V324T, S325D, S325G, S325T, M326E, M326G, M326S, M326T, M326V, M326Y, H328C, H328D, H328F, H328G, H3281, H328L, H328M, H328R, H328T, D329S, D329T, G331V, A335C, A335G, A335L, A337D, A337E, A337G, A337L, A337Q, N338P, N338R, N338V, R340A, R340C, R340L, A341M, A341S, I342A, I342G, I342K, I342P, E343A, E343H, E343N, E343R, E343S, E343T, E343Y, R344G, R344L, R344Y, Q345F, Q345G, Q345K, Q345N, Q345S, V346A, V346C, V346F, V346I, V346L, V346S, E347A, E347C, E347D, E347F, E347I, E347R, E347S, I348A, I348D, I348G, I348M, I348Q, I348R, L349A, L349E, L349Q, L349S, L349V, Q350G, Q350N, Q350W, K351N, K351R, K351V, K351W, M352G, M352L, M352T, M352W, G353K, V354E, V354G, V354S, V354W, N355H, N355M, N355R, N355W, I357T, T359E, T359L, T360V, N362G, N362S, N362T, P363A, P363D, P363I, P363L, P363M, P363S, P363V, A364C, A364E, A364F, A364G, A364I, A364M, A364P, A364V, A364W, A365C, A365E, A365I, A365P, A365V, A365W, K366A, K366D, K366E, K366I, K366L, K366M, K366P, K366S, K366V, A367C, A367I, A367N, A367Q, L368A, L368E, L368Q, L368S, L368V, L368Y, I369A, I369D, I369E, I369G, I369K, I369M, I369R, I369V, I369W, D370C, D370L, D370Q, D370R, D370S, D370T, V371D, V371F, V371G, V371I, V371L, V371Q, V371S, C372P, N373G, N373L, N373R, E374K, E374L, E374R, K375D, K375I, K375N, K375Q, K375S, G376A, G376S, V377A, V377M, V377T, L378I, L378P, L378W, L378Y, V379A, V379C, V379M, V379N, V380M, V380P, V380S, E381A, E381C, E381G, E381L, E381Q, E381T, V383A, V383K, V383L, M386G, M386N, M386Q, M386S, M386V, W387H, W387L, N388A, N388E, N388L, N388R, R389A, R389C, R389E, R389K, R389M, R389N, R389Q, R389S, R389T, R389V, S390C, S390D, S390G, S390H, S390N, S390P, S390Q, S390T, S390V, K391E, N392D, G393A, G393E, G393N, G393R, G393S, G393V, N394L, T395A, T395C, T395F, T395H, T395I, T395M, T395N, T395Q, T395S, T395W, E396K, E396L, E396M, E396V, E396W, Y398M, Y398N, G399S, K400A, K400C, K400D, K400E, K400M, K400N, K400P, K400Q, K400S, K400T, K400V, W401F, W401H, W401K, W401L, W401M, W401R, F402T, F402W, F402Y, G403A, G403D, G403H, G403K, G403P, G403Q, G403S, G403T, G403V, G403Y, Q404F, Q404H, Q404L, Q404M, Q404P, Q404R, Q404S, Q404V, A405C, A405E, A405H, A405K, A405P, A405R, A405T, A405V, I406C, I406D, I406N, A407C, A407G, A407M, A407Q, A407S, A407T, A407W, G408D, G408I, G408M, G408N, G408W, D409N, D409W, N410C, N410R, N410Y, A411E, A411N, A411R, A411S, A411V, V412M, V412S, L413D, L413E, L413F, L413I, L413P, L413T, G414A, G414C, G414M, G414N, G414R, G414T, G414W, G415A, G415Q, G415R, D416I, D416M, D416R, D416T, D416Y, K417C, K417F, K417G, K417R, K417T, D418L, D418P, D418R, D418Y, E419M, E419R, E419W, T420E, T420F, T420G, T420K, T420R, T420V, W421L, W421Q, W421S, A422P, A422T, A422V, K423D, K423L, K423M, K423R, F424C, F424L, F424N, F424T, D425E, L426C, L426M, L426Q, T427D, T427F, T427G, T427K, T427M, T427P, T427Q, T427R, T427S, T427W, S428F, S428K, S428W, T429D, T429P, I430C, I430D, I430E, I430L, I430M, I430Q, I430S, I430T, I430W, N431D, N431E, N431G, N431L, N431M, N431R, N431V, N431Y, R432A, R432E, R432F, R432G, R432N, R432Q, R432V, R432Y, D433C, D433G, D433H, D433I, D433P, D433Q, D433W, R434L, R434M, R434N, R434P, R434S, R434T, R434V, N435E, N435F, N435H, N435K, N435L, N435M, N435R, N435V, N435W, A436C, A436D, A436E, A436G, A436I, A436L, A436M, A436Q, A436S, P437A, P437D, P437K, P437L, P437Q, P437R, P437S, P437V, P437W, S438G, V439C, V439E, V439G, V439I, V439K, V439Q, V439T, V439Y, I440C, I440D, I440F, I440K, I440P, I440R, I440S, I440T, I440V, I440W, M441A, M441E, M441G, M441Q, M441R, M441T, M441V, W442E, W442G, W442M, W442P, W442Q, W442R, S443C, S443D, S443G, S443M, S443Q, S443Y, L444C, L444D, L444E, L444F, L444G, L444H, L444K, L444Q, L444V, L444W, G445A, G445C, G445V, N446D, N446T, M448A, M448C, M448D, M448E, M448I, M448L, M448P, M448Q, M448S, M448V, M448W, M449D, M449E, M449F, M449T, M449V, M449W, M449Y, E450F, E450S, E450T, E450V, E450W, G451C, G451F, G451L, G451P, G451V, G451W, I452G, I452K, I452L, I452M, I452Q, I452S, I452V, S453C, S453F, S453G, S453H, S453L, S453M, S453N, S453P, S453Q, S453R, S453V, G454L, G454W, S455A, S455E, S455K, S455M, S455P, S455R, S455V, S455W, V456A, V456D, V456E, V456F, V456K, V456L, V456W, S457E, S457H, S457K, S457L, S457M, S457P, S457Q, S457T, S457V, G458A, G458C, G458D, G458F, G458L, G458P, G458Q, G458S, G458V, G458W, F459A, F459C, F459E, F459G, F459N, F459R, F459S, F459T, F459W, P460C, P460M, P460Q, P460W, P460Y, A461D, A461G, A461M, A461N, A461Q, A461S, A461V, A461Y, T462C, T462E, T462F, T462L, T462M, T462S, S463G, S463K, S463Q, S463R, S463T, S463V, A464E, A464H, A464L, A464M, A464P, A464V, A464W, K465C, K465F, K465G, K465L, K465Q, K465R, K465V, K465W, K465Y, L466A, L466C, L466D, L466E, L466F, L466G, L466M, L466P, L466Q, L466S, L466V, L466Y, V467A, V467C, V467D, V467E, V467G, V467T, V467W, A468D, A468E, A468F, A468K, A468L, A468P, A468S, A468V, A468W, W469A, W469C, W469D, W469G, W469L, W469M, W469R, W469V, W469Y, T470E, T470L, T470M, T470Q, K471F, K471G, K471Q, K471W, K471Y, A472G, A472Y, A473E, A473M, A473P, D474A, D474C, D474E, D474K, D474M, D474R, D474W, S475E, S475F, S475Q, S475T, S475V, T476C, T476L, T476S, R477A, R477C, R477G, R477L, P478A, P478D, P478G, P478L, P478V, M479G, M479I, M479R, M479W, T480C, T480G, T480Q, K485E, K485R, K487A, K487C, K487F, K487G, K487N, K487S, K487W, A488C, A488G, A488H, A488L, A488N, A488S, A488V, N491A, N491E, N491W, E492A, E492W, S493E, S493G, S493H, S493L, S493M, S493Q, S493V, N494A, N494I, N494M, N494R, N494V, T495K, T495R, T495V, T495W, M496A, M496F, M496T, G497D, D498A, D498C, D498M, D498S, N499K, N499R, N499T, N499Y, L500A, L500E, L500N, L500V, T501C, T501G, T501M, A502L, A502Q, N503A, N503E, N503M, N503S, G504H, G504K, G504P, G505A, G505D, G505E, G505H, G505L, G505N, G505R, G505S, G505V, V506C, V506D, V506E, V506G, V506I, V506L, V506P, V506R, V506S, V506T, V506W, V507A, V507F, V507G, V507L, V507N, V507P, V507R, V507S, V507T, G508C, G508E, T509A, T509D, T509E, T509I, T509K, T509M, T509Q, T509S, T509V, T509Y, N510A, N510F, N510I, N510Q, Y511A, Y511K, S512C, S512E, S512F, S512G, S512I, S512M, S512Q, S512T, S512V, S512Y, D513C, D513G, D513K, D513L, D513M, D513P, D513Q, D513R, D513W, G514F, G514L, G514N, G514P, G514R, A515C, A515D, A515E, A515F, A515G, A515K, A515P, A515R, A515S, N516C, N516E, N516G, N516I, N516M, N516Q, N516S, N516T, N516V, Y517G, Y517I, Y517N, D518Q, D518Y, K519C, K519E, K519F, K519G, K519I, K519L, K519M, K519N, K519Q, K519S, K519T, I520A, I520C, I520G, I520H, I520M, I520N, I520Q, I520S, I520T, I520W, I520Y, R521A, R521C, R521K, R521N, R521Q, R521V, T522G, T522I, T522N, T523A, T523M, H524R, H524V, P525D, P525G, P525R, P525T, P525V, S526G, S526I, S526W, W527A, W527E, W527G, W527H, W527N, W527R, W527S, W527V, A528C, A528E, A528G, A528I, A528L, I529F, I529G, I529L, I529Y, Y530A, Y530M, G531E, G531S, G531T, T534A, T534I, T534Q, A535E, A535G, A535I, A535M, A537D, A537M, A537P, A537S, I538H, I538M, N539G, N539W, S540E, S540G, S540M, G542E, G542Q, G542S, G542T, I543V, I543W, N545G, N545I, N545Q, N545S, R546A, R546C, R546L, R546P, R546S, T547A, T547D, T547H, T547K, T547N, T547S, T548D, T548E, T548F, T548K, T548L, T548P, T548W, G549D, G549F, G549P, G549W, G550Q, G550R, G550S, A551D, A551I, A551Q, S553C, S553F, S553N, S553P, S553R, S553T, S553V, S554F, S554N, S554R, S554T, S554V, S554W, D555E, D555P, D555S, K556A, K556C, K556R, K556W, Q557F, Q557R, Q557S, L558E, L558H, L558I, L558P, T559A, T559G, T559I, T559Q, T559V, T559Y, S560P, S560V, Y561R, N563R, N563S, S564A, S564C, S564F, S564W, A565C, A565D, A565E, A565F, A565G, A565I, A565L, A565M, A565R, A565S, A565T, A565V, A565W, G567N, G567Q, G567V, A570G, A570K, A570L, A570M, A570S, A570T, A570W, V571W, A572S, A572W, S573G, S573K, S573Y, S574K, S574Q, S574V, S574W, A575D, A575M, A575V, W576A, W576F, W576V, W576Y, Y577G, Y577I, Y577L, Y577R, D578E, D578M, D578N, D578T, V579E, V579G, V579L, V579T, V580A, V580D, V580E, V580K, V580L, V580S, Q581F, Q581G, Q581P, Q581R, Q581S, Q581T, Q581Y, R582A, R582D, R582G, R582I, R582L, R582Y, D583V, D583W, F584E, F584I, F584W, V585I, V585M, V585Q, A586C, A586D, A586H, A586K, G587A, G587C, T588C, T588D, T588G, T588I, T588L, T588M, T588P, Y589A, Y589I, Y589Q, Y589V, Y589W, V590A, V590H, V590I, W591F, T592C, T592L, T592Q, T592S, G593C, G593I, F594C, F594I, F594L, F594M, D595E, D595Q, D595S, L597C, L597D, L597E, L597T, G598N, P600A, P600E, P600G, P600S, N604E, N604S, G605R, T606S, G607A, G607Q, G607S, S608E, S608I, S608M, S608N, S608Q, S608T, S608V, G609A, G609H, G609L, G609N, G609R, G609S, A610D, A610F, A610M, A610T, V611K, G612N, G612T, G612V, S613A, W614A, W614F, W614P, P615L, P615S, P615V, S616A, S616W, N619A, N619I, N619L, N619S, S620G, Y621W, I624A, I624V, V625A, V625E, V625F, V625M, V625Y, T627F, T627G, T627K, T627Q, A628C, A628D, A628N, G629T, F630A, F630C, F630D, F630G, F630S, F630Y, P631A, P631D, P631G, P631H, P631S, P631V, P631Y, K632C, K632D, K632G, K632T, D633G, T634A, T634E, T634F, T634S, T634V, Y635R, Y636K, F637C, F637G, F637I, F637S, F637T, F637V, Y638A, Y638W, Q639D, Q639N, Q639R, S640C, S640D, Q641T, W642I, N643R, D644C, D644G, D644Y, D645S, D645V, V646C, V646L, V646N, V646R, V646S, H647G, H647V, T648C, L649V, H650E, H650F, H650R, I651F, I651T, I651V, L652C, L652D, L652V, L652W, P653Q, A654D, A654K, A654M, A654R, W655F, W655R, N656A, N656K, N656V, E657K, E657R, E657V, V659D, V659N, A661E, A661G, A661H, A661K, A661L, A661M, A661Q, A661W, K662H, K662S, K662V, K662W, K662Y, N667L, N667R, P669A, P669E, P669F, P669L, P669R, P669T, P669W, V670C, V672L, Y673E, Y673G, Y673I, Y673R, Y673S, Y673W, T674C, T674D, T674G, T674H, T674M, T674Q, D675A, D675E, D675P, D675Q, D675S, D675V, D675Y, A676C, A676E, A676G, A676K, A676L, A676P, A676S, A676W, A677E, A677G, A677L, A677R, A677T, A677V, A677Y, K678A, K678C, K678T, K678V, V679G, V679Q, V679S, V679T, V679Y, K680A, K680E, K680G, K680H, K680I, K680L, K680N, K680Q, K680S, K680V, K680W, L681E, L681F, L681G, L681M, L681S, L681T, Y682D, Y682E, Y682I, Y682M, Y682S, Y682V, Y682W, F683H, F683L, F683M, F683Q, F683R, F683W, T684A, T684D, T684G, T684L, T684R, T684S, T684V, P685A, P685E, P685I, P685L, P685W, K686A, K686E, K686I, K686M, K686T, K686V, G687A, G687K, G687N, G687P, G687Q, G687R, S688C, S688D, S688E, S688G, S688K, S688L, S688P, S688T, T689A, T689D, T689E, T689G, T689L, T689P, T689S, T689W, T689Y, E690D, E690M, E690P, E690T, E690V, K691E, K691H, K691N, K691P, K691R, K691S, R692G, R692H, R692I, R692L, R692P, R692S, R692T, R692V, R692W, L693A, L693M, L693P, I694L, I694W, G695C, G695K, G695L, G695R, G695W, E696A, E696L, E696R, K697A, K697E, K697G, K697R, K697V, K697W, S698C, S698D, S698E, S698I, S698L, S698M, S698P, S698Q, S698R, S698T, T700A, T700C, T700D, T700E, T700G, T700K, T700Y, K701A, K701D, K701E, K701G, K701H, K701L, K701M, K701P, K701S, K701W, T703E, T703I, T703K, T703W, T704M, T704R, T704Y, A705C, A705D, A705E, A705K, A705N, A705P, A705R, A705V, A705W, A706C, A706G, A706T, A706W, A706Y, Y708C, Y708F, Y708G, Y708K, Y708L, Y708P, Y708T, T709M, Y710C, Y710D, Y710E, Y710G, Y710M, Y710N, Y710T, Y710V, Y710W, Q711A, Q711D, Q711G, Q711L, Q711M, Q711T, Q711Y, V712G, V712M, V712P, V712Q, V712R, Y713A, Y713E, Y713G, Y713L, Y713Q, E714D, E714H, E714I, E714K, E714M, E714N, E714R, E714S, E714V, E714Y, G715K, A716C, A716G, A716L, A716M, A716P, A716R, A716T, A716V, D717C, D717G, D717L, D717S, K718A, K718L, K718Q, K718T, K718W, K718Y, D719E, D719L, D719P, D719S, D719T, D719V, S720A, S720M, S720R, S720Y, T721D, T721H, T721K, T721L, T721N, T721P, T721Q, T721S, T721V, T721W, T721Y, A722Q, A722T, A722V, H723G, H723P, K724A, K724G, K724R, N725A, N725D, N725I, M726A, M726D, M726E, M726K, M726Q, M726V, M726W, Y727L, Y727T, L728C, L728K, L728Q, L728S, L728T, L728W, T729A, T729E, T729M, W730A, N731A, N731E, N731Q, N731S, N731Y, V732G, V732P, V732R, V732W, P733A, P733R, P733W, W734D, W734G, W734R, W734V, A735Q, A735R, A735S, A735T, A735W, E736S, G737F, G737K, G737N, G737Y, T738G, T738L, T738S, I739E, I739K, I739M, I739W, I739Y, S740D, S740E, S740F, S740H, S740L, A741I, A741P, A741S, A741V, E742D, E742M, E742Q, E742V, A743C, A743E, A743H, A743I, A743L, Y744A, Y744E, Y744I, Y744K, Y744L, Y744R, D745C, D745F, D745N, D745R, E746A, E746C, E746K, E746T, N747E, N747F, N747G, N747P, N747R, N748S, R749H, R749M, R749S, R749T, L750G, L750M, L750P, L750Q, L750S, I751C, I751H, I751Q, I751S, P752A, P752C, P752H, P752L, P752S, P752V, P752Y, E753Q, G754H, G754I, G754P, G754R, S755C, S755I, S755T, T756E, T756N, T756P, T756Q, T756S, T756W, E757A, G758A, G758V, N759D, N759R, N759S, N759V, A760G, A760N, A760P, A760Q, S761A, S761K, S761Q, V762D, V762G, V762K, V762W, T765A, T765P, T765R, T765W, G766M, G766S, A768H, A769F, A769G, A769I, A769N, A769V, A769Y, K770H, L771A, L771I, K772A, K772C, K772P, K772V, K772W, A773C, A773G, A773H, A773M, A773R, A773S, A773V, D774A, D774R, A775L, A775V, A775W, D776I, D776L, D776R, D776S, D776V, R777D, R777E, R777G, R777H, R777P, R777S, R777T, K778A, K778C, K778F, K778G, K778L, K778N, K778R, T779C, T779I, I780V, T781A, T781C, T781E, T781F, T781G, T781M, T781P, T781R, T781Y, A782C, A782E, A782K, A782N, A782P, A782Q, A782Y, D783A, D783C, D783E, D783R, G784A, G784F, G784L, G784S, G784T, K785C, K785I, K785S, K785V, K785W, K785Y, D786S, D786V, L787D, L787K, L787P, L787T, L787Y, S788A, S788G, S788I, Y789C, Y789D, Y789I, Y789V, I790A, I790C, I790F, I790R, I790V, E791A, E791D, E791F, E791M, E791S, E791T, E791V, E791W, V792C, V792G, V792L, V792S, V792Y, D793C, D793F, D793H, D793K, D793N, D793Y, V794C, V794D, V794L, V794Q, V794T, V794W, T795P, T795Y, D796M, D796Q, D796S, D796T, A797H, A797K, N798G, N798I, N798P, N798Q, G799D, G799K, G799L, G799M, G799Q, G799Y, H800A, H800F, H800G, H800L, H800S, H800V, I801C, I801E, I801W, V802E, V802I, V802P, V802S, V802Y, P803A, P803F, P803G, P803K, P803S, P803Y, D804E, D804G, D804K, D804N, D804S, A805C, A805F, A805G, A805I, A805N, A805P, A806F, A806I, A806Q, N807F, N807Q, N807V, N807W, R808C, R808F, R808G, R808I, R808N, R808P, R808Q, V809A, V809C, V809L, V809M, V809P, T810L, T810P, T810Q, T810R, T810Y, F811L, F811Y, D812E, D812F, D812I, D812Q, V813F, V813H, V813T, V813W, V813Y, K814G, K814H, K814I, K814L, K814P, G815A, G815F, G815M, G815P, G815V, A816C, A816D, A816F, A816I, A816N, A816V, A816W, G817C, G817H, G817I, G817N, G817S, K818D, K818F, K818L, K818Q, K818R, K818S, K818V, K818W, K818Y, L819F, L819W, V820C, V820F, V820I, V820K, V820R, V820W, G821A, G821C, G821E, G821F, G821I, G821K, G821M, G821N, G821V, G821Y, V822A, V822D, V822E, V822T, D823E, N824A, N824C, N824G, N824Q, G825A, S826A, S826F, S826G, S826I, S826L, S826R, S826W, S827C, S827Q, P828C, P828G, P828I, P828L, P828Y, D829C, D829I, D829S, D829T, D829V, H830E, H830G, H830M, H830P, H830Q, H830R, H830V, D831A, D831F, D831G, D831I, D831M, D831P, D831R, D831V, S832E, S832F, S832G, S832L, S832M, S832P, S832R, S832V, S832W, Y833C, Y833D, Y833E, Y833I, Y833K, Y833N, Y833P, Y833V, Q834F, Q834G, Q834M, A835D, A835E, A835F, A835H, A835K, A835W, D836C, D836E, D836H, D836Q, D836R, D836S, D836T, D836V, D836W, D836Y, N837D, N837F, N837G, N837H, N837L, N837P, N837T, R838D, R838F, R838G, R838K, R838M, R838N, R838S, R838W, K839A, K839C, K839D, K839E, K839G, K839L, K839N, K839P, K839R, A840G, A840I, A840P, A840V, F841C, F841D, F841I, F841K, F841W, S842A, S842L, S842M, G843A, G843C, K844A, K844G, K844L, K844W, K844Y, V845N, V845W, L846G, L846I, L846M, L846S, A847L, A847T, I848M, V849A, V849L, V849S, V849T, Q850C, Q850G, Q850I, Q850L, Q850T, Q850V, Q850Y, S851C, S851D, S851E, S851L, S851T, T852D, T852G, T852L, K853N, K853P, K853Q, K853V, E854C, E854I, E854M, E854R, A855K, A855V, A855Y, E857P, E857V, I858D, I858E, I858F, I858G, I858K, I858M, I858P, I858Q, I858Y, T859V, V860T, V860Y, T861F, T861I, T861Q, T861V, T861W, A862C, A862P, A862V, K863F, K863I, K863L, K863N, K863W, A864E, A864H, A864K, A864L, D865A, D865G, G866H, G866R, L867W, S870E, S870M, S870R, S870T, T871P, V872C, V872G, K873G, K873Y, I874G, A875R, T877A, V879P, V879S, P880S, G881W, T882A, T882M, T882R, S883L, T884A, E885V, K886E, K886L, K886V, K886W, T887A, T887D, T887F, T887G, T887N, T887R, T887V, V888A, V888D, V888G, R889G, Y892D, Y892P, Y892R, Y893E, Y893G, S894D, S894G, R895M, N896M, Y897V, Y898T, V899G, K900E, K900G, T901G, T901Q, T901R, T901V, T901Y, G902A, G902D, G902F, G902L, G902P, G902Q, G902R, G902S, G902W, N903D, N903I, K904D, K904E, K904M, K904N, K904R, K904S, K904V, K904W, P905A, P905C, P905R, P905V, P905W, P905Y, I906A, I906D, I906N, I906S, I906T, I906W, I906Y, L907F, L907S, L907Y, P908C, P908D, P908G, P908I, P908L, P908M, P908T, S909E, S909F, S909G, S909W, S909Y, D910C, D910I, D910S, D910W, V911A, V911S, E912A, E912K, E912L, E912T, E912V, V913G, V913Q, V913R, V913W, R914A, R914E, R914F, R914I, R914K, R914V, R914W, R914Y, Y915A, Y915C, Y915G, Y915I, Y915M, Y915Q, Y915V, S916G, D917C, D917E, D917F, D917L, D917M, D917Q, D917S, G918E, G918H, G918T, G918V, G918W, T919D, T919K, T919Q, T919W, T919Y, S920C, S920E, S920M, S920P, S920R, S920V, S920W, D921C, D921P, D921Q, D921V, R922A, R922G, R922M, R922V, R922W, Q923A, Q923C, Q923E, Q923L, Q923M, Q923V, Q923W, N924A, N924L, N924P, N924Q, N924S, N924W, V925A, V925C, V925E, V925G, V925K, V925N, V925S, V925W, T926G, T926P, T926R, T926S, T926V, T926W, W927C, W927G, W927P, D928A, D928E, D928H, D928L, D928Q, A929C, A929P, A929V, V930A, V930E, V930I, V930K, V930M, V930T, S931G, S931P, S931R, D932F, D932R, D932S, D932T, D932V, D933I, D933R, D933S, Q934S, Q934V, I935A, I935C, I935D, I935E, I935L, I935P, I935V, I935W, A936I, A936L, A936Q, A936R, A936Y, K937G, K937I, K937M, K937P, K937Q, K937R, K937V, A938C, A938H, A938N, A938T, A938V, A938W, G939D, G939K, S940C, S940E, S940M, S940R, S940T, S940V, S940W, F941C, F941M, F941W, S942A, S942E, S942K, S942L, S942P, S942T, S942V, V943A, V943G, V943H, V943Q, V943R, A944D, A944G, A944H, A944P, A944R, A944V, G945E, G945P, G945T, T946A, T946E, T946G, T946L, T946P, T946V, T946W, V947G, V947H, V947L, V947M, V947P, V947R, V947T, A948C, A948I, A948R, A948W, G949A, G949F, G949V, Q950D, Q950G, Q950K, Q950M, Q950W, K951D, K951G, K951P, K951Q, K951S, K951W, K951Y, I952H, I952Q, S953F, S953M, S953N, S953R, S953W, V954D, V954L, V954Q, V954S, V954T, R955A, R955C, R955E, R955K, R955Q, R955W, V956A, V956D, V956G, V956H, V956I, V956M, V956Q, V956W, T957D, T957S, T957W, M958D, M958I, M958K, I959A, I959L, I959S, I959V, I959Y, D960G, D960H, D960L, D960P, D960S, D960W, E961D, E961F, E961K, E961P, E961S, E961T, I962A, I962C, I962D, I962G, I962K, I962N, I962Q, I962T, G963C, G963E, G963L, G963P, A964C, A964E, A964H, L965C, L965E, L965G, L965K, L965M, L965P, L965Q, L965S, L965V, L965Y, L966A, L966G, L966H, L966K, L966N, L966P, L966Q, L966S, L966T, L966V, N967C, N967D, N967I, N967L, N967M, N967P, N967S, N967T, N967V, Y968G, Y968L, Y968Q, Y968V, S969C, S969D, S969G, S969H, S969I, S969L, S969M, S969P, S969Q, S969Y, A970I, A970L, S971E, S971F, S971G, S971H, S971V, S971W, P973C, P973D, P973K, P973N, P973Q, P973R, P973V, P973W, P973Y, V974C, V974E, V974G, V974N, V974T, V974Y, G975D, G975F, G975K, G975L, G975Q, G975V, T976D, T976F, T976G, T976K, T976L, T976P, T976S, T976Y, P977A, P977C, P977K, P977R, P977T, P977Y, A978F, A978G, A978M, A978N, A978P, A978R, A978S, A978Y, V979G, V979N, V979R, V979Y, L980A, L980F, L980H, L980I, L980K, L980N, L980Q, L980T, L980Y, P981L, P981M, G982A, G982H, G982M, G982P, G982Q, G982W, R984P, R984S, P985E, P985F, P985H, P985K, P985L, P985W, A986C, A986E, A986F, A986I, A986K, A986L, A986M, A986N, A986S, A986W, V987A, V987C, V987F, V987I, V987K, V987L, V987Q, V987T, L988A, L988C, L988E, L988G, L988H, L988M, L988Q, L988R, L988S, L988V, L988Y, P989A, P989C, P989D, P989G, P989H, P989I, P989M, P989N, P989Q, P989W, D990F, D990P, D990S, D990W, G991C, G991F, G991H, G991K, G991P, G991Y, T992E, T992H, T992M, T992N, T992Y, V993D, V993G, V993N, V993S, T994I, T994S, T994V, S995E, S995L, S995R, S995V, A996Q, A996R, A996V, N997A, N997C, N997E, N997K, N997L, N997S, N997V, N997W, N997Y, F998M, F998W, A999F, A999G, A999L, A999M, A999R, A999S, V1000C, V1000L, V1000M, V1000N, V1000P, V1000W, D1001G, D1001K, D1001L, D1001M, D1001Q, D1001S, D1001T, D1001V, D1001Y, W1002A, W1002D, W1002E, W1002H, W1002N, W1002P, W1002Q, W1002S, T1003F, T1003G, T1003L, T1003N, T1003P, T1003R, T1003S, T1003W, T1003Y, K1004D, K1004E, K1004F, K1004G, K1004H, K1004M, K1004P, K1004R, K1004S, K1004V, P1005I, P1005N, P1005Q, P1005V, P1005Y, A1006C, A1006I, A1006N, A1006P, A1006S, A1006V, A1006W, A1006Y, D1007C, D1007L, D1007P, D1007V, T1008G, V1009G, V1009S, Y1010A, Y1010P, Y1010R, Y1010T, N1011A, N1011S, N1011T, N1011W, T1012E, T1012H, T1012I, T1012Q, T1012Y, A1013D, A1013K, A1013Q, A1013T, A1013V, G1014E, G1014I, G1014L, G1014M, G1014V, G1014W, G1014Y, T1015A, T1015F, T1015G, T1015V, V1016C, V1016D, V1016P, K1017E, K1017G, V1018I, V1018K, V1018L, V1018M, V1018R, V1018S, V1018W, T1021C, T1021E, T1021F, T1021G, T1021K, T1021L, T1021S, T1021V, A1022H, A1022L, A1022S, A1022Y, T1023D, T1023M, T1023Q, T1023R, V1024E, V1024G, V1024H, V1024K, V1024N, V1024R, V1024S, V1024W, G1026E, G1026H, G1026L, G1026R, G1026S, G1026V, G1026Y, K1027C, K1027N, K1027Q, K1027R, K1027V, E1028G, E1028S, E1028T, F1029I, F1029K, F1029L, F1029P, F1029V, F1029W, F1029Y, K1030D, K1030F, K1030H, K1030L, K1030M, K1030W, V1031H, V1031K, V1031Y, A1033G, A1033S, A1033V, T1034G, T1034H, T1034N, T1034W, I1035D, I1035G, I1035Q, R1036G, R1036L, R1036T, R1036Y, V1037C, V1037F, V1037P, V1037Q, Q1038A, Q1038D, Q1038K, R1039S, R1039V, S1040A, S1040M, S1040N, S1040R, S1040W, Q1041P, V1042N, T1043F, T1043G, T1043N, T1043R, I1044A, I1044L, G1045S, S1046I, S1046M, S1047D, V1048C, V1048F, V1048G, V1048I, V1048M, V1048Q, G1050L, G1050S, G1050V, N1051A, N1051E, N1051K, A1052C, A1052K, A1052M, A1052P, A1052R, L1053A, L1053W, R1054C, R1054L, R1054N, L1055R, L1055T, Q1057A, Q1057E, Q1057P, Q1057R, N1058R, N1058S, N1058V, N1058W, I1059W, P1060G, P1060N, P1060Q, P1060S, P1060T, A1061E, A1061G, A1061K, A1061W, D1062A, D1062F, D1062G, D1062I, D1062L, D1062M, D1062P, D1062S, K1063D, K1063M, Q1064C, Q1064M, Q1064R, Q1064T, Q1064V, S1065A, S1065C, S1065E, S1065G, S1065T, S1065W, D1066A, D1066G, D1066M, D1066V, D1066W, T1067G, T1067M, L1068C, L1068E, L1068P, L1068Q, L1068Y, D1069G, D1069K, D1069R, D1069W, A1070P, A1070T, I1071M, I1071R, I1071W, K1072E, K1072G, K1072P, K1072Q, K1072S, D1073F, D1073L, D1073M, D1073P, D1073W, G1074I, G1074L, G1074R, S1075C, S1075G, S1075I, S1075L, S1075V, T1076C, T1076E, T1076H, T1076Q, T1076S, T1077K, T1077L, T1077R, V1078D, V1078E, V1078L, V1078W, D1079G, D1079L, N1081D, N1081E, N1081G, T1082A, T1082C, T1082E, T1082F, T1082G, T1082K, T1082N, T1082S, G1083E, G1083F, G1083L, G1083P, G1083S, G1084C, G1084M, G1084V, G1084W, G1084Y, G1085A, G1085P, G1085R, G1085S, A1086H, A1086K, A1086Q, A1086R, A1086T, N1087A, N1087E, N1087I, N1087R, N1087V, N1087W, P1088D, P1088E, P1088G, P1088R, P1088W, S1089C, S1089E, S1089G, S1089K, S1089Q, S1089R, S1089V, A1090F, A1090G, A1090I, A1090K, W1091A, W1091E, W1091G, W1091H, W1091T, W1091V, W1091Y, T1092A, T1092E, T1092G, T1092K, T1092Q, T1092S, T1092V, N1093A, N1093G, N1093L, N1093P, N1093Q, N1093T, N1093V, W1094D, W1094E, W1094P, W1094R, W1094T, A1095P, A1095R, A1095T, A1095W, Y1096A, Y1096D, Y1096H, Y1096L, Y1096R, S1097D, S1097E, S1097K, S1097L, S1097T, S1097W, K1098D, K1098F, K1098G, K1098Q, K1098S, A1099C, A1099D, A1099F, A1099S, A1099V, A1099W, G1100D, G1100E, G1100H, G1100M, G1100N, G1100T, H1101K, H1101L, H1101Q, H1101R, H1101V, N1102E, N1102F, N1102H, N1102K, N1102L, N1102Q, N1102R, N1102T, T1103A, T1103E, T1103H, T1103S, T1103W, A1104I, A1104K, A1104R, E1105L, E1105S, I1106T, I1106V, T1107C, T1107M, T1107R, T1107S, F1108D, F1108K, F1108L, F1108T, F1108W, E1109A, E1109D, E1109L, E1109W, A1111G, A1111S, E1113D, E1113G, E1113P, E1113V, Q1114E, Q1114I, Q1114L, Q1114R, Q1114S, Q1114T, Q1114V, Q1115A, Q1115K, Q1115L, Q1115P, Q1115T, Q1115W, L1116D, L1116G, L1116H, L1116K, L1116V, L1116W, G1117E, G1117I, G1117M, G1117R, G1117S, G1117T, G1117V, G1117W, Q1118A, Q1118M, Q1118S, Q1118T, Q1118W, I1119D, I1119E, I1119G, I1119N, I1119S, V1120N, V1120S, V1120T, M1121G, M1121K, M1121N, M1121P, M1121S, M1121V, M1121Y, Y1122A, Y1122C, Y1122I, Y1122K, Y1122R, Y1122V, Y1122W, F1123E, F1123H, F1123R, F1123T, F1124E, F1124R, F1124V, F1124W, R1125D, R1125E, R1125F, R1125K, R1125T, R1125V, R1125W, D1126H, D1126K, D1126L, D1126R, S1127F, S1127I, S1127K, S1127M, S1127Q, S1127T, S1127W, N1128A, N1128C, N1128R, N1128S, N1128T, N1128W, A1129E, A1129L, A1129N, A1129Q, A1129R, A1129V, V1130A, V1130G, V1130P, V1130R, V1130S, R1131A, R1131N, R1131Q, R1131S, R1131W, F1132E, F1132K, F1132M, F1132P, F1132Q, F1132T, P1133D, P1133G, P1133L, P1133Q, P1133R, P1133V, D1134E, D1134G, D1134L, A1135E, A1135K, A1135L, A1135M, A1135S, A1135W, A1135Y, G1136A, G1136E, G1136P, G1136Q, G1136T, K1137A, K1137C, K1137G, K1137L, K1137P, K1137Q, K1137R, K1137S, K1137T, K1137V, T1138R, T1138Y, K1139A, K1139L, K1139R, K1139T, I1140A, I1140C, I1140G, I1140L, I1140M, I1140P, I1140R, I1140T, Q1141A, Q1141C, Q1141G, Q1141K, Q1141N, Q1141P, Q1141T, Q1141W, I1142E, I1142R, I1142S, I1142W, I1142Y, S1143G, A1144C, A1144D, A1144E, A1144N, A1144P, A1144S, A1144T, A1144V, A1144W, D1145E, D1145R, D1145T, G1146A, G1146C, G1146D, G1146K, G1146L, G1146R, G1146V, K1147A, K1147G, K1147T, K1147V, N1148H, N1148I, N1148K, N1148P, N1148Q, N1148R, N1148S, N1148T, N1148W, W1149C, W1149G, W1149I, W1149K, W1149N, W1149Q, W1149S, W1149T, W1149V, W1149Y, T1150G, T1150K, T1150P, D1151C, D1151G, D1151R, D1151T, D1151W, L1152A, L1152C, L1152E, L1152Q, L1152W, A1153E, A1153G, A1153K, A1153L, A1154C, A1154D, A1154E, A1154G, A1154R, A1154S, T1155E, T1155L, T1155Q, T1155R, T1157V, T1157W, I1158R, I1158S, I1158W, A1159C, A1159E, A1159I, A1159P, A1159R, A1159V, A1160K, A1160L, A1160Q, A1160S, Q1161A, Q1161P, Q1161S, E1162A, E1162C, E1162D, E1162F, E1162I, E1162N, E1162Q, E1162T, E1162W, E1162Y, E1165D, E1165H, E1165L, E1165M, E1165R, E1165S, E1165W, R1166D, R1166K, R1166Q, V1167A, V1167C, V1167L, V1167P, V1167R, K1168L, K1168Q, K1168R, K1168W, P1169M, P1169R, P1169S, Y1170E, Y1170K, Y1170M, Y1170Q, Y1170R, Y1170V, T1171A, T1171G, T1171M, T1171Q, T1171R, T1171S, Y1172D, Y1172E, Y1172H, Y1172I, Y1172K, Y1172L, Y1172S, Y1172V, D1173A, D1173E, D1173F, D1173G, D1173K, D1173L, D1173P, D1173R, D1173T, D1173W, F1174D, F1174P, F1174Q, F1174R, F1174S, F1174T, F1174V, F1174W, A1175G, A1175I, A1175N, A1175Q, A1175S, A1175V, A1175Y, V1177N, V1177P, V1177S, V1177T, G1178M, G1178Q, G1178S, G1178T, A1179L, A1179P, A1179Q, A1179W, T1180A, T1180G, T1180I, T1180L, T1180M, T1180Q, T1180S, T1180Y, F1181E, F1181L, F1181V, V1182M, K1183A, K1183E, K1183T, K1183V, V1184C, V1184E, V1184K, V1184L, V1184P, V1184Q, V1184Y, T1185Q, T1185V, V1186S, N1188V, N1188W, A1189C, A1189K, A1189T, A1189V, D1190R, D1190S, D1190T, D1190Y, T1191E, T1191L, T1192H, T1192P, T1193G, P1194A, P1194E, P1194G, P1194W, S1195G, V1197A, V1198E, C1199D, C1199T, A1200G, A1200V, A1200W, L1202A, L1202C, T1203E, T1203K, E1204G, E1204S, I1205V, K1208R, K1208V, K1208W, T1209A, T1209C, T1209E, T1209K, T1209N, T1209Q, T1209R, T1209W, A1210D, A1210E, A1210G, A1210K, A1210L, A1210Q, A1210R, A1210T, A1210W, T1211C, T1211D, T1211E, T1211G, T1211H, T1211K, T1211P, T1211Q, T1211R, T1211S, T1211V, K1213A, K1213D, K1213S, K1213T, K1213W, F1214A, F1214E, F1214K, F1214L, F1214P, F1214R, F1214S, F1214V, V1215D, V1215E, V1215K, V1215L, V1215Q, V1215S, V1215W, T1216A, T1216L, T1216P, T1216Q, T1216R, N1217A, N1217D, N1217E, N1217F, N1217P, N1217R, N1217S, N1217T, T1218A, T1218C, T1218E, T1218G, T1218Q, T1218S, T1218V, T1218W, S1219A, S1219E, S1219F, S1219I, S1219K, S1219R, S1219V, A1220C, A1220G, A1220L, A1220P, A1220R, A1220V, A1221D, A1221G, A1221K, A1221L, A1221R, A1221V, A1221W, L1222A, L1222C, L1222E, L1222F, L1222Q, L1222R, L1222V, L1222W, S1223C, S1223F, S1223G, S1223K, S1223L, S1223V, S1224A, S1224D, S1224G, S1224L, S1224M, S1224P, S1224R, S1224W, L1225C, L1225D, L1225E, L1225F, L1225G, L1225K, L1225P, L1225T, L1225V, L1225W, T1226A, T1226G, T1226M, T1226P, T1226R, T1226S, T1226V, T1226Y, V1227A, V1227C, V1227D, V1227E, V1227G, V1227L, V1227P, V1227Q, V1227S, N1228A, N1228D, N1228F, N1228K, N1228L, N1228T, G1229A, G1229C, G1229E, G1229Q, G1229S, G1229V, T1230F, T1230H, T1230I, T1230K, T1230L, T1230P, T1230R, T1230S, T1230W, K1231F, K1231G, K1231L, K1231M, K1231P, K1231S, K1231W, V1232E, V1232K, V1232Q, V1232R, V1232S, V1232T, V1232W, S1233P, S1233W, D1234G, D1234K, D1234R, D1234V, S1235D, S1235E, S1235G, S1235L, S1235P, S1235R, S1235W, S1235Y, V1236A, V1236C, V1236G, V1236I, V1236P, V1236Q, V1236R, L1237D, L1237E, L1237R, L1237V, L1237W, A1238D, A1238E, A1238K, A1238L, A1238N, A1238P, A1238R, A1238S, A1238T, A1239D, A1239P, A1239R, G1240D, G1240L, G1240N, G1240Q, G1240S, G1240T, G1240W, S1241D, S1241G, S1241I, S1241L, S1241M, S1241P, Y1242C, Y1242E, Y1242K, Y1242R, Y1242S, Y1242W, N1243C, N1243L, N1243M, N1243P, N1243Q, N1243S, N1243T, N1243V, N1243W, T1244A, T1244D, T1244E, T1244G, T1244L, T1244Q, T1244S, T1244V, T1244W, A1246F, A1246M, A1246N, A1246P, A1246Q, A1246R, A1246S, A1246T, I1247A, I1247G, I1247M, I1247Q, I1247R, I1247S, I1247T, I1247V, I1247W, I1248A, I1248G, I1248K, I1248L, I1248R, I1248S, I1248Y, A1249E, A1249G, A1249H, A1249I, A1249R, A1249T, A1249V, D1250I, D1250K, D1250S, D1250T, D1250W, D1250Y, V1251I, V1251T, V1251W, K1252D, K1252G, K1252V, K1252W, A1253P, A1253V, E1254F, E1254G, E1254H, E1254L, E1254R, E1254V, G1255H, G1255M, G1255S, G1255V, E1256G, E1256M, E1256N, E1256R, E1256V, E1256W, G1257F, G1257K, G1257L, G1257Q, G1257R, G1257W, N1258C, N1258G, N1258H, N1258K, N1258S, A1259K, A1259L, A1259W, V1261I, V1261L, V1261P, V1261Q, V1261R, V1261T, T1262A, T1262F, T1262M, T1262Q, T1262R, V1263E, V1263G, V1263Q, V1263R, V1263T, V1263W, L1264A, L1264E, L1264H, L1264R, L1264S, L1264Y, P1265C, P1265K, P1265L, P1265R, P1265S, P1265V, P1265W, A1266F, A1266L, A1266P, A1266S, A1266V, H1267A, H1267E, H1267F, N1269A, N1269E, N1269K, N1269R, N1269S, N1269T, N1269W, V1270D, V1270E, V1270G, V1270I, V1270L, V1270T, V1270W, I1271A, I1271H, I1271Q, R1272E, R1272F, R1272M, R1272P, R1272V, V1273R, I1274F, I1274M, I1274R, T1275A, T1275L, T1275W, E1276R, E1276W, S1277L, S1277T, S1277W, E1278Q, E1278R, D1279G, D1279I, D1279R, D1279T, D1279V, D1279W, H1280C, H1280E, H1280G, H1280V, H1280W, V1281F, V1281I, V1281S, V1281W, T1282D, T1282L, T1282V, R1283A, R1283D, R1283E, R1283P, R1283W, K1284G, T1285A, T1285E, T1285F, T1285G, T1285M, T1285R, T1285Y, F1286A, F1286E, F1286P, F1286R, F1286S, F1286T, T1287C, T1287K, T1287L, T1287M, T1287Q, T1287R, T1287S, T1287W, I1288A, I1288D, I1288F, I1288G, I1288K, N1289A, N1289Q, N1289T, L1290A, L1290G, L1290R, L1290V, L1290W, G1291K, G1291P, G1291V, G1291W, G1291Y, T1292G, T1292L, T1292Y, E1293G, E1293K, E1293L, E1293S, E1293V, Q1294E, Q1294L, Q1294P, Q1294W, E1295K, F1296A, F1296G, F1296I, P1297F, A1298Y, D1301I, E1302G, E1302R, E1302S, R1303S, D1304A or D1304V.

75. The lactase variant of the preceding embodiment, wherein the variant has a decreased or an increased galactose inhibition, preferably measured as a galactose inhibition improvement factor (GI-IF) of at least 1.1 or at most 0.9, compared to the lactase of SEQ ID NO: 1.

76. The lactase variant of embodiment 1 which comprises one or more of the following substitutions, wherein each position corresponds to a position in SEQ ID NO: 1: V1D, V1F, V1H, V1K, V1L, V1R, E2D, E2G, E2Q, E2V, D3A, D3H, D3I, D3N, D3S, D3V, D3W, A4C, A4G, A4H, A4I, A4L, A4M, A4P, T5A, T5D, T5F, T5K, T5R, T5S, T5V, R6A, R6D, R6F, R6G, R6H, R6L, R6M, R6P, R6S, R6W, S7A, S7D, S7I, S7L, S7N, S7P, S7T, S7W, S9G, S9H, S9P, S9R, S9W, T10G, T10K, T10L, T10P, T10R, T10S, T10W, T11L, T11P, Q12L, Q12R, Q12V, Q12Y, M13C, M13D, M13E, M13F, M13H, M13K, M13R, M13W, S14A, S14G, S14H, S14L, S14T, S14V, S14Y, S15C, S15F, S15I, S15K, S15L, S15P, S15R, S15T, S15V, S15W, S15Y, T16A, T16C, T16I, T16N, T16S, P17A, P17C, P17D, P17E, P17I, P17L, P17N, P17R, P17S, P17T, V19A, V19F, V19G, V19I, V19K, V19L, V19N, V19S, V19W, V20C, V20F, V20G, V20I, V20K, V20L, V20M, V20N, V20P, V20Q, V20R, V20T, V20W, Y21A, Y21C, Y21D, Y21F, Y21G, Y21H, Y21M, Y21P, Y21R, Y21T, S22A, S22E, S22F, S22G, S22L, S22M, S22N, S22R, S22T, S22W, S23A, S23C, S23D, S23L, S23M, S23R, A24F, A24G, A24L, A24R, A24T, A24W, V25D, V25E, V25F, V25G, V25H, V25K, V25L, V25M, V25Q, V25R, V25S, V25T, V25W, D26C, D26I, D26L, D26M, D26T, D26V, S27A, S27C, S27F, S27G, S27H, S27P, S27Y, K28C, K28G, K28I, K28L, K28R, K28S, K28V, K28W, Q29D, Q29F, Q29G, Q29L, Q29M, Q29R, Q29S, Q29V, Q29W, N30A, N30G, N30H, N30M, N30P, N30V, N30W, N30Y, R31E, R31G, R31I, R31M, R31V, T32M, T32Q, T32R, T32S, S33C, S33E, S33H, S33K, S33N, S33Q, S33R, S33V, D34C, D34E, D34F, D34G, D34H, D34L, D34S, D34W, D34Y, F35A, F35C, F35E, F35G, F35K, F35N, F35T, F35V, D36H, D36Q, A37N, A37Q, N38C, N38G, N38S, W39G, W39S, K40C, K40D, K40F, K40G, K40I, K40M, K40N, K40P, K40W, F41A, F41C, F41G, F41I, F41Q, F41S, F41Y, M42E, M42N, M42T, L43A, L43C, L43G, L43I, L43S, L43T, L43V, S44C, S44M, S44N, S44Y, D45A, D45L, D45P, D45V, V47K, V47R, Q48S, A49C, A49D, A49H, A49R, A49S, A49T, A49V, D51G, D51I, D51K, D51M, D51P, D51V, A53C, A53G, A53L, A53R, A53S, A53V, A53W, F54M, F54S, D55C, D55F, D55G, D55H, D55M, D55N, D55P, D55S, D55V, S57A, S57C, S57E, S57G, A58D, A58G, A58I, A58M, A58N, A58Q, A58R, A58T, W59D, W59I, W59K, W59L, W59N, W59P, W59V, Q60A, Q60C, Q60E, Q60F, Q60G, Q60K, Q60L, Q60M, Q60R, Q60S, Q60V, Q60Y, Q61K, Q61P, Q61S, V62G, V62N, V62S, V62T, V62W, D63G, D63L, D63P, D63S, D63V, L64E, L64G, H66C, H66L, H66R, H66T, H66W, H66Y, D67E, Y68P, Y68V, I70A, I70H, I70K, I70P, I70R, T71C, T71E, T71G, T71H, T71K, T71L, T71N, T71P, T71Q, T71R, T71S, K73A, K73D, K73F, K73G, K73Q, K73V, Y74G, Y74K, Y74T, Y74W, S75G, S75L, S75Q, S75R, S75V, Q76G, Q76I, Q76K, Q76M, Q76P, Q76S, Q76V, Q76Y, S77C, S77D, S77E, S77F, S77G, S77H, S77I, S77K, S77L, S77M, S77R, S77T, S77V, S77W, S77Y, N78C, N78E, N78F, N78K, N78Q, N78R, N78S, N78T, E79H, E79Q, E79S, E79T, E79W, A80K, E81A, E81Q, A83E, A83T, L85A, L85C, L85D, L85F, L85M, L85N, L85S, L85V, L85W, P86E, P86G, P86H, P86N, P86Q, P86R, P86V, P86W, P86Y, G87A, G87D, G87E, G87N, G87Q, G88A, G88F, G88I, G88M, G88Q, G88S, T89C, T89G, T89H, T89K, T89L, T89M, T89N, T89P, T89W, T89Y, G90A, G90C, G90D, G90L, G90S, G90T, G90V, W91E, W91L, W91P, W91Q, W91R, W91S, W91Y, Y92F, Y92H, Y92I, Y92M, Y92N, Y92S, Y92T, Y92V, Y92W, R93A, R93D, R93F, R93H, R93I, R93L, R93N, R93T, R93V, R93Y, K94A, K94C, K94G, K94R, K94S, K94T, K94V, S95A, S95C, S95D, S95E, S95G, S95I, S95L, S95Q, S95R, F96A, F96C, F96I, F96K, F96L, F96M, F96P, F96S, F96V, F96W, T97F, T97S, T97V, I98C, I98H, I98S, I98W, R100T, D101A, D101P, D101V, L102A, L102G, L102M, L102P, L102S, G104C, K105D, K105Q, K105R, K105W, K105Y, R106K, R106P, R106V, R106W, I107A, I107F, I107G, I107Q, I107S, A108E, A108S, A108V, I109M, I109T, N110A, N110F, N110S, N110T, N110V, N110W, F111A, F111C, F111L, F111Q, F111V, D112A, D112F, D112G, D112T, G113A, G113S, V114F, V114G, V114M, V114R, Y115E, M116A, M116C, M116D, M116W, M116Y, N117K, N117R, N117T, N117W, A118K, A118P, A118Y, T119A, T119G, T119L, V120A, V120K, W121C, W121D, W121R, W121T, W121V, W121Y, F122A, F122M, F122S, F122Y, N123P, G124E, G124M, G124Q, G124R, V125D, V125E, V125I, K126E, K126V, G128A, G128D, T129E, T129V, H130A, H130C, H130Q, H130S, H130T, P131K, P131L, P131S, Y132C, Y132E, Y132S, G133E, S135E, S135P, S135V, P136R, P136Y, F137A, F137C, F137D, F137G, F137L, F137P, S138A, S138D, S138G, S138H, S138L, S138M, S138R, S138V, F139A, F139E, F139Q, F139W, D140G, D140L, D140V, L141G, L141T, T142E, T142S, T142V, K146A, G148H, G148K, G148T, G149H, G149I, G149M, G149Q, G149Y, E150A, E150C, E150G, E150L, E150N, E150R, N151L, I153A, I153Y, V154E, V154I, V154K, V154L, V154M, V154S, V155F, V157A, V157G, V157L, V157P, V157Q, V157S, E158G, E158H, E158K, E158Q, E158V, N159D, N159H, N159S, N159T, R160G, L161E, L161K, L161M, L161S, L161W, P162F, P162G, P162N, P162T, P162W, S164A, R165H, W166S, Y167A, Y167C, S168C, G169A, G169C, G169D, G169S, S170L, S170Q, G171C, G171F, G171T, I172G, I172K, I172P, I172Q, Y173A, Y173H, Y173M, Y173P, Y173S, Y173W, R174E, R174K, D175E, D175Y, V176E, V176K, V176T, T177A, T177C, T177E, T177K, T177L, L1781, L178Q, L178W, T179A, T179C, T179D, T179H, T179I, T179K, T179L, T179N, T179P, T179S, V180A, V180C, V180D, V180E, V180G, V180M, T181A, T181D, T181F, T181K, T181R, D182F, D182L, D182S, G183W, V184F, V184H, V184P, V184Q, V184R, V184S, V184W, H185G, H185L, H185R, V186A, V186E, V186G, V186N, G187A, G187D, G187H, N188E, N188R, N188S, N188V, N188W, N189A, N189E, G190C, G190F, G190H, G190Q, G190V, V191C, V191T, V191W, V191Y, I193N, I193Q, I193T, I193V, K194A, K194I, K194L, K194R, T195A, T195E, T195M, T195S, T195W, P196A, P196H, P196I, P196M, P196S, P196W, S197A, S197C, S197E, S197K, S197L, S197P, L198E, L198F, L198H, L198I, L198K, L198R, L198V, L198W, A199E, A199F, A199K, A199P, A199R, A199T, Q201C, Q201E, Q201I, Q201K, Q201M, Q201V, N202A, N202D, N202F, N202G, N202K, N202L, N202M, N202Q, N202R, N202S, N202T, N202W, G203C, G203K, G203M, G203Q, G203R, G203S, G203V, G203W, G203Y, G204A, G204C, G204D, G204K, G204R, G204S, G204Y, N205E, N205G, N205H, N205L, N205P, N205W, N205Y, V206A, V206C, V206D, V206F, V206G, V206I, V206K, V206Q, V206R, V206S, V206T, T207A, T207C, T207G, T207I, T207K, T207L, T207M, T207N, T207Q, T207R, T207W, M208A, M208S, M208T, N209C, N209D, N209G, N209K, N209L, N209Q, N209R, N209V, L210A, L210C, L210F, L210G, L210H, L210I, L210Q, L210R, L210S, L210T, L210V, T211A, T211D, T211E, T211F, T211K, T211N, T211Q, T211R, T211S, T212A, T212C, T212E, T212F, T212G, T212H, T212K, T212L, T212M, T212S, T212W, K213A, K213C, K213D, K213F, K213I, K213L, K213M, K213N, K213Q, K213R, K213S, K213T, K213V, K213Y, V214A, V214C, V214T, V214W, A215D, A215E, A215F, A215I, A215K, A215L, A215Q, A215R, A215S, A215V, N216D, N216T, N216V, D217F, D217G, D217L, D217M, D217T, D217V, T218D, T218G, T218H, K219A, K219C, K219F, K219H, K219M, A220C, A220G, A220I, A220L, A220M, A220T, A220V, A220W, A221C, A221D, A221E, A221L, A221N, A221R, A221V, A221Y, A222D, A222I, A222L, A222P, A222R, A222W, A222Y, N223A, N223E, N223F, N223G, N223K, N223L, N223M, N223R, N223S, N223T, N223V, N223W, I224G, I224Q, T225A, T225G, T225L, L226C, L226M, L226Q, K227T, Q228N, Q228R, T229A, T229C, T229D, T229G, T229H, T229M, T229N, T229Q, T229R, T229V, V230F, V230L, V230M, V230Q, V230R, V230S, F231A, F231E, F231G, F231I, F231K, F231L, F231Q, F231S, F231V, F231W, F231Y, P232G, P232H, P232L, P232M, P232R, P232S, P232T, P232V, P232W, P232Y, K233A, K233C, K233E, K233F, K233G, K233L, K233P, K233R, K233S, K233V, K233W, K233Y, G234A, G234C, G234D, G234E, G234K, G234L, G234Q, G234R, G234V, G234W, G234Y, G235C, G235F, G235H, G235I, G235K, G235M, G235Q, G235R, G235T, G235W, G235Y, K236A, K236D, K236E, K236G, K236L, K236M, K236P, K236R, K236S, K236T, K236W, K236Y, T237D, T237F, T237I, T237K, T237M, T237Q, T237R, T237S, T237V, T237Y, D238A, D238E, D238F, D238G, D238H, D238I, D238K, D238L, D238M, D238N, D238P, D238Q, D238R, A239C, A239E, A239G, A239I, A239K, A239T, A240C, A240E, A240L, A240P, A240Q, A240T, A240V, A240W, A240Y, I241T, G242K, G242L, G242M, G242P, G242T, G242Y, T243I, T243M, T243R, T243V, V244A, V244E, V244G, V244L, V244R, T245E, T245G, T245L, T245M, T245N, T245Q, T245R, T245S, T246D, T246E, T246G, T246K, T246V, A247D, A247E, A247K, A247N, A247P, A247Q, A247R, A247S, A247V, A247W, S248A, S248E, S248F, S248H, S248I, S248L, S248Q, S248T, S248Y, K249A, K249D, K249G, K249H, K249I, K249L, K249M, K249N, K249P, K249Q, K249S, K249T, K249V, K249Y, S250H, S250M, S250W, I251F, I251L, I251V, I251W, I251Y, A252C, A252E, A252F, A252H, A252I, A252P, A252R, A252S, A252W, A252Y, G254D, G254F, G254I, G254L, G254M, G254Q, G254R, G254W, A255C, A255F, A255K, A255L, A255M, A255S, A255T, A255W, A255Y, S256A, S256C, S256F, S256G, S256K, S256L, S256M, S256N, S256Q, S256R, S256V, S256W, S256Y, A257D, A257G, A257I, A257N, A257T, A257V, D258A, D258L, D258M, D258W, V259E, V259L, V259S, V259T, T260A, T260D, T260G, T260I, T260K, T260V, S261A, S261D, S261H, S261R, S261W, S261Y, T262D, T262E, T262F, T262G, T262H, T262L, T262P, T262W, I263A, I263C, I263G, I263L, I263S, I263V, T264F, T264G, T264K, T264L, T264M, T264P, T264Q, T264R, T264S, T264Y, A265G, A265I, A265K, A265R, A265S, A265V, A266D, A266E, A266G, A266K, A266L, A266M, A266P, A266Q, A266S, S267A, S267D, S267K, S267M, S267N, S267P, S267Q, S267R, S267V, P268F, P268G, P268M, P268R, P268V, P268W, P268Y, K269G, K269R, K269V, K269Y, L270D, L270M, L270N, L270R, L270V, W271T, S272E, S272G, S272K, S272L, S272N, S272T, S272W, 1273K, I273L, I273P, I273R, I273S, I273W, K274D, K274P, K274Q, K274R, N275K, N275M, N275V, N275W, N277F, N277K, N277R, L278A, L278G, L278H, L278I, L278K, L278M, L278P, L278Q, L278R, L278S, L278V, Y279M, Y279T, Y279W, T280A, T280D, T280E, T280F, T280H, T280M, T280Q, V281A, V281I, V281L, V281Q, R282E, R282F, R282H, R282I, R282K, R282N, R282S, R282T, R282V, R282W, T283M, T283R, T283V, E284A, E284D, E284F, E284H, E284L, E284M, E284N, E284Q, E284R, E284Y, V285H, V285I, V285T, L286A, L286C, L286D, L286F, L286N, L286R, L286T, L286W, L286Y, N287I, N287L, G288F, G288L, G288S, V291C, V291D, V291F, V291G, V291H, V291L, V291P, V291S, V291T, V291Y, L292A, L292D, L292E, L292H, L292Q, L292S, L292V, D293C, D293E, D293F, D293I, D293S, D293V, D293W, T294C, T294G, T294M, T294Q, T294S, Y295F, Y295G, Y295I, Y295L, Y295R, Y295S, Y295W, D296F, D296H, D296K, D296L, D296N, D296R, D296V, T297G, T297I, T297Q, T297R, T297S, E298G, E2981, E298L, E298M, E298R, E298T, Y299F, Y299S, F301C, F301L, R302E, R302I, R302K, R302M, R302N, R302T, W303A, W303C, W303D, W303F, W303S, W303T, T304D, T304E, T304I, T304K, T304P, T304S, T304W, G305E, G305F, G305L, G305M, G305N, G305P, G305T, G305W, F306D, F306K, F306L, F306S, D307A, D307E, D307F, D307G, D307L, D307M, D307N, D307Q, D307S, D307V, D307W, A308C, A308E, A308G, A308I, A308M, A308Y, T309C, T309D, T309E, T309I, T309K, T309M, T309S, T309V, S310A, S310C, S310G, S310H, S310I, S310L, S310M, S310N, S310R, S310V, G311E, G311F, G311L, G311Q, G311R, G311V, F312H, F312L, F312R, F312V, L314A, L314C, L314T, L314V, L314Y, N315S, G316A, G316C, G316N, G316R, G316S, G316W, G316Y, E317D, E317H, E317I, E317K, E317V, E317W, E317Y, K318C, K318E, K318F, K318G, K318H, K318L, K318M, K318N, K318P, K318Q, K318V, K320C, K320E, K320F, K320I, K320L, K320R, K320T, K320V, K320W, L321C, L321G, L321H, L321M, L321V, L321Y, K322C, K322F, K322I, K322N, K322P, K322Q, K322R, K322S, V324E, V324G, V324T, S325D, S325G, S325T, M326E, M326G, M326S, M326T, M326V, M326Y, H328C, H328D, H328F, H328G, H3281, H328L, H328M, H328R, H328T, D329S, D329T, G331V, A335C, A335G, A335L, A337D, A337E, A337G, A337L, A337Q, N338P, N338R, N338V, R340A, R340C, R340L, A341M, A341S, I342A, I342G, I342K, I342P, E343A, E343H, E343N, E343R, E343S, E343T, E343Y, R344G, R344L, R344Y, Q345F, Q345G, Q345K, Q345N, Q345S, V346A, V346C, V346F, V346I, V346L, V346S, E347A, E347C, E347D, E347F, E347I, E347R, E347S, I348A, I348D, I348G, I348M, I348Q, I348R, L349A, L349E, L349Q, L349S, L349V, Q350G, Q350N, Q350W, K351N, K351R, K351V, K351W, M352G, M352L, M352T, M352W, G353K, V354E, V354G, V354S, V354W, N355H, N355M, N355R, N355W, I357T, T359E, T359L, T360V, N362G, N362S, N362T, P363A, P363D, P363I, P363L, P363M, P363S, P363V, A364C, A364E, A364F, A364G, A364I, A364M, A364P, A364V, A364W, A365C, A365E, A365I, A365P, A365V, A365W, K366A, K366D, K366E, K366I, K366L, K366M, K366P, K366S, K366V, A367C, A367I, A367N, A367Q, L368A, L368E, L368Q, L368S, L368V, L368Y, I369A, I369D, I369E, I369G, I369K, I369M, I369R, I369V, I369W, D370C, D370L, D370Q, D370R, D370S, D370T, V371D, V371F, V371G, V371I, V371L, V371Q, V371S, C372P, N373G, N373L, N373R, E374K, E374L, E374R, K375D, K375I, K375N, K375Q, K375S, G376A, G376S, V377A, V377M, V377T, L378I, L378P, L378W, L378Y, V379A, V379C, V379M, V379N, V380M, V380P, V380S, E381A, E381C, E381G, E381L, E381Q, E381T, V383A, V383K, V383L, M386G, M386N, M386Q, M386S, M386V, W387H, W387L, N388A, N388E, N388L, N388R, R389A, R389C, R389E, R389K, R389M, R389N, R389Q, R389S, R389T, R389V, S390C, S390D, S390G, S390H, S390N, S390P, S390Q, S390T, S390V, K391E, N392D, G393A, G393E, G393N, G393R, G393S, G393V, N394L, T395A, T395C, T395F, T395H, T395I, T395M, T395N, T395Q, T395S, T395W, E396K, E396L, E396M, E396V, E396W, Y398M, Y398N, G399S, K400A, K400C, K400D, K400E, K400M, K400N, K400P, K400Q, K400S, K400T, K400V, W401F, W401H, W401K, W401L, W401M, W401R, F402T, F402W, F402Y, G403A, G403D, G403H, G403K, G403P, G403Q, G403S, G403T, G403V, G403Y, Q404F, Q404H, Q404L, Q404M, Q404P, Q404R, Q404S, Q404V, A405C, A405E, A405H, A405K, A405P, A405R, A405V, I406C, I406D, I406N, A407C, A407G, A407M, A407Q, A407S, A407T, A407W, G408D, G408I, G408M, G408N, G408W, D409N, D409W, N410C, N410R, N410Y, A411E, A411N, A411R, A411S, A411V, V412M, V412S, L413D, L413E, L413F, L413I, L413P, L413T, G414A, G414C, G414M, G414N, G414R, G414T, G414W, G415A, G415Q, G415R, D416I, D416M, D416R, D416T, D416Y, K417C, K417F, K417G, K417R, K417T, D418L, D418P, D418R, D418Y, E419M, E419R, E419W, T420E, T420F, T420G, T420K, T420R, T420V, W421L, W421Q, W421S, A422P, A422T, A422V, K423D, K423L, K423M, K423R, F424C, F424L, F424N, F424T, D425E, L426C, L426M, L426Q, T427D, T427F, T427G, T427K, T427M, T427P, T427Q, T427R, T427S, T427W, S428F, S428K, S428W, T429D, T429P, I430C, I430D, I430E, I430L, I430M, I430Q, I430S, I430T, I430W, N431D, N431E, N431G, N431L, N431M, N431R, N431V, N431Y, R432A, R432E, R432F, R432G, R432N, R432Q, R432V, R432Y, D433C, D433G, D433H, D433I, D433P, D433Q, D433W, R434L, R434M, R434N, R434P, R434S, R434T, R434V, N435E, N435F, N435H, N435K, N435L, N435M, N435R, N435V, N435W, A436C, A436D, A436E, A436G, A436I, A436L, A436M, A436Q, A436S, P437A, P437D, P437K, P437L, P437Q, P437R, P437S, P437V, P437W, S438G, V439C, V439E, V439G, V439I, V439K, V439Q, V439T, V439Y, I440C, I440D, I440F, I440K, I440P, I440R, I440S, I440T, I440V, I440W, M441A, M441E, M441G, M441Q, M441R, M441T, M441V, W442E, W442G, W442M, W442P, W442Q, W442R, S443C, S443D, S443G, S443M, S443Q, S443Y, L444C, L444D, L444E, L444F, L444G, L444H, L444K, L444Q, L444V, L444W, G445A, G445C, G445V, N446D, N446T, M448A, M448C, M448D, M448E, M448I, M448L, M448P, M448Q, M448S, M448V, M448W, M449D, M449E, M449F, M449T, M449V, M449W, M449Y, E450F, E450S, E450T, E450V, E450W, G451C, G451F, G451L, G451P, G451V, G451W, I452G, I452K, I452L, I452M, I452Q, I452S, I452V, S453C, S453F, S453G, S453H, S453L, S453M, S453N, S453P, S453Q, S453R, S453V, G454L, G454W, S455A, S455E, S455K, S455M, S455P, S455R, S455V, S455W, V456A, V456D, V456E, V456F, V456K, V456L, V456W, S457E, S457H, S457K, S457L, S457M, S457P, S457Q, S457T, S457V, G458A, G458C, G458F, G458L, G458P, G458Q, G458S, G458V, G458W, F459A, F459C, F459E, F459G, F459N, F459R, F459S, F459T, F459W, P460C, P460M, P460Q, P460W, P460Y, A461D, A461G, A461M, A461N, A461Q, A461S, A461V, A461Y, T462C, T462E, T462F, T462L, T462M, T462S, S463G, S463K, S463Q, S463R, S463T, S463V, A464E, A464H, A464L, A464M, A464P, A464V, A464W, K465C, K465F, K465G, K465L, K465Q, K465R, K465V, K465W, K465Y, L466A, L466C, L466D, L466E, L466F, L466G, L466M, L466P, L466Q, L466S, L466V, L466Y, V467A, V467C, V467D, V467E, V467G, V467T, V467W, A468D, A468E, A468F, A468K, A468L, A468P, A468S, A468V, A468W, W469A, W469C, W469D, W469G, W469L, W469M, W469R, W469V, W469Y, T470E, T470L, T470M, T470Q, K471F, K471G, K471Q, K471W, K471Y, A472G, A472Y, A473E, A473M, A473P, D474A, D474C, D474E, D474K, D474M, D474R, D474W, S475E, S475F, S475Q, S475T, S475V, T476C, T476L, T476S, R477A, R477C, R477G, R477L, P478A, P478D, P478G, P478L, P478V, M479G, M479I, M479R, M479W, T480C, T480G, T480Q, K485E, K485R, K487A, K487C, K487F, K487G, K487N, K487S, K487W, A488C, A488G, A488H, A488L, A488N, A488S, A488V, N491A, N491E, N491W, E492A, E492W, S493E, S493G, S493H, S493L, S493M, S493Q, S493V, N494A, N494I, N494M, N494R, N494V, T495K, T495R, T495V, T495W, M496A, M496F, M496T, G497D, D498A, D498C, D498M, D498S, N499K, N499R, N499T, N499Y, L500A, L500E, L500N, L500V, T501C, T501G, T501M, A502L, A502Q, N503A, N503E, N503M, N503S, G504H, G504K, G504P, G505A, G505D, G505E, G505H, G505L, G505N, G505R, G505S, G505V, V506C, V506D, V506E, V506G, V506I, V506L, V506P, V506R, V506S, V506T, V506W, V507A, V507F, V507G, V507L, V507N, V507P, V507R, V507S, V507T, G508C, G508E, T509A, T509D, T509E, T509I, T509K, T509M, T509Q, T509S, T509V, T509Y, N510A, N510F, N510I, N510Q, Y511A, Y511K, S512C, S512E, S512F, S512G, S512I, S512M, S512Q, S512T, S512V, S512Y, D513C, D513G, D513K, D513L, D513M, D513P, D513Q, D513R, D513W, G514F, G514L, G514N, G514P, G514R, A515C, A515D, A515E, A515F, A515G, A515K, A515R, A515S, N516C, N516E, N516G, N516I, N516M, N516Q, N516S, N516T, N516V, Y517G, Y517I, Y517N, D518Q, D518Y, K519C, K519E, K519F, K519G, K519I, K519L, K519M, K519N, K519Q, K519S, K519T, I520A, I520C, I520G, I520H, I520M, I520N, I520Q, I520S, I520T, I520W, I520Y, R521A, R521C, R521K, R521N, R521Q, R521V, T522G, T522I, T522N, T523A, T523M, H524R, H524V, P525D, P525G, P525R, P525T, P525V, S526G, S526I, S526W, W527A, W527E, W527G, W527H, W527N, W527R, W527S, W527V, A528C, A528E, A528G, A528I, A528L, I529F, I529G, I529L, I529Y, Y530A, Y530M, G531E, G531S, G531T, T534A, T534I, T534Q, A535E, A535G, A535I, A535M, A537D, A537M, A537P, A537S, I538H, I538M, N539G, N539W, S540E, S540G, S540M, G542E, G542Q, G542S, G542T, I543V, I543W, N545G, N545I, N545Q, N545S, R546A, R546C, R546L, R546P, R546S, T547A, T547D, T547H, T547K, T547N, T547S, T548D, T548E, T548F, T548K, T548L, T548P, T548W, G549D, G549F, G549P, G549W, G550Q, G550R, G550S, A551D, A551I, A551Q, S553C, S553F, S553N, S553P, S553R, S553T, S553V, S554F, S554N, S554R, S554T, S554V, S554W, D555E, D555P, D555S, K556A, K556C, K556R, K556W, Q557F, Q557R, Q557S, L558E, L558H, L558I, L558P, T559A, T559G, T559I, T559Q, T559V, T559Y, S560P, S560V, Y561R, N563R, N563S, S564A, S564C, S564F, S564W, A565C, A565D, A565E, A565F, A565G, A565I, A565L, A565M, A565R, A565S, A565T, A565V, A565W, G567N, G567Q, G567V, A570G, A570K, A570L, A570M, A570S, A570T, A570W, V571W, A572S, A572W, S573G, S573K, S573Y, S574K, S574Q, S574V, S574W, A575D, A575M, A575V, W576A, W576F, W576V, W576Y, Y577G, Y577I, Y577L, Y577R, D578E, D578M, D578N, D578T, V579E, V579G, V579L, V579T, V580A, V580D, V580E, V580K, V580L, V580S, Q581F, Q581G, Q581P, Q581R, Q581S, Q581T, Q581Y, R582A, R582D, R582G, R582I, R582L, R582Y, D583V, D583W, F584E, F584I, F584W, V585I, V585M, V585Q, A586C, A586D, A586H, A586K, G587A, G587C, T588C, T588D, T588G, T588I, T588L, T588M, T588P, Y589A, Y589I, Y589Q, Y589V, Y589W, V590A, V590H, V590I, W591F, T592C, T592L, T592Q, T592S, G593C, G593I, F594C, F5941, F594L, F594M, D595E, D595Q, D595S, L597C, L597D, L597E, L597T, G598N, P600A, P600E, P600G, P600S, N604E, N604S, G605R, T606S, G607A, G607Q, G607S, S608E, S608I, S608M, S608N, S608Q, S608T, S608V, G609A, G609H, G609L, G609N, G609R, G609S, A610D, A610F, A610M, A610T, V611K, G612N, G612T, G612V, S613A, W614A, W614F, W614P, P615L, P615S, P615V, S616A, S616W, N619A, N619I, N619L, N619S, S620G, Y621W, I624A, I624V, V625A, V625E, V625F, V625M, V625Y, T627F, T627G, T627K, T627Q, A628C, A628D, A628N, G629T, F630A, F630C, F630D, F630G, F630S, F630Y, P631A, P631D, P631G, P631H, P631S, P631V, P631Y, K632C, K632D, K632G, K632T, D633G, T634A, T634E, T634F, T634S, T634V, Y635R, Y636K, F637C, F637G, F637I, F637S, F637T, F637V, Y638A, Y638W, Q639D, Q639N, Q639R, S640C, S640D, Q641T, W642I, N643R, D644C, D644G, D644Y, D645S, D645V, V646C, V646L, V646N, V646R, V646S, H647G, H647V, T648C, L649V, H650E, H650F, H650R, I651F, I651T, I651V, L652C, L652D, L652V, L652W, P653Q, A654D, A654K, A654M, A654R, W655F, W655R, N656A, N656K, N656V, E657K, E657R, E657V, V659D, V659N, A661E, A661G, A661H, A661K, A661L, A661M, A661Q, A661W, K662H, K662S, K662V, K662W, K662Y, N667L, N667R, P669A, P669E, P669F, P669L, P669R, P669T, P669W, V670C, V672L, Y673E, Y673G, Y673I, Y673R, Y673S, Y673W, T674C, T674D, T674G, T674H, T674M, T674Q, D675A, D675E, D675P, D675Q, D675S, D675V, D675Y, A676C, A676E, A676G, A676K, A676L, A676P, A676S, A676W, A677E, A677G, A677L, A677R, A677T, A677V, A677Y, K678A, K678C, K678T, K678V, V679G, V679Q, V679S, V679T, V679Y, K680A, K680E, K680G, K680H, K680I, K680L, K680N, K680Q, K680S, K680V, K680W, L681E, L681F, L681G, L681M, L681S, L681T, Y682D, Y682E, Y682I, Y682M, Y682S, Y682V, Y682W, F683H, F683L, F683M, F683Q, F683R, F683W, T684A, T684D, T684G, T684L, T684R, T684S, T684V, P685A, P685E, P685I, P685L, P685W, K686A, K686E, K686I, K686M, K686T, K686V, G687A, G687K, G687N, G687P, G687Q, G687R, S688C, S688D, S688E, S688G, S688K, S688L, S688P, S688T, T689A, T689D, T689E, T689G, T689L, T689P, T689S, T689W, T689Y, E690D, E690M, E690P, E690T, E690V, K691E, K691H, K691N, K691P, K691R, K691S, R692G, R692H, R692I, R692L, R692P, R692S, R692T, R692V, R692W, L693A, L693M, L693P, I694L, I694W, G695C, G695K, G695L, G695R, G695W, E696A, E696L, E696R, K697A, K697E, K697G, K697R, K697V, K697W, S698C, S698D, S698E, S698I, S698L, S698M, S698P, S698Q, S698R, S698T, T700A, T700C, T700D, T700E, T700G, T700K, T700Y, K701A, K701D, K701E, K701G, K701H, K701L, K701M, K701P, K701S, K701W, T703E, T703I, T703K, T703W, T704M, T704R, T704Y, A705C, A705D, A705E, A705K, A705N, A705P, A705R, A705V, A705W, A706C, A706G, A706T, A706W, A706Y, Y708C, Y708F, Y708G, Y708K, Y708L, Y708P, Y708T, T709M, Y710C, Y710D, Y710E, Y710G, Y710M, Y710N, Y710T, Y710V, Y710W, Q711A, Q711D, Q711G, Q711L, Q711M, Q711T, Q711Y, V712G, V712M, V712P, V712Q, V712R, Y713A, Y713E, Y713G, Y713L, Y713Q, E714D, E714H, E714I, E714K, E714M, E714N, E714R, E714S, E714V, E714Y, G715K, A716C, A716G, A716L, A716M, A716P, A716R, A716V, D717C, D717G, D717L, D717S, K718A, K718L, K718Q, K718T, K718W, K718Y, D719E, D719L, D719P, D719S, D719T, D719V, S720A, S720M, S720R, S720Y, T721D, T721H, T721K, T721L, T721N, T721P, T721Q, T721S, T721V, T721W, T721Y, A722Q, A722T, H723G, H723P, K724A, K724G, K724R, N725A, N725D, N725I, M726A, M726D, M726E, M726K, M726Q, M726V, M726W, Y727L, Y727T, L728C, L728K, L728Q, L728S, L728T, L728W, T729A, T729E, T729M, W730A, N731A, N731E, N731Q, N731S, N731Y, V732G, V732P, V732R, V732W, P733A, P733R, P733W, W734D, W734G, W734R, W734V, A735Q, A735R, A735S, A735T, A735W, E736S, G737F, G737K, G737N, G737Y, T738G, T738L, T738S, I739E, I739K, I739M, I739W, I739Y, S740D, S740E, S740F, S740H, S740L, A741I, A741P, A741S, A741V, E742D, E742M, E742Q, E742V, A743C, A743E, A743H, A743I, A743L, Y744A, Y744E, Y744I, Y744K, Y744L, Y744R, D745C, D745F, D745N, D745R, E746A, E746C, E746K, E746T, N747E, N747F, N747G, N747P, N747R, N748S, R749H, R749M, R749S, R749T, L750G, L750M, L750P, L750Q, L750S, I751C, I751H, I751Q, I751S, P752A, P752C, P752H, P752L, P752S, P752V, P752Y, E753Q, G754H, G754I, G754P, S755C, S755I, S755T, T756E, T756N, T756P, T756Q, T756S, T756W, E757A, G758A, G758V, N759D, N759R, N759S, N759V, A760G, A760N, A760P, A760Q, S761A, S761K, S761Q, V762D, V762G, V762K, V762W, T765A, T765P, T765R, T765W, G766M, G766S, A768H, A769F, A769G, A769I, A769N, A769V, A769Y, K770H, L771A, L771I, K772A, K772C, K772P, K772V, K772W, A773C, A773G, A773H, A773M, A773R, A773S, A773V, D774A, D774R, A775L, A775V, A775W, D776I, D776L, D776R, D776S, D776V, R777D, R777E, R777G, R777H, R777P, R777S, R777T, K778A, K778C, K778F, K778G, K778L, K778N, K778R, T779C, T779I, I780V, T781A, T781C, T781E, T781F, T781G, T781M, T781P, T781R, T781Y, A782C, A782E, A782K, A782N, A782P, A782Q, A782Y, D783A, D783C, D783E, D783R, G784A, G784F, G784L, G784S, G784T, K785C, K785I, K785S, K785V, K785W, K785Y, D786S, D786V, L787D, L787K, L787P, L787T, L787Y, S788A, S788G, S788I, Y789C, Y789D, Y789I, Y789V, I790A, I790C, I790F, I790R, I790V, E791A, E791D, E791F, E791M, E791S, E791T, E791V, E791W, V792C, V792G, V792L, V792S, V792Y, D793C, D793F, D793H, D793K, D793N, D793Y, V794C, V794D, V794L, V794Q, V794T, V794W, T795P, T795Y, D796M, D796Q, D796S, D796T, A797H, A797K, N798G, N798I, N798P, N798Q, G799D, G799K, G799L, G799M, G799Q, G799Y, H800A, H800F, H800G, H800L, H800S, H800V, I801C, I801E, I801W, V802E, V802I, V802P, V802S, V802Y, P803A, P803F, P803G, P803K, P803S, P803Y, D804E, D804G, D804K, D804N, D804S, A805C, A805F, A805G, A805I, A805N, A805P, A806F, A806I, A806Q, N807F, N807Q, N807V, N807W, R808C, R808F, R808G, R808I, R808N, R808P, R808Q, V809A, V809C, V809L, V809M, V809P, T810L, T810P, T810Q, T810R, T810Y, F811L, F811Y, D812E, D812F, D812I, D812Q, V813F, V813H, V813T, V813W, V813Y, K814G, K814H, K814I, K814L, K814P, G815A, G815F, G815M, G815P, G815V, A816C, A816D, A816F, A816I, A816N, A816V, A816W, G817C, G817H, G817I, G817N, G817S, K818D, K818F, K818L, K818Q, K818R, K818S, K818V, K818W, K818Y, L819F, L819W, V820C, V820F, V820I, V820K, V820R, V820W, G821A, G821C, G821E, G821F, G821I, G821K, G821M, G821N, G821V, G821Y, V822A, V822D, V822E, V822T, D823E, N824A, N824C, N824G, N824Q, G825A, S826A, S826F, S826G, S826I, S826L, S826R, S826W, S827C, S827Q, P828C, P828G, P828I, P828L, P828Y, D829C, D829I, D829S, D829T, D829V, H830E, H830G, H830M, H830P, H830Q, H830R, H830V, D831A, D831F, D831G, D831I, D831M, D831P, D831R, D831V, S832E, S832F, S832G, S832L, S832M, S832P, S832R, S832V, S832W, Y833C, Y833D, Y833E, Y833I, Y833K, Y833N, Y833P, Y833V, Q834F, Q834G, Q834M, A835D, A835E, A835F, A835H, A835K, A835W, D836C, D836E, D836H, D836Q, D836R, D836S, D836T, D836V, D836W, D836Y, N837D, N837F, N837G, N837H, N837L, N837P, N837T, R838D, R838F, R838G, R838K, R838M, R838N, R838S, R838W, K839A, K839C, K839D, K839E, K839G, K839L, K839N, K839P, A840G, A840I, A840P, A840V, F841C, F841D, F841I, F841K, F841W, S842A, S842L, S842M, G843A, G843C, K844A, K844G, K844L, K844W, K844Y, V845N, V845W, L846G, L846I, L846M, L846S, A847L, A847T, I848M, V849A, V849L, V849S, V849T, Q850C, Q850G, Q850I, Q850L, Q850T, Q850V, Q850Y, S851C, S851D, S851E, S851L, S851T, T852D, T852G, T852L, K853N, K853P, K853Q, K853V, E854C, E854I, E854M, E854R, A855K, A855V, A855Y, E857P, E857V, I858D, I858E, I858F, I858G, I858K, I858M, I858P, I858Q, I858Y, T859V, V860T, V860Y, T861F, T861I, T861Q, T861V, T861W, A862C, A862P, A862V, K863F, K863I, K863L, K863N, K863W, A864E, A864H, A864K, A864L, D865A, D865G, G866H, G866R, L867W, S870E, S870M, S870R, S870T, T871P, V872C, V872G, K873G, K873Y, I874G, A875R, T877A, V879P, V879S, P880S, G881W, T882A, T882M, T882R, S883L, T884A, E885V, K886E, K886L, K886V, K886W, T887A, T887D, T887F, T887G, T887N, T887R, T887V, V888A, V888D, V888G, R889G, Y892D, Y892P, Y892R, Y893E, Y893G, S894D, S894G, R895M, N896M, Y897V, Y898T, V899G, K900E, K900G, T901G, T901Q, T901R, T901V, T901Y, G902A, G902D, G902F, G902L, G902P, G902Q, G902R, G902S, G902W, N903D, N903I, K904D, K904E, K904M, K904N, K904R, K904S, K904V, K904W, P905A, P905C, P905R, P905V, P905W, P905Y, I906A, I906D, I906S, I906T, I906W, I906Y, L907F, L907S, L907Y, P908C, P908D, P908G, P908I, P908L, P908M, P908T, S909E, S909F, S909G, S909W, S909Y, D910C, D910I, D910S, D910W, V911A, V911S, E912A, E912K, E912L, E912T, E912V, V913G, V913Q, V913R, V913W, R914A, R914E, R914F, R914I, R914K, R914V, R914W, R914Y, Y915A, Y915C, Y915G, Y915I, Y915M, Y915Q, Y915V, S916G, D917C, D917E, D917F, D917L, D917M, D917Q, D917S, G918E, G918H, G918T, G918V, G918W, T919D, T919K, T919Q, T919W, T919Y, S920C, S920E, S920M, S920P, S920R, S920V, S920W, D921C, D921P, D921Q, D921V, R922A, R922G, R922M, R922V, R922W, Q923A, Q923C, Q923E, Q923L, Q923M, Q923V, Q923W, N924A, N924L, N924P, N924Q, N924S, N924W, V925A, V925C, V925E, V925G, V925K, V925N, V925S, V925W, T926G, T926P, T926R, T926S, T926V, T926W, W927C, W927G, W927P, D928A, D928E, D928H, D928L, D928Q, A929C, A929P, A929V, V930A, V930E, V930I, V930K, V930M, V930T, S931G, S931P, S931R, D932F, D932R, D932S, D932T, D932V, D933I, D933R, D933S, Q934S, Q934V, I935A, I935C, I935D, I935E, I935L, I935P, I935V, I935W, A936I, A936L, A936Q, A936R, A936Y, K937G, K937I, K937M, K937P, K937Q, K937R, K937V, A938C, A938H, A938N, A938T, A938V, A938W, G939D, G939K, S940C, S940E, S940M, S940R, S940T, S940V, S940W, F941C, F941M, F941W, S942A, S942E, S942K, S942L, S942P, S942T, S942V, V943A, V943G, V943H, V943Q, V943R, A944D, A944G, A944H, A944P, A944R, A944V, G945E, G945P, G945T, T946A, T946E, T946G, T946L, T946P, T946V, T946W, V947G, V947H, V947L, V947M, V947P, V947R, V947T, A948C, A948I, A948R, A948W, G949A, G949F, G949V, Q950D, Q950G, Q950K, Q950M, Q950W, K951D, K951G, K951P, K951Q, K951S, K951W, K951Y, I952H, I952Q, S953F, S953M, S953N, S953R, S953W, V954D, V954L, V954Q, V954S, V954T, R955A, R955C, R955E, R955K, R955Q, R955W, V956A, V956D, V956G, V956H, V956I, V956M, V956Q, V956W, T957D, T957S, T957W, M958D, M958I, M958K, I959A, I959L, I959S, I959V, I959Y, D960G, D960H, D960L, D960P, D960S, D960W, E961D, E961F, E961K, E961P, E961S, E961T, I962A, I962C, I962D, I962G, I962K, I962N, I962Q, I962T, G963C, G963E, G963L, G963P, A964C, A964E, A964H, L965C, L965E, L965G, L965K, L965M, L965P, L965Q, L965S, L965V, L965Y, L966A, L966G, L966H, L966K, L966N, L966P, L966Q, L966S, L966T, L966V, N967C, N967D, N967I, N967L, N967M, N967P, N967S, N967T, N967V, Y968G, Y968L, Y968Q, Y968V, S969C, S969D, S969G, S969H, S969I, S969L, S969M, S969P, S969Q, S969Y, A970I, A970L, S971E, S971F, S971G, S971H, S971V, S971W, P973C, P973D, P973K, P973N, P973Q, P973R, P973V, P973W, P973Y, V974C, V974E, V974G, V974N, V974T, V974Y, G975D, G975F, G975K, G975L, G975Q, G975V, T976D, T976F, T976G, T976K, T976L, T976P, T976S, T976Y, P977A, P977C, P977K, P977R, P977T, P977Y, A978F, A978G, A978M, A978N, A978P, A978R, A978S, A978Y, V979G, V979N, V979R, V979Y, L980A, L980F, L980H, L980I, L980K, L980N, L980Q, L980T, L980Y, P981L, P981M, G982A, G982H, G982M, G982P, G982Q, G982W, R984P, R984S, P985E, P985F, P985H, P985K, P985L, P985W, A986C, A986E, A986F, A986I, A986K, A986L, A986M, A986N, A986S, A986W, V987A, V987C, V987F, V987I, V987K, V987L, V987Q, V987T, L988A, L988C, L988E, L988G, L988H, L988M, L988Q, L988R, L988S, L988V, L988Y, P989A, P989C, P989D, P989G, P989H, P989I, P989M, P989N, P989Q, P989W, D990F, D990P, D990S, D990W, G991C, G991F, G991H, G991K, G991P, G991Y, T992E, T992H, T992M, T992N, T992Y, V993D, V993G, V993N, V993S, T994I, T994S, T994V, S995E, S995L, S995R, S995V, A996Q, A996R, A996V, N997A, N997C, N997E, N997K, N997L, N997S, N997V, N997W, N997Y, F998M, F998W, A999F, A999G, A999L, A999M, A999R, A999S, V1000C, V1000L, V1000M, V1000N, V1000P, V1000W, D1001G, D1001K, D1001L, D1001M, D1001Q, D1001S, D1001T, D1001V, D1001Y, W1002A, W1002D, W1002E, W1002H, W1002N, W1002P, W1002Q, W1002S, T1003F, T1003G, T1003L, T1003N, T1003P, T1003R, T1003S, T1003W, T1003Y, K1004D, K1004E, K1004F, K1004G, K1004H, K1004M, K1004P, K1004R, K1004S, K1004V, P1005I, P1005N, P1005Q, P1005V, P1005Y, A1006C, A1006I, A1006N, A1006P, A1006S, A1006V, A1006W, A1006Y, D1007C, D1007L, D1007P, D1007V, T1008G, V1009G, V1009S, Y1010A, Y1010P, Y1010R, Y1010T, N1011A, N1011S, N1011T, N1011W, T1012E, T1012H, T1012I, T1012Q, T1012Y, A1013D, A1013K, A1013Q, A1013T, A1013V, G1014E, G1014I, G1014L, G1014M, G1014V, G1014W, G1014Y, T1015A, T1015F, T1015G, T1015V, V1016C, V1016D, V1016P, K1017E, K1017G, V1018I, V1018K, V1018L, V1018M, V1018R, V1018S, V1018W, T1021C, T1021E, T1021F, T1021G, T1021K, T1021L, T1021S, T1021V, A1022H, A1022L, A1022S, A1022Y, T1023D, T1023M, T1023Q, T1023R, V1024E, V1024G, V1024H, V1024K, V1024N, V1024R, V1024S, V1024W, G1026E, G1026H, G1026L, G1026R, G1026S, G1026V, G1026Y, K1027C, K1027N, K1027Q, K1027R, K1027V, E1028G, E1028S, E1028T, F1029I, F1029K, F1029L, F1029P, F1029V, F1029W, F1029Y, K1030D, K1030F, K1030H, K1030L, K1030M, K1030W, V1031H, V1031K, V1031Y, A1033G, A1033S, A1033V, T1034G, T1034H, T1034N, T1034W, I1035D, I1035G, I1035Q, R1036G, R1036L, R1036T, R1036Y, V1037C, V1037F, V1037P, V1037Q, Q1038A, Q1038D, Q1038K, R1039S, R1039V, S1040A, S1040M, S1040N, S1040R, S1040W, Q1041P, V1042N, T1043F, T1043G, T1043N, T1043R, I1044A, I1044L, G1045S, S1046I, S1046M, S1047D, V1048C, V1048F, V1048G, V1048I, V1048M, V1048Q, G1050L, G1050S, G1050V, N1051A, N1051E, N1051K, A1052C, A1052K, A1052M, A1052P, A1052R, L1053A, L1053W, R1054C, R1054L, R1054N, L1055R, L1055T, Q1057A, Q1057E, Q1057P, Q1057R, N1058R, N1058S, N1058V, N1058W, I1059W, P1060G, P1060N, P1060Q, P1060S, P1060T, A1061E, A1061G, A1061K, A1061W, D1062A, D1062F, D1062G, D1062I, D1062L, D1062M, D1062P, D1062S, K1063D, K1063M, Q1064C, Q1064M, Q1064R, Q1064T, Q1064V, S1065A, S1065C, S1065E, S1065G, S1065T, S1065W, D1066A, D1066G, D1066M, D1066V, D1066W, T1067G, T1067M, L1068C, L1068E, L1068P, L1068Q, L1068Y, D1069G, D1069K, D1069R, D1069W, A1070P, A1070T, I1071M, I1071R, I1071W, K1072E, K1072G, K1072P, K1072Q, K1072S, D1073F, D1073L, D1073M, D1073P, D1073W, G1074I, G1074L, G1074R, S1075C, S1075G, S1075I, S1075L, S1075V, T1076C, T1076E, T1076H, T1076Q, T1076S, T1077K, T1077L, T1077R, V1078D, V1078E, V1078L, V1078W, D1079G, D1079L, N1081D, N1081E, N1081G, T1082A, T1082C, T1082E, T1082F, T1082G, T1082K, T1082N, T1082S, G1083E, G1083F, G1083L, G1083P, G1083S, G1084C, G1084M, G1084V, G1084W, G1084Y, G1085A, G1085P, G1085R, G1085S, A1086H, A1086K, A1086Q, A1086R, A1086T, N1087A, N1087E, N1087I, N1087R, N1087V, N1087W, P1088D, P1088E, P1088G, P1088R, P1088W, S1089C, S1089E, S1089G, S1089K, S1089Q, S1089R, S1089V, A1090F, A1090G, A1090I, A1090K, W1091A, W1091E, W1091G, W1091H, W1091T, W1091V, W1091Y, T1092A, T1092E, T1092G, T1092K, T1092Q, T1092S, T1092V, N1093A, N1093G, N1093L, N1093P, N1093Q, N1093T, N1093V, W1094D, W1094E, W1094P, W1094R, W1094T, A1095P, A1095R, A1095T, A1095W, Y1096A, Y1096D, Y1096H, Y1096L, Y1096R, S1097D, S1097E, S1097K, S1097L, S1097T, S1097W, K1098D, K1098F, K1098G, K1098Q, K1098S, A1099C, A1099D, A1099F, A1099S, A1099V, A1099W, G1100D, G1100E, G1100H, G1100M, G1100N, G1100T, H1101K, H1101L, H1101Q, H1101R, H1101V, N1102E, N1102F, N1102H, N1102K, N1102L, N1102Q, N1102R, N1102T, T1103A, T1103E, T1103H, T1103S, T1103W, A1104I, A1104K, A1104R, E1105L, E1105S, I1106T, I1106V, T1107C, T1107M, T1107R, T1107S, F1108D, F1108K, F1108L, F1108T, F1108W, E1109A, E1109D, E1109L, E1109W, A1111G, A1111S, E1113D, E1113G, E1113P, E1113V, Q1114E, Q1114I, Q1114L, Q1114R, Q1114S, Q1114T, Q1114V, Q1115A, Q1115K, Q1115L, Q1115P, Q1115T, Q1115W, L1116D, L1116G, L1116H, L1116K, L1116V, L1116W, G1117E, G1117I, G1117M, G1117R, G1117S, G1117T, G1117V, G1117W, Q1118A, Q1118M, Q1118S, Q1118T, Q1118W, I1119D, I1119E, I1119G, I1119N, I1119S, V1120N, V1120S, V1120T, M1121G, M1121K, M1121N, M1121P, M1121S, M1121V, M1121Y, Y1122A, Y1122C, Y1122I, Y1122K, Y1122R, Y1122V, Y1122W, F1123E, F1123H, F1123R, F1123T, F1124E, F1124R, F1124V, F1124W, R1125D, R1125E, R1125F, R1125K, R1125T, R1125V, R1125W, D1126H, D1126K, D1126L, D1126R, S1127F, S1127I, S1127K, S1127M, S1127Q, S1127T, S1127W, N1128A, N1128C, N1128R, N1128S, N1128T, N1128W, A1129E, A1129L, A1129N, A1129Q, A1129R, A1129V, V1130A, V1130G, V1130P, V1130R, V1130S, R1131A, R1131N, R1131Q, R1131S, R1131W, F1132E, F1132K, F1132M, F1132P, F1132Q, F1132T, P1133D, P1133G, P1133L, P1133Q, P1133R, P1133V, D1134E, D1134G, D1134L, A1135E, A1135K, A1135L, A1135M, A1135S, A1135W, A1135Y, G1136A, G1136E, G1136P, G1136Q, G1136T, K1137A, K1137C, K1137G, K1137L, K1137P, K1137Q, K1137R, K1137S, K1137T, K1137V, T1138R, T1138Y, K1139A, K1139L, K1139R, K1139T, I1140A, I1140C, I1140G, I1140L, I1140M, I1140P, I1140R, I1140T, Q1141A, Q1141C, Q1141G, Q1141K, Q1141N, Q1141P, Q1141T, Q1141W, I1142E, I1142R, I1142S, I1142W, I1142Y, S1143G, A1144C, A1144D, A1144E, A1144N, A1144P, A1144S, A1144T, A1144V, A1144W, D1145E, D1145R, D1145T, G1146A, G1146C, G1146D, G1146K, G1146L, G1146R, G1146V, K1147A, K1147G, K1147T, K1147V, N1148H, N1148I, N1148K, N1148P, N1148Q, N1148R, N1148S, N1148T, N1148W, W1149C, W1149G, W1149I, W1149K, W1149N, W1149Q, W1149S, W1149T, W1149V, W1149Y, T1150G, T1150K, T1150P, D1151C, D1151G, D1151R, D1151T, D1151W, L1152A, L1152C, L1152E, L1152Q, L1152W, A1153E, A1153G, A1153K, A1153L, A1154C, A1154D, A1154E, A1154G, A1154R, A1154S, T1155E, T1155L, T1155Q, T1155R, T1157V, T1157W, I1158R, I1158S, I1158W, A1159C, A1159E, A1159I, A1159P, A1159R, A1159V, A1160K, A1160L, A1160Q, A1160S, Q1161A, Q1161P, Q1161S, E1162A, E1162C, E1162D, E1162F, E11621, E1162N, E1162Q, E1162T, E1162W, E1162Y, E1165H, E1165L, E1165M, E1165R, E1165S, E1165W, R1166D, R1166K, R1166Q, V1167A, V1167C, V1167L, V1167P, V1167R, K1168L, K1168Q, K1168R, K1168W, P1169M, P1169R, P1169S, Y1170E, Y1170K, Y1170M, Y1170Q, Y1170R, Y1170V, T1171A, T1171G, T1171M, T1171Q, T1171R, T1171S, Y1172D, Y1172E, Y1172H, Y1172I, Y1172K, Y1172L, Y1172S, Y1172V, D1173A, D1173E, D1173F, D1173G, D1173K, D1173L, D1173P, D1173R, D1173T, D1173W, F1174D, F1174P, F1174Q, F1174R, F1174S, F1174T, F1174V, F1174W, A1175G, A1175I, A1175N, A1175Q, A1175S, A1175V, A1175Y, V1177N, V1177P, V1177S, V1177T, G1178M, G1178Q, G1178S, G1178T, A1179L, A1179P, A1179Q, A1179W, T1180A, T1180G, T1180I, T1180L, T1180M, T1180Q, T1180S, T1180Y, F1181E, F1181L, F1181V, V1182M, K1183A, K1183E, K1183T, K1183V, V1184C, V1184E, V1184K, V1184L, V1184P, V1184Q, V1184Y, T1185Q, T1185V, V1186S, N1188V, N1188W, A1189C, A1189K, A1189T, A1189V, D1190R, D1190S, D1190T, D1190Y, T1191E, T1191L, T1192H, T1192P, T1193G, P1194A, P1194E, P1194G, P1194W, S1195G, V1197A, V1198E, C1199D, C1199T, A1200G, A1200W, L1202A, L1202C, T1203E, T1203K, E1204G, E1204S, I1205V, K1208R, K1208V, K1208W, T1209A, T1209C, T1209E, T1209K, T1209N, T1209Q, T1209R, T1209W, A1210D, A1210E, A1210G, A1210K, A1210L, A1210Q, A1210R, A1210T, A1210W, T1211C, T1211D, T1211E, T1211G, T1211H, T1211K, T1211P, T1211Q, T1211R, T1211S, T1211V, K1213A, K1213D, K1213S, K1213T, K1213W, F1214A, F1214E, F1214K, F1214L, F1214P, F1214R, F1214S, F1214V, V1215D, V1215E, V1215K, V1215L, V1215Q, V1215S, V1215W, T1216L, T1216P, T1216Q, T1216R, N1217A, N1217D, N1217E, N1217F, N1217P, N1217R, N1217S, N1217T, T1218A, T1218C, T1218E, T1218G, T1218Q, T1218S, T1218V, T1218W, S1219A, S1219E, S1219F, S1219I, S1219K, S1219R, S1219V, A1220C, A1220G, A1220L, A1220P, A1220R, A1220V, A1221D, A1221G, A1221K, A1221L, A1221R, A1221V, A1221W, L1222A, L1222C, L1222E, L1222F, L1222Q, L1222R, L1222V, L1222W, S1223C, S1223F, S1223G, S1223K, S1223L, S1223V, S1224A, S1224D, S1224G, S1224L, S1224M, S1224P, S1224R, S1224W, L1225C, L1225D, L1225E, L1225F, L1225G, L1225K, L1225P, L1225T, L1225V, L1225W, T1226A, T1226G, T1226M, T1226P, T1226R, T1226S, T1226V, T1226Y, V1227A, V1227C, V1227D, V1227E, V1227G, V1227L, V1227P, V1227Q, V1227S, N1228A, N1228D, N1228F, N1228K, N1228L, N1228T, G1229A, G1229C, G1229E, G1229Q, G1229S, G1229V, T1230F, T1230H, T1230I, T1230K, T1230L, T1230P, T1230R, T1230S, T1230W, K1231F, K1231G, K1231L, K1231M, K1231P, K1231S, K1231W, V1232E, V1232K, V1232Q, V1232R, V1232S, V1232T, V1232W, S1233P, S1233W, D1234G, D1234K, D1234R, D1234V, S1235D, S1235E, S1235G, S1235L, S1235P, S1235R, S1235W, S1235Y, V1236A, V1236C, V1236G, V1236I, V1236P, V1236Q, V1236R, L1237D, L1237E, L1237R, L1237V, L1237W, A1238D, A1238E, A1238K, A1238L, A1238N, A1238P, A1238R, A1238S, A1238T, A1239D, A1239P, A1239R, G1240D, G1240L, G1240N, G1240Q, G1240S, G1240T, G1240W, S1241D, S1241G, S1241I, S1241L, S1241M, S1241P, Y1242C, Y1242E, Y1242K, Y1242R, Y1242S, Y1242W, N1243C, N1243L, N1243M, N1243P, N1243Q, N1243S, N1243T, N1243V, N1243W, T1244A, T1244D, T1244E, T1244G, T1244L, T1244Q, T1244S, T1244V, T1244W, A1246F, A1246M, A1246N, A1246P, A1246Q, A1246R, A1246S, A1246T, I1247A, I1247G, I1247M, I1247Q, I1247R, I1247S, I1247T, I1247V, I1247W, I1248A, I1248G, I1248K, I1248L, I1248R, I1248S, I1248Y, A1249E, A1249G, A1249H, A1249I, A1249R, A1249T, A1249V, D1250I, D1250K, D1250S, D1250T, D1250W, D1250Y, V1251I, V1251T, V1251W, K1252D, K1252G, K1252V, K1252W, A1253P, A1253V, E1254F, E1254G, E1254H, E1254L, E1254R, E1254V, G1255H, G1255M, G1255S, G1255V, E1256G, E1256M, E1256N, E1256R, E1256V, E1256W, G1257F, G1257K, G1257L, G1257Q, G1257R, G1257W, N1258C, N1258G, N1258H, N1258K, N1258S, A1259K, A1259L, A1259W, V1261I, V1261L, V1261P, V1261Q, V1261R, V1261T, T1262A, T1262F, T1262M, T1262Q, T1262R, V1263E, V1263G, V1263Q, V1263R, V1263T, V1263W, L1264A, L1264E, L1264H, L1264R, L1264S, L1264Y, P1265C, P1265K, P1265L, P1265R, P1265S, P1265V, P1265W, A1266F, A1266L, A1266P, A1266S, A1266V, H1267A, H1267E, H1267F, N1269A, N1269E, N1269K, N1269R, N1269S, N1269T, N1269W, V1270D, V1270E, V1270G, V1270I, V1270L, V1270T, V1270W, I1271A, I1271H, I1271Q, R1272E, R1272F, R1272M, R1272P, R1272V, V1273R, I1274F, I1274M, I1274R, T1275A, T1275L, T1275W, E1276R, E1276W, S1277L, S1277T, S1277W, E1278Q, E1278R, D1279G, D1279I, D1279R, D1279T, D1279V, D1279W, H1280C, H1280E, H1280G, H1280V, H1280W, V1281F, V1281I, V1281S, V1281W, T1282D, T1282L, T1282V, R1283A, R1283D, R1283E, R1283P, R1283W, K1284G, T1285A, T1285E, T1285F, T1285G, T1285M, T1285R, T1285Y, F1286A, F1286E, F1286P, F1286R, F1286S, F1286T, T1287C, T1287K, T1287L, T1287M, T1287Q, T1287R, T1287S, T1287W, I1288A, I1288D, I1288F, I1288G, I1288K, N1289A, N1289Q, N1289T, L1290A, L1290G, L1290R, L1290V, L1290W, G1291K, G1291P, G1291V, G1291W, G1291Y, T1292G, T1292L, T1292Y, E1293G, E1293K, E1293L, E1293S, E1293V, Q1294E, Q1294L, Q1294P, Q1294W, E1295K, F1296A, F1296G, F1296I, P1297F, A1298Y, D1301I, E1302G, E1302R, E1302S, R1303S, D1304A or D1304V.

77. The lactase variant of the preceding embodiment, wherein the variant has a decreased or an increased galactose inhibition, preferably measured as a galactose inhibition improvement factor (GI-IF) of at least 1.1 or at most 0.9, compared to the lactase of SEQ ID NO: 1.

78. The lactase variant of embodiment 1 which comprises one or more of the following substitutions, wherein each position corresponds to a position in SEQ ID NO: 1: V1D, V1F, V1H, V1K, V1L, V1R, E2D, E2G, E2Q, E2V, D3A, D3I, D3N, D3V, A4H, T5A, T5S, R6G, R6H, R6L, R6W, S7L, S7N, S7P, S7T, S9G, S9H, S9W, T10K, T10L, T10S, Q12V, M13C, M13D, M13E, M13H, M13K, M13R, M13W, S14G, S14H, S14T, S14V, S14Y, S15C, S15F, S15I, S15K, S15L, S15P, S15R, S15T, S15V, S15W, T16C, T16I, P17A, P17I, P17S, V19A, V19F, V19I, V19K, V19L, V19N, V19S, V20C, V20F, V20G, V20I, V20L, V20M, V20P, V20Q, V20R, V20T, Y21A, Y21C, Y21D, Y21G, Y21H, Y21M, Y21P, Y21R, Y21T, S22A, S22N, S22R, S22T, S22W, S23A, A24F, A24L, A24R, A24T, A24W, V25D, V25E, V25F, V25G, V25M, V25Q, V25S, V25T, V25W, K28L, K28R, K28S, K28V, K28W, Q29F, Q29G, Q29L, Q29M, Q29R, Q29S, N30M, N30V, N30W, N30Y, R31E, R31M, R31V, T32Q, T32S, S33K, D34F, D34G, D34H, F35A, F35C, F35G, F35V, D36Q, A37N, N38S, W39G, W39S, K40C, K40F, K40G, K40I, K40M, K40P, K40W, F41A, F41C, M42T, L43A, L43G, L43I, L43S, S44M, D45L, D45P, D45V, V47K, V47R, A49C, A49D, A49H, A49T, D51G, D51I, D51K, D51M, A53G, D55C, D55F, D55G, D55H, D55M, D55N, D55P, D55S, D55V, S57A, S57C, S57E, S57G, A58I, A58N, A58Q, A58R, A58T, W59D, W59I, W59K, W59L, W59N, W59P, W59V, Q60E, Q60F, Q60G, Q60L, Q60M, Q60R, Q60S, Q60V, Q60Y, Q61P, V62G, V62N, V62T, V62W, D63G, D63L, D63S, L64E, L64G, H66R, H66T, I70H, I70K, I70P, 170R, T71H, T71L, T71P, T71R, T71S, K73A, K73D, K73G, K73Q, K73V, Y74G, Y74K, S75G, S75L, S75Q, S75R, S75V, Q76G, Q76I, Q76P, S77C, S77D, S77E, S77G, S77H, S77I, S77K, S77L, S77M, S77R, S77T, S77V, S77W, N78C, N78F, N78S, E79H, E79S, E79T, A83E, A83T, L85A, L85C, L85D, L85F, L85N, L85S, L85V, L85W, P86E, P86G, P86Q, P86R, P86V, P86W, P86Y, G87D, G87N, G88A, G88F, G88M, T89C, T89L, T89M, T89N, T89W, T89Y, G90A, G90D, G90L, G90S, G90T, W91E, W91L, W91P, W91Q, W91Y, Y92F, Y92H, Y92I, Y92M, Y92N, Y92S, Y92T, Y92V, Y92W, R93A, R93D, R93F, R93H, R93I, R93L, R93N, R93T, R93V, R93Y, K94A, K94C, K94G, K94R, K94S, K94T, K94V, S95A, S95C, S95D, S95E, S95G, S95I, S95L, S95Q, S95R, F96A, F96C, F96I, F96K, F96L, F96M, F96P, F96V, F96W, T97F, T97S, T97V, I98C, I98H, I98S, I98W, R100T, D101A, D101P, D101V, L102A, L102G, L102M, L102P, L102S, G104C, K105D, K105Q, K105R, K105W, K105Y, R106K, R106V, R106W, I107A, I107F, I107S, A108E, A108V, I109M, I109T, N110A, N110F, N110S, N110T, N110V, N110W, F111A, F111C, F111L, F111Q, F111V, D112A, V114F, V114R, Y115E, M116A, M116C, M116D, M116W, N117K, A118P, T119G, V120A, V120K, W121C, W121D, W121R, W121T, W121V, G124M, V125D, K126E, K126V, T129V, H130A, H130C, H130S, H130T, P131K, Y132E, F137C, F137D, F137L, F137P, S138A, S138D, S138H, S138M, D140V, L141G, T142S, G148K, G149E, G149I, G149M, G149Y, E150A, E150C, E150G, E150L, E150N, E150R, N151L, V154E, V154I, V154K, V154L, V154M, V157A, V157G, V157P, V157Q, V157S, E158H, E158K, E158Q, E158V, N159D, N159H, N159T, R160G, L161E, L161K, L161M, L161S, L161W, P162F, P162G, P162N, P162T, P162W, S164A, R165H, W166S, Y167A, Y167C, G169A, G169S, S170Q, G171C, G171F, I172G, I172K, I172P, I172Q, Y173A, Y173H, Y173M, Y173P, Y173S, R174E, R174K, D175Y, V176E, V176K, V176T, T177C, T177E, T177K, L178I, L178Q, L178W, T179A, T179C, T179D, T179H, T179I, T179K, T179L, T179N, T179P, T179S, V180A, V180C, V180D, V180E, V180G, V180M, T181A, T181F, G183W, V184F, V184H, V184P, V184Q, V184R, V184S, V184W, H185G, H185L, H185R, V186A, V186E, V186G, V186N, G187A, G187D, G187H, N188R, N188S, N188V, N188W, N189A, G190F, G190V, V191T, V191Y, I193N, I193Q, I193T, I193V, K194I, T195M, T195W, P196A, P196I, P196M, P196S, P196W, S197C, S197E, S197L, L198I, A199E, A199P, A199T, Q201E, Q201K, N202A, N202M, N202Q, N202S, N202W, G203Q, G204R, N205W, V206C, T207L, K213A, A215D, N216D, D217F, D217G, D217L, D217M, T218D, T218G, T218H, K219A, A220G, A221C, A221D, A222R, N223S, I224G, T225L, L226Q, Q228N, Q228R, F231L, P232T, G234V, G235K, K236M, K236P, T237D, D238K, A240Q, G242K, G242L, G242M, G242P, T243I, A247K, S248F, I251F, I251W, I251Y, A252H, A252P, A252W, A252Y, A257D, A257G, A257N, A257V, T260V, S261A, S261H, S261Y, T262E, T262F, T262P, I263A, T264L, A266D, S267V, P268F, P268R, L270N, W271T, S272T, S272W, I273W, K274Q, N275M, N277F, T280D, V281Q, R282W, T283M, V285H, L286W, V291H, V291L, V291P, V291S, L292A, L292D, L292E, L292Q, L292S, L292V, D293C, D293I, D293W, T294C, T294M, T294Q, T294S, Y295F, Y295G, Y295L, Y295S, D296F, D296H, D296K, T297I, T297S, E298I, E298L, E298M, E298R, E298T, Y299F, Y299S, F301C, R302I, R302K, R302M, R302T, W303A, W303C, W303D, W303F, W303T, T304D, T304E, T304I, T304K, T304P, T304S, T304W, G305E, G305L, G305M, G305N, G305P, G305T, D307A, D307E, D307F, D307S, D307V, D307W, A308G, A308I, T309C, T309D, T309E, T309I, T309K, T309V, S310A, S310C, S310G, S310H, S310I, S310L, S310M, S310N, S310R, S310V, G311F, G311L, F312H, F312L, F312V, L314A, L314V, L314Y, N315S, G316N, G316R, G316W, G316Y, E317V, E317W, E317Y, K318C, K318E, K318F, K318H, K318M, K318N, K318P, K318Q, K320E, K320I, K320V, K320W, L321H, L321M, L321Y, K322C, K322I, V324E, V324G, V324T, S325G, M326T, M326V, H328C, H328D, H328F, H328G, H3281, H328L, H328M, H328R, H328T, G331V, A335C, A335G, A335L, A337D, A337E, A337G, A337L, N338P, N338R, R340A, R340L, A341M, I342A, I342G, I342P, E343A, E343N, E343R, E343T, E343Y, R344G, R344Y, Q345F, Q345N, Q345S, V346A, V346I, V346S, E347A, E347D, E347I, I348A, I348D, I348G, I348M, I348Q, I348R, L349A, L349E, L349S, L349V, Q350G, Q350N, Q350W, K351R, K351W, M352L, M352T, G353K, V354E, V354G, V354S, V354W, N355H, I357T, T359E, T359L, T360V, N362S, N362T, P363A, P363D, P363I, P363L, P363M, P363S, P363V, A364C, A364E, A364F, A364G, A364I, A364M, A364P, A364V, A364W, A365C, A365I, A365P, A365V, A365W, K366D, K366I, K366L, K366M, K366P, K366S, K366V, A367I, A367N, A367Q, L368A, L368Q, L368S, L368V, L368Y, I369A, I369D, I369E, I369G, I369K, I369M, I369V, I369W, D370L, D370Q, D370R, D370T, V371F, V371G, V371I, V371L, V371Q, C372P, N373G, N373L, N373R, E374L, K375D, K375I, K375N, K375Q, K375S, G376A, G376S, V377A, V377M, V377T, L378I, L378P, L378Y, V379A, V379C, V379M, V379N, V380P, V380S, E381A, E381C, E381G, E381Q, E381T, V383A, M386G, M386N, M386S, M386V, W387H, N388E, N388L, R389A, R389C, R389E, R389K, R389M, R389Q, R389S, R389T, R389V, S390C, S390D, S390H, S390P, S390T, G393E, G393R, G393V, T395A, T395C, T395F, E396K, E396L, E396V, E396W, Y398M, G399S, K400A, K400C, K400M, K400N, K400T, K400V, W401F, W401K, F402W, F402Y, G403A, G403D, G403H, G403K, G403Q, G403T, G403V, G403Y, Q404F, Q404L, Q404M, Q404P, Q404S, Q404V, A405C, A405E, A405K, A405R, A405T, A405V, I406C, I406D, I406N, A407C, A407G, A407Q, N410R, N410Y, A411E, A411R, A411S, V412S, G414M, G414N, G414T, G415A, G415Q, D416M, K417F, K417R, A422P, K423L, K423R, L426C, T427D, T427F, T427Q, T427S, T427W, T429D, T429P, I430L, I430M, N431L, N431M, R432F, R432G, R432Q, R432Y, D433C, D433G, D433I, D433P, D433Q, D433W, R434N, R434T, R434V, N435E, N435F, N435H, N435L, N435V, A436C, A436G, A436L, A436M, P437K, P437L, P437W, S438G, V439C, V439K, I440S, I440T, I440V, M441R, M441T, W442E, W442G, S443C, S443G, S443M, S443Q, L444E, L444F, L444V, N446D, M448C, M448E, M448L, M449D, M449E, M449F, M449T, M449V, M449W, M449Y, E450F, E450S, E450T, E450V, E450W, G451C, G451F, G451L, G451P, G451V, G451W, I452K, I452L, I452Q, I452S, I452V, S453C, S453G, S453H, S453L, S453M, S453N, S453P, S453Q, S453R, S453V, G454L, G454W, S455E, S455M, S455P, S455R, S455V, V456D, V456E, V456F, V456K, S457H, S457Q, S457T, S457V, G458A, G458D, G458P, G458S, F459C, F459R, P460M, P460Y, A461G, A461M, A461S, T462E, T462L, A464W, K465C, K465G, K465L, K465Q, K465V, K465Y, L466G, L466P, V467T, A468D, W469L, W469R, K471F, K471Q, K471Y, A472G, A472Y, A473M, A473P, D474C, D474E, D474M, D474W, S475E, S475T, T476L, T476S, R477A, R477G, R477L, P478A, P478D, P478V, M479G, T480Q, K485E, K485R, K487A, K487C, K487F, K487S, K487W, A488C, A488L, A488V, S493E, S493G, S493H, S493L, S493Q, N494A, N494I, N494M, N494R, N494V, T495K, G497D, N499T, G505D, G505H, G505L, G505R, G505S, G505V, V506C, V506E, V506R, V507F, V507P, V507R, G508C, G508E, T509A, T509D, T509E, T509I, T509K, T509M, T509Q, T509S, T509V, T509Y, N510A, N510F, N510I, Y511A, S512C, S512E, S512F, S512I, S512M, S512Q, S512T, S512V, S512Y, D513G, D513P, D513R, A515E, A515F, A515G, A515P, A515S, N516C, N516E, N516G, N516I, N516M, N516Q, N516S, N516T, N516V, Y517G, Y517I, D518Y, K519C, K519E, K519F, K519G, K519I, K519L, K519M, K519N, K519Q, K519S, K519T, I520A, I520C, I520G, I520H, I520M, I520N, I520Q, I520S, I520T, I520W, I520Y, R521A, R521C, R521K, R521Q, R521V, T523A, T523M, H524R, H524V, P525R, S526G, W527A, W527E, W527G, W527H, W527N, W527R, W527S, A528E, A528G, A528L, I529G, A535E, A535G, A535I, A537M, A537P, A537S, I538H, I538M, S540E, N545G, T547K, G549D, G549P, G549W, G550Q, G550R, S553P, S553R, D555E, D555P, K556C, K556R, L558E, L558H, L558P, T559G, T559Q, T559V, T559Y, S560P, S560V, S564A, S564C, S564F, S564W, A565C, A565D, A565E, A565F, A565G, A565I, A565L, A565M, A565R, A565S, A565T, A565V, A565W, G567N, G567Q, G567V, A570G, A570L, A572W, S574V, A575D, Y577R, D578T, V580D, Q581T, Q581Y, R582A, R582G, R582I, R582L, D583V, D583W, F584I, F584W, A586D, T588P, Y589I, Y589Q, W591F, T592L, G593C, F594C, L597D, L597T, G598N, P600A, P600E, P600G, P600S, N604E, N604S, G605R, G607Q, G607S, S608E, S608I, S608N, S608Q, S608T, G609A, G609S, A610T, G612V, S613A, W614A, P615V, N619I, N619L, N619S, Y621W, V625A, V625E, V625F, V625M, V625Y, T627K, T627Q, A628C, A628D, A628N, G629T, F630C, F630Y, P631D, P631Y, K632C, K632D, K632G, D633G, T634F, T634S, T634V, Y636K, F637C, F637G, F637I, F637S, F637T, F637V, Q639R, N643R, D644Y, D645S, D645V, V646C, V646L, V646N, V646R, V646S, H647G, H647V, T648C, L649V, H650F, I651T, I651V, L652C, L652D, L652V, L652W, P653Q, A654D, A654R, W655F, W655R, N656K, E657K, E657R, E657V, A661E, A661G, A661H, A661K, A661L, A661M, A661Q, A661W, K662H, K662S, K662V, K662W, K662Y, N667L, N667R, P669A, P669E, P669L, P669R, P669T, P669W, V672L, Y673S, T674D, T674M, T674Q, D675A, D675V, D675Y, A676L, A676P, A677T, V679Q, V679S, K680I, K680N, Y682V, F683W, T684R, K686E, G687P, S688K, S688L, T689G, T689W, E690D, K691E, K691H, K691P, K691R, R692G, R692H, R692I, R692L, R692P, R692S, R692T, R692V, R692W, L693M, L693P, I694L, I694W, G695C, G695K, G695L, G695R, G695W, E696R, K697A, K697G, K697R, K697V, K697W, S698D, S698E, S698M, S698Q, S698R, T700A, T700D, T700Y, K701A, K701D, K701G, K701H, K701L, K701M, K701P, K701S, T703K, A705D, A705K, A705P, A705R, A705W, A706C, A706T, A706W, Y708F, Y708L, Y710E, Y710T, Y710V, Q711A, Q711D, Q711T, V712M, V712P, V712Q, Y713E, Y713G, E714H, E7141, E714K, E714N, G715K, A716M, A716P, A716R, A716T, D717G, D717L, D717S, K718A, K718Q, K718T, K718W, K718Y, D719E, D719S, D719T, D719V, S720A, S720M, S720R, S720Y, T721D, T721H, T721K, T721L, T721N, T721P, T721Q, T721S, T721V, T721W, T721Y, A722Q, A722T, A722V, H723G, H723P, K724A, K724G, N725A, N725I, M726D, M726Q, Y727L, L728C, L728K, L728S, L728W, T729A, T729E, T729M, N731E, N731Q, V732R, P733W, W734G, W734R, A735Q, A735R, A735S, A735T, G737F, G737K, G737N, G737Y, T738L, T738S, I739E, I739K, S740D, S740F, A741I, A741P, A741S, A741V, E742V, A743E, A743I, Y744L, Y744R, D745C, D745N, D745R, E746C, E746K, E746T, N747E, N747F, N747P, R749M, L750M, L750P, L750Q, L750S, I751C, I751H, P752C, P752Y, G754H, G754I, G754R, S755T, T756N, T756Q, E757A, G758V, N759S, N759V, A760G, S761A, V762W, T765P, G766M, A769F, A769G, A769I, A769N, A769V, A769Y, K770H, L771A, K772A, K772C, K772P, K772W, A773C, A773M, A773R, D774A, A775V, D776R, D776V, R777H, R777S, R777T, K778F, K778G, K778N, K778R, T779I, T781A, T781E, T781F, T781G, T781P, T781Y, A782C, A782N, A782Q, A782Y, D783A, K785C, K785I, K785S, K785V, K785W, K785Y, D786V, L787D, L787K, Y789V, I790R, I790V, E791D, E791F, E791S, E791V, E791W, V792S, V792Y, D793C, D793Y, V794D, D796S, D796T, A797H, N798I, N798Q, G799D, G799K, G799L, G799Y, H800F, H800G, H800V, I801W, V802E, V802I, V802S, V802Y, P803G, P803S, P803Y, D804E, A805C, A805F, A805I, A805N, A805P, A806F, A806I, A806Q, N807F, R808F, R808G, R808I, R808P, V809L, V809M, V809P, T810L, T810P, T810Q, T810R, T810Y, F811L, V813H, V813W, V813Y, K814G, K814P, G815F, G815M, G815P, A816D, A816N, A816W, G817H, K818D, K818F, K818L, K818Q, K818R, K818S, K818W, K818Y, L819F, V820C, V820R, V820W, G821I, V822A, V822D, V822E, V822T, D823E, N824A, G825A, S826A, S826G, S826I, S826W, P828G, D829C, D829I, D829S, H830Q, D831A, D831M, D831P, S832P, S832R, S832W, Y833C, Y833D, Y833E, Y833N, Q834M, A835D, A835H, A835K, D836C, D836E, D836Q, D836R, D836S, D836T, D836V, D836Y, R838N, R838S, K839A, K839D, K839L, K839N, A840G, A840I, A840P, A840V, F841I, F841K, S842A, K844A, K844Y, A847T, I848M, Q850L, S851C, S851D, S851E, S851T, T852L, K853P, E854C, E854I, A855K, A855V, A855Y, I858D, I858G, I858K, I858P, V860Y, T861F, T861I, T861Q, T861V, T861W, A862C, A864K, D865A, D865G, G866H, G866R, L867W, S870E, S870M, S870T, V872C, V872G, K873G, K873Y, I874G, A875R, V879S, T882A, T882M, T882R, S883L, E885V, K886L, K886V, K886W, T887A, T887D, T887G, T887N, V888A, R889G, Y892P, Y893E, Y893G, S894D, S894G, Y897V, Y898T, K900E, K900G, T901Q, T901R, T901Y, G902A, G902D, G902F, G902P, G902Q, G902R, G902S, G902W, N903D, N903I, K904D, K904E, K904M, K904N, K904R, K904S, K904V, K904W, P905A, P905C, P905R, P905V, P905W, P905Y, I906A, I906N, I906T, I906W, L907F, L907S, P908C, P908D, P908G, P908I, P908L, P908T, S909E, S909F, S909G, S909W, S909Y, D910I, D910W, V911A, E912A, E912K, E912L, E912T, E912V, V913G, V913R, R914A, R914E, R914F, R914I, R914K, R914V, R914W, R914Y, Y915A, Y915C, Y915G, Y915I, Y915M, Y915Q, Y915V, S916G, D917C, D917E, D917F, D917L, D917M, D917Q, D917S, G918E, G918H, G918T, G918V, T919D, T919K, T919Q, T919W, S920P, S920V, D921C, D921V, R922A, R922M, R922V, R922W, Q923A, Q923E, Q923L, Q923M, Q923V, N924Q, N924S, V925C, V925E, V925G, V925K, V925N, V925W, T926P, T926R, W927P, D928A, D928H, D928Q, A929P, V930E, V930I, V930T, S931P, S931R, D933R, Q934S, Q934V, I935A, I935C, I935D, I935E, I935L, I935P, I935V, I935W, A936I, A936L, A936Q, A936R, A936Y, K937G, K937I, K937M, K937P, K937Q, A938C, A938H, A938N, A938T, A938V, A938W, G939D, G939K, S940C, S940E, S940R, S940V, F941C, F941M, S942A, S942E, S942K, S942P, S942V, V943A, V943G, V943Q, T946A, T946L, T946P, T946W, V947G, V947L, V947P, V947R, V947T, A948I, A948W, G949A, G949V, Q950K, Q950W, K951D, K951G, K951P, K951S, K951W, K951Y, I952Q, S953F, S953M, S953N, S953R, V954D, V954Q, R955A, R955C, R955K, R955Q, R955W, V956A, V956D, V956G, V956H, V956I, V956M, V956Q, V956W, T957D, T957S, T957W, I959A, I959L, I959S, I959V, I959Y, D960P, D960S, E961D, E961F, E961K, E961S, E961T, I962A, I962C, I962D, I962G, I962K, I962N, G963C, G963E, G963P, L965C, L965E, L965G, L965K, L965M, L965P, L965Q, L965S, L965V, L965Y, L966A, L966K, L966N, L966Q, L966S, L966V, N967D, N967I, N967L, N967M, N967P, N967T, N967V, Y968G, Y968L, Y968Q, Y968V, S969C, S969D, S969G, S969H, S969I, S969L, S969M, S969P, S969Q, S969Y, A970I, A970L, S971G, S971V, S971W, P973C, P973D, P973K, P973N, P973Q, P973R, P973V, P973W, P973Y, V974C, V974E, V974G, V974N, V974T, V974Y, G975F, G975K, T976D, T976F, T976G, T976K, T976L, T976P, T976S, P977C, P977K, P977R, P977T, P977Y, A978F, A978G, A978M, A978N, A978R, A978S, A978Y, V979G, V979N, V979Y, L980A, L980F, L980H, L980I, L980K, L980N, L980Q, L980T, L980Y, P981L, P981M, R984P, P985F, P985H, P985K, P985L, P985W, A986C, A986I, A986L, A986M, A986W, V987A, V987C, V987F, V987K, L988V, P989M, P989W, D990F, D990P, D990W, G991C, G991F, G991H, G991K, G991P, G991Y, T992M, T992N, V993G, S995E, S995L, S995R, A996R, N997K, N997V, F998M, F998W, V1000W, D1001L, D1001S, W1002A, W1002D, W1002E, W1002H, W1002N, W1002P, W1002Q, W1002S, T1003F, T1003G, T1003N, T1003R, T1003S, T1003W, T1003Y, K1004D, K1004E, K1004S, P1005I, P1005N, P1005V, P1005Y, A1006C, A1006N, A1006P, A1006S, A1006V, A1006W, A1006Y, D1007C, D1007L, D1007P, D1007V, T1008G, Y1010A, Y1010P, Y1010R, Y1010T, N1011A, N1011S, N1011T, N1011W, T1012H, T1012I, T1012Q, A1013D, A1013Q, A1013V, G1014I, G1014M, G1014V, G1014W, T1015A, V1016D, V1016P, K1017E, K1017G, V1018K, V1018L, V1018R, V1018S, T1021C, T1021E, T1021G, A1022H, V1024G, V1024H, V1024K, V1024R, V1024S, V1024W, G1026E, G1026S, G1026V, G1026Y, K1027Q, K1027R, E1028G, E1028T, F1029I, F1029K, F1029P, F1029V, K1030D, K1030H, K1030M, K1030W, V1031H, V1031K, A1033V, T1034G, T1034H, T1034N, I1035D, I1035G, I1035Q, R1036G, R1036L, R1036T, R1036Y, V1037C, Q1038D, R1039V, S1040M, Q1041P, V1042N, T1043F, T1043G, T1043R, I1044A, I1044L, S1046M, V1048C, V1048F, V1048G, V1048I, V1048M, V1048Q, N1051A, N1051E, N1051K, A1052C, A1052K, A1052R, L1053A, L1053W, R1054C, R1054L, L1055R, L1055T, Q1057A, Q1057E, Q1057P, N1058R, N1058S, P1060G, P1060N, P1060Q, A1061G, A1061K, A1061W, D1062A, D1062G, D1062I, D1062M, K1063M, Q1064M, Q1064R, Q1064T, Q1064V, S1065A, S1065C, D1066A, D1066G, D1066V, T1067G, T1067M, L1068P, L1068Q, L1068Y, D1069R, A1070T, I1071M, K1072P, K1072S, D1073F, D1073W, G1074L, G1074R, S1075G, S1075L, T1076E, T1076S, T1077R, V1078D, V1078W, D1079G, D1079L, N1081E, T1082E, T1082F, T1082G, T1082K, G1083E, G1083F, G1083L, G1084M, G1084V, G1084W, G1084Y, G1085P, G1085R, G1085S, A1086H, A1086K, A1086R, N1087A, N1087E, N1087I, N1087R, P1088D, P1088E, P1088G, P1088R, P1088W, S1089G, S1089K, S1089Q, S1089V, A1090F, W1091Y, N1093A, N1093G, N1093L, N1093P, N1093Q, W1094D, W1094E, W1094P, W1094R, W1094T, A1095P, A1095T, A1095W, Y1096D, Y1096L, S1097E, S1097K, S1097T, K1098D, K1098F, K1098G, K1098Q, K1098S, A1099D, A1099S, A1099V, A1099W, G1100E, G1100H, G1100N, H1101K, H1101Q, H1101R, H1101V, N1102H, N1102Q, N1102T, T1103A, T1103E, T1103S, T1103W, E1105L, E1105S, E1109A, E1109D, E1109L, E1109W, A1111G, A1111S, E1113G, E1113P, E1113V, Q1114E, Q1114L, Q1114R, Q1114S, Q1114T, Q1114V, Q1115A, Q1115K, Q1115L, Q1115P, L1116D, L1116G, L1116H, L1116K, L1116W, G1117E, G1117I, G1117M, G1117R, G1117S, G1117T, G1117V, G1117W, Q1118M, Q1118W, I1119D, I1119E, I1119N, I1119S, V1120N, V1120S, M1121G, M1121K, M1121N, M1121P, M1121S, F1123E, F1123R, F1124V, F1124W, R1125D, R1125F, R1125K, R1125V, R1125W, S1127I, S1127Q, N1128A, A1129E, A1129R, V1130A, V1130R, R1131W, F1132M, F1132P, F1132Q, P1133D, P1133G, P1133L, P1133R, A1135E, A1135K, A1135L, A1135W, G1136P, K1137G, K1137P, K1137R, K1137S, K1137T, K1137V, T1138R, T1138Y, I1140C, I1140P, I1140R, Q1141A, Q1141G, Q1141K, Q1141N, Q1141T, I1142E, I1142R, I1142Y, S1143G, A1144D, A1144N, A1144P, A1144S, A1144T, A1144V, A1144W, D1145E, D1145R, D1145T, G1146A, G1146K, G1146R, G1146V, N1148H, N1148I, N1148P, N1148Q, N1148R, N1148S, N1148T, W1149C, W1149G, W1149K, W1149S, W1149V, T1150K, T1150P, D1151C, D1151W, L1152A, L1152C, L1152E, L1152Q, L1152W, A1153E, A1153G, A1153L, A1154D, A1154E, A1154R, I1158S, I1158W, A1159E, A1159I, Q1161P, Q1161S, E1162A, E1162C, E1162Q, E1162W, E1165M, E1165S, R1166Q, V1167A, V1167R, K1168L, K1168R, P1169M, P1169S, Y1170K, Y1170M, Y1170V, Y1172H, Y1172S, D1173G, D1173T, F1174D, A1175S, G1178M, G1178Q, G1178S, G1178T, A1179L, A1179Q, A1179W, T1180A, T1180G, T1180I, T1180L, T1180M, T1180Q, T1180S, T1180Y, F1181E, F1181V, V1182M, K1183A, K1183E, K1183T, K1183V, V1184C, V1184E, V1184K, V1184L, V1184P, V1184Q, V1184Y, T1185Q, T1185V, V1186S, N1188V, N1188W, A1189C, A1189K, A1189T, A1189V, D1190R, D1190T, D1190Y, T1191L, T1193G, P1194E, P1194W, V1197A, C1199D, A1200G, T1203K, E1204G, E1204S, I1205V, K1208R, K1208V, K1208W, T1209A, T1209C, T1209E, T1209K, T1209N, T1209Q, T1209R, T1209W, A1210D, A1210G, A1210K, A1210L, A1210Q, A1210R, A1210T, A1210W, T1211C, T1211D, T1211E, T1211G, T1211H, T1211K, T1211P, T1211Q, T1211R, T1211S, T1211V, K1213A, K1213D, K1213S, K1213T, F1214A, F1214E, F1214K, F1214L, F1214P, F1214S, F1214V, V1215D, V1215E, V1215L, V1215Q, V1215W, T1216A, T1216L, T1216P, T1216Q, T1216R, N1217A, N1217E, N1217P, N1217R, N1217S, N1217T, T1218A, T1218C, T1218E, T1218G, T1218Q, T1218S, T1218V, T1218W, S1219I, S1219K, S1219R, S1219V, A1220C, A1220G, A1220P, A1221K, A1221R, A1221V, A1221W, L1222A, L1222E, L1222F, L1222Q, L1222R, L1222V, L1222W, S1224P, S1224W, L1225E, L1225K, L1225V, T1226P, V1227C, V1227D, V1227P, V1227Q, G1229C, G1229E, G1229V, T1230H, T1230K, T1230W, K1231G, V1232E, V1232Q, V1232R, D1234V, S1235D, S1235E, S1235G, S1235P, S1235R, S1235W, S1235Y, V1236A, V1236C, V1236G, V1236P, V1236Q, L1237V, A1238D, A1238E, A1238L, A1238R, A1239R, G1240D, G1240Q, N1243C, I1247R, G1255H, A1259K, V1261R, T1262F, T1262M, T1262R, V1263T, L1264H, L1264R, P1265W, V1273R, I1274F, I1274R, T1275A, T1275W, E1276R, S1277L, S1277T, S1277W, E1278Q, E1278R, D1279I, D1279R, D1279W, H1280C, H1280E, H1280G, V1281I, V1281W, K1284G, T1285A, T1285M, F1286A, F1286E, F1286P, F1286R, T1287C, T1287L, T1287S, T1287W, I1288A, I1288D, I1288G, I1288K, E1293K, E1293L, Q1294E, E1295K, F1296A, E1302G, E1302R, E1302S, R1303S or D1304V.

79. The lactase variant of the preceding embodiment, wherein the variant has a decreased galactose inhibition, preferably measured as a galactose inhibition improvement factor (GI-IF) of at least 1.1, compared to the lactase of SEQ ID NO: 1.

80. The lactase variant of embodiment 1 which comprises one or more of the following substitutions, wherein each position corresponds to a position in SEQ ID NO: 1: V1D, V1F, V1H, V1K, V1L, V1R, E2D, E2G, E2Q, E2V, D3A, D3I, D3N, D3V, A4H, T5A, T5S, R6G, R6H, R6L, R6W, S7L, S7N, S7P, S7T, S9G, S9H, S9W, T10K, T10L, T10S, Q12V, M13C, M13D, M13E, M13H, M13K, M13R, M13W, S14G, S14H, S14T, S14V, S14Y, S15C, S15F, S15I, S15K, S15L, S15P, S15R, S15T, S15V, S15W, T16C, T16I, P17A, P17I, P17S, V19A, V19F, V19I, V19K, V19L, V19N, V19S, V20C, V20F, V20G, V20I, V20L, V20M, V20P, V20Q, V20R, V20T, Y21A, Y21C, Y21D, Y21G, Y21H, Y21M, Y21P, Y21R, Y21T, S22A, S22N, S22R, S22T, S22W, S23A, A24F, A24L, A24R, A24T, A24W, V25D, V25E, V25F, V25G, V25M, V25Q, V25S, V25T, V25W, K28L, K28R, K28S, K28V, K28W, Q29F, Q29G, Q29L, Q29M, Q29R, Q29S, N30M, N30V, N30W, N30Y, R31E, R31M, R31V, T32Q, T32S, S33K, D34F, D34G, D34H, F35A, F35C, F35G, F35V, D36Q, A37N, N38S, W39G, W39S, K40C, K40F, K40G, K40I, K40M, K40P, K40W, F41A, F41C, M42T, L43A, L43G, L43I, L43S, S44M, D45L, D45P, D45V, V47K, V47R, A49C, A49D, A49H, A49T, D51G, D51I, D51K, D51M, A53G, D55C, D55F, D55G, D55H, D55M, D55N, D55P, D55S, D55V, S57A, S57C, S57E, S57G, A58I, A58N, A58Q, A58R, A58T, W59D, W59I, W59K, W59L, W59N, W59P, W59V, Q60E, Q60F, Q60G, Q60L, Q60M, Q60R, Q60S, Q60V, Q60Y, Q61P, V62G, V62N, V62T, V62W, D63G, D63L, D63S, L64E, L64G, H66R, H66T, I70H, I70K, I70P, 170R, T71H, T71L, T71P, T71R, T71S, K73A, K73D, K73G, K73Q, K73V, Y74G, Y74K, S75G, S75L, S75Q, S75R, S75V, Q76G, Q76I, Q76P, S77C, S77D, S77E, S77G, S77H, S77I, S77K, S77L, S77M, S77R, S77T, S77V, S77W, N78C, N78F, N78S, E79H, E79S, E79T, A83E, A83T, L85A, L85C, L85D, L85F, L85N, L85S, L85V, L85W, P86E, P86G, P86Q, P86R, P86V, P86W, P86Y, G87D, G87N, G88A, G88F, G88M, T89C, T89L, T89M, T89N, T89W, T89Y, G90A, G90D, G90L, G90S, G90T, W91E, W91L, W91P, W91Q, W91Y, Y92F, Y92H, Y92I, Y92M, Y92N, Y92S, Y92T, Y92V, Y92W, R93A, R93D, R93F, R93H, R93I, R93L, R93N, R93T, R93V, R93Y, K94A, K94C, K94G, K94R, K94S, K94T, K94V, S95A, S95C, S95D, S95E, S95G, S95I, S95L, S95Q, S95R, F96A, F96C, F96I, F96K, F96L, F96M, F96P, F96V, F96W, T97F, T97S, T97V, I98C, I98H, I98S, I98W, R100T, D101A, D101P, D101V, L102A, L102G, L102M, L102P, L102S, G104C, K105D, K105Q, K105R, K105W, K105Y, R106K, R106V, R106W, I107A, I107F, I107S, A108E, A108V, I109M, I109T, N110A, N110F, N110S, N110T, N110V, N110W, F111A, F111C, F111L, F111Q, F111V, D112A, V114F, V114R, Y115E, M116A, M116C, M116D, M116W, N117K, A118P, T119G, V120A, V120K, W121C, W121D, W121R, W121T, W121V, G124M, V125D, K126E, K126V, T129V, H130A, H130C, H130S, H130T, P131K, Y132E, F137C, F137D, F137L, F137P, S138A, S138D, S138H, S138M, D140V, L141G, T142S, G148K, G149I, G149M, G149Y, E150A, E150C, E150G, E150L, E150N, E150R, N151L, V154E, V154I, V154K, V154L, V154M, V157A, V157G, V157P, V157Q, V157S, E158H, E158K, E158Q, E158V, N159D, N159H, N159T, R160G, L161E, L161K, L161M, L161S, L161W, P162F, P162G, P162N, P162T, P162W, S164A, R165H, W166S, Y167A, Y167C, G169A, G169S, S170Q, G171C, G171F, I172G, I172K, I172P, I172Q, Y173A, Y173H, Y173M, Y173P, Y173S, R174E, R174K, D175Y, V176E, V176K, V176T, T177C, T177E, T177K, L178I, L178Q, L178W, T179A, T179C, T179D, T179H, T179I, T179K, T179L, T179N, T179P, T179S, V180A, V180C, V180D, V180E, V180G, V180M, T181A, T181F, G183W, V184F, V184H, V184P, V184Q, V184R, V184S, V184W, H185G, H185L, H185R, V186A, V186E, V186G, V186N, G187A, G187D, G187H, N188R, N188S, N188V, N188W, N189A, G190F, G190V, V191T, V191Y, I193N, I193Q, I193T, I193V, K194I, T195M, T195W, P196A, P196I, P196M, P196S, P196W, S197C, S197E, S197L, L198I, A199E, A199P, A199T, Q201E, Q201K, N202A, N202M, N202Q, N202S, N202W, G203Q, G204R, N205W, V206C, T207L, K213A, A215D, N216D, D217F, D217G, D217L, D217M, T218D, T218G, T218H, K219A, A220G, A221C, A221D, A222R, N223S, I224G, T225L, L226Q, Q228N, Q228R, F231L, P232T, G234V, G235K, K236M, K236P, T237D, D238K, A240Q, G242K, G242L, G242M, G242P, T243I, A247K, S248F, I251F, I251W, I251Y, A252H, A252P, A252W, A252Y, A257D, A257G, A257N, A257V, T260V, S261A, S261H, S261Y, T262E, T262F, T262P, I263A, T264L, A266D, S267V, P268F, P268R, L270N, W271T, S272T, S272W, I273W, K274Q, N275M, N277F, T280D, V281Q, R282W, T283M, V285H, L286W, V291H, V291L, V291P, V291S, L292A, L292D, L292E, L292Q, L292S, L292V, D293C, D293I, D293W, T294C, T294M, T294Q, T294S, Y295F, Y295G, Y295L, Y295S, D296F, D296H, D296K, T297I, T297S, E298I, E298L, E298M, E298R, E298T, Y299F, Y299S, F301C, R302I, R302K, R302M, R302T, W303A, W303C, W303D, W303F, W303T, T304D, T304E, T304I, T304K, T304P, T304S, T304W, G305E, G305L, G305M, G305N, G305P, G305T, D307A, D307E, D307F, D307S, D307V, D307W, A308G, A308I, T309C, T309D, T309E, T309I, T309K, T309V, S310A, S310C, S310G, S310H, S310I, S310L, S310M, S310N, S310R, S310V, G311F, G311L, F312H, F312L, F312V, L314A, L314V, L314Y, N315S, G316N, G316R, G316W, G316Y, E317V, E317W, E317Y, K318C, K318E, K318F, K318H, K318M, K318N, K318P, K318Q, K320E, K320I, K320V, K320W, L321H, L321M, L321Y, K322C, K322I, V324E, V324G, V324T, S325G, M326T, M326V, H328C, H328D, H328F, H328G, H3281, H328L, H328M, H328R, H328T, G331V, A335C, A335G, A335L, A337D, A337E, A337G, A337L, N338P, N338R, R340A, R340L, A341M, I342A, I342G, I342P, E343A, E343N, E343R, E343T, E343Y, R344G, R344Y, Q345F, Q345N, Q345S, V346A, V346I, V346S, E347A, E347D, E347I, I348A, I348D, I348G, I348M, I348Q, I348R, L349A, L349E, L349S, L349V, Q350G, Q350N, Q350W, K351R, K351W, M352L, M352T, G353K, V354E, V354G, V354S, V354W, N355H, I357T, T359E, T359L, T360V, N362S, N362T, P363A, P363D, P363I, P363L, P363M, P363S, P363V, A364C, A364E, A364F, A364G, A364I, A364M, A364P, A364V, A364W, A365C, A365I, A365P, A365V, A365W, K366D, K366I, K366L, K366M, K366P, K366S, K366V, A367I, A367N, A367Q, L368A, L368Q, L368S, L368V, L368Y, I369A, I369D, I369E, I369G, I369K, I369M, I369V, I369W, D370L, D370Q, D370R, D370T, V371F, V371G, V371I, V371L, V371Q, C372P, N373G, N373L, N373R, E374L, K375D, K375I, K375N, K375Q, K375S, G376A, G376S, V377A, V377M, V377T, L378I, L378P, L378Y, V379A, V379C, V379M, V379N, V380P, V380S, E381A, E381C, E381G, E381Q, E381T, V383A, M386G, M386N, M386S, M386V, W387H, N388E, N388L, R389A, R389C, R389E, R389K, R389M, R389Q, R389S, R389T, R389V, S390C, S390D, S390H, S390P, S390T, G393E, G393R, G393V, T395A, T395C, T395F, E396K, E396L, E396V, E396W, Y398M, G399S, K400A, K400C, K400M, K400N, K400T, K400V, W401F, W401K, F402W, F402Y, G403A, G403D, G403H, G403K, G403Q, G403T, G403V, G403Y, Q404F, Q404L, Q404M, Q404P, Q404S, Q404V, A405C, A405E, A405K, A405R, A405V, I406C, I406D, I406N, A407C, A407G, A407Q, N410R, N410Y, A411E, A411R, A411S, V412S, G414M, G414N, G414T, G415A, G415Q, D416M, K417F, K417R, A422P, K423L, K423R, L426C, T427D, T427F, T427Q, T427S, T427W, T429D, T429P, I430L, I430M, N431L, N431M, R432F, R432G, R432Q, R432Y, D433C, D433G, D433I, D433P, D433Q, D433W, R434N, R434T, R434V, N435E, N435F, N435H, N435L, N435V, A436C, A436G, A436L, A436M, P437K, P437L, P437W, S438G, V439C, V439K, I440S, I440T, I440V, M441R, M441T, W442E, W442G, S443C, S443G, S443M, S443Q, L444E, L444F, L444V, N446D, M448C, M448E, M448L, M449D, M449E, M449F, M449T, M449V, M449W, M449Y, E450F, E450S, E450T, E450V, E450W, G451C, G451F, G451L, G451P, G451V, G451W, I452K, I452L, I452Q, I452S, I452V, S453C, S453G, S453H, S453L, S453M, S453N, S453P, S453Q, S453R, S453V, G454L, G454W, S455E, S455M, S455P, S455R, S455V, V456D, V456E, V456F, V456K, S457H, S457Q, S457T, S457V, G458A, G458P, G458S, F459C, F459R, P460M, P460Y, A461G, A461M, A461S, T462E, T462L, A464W, K465C, K465G, K465L, K465Q, K465V, K465Y, L466G, L466P, V467T, A468D, W469L, W469R, K471F, K471Q, K471Y, A472G, A472Y, A473M, A473P, D474C, D474E, D474M, D474W, S475E, S475T, T476L, T476S, R477A, R477G, R477L, P478A, P478D, P478V, M479G, T480Q, K485E, K485R, K487A, K487C, K487F, K487S, K487W, A488C, A488L, A488V, S493E, S493G, S493H, S493L, S493Q, N494A, N494I, N494M, N494R, N494V, T495K, G497D, N499T, G505D, G505H, G505L, G505R, G505S, G505V, V506C, V506E, V506R, V507F, V507P, V507R, G508C, G508E, T509A, T509D, T509E, T509I, T509K, T509M, T509Q, T509S, T509V, T509Y, N510A, N510F, N510I, Y511A, S512C, S512E, S512F, S512I, S512M, S512Q, S512T, S512V, S512Y, D513G, D513P, D513R, A515E, A515F, A515G, A515S, N516C, N516E, N516G, N516I, N516M, N516Q, N516S, N516T, N516V, Y517G, Y517I, D518Y, K519C, K519E, K519F, K519G, K519I, K519L, K519M, K519N, K519Q, K519S, K519T, I520A, I520C, I520G, I520H, I520M, I520N, I520Q, I520S, I520T, I520W, I520Y, R521A, R521C, R521K, R521Q, R521V, T523A, T523M, H524R, H524V, P525R, S526G, W527A, W527E, W527G, W527H, W527N, W527R, W527S, A528E, A528G, A528L, I529G, A535E, A535G, A535I, A537M, A537P, A537S, I538H, I538M, S540E, N545G, T547K, G549D, G549P, G549W, G550Q, G550R, S553P, S553R, D555E, D555P, K556C, K556R, L558E, L558H, L558P, T559G, T559Q, T559V, T559Y, S560P, S560V, S564A, S564C, S564F, S564W, A565C, A565D, A565E, A565F, A565G, A565I, A565L, A565M, A565R, A565S, A565T, A565V, A565W, G567N, G567Q, G567V, A570G, A570L, A572W, S574V, A575D, Y577R, D578T, V580D, Q581T, Q581Y, R582A, R582G, R582I, R582L, D583V, D583W, F584I, F584W, A586D, T588P, Y589I, Y589Q, W591F, T592L, G593C, F594C, L597D, L597T, G598N, P600A, P600E, P600G, P600S, N604E, N604S, G605R, G607Q, G607S, S608E, S608I, S608N, S608Q, S608T, G609A, G609S, A610T, G612V, S613A, W614A, P615V, N619I, N619L, N619S, Y621W, V625A, V625E, V625F, V625M, V625Y, T627K, T627Q, A628C, A628D, A628N, G629T, F630C, F630Y, P631D, P631Y, K632C, K632D, K632G, D633G, T634F, T634S, T634V, Y636K, F637C, F637G, F637I, F637S, F637T, F637V, Q639R, N643R, D644Y, D645S, D645V, V646C, V646L, V646N, V646R, V646S, H647G, H647V, T648C, L649V, H650F, I651T, I651V, L652C, L652D, L652V, L652W, P653Q, A654D, A654R, W655F, W655R, N656K, E657K, E657R, E657V, A661E, A661G, A661H, A661K, A661L, A661M, A661Q, A661W, K662H, K662S, K662V, K662W, K662Y, N667L, N667R, P669A, P669E, P669L, P669R, P669T, P669W, V672L, Y673S, T674D, T674M, T674Q, D675A, D675V, D675Y, A676L, A676P, A677T, V679Q, V679S, K680I, K680N, Y682V, F683W, T684R, K686E, G687P, S688K, S688L, T689G, T689W, E690D, K691E, K691H, K691P, K691R, R692G, R692H, R692I, R692L, R692P, R692S, R692T, R692V, R692W, L693M, L693P, I694L, I694W, G695C, G695K, G695L, G695R, G695W, E696R, K697A, K697G, K697R, K697V, K697W, S698D, S698E, S698M, S698Q, S698R, T700A, T700D, T700Y, K701A, K701D, K701G, K701H, K701L, K701M, K701P, K701S, T703K, A705D, A705K, A705P, A705R, A705W, A706C, A706T, A706W, Y708F, Y708L, Y710E, Y710T, Y710V, Q711A, Q711D, Q711T, V712M, V712P, V712Q, Y713E, Y713G, E714H, E714I, E714K, E714N, G715K, A716M, A716P, A716R, D717G, D717L, D717S, K718A, K718Q, K718T, K718W, K718Y, D719E, D719S, D719T, D719V, S720A, S720M, S720R, S720Y, T721D, T721H, T721K, T721L, T721N, T721P, T721Q, T721S, T721V, T721W, T721Y, A722Q, A722T, H723G, H723P, K724A, K724G, N725A, N725I, M726D, M726Q, Y727L, L728C, L728K, L728S, L728W, T729A, T729E, T729M, N731E, N731Q, V732R, P733W, W734G, W734R, A735Q, A735R, A735S, A735T, G737F, G737K, G737N, G737Y, T738L, T738S, I739E, I739K, S740D, S740F, A741I, A741P, A741S, A741V, E742V, A743E, A743I, Y744L, Y744R, D745C, D745N, D745R, E746C, E746K, E746T, N747E, N747F, N747P, R749M, L750M, L750P, L750Q, L750S, I751C, I751H, P752C, P752Y, G754H, G754I, S755T, T756N, T756Q, E757A, G758V, N759S, N759V, A760G, S761A, V762W, T765P, G766M, A769F, A769G, A769I, A769N, A769V, A769Y, K770H, L771A, K772A, K772C, K772P, K772W, A773C, A773M, A773R, D774A, A775V, D776R, D776V, R777H, R777S, R777T, K778F, K778G, K778N, K778R, T779I, T781A, T781E, T781F, T781G, T781P, T781Y, A782C, A782N, A782Q, A782Y, D783A, K785C, K785I, K785S, K785V, K785W, K785Y, D786V, L787D, L787K, Y789V, I790R, I790V, E791D, E791F, E791S, E791V, E791W, V792S, V792Y, D793C, D793Y, V794D, D796S, D796T, A797H, N798I, N798Q, G799D, G799K, G799L, G799Y, H800F, H800G, H800V, I801W, V802E, V802I, V802S, V802Y, P803G, P803S, P803Y, D804E, A805C, A805F, A805I, A805N, A805P, A806F, A806I, A806Q, N807F, R808F, R808G, R808I, R808P, V809L, V809M, V809P, T810L, T810P, T810Q, T810R, T810Y, F811L, V813H, V813W, V813Y, K814G, K814P, G815F, G815M, G815P, A816D, A816N, A816W, G817H, K818D, K818F, K818L, K818Q, K818R, K818S, K818W, K818Y, L819F, V820C, V820R, V820W, G821I, V822A, V822D, V822E, V822T, D823E, N824A, G825A, S826A, S826G, S826I, S826W, P828G, D829C, D829I, D829S, H830Q, D831A, D831M, D831P, S832P, S832R, S832W, Y833C, Y833D, Y833E, Y833N, Q834M, A835D, A835H, A835K, D836C, D836E, D836Q, D836R, D836S, D836T, D836V, D836Y, R838N, R838S, K839A, K839D, K839L, K839N, A840G, A840I, A840P, A840V, F841I, F841K, S842A, K844A, K844Y, A847T, I848M, Q850L, S851C, S851D, S851E, S851T, T852L, K853P, E854C, E854I, A855K, A855V, A855Y, I858D, I858G, I858K, I858P, V860Y, T861F, T861I, T861Q, T861V, T861W, A862C, A864K, D865A, D865G, G866H, G866R, L867W, S870E, S870M, S870T, V872C, V872G, K873G, K873Y, I874G, A875R, V879S, T882A, T882M, T882R, S883L, E885V, K886L, K886V, K886W, T887A, T887D, T887G, T887N, V888A, R889G, Y892P, Y893E, Y893G, S894D, S894G, Y897V, Y898T, K900E, K900G, T901Q, T901R, T901Y, G902A, G902D, G902F, G902P, G902Q, G902R, G902S, G902W, N903D, N903I, K904D, K904E, K904M, K904N, K904R, K904S, K904V, K904W, P905A, P905C, P905R, P905V, P905W, P905Y, I906A, I906T, I906W, L907F, L907S, P908C, P908D, P908G, P908I, P908L, P908T, S909E, S909F, S909G, S909W, S909Y, D910I, D910W, V911A, E912A, E912K, E912L, E912T, E912V, V913G, V913R, R914A, R914E, R914F, R914I, R914K, R914V, R914W, R914Y, Y915A, Y915C, Y915G, Y915I, Y915M, Y915Q, Y915V, S916G, D917C, D917E, D917F, D917L, D917M, D917Q, D917S, G918E, G918H, G918T, G918V, T919D, T919K, T919Q, T919W, S920P, S920V, D921C, D921V, R922A, R922M, R922V, R922W, Q923A, Q923E, Q923L, Q923M, Q923V, N924Q, N924S, V925C, V925E, V925G, V925K, V925N, V925W, T926P, T926R, W927P, D928A, D928H, D928Q, A929P, V930E, V930I, V930T, S931P, S931R, D933R, Q934S, Q934V, I935A, I935C, I935D, I935E, I935L, I935P, I935V, I935W, A936I, A936L, A936Q, A936R, A936Y, K937G, K937I, K937M, K937P, K937Q, A938C, A938H, A938N, A938T, A938V, A938W, G939D, G939K, S940C, S940E, S940R, S940V, F941C, F941M, S942A, S942E, S942K, S942P, S942V, V943A, V943G, V943Q, T946A, T946L, T946P, T946W, V947G, V947L, V947P, V947R, V947T, A948I, A948W, G949A, G949V, Q950K, Q950W, K951D, K951G, K951P, K951S, K951W, K951Y, I952Q, S953F, S953M, S953N, S953R, V954D, V954Q, R955A, R955C, R955K, R955Q, R955W, V956A, V956D, V956G, V956H, V956I, V956M, V956Q, V956W, T957D, T957S, T957W, I959A, I959L, I959S, I959V, I959Y, D960P, D960S, E961D, E961F, E961K, E961S, E961T, I962A, I962C, I962D, I962G, I962K, I962N, G963C, G963E, G963P, L965C, L965E, L965G, L965K, L965M, L965P, L965Q, L965S, L965V, L965Y, L966A, L966K, L966N, L966Q, L966S, L966V, N967D, N967I, N967L, N967M, N967P, N967T, N967V, Y968G, Y968L, Y968Q, Y968V, S969C, S969D, S969G, S969H, S969I, S969L, S969M, S969P, S969Q, S969Y, A970I, A970L, S971G, S971V, S971W, P973C, P973D, P973K, P973N, P973Q, P973R, P973V, P973W, P973Y, V974C, V974E, V974G, V974N, V974T, V974Y, G975F, G975K, T976D, T976F, T976G, T976K, T976L, T976P, T976S, P977C, P977K, P977R, P977T, P977Y, A978F, A978G, A978M, A978N, A978R, A978S, A978Y, V979G, V979N, V979Y, L980A, L980F, L980H, L980I, L980K, L980N, L980Q, L980T, L980Y, P981L, P981M, R984P, P985F, P985H, P985K, P985L, P985W, A986C, A986I, A986L, A986M, A986W, V987A, V987C, V987F, V987K, L988V, P989M, P989W, D990F, D990P, D990W, G991C, G991F, G991H, G991K, G991P, G991Y, T992M, T992N, V993G, S995E, S995L, S995R, A996R, N997K, N997V, F998M, F998W, V1000W, D1001L, D1001S, W1002A, W1002D, W1002E, W1002H, W1002N, W1002P, W1002Q, W1002S, T1003F, T1003G, T1003N, T1003R, T1003S, T1003W, T1003Y, K1004D, K1004E, K1004S, P1005I, P1005N, P1005V, P1005Y, A1006C, A1006N, A1006P, A1006S, A1006V, A1006W, A1006Y, D1007C, D1007L, D1007P, D1007V, T1008G, Y1010A, Y1010P, Y1010R, Y1010T, N1011A, N1011S, N1011T, N1011W, T1012H, T1012I, T1012Q, A1013D, A1013Q, A1013V, G1014I, G1014M, G1014V, G1014W, T1015A, V1016D, V1016P, K1017E, K1017G, V1018K, V1018L, V1018R, V1018S, T1021C, T1021E, T1021G, A1022H, V1024G, V1024H, V1024K, V1024R, V1024S, V1024W, G1026E, G1026S, G1026V, G1026Y, K1027Q, K1027R, E1028G, E1028T, F1029I, F1029K, F1029P, F1029V, K1030D, K1030H, K1030M, K1030W, V1031H, V1031K, A1033V, T1034G, T1034H, T1034N, I1035D, I1035G, I1035Q, R1036G, R1036L, R1036T, R1036Y, V1037C, Q1038D, R1039V, S1040M, Q1041P, V1042N, T1043F, T1043G, T1043R, I1044A, I1044L, S1046M, V1048C, V1048F, V1048G, V1048I, V1048M, V1048Q, N1051A, N1051E, N1051K, A1052C, A1052K, A1052R, L1053A, L1053W, R1054C, R1054L, L1055R, L1055T, Q1057A, Q1057E, Q1057P, N1058R, N1058S, P1060G, P1060N, P1060Q, A1061G, A1061K, A1061W, D1062A, D1062G, D1062I, D1062M, K1063M, Q1064M, Q1064R, Q1064T, Q1064V, S1065A, S1065C, D1066A, D1066G, D1066V, T1067G, T1067M, L1068P, L1068Q, L1068Y, D1069R, A1070T, I1071M, K1072P, K1072S, D1073F, D1073W, G1074L, G1074R, S1075G, S1075L, T1076E, T1076S, T1077R, V1078D, V1078W, D1079G, D1079L, N1081E, T1082E, T1082F, T1082G, T1082K, G1083E, G1083F, G1083L, G1084M, G1084V, G1084W, G1084Y, G1085P, G1085R, G1085S, A1086H, A1086K, A1086R, N1087A, N1087E, N1087I, N1087R, P1088D, P1088E, P1088G, P1088R, P1088W, S1089G, S1089K, S1089Q, S1089V, A1090F, W1091Y, N1093A, N1093G, N1093L, N1093P, N1093Q, W1094D, W1094E, W1094P, W1094R, W1094T, A1095P, A1095T, A1095W, Y1096D, Y1096L, S1097E, S1097K, S1097T, K1098D, K1098F, K1098G, K1098Q, K1098S, A1099D, A1099S, A1099V, A1099W, G1100E, G1100H, G1100N, H1101K, H1101Q, H1101R, H1101V, N1102H, N1102Q, N1102T, T1103A, T1103E, T1103S, T1103W, E1105L, E1105S, E1109A, E1109D, E1109L, E1109W, A1111G, A1111S, E1113G, E1113P, E1113V, Q1114E, Q1114L, Q1114R, Q1114S, Q1114T, Q1114V, Q1115A, Q1115K, Q1115L, Q1115P, L1116D, L1116G, L1116H, L1116K, L1116W, G1117E, G1117I, G1117M, G1117R, G1117S, G1117T, G1117V, G1117W, Q1118M, Q1118W, I1119D, I1119E, I1119N, I1119S, V1120N, V1120S, M1121G, M1121K, M1121N, M1121P, M1121S, F1123E, F1123R, F1124V, F1124W, R1125D, R1125F, R1125K, R1125V, R1125W, S1127I, S1127Q, N1128A, A1129E, A1129R, V1130A, V1130R, R1131W, F1132M, F1132P, F1132Q, P1133D, P1133G, P1133L, P1133R, A1135E, A1135K, A1135L, A1135W, G1136P, K1137G, K1137P, K1137R, K1137S, K1137T, K1137V, T1138R, T1138Y, I1140C, I1140P, I1140R, Q1141A, Q1141G, Q1141K, Q1141N, Q1141T, I1142E, I1142R, I1142Y, S1143G, A1144D, A1144N, A1144P, A1144S, A1144T, A1144V, A1144W, D1145E, D1145R, D1145T, G1146A, G1146K, G1146R, G1146V, N1148H, N1148I, N1148P, N1148Q, N1148R, N1148S, N1148T, W1149C, W1149G, W1149K, W1149S, W1149V, T1150K, T1150P, D1151C, D1151W, L1152A, L1152C, L1152E, L1152Q, L1152W, A1153E, A1153G, A1153L, A1154D, A1154E, A1154R, I1158S, I1158W, A1159E, A1159I, Q1161P, Q1161S, E1162A, E1162C, E1162Q, E1162W, E1165M, E1165S, R1166Q, V1167A, V1167R, K1168L, K1168R, P1169M, P1169S, Y1170K, Y1170M, Y1170V, Y1172H, Y1172S, D1173G, D1173T, F1174D, A1175S, G1178M, G1178Q, G1178S, G1178T, A1179L, A1179Q, A1179W, T1180A, T1180G, T1180I, T1180L, T1180M, T1180Q, T1180S, T1180Y, F1181E, F1181V, V1182M, K1183A, K1183E, K1183T, K1183V, V1184C, V1184E, V1184K, V1184L, V1184P, V1184Q, V1184Y, T1185Q, T1185V, V1186S, N1188V, N1188W, A1189C, A1189K, A1189T, A1189V, D1190R, D1190T, D1190Y, T1191L, T1193G, P1194E, P1194W, V1197A, C1199D, A1200G, T1203K, E1204G, E1204S, I1205V, K1208R, K1208V, K1208W, T1209A, T1209C, T1209E, T1209K, T1209N, T1209Q, T1209R, T1209W, A1210D, A1210G, A1210K, A1210L, A1210Q, A1210R, A1210T, A1210W, T1211C, T1211D, T1211E, T1211G, T1211H, T1211K, T1211P, T1211Q, T1211R, T1211S, T1211V, K1213A, K1213D, K1213S, K1213T, F1214A, F1214E, F1214K, F1214L, F1214P, F1214S, F1214V, V1215D, V1215E, V1215L, V1215Q, V1215W, T1216L, T1216P, T1216Q, T1216R, N1217A, N1217E, N1217P, N1217R, N1217S, N1217T, T1218A, T1218C, T1218E, T1218G, T1218Q, T1218S, T1218V, T1218W, S1219I, S1219K, S1219R, S1219V, A1220C, A1220G, A1220P, A1221K, A1221R, A1221V, A1221W, L1222A, L1222E, L1222F, L1222Q, L1222R, L1222V, L1222W, S1224P, S1224W, L1225E, L1225K, L1225V, T1226P, V1227C, V1227D, V1227P, V1227Q, G1229C, G1229E, G1229V, T1230H, T1230K, T1230W, K1231G, V1232E, V1232Q, V1232R, D1234V, S1235D, S1235E, S1235G, S1235P, S1235R, S1235W, S1235Y, V1236A, V1236C, V1236G, V1236P, V1236Q, L1237V, A1238D, A1238E, A1238L, A1238R, A1239R, G1240D, G1240Q, N1243C, I1247R, G1255H, A1259K, V1261R, T1262F, T1262M, T1262R, V1263T, L1264H, L1264R, P1265W, V1273R, I1274F, I1274R, T1275A, T1275W, E1276R, S1277L, S1277T, S1277W, E1278Q, E1278R, D1279I, D1279R, D1279W, H1280C, H1280E, H1280G, V1281I, V1281W, K1284G, T1285A, T1285M, F1286A, F1286E, F1286P, F1286R, T1287C, T1287L, T1287S, T1287W, I1288A, I1288D, I1288G, I1288K, E1293K, E1293L, Q1294E, E1295K, F1296A, E1302G, E1302R, E1302S, R1303S or D1304V.

81. The lactase variant of the preceding embodiment, wherein the variant has a decreased galactose inhibition, preferably measured as a galactose inhibition improvement factor (GI-IF) of at least 1.1, compared to the lactase of SEQ ID NO: 1.

82. The lactase variant of embodiment 1 which comprises one or more of the following substitutions, wherein each position corresponds to a position in SEQ ID NO: 1: V1D, V1F, V1H, V1L, E2D, E2Q, E2V, D3I, D3S, D3V, D3W, T5D, T5S, R6A, R6F, R6M, R6P, S7D, S7I, S7N, S7P, S9H, S9P, T10L, T10P, T10S, T11L, T11P, Q12R, M13C, M13D, M13F, M13K, M13R, S14V, S15C, S15I, S15K, S15P, S15R, S15T, S15V, S15Y, T16C, P17D, P17I, P17L, P17T, V19G, V19K, V20C, V20G, V20K, V20L, V20M, V20P, V20Q, V20R, V20W, Y21C, Y21P, Y21R, Y21T, S22E, S22G, S22N, S22T, S22W, S23C, S23L, S23M, S23R, A24F, A24L, A24R, A24T, A24W, V25D, V25K, V25M, V25Q, V25S, D26C, D26I, D26L, S27A, S27C, S27F, S27G, S27H, S27Y, K28C, K28L, K28V, Q29G, Q29L, Q29M, Q29R, Q29V, Q29W, N30A, N30M, N30V, R31E, R31G, R31I, R31M, R31V, T32S, S33C, S33N, S33Q, S33R, D34C, D34E, D34G, D34H, D34L, D34S, D34W, D34Y, F35C, F35E, F35K, F35N, D36Q, N38S, W39G, W39S, K40C, K40D, K40F, K40G, K40I, K40N, K40W, F41A, F41C, F41G, F41I, F41Q, M42E, M42N, M42T, L43A, L43C, L43I, L43S, L43T, L43V, S44C, D45A, D45P, V47K, V47R, A49D, A49H, A49R, A49S, A49T, A49V, D51G, D51I, D51K, D51P, D51V, A53C, A53R, A53S, A53V, A53W, D55C, D55F, D55G, D55H, D55M, D55P, D55S, S57A, S57C, S57E, S57G, A58D, A58G, A58Q, A58T, W59I, W59K, W59P, W59V, Q60K, Q60M, Q60R, Q60S, Q60Y, Q61P, V62N, V62S, V62T, V62W, D63G, L64E, L64G, H66L, H66R, H66Y, Y68P, I70A, I70K, T71C, T71K, T71Q, K73D, Y74K, S75G, S75L, S75Q, S75R, S75V, Q76G, Q76I, Q76K, Q76V, S77C, S77D, S77E, S77F, S77G, S77H, S77K, S77L, S77M, S77R, S77T, S77V, N78C, N78E, N78K, N78Q, N78R, N78S, E79H, E79Q, E79S, E79T, A80K, E81A, E81Q, A83T, L85A, L85F, L85N, L85S, L85V, L85W, P86G, P86N, P86Q, P86R, P86V, P86W, P86Y, G87D, G87N, G87Q, G88F, G88Q, G88S, T89H, T89K, T89M, T89Y, G90A, G90L, G90S, G90T, G90V, W91E, W91L, W91P, W91Q, W91R, W91Y, Y92F, Y92H, Y92I, Y92M, Y92N, Y92S, Y92T, Y92V, R93A, R93D, R93F, R93H, R93I, R93L, R93N, R93T, R93V, R93Y, K94A, K94C, K94G, K94S, K94T, K94V, S95A, S95C, S95D, S95G, S95I, S95Q, S95R, F96A, F96C, F96I, F96K, F96M, F96P, F96S, F96V, F96W, T97F, T97V, I98C, I98H, I98W, R100T, D101A, D101P, D101V, L102P, R106V, R106W, I107F, I107G, I107S, A108E, A108S, I109M, N110A, N110S, N110T, N110V, N110W, F111A, F111L, F111Q, D112F, D112T, G113S, V114F, V114R, Y115E, M116C, N117W, A118K, A118P, A118Y, T119A, V120A, V120K, W121D, W121R, W121V, W121Y, F122A, F122S, F122Y, N123P, G124E, G124M, G124Q, G124R, V125E, V125I, G128D, T129E, H130A, H130C, H130S, H130T, Y132C, Y132E, P136R, P136Y, F137C, F137L, S138A, S138L, S138R, F139Q, D140V, L141G, T142S, T142V, K146A, G148T, G149E, G149M, G149Q, G149Y, E150C, E150G, E150L, E150R, I153A, V154I, V154K, V154L, V155F, V157L, V157P, V157S, E158G, E158H, E158V, N159H, N159S, N159T, L161K, L161M, L161S, L161W, P162N, P162T, P162W, S164A, R165H, W166S, Y167A, Y167C, G169D, S170L, S170Q, G171C, G171T, I172K, I172P, Y173H, Y173M, Y173W, R174E, R174K, V176E, V176K, V176T, T177K, L178I, L178Q, L178W, T179A, T179C, T179D, T179I, T179K, T179N, T179P, T179S, V180A, V180D, V180G, T181A, T181F, D182F, G183W, V184F, V184P, V184R, V184S, V184W, H185G, H185L, V186E, V186N, G187D, G187H, N188E, N188R, N188V, N189E, G190C, G190F, I193N, I193Q, K194I, T195S, T195W, P196A, P196I, S197C, S197E, S197L, L198R, L198V, A199E, A199K, A199P, Q201E, N202D, N202L, N202Q, N202R, N202S, N202T, N202W, G203C, G203K, G203S, G203V, G203W, G203Y, G204A, G204K, G204Y, N205E, N205G, N205H, N205L, V206I, V206K, V206S, T207A, T207C, T207G, T207I, T207K, T207Q, M208S, N209D, N209K, N209V, L210G, L210I, L210Q, L210V, T211A, T211F, T211Q, T211R, T212E, T212G, T212H, T212L, T212S, K213C, K213D, K213F, K213L, K213T, K213Y, V214A, V214C, V214W, A215D, A215E, A215I, A215K, A215L, A215Q, A215R, A215V, D217F, D217L, D217M, D217T, T218G, K219F, K219H, K219M, A220C, A220G, A220L, A220V, A221E, A221L, A221R, A222D, A222I, A222L, A222P, A222R, A222W, N223E, N223F, N223K, N223L, N223S, N223T, L226M, L226Q, Q228N, Q228R, T229A, T229C, T229D, T229G, T229M, T229N, T229Q, T229R, T229V, V230L, V230M, V230R, V230S, F231E, F231G, F231I, F231L, F231V, F231W, F231Y, P232G, P232M, P232S, K233C, K233E, K233F, K233L, K233P, K233R, G234A, G234C, G234D, G234E, G234L, G234Q, G234R, G234V, G234Y, G235H, G235I, G235K, G235M, G235Q, G235T, G235W, G235Y, K236A, K236D, K236L, K236M, K236P, K236R, K236S, K236W, K236Y, T237D, T237K, T237R, D238A, D238E, D238F, D238G, D238M, D238P, D238R, A239E, A239G, A239I, A239T, A240C, A240L, A240P, A240T, A240Y, I241T, G242K, G242L, G242M, G242P, G242Y, T243I, T243M, T243R, V244E, V244R, T245E, T245G, T245Q, T245R, T246G, T246K, A247E, A247K, A247P, A247R, A247S, A247V, A247W, S248A, S248E, S248L, S248Q, S248T, K249A, K249D, K249G, K249H, K249L, K249N, K249P, K249Q, K249V, K249Y, S250H, I251L, I251W, I251Y, A252F, A252W, A252Y, G254D, G254F, G254I, G254L, G254M, G254Q, G254R, G254W, A255K, A255S, A255Y, S256A, S256C, S256K, S256M, S256N, S256R, S256W, S256Y, A257D, A257I, A257N, A257V, D258A, D258L, D258W, V259E, V259T, T260D, T260G, S261D, S261H, S261R, S261W, T262D, T262W, T264F, T264M, T264Q, T264Y, A265I, A265S, A266D, A266G, A266K, A266Q, A266T, S267A, S267M, S267N, S267P, S267R, S267V, P268F, P268G, P268R, P268W, P268Y, K269V, K269Y, L270D, L270N, S272G, S272T, S272W, I273L, I273R, N275W, N277F, N277R, L278H, L278I, L278K, L278M, L278Q, L278R, L278V, Y279W, T280A, T280E, T280F, T280Q, V281I, V281Q, R282E, R282F, R282H, R282I, R282K, R282N, R282S, R282V, R282W, T283R, T283V, E284A, E284D, E284H, E284L, E284R, E284Y, V285T, L286F, L286N, L286T, L286W, G288S, V291D, V291F, V291P, V291T, V291Y, L292D, L292H, L292V, D293C, D293I, D293S, D293W, T294Q, T294S, Y295F, Y295W, D296F, D296H, D296K, D296R, D296V, T297I, E298I, E298L, E298R, F301C, R302I, R302N, W303A, W303C, W303D, W303F, W303S, T304D, T304E, T304I, T304K, T304P, G305L, G305N, G305P, G305T, G305W, F306K, F306L, D307A, D307E, D307G, D307S, D307W, A308E, A308M, A308Y, T309C, T309D, T309E, T309I, T309S, T309V, S310A, S310H, S310L, S310M, S310N, S310V, G311V, F312H, F312R, L314A, L314C, L314T, L314V, N315S, G316R, G316S, E317K, E317W, K318C, K318N, K320C, K320E, K320F, K320I, K320L, K320R, K320T, K320V, K320W, L321M, K322F, K322I, K322P, K322S, V324T, S325D, S325G, S325T, M326E, M326G, M326T, M326Y, H328C, H328F, H328G, H328L, H328M, H328R, H328T, D329S, G331V, A335C, A335G, A335L, A337E, A337G, A337L, N338P, R340A, R340C, R340L, A341M, A341S, I342G, I342K, I342P, E343N, E343T, E343Y, R344G, Q345F, Q345G, Q345K, Q345N, Q345S, V346A, V346C, V346I, V346S, E347A, E347C, E347F, E347I, E347R, I348D, I348M, I348Q, I348R, L349A, L349Q, L349S, Q350G, Q350N, K351V, M352G, M352T, M352W, G353K, V354E, V354S, V354W, N355M, N355W, I357T, T359E, T359L, T360V, N362S, N362T, P363A, P363D, P363I, P363L, P363M, P363S, P363V, A364C, A364E, A364F, A364G, A364I, A364M, A364P, A364V, A365C, A365E, A365I, A365P, A365V, A365W, K366A, K366D, K366L, K366M, K366P, K366S, K366V, A367Q, L368A, L368E, L368Q, L368S, L368Y, I369A, I369D, I369E, I369G, I369K, I369M, I369R, I369V, I369W, D370L, D370Q, D370S, V371D, V371F, V371L, V371Q, V371S, C372P, N373G, N373L, E374R, K375D, K375S, G376A, G376S, V377A, V377M, V377T, V379A, V379N, V380P, E381A, E381G, E381Q, E381T, V383A, V383K, V383L, M386G, M386N, M386S, M386V, W387H, W387L, N388A, N388E, N388L, N388R, R389A, R389C, R389E, R389N, R389Q, R389S, R389T, S390C, S390D, S390G, S390P, S390Q, S390T, S390V, K391E, N392D, G393A, G393E, G393N, G393R, G393S, G393V, N394L, T395C, T395F, T395H, T395M, T395N, T395S, T395W, E396K, E396L, E396M, E396V, E396W, Y398M, Y398N, G399S, K400A, K400C, K400N, K400P, K400Q, K400S, K400T, K400V, W401F, W401H, W401K, W401L, F402W, F402Y, G403A, G403D, G403K, G403P, G403Q, G403S, G403V, G403Y, Q404F, Q404H, Q404L, Q404M, Q404P, Q404R, Q404S, Q404V, A405C, A405E, A405H, A405K, A405P, A405R, A405T, I406C, I406D, I406N, A407C, A407G, A407Q, A407T, G408I, G408M, G408N, G408W, D409N, N410R, N410Y, A411E, A411R, A411S, A411V, L413I, L413P, L413T, G414A, G414C, G414M, G414N, G414R, G415Q, G415R, D416I, D416R, D416Y, K417C, K417F, K417G, K417R, K417T, D418P, D418R, E419M, E419R, E419W, T420E, T420F, T420G, T420K, T420R, T420V, W421L, W421Q, A422P, A422T, K423D, F424C, F424L, F424N, L426C, L426M, L426Q, T427D, T427F, T427G, T427K, T427M, T427P, T427Q, T427S, T427W, S428F, S428K, T429D, I430C, I430L, I430M, I430Q, I430S, I430T, N431D, N431E, N431M, N431Y, R432A, R432E, R432N, R432Q, R432Y, D433G, D433I, D433P, D433Q, D433W, R434N, R434S, R434V, N435E, N435F, N435K, N435L, N435R, N435V, N435W, A436G, A436L, P437A, P437Q, P437R, P437V, V439C, V439E, V439G, V439I, V439K, V439T, V439Y, I440F, I440K, I440R, I440W, M441A, M441E, M441Q, W442E, W442G, W442P, W442R, S443C, S443G, S443M, S443Q, S443Y, L444D, L444E, L444F, L444G, L444K, L444Q, L444V, L444W, G445A, G445C, G445V, N446D, N446T, M448A, M448C, M448D, M448I, M448L, M448P, M448Q, M448S, M448V, M449D, M449E, M449T, M449W, M449Y, E450F, E450S, E450T, E450V, E450W, G451C, G451F, G451L, G451P, G451V, G451W, I452G, I452K, I452L, I452M, I452Q, I452S, I452V, S453F, S453H, S453L, S453N, S453Q, G454L, G454W, S455E, S455K, S455M, S455P, S455R, S455W, V456D, V456E, V456F, V456K, V456L, S457H, S457K, S457P, S457Q, S457T, S457V, G458A, G458P, G458S, G458V, G458W, F459C, F459N, F459R, F459S, P460Y, A461D, A461G, A461M, A461N, A461Y, T462C, T462E, T462F, T462L, S463K, S463Q, S463T, S463V, A464M, A464P, A464V, A464W, K465F, K465L, K465V, K465Y, L466A, L466E, L466F, L466G, L466M, L466P, L466Q, L466S, L466V, L466Y, V467D, V467E, V467G, V467T, V467W, A468D, A468E, A468F, A468K, A468L, A468V, A468W, W469A, W469G, W469M, T470M, K471F, K471Q, K471W, A472G, A472Y, D474R, D474W, S475E, S475F, R477L, P478G, P478L, P478V, M479G, M479I, M479R, M479W, T480C, T480G, K485E, K485R, K487A, K487F, K487G, K487N, K487S, K487W, A488C, A488G, A488H, A488N, A488S, N491A, N491W, E492A, E492W, S493E, S493G, S493H, S493L, S493M, S493Q, N494M, N494R, N494V, T495R, T495V, T495W, M496F, G497D, D498A, D498C, D498M, D498S, N499R, N499T, N499Y, L500A, L500N, T501G, T501M, A502L, N503A, N503E, N503M, G504H, G504K, G504P, G505A, G505D, G505E, G505H, G505L, G505N, G505R, G505S, V506C, V506D, V506E, V506G, V506L, V506P, V506R, V506S, V507A, V507G, V507L, V507N, V507R, V507S, G508C, G508E, T509A, T509I, T509K, T509M, T509V, T509Y, N510A, N510F, N510I, N510Q, Y511A, S512C, S512F, S512G, S512I, S512M, S512Q, S512T, S512V, S512Y, D513K, D513L, D513P, D513R, G514F, G514L, A515C, A515D, A515E, A515G, A515K, A515P, A515R, N516C, N516E, N516G, N516I, N516Q, N516T, N516V, Y517G, Y517N, D518Q, D518Y, K519C, K519E, K519G, K519I, K519Q, I520A, I520C, I520G, I520H, I520M, I520N, I520Q, I520S, I520T, I520W, R521N, R521V, T522G, T522N, H524R, H524V, P525G, P525T, S526G, W527A, W527E, W527G, W527H, W527N, W527R, W527S, A528C, A528E, A528I, I529G, Y530A, Y530M, G531E, G531S, G531T, T534A, T534I, A535I, A535M, A537D, A537M, A537P, A537S, I538M, N539W, S540E, S540G, G542Q, I543V, N545Q, N545S, R546C, R546L, R546P, R546S, T547A, T547D, T547N, T547S, T548E, T548F, T548K, T548L, T548P, G549D, G549F, G549P, G549W, G550S, A551I, A551Q, S553F, S553N, S553P, S554N, S554V, D555S, K556A, K556R, K556W, Q557R, L558E, L558H, L558P, T559Q, T559Y, Y561R, N563S, S564A, S564C, S564F, S564W, A565C, A565D, A565E, A565F, A565G, A565I, A565L, A565R, A565S, A565T, G567Q, G567V, A570G, A570K, A570M, A570W, A572S, A572W, S573G, S573K, S574K, S574Q, S574W, A575D, A575V, W576F, W576V, W576Y, Y577G, Y577L, D578M, D578N, V579E, V579G, V579L, V579T, V580A, V580D, V580K, V580L, V580S, Q581F, Q581G, Q581P, Q581S, Q581Y, R582A, R582G, R582L, R582Y, D583W, F584E, F584W, V585I, V585M, A586D, A586H, A586K, G587A, G587C, T588C, T588D, T588I, T588L, T588M, Y589I, Y589Q, Y589V, Y589W, V590A, V590H, W591F, T592C, T592L, T592S, G593I, F594L, D595E, D595Q, D595S, L597D, L597E, G598N, P600A, P600E, P600G, P600S, N604E, T606S, G607Q, G607S, S608T, S608V, G609A, G609L, G609N, G609R, A610F, A610M, A610T, V611K, G612N, G612T, W614A, W614P, P615L, S616W, N619A, N619I, N619L, S620G, I624A, V625E, V625M, V625Y, T627K, T627Q, A628D, A628N, G629T, F630C, F630D, F630G, F630Y, P631A, P631D, P631V, P631Y, K632D, K632G, K632T, T634A, T634E, T634V, Y635R, F637G, F637I, F637S, F637T, F637V, Y638A, Y638W, Q639R, D644C, D644Y, D645S, V646R, H647G, T648C, H650E, H650R, I651T, L652C, L652W, P653Q, A654K, A654M, A654R, W655R, N656V, E657R, V659N, A661E, A661H, A661K, A661M, A661Q, A661W, K662S, K662V, K662Y, N667L, N667R, P669L, P669T, P669W, V670C, Y673E, Y673G, Y673R, Y673S, T674C, T674M, D675A, D675P, D675Q, A676C, A676E, A676P, A677E, A677G, K678A, K678T, V679G, V679Q, V679S, V679Y, K680H, K680I, K680N, K680V, L681E, L681F, L681S, L681T, Y682E, F683M, F683Q, T684G, T684V, K686M, G687A, G687P, G687Q, G687R, S688E, S688K, S688L, S688T, T689D, T689G, T689P, T689W, E690D, E690V, K691P, K691R, R692G, R692H, R692I, R692S, R692T, I694L, I694W, G695C, G695L, G695R, G695W, E696A, E696R, K697A, K697E, K697G, K697R, S698E, S698I, S698L, S698M, S698T, T700C, K701A, K701D, K701G, K701L, K701M, K701S, T704M, T704Y, A705C, A705E, A705N, A705R, A705W, A706C, A706T, A706Y, Y708C, Y708F, Y708T, T709M, Y710D, Y710G, Y710N, Y710T, Y710V, Y710W, Q711A, Q711L, Q711M, Q711Y, V712M, V712P, V712Q, Y713G, E714H, E714K, E714V, G715K, A716C, A716L, A716R, A716T, A716V, D717C, D717G, D717S, K718A, K718L, K718Q, K718T, K718Y, D719E, D719T, D719V, S720A, T721H, T721L, T721N, T721S, T721V, T721W, A722T, A722V, H723G, H723P, M726D, M726V, Y727L, L728S, T729M, N731E, N731Q, N731S, V732R, V732W, P733R, P733W, W734G, W734V, A735Q, A735R, A735S, G737F, T738S, I739K, S740D, S740F, A741I, A741P, A741S, E742Q, Y744R, D745C, D745F, D745N, N747F, N747R, L750S, I751C, P752A, P752C, P752Y, G754H, G754P, G754R, T756N, T756S, E757A, N759S, N759V, A760N, G766M, A769F, A769G, A769I, A769N, A769V, A769Y, K770H, L771A, L771I, K772A, K772C, K772P, K772V, K772W, A773C, A773H, A773M, A773R, A773S, A773V, D774A, D774R, A775L, A775W, D776I, D776L, D776S, D776V, R777D, R777E, R777G, R777H, R777P, R777S, K778A, K778F, K778G, K778L, K778N, I780V, T781A, T781C, T781F, T781G, T781M, T781P, T781R, T781Y, A782E, A782K, A782P, A782Q, A782Y, D783A, D783C, D783E, D783R, G784A, G784L, G784S, G784T, K785C, K785S, K785W, K785Y, D786V, L787P, L787T, S788A, S788G, S788I, Y789V, I790A, I790C, I790F, I790R, E791S, E791T, V792G, V792S, D793H, D793K, V794C, V794T, V794W, T795P, D796Q, N798I, G799K, G799L, G799M, G799Q, H800A, H800L, H800V, I801C, V802I, V802S, V802Y, P803F, P803K, P803S, P803Y, D804G, D804K, D804S, A805F, A806I, A806Q, N807Q, N807W, R808C, R808F, R808G, R808I, R808N, R808Q, V809C, V809L, V809M, T810P, T810Q, T810Y, F811L, F811Y, D812Q, V813T, V813W, K814G, K814H, K814L, K814P, G815A, G815M, G815P, G815V, A816C, A816D, A816F, A816V, A816W, G817I, G817N, K818D, K818F, K818L, K818Q, K818W, K818Y, L819F, V820C, V820K, V820R, G821A, G821E, G821F, G821I, G821N, G821V, V822A, V822D, V822E, G825A, S826F, S826G, S826L, S826R, S827Q, P828C, P828Y, D829S, H830E, H830G, H830M, H830P, H830R, H830V, D831F, D831I, D831P, D831R, D831V, S832G, S832L, S832M, S832R, Y833E, Y833I, Y833K, Y833P, Y833V, Q834F, Q834M, A835D, A835E, A835H, A835K, A835W, D836C, D836E, D836H, D836Q, D836S, D836T, D836V, D836W, D836Y, N837D, N837G, N837H, N837L, R838G, R838M, R838N, R838W, K839A, K839D, K839E, A840G, A840I, A840P, A840V, F841C, F841W, S842M, G843C, K844A, K844G, V845N, V845W, L846I, L846M, V849A, V849L, V849S, Q850C, Q850G, Q850Y, K853N, K853P, K853Q, E854C, E854I, E857P, I858D, I858E, I858K, I858M, I858P, I858Q, I858Y, T859V, V860T, T861F, T861I, T861W, A862V, K863F, K863I, K863N, K863W, A864L, D865A, D865G, G866H, G866R, L867W, S870E, S870M, S870R, S870T, T871P, V872C, V872G, K873G, K873Y, I874G, T877A, P880S, G881W, T882A, T882R, S883L, T884A, E885V, K886E, K886L, K886V, K886W, T887A, T887D, T887F, T887G, T887N, T887V, V888A, V888D, R889G, Y892D, Y892P, Y892R, Y893E, S894D, N896M, Y897V, V899G, K900E, T901G, T901Q, T901R, T901V, T901Y, G902A, G902F, G902L, G902P, G902Q, G902R, G902S, G902W, N903D, N903I, K904D, K904E, K904M, K904N, K904R, K904S, K904V, P905C, P905R, P905W, P905Y, I906N, I906S, I906W, I906Y, L907F, L907S, P908D, P908G, P908I, P908L, D910W, E912T, V913Q, R914A, R914F, R914I, R914K, R914V, R914W, R914Y, Y915A, Y915C, Y915G, Y915I, Y915M, Y915Q, Y915V, S916G, D917C, D917E, D917F, D917L, D917M, D917Q, D917S, G918E, G918H, G918T, T919D, T919Q, T919W, T919Y, S920E, S920W, D921Q, D921V, R922A, R922G, R922M, R922W, Q923A, Q923V, N924L, V925A, V925C, V925K, V925S, T926G, T926R, T926S, W927C, W927G, W927P, D928A, D928Q, A929C, A929P, V930A, V930I, S931G, S931R, D932F, D932S, D932T, D932V, D933I, D933R, Q934S, Q934V, I935A, I935C, I935E, I935P, I935W, A936I, A936Q, A936Y, K937G, K937I, K937M, K937P, K937Q, K937R, A938C, A938T, G939D, S940C, S940E, S940R, S940T, S940V, S940W, F941C, F941W, S942K, S942L, S942V, V943A, V943H, V943R, A944D, A944H, A944P, A944R, G945T, T946A, T946G, T946P, T946V, T946W, V947G, V947L, V947M, V947P, V947R, V947T, A948C, A948I, A948R, A948W, G949A, Q950D, Q950G, K951D, K951Q, K951S, K951W, K951Y, I952H, S953F, S953M, S953R, S953W, V954D, V954Q, V954T, R955C, R955W, V956D, V956Q, T957S, M958D, M958I, I959V, I959Y, D960H, D960L, D960S, E961D, E961F, E961S, E961T, I962A, I962C, I962K, I962N, L965C, L965G, L965K, L965M, L965P, L965V, L965Y, L966A, L966G, L966H, L966N, L966P, L966T, N967D, N967M, N967P, N967S, N967T, Y968G, Y968L, Y968Q, Y968V, S969C, S969D, S969H, S969I, S969L, S969M, S969Q, S969Y, A970I, S971H, S971V, S971W, P973C, P973D, P973N, P973R, P973W, V974E, V974T, V974Y, G975Q, T976F, T976G, T976K, T976P, P977C, P977T, P977Y, A978F, A978M, A978N, A978R, V979R, L980A, L980H, L980I, L980N, L980Q, L980T, P981M, G982P, G982Q, P985E, P985L, P985W, A986I, A986L, A986N, A986W, V987A, V987F, V987K, L988E, L988G, L988S, P989A, P989M, D990W, G991F, G991H, G991Y, T992M, T994S, S995L, S995V, A996Q, A996V, N997A, N997E, N997K, N997L, N997Y, F998M, F998W, A999G, A999M, A999R, A999S, V1000C, V1000L, V1000M, V1000N, D1001G, D1001K, D1001L, D1001T, D1001V, W1002A, W1002D, W1002E, W1002H, W1002N, W1002P, W1002Q, T1003L, T1003P, T1003Y, K1004F, K1004H, K1004P, K1004R, K1004V, P1005I, P1005Y, A1006C, A1006I, A1006S, D1007L, D1007P, D1007V, V1009G, Y1010T, N1011A, N1011T, T1012Q, A1013Q, A1013V, G1014I, G1014L, T1015G, V1016C, V1016D, V1018K, T1021F, T1021G, T1021V, A1022H, A1022L, T1023D, V1024G, V1024H, V1024K, V1024N, V1024R, V1024S, V1024W, G1026L, G1026R, G1026S, G1026V, K1027C, K1027N, F1029K, F1029P, F1029V, K1030D, K1030W, V1031H, V1031K, T1034H, I1035D, R1036G, R1036L, R1036T, V1037F, Q1041P, V1042N, T1043F, I1044A, S1046M, V1048C, V1048F, V1048G, V1048M, V1048Q, G1050S, N1051E, N1051K, A1052K, L1053A, L1053W, L1055R, Q1057E, Q1057R, N1058R, N1058S, N1058V, I1059W, P1060G, P1060N, P1060T, A1061G, D1062A, D1062L, D1062M, K1063D, Q1064M, Q1064R, S1065A, S1065C, S1065E, D1066A, D1066G, D1066M, D1066V, D1066W, T1067M, L1068C, L1068P, L1068Q, A1070P, I1071R, I1071W, K1072E, K1072G, K1072Q, K1072S, D1073L, D1073P, G1074I, G1074L, G1074R, S1075C, S1075L, T1076H, T1076Q, T1076S, V1078L, T1082E, T1082F, G1083E, G1083F, G1083L, G1083S, G1084V, G1084Y, G1085S, A1086H, A1086Q, N1087R, N1087W, P1088E, P1088R, S1089C, S1089G, S1089K, S1089Q, S1089R, A1090G, W1091A, W1091E, W1091G, W1091H, W1091T, W1091V, W1091Y, T1092G, T1092Q, T1092S, T1092V, N1093A, N1093P, N1093Q, N1093T, N1093V, W1094D, W1094E, W1094P, A1095W, Y1096A, Y1096D, S1097L, S1097T, S1097W, K1098D, K1098F, A1099C, A1099D, A1099S, A1099V, G1100H, G1100M, G1100T, H1101V, N1102E, N1102H, N1102K, T1103E, A1104R, E1105L, I1106T, T1107R, T1107S, F1108D, F1108L, F1108T, E1109A, E1109D, E1109L, E1109W, A1111G, E1113D, E1113P, E1113V, Q1114E, Q1114R, Q1114S, Q1114T, Q1114V, Q1115A, Q1115K, Q1115L, Q1115P, L1116G, L1116K, L1116W, G1117E, G1117I, G1117M, G1117R, G1117S, G1117T, G1117V, G1117W, Q1118W, I1119E, I1119G, I1119N, I1119S, V1120T, M1121G, M1121K, M1121V, Y1122K, Y1122R, Y1122V, F1123R, F1123T, F1124V, F1124W, R1125D, R1125E, R1125K, R1125V, R1125W, D1126H, D1126L, S1127F, S1127I, S1127K, S1127Q, S1127W, N1128A, N1128R, N1128S, N1128W, A1129E, A1129L, A1129Q, A1129V, V1130A, V1130G, V1130R, V1130S, R1131A, R1131S, F1132E, P1133D, P1133G, P1133R, D1134G, D1134L, A1135L, A1135S, A1135W, A1135Y, G1136A, G1136E, G1136P, K1137C, K1137L, K1137P, K1137Q, K1137R, K1137S, T1138R, I1140C, I1140G, I1140L, I1140P, Q1141K, Q1141P, Q1141W, I1142Y, S1143G, A1144C, A1144N, A1144P, A1144S, A1144T, A1144V, A1144W, D1145E, D1145R, D1145T, G1146D, G1146L, G1146V, K1147T, K1147V, N1148P, W1149C, W1149G, W1149I, W1149N, W1149Q, W1149S, W1149V, T1150K, D1151R, D1151W, L1152A, L1152E, L1152Q, L1152W, A1153G, A1154C, A1154R, T1155L, I1158R, I1158W, A1159I, A1159R, A1159V, A1160Q, Q1161A, Q1161P, Q1161S, E1162A, E1162C, E1162I, E1162Q, E1162Y, E1165D, E1165L, E1165S, V1167R, K1168Q, K1168W, P1169S, Y1170E, Y1170K, T1171A, T1171G, Y1172E, Y1172K, D1173A, D1173F, D1173K, D1173L, F1174P, F1174T, F1174V, F1174W, A1175N, A1175Q, A1175S, A1175V, A1175Y, V1177N, V1177T, G1178M, G1178Q, G1178T, A1179L, A1179W, T1180A, T1180G, T1180I, T1180M, T1180Q, T1180Y, V1182M, K1183E, V1184C, V1184E, V1184K, V1184L, V1184P, V1184Q, V1184Y, V1186S, N1188W, A1189C, A1189K, A1189T, A1189V, D1190R, D1190T, T1191E, T1191L, T1192H, V1197A, V1198E, C1199T, A1200G, A1200V, A1200W, L1202A, T1203K, E1204G, I1205V, K1208R, K1208V, K1208W, T1209A, T1209C, T1209E, T1209K, T1209N, T1209Q, A1210D, A1210E, A1210G, A1210K, A1210L, A1210R, A1210T, A1210W, T1211D, T1211G, T1211K, T1211P, T1211R, T1211S, K1213T, F1214A, F1214E, F1214K, F1214L, F1214P, F1214S, F1214V, V1215D, V1215L, V1215Q, T1216A, T1216L, T1216P, T1216Q, N1217A, N1217F, N1217P, N1217R, N1217S, N1217T, T1218A, T1218C, T1218E, T1218G, T1218S, T1218W, S1219A, S1219F, S1219I, S1219R, A1220L, A1220P, A1220R, A1221D, A1221R, A1221V, A1221W, L1222A, L1222E, L1222F, L1222V, L1222W, S1223F, S1223K, S1223L, S1223V, S1224A, S1224D, S1224G, S1224P, S1224W, L1225C, L1225E, L1225F, L1225K, L1225W, T1226A, T1226G, T1226P, T1226S, T1226V, V1227A, V1227D, V1227L, V1227P, V1227S, N1228D, N1228F, N1228T, G1229V, T1230H, T1230I, T1230K, T1230S, T1230W, K1231F, K1231L, K1231M, K1231P, V1232K, V1232R, S1233P, S1233W, D1234K, D1234R, S1235E, S1235G, S1235R, S1235W, V1236A, V1236G, V1236I, V1236P, V1236Q, L1237D, L1237E, A1238E, A1238L, A1238N, A1238P, A1238R, A1239D, A1239P, A1239R, G1240D, G1240N, G1240W, S1241D, Y1242E, Y1242R, N1243M, N1243P, N1243T, N1243V, N1243W, T1244A, T1244E, T1244Q, T1244W, A1246M, A1246N, A1246P, A1246Q, A1246R, I1247G, I1247Q, I1247R, I1247S, I1247W, I1248G, I1248L, I1248R, I1248S, A1249G, A1249H, A1249I, A1249R, A1249T, A1249V, D1250I, D1250K, D1250S, D1250T, D1250W, D1250Y, V1251T, V1251W, K1252D, K1252V, K1252W, E1254V, G1255H, G1255M, E1256M, E1256N, E1256V, E1256W, G1257F, G1257L, G1257Q, N1258H, N1258S, A1259K, V1261I, V1261L, T1262A, T1262Q, T1262R, V1263E, V1263R, V1263T, V1263W, L1264H, P1265L, P1265R, P1265W, A1266P, A1266V, H1267E, N1269A, N1269K, N1269R, N1269S, V1270D, I1271H, R1272M, R1272V, V1273R, I1274F, I1274R, T1275L, T1275W, S1277L, S1277T, S1277W, E1278Q, D1279G, D1279I, D1279R, D1279T, D1279W, H1280E, H1280G, V1281I, V1281W, T1282D, R1283A, R1283D, R1283E, R1283W, K1284G, T1285F, F1286A, F1286E, F1286P, F1286S, T1287C, T1287R, T1287S, T1287W, I1288A, I1288D, I1288G, I1288K, N1289A, N1289T, L1290A, L1290R, G1291P, G1291V, G1291Y, T1292Y, E1293K, E1293L, E1293S, E1293V, Q1294E, Q1294P, Q1294W, E1295K, F1296A, P1297F, E1302R, E1302S, R1303S, D1304A or D1304V.

83. The lactase variant of the preceding embodiment, wherein the variant has a decreased or an increased galactose inhibition, preferably measured as a galactose inhibition improvement factor (GI-IF) of at least 1.2 or at most 0.8, compared to the lactase of SEQ ID NO: 1.

84. The lactase variant of embodiment 1 which comprises one or more of the following substitutions, wherein each position corresponds to a position in SEQ ID NO: 1: V1D, V1F, V1H, V1L, E2D, E2Q, E2V, D3I, D3S, D3V, D3W, T5D, T5S, R6A, R6F, R6M, R6P, S7D, S7I, S7N, S7P, S9H, S9P, T10L, T10P, T10S, T11L, T11P, Q12R, M13C, M13D, M13F, M13K, M13R, S14V, S15C, S15I, S15K, S15P, S15R, S15T, S15V, S15Y, T16C, P17D, P17I, P17L, P17T, V19G, V19K, V20C, V20G, V20K, V20L, V20M, V20P, V20Q, V20R, V20W, Y21C, Y21P, Y21R, Y21T, S22E, S22G, S22N, S22T, S22W, S23C, S23L, S23M, S23R, A24F, A24L, A24R, A24T, A24W, V25D, V25K, V25M, V25Q, V25S, D26C, D26I, D26L, S27A, S27C, S27F, S27G, S27H, S27Y, K28C, K28L, K28V, Q29G, Q29L, Q29M, Q29R, Q29V, Q29W, N30A, N30M, N30V, R31E, R31G, R31I, R31M, R31V, T32S, S33C, S33N, S33Q, S33R, D34C, D34E, D34G, D34H, D34L, D34S, D34W, D34Y, F35C, F35E, F35K, F35N, D36Q, N38S, W39G, W39S, K40C, K40D, K40F, K40G, K40I, K40N, K40W, F41A, F41C, F41G, F41I, F41Q, M42E, M42N, M42T, L43A, L43C, L43I, L43S, L43T, L43V, S44C, D45A, D45P, V47K, V47R, A49D, A49H, A49R, A49S, A49T, A49V, D51G, D51I, D51K, D51P, D51V, A53C, A53R, A53S, A53V, A53W, D55C, D55F, D55G, D55H, D55M, D55P, D55S, S57A, S57C, S57E, S57G, A58D, A58G, A58Q, A58T, W59I, W59K, W59P, W59V, Q60K, Q60M, Q60R, Q60S, Q60Y, Q61P, V62N, V62S, V62T, V62W, D63G, L64E, L64G, H66L, H66R, H66Y, Y68P, I70A, I70K, T71C, T71K, T71Q, K73D, Y74K, S75G, S75L, S75Q, S75R, S75V, Q76G, Q76I, Q76K, Q76V, S77C, S77D, S77E, S77F, S77G, S77H, S77K, S77L, S77M, S77R, S77T, S77V, N78C, N78E, N78K, N78Q, N78R, N78S, E79H, E79Q, E79S, E79T, A80K, E81A, E81Q, A83T, L85A, L85F, L85N, L85S, L85V, L85W, P86G, P86N, P86Q, P86R, P86V, P86W, P86Y, G87D, G87N, G87Q, G88F, G88Q, G88S, T89H, T89K, T89M, T89Y, G90A, G90L, G90S, G90T, G90V, W91E, W91L, W91P, W91Q, W91R, W91Y, Y92F, Y92H, Y92I, Y92M, Y92N, Y92S, Y92T, Y92V, R93A, R93D, R93F, R93H, R93I, R93L, R93N, R93T, R93V, R93Y, K94A, K94C, K94G, K94S, K94T, K94V, S95A, S95C, S95D, S95G, S95I, S95Q, S95R, F96A, F96C, F96I, F96K, F96M, F96P, F96S, F96V, F96W, T97F, T97V, I98C, I98H, I98W, R100T, D101A, D101P, D101V, L102P, R106V, R106W, I107F, I107G, I107S, A108E, A108S, I109M, N110A, N110S, N110T, N110V, N110W, F111A, F111L, F111Q, D112F, D112T, G113S, V114F, V114R, Y115E, M116C, N117W, A118K, A118P, A118Y, T119A, V120A, V120K, W121D, W121R, W121V, W121Y, F122A, F122S, F122Y, N123P, G124E, G124M, G124Q, G124R, V125E, V125I, G128D, T129E, H130A, H130C, H130S, H130T, Y132C, Y132E, P136R, P136Y, F137C, F137L, S138A, S138L, S138R, F139Q, D140V, L141G, T142S, T142V, K146A, G148T, G149M, G149Q, G149Y, E150C, E150G, E150L, E150R, I153A, V154I, V154K, V154L, V155F, V157L, V157P, V157S, E158G, E158H, E158V, N159H, N159S, N159T, L161K, L161M, L161S, L161W, P162N, P162T, P162W, S164A, R165H, W166S, Y167A, Y167C, G169D, S170L, S170Q, G171C, G171T, I172K, I172P, Y173H, Y173M, Y173W, R174E, R174K, V176E, V176K, V176T, T177K, L178I, L178Q, L178W, T179A, T179C, T179D, T179I, T179K, T179N, T179P, T179S, V180A, V180D, V180G, T181A, T181F, D182F, G183W, V184F, V184P, V184R, V184S, V184W, H185G, H185L, V186E, V186N, G187D, G187H, N188E, N188R, N188V, N189E, G190C, G190F, I193N, I193Q, K194I, T195S, T195W, P196A, P196I, S197C, S197E, S197L, L198R, L198V, A199E, A199K, A199P, Q201E, N202D, N202L, N202Q, N202R, N202S, N202T, N202W, G203C, G203K, G203S, G203V, G203W, G203Y, G204A, G204K, G204Y, N205E, N205G, N205H, N205L, V206I, V206K, V206S, T207A, T207C, T207G, T207I, T207K, T207Q, M208S, N209D, N209K, N209V, L210G, L210I, L210Q, L210V, T211A, T211F, T211Q, T211R, T212E, T212G, T212H, T212L, T212S, K213C, K213D, K213F, K213L, K213T, K213Y, V214A, V214C, V214W, A215D, A215E, A215I, A215K, A215L, A215Q, A215R, A215V, D217F, D217L, D217M, D217T, T218G, K219F, K219H, K219M, A220C, A220G, A220L, A220V, A221E, A221L, A221R, A222D, A222I, A222L, A222P, A222R, A222W, N223E, N223F, N223K, N223L, N223S, N223T, L226M, L226Q, Q228N, Q228R, T229A, T229C, T229D, T229G, T229M, T229N, T229Q, T229R, T229V, V230L, V230M, V230R, V230S, F231E, F231G, F231I, F231L, F231V, F231W, F231Y, P232G, P232M, P232S, K233C, K233E, K233F, K233L, K233P, K233R, G234A, G234C, G234D, G234E, G234L, G234Q, G234R, G234V, G234Y, G235H, G235I, G235K, G235M, G235Q, G235T, G235W, G235Y, K236A, K236D, K236L, K236M, K236P, K236R, K236S, K236W, K236Y, T237D, T237K, T237R, D238A, D238E, D238F, D238G, D238M, D238P, D238R, A239E, A239G, A239I, A239T, A240C, A240L, A240P, A240T, A240Y, I241T, G242K, G242L, G242M, G242P, G242Y, T243I, T243M, T243R, V244E, V244R, T245E, T245G, T245Q, T245R, T246G, T246K, A247E, A247K, A247P, A247R, A247S, A247V, A247W, S248A, S248E, S248L, S248Q, S248T, K249A, K249D, K249G, K249H, K249L, K249N, K249P, K249Q, K249V, K249Y, S250H, I251L, I251W, I251Y, A252F, A252W, A252Y, G254D, G254F, G254I, G254L, G254M, G254Q, G254R, G254W, A255K, A255S, A255Y, S256A, S256C, S256K, S256M, S256N, S256R, S256W, S256Y, A257D, A257I, A257N, A257V, D258A, D258L, D258W, V259E, V259T, T260D, T260G, S261D, S261H, S261R, S261W, T262D, T262W, T264F, T264M, T264Q, T264Y, A265I, A265S, A266D, A266G, A266K, A266Q, S267A, S267M, S267N, S267P, S267R, S267V, P268F, P268G, P268R, P268W, P268Y, K269V, K269Y, L270D, L270N, S272G, S272T, S272W, I273L, I273R, N275W, N277F, N277R, L278H, L278I, L278K, L278M, L278Q, L278R, L278V, Y279W, T280A, T280E, T280F, T280Q, V281I, V281Q, R282E, R282F, R282H, R282I, R282K, R282N, R282S, R282V, R282W, T283R, T283V, E284A, E284D, E284H, E284L, E284R, E284Y, V285T, L286F, L286N, L286T, L286W, G288S, V291D, V291F, V291P, V291T, V291Y, L292D, L292H, L292V, D293C, D293I, D293S, D293W, T294Q, T294S, Y295F, Y295W, D296F, D296H, D296K, D296R, D296V, T297I, E298I, E298L, E298R, F301C, R302I, R302N, W303A, W303C, W303D, W303F, W303S, T304D, T304E, T304I, T304K, T304P, G305L, G305N, G305P, G305T, G305W, F306K, F306L, D307A, D307E, D307G, D307S, D307W, A308E, A308M, A308Y, T309C, T309D, T309E, T309I, T309S, T309V, S310A, S310H, S310L, S310M, S310N, S310V, G311V, F312H, F312R, L314A, L314C, L314T, L314V, N315S, G316R, G316S, E317K, E317W, K318C, K318N, K320C, K320E, K320F, K320I, K320L, K320R, K320T, K320V, K320W, L321M, K322F, K322I, K322P, K322S, V324T, S325D, S325G, S325T, M326E, M326G, M326T, M326Y, H328C, H328F, H328G, H328L, H328M, H328R, H328T, D329S, G331V, A335C, A335G, A335L, A337E, A337G, A337L, N338P, R340A, R340C, R340L, A341M, A341S, I342G, I342K, I342P, E343N, E343T, E343Y, R344G, Q345F, Q345G, Q345K, Q345N, Q345S, V346A, V346C, V346I, V346S, E347A, E347C, E347F, E347I, E347R, I348D, I348M, I348Q, I348R, L349A, L349Q, L349S, Q350G, Q350N, K351V, M352G, M352T, M352W, G353K, V354E, V354S, V354W, N355M, N355W, I357T, T359E, T359L, T360V, N362S, N362T, P363A, P363D, P363I, P363L, P363M, P363S, P363V, A364C, A364E, A364F, A364G, A364I, A364M, A364P, A364V, A365C, A365E, A365I, A365P, A365V, A365W, K366A, K366D, K366L, K366M, K366P, K366S, K366V, A367Q, L368A, L368E, L368Q, L368S, L368Y, I369A, I369D, I369E, I369G, I369K, I369M, I369R, I369V, I369W, D370L, D370Q, D370S, V371D, V371F, V371L, V371Q, V371S, C372P, N373G, N373L, E374R, K375D, K375S, G376A, G376S, V377A, V377M, V377T, V379A, V379N, V380P, E381A, E381G, E381Q, E381T, V383A, V383K, V383L, M386G, M386N, M386S, M386V, W387H, W387L, N388A, N388E, N388L, N388R, R389A, R389C, R389E, R389N, R389Q, R389S, R389T, S390C, S390D, S390G, S390P, S390Q, S390T, S390V, K391E, N392D, G393A, G393E, G393N, G393R, G393S, G393V, N394L, T395C, T395F, T395H, T395M, T395N, T395S, T395W, E396K, E396L, E396M, E396V, E396W, Y398M, Y398N, G399S, K400A, K400C, K400N, K400P, K400Q, K400S, K400T, K400V, W401F, W401H, W401K, W401L, F402W, F402Y, G403A, G403D, G403K, G403P, G403Q, G403S, G403V, G403Y, Q404F, Q404H, Q404L, Q404M, Q404P, Q404R, Q404S, Q404V, A405C, A405E, A405H, A405K, A405P, A405R, I406C, I406D, I406N, A407C, A407G, A407Q, A407T, G408I, G408M, G408N, G408W, D409N, N410R, N410Y, A411E, A411R, A411S, A411V, L413I, L413P, L413T, G414A, G414C, G414M, G414N, G414R, G415Q, G415R, D416I, D416R, D416Y, K417C, K417F, K417G, K417R, K417T, D418P, D418R, E419M, E419R, E419W, T420E, T420F, T420G, T420K, T420R, T420V, W421L, W421Q, A422P, A422T, K423D, F424C, F424L, F424N, L426C, L426M, L426Q, T427D, T427F, T427G, T427K, T427M, T427P, T427Q, T427S, T427W, S428F, S428K, T429D, I430C, I430L, I430M, I430Q, I430S, I430T, N431D, N431E, N431M, N431Y, R432A, R432E, R432N, R432Q, R432Y, D433G, D433I, D433P, D433Q, D433W, R434N, R434S, R434V, N435E, N435F, N435K, N435L, N435R, N435V, N435W, A436G, A436L, P437A, P437Q, P437R, P437V, V439C, V439E, V439G, V439I, V439K, V439T, V439Y, I440F, I440K, I440R, I440W, M441A, M441E, M441Q, W442E, W442G, W442P, W442R, S443C, S443G, S443M, S443Q, S443Y, L444D, L444E, L444F, L444G, L444K, L444Q, L444V, L444W, G445A, G445C, G445V, N446D, N446T, M448A, M448C, M448D, M448I, M448L, M448P, M448Q, M448S, M448V, M449D, M449E, M449T, M449W, M449Y, E450F, E450S, E450T, E450V, E450W, G451C, G451F, G451L, G451P, G451V, G451W, I452G, I452K, I452L, I452M, I452Q, I452S, I452V, S453F, S453H, S453L, S453N, S453Q, G454L, G454W, S455E, S455K, S455M, S455P, S455R, S455W, V456D, V456E, V456F, V456K, V456L, S457H, S457K, S457P, S457Q, S457T, S457V, G458A, G458P, G458S, G458V, G458W, F459C, F459N, F459R, F459S, P460Y, A461D, A461G, A461M, A461N, A461Y, T462C, T462E, T462F, T462L, S463K, S463Q, S463T, S463V, A464M, A464P, A464V, A464W, K465F, K465L, K465V, K465Y, L466A, L466E, L466F, L466G, L466M, L466P, L466Q, L466S, L466V, L466Y, V467D, V467E, V467G, V467T, V467W, A468D, A468E, A468F, A468K, A468L, A468V, A468W, W469A, W469G, W469M, T470M, K471F, K471Q, K471W, A472G, A472Y, D474R, D474W, S475E, S475F, R477L, P478G, P478L, P478V, M479G, M479I, M479R, M479W, T480C, T480G, K485E, K485R, K487A, K487F, K487G, K487N, K487S, K487W, A488C, A488G, A488H, A488N, A488S, N491A, N491W, E492A, E492W, S493E, S493G, S493H, S493L, S493M, S493Q, N494M, N494R, N494V, T495R, T495V, T495W, M496F, G497D, D498A, D498C, D498M, D498S, N499R, N499T, N499Y, L500A, L500N, T501G, T501M, A502L, N503A, N503E, N503M, G504H, G504K, G504P, G505A, G505D, G505E, G505H, G505L, G505N, G505R, G505S, V506C, V506D, V506E, V506G, V506L, V506P, V506R, V506S, V507A, V507G, V507L, V507N, V507R, V507S, G508C, G508E, T509A, T509I, T509K, T509M, T509V, T509Y, N510A, N510F, N510I, N510Q, Y511A, S512C, S512F, S512G, S512I, S512M, S512Q, S512T, S512V, S512Y, D513K, D513L, D513P, D513R, G514F, G514L, A515C, A515D, A515E, A515G, A515K, A515R, N516C, N516E, N516G, N516I, N516Q, N516T, N516V, Y517G, Y517N, D518Q, D518Y, K519C, K519E, K519G, K519I, K519Q, I520A, I520C, I520G, I520H, I520M, I520N, I520Q, I520S, I520T, I520W, R521N, R521V, T522G, T522N, H524R, H524V, P525G, P525T, S526G, W527A, W527E, W527G, W527H, W527N, W527R, W527S, A528C, A528E, A528I, I529G, Y530A, Y530M, G531E, G531S, G531T, T534A, T534I, A535I, A535M, A537D, A537M, A537P, A537S, I538M, N539W, S540E, S540G, G542Q, I543V, N545Q, N545S, R546C, R546L, R546P, R546S, T547A, T547D, T547N, T547S, T548E, T548F, T548K, T548L, T548P, G549D, G549F, G549P, G549W, G550S, A551I, A551Q, S553F, S553N, S553P, S554N, S554V, D555S, K556A, K556R, K556W, Q557R, L558E, L558H, L558P, T559Q, T559Y, Y561R, N563S, S564A, S564C, S564F, S564W, A565C, A565D, A565E, A565F, A565G, A565I, A565L, A565R, A565S, A565T, G567Q, G567V, A570G, A570K, A570M, A570W, A572S, A572W, S573G, S573K, S574K, S574Q, S574W, A575D, A575V, W576F, W576V, W576Y, Y577G, Y577L, D578M, D578N, V579E, V579G, V579L, V579T, V580A, V580D, V580K, V580L, V580S, Q581F, Q581G, Q581P, Q581S, Q581Y, R582A, R582G, R582L, R582Y, D583W, F584E, F584W, V585I, V585M, A586D, A586H, A586K, G587A, G587C, T588C, T588D, T588I, T588L, T588M, Y589I, Y589Q, Y589V, Y589W, V590A, V590H, W591F, T592C, T592L, T592S, G593I, F594L, D595E, D595Q, D595S, L597D, L597E, G598N, P600A, P600E, P600G, P600S, N604E, T606S, G607Q, G607S, S608T, S608V, G609A, G609L, G609N, G609R, A610F, A610M, A610T, V611K, G612N, G612T, W614A, W614P, P615L, S616W, N619A, N619I, N619L, S620G, I624A, V625E, V625M, V625Y, T627K, T627Q, A628D, A628N, G629T, F630C, F630D, F630G, F630Y, P631A, P631D, P631V, P631Y, K632D, K632G, K632T, T634A, T634E, T634V, Y635R, F637G, F637I, F637S, F637T, F637V, Y638A, Y638W, Q639R, D644C, D644Y, D645S, V646R, H647G, T648C, H650E, H650R, I651T, L652C, L652W, P653Q, A654K, A654M, A654R, W655R, N656V, E657R, V659N, A661E, A661H, A661K, A661M, A661Q, A661W, K662S, K662V, K662Y, N667L, N667R, P669L, P669T, P669W, V670C, Y673E, Y673G, Y673R, Y673S, T674C, T674M, D675A, D675P, D675Q, A676C, A676E, A676P, A677E, A677G, K678A, K678T, V679G, V679Q, V679S, V679Y, K680H, K680I, K680N, K680V, L681E, L681F, L681S, L681T, Y682E, F683M, F683Q, T684G, T684V, K686M, G687A, G687P, G687Q, G687R, S688E, S688K, S688L, S688T, T689D, T689G, T689P, T689W, E690D, E690V, K691P, K691R, R692G, R692H, R692I, R692S, R692T, I694L, I694W, G695C, G695L, G695R, G695W, E696A, E696R, K697A, K697E, K697G, K697R, S698E, S698I, S698L, S698M, S698T, T700C, K701A, K701D, K701G, K701L, K701M, K701S, T704M, T704Y, A705C, A705E, A705N, A705R, A705W, A706C, A706T, A706Y, Y708C, Y708F, Y708T, T709M, Y710D, Y710G, Y710N, Y710T, Y710V, Y710W, Q711A, Q711L, Q711M, Q711Y, V712M, V712P, V712Q, Y713G, E714H, E714K, E714V, G715K, A716C, A716L, A716R, A716V, D717C, D717G, D717S, K718A, K718L, K718Q, K718T, K718Y, D719E, D719T, D719V, S720A, T721H, T721L, T721N, T721S, T721V, T721W, A722T, H723G, H723P, M726D, M726V, Y727L, L728S, T729M, N731E, N731Q, N731S, V732R, V732W, P733R, P733W, W734G, W734V, A735Q, A735R, A735S, G737F, T738S, I739K, S740D, S740F, A741I, A741P, A741S, E742Q, Y744R, D745C, D745F, D745N, N747F, N747R, L750S, I751C, P752A, P752C, P752Y, G754H, G754P, T756N, T756S, E757A, N759S, N759V, A760N, G766M, A769F, A769G, A769I, A769N, A769V, A769Y, K770H, L771A, L771I, K772A, K772C, K772P, K772V, K772W, A773C, A773H, A773M, A773R, A773S, A773V, D774A, D774R, A775L, A775W, D776I, D776L, D776S, D776V, R777D, R777E, R777G, R777H, R777P, R777S, K778A, K778F, K778G, K778L, K778N, I780V, T781A, T781C, T781F, T781G, T781M, T781P, T781R, T781Y, A782E, A782K, A782P, A782Q, A782Y, D783A, D783C, D783E, D783R, G784A, G784L, G784S, G784T, K785C, K785S, K785W, K785Y, D786V, L787P, L787T, S788A, S788G, S788I, Y789V, I790A, I790C, I790F, I790R, E791S, E791T, V792G, V792S, D793H, D793K, V794C, V794T, V794W, T795P, D796Q, N798I, G799K, G799L, G799M, G799Q, H800A, H800L, H800V, I801C, V802I, V802S, V802Y, P803F, P803K, P803S, P803Y, D804G, D804K, D804S, A805F, A806I, A806Q, N807Q, N807W, R808C, R808F, R808G, R808I, R808N, R808Q, V809C, V809L, V809M, T810P, T810Q, T810Y, F811L, F811Y, D812Q, V813T, V813W, K814G, K814H, K814L, K814P, G815A, G815M, G815P, G815V, A816C, A816D, A816F, A816V, A816W, G817I, G817N, K818D, K818F, K818L, K818Q, K818W, K818Y, L819F, V820C, V820K, V820R, G821A, G821E, G821F, G821I, G821N, G821V, V822A, V822D, V822E, G825A, S826F, S826G, S826L, S826R, S827Q, P828C, P828Y, D829S, H830E, H830G, H830M, H830P, H830R, H830V, D831F, D831I, D831P, D831R, D831V, S832G, S832L, S832M, S832R, Y833E, Y833I, Y833K, Y833P, Y833V, Q834F, Q834M, A835D, A835E, A835H, A835K, A835W, D836C, D836E, D836H, D836Q, D836S, D836T, D836V, D836W, D836Y, N837D, N837G, N837H, N837L, R838G, R838M, R838N, R838W, K839A, K839D, K839E, A840G, A840I, A840P, A840V, F841C, F841W, S842M, G843C, K844A, K844G, V845N, V845W, L846I, L846M, V849A, V849L, V849S, Q850C, Q850G, Q850Y, K853N, K853P, K853Q, E854C, E854I, E857P, I858D, I858E, I858K, I858M, I858P, I858Q, I858Y, T859V, V860T, T861F, T861I, T861W, A862V, K863F, K863I, K863N, K863W, A864L, D865A, D865G, G866H, G866R, L867W, S870E, S870M, S870R, S870T, T871P, V872C, V872G, K873G, K873Y, I874G, T877A, P880S, G881W, T882A, T882R, S883L, T884A, E885V, K886E, K886L, K886V, K886W, T887A, T887D, T887F, T887G, T887N, T887V, V888A, V888D, R889G, Y892D, Y892P, Y892R, Y893E, S894D, N896M, Y897V, V899G, K900E, T901G, T901Q, T901R, T901V, T901Y, G902A, G902F, G902L, G902P, G902Q, G902R, G902S, G902W, N903D, N903I, K904D, K904E, K904M, K904N, K904R, K904S, K904V, P905C, P905R, P905W, P905Y, I906S, I906W, I906Y, L907F, L907S, P908D, P908G, P908I, P908L, D910W, E912T, V913Q, R914A, R914F, R914I, R914K, R914V, R914W, R914Y, Y915A, Y915C, Y915G, Y915I, Y915M, Y915Q, Y915V, S916G, D917C, D917E, D917F, D917L, D917M, D917Q, D917S, G918E, G918H, G918T, T919D, T919Q, T919W, T919Y, S920E, S920W, D921Q, D921V, R922A, R922G, R922M, R922W, Q923A, Q923V, N924L, V925A, V925C, V925K, V925S, T926G, T926R, T926S, W927C, W927G, W927P, D928A, D928Q, A929C, A929P, V930A, V930I, S931G, S931R, D932F, D932S, D932T, D932V, D933I, D933R, Q934S, Q934V, I935A, I935C, I935E, I935P, I935W, A936I, A936Q, A936Y, K937G, K937I, K937M, K937P, K937Q, K937R, A938C, A938T, G939D, S940C, S940E, S940R, S940T, S940V, S940W, F941C, F941W, S942K, S942L, S942V, V943A, V943H, V943R, A944D, A944H, A944P, A944R, G945T, T946A, T946G, T946P, T946V, T946W, V947G, V947L, V947M, V947P, V947R, V947T, A948C, A948I, A948R, A948W, G949A, Q950D, Q950G, K951D, K951Q, K951S, K951W, K951Y, I952H, S953F, S953M, S953R, S953W, V954D, V954Q, V954T, R955C, R955W, V956D, V956Q, T957S, M958D, M958I, I959V, I959Y, D960H, D960L, D960S, E961D, E961F, E961S, E961T, I962A, I962C, I962K, I962N, L965C, L965G, L965K, L965M, L965P, L965V, L965Y, L966A, L966G, L966H, L966N, L966P, L966T, N967D, N967M, N967P, N967S, N967T, Y968G, Y968L, Y968Q, Y968V, S969C, S969D, S969H, S969I, S969L, S969M, S969Q, S969Y, A970I, S971H, S971V, S971W, P973C, P973D, P973N, P973R, P973W, V974E, V974T, V974Y, G975Q, T976F, T976G, T976K, T976P, P977C, P977T, P977Y, A978F, A978M, A978N, A978R, V979R, L980A, L980H, L980I, L980N, L980Q, L980T, P981M, G982P, G982Q, P985E, P985L, P985W, A986I, A986L, A986N, A986W, V987A, V987F, V987K, L988E, L988G, L988S, P989A, P989M, D990W, G991F, G991H, G991Y, T992M, T994S, S995L, S995V, A996Q, A996V, N997A, N997E, N997K, N997L, N997Y, F998M, F998W, A999G, A999M, A999R, A999S, V1000C, V1000L, V1000M, V1000N, D1001G, D1001K, D1001L, D1001T, D1001V, W1002A, W1002D, W1002E, W1002H, W1002N, W1002P, W1002Q, T1003L, T1003P, T1003Y, K1004F, K1004H, K1004P, K1004R, K1004V, P1005I, P1005Y, A1006C, A1006I, A1006S, D1007L, D1007P, D1007V, V1009G, Y1010T, N1011A, N1011T, T1012Q, A1013Q, A1013V, G1014I, G1014L, T1015G, V1016C, V1016D, V1018K, T1021F, T1021G, T1021V, A1022H, A1022L, T1023D, V1024G, V1024H, V1024K, V1024N, V1024R, V1024S, V1024W, G1026L, G1026R, G1026S, G1026V, K1027C, K1027N, F1029K, F1029P, F1029V, K1030D, K1030W, V1031H, V1031K, T1034H, I1035D, R1036G, R1036L, R1036T, V1037F, Q1041P, V1042N, T1043F, I1044A, S1046M, V1048C, V1048F, V1048G, V1048M, V1048Q, G1050S, N1051E, N1051K, A1052K, L1053A, L1053W, L1055R, Q1057E, Q1057R, N1058R, N1058S, N1058V, I1059W, P1060G, P1060N, P1060T, A1061G, D1062A, D1062L, D1062M, K1063D, Q1064M, Q1064R, S1065A, S1065C, S1065E, D1066A, D1066G, D1066M, D1066V, D1066W, T1067M, L1068C, L1068P, L1068Q, A1070P, I1071R, I1071W, K1072E, K1072G, K1072Q, K1072S, D1073L, D1073P, G1074I, G1074L, G1074R, S1075C, S1075L, T1076H, T1076Q, T1076S, V1078L, T1082E, T1082F, G1083E, G1083F, G1083L, G1083S, G1084V, G1084Y, G1085S, A1086H, A1086Q, N1087R, N1087W, P1088E, P1088R, S1089C, S1089G, S1089K, S1089Q, S1089R, A1090G, W1091A, W1091E, W1091G, W1091H, W1091T, W1091V, W1091Y, T1092G, T1092Q, T1092S, T1092V, N1093A, N1093P, N1093Q, N1093T, N1093V, W1094D, W1094E, W1094P, A1095W, Y1096A, Y1096D, S1097L, S1097T, S1097W, K1098D, K1098F, A1099C, A1099D, A1099S, A1099V, G1100H, G1100M, G1100T, H1101V, N1102E, N1102H, N1102K, T1103E, A1104R, E1105L, I1106T, T1107R, T1107S, F1108D, F1108L, F1108T, E1109A, E1109D, E1109L, E1109W, A1111G, E1113D, E1113P, E1113V, Q1114E, Q1114R, Q1114S, Q1114T, Q1114V, Q1115A, Q1115K, Q1115L, Q1115P, L1116G, L1116K, L1116W, G1117E, G1117I, G1117M, G1117R, G1117S, G1117T, G1117V, G1117W, Q1118W, I1119E, I1119G, I1119N, I1119S, V1120T, M1121G, M1121K, M1121V, Y1122K, Y1122R, Y1122V, F1123R, F1123T, F1124V, F1124W, R1125D, R1125E, R1125K, R1125V, R1125W, D1126H, D1126L, S1127F, S1127I, S1127K, S1127Q, S1127W, N1128A, N1128R, N1128S, N1128W, A1129E, A1129L, A1129Q, A1129V, V1130A, V1130G, V1130R, V1130S, R1131A, R1131S, F1132E, P1133D, P1133G, P1133R, D1134G, D1134L, A1135L, A1135S, A1135W, A1135Y, G1136A, G1136E, G1136P, K1137C, K1137L, K1137P, K1137Q, K1137R, K1137S, T1138R, I1140C, I1140G, I1140L, I1140P, Q1141K, Q1141P, Q1141W, I1142Y, S1143G, A1144C, A1144N, A1144P, A1144S, A1144T, A1144V, A1144W, D1145E, D1145R, D1145T, G1146D, G1146L, G1146V, K1147T, K1147V, N1148P, W1149C, W1149G, W1149I, W1149N, W1149Q, W1149S, W1149V, T1150K, D1151R, D1151W, L1152A, L1152E, L1152Q, L1152W, A1153G, A1154C, A1154R, T1155L, I1158R, I1158W, A1159I, A1159R, A1159V, A1160Q, Q1161A, Q1161P, Q1161S, E1162A, E1162C, E11621, E1162Q, E1162Y, E1165L, E1165S, V1167R, K1168Q, K1168W, P1169S, Y1170E, Y1170K, T1171A, T1171G, Y1172E, Y1172K, D1173A, D1173F, D1173K, D1173L, F1174P, F1174T, F1174V, F1174W, A1175N, A1175Q, A1175S, A1175V, A1175Y, V1177N, V1177T, G1178M, G1178Q, G1178T, A1179L, A1179W, T1180A, T1180G, T1180I, T1180M, T1180Q, T1180Y, V1182M, K1183E, V1184C, V1184E, V1184K, V1184L, V1184P, V1184Q, V1184Y, V1186S, N1188W, A1189C, A1189K, A1189T, A1189V, D1190R, D1190T, T1191E, T1191L, T1192H, V1197A, V1198E, C1199T, A1200G, A1200W, L1202A, T1203K, E1204G, I1205V, K1208R, K1208V, K1208W, T1209A, T1209C, T1209E, T1209K, T1209N, T1209Q, A1210D, A1210E, A1210G, A1210K, A1210L, A1210R, A1210T, A1210W, T1211D, T1211G, T1211K, T1211P, T1211R, T1211S, K1213T, F1214A, F1214E, F1214K, F1214L, F1214P, F1214S, F1214V, V1215D, V1215L, V1215Q, T1216L, T1216P, T1216Q, N1217A, N1217F, N1217P, N1217R, N1217S, N1217T, T1218A, T1218C, T1218E, T1218G, T1218S, T1218W, S1219A, S1219F, S1219I, S1219R, A1220L, A1220P, A1220R, A1221D, A1221R, A1221V, A1221W, L1222A, L1222E, L1222F, L1222V, L1222W, S1223F, S1223K, S1223L, S1223V, S1224A, S1224D, S1224G, S1224P, S1224W, L1225C, L1225E, L1225F, L1225K, L1225W, T1226A, T1226G, T1226P, T1226S, T1226V, V1227A, V1227D, V1227L, V1227P, V1227S, N1228D, N1228F, N1228T, G1229V, T1230H, T1230I, T1230K, T1230S, T1230W, K1231F, K1231L, K1231M, K1231P, V1232K, V1232R, S1233P, S1233W, D1234K, D1234R, S1235E, S1235G, S1235R, S1235W, V1236A, V1236G, V1236I, V1236P, V1236Q, L1237D, L1237E, A1238E, A1238L, A1238N, A1238P, A1238R, A1239D, A1239P, A1239R, G1240D, G1240N, G1240W, S1241D, Y1242E, Y1242R, N1243M, N1243P, N1243T, N1243V, N1243W, T1244A, T1244E, T1244Q, T1244W, A1246M, A1246N, A1246P, A1246Q, A1246R, I1247G, I1247Q, I1247R, I1247S, I1247W, I1248G, I1248L, I1248R, I1248S, A1249G, A1249H, A1249I, A1249R, A1249T, A1249V, D1250I, D1250K, D1250S, D1250T, D1250W, D1250Y, V1251T, V1251W, K1252D, K1252V, K1252W, E1254V, G1255H, G1255M, E1256M, E1256N, E1256V, E1256W, G1257F, G1257L, G1257Q, N1258H, N1258S, A1259K, V1261I, V1261L, T1262A, T1262Q, T1262R, V1263E, V1263R, V1263T, V1263W, L1264H, P1265L, P1265R, P1265W, A1266P, A1266V, H1267E, N1269A, N1269K, N1269R, N1269S, V1270D, I1271H, R1272M, R1272V, V1273R, I1274F, I1274R, T1275L, T1275W, S1277L, S1277T, S1277W, E1278Q, D1279G, D1279I, D1279R, D1279T, D1279W, H1280E, H1280G, V1281I, V1281W, T1282D, R1283A, R1283D, R1283E, R1283W, K1284G, T1285F, F1286A, F1286E, F1286P, F1286S, T1287C, T1287R, T1287S, T1287W, I1288A, I1288D, I1288G, I1288K, N1289A, N1289T, L1290A, L1290R, G1291P, G1291V, G1291Y, T1292Y, E1293K, E1293L, E1293S, E1293V, Q1294E, Q1294P, Q1294W, E1295K, F1296A, P1297F, E1302R, E1302S, R1303S, D1304A or D1304V.

85. The lactase variant of the preceding embodiment, wherein the variant has a decreased or an increased galactose inhibition, preferably measured as a galactose inhibition improvement factor (GI-IF) of at least 1.2 or at most 0.8, compared to the lactase of SEQ ID NO: 1.

86. The lactase variant of embodiment 1 which comprises one or more of the following substitutions, wherein each position corresponds to a position in SEQ ID NO: 1: V1D, V1F, V1H, V1L, E2D, E2Q, E2V, D3I, D3V, T5S, S7N, S7P, S9H, T10L, T10S, M13C, M13D, M13K, M13R, S14V, S15C, S15I, S15K, S15P, S15R, S15T, S15V, T16C, P17I, V19K, V20C, V20G, V20L, V20M, V20P, V20Q, V20R, Y21C, Y21P, Y21R, Y21T, S22N, S22T, S22W, A24F, A24L, A24R, A24T, A24W, V25D, V25M, V25Q, V25S, D26C, D26I, D26L, S27A, S27C, S27G, S27H, S27Y, K28C, K28L, K28V, Q29G, Q29L, Q29M, Q29R, N30M, N30V, R31E, R31M, R31V, T32S, D34G, D34H, F35C, D36Q, N38S, W39G, W39S, K40C, K40F, K40G, K40I, K40W, F41A, F41C, M42T, L43A, L43I, L43S, D45P, V47K, V47R, A49D, A49H, A49T, D51G, D51I, D51K, D55C, D55F, D55G, D55H, D55M, D55P, D55S, S57A, S57C, S57E, S57G, A58Q, A58T, W59I, W59K, W59P, W59V, Q60M, Q60R, Q60S, Q60Y, Q61P, V62N, V62T, V62W, D63G, L64E, L64G, H66R, I70K, K73D, Y74K, S75G, S75L, S75Q, S75R, S75V, Q76G, Q76I, S77C, S77D, S77E, S77G, S77H, S77K, S77L, S77M, S77R, S77T, S77V, N78C, N78S, E79H, E79S, E79T, A83T, L85A, L85F, L85N, L85S, L85V, L85W, P86G, P86Q, P86R, P86V, P86W, P86Y, G87D, G87N, G88F, T89M, T89Y, G90A, G90L, G90S, G90T, W91E, W91L, W91P, W91Q, W91Y, Y92F, Y92H, Y92I, Y92M, Y92N, Y92S, Y92T, Y92V, R93A, R93D, R93F, R93H, R93I, R93L, R93N, R93T, R93V, R93Y, K94A, K94C, K94G, K94S, K94T, K94V, S95A, S95C, S95D, S95G, S95I, S95Q, S95R, F96A, F96C, F96I, F96K, F96M, F96P, F96V, F96W, T97F, T97V, I98C, I98H, I98W, R100T, D101A, D101P, D101V, L102P, R106V, R106W, I107F, I107S, A108E, I109M, N110A, N110S, N110T, N110V, N110W, F111A, F111L, F111Q, V114F, V114R, Y115E, M116C, A118P, V120A, V120K, W121D, W121R, W121V, G124M, H130A, H130C, H130S, H130T, Y132E, F137C, F137L, S138A, D140V, L141G, T142S, G149E, G149M, G149Y, E150C, E150G, E150L, E150R, V154I, V154K, V154L, V157P, V157S, E158H, E158V, N159H, N159T, L161K, L161M, L161S, L161W, P162N, P162T, P162W, S164A, R165H, W166S, Y167A, Y167C, S170Q, G171C, I172K, I172P, Y173H, Y173M, R174E, R174K, V176E, V176K, V176T, T177K, L178I, L178Q, L178W, T179A, T179C, T179D, T179I, T179K, T179N, T179P, T179S, V180A, V180D, V180G, T181A, T181F, G183W, V184F, V184P, V184R, V184S, V184W, H185G, H185L, V186E, V186N, G187D, G187H, N188R, N188V, G190F, I193N, I193Q, K194I, T195W, P196A, P196I, S197C, S197E, S197L, A199E, A199P, Q201E, N202Q, N202S, N202W, A215D, D217F, D217L, D217M, T218G, A220G, A222R, N223S, L226Q, Q228N, Q228R, F231L, G234V, G235K, K236M, K236P, T237D, G242K, G242L, G242M, G242P, T243I, A247K, I251W, I251Y, A252W, A252Y, A257D, A257N, A257V, S261H, A266D, S267V, P268F, P268R, L270N, S272T, S272W, N277F, V281Q, R282W, L286W, V291P, L292D, L292V, D293C, D293I, D293W, T294Q, T294S, Y295F, D296F, D296H, D296K, T297I, E298I, E298L, E298R, F301C, R302I, W303A, W303C, W303D, W303F, T304D, T304E, T304I, T304K, T304P, G305L, G305N, G305P, G305T, D307A, D307E, D307S, D307W, T309C, T309D, T309E, T309I, T309V, S310A, S310H, S310L, S310M, S310N, S310V, F312H, L314A, L314V, N315S, G316R, E317W, K318C, K318N, K320E, K320I, K320V, K320W, L321M, K322I, V324T, S325G, M326T, H328C, H328F, H328G, H328L, H328M, H328R, H328T, G331V, A335C, A335G, A335L, A337E, A337G, A337L, N338P, R340A, R340L, A341M, I342G, I342P, E343N, E343T, E343Y, R344G, Q345F, Q345N, Q345S, V346A, V346I, V346S, E347A, E347I, I348D, I348M, I348Q, I348R, L349A, L349S, Q350G, Q350N, M352T, G353K, V354E, V354S, V354W, I357T, T359E, T359L, T360V, N362S, N362T, P363A, P363D, P363I, P363L, P363M, P363S, P363V, A364C, A364E, A364F, A364G, A364I, A364M, A364P, A364V, A365C, A365I, A365P, A365V, A365W, K366D, K366L, K366M, K366P, K366S, K366V, A367Q, L368A, L368Q, L368S, L368Y, I369A, I369D, I369E, I369G, I369K, I369M, I369V, I369W, D370L, D370Q, V371F, V371L, V371Q, C372P, N373G, N373L, K375D, K375S, G376A, G376S, V377A, V377M, V377T, V379A, V379N, V380P, E381A, E381G, E381Q, E381T, V383A, M386G, M386N, M386S, M386V, W387H, N388E, N388L, R389A, R389C, R389E, R389Q, R389S, R389T, S390C, S390D, S390P, S390T, G393E, G393R, G393V, T395C, T395F, E396K, E396L, E396V, E396W, Y398M, G399S, K400A, K400C, K400N, K400T, K400V, W401F, W401K, F402W, F402Y, G403A, G403D, G403K, G403Q, G403V, G403Y, Q404F, Q404L, Q404M, Q404P, Q404S, Q404V, A405C, A405E, A405K, A405R, A405T, I406C, I406D, I406N, A407C, A407G, A407Q, N410R, N410Y, A411E, A411R, A411S, G414M, G414N, G415Q, K417F, K417R, A422P, L426C, T427D, T427F, T427Q, T427S, T427W, T429D, I430L, I430M, N431M, R432Q, R432Y, D433G, D433I, D433P, D433Q, D433W, R434N, R434V, N435E, N435F, N435L, N435V, A436G, A436L, V439C, V439K, W442E, W442G, S443C, S443G, S443M, S443Q, L444E, L444F, L444V, N446D, M448C, M448L, M449D, M449E, M449T, M449W, M449Y, E450F, E450S, E450T, E450V, E450W, G451C, G451F, G451L, G451P, G451V, G451W, I452K, I452L, I452Q, I452S, I452V, S453H, S453L, S453N, S453Q, G454L, G454W, S455E, S455M, S455P, S455R, V456D, V456E, V456F, V456K, S457H, S457Q, S457T, S457V, G458A, G458P, G458S, F459C, F459R, P460Y, A461G, A461M, T462E, T462L, A464W, K465L, K465V, K465Y, L466G, L466P, V467T, A468D, K471F, K471Q, A472G, A472Y, D474W, S475E, R477L, P478V, M479G, K485E, K485R, K487A, K487F, K487S, K487W, A488C, S493E, S493G, S493H, S493L, S493Q, N494M, N494R, N494V, G497D, N499T, G505D, G505H, G505L, G505R, G505S, V506C, V506E, V506R, V507R, G508C, G508E, T509A, T509I, T509K, T509M, T509V, T509Y, N510A, N510F, N510I, Y511A, S512C, S512F, S512I, S512M, S512Q, S512T, S512V, S512Y, D513P, D513R, A515E, A515G, A515P, N516C, N516E, N516G, N516I, N516Q, N516T, N516V, Y517G, D518Y, K519C, K519E, K519G, K519I, K519Q, I520A, I520C, I520G, I520H, I520M, I520N, I520Q, I520S, I520T, I520W, R521V, H524R, H524V, S526G, W527A, W527E, W527G, W527H, W527N, W527R, W527S, A528E, I529G, A535I, A537M, A537P, A537S, I538M, S540E, G549D, G549P, G549W, S553P, K556R, L558E, L558H, L558P, T559Q, T559Y, S564A, S564C, S564F, S564W, A565C, A565D, A565E, A565F, A565G, A565I, A565L, A565R, A565S, A565T, G567Q, G567V, A570G, A572W, A575D, V580D, Q581Y, R582A, R582G, R582L, D583W, F584W, A586D, Y589I, Y589Q, W591F, T592L, L597D, G598N, P600A, P600E, P600G, P600S, N604E, G607Q, G607S, S608T, G609A, A610T, W614A, N619I, N619L, V625E, V625M, V625Y, T627K, T627Q, A628D, A628N, G629T, F630C, F630Y, P631D, P631Y, K632D, K632G, T634V, F637G, F637I, F637S, F637T, F637V, Q639R, D644Y, D645S, V646R, H647G, T648C, I651T, L652C, L652W, P653Q, A654R, W655R, E657R, A661E, A661H, A661K, A661M, A661Q, A661W, K662S, K662V, K662Y, N667L, N667R, P669L, P669T, P669W, Y673S, T674M, D675A, A676P, V679Q, V679S, K680I, K680N, G687P, S688K, S688L, T689G, T689W, E690D, K691P, K691R, R692G, R692H, R692I, R692S, R692T, I694L, I694W, G695C, G695L, G695R, G695W, E696R, K697A, K697G, K697R, S698E, S698M, K701A, K701D, K701G, K701L, K701M, K701S, A705R, A705W, A706C, A706T, Y708F, Y710T, Y710V, Q711A, V712M, V712P, V712Q, Y713G, E714H, E714K, G715K, A716R, A716T, D717G, D717S, K718A, K718Q, K718T, K718Y, D719E, D719T, D719V, S720A, T721H, T721L, T721N, T721S, T721V, T721W, A722T, A722V, H723G, H723P, M726D, Y727L, L728S, T729M, N731E, N731Q, V732R, P733W, W734G, A735Q, A735R, A735S, G737F, T738S, I739K, S740D, S740F, A741I, A741P, A741S, Y744R, D745C, D745N, N747F, L750S, I751C, P752C, P752Y, G754H, G754R, T756N, E757A, N759S, N759V, G766M, A769F, A769G, A769I, A769N, A769V, A769Y, K770H, L771A, K772A, K772C, K772P, K772W, A773C, A773M, A773R, D774A, D776V, R777H, R777S, K778F, K778G, K778N, T781A, T781F, T781G, T781P, T781Y, A782Q, A782Y, D783A, K785C, K785S, K785W, K785Y, D786V, Y789V, I790R, E791S, V792S, N798I, G799K, G799L, H800V, V802I, V802S, V802Y, P803S, P803Y, A805F, A806I, A806Q, R808F, R808G, R808I, V809L, V809M, T810P, T810Q, T810Y, F811L, V813W, K814G, K814P, G815M, G815P, A816D, A816W, K818D, K818F, K818L, K818Q, K818W, K818Y, L819F, V820C, V820R, G821I, V822A, V822D, V822E, G825A, S826G, D829S, D831P, S832R, Y833E, Q834M, A835D, A835H, A835K, D836C, D836E, D836Q, D836S, D836T, D836V, D836Y, R838N, K839A, K839D, A840G, A840I, A840P, A840V, K844A, K853P, E854C, E854I, I858D, I858K, I858P, T861F, T861I, T861W, D865A, D865G, G866H, G866R, L867W, S870E, S870M, S870T, V872C, V872G, K873G, K873Y, I874G, T882A, T882R, S883L, E885V, K886L, K886V, K886W, T887A, T887D, T887G, T887N, V888A, R889G, Y892P, Y893E, S894D, Y897V, K900E, T901Q, T901R, T901Y, G902A, G902F, G902P, G902Q, G902R, G902S, G902W, N903D, N903I, K904D, K904E, K904M, K904N, K904R, K904S, K904V, P905C, P905R, P905W, P905Y, I906N, I906W, L907F, L907S, P908D, P908G, P908I, P908L, D910W, E912T, R914A, R914F, R914I, R914K, R914V, R914W, R914Y, Y915A, Y915C, Y915G, Y915I, Y915M, Y915Q, Y915V, S916G, D917C, D917E, D917F, D917L, D917M, D917Q, D917S, G918E, G918H, G918T, T919D, T919Q, T919W, D921V, R922A, R922M, R922W, Q923A, Q923V, V925C, V925K, T926R, W927P, D928A, D928Q, A929P, V930I, S931R, D933R, Q934S, Q934V, I935A, I935C, I935E, I935P, I935W, A936I, A936Q, A936Y, K937G, K937I, K937M, K937P, K937Q, A938C, A938T, G939D, S940C, S940E, S940R, S940V, F941C, S942K, S942V, V943A, T946A, T946P, T946W, V947G, V947L, V947P, V947R, V947T, A948I, A948W, G949A, K951D, K951S, K951W, K951Y, S953F, S953M, S953R, V954D, V954Q, R955C, R955W, V956D, V956Q, T957S, I959V, I959Y, D960S, E961D, E961F, E961S, E961T, I962A, I962C, I962K, I962N, L965C, L965G, L965K, L965M, L965P, L965V, L965Y, L966A, L966N, N967D, N967M, N967P, N967T, Y968G, Y968L, Y968Q, Y968V, S969C, S969D, S969H, S969I, S969L, S969M, S969Q, S969Y, A970I, S971V, S971W, P973C, P973D, P973N, P973R, P973W, V974E, V974T, V974Y, T976F, T976G, T976K, T976P, P977C, P977T, P977Y, A978F, A978M, A978N, A978R, L980A, L980H, L980I, L980N, L980Q, L980T, P981M, P985L, P985W, A986I, A986L, A986W, V987A, V987F, V987K, P989M, D990W, G991F, G991H, G991Y, T992M, S995L, N997K, F998M, F998W, D1001L, W1002A, W1002D, W1002E, W1002H, W1002N, W1002P, W1002Q, T1003Y, P1005I, P1005Y, A1006C, A1006S, D1007L, D1007P, D1007V, Y1010T, N1011A, N1011T, T1012Q, A1013Q, A1013V, G1014I, V1016D, V1018K, T1021G, A1022H, V1024G, V1024H, V1024K, V1024R, V1024S, V1024W, G1026S, G1026V, F1029K, F1029P, F1029V, K1030D, K1030W, V1031H, V1031K, T1034H, I1035D, R1036G, R1036L, R1036T, Q1041P, V1042N, T1043F, I1044A, S1046M, V1048C, V1048F, V1048G, V1048M, V1048Q, N1051E, N1051K, A1052K, L1053A, L1053W, L1055R, Q1057E, N1058R, N1058S, P1060G, P1060N, A1061G, D1062A, D1062M, Q1064M, Q1064R, S1065A, S1065C, D1066A, D1066G, D1066V, T1067M, L1068P, L1068Q, K1072S, G1074L, G1074R, S1075L, T1076S, T1082E, T1082F, G1083E, G1083F, G1083L, G1084V, G1084Y, G1085S, A1086H, N1087R, P1088E, P1088R, S1089G, S1089K, S1089Q, W1091Y, N1093A, N1093P, N1093Q, W1094D, W1094E, W1094P, A1095W, Y1096D, S1097T, K1098D, K1098F, A1099D, A1099S, A1099V, G1100H, H1101V, N1102H, T1103E, E1105L, E1109A, E1109D, E1109L, E1109W, A1111G, E1113P, E1113V, Q1114E, Q1114R, Q1114S, Q1114T, Q1114V, Q1115A, Q1115K, Q1115L, Q1115P, L1116G, L1116K, L1116W, G1117E, G1117I, G1117M, G1117R, G1117S, G1117T, G1117V, G1117W, Q1118W, I1119E, I1119N, I1119S, M1121G, M1121K, F1123R, F1124V, F1124W, R1125D, R1125K, R1125V, R1125W, S1127I, S1127Q, N1128A, A1129E, V1130A, V1130R, P1133D, P1133G, P1133R, A1135L, A1135W, G1136P, K1137P, K1137R, K1137S, T1138R, I1140C, I1140P, Q1141K, I1142Y, S1143G, A1144N, A1144P, A1144S, A1144T, A1144V, A1144W, D1145E, D1145R, D1145T, G1146V, N1148P, W1149C, W1149G, W1149S, W1149V, T1150K, D1151W, L1152A, L1152E, L1152Q, L1152W, A1153G, A1154R, I1158W, A1159I, Q1161P, Q1161S, E1162A, E1162C, E1162Q, E1165S, V1167R, P1169S, Y1170K, A1175S, G1178M, G1178Q, G1178T, A1179L, A1179W, T1180A, T1180G, T1180I, T1180M, T1180Q, T1180Y, V1182M, K1183E, V1184C, V1184E, V1184K, V1184L, V1184P, V1184Q, V1184Y, V1186S, N1188W, A1189C, A1189K, A1189T, A1189V, D1190R, D1190T, T1191L, V1197A, A1200G, T1203K, E1204G, I1205V, K1208R, K1208V, K1208W, T1209A, T1209C, T1209E, T1209K, T1209N, T1209Q, A1210D, A1210G, A1210K, A1210L, A1210R, A1210T, A1210W, T1211D, T1211G, T1211K, T1211P, T1211R, T1211S, K1213T, F1214A, F1214E, F1214K, F1214L, F1214P, F1214S, F1214V, V1215D, V1215L, V1215Q, T1216A, T1216L, T1216P, T1216Q, N1217A, N1217P, N1217R, N1217S, N1217T, T1218A, T1218C, T1218E, T1218G, T1218S, T1218W, S1219I, S1219R, A1220P, A1221R, A1221V, A1221W, L1222A, L1222E, L1222F, L1222V, L1222W, S1224P, S1224W, L1225E, L1225K, T1226P, V1227D, V1227P, G1229V, T1230H, T1230K, T1230W, V1232R, S1235E, S1235G, S1235R, S1235W, V1236A, V1236G, V1236P, V1236Q, A1238E, A1238L, A1238R, A1239R, G1240D, I1247R, G1255H, A1259K, T1262R, V1263T, L1264H, P1265W, V1273R, I1274F, I1274R, T1275W, S1277L, S1277T, S1277W, E1278Q, D1279I, D1279R, D1279W, H1280E, H1280G, V1281I, V1281W, K1284G, F1286A, F1286E, F1286P, T1287C, T1287S, T1287W, I1288A, I1288D, I1288G, I1288K, E1293K, E1293L, Q1294E, E1295K, F1296A, E1302R, E1302S, R1303S or D1304V.

87. The lactase variant of the preceding embodiment, wherein the variant has a decreased galactose inhibition, preferably measured as a galactose inhibition improvement factor (GI-IF) of at least 1.2, compared to the lactase of SEQ ID NO: 1.

88. The lactase variant of embodiment 1 which comprises one or more of the following substitutions, wherein each position corresponds to a position in SEQ ID NO: 1: V1D, V1F, V1H, V1L, E2D, E2Q, E2V, D3I, D3V, T5S, S7N, S7P, S9H, T10L, T10S, M13C, M13D, M13K, M13R, S14V, S15C, S15I, S15K, S15P, S15R, S15T, S15V, T16C, P17I, V19K, V20C, V20G, V20L, V20M, V20P, V20Q, V20R, Y21C, Y21P, Y21R, Y21T, S22N, S22T, S22W, A24F, A24L, A24R, A24T, A24W, V25D, V25M, V25Q, V25S, D26C, D26I, D26L, S27A, S27C, S27G, S27H, S27Y, K28C, K28L, K28V, Q29G, Q29L, Q29M, Q29R, N30M, N30V, R31E, R31M, R31V, T32S, D34G, D34H, F35C, D36Q, N38S, W39G, W39S, K40C, K40F, K40G, K40I, K40W, F41A, F41C, M42T, L43A, L43I, L43S, D45P, V47K, V47R, A49D, A49H, A49T, D51G, D51I, D51K, D55C, D55F, D55G, D55H, D55M, D55P, D55S, S57A, S57C, S57E, S57G, A58Q, A58T, W59I, W59K, W59P, W59V, Q60M, Q60R, Q60S, Q60Y, Q61P, V62N, V62T, V62W, D63G, L64E, L64G, H66R, I70K, K73D, Y74K, S75G, S75L, S75Q, S75R, S75V, Q76G, Q76I, S77C, S77D, S77E, S77G, S77H, S77K, S77L, S77M, S77R, S77T, S77V, N78C, N78S, E79H, E79S, E79T, A83T, L85A, L85F, L85N, L85S, L85V, L85W, P86G, P86Q, P86R, P86V, P86W, P86Y, G87D, G87N, G88F, T89M, T89Y, G90A, G90L, G90S, G90T, W91E, W91L, W91P, W91Q, W91Y, Y92F, Y92H, Y92I, Y92M, Y92N, Y92S, Y92T, Y92V, R93A, R93D, R93F, R93H, R93I, R93L, R93N, R93T, R93V, R93Y, K94A, K94C, K94G, K94S, K94T, K94V, S95A, S95C, S95D, S95G, S95I, S95Q, S95R, F96A, F96C, F96I, F96K, F96M, F96P, F96V, F96W, T97F, T97V, I98C, I98H, I98W, R100T, D101A, D101P, D101V, L102P, R106V, R106W, I107F, I107S, A108E, I109M, N110A, N110S, N110T, N110V, N110W, F111A, F111L, F111Q, V114F, V114R, Y115E, M116C, A118P, V120A, V120K, W121D, W121R, W121V, G124M, H130A, H130C, H130S, H130T, Y132E, F137C, F137L, S138A, D140V, L141G, T142S, G149M, G149Y, E150C, E150G, E150L, E150R, V154I, V154K, V154L, V157P, V157S, E158H, E158V, N159H, N159T, L161K, L161M, L161S, L161W, P162N, P162T, P162W, S164A, R165H, W166S, Y167A, Y167C, S170Q, G171C, I172K, I172P, Y173H, Y173M, R174E, R174K, V176E, V176K, V176T, T177K, L178I, L178Q, L178W, T179A, T179C, T179D, T179I, T179K, T179N, T179P, T179S, V180A, V180D, V180G, T181A, T181F, G183W, V184F, V184P, V184R, V184S, V184W, H185G, H185L, V186E, V186N, G187D, G187H, N188R, N188V, G190F, I193N, I193Q, K194I, T195W, P196A, P196I, S197C, S197E, S197L, A199E, A199P, Q201E, N202Q, N202S, N202W, A215D, D217F, D217L, D217M, T218G, A220G, A222R, N223S, L226Q, Q228N, Q228R, F231L, G234V, G235K, K236M, K236P, T237D, G242K, G242L, G242M, G242P, T243I, A247K, I251W, I251Y, A252W, A252Y, A257D, A257N, A257V, S261H, A266D, S267V, P268F, P268R, L270N, S272T, S272W, N277F, V281Q, R282W, L286W, V291P, L292D, L292V, D293C, D293I, D293W, T294Q, T294S, Y295F, D296F, D296H, D296K, T297I, E298I, E298L, E298R, F301C, R302I, W303A, W303C, W303D, W303F, T304D, T304E, T304I, T304K, T304P, G305L, G305N, G305P, G305T, D307A, D307E, D307S, D307W, T309C, T309D, T309E, T309I, T309V, S310A, S310H, S310L, S310M, S310N, S310V, F312H, L314A, L314V, N315S, G316R, E317W, K318C, K318N, K320E, K320I, K320V, K320W, L321M, K322I, V324T, S325G, M326T, H328C, H328F, H328G, H328L, H328M, H328R, H328T, G331V, A335C, A335G, A335L, A337E, A337G, A337L, N338P, R340A, R340L, A341M, I342G, I342P, E343N, E343T, E343Y, R344G, Q345F, Q345N, Q345S, V346A, V346I, V346S, E347A, E347I, I348D, I348M, I348Q, I348R, L349A, L349S, Q350G, Q350N, M352T, G353K, V354E, V354S, V354W, I357T, T359E, T359L, T360V, N362S, N362T, P363A, P363D, P363I, P363L, P363M, P363S, P363V, A364C, A364E, A364F, A364G, A364I, A364M, A364P, A364V, A365C, A365I, A365P, A365V, A365W, K366D, K366L, K366M, K366P, K366S, K366V, A367Q, L368A, L368Q, L368S, L368Y, I369A, I369D, I369E, I369G, I369K, I369M, I369V, I369W, D370L, D370Q, V371F, V371L, V371Q, C372P, N373G, N373L, K375D, K375S, G376A, G376S, V377A, V377M, V377T, V379A, V379N, V380P, E381A, E381G, E381Q, E381T, V383A, M386G, M386N, M386S, M386V, W387H, N388E, N388L, R389A, R389C, R389E, R389Q, R389S, R389T, S390C, S390D, S390P, S390T, G393E, G393R, G393V, T395C, T395F, E396K, E396L, E396V, E396W, Y398M, G399S, K400A, K400C, K400N, K400T, K400V, W401F, W401K, F402W, F402Y, G403A, G403D, G403K, G403Q, G403V, G403Y, Q404F, Q404L, Q404M, Q404P, Q404S, Q404V, A405C, A405E, A405K, A405R, I406C, I406D, I406N, A407C, A407G, A407Q, N410R, N410Y, A411E, A411R, A411S, G414M, G414N, G415Q, K417F, K417R, A422P, L426C, T427D, T427F, T427Q, T427S, T427W, T429D, I430L, I430M, N431M, R432Q, R432Y, D433G, D433I, D433P, D433Q, D433W, R434N, R434V, N435E, N435F, N435L, N435V, A436G, A436L, V439C, V439K, W442E, W442G, S443C, S443G, S443M, S443Q, L444E, L444F, L444V, N446D, M448C, M448L, M449D, M449E, M449T, M449W, M449Y, E450F, E450S, E450T, E450V, E450W, G451C, G451F, G451L, G451P, G451V, G451W, I452K, I452L, I452Q, I452S, I452V, S453H, S453L, S453N, S453Q, G454L, G454W, S455E, S455M, S455P, S455R, V456D, V456E, V456F, V456K, S457H, S457Q, S457T, S457V, G458A, G458P, G458S, F459C, F459R, P460Y, A461G, A461M, T462E, T462L, A464W, K465L, K465V, K465Y, L466G, L466P, V467T, A468D, K471F, K471Q, A472G, A472Y, D474W, S475E, R477L, P478V, M479G, K485E, K485R, K487A, K487F, K487S, K487W, A488C, S493E, S493G, S493H, S493L, S493Q, N494M, N494R, N494V, G497D, N499T, G505D, G505H, G505L, G505R, G505S, V506C, V506E, V506R, V507R, G508C, G508E, T509A, T509I, T509K, T509M, T509V, T509Y, N510A, N510F, N510I, Y511A, S512C, S512F, S512I, S512M, S512Q, S512T, S512V, S512Y, D513P, D513R, A515E, A515G, N516C, N516E, N516G, N516I, N516Q, N516T, N516V, Y517G, D518Y, K519C, K519E, K519G, K519I, K519Q, I520A, I520C, I520G, I520H, I520M, I520N, I520Q, I520S, I520T, I520W, R521V, H524R, H524V, S526G, W527A, W527E, W527G, W527H, W527N, W527R, W527S, A528E, I529G, A535I, A537M, A537P, A537S, I538M, S540E, G549D, G549P, G549W, S553P, K556R, L558E, L558H, L558P, T559Q, T559Y, S564A, S564C, S564F, S564W, A565C, A565D, A565E, A565F, A565G, A565I, A565L, A565R, A565S, A565T, G567Q, G567V, A570G, A572W, A575D, V580D, Q581Y, R582A, R582G, R582L, D583W, F584W, A586D, Y589I, Y589Q, W591F, T592L, L597D, G598N, P600A, P600E, P600G, P600S, N604E, G607Q, G607S, S608T, G609A, A610T, W614A, N619I, N619L, V625E, V625M, V625Y, T627K, T627Q, A628D, A628N, G629T, F630C, F630Y, P631D, P631Y, K632D, K632G, T634V, F637G, F637I, F637S, F637T, F637V, Q639R, D644Y, D645S, V646R, H647G, T648C, I651T, L652C, L652W, P653Q, A654R, W655R, E657R, A661E, A661H, A661K, A661M, A661Q, A661W, K662S, K662V, K662Y, N667L, N667R, P669L, P669T, P669W, Y673S, T674M, D675A, A676P, V679Q, V679S, K680I, K680N, G687P, S688K, S688L, T689G, T689W, E690D, K691P, K691R, R692G, R692H, R692I, R692S, R692T, I694L, I694W, G695C, G695L, G695R, G695W, E696R, K697A, K697G, K697R, S698E, S698M, K701A, K701D, K701G, K701L, K701M, K701S, A705R, A705W, A706C, A706T, Y708F, Y710T, Y710V, Q711A, V712M, V712P, V712Q, Y713G, E714H, E714K, G715K, A716R, D717G, D717S, K718A, K718Q, K718T, K718Y, D719E, D719T, D719V, S720A, T721H, T721L, T721N, T721S, T721V, T721W, A722T, H723G, H723P, M726D, Y727L, L728S, T729M, N731E, N731Q, V732R, P733W, W734G, A735Q, A735R, A735S, G737F, T738S, I739K, S740D, S740F, A741I, A741P, A741S, Y744R, D745C, D745N, N747F, L750S, I751C, P752C, P752Y, G754H, T756N, E757A, N759S, N759V, G766M, A769F, A769G, A769I, A769N, A769V, A769Y, K770H, L771A, K772A, K772C, K772P, K772W, A773C, A773M, A773R, D774A, D776V, R777H, R777S, K778F, K778G, K778N, T781A, T781F, T781G, T781P, T781Y, A782Q, A782Y, D783A, K785C, K785S, K785W, K785Y, D786V, Y789V, I790R, E791S, V792S, N798I, G799K, G799L, H800V, V802I, V802S, V802Y, P803S, P803Y, A805F, A806I, A806Q, R808F, R808G, R808I, V809L, V809M, T810P, T810Q, T810Y, F811L, V813W, K814G, K814P, G815M, G815P, A816D, A816W, K818D, K818F, K818L, K818Q, K818W, K818Y, L819F, V820C, V820R, G821I, V822A, V822D, V822E, G825A, S826G, D829S, D831P, S832R, Y833E, Q834M, A835D, A835H, A835K, D836C, D836E, D836Q, D836S, D836T, D836V, D836Y, R838N, K839A, K839D, A840G, A840I, A840P, A840V, K844A, K853P, E854C, E854I, I858D, I858K, I858P, T861F, T861I, T861W, D865A, D865G, G866H, G866R, L867W, S870E, S870M, S870T, V872C, V872G, K873G, K873Y, I874G, T882A, T882R, S883L, E885V, K886L, K886V, K886W, T887A, T887D, T887G, T887N, V888A, R889G, Y892P, Y893E, S894D, Y897V, K900E, T901Q, T901R, T901Y, G902A, G902F, G902P, G902Q, G902R, G902S, G902W, N903D, N903I, K904D, K904E, K904M, K904N, K904R, K904S, K904V, P905C, P905R, P905W, P905Y, I906W, L907F, L907S, P908D, P908G, P908I, P908L, D910W, E912T, R914A, R914F, R914I, R914K, R914V, R914W, R914Y, Y915A, Y915C, Y915G, Y915I, Y915M, Y915Q, Y915V, S916G, D917C, D917E, D917F, D917L, D917M, D917Q, D917S, G918E, G918H, G918T, T919D, T919Q, T919W, D921V, R922A, R922M, R922W, Q923A, Q923V, V925C, V925K, T926R, W927P, D928A, D928Q, A929P, V930I, S931R, D933R, Q934S, Q934V, I935A, I935C, I935E, I935P, I935W, A936I, A936Q, A936Y, K937G, K937I, K937M, K937P, K937Q, A938C, A938T, G939D, S940C, S940E, S940R, S940V, F941C, S942K, S942V, V943A, T946A, T946P, T946W, V947G, V947L, V947P, V947R, V947T, A948I, A948W, G949A, K951D, K951S, K951W, K951Y, S953F, S953M, S953R, V954D, V954Q, R955C, R955W, V956D, V956Q, T957S, I959V, I959Y, D960S, E961D, E961F, E961S, E961T, I962A, I962C, I962K, I962N, L965C, L965G, L965K, L965M, L965P, L965V, L965Y, L966A, L966N, N967D, N967M, N967P, N967T, Y968G, Y968L, Y968Q, Y968V, S969C, S969D, S969H, S969I, S969L, S969M, S969Q, S969Y, A970I, S971V, S971W, P973C, P973D, P973N, P973R, P973W, V974E, V974T, V974Y, T976F, T976G, T976K, T976P, P977C, P977T, P977Y, A978F, A978M, A978N, A978R, L980A, L980H, L980I, L980N, L980Q, L980T, P981M, P985L, P985W, A986I, A986L, A986W, V987A, V987F, V987K, P989M, D990W, G991F, G991H, G991Y, T992M, S995L, N997K, F998M, F998W, D1001L, W1002A, W1002D, W1002E, W1002H, W1002N, W1002P, W1002Q, T1003Y, P1005I, P1005Y, A1006C, A1006S, D1007L, D1007P, D1007V, Y1010T, N1011A, N1011T, T1012Q, A1013Q, A1013V, G1014I, V1016D, V1018K, T1021G, A1022H, V1024G, V1024H, V1024K, V1024R, V1024S, V1024W, G1026S, G1026V, F1029K, F1029P, F1029V, K1030D, K1030W, V1031H, V1031K, T1034H, I1035D, R1036G, R1036L, R1036T, Q1041P, V1042N, T1043F, I1044A, S1046M, V1048C, V1048F, V1048G, V1048M, V1048Q, N1051E, N1051K, A1052K, L1053A, L1053W, L1055R, Q1057E, N1058R, N1058S, P1060G, P1060N, A1061G, D1062A, D1062M, Q1064M, Q1064R, S1065A, S1065C, D1066A, D1066G, D1066V, T1067M, L1068P, L1068Q, K1072S, G1074L, G1074R, S1075L, T1076S, T1082E, T1082F, G1083E, G1083F, G1083L, G1084V, G1084Y, G1085S, A1086H, N1087R, P1088E, P1088R, S1089G, S1089K, S1089Q, W1091Y, N1093A, N1093P, N1093Q, W1094D, W1094E, W1094P, A1095W, Y1096D, S1097T, K1098D, K1098F, A1099D, A1099S, A1099V, G1100H, H1101V, N1102H, T1103E, E1105L, E1109A, E1109D, E1109L, E1109W, A1111G, E1113P, E1113V, Q1114E, Q1114R, Q1114S, Q1114T, Q1114V, Q1115A, Q1115K, Q1115L, Q1115P, L1116G, L1116K, L1116W, G1117E, G1117I, G1117M, G1117R, G1117S, G1117T, G1117V, G1117W, Q1118W, I1119E, I1119N, I1119S, M1121G, M1121K, F1123R, F1124V, F1124W, R1125D, R1125K, R1125V, R1125W, S1127I, S1127Q, N1128A, A1129E, V1130A, V1130R, P1133D, P1133G, P1133R, A1135L, A1135W, G1136P, K1137P, K1137R, K1137S, T1138R, I1140C, I1140P, Q1141K, I1142Y, S1143G, A1144N, A1144P, A1144S, A1144T, A1144V, A1144W, D1145E, D1145R, D1145T, G1146V, N1148P, W1149C, W1149G, W1149S, W1149V, T1150K, D1151W, L1152A, L1152E, L1152Q, L1152W, A1153G, A1154R, I1158W, A1159I, Q1161P, Q1161S, E1162A, E1162C, E1162Q, E1165S, V1167R, P1169S, Y1170K, A1175S, G1178M, G1178Q, G1178T, A1179L, A1179W, T1180A, T1180G, T1180I, T1180M, T1180Q, T1180Y, V1182M, K1183E, V1184C, V1184E, V1184K, V1184L, V1184P, V1184Q, V1184Y, V1186S, N1188W, A1189C, A1189K, A1189T, A1189V, D1190R, D1190T, T1191L, V1197A, A1200G, T1203K, E1204G, I1205V, K1208R, K1208V, K1208W, T1209A, T1209C, T1209E, T1209K, T1209N, T1209Q, A1210D, A1210G, A1210K, A1210L, A1210R, A1210T, A1210W, T1211D, T1211G, T1211K, T1211P, T1211R, T1211S, K1213T, F1214A, F1214E, F1214K, F1214L, F1214P, F1214S, F1214V, V1215D, V1215L, V1215Q, T1216L, T1216P, T1216Q, N1217A, N1217P, N1217R, N1217S, N1217T, T1218A, T1218C, T1218E, T1218G, T1218S, T1218W, S1219I, S1219R, A1220P, A1221R, A1221V, A1221W, L1222A, L1222E, L1222F, L1222V, L1222W, S1224P, S1224W, L1225E, L1225K, T1226P, V1227D, V1227P, G1229V, T1230H, T1230K, T1230W, V1232R, S1235E, S1235G, S1235R, S1235W, V1236A, V1236G, V1236P, V1236Q, A1238E, A1238L, A1238R, A1239R, G1240D, I1247R, G1255H, A1259K, T1262R, V1263T, L1264H, P1265W, V1273R, I1274F, I1274R, T1275W, S1277L, S1277T, S1277W, E1278Q, D1279I, D1279R, D1279W, H1280E, H1280G, V1281I, V1281W, K1284G, F1286A, F1286E, F1286P, T1287C, T1287S, T1287W, I1288A, I1288D, I1288G, I1288K, E1293K, E1293L, Q1294E, E1295K, F1296A, E1302R, E1302S, R1303S or D1304V.

89. The lactase variant of the preceding embodiment, wherein the variant has a decreased galactose inhibition, preferably measured as a galactose inhibition improvement factor (GI-IF) of at least 1.2, compared to the lactase of SEQ ID NO: 1.

90. The lactase variant of embodiment 1 which comprises one or more of the following substitutions, wherein each position corresponds to a position in SEQ ID NO: 1: V1H, V1L, E2D, E2V, D3I, D3S, D3V, D3W, R6M, R6P, S7D, S7N, S7P, M13C, M13F, M13R, S14V, S15C, S15I, S15K, S15T, S15V, S15Y, T16C, P17D, P17I, P17L, V19G, V20C, V20M, V20P, Y21C, Y21P, Y21R, Y21T, S22W, S23L, S23M, A24F, A24L, A24T, A24W, V25K, V25Q, D26C, S27A, S27C, S27H, K28C, Q29L, Q29M, Q29R, Q29V, R31E, R31V, T32S, S33C, S33Q, S33R, D34S, D34Y, F35C, F35E, F35N, W39G, K40C, K40F, K40I, F41A, F41G, M42E, M42T, L43C, L43S, L43T, L43V, D45P, V47K, V47R, A49D, A49H, A49R, A49T, D51I, D51K, D51P, D51V, A53S, D55C, D55H, D55M, D55P, S57C, S57E, S57G, A58G, A58Q, A58T, W59I, W59K, W59P, Q60K, Q60R, Q60S, V62N, V62S, V62T, V62W, L64E, H66R, H66Y, Y68P, K73D, S75G, S75L, S75R, S75V, Q76G, Q76I, S77D, S77E, S77F, S77G, S77H, S77K, S77R, N78C, N78E, E79H, E79Q, E79S, E79T, E81A, E81Q, A83T, L85A, L85F, L85N, L85S, L85V, L85W, P86Q, G87D, G88F, G88Q, G90T, W91P, W91Y, Y92H, Y92I, Y92M, Y92S, Y92T, Y92V, R93A, R93D, R93F, R93H, R931, R93L, R93N, R93T, R93V, R93Y, K94A, K94S, K94T, S95C, S95D, S95G, S95I, F96A, F96C, F96I, F96K, F96M, F96P, F96S, F96V, T97F, I98C, I98H, R100T, D101A, D101P, L102P, R106W, I107G, I107S, A108E, I109M, N110A, N110S, N110T, N110W, F111A, F111L, D112T, V114R, Y115E, M116C, A118K, A118P, A118Y, V120A, V120K, W121R, W121V, W121Y, F122S, F122Y, N123P, G124E, H130A, H130T, Y132C, Y132E, P136R, F137L, T142S, K146A, G149M, E150L, I153A, V154K, V154L, V157L, V157P, V157S, E158G, E158H, N159T, L161K, L161M, L161S, L161W, P162N, P162T, P162W, S164A, R165H, W166S, Y167A, G169D, S170Q, I172K, I172P, Y173H, R174E, V176E, V176K, T177K, L178I, L178Q, T179C, T179I, T179N, V180D, V180G, T181A, V184R, H185G, H185L, G187D, N188E, N188V, N189E, G190C, G190F, K194I, T195S, S197C, L198R, A199P, Q201E, N202Q, N202R, N202S, N202W, G203K, G203V, G204Y, N205E, N205G, N205H, V206K, T207Q, N209D, N209K, T211R, T212E, K213C, K213D, V214A, A215Q, D217L, D217M, T218G, A220G, A220V, A221E, A222R, N223S, Q228R, T229G, T229V, V230M, F231G, F231V, F231Y, P232S, K233C, K233L, K233P, G234L, G234Y, G235H, G235M, G235Y, K236D, K236L, K236M, K236W, K236Y, T237D, D238E, D238F, D238P, A239G, A239T, A240C, G242K, G242Y, T243M, T245G, T245Q, A247P, A247R, A247W, S248E, S248T, K249G, K249N, K249Y, I251Y, A252F, A252W, A252Y, G254I, G254M, G254R, G254W, A255K, A255S, A255Y, S256A, S256K, S256N, S256Y, D258L, D258W, S261H, S261R, S261W, T262D, T264M, A266D, A266K, A266Q, S267V, P268R, S272T, S272W, L278H, L278M, L278Q, L278R, L278V, Y279W, T280E, V281Q, R282H, R282I, E284L, V285T, L286F, V291D, V291P, L292H, L292V, D293C, D293S, D293W, Y295F, Y295W, D296F, D296K, D296R, E298I, E298R, F301C, W303A, W303C, W303D, T304D, T304K, T304P, G305N, G305P, G305W, A308M, A308Y, T309C, T309D, T309E, S310H, S310L, S310N, G311V, F312H, L314A, L314C, L314V, N315S, K318N, K320C, K320V, K320W, L321M, K322F, K322I, V324T, S325D, S325G, S325T, M326E, M326G, M326T, M326Y, H328C, H328F, H328G, H328M, H328T, G331V, A335G, A335L, A337G, A337L, R340A, R340L, A341M, I342G, I342K, E343N, E343Y, R344G, Q345G, Q345K, Q345S, V346A, E347A, I348D, I348M, Q350G, M352T, M352W, G353K, I357T, T359E, T359L, T360V, N362S, N362T, P363A, P363I, P363L, P363M, A364I, A364M, A364P, A365C, A365I, A365P, A365V, K366L, K366M, K366S, L368A, L368E, L368Q, L368S, I369E, I369G, I369K, I369V, I369W, D370L, V371D, V371F, V371Q, C372P, N373G, N373L, G376A, G376S, V377M, V377T, V379A, V379N, V380P, E381A, E381G, E381Q, E381T, V383A, M386G, M386N, M386S, W387H, W387L, N388L, N388R, R389A, R389E, R389Q, R389S, R389T, S390C, S390D, S390G, S390P, S390Q, S390T, S390V, K391E, N392D, G393A, G393E, G393R, G393S, G393V, N394L, T395C, T395H, T395N, T395S, T395W, E396K, E396L, E396M, E396V, E396W, Y398M, Y398N, G399S, K400A, K400C, K400N, K400P, K400Q, K400S, K400V, W401F, W401K, W401L, F402W, F402Y, G403A, G403K, G403Q, G403V, Q404F, Q404H, Q404L, Q404M, Q404P, Q404V, A405C, A405E, A405H, A405K, A405R, A405T, I406D, I406N, A407C, A407G, A407Q, A407T, G408W, D409N, N410Y, A411E, A411V, L413P, G414A, G414M, G414N, G414R, G415R, D416I, D416R, D416Y, K417F, K417R, D418P, D418R, E419M, E419R, E419W, T420K, T420R, W421Q, A422P, A422T, K423D, F424C, F424L, F424N, L426M, T427D, T427F, T427K, T427M, T427Q, T427S, T429D, I430S, N431M, D433G, D433I, D433P, D433Q, D433W, R434N, R434V, N435E, N435L, N435R, A436L, P437Q, P437R, V439C, V439E, V439G, V439K, V439T, I440R, M441A, W442E, W442G, W442R, S443C, S443G, S443Q, S443Y, L444E, L444G, L444V, G445A, G445C, G445V, N446D, N446T, M448C, M448L, M448Q, M448S, M448V, M449D, M449T, M449W, M449Y, E450F, E450S, E450T, E450V, E450W, G451C, G451F, G451L, G451P, G451V, G451W, I452G, I452K, I452M, I452S, I452V, G454L, S455P, S455R, S455W, V456D, V456E, V456F, V456K, S457H, S457T, S457V, G458A, G458P, G458S, G458W, F459N, F459R, F459S, A461M, T462E, T462L, S463V, A464P, A464V, K465F, K465V, K465Y, L466A, L466E, L466P, L466S, L466V, A468D, A468W, T470M, K471F, A472G, A472Y, D474R, S475E, R477L, M479G, M479I, M479R, T480C, T480G, K485E, K485R, K487F, K487N, K487S, K487W, A488H, A488N, A488S, N491W, E492A, S493G, S493L, S493M, S493Q, N494R, T495R, T495W, G497D, D498C, D498S, N499R, N499T, N499Y, L500A, T501M, G504H, G504P, G505D, G505N, G505R, G505S, V506C, V506D, V506E, V506G, V506R, V507L, V507R, G508C, G508E, T509A, T509K, T509M, N510A, N510F, N510I, Y511A, S512F, S512Q, S512T, S512V, S512Y, D513K, D513L, D513P, D513R, G514F, A515C, A515E, A515K, A515P, A515R, N516C, N516E, N516Q, N516V, Y517G, D518Y, K519C, K519I, I520C, I520G, I520H, I520M, I520S, I520T, R521N, R521V, T522G, T522N, H524R, H524V, P525T, S526G, W527A, W527H, W527N, W527R, W527S, A528E, Y530M, G531E, G531S, G531T, T534A, A535M, A537D, A537M, A537P, S540E, S540G, G542Q, I543V, N545S, R546L, R546P, R546S, T547A, T547D, T547S, T548E, T548F, T548K, T548P, G549D, G549P, G549W, G550S, A551I, A551Q, S553N, S554N, S554V, D555S, K556W, L558E, L558P, T559Y, Y561R, N563S, S564C, S564F, S564W, A565D, A565E, A565I, A565L, A565R, A565T, G567Q, G567V, A570G, A570K, A570M, A570W, A572S, S573K, S574K, S574Q, S574W, A575D, A575V, W576Y, Y577G, Y577L, D578N, V579G, V579T, V580K, V580S, Q581G, R582L, D583W, F584E, V585I, A586K, G587C, T588D, T588I, Y589V, Y589W, V590H, W591F, T592C, T592L, T592S, F594L, D595E, D595Q, D595S, G598N, P600A, P600E, P600G, P600S, T606S, G607Q, S608V, G609N, G609R, A610M, V611K, W614A, W614P, N619I, N619L, S620G, V625M, V625Y, T627K, T627Q, A628D, F630C, F630D, F630G, F630Y, P631A, P631D, T634V, Y635R, Q639R, D644C, H650E, H650R, W655R, N656V, E657R, A661E, A661K, A661Q, N667L, N667R, P669T, V670C, Y673R, Y673S, T674M, D675P, A676P, A677G, K678T, V679G, V679S, K680I, L681F, T684G, T684V, G687P, S688K, S688L, T689W, E690D, R692G, R692H, I694L, I694W, G695C, G695L, G695W, K697R, S698E, S698L, S698M, T700C, K701A, K701G, K701M, K701S, T704Y, A705E, A705W, Y708F, T709M, Y710D, Y710T, Y710W, Q711Y, V712M, V712Q, E714K, A716R, D717G, D717S, K718A, K718T, D719E, D719T, D719V, T721H, T721L, T721N, T721S, T721V, T721W, H723G, M726D, Y727L, L728S, N731E, N731S, V732R, W734G, W734V, A735Q, A735S, T738S, I739K, S740D, A741I, Y744R, D745F, D745N, N747R, L750S, P752A, P752C, P752Y, G754P, T756N, T756S, E757A, A760N, A769F, A769I, A769N, A769V, A769Y, K770H, L771A, K772A, K772C, K772P, K772V, A773C, A773H, A773M, A773R, A773V, D774A, D774R, A775L, A775W, D776I, D776L, D776S, D776V, R777D, R777E, R777G, R777H, R777P, R777S, K778L, K778N, T781A, T781F, T781G, T781M, T781P, T781Y, A782P, A782Q, D783A, D783C, D783E, D783R, G784L, G784S, G784T, K785S, K785W, K785Y, D786V, L787P, L787T, S788G, S788I, Y789V, I790C, I790R, V792G, D793H, V794C, V794T, V794W, T795P, D796Q, N798I, G799K, G799L, G799M, G799Q, H800A, H800L, V802Y, P803S, D804G, A806I, A806Q, N807W, R808C, R808F, R808N, T810P, T810Q, D812Q, V813T, V813W, K814H, K814L, G815A, G815V, A816C, A816F, K818F, K818L, K818W, K818Y, L819F, V820K, G821E, G821F, G821I, G821N, V822D, V822E, S826G, S826L, P828C, D829S, H830E, H830R, H830V, D831F, D831I, D831P, D831R, D831V, S832R, Y833K, Y833V, A835D, A835E, A835H, A835K, A835W, D836C, D836E, D836H, D836S, D836V, D836Y, N837D, R838G, R838M, R838N, K839A, K839D, A840G, A840P, A840V, F841C, F841W, K844G, V845W, V849L, V849S, Q850C, Q850G, Q850Y, E854C, E857P, I858D, I858Q, I858Y, T859V, V860T, T861W, A862V, K863F, K863N, K863W, D865A, D865G, G866H, L867W, S870E, S870R, V872C, K873G, K873Y, I874G, T877A, G881W, S883L, T884A, K886E, K886V, K886W, T887F, T887G, V888A, Y892P, Y892R, Y893E, N896M, Y897V, K900E, T901G, T901Q, T901R, T901V, T901Y, G902F, G902L, G902R, G902S, G902W, N903D, K904M, K904N, K904S, K904V, P905C, P905R, P905Y, I906W, L907F, P908I, P908L, D910W, E912T, R914A, R914F, R914I, R914K, R914V, R914Y, Y915A, Y915C, Y915G, Y915M, Y915Q, Y915V, S916G, D917C, D917F, D917S, G918E, G918H, G918T, T919D, T919Q, S920E, D921V, R922A, Q923V, T926G, T926R, W927G, W927P, A929P, D933R, Q934S, I935A, I935C, I935E, I935P, I935W, A936I, A936Q, K937G, K937I, K937P, K937Q, A938C, G939D, S940C, S940T, F941C, S942K, S942V, V943A, V943H, V943R, A944D, A944H, A944P, A944R, T946P, T946V, V947G, V947M, V947R, V947T, A948C, A948R, A948W, Q950D, K951S, S953M, S953W, R955C, R955W, V956D, V956Q, M958D, E961D, E961F, E961S, I962C, I962K, I962N, L965C, L965G, L965K, L965M, L965P, L965Y, L966A, L966H, N967M, Y968G, Y968Q, Y968V, S969C, S969I, S969L, S969M, S969Q, A970I, S971V, P973C, P973W, V974T, T976K, T976P, P977C, P977T, A978F, A978M, V979R, L980H, L980N, P981M, P985L, P985W, A986I, A986L, V987A, V987F, V987K, D990W, G991Y, S995L, N997K, A999G, V1000N, D1001L, W1002A, W1002D, W1002E, W1002H, W1002N, W1002P, W1002Q, T1003L, T1003Y, K1004H, P1005Y, A1006C, A1006S, D1007P, N1011T, T1012Q, G1014I, V1018K, T1021F, A1022H, V1024G, V1024H, G1026R, G1026V, K1030D, K1030W, I1035D, Q1041P, V1042N, V1048C, V1048G, V1048M, V1048Q, G1050S, N1051E, N1051K, A1052K, L1053A, Q1057E, Q1057R, N1058V, I1059W, A1061G, D1062L, D1062M, Q1064M, S1065A, D1066M, D1066V, D1066W, T1067M, L1068C, L1068P, A1070P, I1071W, K1072G, K1072Q, K1072S, D1073P, G1074I, G1074L, S1075C, T1082F, G1083E, G1083F, G1083L, G1083S, G1084V, G1085S, P1088E, P1088R, S1089C, S1089G, S1089Q, W1091A, W1091E, W1091G, W1091T, W1091V, W1091Y, T1092G, T1092Q, N1093A, N1093P, N1093Q, W1094D, W1094E, S1097W, K1098D, A1099V, G1100M, H1101V, N1102E, N1102H, T1103E, A1104R, I1106T, T1107S, F1108D, F1108L, E1109A, E1109D, E1109L, E1109W, A1111G, E1113P, E1113V, Q1114E, Q1114R, Q1114S, Q1114T, Q1114V, Q1115A, Q1115K, L1116K, L1116W, G1117I, G1117R, G1117S, G1117T, G1117V, G1117W, I1119N, M1121G, M1121V, R1125V, R1125W, D1126H, S1127I, S1127Q, N1128A, A1129E, A1129Q, A1129V, V1130A, V1130G, V1130R, P1133D, P1133R, D1134L, A1135L, A1135S, A1135W, G1136A, G1136E, G1136P, K1137C, K1137L, K1137Q, K1137R, K1137S, I1140G, I1140L, I1140P, Q1141K, Q1141P, I1142Y, A1144C, A1144N, A1144P, A1144S, A1144V, A1144W, D1145E, D1145R, D1145T, N1148P, W1149C, W1149I, W1149V, T1150K, D1151R, D1151W, L1152W, A1153G, A1154C, E1162A, E1165L, V1167R, K1168W, Y1170E, T1171A, T1171G, A1175N, A1175Q, V1177N, V1177T, G1178M, G1178T, A1179L, A1179W, T1180A, T1180G, T1180I, T1180M, T1180Q, T1180Y, V1184E, V1184L, V1184P, V1184Q, V1184Y, V1186S, N1188W, D1190T, T1191L, T1192H, V1197A, C1199T, A1200W, L1202A, E1204G, K1208W, T1209E, T1209K, A1210D, A1210E, A1210G, A1210K, A1210L, A1210R, A1210T, A1210W, T1211G, T1211K, T1211P, T1211R, T1211S, F1214A, F1214K, F1214S, F1214V, V1215D, V1215L, T1216L, T1216Q, N1217A, N1217S, N1217T, T1218C, T1218E, T1218S, T1218W, S1219A, S1219F, A1220L, L1222A, L1222E, L1222F, L1222V, S1223V, S1224A, S1224G, S1224P, S1224W, L1225E, L1225K, L1225W, T1226P, T1226S, T1226V, N1228D, N1228F, G1229V, T1230I, T1230K, T1230W, K1231F, K1231P, V1232K, V1232R, S1233P, S1233W, S1235W, V1236A, V1236G, V1236P, L1237D, A1238E, A1238R, G1240W, Y1242E, Y1242R, N1243M, N1243T, T1244Q, A1246M, I1248G, A1249H, A1249I, A1249V, D1250I, D1250K, D1250S, D1250T, D1250W, D1250Y, K1252D, G1255H, N1258H, A1259K, T1262R, V1263T, V1263W, P1265R, P1265W, N1269A, N1269K, I1274F, I1274R, T1275W, S1277L, S1277T, S1277W, E1278Q, D1279R, D1279W, H1280E, V1281I, V1281W, K1284G, F1286E, F1286P, T1287C, T1287S, T1287W, I1288A, I1288D, I1288G, I1288K, G1291P, G1291Y, T1292Y, E1295K, F1296A, E1302R, E1302S or D1304V.

91. The lactase variant of the preceding embodiment, wherein the variant has a decreased or an increased galactose inhibition, preferably measured as a galactose inhibition improvement factor (GI-IF) of at least 1.3 or at most 0.7, compared to the lactase of SEQ ID NO: 1.

92. The lactase variant of embodiment 1 which comprises one or more of the following substitutions, wherein each position corresponds to a position in SEQ ID NO: 1: V1H, V1L, E2D, E2V, D3I, D3S, D3V, D3W, R6M, R6P, S7D, S7N, S7P, M13C, M13F, M13R, S14V, S15C, S15I, S15K, S15T, S15V, S15Y, T16C, P17D, P17I, P17L, V19G, V20C, V20M, V20P, Y21C, Y21P, Y21R, Y21T, S22W, S23L, S23M, A24F, A24L, A24T, A24W, V25K, V25Q, D26C, S27A, S27C, S27H, K28C, Q29L, Q29M, Q29R, Q29V, R31E, R31V, T32S, S33C, S33Q, S33R, D34S, D34Y, F35C, F35E, F35N, W39G, K40C, K40F, K40I, F41A, F41G, M42E, M42T, L43C, L43S, L43T, L43V, D45P, V47K, V47R, A49D, A49H, A49R, A49T, D51I, D51K, D51P, D51V, A53S, D55C, D55H, D55M, D55P, S57C, S57E, S57G, A58G, A58Q, A58T, W59I, W59K, W59P, Q60K, Q60R, Q60S, V62N, V62S, V62T, V62W, L64E, H66R, H66Y, Y68P, K73D, S75G, S75L, S75R, S75V, Q76G, Q76I, S77D, S77E, S77F, S77G, S77H, S77K, S77R, N78C, N78E, E79H, E79Q, E79S, E79T, E81A, E81Q, A83T, L85A, L85F, L85N, L85S, L85V, L85W, P86Q, G87D, G88F, G88Q, G90T, W91P, W91Y, Y92H, Y92I, Y92M, Y92S, Y92T, Y92V, R93A, R93D, R93F, R93H, R931, R93L, R93N, R93T, R93V, R93Y, K94A, K94S, K94T, S95C, S95D, S95G, S95I, F96A, F96C, F96I, F96K, F96M, F96P, F96S, F96V, T97F, I98C, I98H, R100T, D101A, D101P, L102P, R106W, I107G, I107S, A108E, I109M, N110A, N110S, N110T, N110W, F111A, F111L, D112T, V114R, Y115E, M116C, A118K, A118P, A118Y, V120A, V120K, W121R, W121V, W121Y, F122S, F122Y, N123P, G124E, H130A, H130T, Y132C, Y132E, P136R, F137L, T142S, K146A, G149M, E150L, I153A, V154K, V154L, V157L, V157P, V157S, E158G, E158H, N159T, L161K, L161M, L161S, L161W, P162N, P162T, P162W, S164A, R165H, W166S, Y167A, G169D, S170Q, I172K, I172P, Y173H, R174E, V176E, V176K, T177K, L178I, L178Q, T179C, T179I, T179N, V180D, V180G, T181A, V184R, H185G, H185L, G187D, N188E, N188V, N189E, G190C, G190F, K194I, T195S, S197C, L198R, A199P, Q201E, N202Q, N202R, N202S, N202W, G203K, G203V, G204Y, N205E, N205G, N205H, V206K, T207Q, N209D, N209K, T211R, T212E, K213C, K213D, V214A, A215Q, D217L, D217M, T218G, A220G, A220V, A221E, A222R, N223S, Q228R, T229G, T229V, V230M, F231G, F231V, F231Y, P232S, K233C, K233L, K233P, G234L, G234Y, G235H, G235M, G235Y, K236D, K236L, K236M, K236W, K236Y, T237D, D238E, D238F, D238P, A239G, A239T, A240C, G242K, G242Y, T243M, T245G, T245Q, A247P, A247R, A247W, S248E, S248T, K249G, K249N, K249Y, I251Y, A252F, A252W, A252Y, G254I, G254M, G254R, G254W, A255K, A255S, A255Y, S256A, S256K, S256N, S256Y, D258L, D258W, S261H, S261R, S261W, T262D, T264M, A266D, A266K, A266Q, S267V, P268R, S272T, S272W, L278H, L278M, L278Q, L278R, L278V, Y279W, T280E, V281Q, R282H, R282I, E284L, V285T, L286F, V291D, V291P, L292H, L292V, D293C, D293S, D293W, Y295F, Y295W, D296F, D296K, D296R, E298I, E298R, F301C, W303A, W303C, W303D, T304D, T304K, T304P, G305N, G305P, G305W, A308M, A308Y, T309C, T309D, T309E, S310H, S310L, S310N, G311V, F312H, L314A, L314C, L314V, N315S, K318N, K320C, K320V, K320W, L321M, K322F, K322I, V324T, S325D, S325G, S325T, M326E, M326G, M326T, M326Y, H328C, H328F, H328G, H328M, H328T, G331V, A335G, A335L, A337G, A337L, R340A, R340L, A341M, I342G, I342K, E343N, E343Y, R344G, Q345G, Q345K, Q345S, V346A, E347A, I348D, I348M, Q350G, M352T, M352W, G353K, I357T, T359E, T359L, T360V, N362S, N362T, P363A, P363I, P363L, P363M, A364I, A364M, A364P, A365C, A365I, A365P, A365V, K366L, K366M, K366S, L368A, L368E, L368Q, L368S, I369E, I369G, I369K, I369V, I369W, D370L, V371D, V371F, V371Q, C372P, N373G, N373L, G376A, G376S, V377M, V377T, V379A, V379N, V380P, E381A, E381G, E381Q, E381T, V383A, M386G, M386N, M386S, W387H, W387L, N388L, N388R, R389A, R389E, R389Q, R389S, R389T, S390C, S390D, S390G, S390P, S390Q, S390T, S390V, K391E, N392D, G393A, G393E, G393R, G393S, G393V, N394L, T395C, T395H, T395N, T395S, T395W, E396K, E396L, E396M, E396V, E396W, Y398M, Y398N, G399S, K400A, K400C, K400N, K400P, K400Q, K400S, K400V, W401F, W401K, W401L, F402W, F402Y, G403A, G403K, G403Q, G403V, Q404F, Q404H, Q404L, Q404M, Q404P, Q404V, A405C, A405E, A405H, A405K, A405R, I406D, I406N, A407C, A407G, A407Q, A407T, G408W, D409N, N410Y, A411E, A411V, L413P, G414A, G414M, G414N, G414R, G415R, D416I, D416R, D416Y, K417F, K417R, D418P, D418R, E419M, E419R, E419W, T420K, T420R, W421Q, A422P, A422T, K423D, F424C, F424L, F424N, L426M, T427D, T427F, T427K, T427M, T427Q, T427S, T429D, I430S, N431M, D433G, D433I, D433P, D433Q, D433W, R434N, R434V, N435E, N435L, N435R, A436L, P437Q, P437R, V439C, V439E, V439G, V439K, V439T, I440R, M441A, W442E, W442G, W442R, S443C, S443G, S443Q, S443Y, L444E, L444G, L444V, G445A, G445C, G445V, N446D, N446T, M448C, M448L, M448Q, M448S, M448V, M449D, M449T, M449W, M449Y, E450F, E450S, E450T, E450V, E450W, G451C, G451F, G451L, G451P, G451V, G451W, I452G, I452K, I452M, I452S, I452V, G454L, S455P, S455R, S455W, V456D, V456E, V456F, V456K, S457H, S457T, S457V, G458A, G458P, G458S, G458W, F459N, F459R, F459S, A461M, T462E, T462L, S463V, A464P, A464V, K465F, K465V, K465Y, L466A, L466E, L466P, L466S, L466V, A468D, A468W, T470M, K471F, A472G, A472Y, D474R, S475E, R477L, M479G, M479I, M479R, T480C, T480G, K485E, K485R, K487F, K487N, K487S, K487W, A488H, A488N, A488S, N491W, E492A, S493G, S493L, S493M, S493Q, N494R, T495R, T495W, G497D, D498C, D498S, N499R, N499T, N499Y, L500A, T501M, G504H, G504P, G505D, G505N, G505R, G505S, V506C, V506D, V506E, V506G, V506R, V507L, V507R, G508C, G508E, T509A, T509K, T509M, N510A, N510F, N510I, Y511A, S512F, S512Q, S512T, S512V, S512Y, D513K, D513L, D513P, D513R, G514F, A515C, A515E, A515K, A515R, N516C, N516E, N516Q, N516V, Y517G, D518Y, K519C, K519I, I520C, I520G, I520H, I520M, I520S, I520T, R521N, R521V, T522G, T522N, H524R, H524V, P525T, S526G, W527A, W527H, W527N, W527R, W527S, A528E, Y530M, G531E, G531S, G531T, T534A, A535M, A537D, A537M, A537P, S540E, S540G, G542Q, I543V, N545S, R546L, R546P, R546S, T547A, T547D, T547S, T548E, T548F, T548K, T548P, G549D, G549P, G549W, G550S, A551I, A551Q, S553N, S554N, S554V, D555S, K556W, L558E, L558P, T559Y, Y561R, N563S, S564C, S564F, S564W, A565D, A565E, A565I, A565L, A565R, A565T, G567Q, G567V, A570G, A570K, A570M, A570W, A572S, S573K, S574K, S574Q, S574W, A575D, A575V, W576Y, Y577G, Y577L, D578N, V579G, V579T, V580K, V580S, Q581G, R582L, D583W, F584E, V585I, A586K, G587C, T588D, T588I, Y589V, Y589W, V590H, W591F, T592C, T592L, T592S, F594L, D595E, D595Q, D595S, G598N, P600A, P600E, P600G, P600S, T606S, G607Q, S608V, G609N, G609R, A610M, V611K, W614A, W614P, N619I, N619L, S620G, V625M, V625Y, T627K, T627Q, A628D, F630C, F630D, F630G, F630Y, P631A, P631D, T634V, Y635R, Q639R, D644C, H650E, H650R, W655R, N656V, E657R, A661E, A661K, A661Q, N667L, N667R, P669T, V670C, Y673R, Y673S, T674M, D675P, A676P, A677G, K678T, V679G, V679S, K680I, L681F, T684G, T684V, G687P, S688K, S688L, T689W, E690D, R692G, R692H, I694L, I694W, G695C, G695L, G695W, K697R, S698E, S698L, S698M, T700C, K701A, K701G, K701M, K701S, T704Y, A705E, A705W, Y708F, T709M, Y710D, Y710T, Y710W, Q711Y, V712M, V712Q, E714K, A716R, D717G, D717S, K718A, K718T, D719E, D719T, D719V, T721H, T721L, T721N, T721S, T721V, T721W, H723G, M726D, Y727L, L728S, N731E, N731S, V732R, W734G, W734V, A735Q, A735S, T738S, I739K, S740D, A741I, Y744R, D745F, D745N, N747R, L750S, P752A, P752C, P752Y, G754P, T756N, T756S, E757A, A760N, A769F, A769I, A769N, A769V, A769Y, K770H, L771A, K772A, K772C, K772P, K772V, A773C, A773H, A773M, A773R, A773V, D774A, D774R, A775L, A775W, D776I, D776L, D776S, D776V, R777D, R777E, R777G, R777H, R777P, R777S, K778L, K778N, T781A, T781F, T781G, T781M, T781P, T781Y, A782P, A782Q, D783A, D783C, D783E, D783R, G784L, G784S, G784T, K785S, K785W, K785Y, D786V, L787P, L787T, S788G, S788I, Y789V, I790C, I790R, V792G, D793H, V794C, V794T, V794W, T795P, D796Q, N798I, G799K, G799L, G799M, G799Q, H800A, H800L, V802Y, P803S, D804G, A806I, A806Q, N807W, R808C, R808F, R808N, T810P, T810Q, D812Q, V813T, V813W, K814H, K814L, G815A, G815V, A816C, A816F, K818F, K818L, K818W, K818Y, L819F, V820K, G821E, G821F, G821I, G821N, V822D, V822E, S826G, S826L, P828C, D829S, H830E, H830R, H830V, D831F, D831I, D831P, D831R, D831V, S832R, Y833K, Y833V, A835D, A835E, A835H, A835K, A835W, D836C, D836E, D836H, D836S, D836V, D836Y, N837D, R838G, R838M, R838N, K839A, K839D, A840G, A840P, A840V, F841C, F841W, K844G, V845W, V849L, V849S, Q850C, Q850G, Q850Y, E854C, E857P, I858D, I858Q, I858Y, T859V, V860T, T861W, A862V, K863F, K863N, K863W, D865A, D865G, G866H, L867W, S870E, S870R, V872C, K873G, K873Y, I874G, T877A, G881W, S883L, T884A, K886E, K886V, K886W, T887F, T887G, V888A, Y892P, Y892R, Y893E, N896M, Y897V, K900E, T901G, T901Q, T901R, T901V, T901Y, G902F, G902L, G902R, G902S, G902W, N903D, K904M, K904N, K904S, K904V, P905C, P905R, P905Y, I906W, L907F, P908I, P908L, D910W, E912T, R914A, R914F, R914I, R914K, R914V, R914Y, Y915A, Y915C, Y915G, Y915M, Y915Q, Y915V, S916G, D917C, D917F, D917S, G918E, G918H, G918T, T919D, T919Q, S920E, D921V, R922A, Q923V, T926G, T926R, W927G, W927P, A929P, D933R, Q934S, I935A, I935C, I935E, I935P, I935W, A936I, A936Q, K937G, K937I, K937P, K937Q, A938C, G939D, S940C, S940T, F941C, S942K, S942V, V943A, V943H, V943R, A944D, A944H, A944P, A944R, T946P, T946V, V947G, V947M, V947R, V947T, A948C, A948R, A948W, Q950D, K951S, S953M, S953W, R955C, R955W, V956D, V956Q, M958D, E961D, E961F, E961S, I962C, I962K, I962N, L965C, L965G, L965K, L965M, L965P, L965Y, L966A, L966H, N967M, Y968G, Y968Q, Y968V, S969C, S969I, S969L, S969M, S969Q, A970I, S971V, P973C, P973W, V974T, T976K, T976P, P977C, P977T, A978F, A978M, V979R, L980H, L980N, P981M, P985L, P985W, A986I, A986L, V987A, V987F, V987K, D990W, G991Y, S995L, N997K, A999G, V1000N, D1001L, W1002A, W1002D, W1002E, W1002H, W1002N, W1002P, W1002Q, T1003L, T1003Y, K1004H, P1005Y, A1006C, A1006S, D1007P, N1011T, T1012Q, G1014I, V1018K, T1021F, A1022H, V1024G, V1024H, G1026R, G1026V, K1030D, K1030W, I1035D, Q1041P, V1042N, V1048C, V1048G, V1048M, V1048Q, G1050S, N1051E, N1051K, A1052K, L1053A, Q1057E, Q1057R, N1058V, I1059W, A1061G, D1062L, D1062M, Q1064M, S1065A, D1066M, D1066V, D1066W, T1067M, L1068C, L1068P, A1070P, I1071W, K1072G, K1072Q, K1072S, D1073P, G1074I, G1074L, S1075C, T1082F, G1083E, G1083F, G1083L, G1083S, G1084V, G1085S, P1088E, P1088R, S1089C, S1089G, S1089Q, W1091A, W1091E, W1091G, W1091T, W1091V, W1091Y, T1092G, T1092Q, N1093A, N1093P, N1093Q, W1094D, W1094E, S1097W, K1098D, A1099V, G1100M, H1101V, N1102E, N1102H, T1103E, A1104R, I1106T, T1107S, F1108D, F1108L, E1109A, E1109D, E1109L, E1109W, A1111G, E1113P, E1113V, Q1114E, Q1114R, Q1114S, Q1114T, Q1114V, Q1115A, Q1115K, L1116K, L1116W, G1117I, G1117R, G1117S, G1117T, G1117V, G1117W, I1119N, M1121G, M1121V, R1125V, R1125W, D1126H, S1127I, S1127Q, N1128A, A1129E, A1129Q, A1129V, V1130A, V1130G, V1130R, P1133D, P1133R, D1134L, A1135L, A1135S, A1135W, G1136A, G1136E, G1136P, K1137C, K1137L, K1137Q, K1137R, K1137S, I1140G, I1140L, I1140P, Q1141K, Q1141P, I1142Y, A1144C, A1144N, A1144P, A1144S, A1144V, A1144W, D1145E, D1145R, D1145T, N1148P, W1149C, W1149I, W1149V, T1150K, D1151R, D1151W, L1152W, A1153G, A1154C, E1162A, E1165L, V1167R, K1168W, Y1170E, T1171A, T1171G, A1175N, A1175Q, V1177N, V1177T, G1178M, G1178T, A1179L, A1179W, T1180A, T1180G, T1180I, T1180M, T1180Q, T1180Y, V1184E, V1184L, V1184P, V1184Q, V1184Y, V1186S, N1188W, D1190T, T1191L, T1192H, V1197A, C1199T, A1200W, L1202A, E1204G, K1208W, T1209E, T1209K, A1210D, A1210E, A1210G, A1210K, A1210L, A1210R, A1210T, A1210W, T1211G, T1211K, T1211P, T1211R, T1211S, F1214A, F1214K, F1214S, F1214V, V1215D, V1215L, T1216L, T1216Q, N1217A, N1217S, N1217T, T1218C, T1218E, T1218S, T1218W, S1219A, S1219F, A1220L, L1222A, L1222E, L1222F, L1222V, S1223V, S1224A, S1224G, S1224P, S1224W, L1225E, L1225K, L1225W, T1226P, T1226S, T1226V, N1228D, N1228F, G1229V, T1230I, T1230K, T1230W, K1231F, K1231P, V1232K, V1232R, S1233P, S1233W, S1235W, V1236A, V1236G, V1236P, L1237D, A1238E, A1238R, G1240W, Y1242E, Y1242R, N1243M, N1243T, T1244Q, A1246M, I1248G, A1249H, A1249I, A1249V, D1250I, D1250K, D1250S, D1250T, D1250W, D1250Y, K1252D, G1255H, N1258H, A1259K, T1262R, V1263T, V1263W, P1265R, P1265W, N1269A, N1269K, I1274F, I1274R, T1275W, S1277L, S1277T, S1277W, E1278Q, D1279R, D1279W, H1280E, V1281I, V1281W, K1284G, F1286E, F1286P, T1287C, T1287S, T1287W, I1288A, I1288D, I1288G, I1288K, G1291P, G1291Y, T1292Y, E1295K, F1296A, E1302R, E1302S or D1304V.

93. The lactase variant of the preceding embodiment, wherein the variant has a decreased or an increased galactose inhibition, preferably measured as a galactose inhibition improvement factor (GI-IF) of at least 1.3 or at most 0.7, compared to the lactase of SEQ ID NO: 1.

94. The lactase variant of embodiment 1 which comprises one or more of the following substitutions, wherein each position corresponds to a position in SEQ ID NO: 1: V1H, V1L, E2D, E2V, D3I, D3V, S7N, S7P, M13C, M13R, S14V, S15C, S15I, S15K, S15T, S15V, T16C, P171, V20C, V20M, V20P, Y21C, Y21P, Y21R, Y21T, S22W, A24F, A24L, A24T, A24W, V25Q, D26C, S27A, S27C, S27H, K28C, Q29L, Q29M, Q29R, R31E, R31V, T32S, F35C, W39G, K40C, K40F, K40I, F41A, M42T, L43S, D45P, V47K, V47R, A49D, A49H, A49T, D51I, D51K, D55C, D55H, D55M, D55P, S57C, S57E, S57G, A58Q, A58T, W59I, W59K, W59P, Q60R, Q60S, V62N, V62T, V62W, L64E, H66R, K73D, S75G, S75L, S75R, S75V, Q76G, Q76I, S77D, S77E, S77G, S77H, S77K, S77R, N78C, E79H, E79S, E79T, A83T, L85A, L85F, L85N, L85S, L85V, L85W, P86Q, G87D, G88F, G90T, W91P, W91Y, Y92H, Y92I, Y92M, Y92S, Y92T, Y92V, R93A, R93D, R93F, R93H, R93I, R93L, R93N, R93T, R93V, R93Y, K94A, K94S, K94T, S95C, S95D, S95G, S951, F96A, F96C, F96I, F96K, F96M, F96P, F96V, T97F, I98C, I98H, R100T, D101A, D101P, L102P, R106W, I107S, A108E, I109M, N110A, N110S, N110T, N110W, F111A, F111L, V114R, Y115E, M116C, A118P, V120A, V120K, W121R, W121V, H130A, H130T, Y132E, F137L, T142S, G149M, E150L, V154K, V154L, V157P, V157S, E158H, N159T, L161K, L161M, L161S, L161W, P162N, P162T, P162W, S164A, R165H, W166S, Y167A, S170Q, I172K, I172P, Y173H, R174E, V176E, V176K, T177K, L178I, L178Q, T179C, T179I, T179N, V180D, V180G, T181A, V184R, H185G, H185L, G187D, N188V, G190F, K194I, S197C, A199P, Q201E, N202Q, N202S, N202W, D217L, D217M, T218G, A220G, A222R, N223S, Q228R, K236M, T237D, G242K, I251Y, A252W, A252Y, S261H, A266D, S267V, P268R, S272T, S272W, V281Q, V291P, L292V, D293C, D293W, Y295F, D296F, D296K, E298I, E298R, F301C, W303A, W303C, W303D, T304D, T304K, T304P, G305N, G305P, T309C, T309D, T309E, S310H, S310L, S310N, F312H, L314A, L314V, N315S, K318N, K320V, K320W, L321M, K322I, V324T, S325G, M326T, H328C, H328F, H328G, H328M, H328T, G331V, A335G, A335L, A337G, A337L, R340A, R340L, A341M, I342G, E343N, E343Y, R344G, Q345S, V346A, E347A, I348D, I348M, Q350G, M352T, G353K, I357T, T359E, T359L, T360V, N362S, N362T, P363A, P363I, P363L, P363M, A364I, A364M, A364P, A365C, A365I, A365P, A365V, K366L, K366M, K366S, L368A, L368Q, L368S, I369E, I369G, I369K, I369V, I369W, D370L, V371F, V371Q, C372P, N373G, N373L, G376A, G376S, V377M, V377T, V379A, V379N, V380P, E381A, E381G, E381Q, E381T, V383A, M386G, M386N, M386S, W387H, N388L, R389A, R389E, R389Q, R389S, R389T, S390C, S390D, S390P, S390T, G393E, G393R, G393V, T395C, E396K, E396L, E396V, E396W, Y398M, G399S, K400A, K400C, K400N, K400V, W401F, W401K, F402W, F402Y, G403A, G403K, G403Q, G403V, Q404F, Q404L, Q404M, Q404P, Q404V, A405C, A405E, A405K, A405R, A405T, I406D, I406N, A407C, A407G, A407Q, N410Y, A411E, G414M, G414N, K417F, K417R, A422P, T427D, T427F, T427Q, T427S, T429D, N431M, D433G, D433I, D433P, D433Q, D433W, R434N, R434V, N435E, N435L, A436L, V439C, V439K, W442E, W442G, S443C, S443G, S443Q, L444E, L444V, N446D, M448C, M448L, M449D, M449T, M449W, M449Y, E450F, E450S, E450T, E450V, E450W, G451C, G451F, G451L, G451P, G451V, G451W, I452K, I452S, I452V, G454L, S455P, S455R, V456D, V456E, V456F, V456K, S457H, S457T, S457V, G458A, G458P, G458S, F459R, A461M, T462E, T462L, K465V, K465Y, L466P, A468D, K471F, A472G, A472Y, S475E, R477L, M479G, K485E, K485R, K487F, K487S, K487W, S493G, S493L, S493Q, N494R, G497D, N499T, G505D, G505R, G505S, V506C, V506E, V506R, V507R, G508C, G508E, T509A, T509K, T509M, N510A, N510F, N510I, Y511A, S512F, S512Q, S512T, S512V, S512Y, D513P, D513R, A515E, A515P, N516C, N516E, N516Q, N516V, Y517G, D518Y, K519C, K519I, I520C, I520G, I520H, I520M, I520S, I520T, R521V, H524R, H524V, S526G, W527A, W527H, W527N, W527R, W527S, A528E, A537M, A537P, S540E, G549D, G549P, G549W, L558E, L558P, T559Y, S564C, S564F, S564W, A565D, A565E, A565I, A565L, A565R, A565T, G567Q, G567V, A570G, A575D, R582L, D583W, W591F, T592L, G598N, P600A, P600E, P600G, P600S, G607Q, W614A, N619I, N619L, V625M, V625Y, T627K, T627Q, A628D, F630C, F630Y, P631D, T634V, Q639R, W655R, E657R, A661E, A661K, A661Q, N667L, N667R, P669T, Y673S, T674M, A676P, V679S, K680I, G687P, S688K, S688L, T689W, E690D, R692G, R692H, I694L, I694W, G695C, G695L, G695W, K697R, S698E, S698M, K701A, K701G, K701M, K701S, A705W, Y708F, Y710T, V712M, V712Q, E714K, A716R, D717G, D717S, K718A, K718T, D719E, D719T, D719V, T721H, T721L, T721N, T721S, T721V, T721W, H723G, M726D, Y727L, L728S, N731E, V732R, W734G, A735Q, A735S, T738S, I739K, S740D, A741I, Y744R, D745N, L750S, P752C, P752Y, T756N, E757A, A769F, A769I, A769N, A769V, A769Y, K770H, L771A, K772A, K772C, K772P, A773C, A773M, A773R, D774A, D776V, R777H, R777S, K778N, T781A, T781F, T781G, T781P, T781Y, A782Q, D783A, K785S, K785W, K785Y, D786V, Y789V, I790R, N798I, G799K, G799L, V802Y, P803S, A806I, A806Q, R808F, T810P, T810Q, V813W, K818F, K818L, K818W, K818Y, L819F, G821I, V822D, V822E, S826G, D829S, D831P, S832R, A835D, A835H, A835K, D836C, D836E, D836S, D836V, D836Y, R838N, K839A, K839D, A840G, A840P, A840V, E854C, I858D, T861W, D865A, D865G, G866H, L867W, S870E, V872C, K873G, K873Y, I874G, S883L, K886V, K886W, T887G, V888A, Y892P, Y893E, Y897V, K900E, T901Q, T901R, T901Y, G902F, G902R, G902S, G902W, N903D, K904M, K904N, K904S, K904V, P905C, P905R, P905Y, I906W, L907F, P908I, P908L, D910W, E912T, R914A, R914F, R914I, R914K, R914V, R914Y, Y915A, Y915C, Y915G, Y915M, Y915Q, Y915V, S916G, D917C, D917F, D917S, G918E, G918H, G918T, T919D, T919Q, D921V, R922A, Q923V, T926R, W927P, A929P, D933R, Q934S, I935A, I935C, I935E, I935P, I935W, A936I, A936Q, K937G, K937I, K937P, K937Q, A938C, G939D, S940C, F941C, S942K, S942V, V943A, T946P, V947G, V947R, V947T, A948W, K951S, S953M, R955C, R955W, V956D, V956Q, E961D, E961F, E961S, I962C, I962K, I962N, L965C, L965G, L965K, L965M, L965P, L965Y, L966A, N967M, Y968G, Y968Q, Y968V, S969C, S969I, S969L, S969M, S969Q, A970I, S971V, P973C, P973W, V974T, T976K, T976P, P977C, P977T, A978F, A978M, L980H, L980N, P981M, P985L, P985W, A986I, A986L, V987A, V987F, V987K, D990W, G991Y, S995L, N997K, D1001L, W1002A, W1002D, W1002E, W1002H, W1002N, W1002P, W1002Q, T1003Y, P1005Y, A1006C, A1006S, D1007P, N1011T, T1012Q, G1014I, V1018K, A1022H, V1024G, V1024H, G1026V, K1030D, K1030W, I1035D, Q1041P, V1042N, V1048C, V1048G, V1048M, V1048Q, N1051E, N1051K, A1052K, L1053A, Q1057E, A1061G, D1062M, Q1064M, S1065A, D1066V, T1067M, L1068P, K1072S, G1074L, T1082F, G1083E, G1083F, G1083L, G1084V, G1085S, P1088E, P1088R, S1089G, S1089Q, W1091Y, N1093A, N1093P, N1093Q, W1094D, W1094E, K1098D, A1099V, H1101V, N1102H, T1103E, E1109A, E1109D, E1109L, E1109W, A1111G, E1113P, E1113V, Q1114E, Q1114R, Q1114S, Q1114T, Q1114V, Q1115A, Q1115K, L1116K, L1116W, G1117I, G1117R, G1117S, G1117T, G1117V, G1117W, I1119N, M1121G, R1125V, R1125W, S1127I, S1127Q, N1128A, A1129E, V1130A, V1130R, P1133D, P1133R, A1135L, A1135W, G1136P, K1137R, K1137S, I1140P, Q1141K, I1142Y, A1144N, A1144P, A1144S, A1144V, A1144W, D1145E, D1145R, D1145T, N1148P, W1149C, W1149V, T1150K, D1151W, L1152W, A1153G, E1162A, V1167R, G1178M, G1178T, A1179L, A1179W, T1180A, T1180G, T1180I, T1180M, T1180Q, T1180Y, V1184E, V1184L, V1184P, V1184Q, V1184Y, V1186S, N1188W, D1190T, T1191L, V1197A, E1204G, K1208W, T1209E, T1209K, A1210D, A1210G, A1210K, A1210L, A1210R, A1210T, A1210W, T1211G, T1211K, T1211P, T1211R, T1211S, F1214A, F1214K, F1214S, F1214V, V1215D, V1215L, T1216L, T1216Q, N1217A, N1217S, N1217T, T1218C, T1218E, T1218S, T1218W, L1222A, L1222E, L1222F, L1222V, S1224P, S1224W, L1225E, L1225K, T1226P, G1229V, T1230K, T1230W, V1232R, S1235W, V1236A, V1236G, V1236P, A1238E, A1238R, G1255H, A1259K, T1262R, V1263T, P1265W, I1274F, I1274R, T1275W, S1277L, S1277T, S1277W, E1278Q, D1279R, D1279W, H1280E, V1281I, V1281W, K1284G, F1286E, F1286P, T1287C, T1287S, T1287W, I1288A, I1288D, I1288G, I1288K, E1295K, F1296A, E1302R, E1302S or D1304V.

95. The lactase variant of the preceding embodiment, wherein the variant has a decreased galactose inhibition, preferably measured as a galactose inhibition improvement factor (GI-IF) of at least 1.3, compared to the lactase of SEQ ID NO: 1.

96. The lactase variant of embodiment 1 which comprises one or more of the following substitutions, wherein each position corresponds to a position in SEQ ID NO: 1: V1H, V1L, E2D, E2V, D3I, D3V, S7N, S7P, M13C, M13R, S14V, S15C, S15I, S15K, S15T, S15V, T16C, P171, V20C, V20M, V20P, Y21C, Y21P, Y21R, Y21T, S22W, A24F, A24L, A24T, A24W, V25Q, D26C, S27A, S27C, S27H, K28C, Q29L, Q29M, Q29R, R31E, R31V, T32S, F35C, W39G, K40C, K40F, K40I, F41A, M42T, L43S, D45P, V47K, V47R, A49D, A49H, A49T, D51I, D51K, D55C, D55H, D55M, D55P, S57C, S57E, S57G, A58Q, A58T, W59I, W59K, W59P, Q60R, Q60S, V62N, V62T, V62W, L64E, H66R, K73D, S75G, S75L, S75R, S75V, Q76G, Q76I, S77D, S77E, S77G, S77H, S77K, S77R, N78C, E79H, E79S, E79T, A83T, L85A, L85F, L85N, L85S, L85V, L85W, P86Q, G87D, G88F, G90T, W91P, W91Y, Y92H, Y92I, Y92M, Y92S, Y92T, Y92V, R93A, R93D, R93F, R93H, R93I, R93L, R93N, R93T, R93V, R93Y, K94A, K94S, K94T, S95C, S95D, S95G, S951, F96A, F96C, F96I, F96K, F96M, F96P, F96V, T97F, I98C, I98H, R100T, D101A, D101P, L102P, R106W, I107S, A108E, I109M, N110A, N110S, N110T, N110W, F111A, F111L, V114R, Y115E, M116C, A118P, V120A, V120K, W121R, W121V, H130A, H130T, Y132E, F137L, T142S, G149M, E150L, V154K, V154L, V157P, V157S, E158H, N159T, L161K, L161M, L161S, L161W, P162N, P162T, P162W, S164A, R165H, W166S, Y167A, S170Q, I172K, I172P, Y173H, R174E, V176E, V176K, T177K, L178I, L178Q, T179C, T179I, T179N, V180D, V180G, T181A, V184R, H185G, H185L, G187D, N188V, G190F, K194I, S197C, A199P, Q201E, N202Q, N202S, N202W, D217L, D217M, T218G, A220G, A222R, N223S, Q228R, K236M, T237D, G242K, I251Y, A252W, A252Y, S261H, A266D, S267V, P268R, S272T, S272W, V281Q, V291P, L292V, D293C, D293W, Y295F, D296F, D296K, E298I, E298R, F301C, W303A, W303C, W303D, T304D, T304K, T304P, G305N, G305P, T309C, T309D, T309E, S310H, S310L, S310N, F312H, L314A, L314V, N315S, K318N, K320V, K320W, L321M, K322I, V324T, S325G, M326T, H328C, H328F, H328G, H328M, H328T, G331V, A335G, A335L, A337G, A337L, R340A, R340L, A341M, I342G, E343N, E343Y, R344G, Q345S, V346A, E347A, I348D, I348M, Q350G, M352T, G353K, I357T, T359E, T359L, T360V, N362S, N362T, P363A, P363I, P363L, P363M, A364I, A364M, A364P, A365C, A365I, A365P, A365V, K366L, K366M, K366S, L368A, L368Q, L368S, I369E, I369G, I369K, I369V, I369W, D370L, V371F, V371Q, C372P, N373G, N373L, G376A, G376S, V377M, V377T, V379A, V379N, V380P, E381A, E381G, E381Q, E381T, V383A, M386G, M386N, M386S, W387H, N388L, R389A, R389E, R389Q, R389S, R389T, S390C, S390D, S390P, S390T, G393E, G393R, G393V, T395C, E396K, E396L, E396V, E396W, Y398M, G399S, K400A, K400C, K400N, K400V, W401F, W401K, F402W, F402Y, G403A, G403K, G403Q, G403V, Q404F, Q404L, Q404M, Q404P, Q404V, A405C, A405E, A405K, A405R, I406D, I406N, A407C, A407G, A407Q, N410Y, A411E, G414M, G414N, K417F, K417R, A422P, T427D, T427F, T427Q, T427S, T429D, N431M, D433G, D433I, D433P, D433Q, D433W, R434N, R434V, N435E, N435L, A436L, V439C, V439K, W442E, W442G, S443C, S443G, S443Q, L444E, L444V, N446D, M448C, M448L, M449D, M449T, M449W, M449Y, E450F, E450S, E450T, E450V, E450W, G451C, G451F, G451L, G451P, G451V, G451W, I452K, I452S, I452V, G454L, S455P, S455R, V456D, V456E, V456F, V456K, S457H, S457T, S457V, G458A, G458P, G458S, F459R, A461M, T462E, T462L, K465V, K465Y, L466P, A468D, K471F, A472G, A472Y, S475E, R477L, M479G, K485E, K485R, K487F, K487S, K487W, S493G, S493L, S493Q, N494R, G497D, N499T, G505D, G505R, G505S, V506C, V506E, V506R, V507R, G508C, G508E, T509A, T509K, T509M, N510A, N510F, N510I, Y511A, S512F, S512Q, S512T, S512V, S512Y, D513P, D513R, A515E, N516C, N516E, N516Q, N516V, Y517G, D518Y, K519C, K519I, I520C, I520G, I520H, I520M, I520S, I520T, R521V, H524R, H524V, S526G, W527A, W527H, W527N, W527R, W527S, A528E, A537M, A537P, S540E, G549D, G549P, G549W, L558E, L558P, T559Y, S564C, S564F, S564W, A565D, A565E, A565I, A565L, A565R, A565T, G567Q, G567V, A570G, A575D, R582L, D583W, W591F, T592L, G598N, P600A, P600E, P600G, P600S, G607Q, W614A, N619I, N619L, V625M, V625Y, T627K, T627Q, A628D, F630C, F630Y, P631D, T634V, Q639R, W655R, E657R, A661E, A661K, A661Q, N667L, N667R, P669T, Y673S, T674M, A676P, V679S, K680I, G687P, S688K, S688L, T689W, E690D, R692G, R692H, I694L, I694W, G695C, G695L, G695W, K697R, S698E, S698M, K701A, K701G, K701M, K701S, A705W, Y708F, Y710T, V712M, V712Q, E714K, A716R, D717G, D717S, K718A, K718T, D719E, D719T, D719V, T721H, T721L, T721N, T721S, T721V, T721W, H723G, M726D, Y727L, L728S, N731E, V732R, W734G, A735Q, A735S, T738S, I739K, S740D, A741I, Y744R, D745N, L750S, P752C, P752Y, T756N, E757A, A769F, A769I, A769N, A769V, A769Y, K770H, L771A, K772A, K772C, K772P, A773C, A773M, A773R, D774A, D776V, R777H, R777S, K778N, T781A, T781F, T781G, T781P, T781Y, A782Q, D783A, K785S, K785W, K785Y, D786V, Y789V, I790R, N798I, G799K, G799L, V802Y, P803S, A806I, A806Q, R808F, T810P, T810Q, V813W, K818F, K818L, K818W, K818Y, L819F, G821I, V822D, V822E, S826G, D829S, D831P, S832R, A835D, A835H, A835K, D836C, D836E, D836S, D836V, D836Y, R838N, K839A, K839D, A840G, A840P, A840V, E854C, I858D, T861W, D865A, D865G, G866H, L867W, S870E, V872C, K873G, K873Y, I874G, S883L, K886V, K886W, T887G, V888A, Y892P, Y893E, Y897V, K900E, T901Q, T901R, T901Y, G902F, G902R, G902S, G902W, N903D, K904M, K904N, K904S, K904V, P905C, P905R, P905Y, I906W, L907F, P908I, P908L, D910W, E912T, R914A, R914F, R914I, R914K, R914V, R914Y, Y915A, Y915C, Y915G, Y915M, Y915Q, Y915V, S916G, D917C, D917F, D917S, G918E, G918H, G918T, T919D, T919Q, D921V, R922A, Q923V, T926R, W927P, A929P, D933R, Q934S, I935A, I935C, I935E, I935P, I935W, A936I, A936Q, K937G, K937I, K937P, K937Q, A938C, G939D, S940C, F941C, S942K, S942V, V943A, T946P, V947G, V947R, V947T, A948W, K951S, S953M, R955C, R955W, V956D, V956Q, E961D, E961F, E961S, I962C, I962K, I962N, L965C, L965G, L965K, L965M, L965P, L965Y, L966A, N967M, Y968G, Y968Q, Y968V, S969C, S969I, S969L, S969M, S969Q, A970I, S971V, P973C, P973W, V974T, T976K, T976P, P977C, P977T, A978F, A978M, L980H, L980N, P981M, P985L, P985W, A986I, A986L, V987A, V987F, V987K, D990W, G991Y, S995L, N997K, D1001L, W1002A, W1002D, W1002E, W1002H, W1002N, W1002P, W1002Q, T1003Y, P1005Y, A1006C, A1006S, D1007P, N1011T, T1012Q, G1014I, V1018K, A1022H, V1024G, V1024H, G1026V, K1030D, K1030W, I1035D, Q1041P, V1042N, V1048C, V1048G, V1048M, V1048Q, N1051E, N1051K, A1052K, L1053A, Q1057E, A1061G, D1062M, Q1064M, S1065A, D1066V, T1067M, L1068P, K1072S, G1074L, T1082F, G1083E, G1083F, G1083L, G1084V, G1085S, P1088E, P1088R, S1089G, S1089Q, W1091Y, N1093A, N1093P, N1093Q, W1094D, W1094E, K1098D, A1099V, H1101V, N1102H, T1103E, E1109A, E1109D, E1109L, E1109W, A1111G, E1113P, E1113V, Q1114E, Q1114R, Q1114S, Q1114T, Q1114V, Q1115A, Q1115K, L1116K, L1116W, G1117I, G1117R, G1117S, G1117T, G1117V, G1117W, I1119N, M1121G, R1125V, R1125W, S1127I, S1127Q, N1128A, A1129E, V1130A, V1130R, P1133D, P1133R, A1135L, A1135W, G1136P, K1137R, K1137S, I1140P, Q1141K, I1142Y, A1144N, A1144P, A1144S, A1144V, A1144W, D1145E, D1145R, D1145T, N1148P, W1149C, W1149V, T1150K, D1151W, L1152W, A1153G, E1162A, V1167R, G1178M, G1178T, A1179L, A1179W, T1180A, T1180G, T1180I, T1180M, T1180Q, T1180Y, V1184E, V1184L, V1184P, V1184Q, V1184Y, V1186S, N1188W, D1190T, T1191L, V1197A, E1204G, K1208W, T1209E, T1209K, A1210D, A1210G, A1210K, A1210L, A1210R, A1210T, A1210W, T1211G, T1211K, T1211P, T1211R, T1211S, F1214A, F1214K, F1214S, F1214V, V1215D, V1215L, T1216L, T1216Q, N1217A, N1217S, N1217T, T1218C, T1218E, T1218S, T1218W, L1222A, L1222E, L1222F, L1222V, S1224P, S1224W, L1225E, L1225K, T1226P, G1229V, T1230K, T1230W, V1232R, S1235W, V1236A, V1236G, V1236P, A1238E, A1238R, G1255H, A1259K, T1262R, V1263T, P1265W, I1274F, I1274R, T1275W, S1277L, S1277T, S1277W, E1278Q, D1279R, D1279W, H1280E, V1281I, V1281W, K1284G, F1286E, F1286P, T1287C, T1287S, T1287W, I1288A, I1288D, I1288G, I1288K, E1295K, F1296A, E1302R, E1302S or D1304V.

97. The lactase variant of the preceding embodiment, wherein the variant has a decreased galactose inhibition, preferably measured as a galactose inhibition improvement factor (GI-IF) of at least 1.3, compared to the lactase of SEQ ID NO: 1.

98. The lactase variant of embodiment 1 which comprises one or more of the following substitutions, wherein each position corresponds to a position in SEQ ID NO: 1: E2V, D3I, D3S, D3V, S7P, M13R, S15C, S15I, S15K, S15Y, T16C, V20P, Y21C, Y21R, Y21T, S23L, S23M, A24F, A24T, D26C, S27A, K28C, Q29L, Q29M, Q29R, R31E, R31V, T32S, D34S, F35E, K40F, K40I, M42E, L43T, D45P, V47K, V47R, A49D, A49H, A49R, A49T, D51I, D51K, D51P, A53S, D55H, D55M, S57C, S57E, S57G, A58T, W59P, V62N, L64E, H66Y, K73D, S75G, S75L, S75R, Q76G, S77E, S77F, S77H, S77K, N78C, E79H, E79S, E79T, E81A, E81Q, L85F, L85S, L85V, L85W, P86Q, G87D, G88F, G88Q, G90T, W91Y, Y92H, Y92M, Y92S, Y92V, R93A, R93D, R93F, R93H, R93L, R93N, R93T, R93V, R93Y, K94S, S95C, S95G, S95I, F96A, F96C, F96I, F96S, F96V, T97F, I98H, R100T, D101A, D101P, L102P, I109M, N110T, F111A, F111L, D112T, V114R, Y115E, V120A, W121R, W121V, G124E, H130A, H130T, Y132E, P136R, F137L, K146A, G149M, E150L, I153A, V157P, E158G, E158H, N159T, L161K, L161S, L161W, P162N, P162T, P162W, R165H, W166S, Y167A, S170Q, I172P, R174E, V176E, V176K, T177K, L178I, L178Q, T179C, V180G, H185G, H185L, N188V, N189E, G190C, S197C, D217L, D217M, T218G, A220G, A220V, A222R, N223S, Q228R, K233P, K236M, T237D, A239G, A239T, G242Y, A247P, A247R, S248E, K249G, K249Y, I251Y, A252F, A252W, A252Y, G254I, G254R, A255K, S256Y, S261R, T262D, T264M, P268R, S272W, L278V, Y279W, L286F, V291D, V291P, L292V, D293C, D296F, E298I, E298R, F301C, W303A, T304P, G305N, G305P, G305W, T309D, F312H, K320V, K320W, S325G, M326E, M326G, M326T, M326Y, H328C, H328M, H328T, A337G, A337L, R340A, A341M, I342K, E343N, Q345G, Q345K, Q345S, I348D, Q350G, T359E, T360V, N362S, N362T, P363I, P363L, P363M, A364I, A364M, A364P, A365C, L368E, L368Q, L368S, I369G, I369K, I369W, D370L, N373G, N373L, G376A, G376S, V377M, V379A, V380P, E381A, E381T, M386G, M386N, M386S, W387L, N388L, N388R, R389A, R389E, R389Q, R389S, R389T, S390C, S390D, S390G, S390P, S390Q, S390T, K391E, N392D, G393R, G393V, N394L, T395C, T395H, T395N, T395S, T395W, E396K, E396L, E396V, E396W, Y398N, G399S, K400A, K400C, K400N, K400P, K400S, K400V, W401F, W401K, F402W, F402Y, G403A, G403K, G403V, Q404F, Q404H, Q404L, Q404M, Q404P, Q404V, A405C, A405E, A405H, A405R, A405T, I406D, I406N, G408W, D409N, A411E, L413P, G414A, G414R, D416I, D416Y, K417F, D418P, D418R, E419M, E419R, T420K, T420R, K423D, F424C, F424L, L426M, T427D, T427Q, T427S, T429D, N431M, D433P, D433W, R434N, R434V, N435L, A436L, V439C, V439G, V439K, V439T, W442E, W442G, W442R, S443C, S443Y, L444V, G445A, G445C, G445V, M448C, M448L, M448S, M449D, M449T, M449W, M449Y, E450F, E450S, E450T, E450V, E450W, G451C, G451F, G451L, G451P, G451V, G451W, I452K, G454L, S455P, S455R, V456D, V456E, V456F, V456K, S457T, S457V, G458A, G458P, F459R, F459S, K465Y, L466P, L466V, A468D, A472G, A472Y, S475E, R477L, M479G, M479R, K485E, K487F, K487N, A488N, N491W, S493M, S493Q, T495W, D498C, N499R, N499T, N499Y, L500A, G504H, G505D, G505S, V506C, V506E, V506R, V507L, V507R, G508C, G508E, T509K, T509M, N510A, N510F, N510I, Y511A, S512F, S512T, S512Y, D513L, D513P, D513R, G514F, A515E, N516C, Y517G, D518Y, K519C, K519I, I520G, I520H, I520S, R521N, R521V, T522N, H524R, H524V, P525T, S526G, W527N, W527R, W527S, A528E, G531T, A537P, S540E, R546L, R546P, R546S, T547A, T547S, T548F, T548K, T548P, G549P, G549W, G550S, A551Q, S553N, S554N, D555S, K556W, L558P, T559Y, S564C, S564F, A565E, A565I, A565R, G567Q, G567V, A570K, A570M, A572S, S574W, W576Y, Y577L, V579G, R582L, D583W, V585I, A586K, T588D, T592C, T592L, T592S, F594L, D595Q, P600A, P600E, P600G, P600S, G607Q, S608V, G609N, G609R, A610M, V611K, W614A, N619L, S620G, V625M, V625Y, F630G, T634V, Y635R, Q639R, D644C, H650R, W655R, N656V, A661E, P669T, V670C, Y673S, A676P, K680I, T684V, G687P, S688L, R692H, I694L, I694W, G695C, G695L, G695W, K697R, S698E, T700C, K701A, K701G, K701M, A705W, T709M, Y710T, V712M, E714K, A716R, D717G, D717S, K718A, D719T, D719V, T721N, T721S, T721W, H723G, M726D, L728S, N731S, W734G, W734V, A735Q, A735S, I739K, S740D, A741I, D745F, L750S, P752A, P752C, P752Y, G754P, T756N, T756S, E757A, A760N, A769F, A769I, A769N, A769V, A769Y, K770H, K772A, K772C, K772P, A773C, A773H, A773V, D774A, A775L, A775W, D776I, D776L, D776S, R777D, R777E, R777G, R777H, R777P, R777S, T781A, T781G, T781P, T781Y, A782Q, D783C, D783E, D783R, G784L, G784S, K785S, K785W, D786V, L787P, L787T, Y789V, V792G, D793H, V794T, T795P, N798I, G799K, G799L, G799M, G799Q, H800A, H800L, P803S, A806Q, R808C, T810P, V813T, K814H, G815A, A816C, A816F, K818F, K818L, K818W, G821E, G821I, V822D, V822E, S826L, H830R, D831I, D831P, D831V, S832R, A835D, A835H, A835W, D836E, D836H, D836S, D836Y, R838G, K839D, A840G, K844G, Q850C, Q850G, E854C, I858D, I858Q, I858Y, K863F, K863N, D865A, D865G, L867W, S870R, V872C, I874G, S883L, T887F, T887G, V888A, Y892P, Y892R, Y893E, T901V, T901Y, G902R, G902S, G902W, N903D, K904M, K904V, P905R, P905Y, I906W, P908I, D910W, E912T, R914A, R914F, R914K, R914Y, Y915A, Y915C, Y915M, Y915V, S916G, D917C, D917F, D917S, G918H, T919Q, D921V, R922A, Q923V, W927G, Q934S, I935P, A936Q, K937I, K937P, A938C, S940T, S942K, S942V, V943A, V943H, V943R, A944P, T946P, T946V, V947G, A948C, Q950D, V956Q, E961D, E961F, E961S, I962C, I962N, L965C, L965G, L965M, L965P, L965Y, L966A, Y968G, Y968V, S969C, S969I, S969M, P973C, T976K, T976P, P977T, A978M, V979R, L980H, L980N, P981M, P985L, P985W, A986I, V987K, D990W, S995L, N997K, D1001L, W1002A, W1002D, W1002H, W1002N, W1002P, T1003Y, K1004H, A1006S, D1007P, V1018K, A1022H, V1024G, G1026R, K1030W, I1035D, V1048M, V1048Q, G1050S, N1051E, A1052K, L1053A, Q1057E, Q1057R, D1066W, L1068C, I1071W, K1072G, K1072Q, T1082F, G1083E, G1084V, G1085S, P1088E, S1089Q, W1091A, W1091V, T1092Q, N1093P, N1093Q, W1094D, W1094E, S1097W, A1099V, N1102E, N1102H, A1104R, T1107S, E1109D, E1109L, E1109W, A1111G, E1113P, Q1114S, Q1114V, Q1115A, Q1115K, L1116K, L1116W, G1117I, G1117S, G1117T, G1117V, G1117W, I1119N, R1125V, R1125W, S1127Q, N1128A, A1129E, V1130A, V1130G, V1130R, P1133D, P1133R, G1136E, K1137R, K1137S, Q1141P, A1144P, A1144S, D1145E, D1145R, D1145T, T1150K, D1151R, D1151W, V1167R, A1175N, G1178M, A1179W, T1180A, T1180G, T1180I, T1180M, T1180Q, T1180Y, V1184E, V1184L, V1184P, V1186S, N1188W, C1199T, K1208W, T1209E, T1209K, A1210D, A1210G, A1210R, A1210T, A1210W, T1211G, T1211P, T1211R, F1214A, F1214K, F1214V, V1215D, T1216L, T1216Q, N1217A, T1218W, S1219A, S1219F, L1222F, L1222V, S1223V, S1224P, S1224W, L1225E, L1225K, T1226P, N1228F, G1229V, T1230K, T1230W, K1231F, S1233P, A1238E, A1238R, N1243T, T1244Q, A1246M, D1250I, D1250S, D1250W, A1259K, P1265W, N1269A, N1269K, I1274F, I1274R, T1275W, S1277W, E1278Q, D1279R, D1279W, H1280E, V1281I, V1281W, K1284G, F1286E, T1287C, T1287W, I1288A, I1288D, I1288G, I1288K, E1302R, E1302S or D1304V.

99. The lactase variant of the preceding embodiment, wherein the variant has a decreased or an increased galactose inhibition, preferably measured as a galactose inhibition improvement factor (GI-IF) of at least 1.4 or at most 0.6, compared to the lactase of SEQ ID NO: 1.

100. The lactase variant of embodiment 1 which comprises one or more of the following substitutions, wherein each position corresponds to a position in SEQ ID NO: 1: E2V, D3I, D3S, D3V, S7P, M13R, S15C, S15I, S15K, S15Y, T16C, V20P, Y21C, Y21R, Y21T, S23L, S23M, A24F, A24T, D26C, S27A, K28C, Q29L, Q29M, Q29R, R31E, R31V, T32S, D34S, F35E, K40F, K40I, M42E, L43T, D45P, V47K, V47R, A49D, A49H, A49R, A49T, D51I, D51K, D51P, A53S, D55H, D55M, S57C, S57E, S57G, A58T, W59P, V62N, L64E, H66Y, K73D, S75G, S75L, S75R, Q76G, S77E, S77F, S77H, S77K, N78C, E79H, E79S, E79T, E81A, E81Q, L85F, L85S, L85V, L85W, P86Q, G87D, G88F, G88Q, G90T, W91Y, Y92H, Y92M, Y92S, Y92V, R93A, R93D, R93F, R93H, R93L, R93N, R93T, R93V, R93Y, K94S, S95C, S95G, S95I, F96A, F96C, F96I, F96S, F96V, T97F, I98H, R100T, D101A, D101P, L102P, I109M, N110T, F111A, F111L, D112T, V114R, Y115E, V120A, W121R, W121V, G124E, H130A, H130T, Y132E, P136R, F137L, K146A, G149M, E150L, I153A, V157P, E158G, E158H, N159T, L161K, L161S, L161W, P162N, P162T, P162W, R165H, W166S, Y167A, S170Q, I172P, R174E, V176E, V176K, T177K, L178I, L178Q, T179C, V180G, H185G, H185L, N188V, N189E, G190C, S197C, D217L, D217M, T218G, A220G, A220V, A222R, N223S, Q228R, K233P, K236M, T237D, A239G, A239T, G242Y, A247P, A247R, S248E, K249G, K249Y, I251Y, A252F, A252W, A252Y, G254I, G254R, A255K, S256Y, S261R, T262D, T264M, P268R, S272W, L278V, Y279W, L286F, V291D, V291P, L292V, D293C, D296F, E298I, E298R, F301C, W303A, T304P, G305N, G305P, G305W, T309D, F312H, K320V, K320W, S325G, M326E, M326G, M326T, M326Y, H328C, H328M, H328T, A337G, A337L, R340A, A341M, I342K, E343N, Q345G, Q345K, Q345S, I348D, Q350G, T359E, T360V, N362S, N362T, P363I, P363L, P363M, A364I, A364M, A364P, A365C, L368E, L368Q, L368S, I369G, I369K, I369W, D370L, N373G, N373L, G376A, G376S, V377M, V379A, V380P, E381A, E381T, M386G, M386N, M386S, W387L, N388L, N388R, R389A, R389E, R389Q, R389S, R389T, S390C, S390D, S390G, S390P, S390Q, S390T, K391E, N392D, G393R, G393V, N394L, T395C, T395H, T395N, T395S, T395W, E396K, E396L, E396V, E396W, Y398N, G399S, K400A, K400C, K400N, K400P, K400S, K400V, W401F, W401K, F402W, F402Y, G403A, G403K, G403V, Q404F, Q404H, Q404L, Q404M, Q404P, Q404V, A405C, A405E, A405H, A405R, I406D, I406N, G408W, D409N, A411E, L413P, G414A, G414R, D416I, D416Y, K417F, D418P, D418R, E419M, E419R, T420K, T420R, K423D, F424C, F424L, L426M, T427D, T427Q, T427S, T429D, N431M, D433P, D433W, R434N, R434V, N435L, A436L, V439C, V439G, V439K, V439T, W442E, W442G, W442R, S443C, S443Y, L444V, G445A, G445C, G445V, M448C, M448L, M448S, M449D, M449T, M449W, M449Y, E450F, E450S, E450T, E450V, E450W, G451C, G451F, G451L, G451P, G451V, G451W, I452K, G454L, S455P, S455R, V456D, V456E, V456F, V456K, S457T, S457V, G458A, G458P, F459R, F459S, K465Y, L466P, L466V, A468D, A472G, A472Y, S475E, R477L, M479G, M479R, K485E, K487F, K487N, A488N, N491W, S493M, S493Q, T495W, D498C, N499R, N499T, N499Y, L500A, G504H, G505D, G505S, V506C, V506E, V506R, V507L, V507R, G508C, G508E, T509K, T509M, N510A, N510F, N510I, Y511A, S512F, S512T, S512Y, D513L, D513P, D513R, G514F, A515E, N516C, Y517G, D518Y, K519C, K519I, I520G, I520H, I520S, R521N, R521V, T522N, H524R, H524V, P525T, S526G, W527N, W527R, W527S, A528E, G531T, A537P, S540E, R546L, R546P, R546S, T547A, T547S, T548F, T548K, T548P, G549P, G549W, G550S, A551Q, S553N, S554N, D555S, K556W, L558P, T559Y, S564C, S564F, A565E, A565I, A565R, G567Q, G567V, A570K, A570M, A572S, S574W, W576Y, Y577L, V579G, R582L, D583W, V585I, A586K, T588D, T592C, T592L, T592S, F594L, D595Q, P600A, P600E, P600G, P600S, G607Q, S608V, G609N, G609R, A610M, V611K, W614A, N619L, S620G, V625M, V625Y, F630G, T634V, Y635R, Q639R, D644C, H650R, W655R, N656V, A661E, P669T, V670C, Y673S, A676P, K680I, T684V, G687P, S688L, R692H, I694L, I694W, G695C, G695L, G695W, K697R, S698E, T700C, K701A, K701G, K701M, A705W, T709M, Y710T, V712M, E714K, A716R, D717G, D717S, K718A, D719T, D719V, T721N, T721S, T721W, H723G, M726D, L728S, N731S, W734G, W734V, A735Q, A735S, I739K, S740D, A741I, D745F, L750S, P752A, P752C, P752Y, G754P, T756N, T756S, E757A, A760N, A769F, A769I, A769N, A769V, A769Y, K770H, K772A, K772C, K772P, A773C, A773H, A773V, D774A, A775L, A775W, D776I, D776L, D776S, R777D, R777E, R777G, R777H, R777P, R777S, T781A, T781G, T781P, T781Y, A782Q, D783C, D783E, D783R, G784L, G784S, K785S, K785W, D786V, L787P, L787T, Y789V, V792G, D793H, V794T, T795P, N798I, G799K, G799L, G799M, G799Q, H800A, H800L, P803S, A806Q, R808C, T810P, V813T, K814H, G815A, A816C, A816F, K818F, K818L, K818W, G821E, G821I, V822D, V822E, S826L, H830R, D831I, D831P, D831V, S832R, A835D, A835H, A835W, D836E, D836H, D836S, D836Y, R838G, K839D, A840G, K844G, Q850C, Q850G, E854C, I858D, I858Q, I858Y, K863F, K863N, D865A, D865G, L867W, S870R, V872C, I874G, S883L, T887F, T887G, V888A, Y892P, Y892R, Y893E, T901V, T901Y, G902R, G902S, G902W, N903D, K904M, K904V, P905R, P905Y, I906W, P908I, D910W, E912T, R914A, R914F, R914K, R914Y, Y915A, Y915C, Y915M, Y915V, S916G, D917C, D917F, D917S, G918H, T919Q, D921V, R922A, Q923V, W927G, Q934S, I935P, A936Q, K937I, K937P, A938C, S940T, S942K, S942V, V943A, V943H, V943R, A944P, T946P, T946V, V947G, A948C, Q950D, V956Q, E961D, E961F, E961S, I962C, I962N, L965C, L965G, L965M, L965P, L965Y, L966A, Y968G, Y968V, S969C, S969I, S969M, P973C, T976K, T976P, P977T, A978M, V979R, L980H, L980N, P981M, P985L, P985W, A986I, V987K, D990W, S995L, N997K, D1001L, W1002A, W1002D, W1002H, W1002N, W1002P, T1003Y, K1004H, A1006S, D1007P, V1018K, A1022H, V1024G, G1026R, K1030W, I1035D, V1048M, V1048Q, G1050S, N1051E, A1052K, L1053A, Q1057E, Q1057R, D1066W, L1068C, I1071W, K1072G, K1072Q, T1082F, G1083E, G1084V, G1085S, P1088E, S1089Q, W1091A, W1091V, T1092Q, N1093P, N1093Q, W1094D, W1094E, S1097W, A1099V, N1102E, N1102H, A1104R, T1107S, E1109D, E1109L, E1109W, A1111G, E1113P, Q1114S, Q1114V, Q1115A, Q1115K, L1116K, L1116W, G1117, G1117S, G1117T, G1117V, G1117W, I1119N, R1125V, R1125W, S1127Q, N1128A, A1129E, V1130A, V1130G, V1130R, P1133D, P1133R, G1136E, K1137R, K1137S, Q1141P, A1144P, A1144S, D1145E, D1145R, D1145T, T1150K, D1151R, D1151W, V1167R, A1175N, G1178M, A1179W, T1180A, T1180G, T1180I, T1180M, T1180Q, T1180Y, V1184E, V1184L, V1184P, V1186S, N1188W, C1199T, K1208W, T1209E, T1209K, A1210D, A1210G, A1210R, A1210T, A1210W, T1211G, T1211P, T1211R, F1214A, F1214K, F1214V, V1215D, T1216L, T1216Q, N1217A, T1218W, S1219A, S1219F, L1222F, L1222V, S1223V, S1224P, S1224W, L1225E, L1225K, T1226P, N1228F, G1229V, T1230K, T1230W, K1231F, S1233P, A1238E, A1238R, N1243T, T1244Q, A1246M, D1250I, D1250S, D1250W, A1259K, P1265W, N1269A, N1269K, I1274F, I1274R, T1275W, S1277W, E1278Q, D1279R, D1279W, H1280E, V1281I, V1281W, K1284G, F1286E, T1287C, T1287W, I1288A, I1288D, I1288G, I1288K, E1302R, E1302S or D1304V.

101. The lactase variant of the preceding embodiment, wherein the variant has a decreased or an increased galactose inhibition, preferably measured as a galactose inhibition improvement factor (GI-IF) of at least 1.4 or at most 0.6, compared to the lactase of SEQ ID NO: 1.

102. The lactase variant of embodiment 1 which comprises one or more of the following substitutions, wherein each position corresponds to a position in SEQ ID NO: 1: E2V, D3I, D3V, S7P, M13R, S15C, S15I, S15K, T16C, V20P, Y21C, Y21R, Y21T, A24F, A24T, D26C, S27A, K28C, Q29L, Q29M, Q29R, R31E, R31V, T32S, K40F, K40I, D45P, V47K, V47R, A49D, A49H, A49T, D51I, D51K, D55H, D55M, S57C, S57E, S57G, A58T, W59P, V62N, L64E, K73D, S75G, S75L, S75R, Q76G, S77E, S77H, S77K, N78C, E79H, E79S, E79T, L85F, L85S, L85V, L85W, P86Q, G87D, G88F, G90T, W91Y, Y92H, Y92M, Y92S, Y92V, R93A, R93D, R93F, R93H, R93L, R93N, R93T, R93V, R93Y, K94S, S95C, S95G, S95I, F96A, F96C, F96I, F96V, T97F, I98H, R100T, D101A, D101P, L102P, I109M, N110T, F111A, F111L, V114R, Y115E, V120A, W121R, W121V, H130A, H130T, Y132E, F137L, G149M, E150L, V157P, E158H, N159T, L161K, L161S, L161W, P162N, P162T, P162W, R165H, W166S, Y167A, S170Q, I172P, R174E, V176E, V176K, T177K, L178I, L178Q, T179C, V180G, H185G, H185L, N188V, S197C, D217L, D217M, T218G, A220G, A222R, N223S, Q228R, K236M, T237D, I251Y, A252W, A252Y, P268R, S272W, V291P, L292V, D293C, D296F, E298I, E298R, F301C, W303A, T304P, G305N, G305P, T309D, F312H, K320V, K320W, S325G, M326T, H328C, H328M, H328T, A337G, A337L, R340A, A341M, E343N, Q345S, I348D, Q350G, T359E, T360V, N362S, N362T, P363I, P363L, P363M, A364I, A364M, A364P, A365C, L368Q, L368S, I369G, I369K, I369W, D370L, N373G, N373L, G376A, G376S, V377M, V379A, V380P, E381A, E381T, M386G, M386N, M386S, N388L, R389A, R389E, R389Q, R389S, R389T, S390C, S390D, S390P, S390T, G393R, G393V, T395C, E396K, E396L, E396V, E396W, G399S, K400A, K400C, K400N, K400V, W401F, W401K, F402W, F402Y, G403A, G403K, G403V, Q404F, Q404L, Q404M, Q404P, Q404V, A405C, A405E, A405R, A405T, I406D, I406N, A411E, K417F, T427D, T427Q, T427S, T429D, N431M, D433P, D433W, R434N, R434V, N435L, A436L, V439C, V439K, W442E, W442G, S443C, L444V, M448C, M448L, M449D, M449T, M449W, M449Y, E450F, E450S, E450T, E450V, E450W, G451C, G451F, G451L, G451P, G451V, G451W, I452K, G454L, S455P, S455R, V456D, V456E, V456F, V456K, S457T, S457V, G458A, G458P, F459R, K465Y, L466P, A468D, A472G, A472Y, S475E, R477L, M479G, K485E, K487F, S493Q, N499T, G505D, G505S, V506C, V506E, V506R, V507R, G508C, G508E, T509K, T509M, N510A, N510F, N510I, Y511A, S512F, S512T, S512Y, D513P, D513R, A515E, N516C, Y517G, D518Y, K519C, K519I, I520G, I520H, I520S, R521V, H524R, H524V, S526G, W527N, W527R, W527S, A528E, A537P, S540E, G549P, G549W, L558P, T559Y, S564C, S564F, A565E, A565I, A565R, G567Q, G567V, R582L, D583W, T592L, P600A, P600E, P600G, P600S, G607Q, W614A, N619L, V625M, V625Y, T634V, Q639R, W655R, A661E, P669T, Y673S, A676P, K680I, G687P, S688L, R692H, I694L, I694W, G695C, G695L, G695W, K697R, S698E, K701A, K701G, K701M, A705W, Y710T, V712M, E714K, A716R, D717G, D717S, K718A, D719T, D719V, T721N, T721S, T721W, H723G, M726D, L728S, W734G, A735Q, A735S, I739K, S740D, A741I, L750S, P752C, P752Y, T756N, E757A, A769F, A769I, A769N, A769V, A769Y, K770H, K772A, K772C, K772P, A773C, D774A, R777H, R777S, T781A, T781G, T781P, T781Y, A782Q, K785S, K785W, D786V, Y789V, N798I, G799K, G799L, P803S, A806Q, T810P, K818F, K818L, K818W, G821I, V822D, V822E, D831P, S832R, A835D, A835H, D836E, D836S, D836Y, K839D, A840G, E854C, I858D, D865A, D865G, L867W, V872C, I874G, S883L, T887G, V888A, Y892P, Y893E, T901Y, G902R, G902S, G902W, N903D, K904M, K904V, P905R, P905Y, I906W, P908I, D910W, E912T, R914A, R914F, R914K, R914Y, Y915A, Y915C, Y915M, Y915V, S916G, D917C, D917F, D917S, G918H, T919Q, D921V, R922A, Q923V, Q934S, I935P, A936Q, K937I, K937P, A938C, S942K, S942V, V943A, T946P, V947G, V956Q, E961D, E961F, E961S, I962C, I962N, L965C, L965G, L965M, L965P, L965Y, L966A, Y968G, Y968V, S969C, S969I, S969M, P973C, T976K, T976P, P977T, A978M, L980H, L980N, P981M, P985L, P985W, A986I, V987K, D990W, S995L, N997K, D1001L, W1002A, W1002D, W1002H, W1002N, W1002P, T1003Y, A1006S, D1007P, V1018K, A1022H, V1024G, K1030W, I1035D, V1048M, V1048Q, N1051E, A1052K, L1053A, Q1057E, T1082F, G1083E, G1084V, G1085S, P1088E, S1089Q, N1093P, N1093Q, W1094D, W1094E, A1099V, N1102H, E1109D, E1109L, E1109W, A1111G, E1113P, Q1114S, Q1114V, Q1115A, Q1115K, L1116K, L1116W, G1117I, G1117S, G1117T, G1117V, G1117W, I1119N, R1125V, R1125W, S1127Q, N1128A, A1129E, V1130A, V1130R, P1133D, P1133R, K1137R, K1137S, A1144P, A1144S, D1145E, D1145R, D1145T, T1150K, D1151W, V1167R, G1178M, A1179W, T1180A, T1180G, T1180I, T1180M, T1180Q, T1180Y, V1184E, V1184L, V1184P, V1186S, N1188W, K1208W, T1209E, T1209K, A1210D, A1210G, A1210R, A1210T, A1210W, T1211G, T1211P, T1211R, F1214A, F1214K, F1214V, V1215D, T1216L, T1216Q, N1217A, T1218W, L1222F, L1222V, S1224P, S1224W, L1225E, L1225K, T1226P, G1229V, T1230K, T1230W, A1238E, A1238R, A1259K, P1265W, I1274F, I1274R, T1275W, S1277W, E1278Q, D1279R, D1279W, H1280E, V1281I, V1281W, K1284G, F1286E, T1287C, T1287W, I1288A, I1288D, I1288G, I1288K, E1302R, E1302S or D1304V.

103. The lactase variant of the preceding embodiment, wherein the variant has a decreased galactose inhibition, preferably measured as a galactose inhibition improvement factor (GI-IF) of at least 1.4, compared to the lactase of SEQ ID NO: 1.

104. The lactase variant of embodiment 1 which comprises one or more of the following substitutions, wherein each position corresponds to a position in SEQ ID NO: 1: E2V, D3I, D3V, S7P, M13R, S15C, S15I, S15K, T16C, V20P, Y21C, Y21R, Y21T, A24F, A24T, D26C, S27A, K28C, Q29L, Q29M, Q29R, R31E, R31V, T32S, K40F, K40I, D45P, V47K, V47R, A49D, A49H, A49T, D51I, D51K, D55H, D55M, S57C, S57E, S57G, A58T, W59P, V62N, L64E, K73D, S75G, S75L, S75R, Q76G, S77E, S77H, S77K, N78C, E79H, E79S, E79T, L85F, L85S, L85V, L85W, P86Q, G87D, G88F, G90T, W91Y, Y92H, Y92M, Y92S, Y92V, R93A, R93D, R93F, R93H, R93L, R93N, R93T, R93V, R93Y, K94S, S95C, S95G, S95I, F96A, F96C, F96I, F96V, T97F, I98H, R100T, D101A, D101P, L102P, I109M, N110T, F111A, F111L, V114R, Y115E, V120A, W121R, W121V, H130A, H130T, Y132E, F137L, G149M, E150L, V157P, E158H, N159T, L161K, L161S, L161W, P162N, P162T, P162W, R165H, W166S, Y167A, S170Q, I172P, R174E, V176E, V176K, T177K, L178I, L178Q, T179C, V180G, H185G, H185L, N188V, S197C, D217L, D217M, T218G, A220G, A222R, N223S, Q228R, K236M, T237D, I251Y, A252W, A252Y, P268R, S272W, V291P, L292V, D293C, D296F, E298I, E298R, F301C, W303A, T304P, G305N, G305P, T309D, F312H, K320V, K320W, S325G, M326T, H328C, H328M, H328T, A337G, A337L, R340A, A341M, E343N, Q345S, I348D, Q350G, T359E, T360V, N362S, N362T, P363I, P363L, P363M, A364I, A364M, A364P, A365C, L368Q, L368S, I369G, I369K, I369W, D370L, N373G, N373L, G376A, G376S, V377M, V379A, V380P, E381A, E381T, M386G, M386N, M386S, N388L, R389A, R389E, R389Q, R389S, R389T, S390C, S390D, S390P, S390T, G393R, G393V, T395C, E396K, E396L, E396V, E396W, G399S, K400A, K400C, K400N, K400V, W401F, W401K, F402W, F402Y, G403A, G403K, G403V, Q404F, Q404L, Q404M, Q404P, Q404V, A405C, A405E, A405R, I406D, I406N, A411E, K417F, T427D, T427Q, T427S, T429D, N431M, D433P, D433W, R434N, R434V, N435L, A436L, V439C, V439K, W442E, W442G, S443C, L444V, M448C, M448L, M449D, M449T, M449W, M449Y, E450F, E450S, E450T, E450V, E450W, G451C, G451F, G451L, G451P, G451V, G451W, I452K, G454L, S455P, S455R, V456D, V456E, V456F, V456K, S457T, S457V, G458A, G458P, F459R, K465Y, L466P, A468D, A472G, A472Y, S475E, R477L, M479G, K485E, K487F, S493Q, N499T, G505D, G505S, V506C, V506E, V506R, V507R, G508C, G508E, T509K, T509M, N510A, N510F, N510I, Y511A, S512F, S512T, S512Y, D513P, D513R, A515E, N516C, Y517G, D518Y, K519C, K519I, I520G, I520H, I520S, R521V, H524R, H524V, S526G, W527N, W527R, W527S, A528E, A537P, S540E, G549P, G549W, L558P, T559Y, S564C, S564F, A565E, A565I, A565R, G567Q, G567V, R582L, D583W, T592L, P600A, P600E, P600G, P600S, G607Q, W614A, N619L, V625M, V625Y, T634V, Q639R, W655R, A661E, P669T, Y673S, A676P, K680I, G687P, S688L, R692H, I694L, I694W, G695C, G695L, G695W, K697R, S698E, K701A, K701G, K701M, A705W, Y710T, V712M, E714K, A716R, D717G, D717S, K718A, D719T, D719V, T721N, T721S, T721W, H723G, M726D, L728S, W734G, A735Q, A735S, I739K, S740D, A741I, L750S, P752C, P752Y, T756N, E757A, A769F, A769I, A769N, A769V, A769Y, K770H, K772A, K772C, K772P, A773C, D774A, R777H, R777S, T781A, T781G, T781P, T781Y, A782Q, K785S, K785W, D786V, Y789V, N798I, G799K, G799L, P803S, A806Q, T810P, K818F, K818L, K818W, G821I, V822D, V822E, D831P, S832R, A835D, A835H, D836E, D836S, D836Y, K839D, A840G, E854C, I858D, D865A, D865G, L867W, V872C, I874G, S883L, T887G, V888A, Y892P, Y893E, T901Y, G902R, G902S, G902W, N903D, K904M, K904V, P905R, P905Y, I906W, P908I, D910W, E912T, R914A, R914F, R914K, R914Y, Y915A, Y915C, Y915M, Y915V, S916G, D917C, D917F, D917S, G918H, T919Q, D921V, R922A, Q923V, Q934S, I935P, A936Q, K937I, K937P, A938C, S942K, S942V, V943A, T946P, V947G, V956Q, E961D, E961F, E961S, I962C, I962N, L965C, L965G, L965M, L965P, L965Y, L966A, Y968G, Y968V, S969C, S969I, S969M, P973C, T976K, T976P, P977T, A978M, L980H, L980N, P981M, P985L, P985W, A986I, V987K, D990W, S995L, N997K, D1001L, W1002A, W1002D, W1002H, W1002N, W1002P, T1003Y, A1006S, D1007P, V1018K, A1022H, V1024G, K1030W, I1035D, V1048M, V1048Q, N1051E, A1052K, L1053A, Q1057E, T1082F, G1083E, G1084V, G1085S, P1088E, S1089Q, N1093P, N1093Q, W1094D, W1094E, A1099V, N1102H, E1109D, E1109L, E1109W, A1111G, E1113P, Q1114S, Q1114V, Q1115A, Q1115K, L1116K, L1116W, G1117I, G1117S, G1117T, G1117V, G1117W, I1119N, R1125V, R1125W, S1127Q, N1128A, A1129E, V1130A, V1130R, P1133D, P1133R, K1137R, K1137S, A1144P, A1144S, D1145E, D1145R, D1145T, T1150K, D1151W, V1167R, G1178M, A1179W, T1180A, T1180G, T1180I, T1180M, T1180Q, T1180Y, V1184E, V1184L, V1184P, V1186S, N1188W, K1208W, T1209E, T1209K, A1210D, A1210G, A1210R, A1210T, A1210W, T1211G, T1211P, T1211R, F1214A, F1214K, F1214V, V1215D, T1216L, T1216Q, N1217A, T1218W, L1222F, L1222V, S1224P, S1224W, L1225E, L1225K, T1226P, G1229V, T1230K, T1230W, A1238E, A1238R, A1259K, P1265W, I1274F, I1274R, T1275W, S1277W, E1278Q, D1279R, D1279W, H1280E, V1281I, V1281W, K1284G, F1286E, T1287C, T1287W, I1288A, I1288D, I1288G, I1288K, E1302R, E1302S or D1304V.

105. The lactase variant of the preceding embodiment, wherein the variant has a decreased galactose inhibition, preferably measured as a galactose inhibition improvement factor (GI-IF) of at least 1.4, compared to the lactase of SEQ ID NO: 1.

106. The lactase variant of embodiment 1 which comprises one or more of the following substitutions, wherein each position corresponds to a position in SEQ ID NO: 1: S7P, S15I, S15K, S15Y, T16C, V20P, A24T, S27A, K28C, Q29L, R31E, T32S, D45P, V47R, A49D, A49H, A49R, D51I, D55H, S57C, S57E, S57G, A58T, W59P, H66Y, S75G, Q76G, S77F, S77H, S77K, E81Q, L85F, L85W, P86Q, G87D, G88F, G90T, Y92M, Y92S, Y92V, R93A, R93F, R93H, R93L, R93N, R93T, R93V, R93Y, K94S, S95G, S95I, F96A, F96C, F96S, F96V, I98H, D101P, L102P, I109M, D112T, V120A, W121R, W121V, G124E, F137L, G149M, I153A, E158H, N159T, L161S, L161W, P162N, P162T, R165H, W166S, Y167A, S170Q, I172P, R174E, L178I, L178Q, H185L, N188V, N189E, D217L, D217M, T218G, A220G, A222R, N223S, K236M, T237D, A239G, A239T, A247R, I251Y, A252Y, G254I, T264M, P268R, S272W, Y279W, V291P, E298I, F301C, W303A, T304P, G305W, M326T, A337L, A341M, Q345S, I348D, T359E, N362T, P363I, A364I, A364M, L368S, I369K, I369W, D370L, N373G, G376S, E381A, E381T, M386G, M386N, M386S, W387L, R389E, R389Q, R389S, R389T, S390C, S390D, S390P, S390Q, S390T, K391E, N392D, G393R, G393V, N394L, T395C, T395H, T395N, T395S, T395W, E396K, E396L, E396V, E396W, Y398N, G399S, K400A, K400C, K400N, K400P, K400S, K400V, W401F, W401K, F402W, F402Y, G403A, G403K, G403V, Q404F, Q404H, Q404L, Q404M, Q404P, Q404V, A405C, A405E, A405R, A405T, I406D, I406N, L413P, G414A, D416I, K417F, D418R, T420K, T420R, K423D, F424L, T427Q, T427S, T429D, N431M, D433W, R434N, R434V, N435L, A436L, V439C, V439G, V439K, W442G, L444V, G445C, G445V, M448L, M449D, M449T, M449W, M449Y, E450F, E450S, E450T, E450V, E450W, G451C, G451F, G451L, G451P, G451V, G451W, I452K, S455P, S455R, V456D, V456F, S457V, G458A, F459R, K465Y, L466P, A468D, A472G, A472Y, M479G, M479R, K487F, A488N, N491W, S493M, N499T, N499Y, L500A, G504H, G505D, G505S, V506C, V506E, V506R, V507L, V507R, G508C, G508E, T509K, T509M, N510A, N510F, N510I, Y511A, S512F, S512T, D513L, D513P, D513R, A515E, N516C, Y517G, D518Y, R521N, R521V, T522N, H524R, H524V, S526G, W527N, W527R, A537P, S540E, R546L, R546S, T548P, G549W, G550S, A551Q, S554N, D555S, K556W, L558P, S564C, S564F, A565I, A565R, G567Q, G567V, A570K, S574W, R582L, D583W, A586K, T592C, T592S, F594L, D595Q, P600A, P600E, P600S, S608V, G609N, G609R, W614A, V625M, V625Y, F630G, Q639R, D644C, H650R, W655R, N656V, A661E, P669T, V670C, Y673S, S688L, I694L, I694W, G695C, G695L, K697R, S698E, T700C, K701A, K701G, K701M, T709M, Y710T, E714K, D717G, K718A, D719T, D719V, T721S, L728S, N731S, W734V, A735S, I739K, A741I, D745F, L750S, P752C, P752Y, G754P, T756N, E757A, A769F, A769I, A769N, A769V, K770H, K772A, K772P, A775L, A775W, D776I, D776L, D776S, R777D, R777G, R777H, R777P, T781A, T781G, T781P, T781Y, D783C, D783E, D783R, G784L, K785S, K785W, D786V, L787P, V792G, D793H, V794T, T795P, G799K, G799L, G799M, P803S, T810P, K814H, G815A, A816C, A816F, K818F, K818W, G821I, V822D, S826L, H830R, A835D, A835H, D836E, D836H, D836S, D836Y, K839D, K844G, Q850G, E854C, D865G, L867W, V872C, S883L, Y893E, T901V, T901Y, G902S, K904V, E912T, R914A, R914F, R914K, R914Y, Y915C, Y915M, Y915V, S916G, D917F, D917S, G918H, T919Q, D921V, R922A, Q923V, Q934S, I935P, A936Q, K937I, K937P, V943A, V943H, V943R, A944P, T946V, V956Q, E961D, E961F, E961S, L965C, L965G, L965M, L965P, L965Y, Y968G, S969I, S969M, P973C, P981M, P985L, A986I, W1002A, W1002D, W1002N, W1002P, A1006S, D1007P, K1030W, I1035D, V1048Q, G1050S, N1051E, L1053A, D1066W, L1068C, G1084V, G1085S, S1089Q, N1093P, W1094D, W1094E, S1097W, N1102H, E1109D, E1109L, E1113P, Q1114S, Q1114V, Q1115A, L1116K, L1116W, G1117S, G1117T, G1117V, G1117W, I1119N, R1125V, R1125W, S1127Q, N1128A, A1129E, V1130R, P1133R, K1137S, A1144S, D1145E, D1145R, D1145T, T1150K, D1151R, D1151W, V1167R, G1178M, A1179W, T1180G, T1180I, T1180M, T1180Q, V1184E, V1184L, V1184P, K1208W, A1210D, A1210G, A1210R, A1210T, T1211G, T1211P, F1214A, F1214K, F1214V, T1216L, T1216Q, S1219A, L1222V, S1223V, S1224P, L1225E, L1225K, T1226P, G1229V, T1230W, A1238E, A1259K, P1265W, N1269A, I1274F, I1274R, T1275W, S1277W, E1278Q, D1279R, D1279W, V1281I, V1281W, K1284G, F1286E, T1287C, T1287W, I1288A, I1288D, I1288G, I1288K, E1302R, E1302S or D1304V.

107. The lactase variant of the preceding embodiment, wherein the variant has a decreased or an increased galactose inhibition, preferably measured as a galactose inhibition improvement factor (GI-IF) of at least 1.5 or at most 0.5, compared to the lactase of SEQ ID NO: 1.

108. The lactase variant of embodiment 1 which comprises one or more of the following substitutions, wherein each position corresponds to a position in SEQ ID NO: 1: S7P, S15I, S15K, S15Y, T16C, V20P, A24T, S27A, K28C, Q29L, R31E, T32S, D45P, V47R, A49D, A49H, A49R, D51I, D55H, S57C, S57E, S57G, A58T, W59P, H66Y, S75G, Q76G, S77F, S77H, S77K, E81Q, L85F, L85W, P86Q, G87D, G88F, G90T, Y92M, Y92S, Y92V, R93A, R93F, R93H, R93L, R93N, R93T, R93V, R93Y, K94S, S95G, S95I, F96A, F96C, F96S, F96V, I98H, D101P, L102P, I109M, D112T, V120A, W121R, W121V, G124E, F137L, G149M, I153A, E158H, N159T, L161S, L161W, P162N, P162T, R165H, W166S, Y167A, S170Q, I172P, R174E, L178I, L178Q, H185L, N188V, N189E, D217L, D217M, T218G, A220G, A222R, N223S, K236M, T237D, A239G, A239T, A247R, I251Y, A252Y, G254I, T264M, P268R, S272W, Y279W, V291P, E298I, F301C, W303A, T304P, G305W, M326T, A337L, A341M, Q345S, I348D, T359E, N362T, P363I, A364I, A364M, L368S, I369K, I369W, D370L, N373G, G376S, E381A, E381T, M386G, M386N, M386S, W387L, R389E, R389Q, R389S, R389T, S390C, S390D, S390P, S390Q, S390T, K391E, N392D, G393R, G393V, N394L, T395C, T395H, T395N, T395S, T395W, E396K, E396L, E396V, E396W, Y398N, G399S, K400A, K400C, K400N, K400P, K400S, K400V, W401F, W401K, F402W, F402Y, G403A, G403K, G403V, Q404F, Q404H, Q404L, Q404M, Q404P, Q404V, A405C, A405E, A405R, I406D, I406N, L413P, G414A, D416I, K417F, D418R, T420K, T420R, K423D, F424L, T427Q, T427S, T429D, N431M, D433W, R434N, R434V, N435L, A436L, V439C, V439G, V439K, W442G, L444V, G445C, G445V, M448L, M449D, M449T, M449W, M449Y, E450F, E450S, E450T, E450V, E450W, G451C, G451F, G451L, G451P, G451V, G451W, I452K, S455P, S455R, V456D, V456F, S457V, G458A, F459R, K465Y, L466P, A468D, A472G, A472Y, M479G, M479R, K487F, A488N, N491W, S493M, N499T, N499Y, L500A, G504H, G505D, G505S, V506C, V506E, V506R, V507L, V507R, G508C, G508E, T509K, T509M, N510A, N510F, N510I, Y511A, S512F, S512T, D513L, D513P, D513R, A515E, N516C, Y517G, D518Y, R521N, R521V, T522N, H524R, H524V, S526G, W527N, W527R, A537P, S540E, R546L, R546S, T548P, G549W, G550S, A551Q, S554N, D555S, K556W, L558P, S564C, S564F, A565I, A565R, G567Q, G567V, A570K, S574W, R582L, D583W, A586K, T592C, T592S, F594L, D595Q, P600A, P600E, P600S, S608V, G609N, G609R, W614A, V625M, V625Y, F630G, Q639R, D644C, H650R, W655R, N656V, A661E, P669T, V670C, Y673S, S688L, I694L, I694W, G695C, G695L, K697R, S698E, T700C, K701A, K701G, K701M, T709M, Y710T, E714K, D717G, K718A, D719T, D719V, T721S, L728S, N731S, W734V, A735S, I739K, A741I, D745F, L750S, P752C, P752Y, G754P, T756N, E757A, A769F, A769I, A769N, A769V, K770H, K772A, K772P, A775L, A775W, D776I, D776L, D776S, R777D, R777G, R777H, R777P, T781A, T781G, T781P, T781Y, D783C, D783E, D783R, G784L, K785S, K785W, D786V, L787P, V792G, D793H, V794T, T795P, G799K, G799L, G799M, P803S, T810P, K814H, G815A, A816C, A816F, K818F, K818W, G821I, V822D, S826L, H830R, A835D, A835H, D836E, D836H, D836S, D836Y, K839D, K844G, Q850G, E854C, D865G, L867W, V872C, S883L, Y893E, T901V, T901Y, G902S, K904V, E912T, R914A, R914F, R914K, R914Y, Y915C, Y915M, Y915V, S916G, D917F, D917S, G918H, T919Q, D921V, R922A, Q923V, Q934S, I935P, A936Q, K937I, K937P, V943A, V943H, V943R, A944P, T946V, V956Q, E961D, E961F, E961S, L965C, L965G, L965M, L965P, L965Y, Y968G, S969I, S969M, P973C, P981M, P985L, A986I, W1002A, W1002D, W1002N, W1002P, A1006S, D1007P, K1030W, I1035D, V1048Q, G1050S, N1051E, L1053A, D1066W, L1068C, G1084V, G1085S, S1089Q, N1093P, W1094D, W1094E, S1097W, N1102H, E1109D, E1109L, E1113P, Q1114S, Q1114V, Q1115A, L1116K, L1116W, G1117S, G1117T, G1117V, G1117W, I1119N, R1125V, R1125W, S1127Q, N1128A, A1129E, V1130R, P1133R, K1137S, A1144S, D1145E, D1145R, D1145T, T1150K, D1151R, D1151W, V1167R, G1178M, A1179W, T1180G, T1180I, T1180M, T1180Q, V1184E, V1184L, V1184P, K1208W, A1210D, A1210G, A1210R, A1210T, T1211G, T1211P, F1214A, F1214K, F1214V, T1216L, T1216Q, S1219A, L1222V, S1223V, S1224P, L1225E, L1225K, T1226P, G1229V, T1230W, A1238E, A1259K, P1265W, N1269A, I1274F, I1274R, T1275W, S1277W, E1278Q, D1279R, D1279W, V1281I, V1281W, K1284G, F1286E, T1287C, T1287W, I1288A, I1288D, I1288G, I1288K, E1302R, E1302S or D1304V.

109. The lactase variant of the preceding embodiment, wherein the variant has a decreased or an increased galactose inhibition, preferably measured as a galactose inhibition improvement factor (GI-IF) of at least 1.5 or at most 0.5, compared to the lactase of SEQ ID NO: 1.

110. The lactase variant of embodiment 1 which comprises one or more of the following substitutions, wherein each position corresponds to a position in SEQ ID NO: 1: S7P, S15I, S15K, T16C, V20P, A24T, S27A, K28C, Q29L, R31E, T32S, D45P, V47R, A49D, A49H, D51I, D55H, S57C, S57E, S57G, A58T, W59P, S75G, Q76G, S77H, S77K, L85F, L85W, P86Q, G87D, G88F, G90T, Y92M, Y92S, Y92V, R93A, R93F, R93H, R93L, R93N, R93T, R93V, R93Y, K94S, S95G, S95I, F96A, F96C, F96V, I98H, D101P, L102P, I109M, V120A, W121R, W121V, F137L, G149M, E158H, N159T, L161S, L161W, P162N, P162T, R165H, W166S, Y167A, S170Q, I172P, R174E, L178I, L178Q, H185L, N188V, D217L, D217M, T218G, A220G, A222R, N223S, K236M, T237D, I251Y, A252Y, P268R, S272W, V291P, E298I, F301C, W303A, T304P, M326T, A337L, A341M, Q345S, I348D, T359E, N362T, P363I, A364I, A364M, L368S, I369K, I369W, D370L, N373G, G376S, E381A, E381T, M386G, M386N, M386S, R389E, R389Q, R389S, R389T, S390C, S390D, S390P, S390T, G393R, G393V, T395C, E396K, E396L, E396V, E396W, G399S, K400A, K400C, K400N, K400V, W401F, W401K, F402W, F402Y, G403A, G403K, G403V, Q404F, Q404L, Q404M, Q404P, Q404V, A405C, A405E, A405R, A405T, I406D, I406N, K417F, T427Q, T427S, T429D, N431M, D433W, R434N, R434V, N435L, A436L, V439C, V439K, W442G, L444V, M448L, M449D, M449T, M449W, M449Y, E450F, E450S, E450T, E450V, E450W, G451C, G451F, G451L, G451P, G451V, G451W, I452K, S455P, S455R, V456D, V456F, S457V, G458A, F459R, K465Y, L466P, A468D, A472G, A472Y, M479G, K487F, N499T, G505D, G505S, V506C, V506E, V506R, V507R, G508C, G508E, T509K, T509M, N510A, N510F, N510I, Y511A, S512F, S512T, D513P, D513R, A515E, N516C, Y517G, D518Y, R521V, H524R, H524V, S526G, W527N, W527R, A537P, S540E, G549W, L558P, S564C, S564F, A565I, A565R, G567Q, G567V, R582L, D583W, P600A, P600E, P600S, W614A, V625M, V625Y, Q639R, W655R, A661E, P669T, Y673S, S688L, I694L, I694W, G695C, G695L, K697R, S698E, K701A, K701G, K701M, Y710T, E714K, D717G, K718A, D719T, D719V, T721S, L728S, A735S, I739K, A741I, L750S, P752C, P752Y, T756N, E757A, A769F, A769I, A769N, A769V, K770H, K772A, K772P, R777H, T781A, T781G, T781P, T781Y, K785S, K785W, D786V, G799K, G799L, P803S, T810P, K818F, K818W, G821I, V822D, A835D, A835H, D836E, D836S, D836Y, K839D, E854C, D865G, L867W, V872C, S883L, Y893E, T901Y, G902S, K904V, E912T, R914A, R914F, R914K, R914Y, Y915C, Y915M, Y915V, S916G, D917F, D917S, G918H, T919Q, D921V, R922A, Q923V, Q934S, I935P, A936Q, K937I, K937P, V943A, V956Q, E961D, E961F, E961S, L965C, L965G, L965M, L965P, L965Y, Y968G, S969I, S969M, P973C, P981M, P985L, A986I, W1002A, W1002D, W1002N, W1002P, A1006S, D1007P, K1030W, I1035D, V1048Q, N1051E, L1053A, G1084V, G1085S, S1089Q, N1093P, W1094D, W1094E, N1102H, E1109D, E1109L, E1113P, Q1114S, Q1114V, Q1115A, L1116K, L1116W, G1117S, G1117T, G1117V, G1117W, I1119N, R1125V, R1125W, S1127Q, N1128A, A1129E, V1130R, P1133R, K1137S, A1144S, D1145E, D1145R, D1145T, T1150K, D1151W, V1167R, G1178M, A1179W, T1180G, T1180I, T1180M, T1180Q, V1184E, V1184L, V1184P, K1208W, A1210D, A1210G, A1210R, A1210T, T1211G, T1211P, F1214A, F1214K, F1214V, T1216L, T1216Q, L1222V, S1224P, L1225E, L1225K, T1226P, G1229V, T1230W, A1238E, A1259K, P1265W, I1274F, I1274R, T1275W, S1277W, E1278Q, D1279R, D1279W, V1281I, V1281W, K1284G, F1286E, T1287C, T1287W, I1288A, I1288D, I1288G, I1288K, E1302R, E1302S or D1304V.

111. The lactase variant of the preceding embodiment, wherein the variant has a decreased galactose inhibition, preferably measured as a galactose inhibition improvement factor (GI-IF) of at least 1.5, compared to the lactase of SEQ ID NO: 1.

112. The lactase variant of embodiment 1 which comprises one or more of the following substitutions, wherein each position corresponds to a position in SEQ ID NO: 1: S7P, S15I, S15K, T16C, V20P, A24T, S27A, K28C, Q29L, R31E, T32S, D45P, V47R, A49D, A49H, D51I, D55H, S57C, S57E, S57G, A58T, W59P, S75G, Q76G, S77H, S77K, L85F, L85W, P86Q, G87D, G88F, G90T, Y92M, Y92S, Y92V, R93A, R93F, R93H, R93L, R93N, R93T, R93V, R93Y, K94S, S95G, S95I, F96A, F96C, F96V, I98H, D101P, L102P, I109M, V120A, W121R, W121V, F137L, G149M, E158H, N159T, L161S, L161W, P162N, P162T, R165H, W166S, Y167A, S170Q, I172P, R174E, L178I, L178Q, H185L, N188V, D217L, D217M, T218G, A220G, A222R, N223S, K236M, T237D, I251Y, A252Y, P268R, S272W, V291P, E298I, F301C, W303A, T304P, M326T, A337L, A341M, Q345S, I348D, T359E, N362T, P363I, A364I, A364M, L368S, I369K, I369W, D370L, N373G, G376S, E381A, E381T, M386G, M386N, M386S, R389E, R389Q, R389S, R389T, S390C, S390D, S390P, S390T, G393R, G393V, T395C, E396K, E396L, E396V, E396W, G399S, K400A, K400C, K400N, K400V, W401F, W401K, F402W, F402Y, G403A, G403K, G403V, Q404F, Q404L, Q404M, Q404P, Q404V, A405C, A405E, A405R, I406D, I406N, K417F, T427Q, T427S, T429D, N431M, D433W, R434N, R434V, N435L, A436L, V439C, V439K, W442G, L444V, M448L, M449D, M449T, M449W, M449Y, E450F, E450S, E450T, E450V, E450W, G451C, G451F, G451L, G451P, G451V, G451W, I452K, S455P, S455R, V456D, V456F, S457V, G458A, F459R, K465Y, L466P, A468D, A472G, A472Y, M479G, K487F, N499T, G505D, G505S, V506C, V506E, V506R, V507R, G508C, G508E, T509K, T509M, N510A, N510F, N510I, Y511A, S512F, S512T, D513P, D513R, A515E, N516C, Y517G, D518Y, R521V, H524R, H524V, S526G, W527N, W527R, A537P, S540E, G549W, L558P, S564C, S564F, A565I, A565R, G567Q, G567V, R582L, D583W, P600A, P600E, P600S, W614A, V625M, V625Y, Q639R, W655R, A661E, P669T, Y673S, S688L, I694L, I694W, G695C, G695L, K697R, S698E, K701A, K701G, K701M, Y710T, E714K, D717G, K718A, D719T, D719V, T721S, L728S, A735S, I739K, A741I, L750S, P752C, P752Y, T756N, E757A, A769F, A769I, A769N, A769V, K770H, K772A, K772P, R777H, T781A, T781G, T781P, T781Y, K785S, K785W, D786V, G799K, G799L, P803S, T810P, K818F, K818W, G821I, V822D, A835D, A835H, D836E, D836S, D836Y, K839D, E854C, D865G, L867W, V872C, S883L, Y893E, T901Y, G902S, K904V, E912T, R914A, R914F, R914K, R914Y, Y915C, Y915M, Y915V, S916G, D917F, D917S, G918H, T919Q, D921V, R922A, Q923V, Q934S, I935P, A936Q, K937I, K937P, V943A, V956Q, E961D, E961F, E961S, L965C, L965G, L965M, L965P, L965Y, Y968G, S969I, S969M, P973C, P981M, P985L, A986I, W1002A, W1002D, W1002N, W1002P, A1006S, D1007P, K1030W, I1035D, V1048Q, N1051E, L1053A, G1084V, G1085S, S1089Q, N1093P, W1094D, W1094E, N1102H, E1109D, E1109L, E1113P, Q1114S, Q1114V, Q1115A, L1116K, L1116W, G1117S, G1117T, G1117V, G1117W, I1119N, R1125V, R1125W, S1127Q, N1128A, A1129E, V1130R, P1133R, K1137S, A1144S, D1145E, D1145R, D1145T, T1150K, D1151W, V1167R, G1178M, A1179W, T1180G, T1180I, T1180M, T1180Q, V1184E, V1184L, V1184P, K1208W, A1210D, A1210G, A1210R, A1210T, T1211G, T1211P, F1214A, F1214K, F1214V, T1216L, T1216Q, L1222V, S1224P, L1225E, L1225K, T1226P, G1229V, T1230W, A1238E, A1259K, P1265W, I1274F, I1274R, T1275W, S1277W, E1278Q, D1279R, D1279W, V1281I, V1281W, K1284G, F1286E, T1287C, T1287W, I1288A, I1288D, I1288G, I1288K, E1302R, E1302S or D1304V.

113. The lactase variant of the preceding embodiment, wherein the variant has a decreased galactose inhibition, preferably measured as a galactose inhibition improvement factor (GI-IF) of at least 1.5, compared to the lactase of SEQ ID NO: 1.

114. The lactase variant of embodiment 1 which comprises one or more of the following substitutions, wherein each position corresponds to a position in SEQ ID NO: 1: S7P, S15I, V20P, A24T, S27A, R31E, D45P, V47R, A49H, A49R, D51I, S57C, S57E, S57G, W59P, S77H, L85F, P86Q, G87D, G88F, G90T, Y92M, Y92S, Y92V, R93A, R93F, R93L, R93T, R93V, R93Y, K94S, F96A, F96C, F96S, F96V, D101P, D112T, W121R, L161S, P162N, P162T, R165H, W166S, Y167A, S170Q, R174E, H185L, D217L, D217M, T218G, A220G, A222R, N223S, K236M, T237D, A239G, A252Y, P268R, E298I, F301C, W303A, T304P, A337L, N362T, A364I, L368S, I369K, N373G, G376S, E381T, M386G, M386S, R389E, R389Q, R389S, S390C, S390D, S390Q, S390T, K391E, N392D, G393R, G393V, N394L, T395H, T395N, T395W, E396K, E396L, E396V, E396W, Y398N, K400N, K400S, W401K, F402W, F402Y, G403A, G403K, G403V, Q404F, Q404L, Q404M, Q404P, Q404V, A405C, A405T, I406D, I406N, D418R, T420R, T427Q, T427S, T429D, D433W, R434N, R434V, L444V, G445V, M448L, M449D, M449W, M449Y, E450F, E450S, E450T, E450V, E450W, G451C, G451F, G451L, G451P, G451W, I452K, S455P, S455R, V456F, F459R, L466P, A468D, A472G, A472Y, M479G, A488N, N491W, N499Y, G504H, G505D, G505S, V506C, V506E, V506R, V507L, V507R, G508C, G508E, T509K, N510A, N510F, N510I, Y511A, S512T, D513P, D513R, A515E, Y517G, R521N, R521V, T522N, H524R, S526G, W527N, R546S, G549W, G550S, S554N, D555S, K556W, L558P, S564F, G567Q, G567V, A570K, R582L, D583W, T592S, F594L, P600E, P600S, S608V, G609R, W614A, V625M, V625Y, F630G, Q639R, D644C, H650R, W655R, A661E, P669T, V670C, Y673S, S688L, I694L, G695C, G695L, S698E, T700C, K701A, K701G, K701M, Y710T, E714K, T721S, N731S, W734V, D745F, L750S, P752C, T756N, E757A, A769F, A769I, A769N, A769V, K770H, K772P, A775L, A775W, D776I, D776L, R777D, R777G, R777H, R777P, T781A, T781G, D783C, D783E, D783R, G784L, K785S, K785W, L787P, V792G, V794T, P803S, T810P, K814H, V822D, S826L, H830R, A835D, A835H, D836S, D836Y, K839D, K844G, D865G, L867W, V872C, G902S, E912T, R914A, R914F, R914K, Y915M, S916G, D917F, D917S, T919Q, R922A, Q923V, K937I, V943A, V943H, V943R, A944P, T946V, E961F, S969I, S969M, P973C, P985L, A986I, W1002A, W1002D, W1002N, W1002P, A1006S, D1007P, V1048Q, N1051E, D1066W, L1068C, G1084V, G1085S, N1093P, W1094D, S1097W, N1102H, E1109D, E1109L, E1113P, Q1114S, Q1114V, L1116K, G1117V, G1117W, R1125V, R1125W, S1127Q, A1129E, P1133R, K1137S, D1145E, D1145T, D1151R, V1167R, G1178M, A1179W, T1180Q, V1184E, V1184L, V1184P, A1210G, A1210T, T1211G, A1238E, P1265W, I1274F, I1274R, T1275W, S1277W, E1278Q, D1279R, D1279W, V1281I, K1284G, F1286E, T1287C, T1287W, I1288A, I1288D, I1288K, E1302R or E1302S.

115. The lactase variant of the preceding embodiment, wherein the variant has a decreased or an increased galactose inhibition, preferably measured as a galactose inhibition improvement factor (GI-IF) of at least 1.6 or at most 0.4, compared to the lactase of SEQ ID NO: 1.

116. The lactase variant of embodiment 1 which comprises one or more of the following substitutions, wherein each position corresponds to a position in SEQ ID NO: 1: S7P, S15I, V20P, A24T, S27A, R31E, D45P, V47R, A49H, A49R, D51I, S57C, S57E, S57G, W59P, S77H, L85F, P86Q, G87D, G88F, G90T, Y92M, Y92S, Y92V, R93A, R93F, R93L, R93T, R93V, R93Y, K94S, F96A, F96C, F96S, F96V, D101P, D112T, W121R, L161S, P162N, P162T, R165H, W166S, Y167A, S170Q, R174E, H185L, D217L, D217M, T218G, A220G, A222R, N223S, K236M, T237D, A239G, A252Y, P268R, E298I, F301C, W303A, T304P, A337L, N362T, A364I, L368S, I369K, N373G, G376S, E381T, M386G, M386S, R389E, R389Q, R389S, S390C, S390D, S390Q, S390T, K391E, N392D, G393R, G393V, N394L, T395H, T395N, T395W, E396K, E396L, E396V, E396W, Y398N, K400N, K400S, W401K, F402W, F402Y, G403A, G403K, G403V, Q404F, Q404L, Q404M, Q404P, Q404V, A405C, I406D, I406N, D418R, T420R, T427Q, T427S, T429D, D433W, R434N, R434V, L444V, G445V, M448L, M449D, M449W, M449Y, E450F, E450S, E450T, E450V, E450W, G451C, G451F, G451L, G451P, G451W, I452K, S455P, S455R, V456F, F459R, L466P, A468D, A472G, A472Y, M479G, A488N, N491W, N499Y, G504H, G505D, G505S, V506C, V506E, V506R, V507L, V507R, G508C, G508E, T509K, N510A, N510F, N510I, Y511A, S512T, D513P, D513R, A515E, Y517G, R521N, R521V, T522N, H524R, S526G, W527N, R546S, G549W, G550S, S554N, D555S, K556W, L558P, S564F, G567Q, G567V, A570K, R582L, D583W, T592S, F594L, P600E, P600S, S608V, G609R, W614A, V625M, V625Y, F630G, Q639R, D644C, H650R, W655R, A661E, P669T, V670C, Y673S, S688L, I694L, G695C, G695L, S698E, T700C, K701A, K701G, K701M, Y710T, E714K, T721S, N731S, W734V, D745F, L750S, P752C, T756N, E757A, A769F, A769I, A769N, A769V, K770H, K772P, A775L, A775W, D776I, D776L, R777D, R777G, R777H, R777P, T781A, T781G, D783C, D783E, D783R, G784L, K785S, K785W, L787P, V792G, V794T, P803S, T810P, K814H, V822D, S826L, H830R, A835D, A835H, D836S, D836Y, K839D, K844G, D865G, L867W, V872C, G902S, E912T, R914A, R914F, R914K, Y915M, S916G, D917F, D917S, T919Q, R922A, Q923V, K937I, V943A, V943H, V943R, A944P, T946V, E961F, S969I, S969M, P973C, P985L, A986I, W1002A, W1002D, W1002N, W1002P, A1006S, D1007P, V1048Q, N1051E, D1066W, L1068C, G1084V, G1085S, N1093P, W1094D, S1097W, N1102H, E1109D, E1109L, E1113P, Q1114S, Q1114V, L1116K, G1117V, G1117W, R1125V, R1125W, S1127Q, A1129E, P1133R, K1137S, D1145E, D1145T, D1151R, V1167R, G1178M, A1179W, T1180Q, V1184E, V1184L, V1184P, A1210G, A1210T, T1211G, A1238E, P1265W, I1274F, I1274R, T1275W, S1277W, E1278Q, D1279R, D1279W, V1281I, K1284G, F1286E, T1287C, T1287W, I1288A, I1288D, I1288K, E1302R or E1302S.

117. The lactase variant of the preceding embodiment, wherein the variant has a decreased or an increased galactose inhibition, preferably measured as a galactose inhibition improvement factor (GI-IF) of at least 1.6 or at most 0.4, compared to the lactase of SEQ ID NO: 1.

118. The lactase variant of embodiment 1 which comprises one or more of the following substitutions, wherein each position corresponds to a position in SEQ ID NO: 1: S7P, S15I, V20P, A24T, S27A, R31E, D45P, V47R, A49H, D51I, S57C, S57E, S57G, W59P, S77H, L85F, P86Q, G87D, G88F, G90T, Y92M, Y92S, Y92V, R93A, R93F, R93L, R93T, R93V, R93Y, K94S, F96A, F96C, F96V, D101P, W121R, L161S, P162N, P162T, R165H, W166S, Y167A, S170Q, R174E, H185L, D217L, D217M, T218G, A220G, A222R, N223S, K236M, T237D, A252Y, P268R, E2981, F301C, W303A, T304P, A337L, N362T, A364I, L368S, I369K, N373G, G376S, E381T, M386G, M386S, R389E, R389Q, R389S, S390C, S390D, S390T, G393R, G393V, E396K, E396L, E396V, E396W, K400N, W401K, F402W, F402Y, G403A, G403K, G403V, Q404F, Q404L, Q404M, Q404P, Q404V, A405C, A405T, I406D, I406N, T427Q, T427S, T429D, D433W, R434N, R434V, L444V, M448L, M449D, M449W, M449Y, E450F, E450S, E450T, E450V, E450W, G451C, G451F, G451L, G451P, G451W, I452K, S455P, S455R, V456F, F459R, L466P, A468D, A472G, A472Y, M479G, G505D, G505S, V506C, V506E, V506R, V507R, G508C, G508E, T509K, N510A, N510F, N510I, Y511A, S512T, D513P, D513R, A515E, Y517G, R521V, H524R, S526G, W527N, G549W, L558P, S564F, G567Q, G567V, R582L, D583W, P600E, P600S, W614A, V625M, V625Y, Q639R, W655R, A661E, P669T, Y673S, S688L, I694L, G695C, G695L, S698E, K701A, K701G, K701M, Y710T, E714K, T721S, L750S, P752C, T756N, E757A, A769F, A769I, A769N, A769V, K770H, K772P, R777H, T781A, T781G, K785S, K785W, P803S, T810P, V822D, A835D, A835H, D836S, D836Y, K839D, D865G, L867W, V872C, G902S, E912T, R914A, R914F, R914K, Y915M, S916G, D917F, D917S, T919Q, R922A, Q923V, K937I, V943A, E961F, S969I, S969M, P973C, P985L, A986I, W1002A, W1002D, W1002N, W1002P, A1006S, D1007P, V1048Q, N1051E, G1084V, G1085S, N1093P, W1094D, N1102H, E1109D, E1109L, E1113P, Q1114S, Q1114V, L1116K, G1117V, G1117W, R1125V, R1125W, S1127Q, A1129E, P1133R, K1137S, D1145E, D1145T, V1167R, G1178M, A1179W, T1180Q, V1184E, V1184L, V1184P, A1210G, A1210T, T1211G, T1211P, F1214K, F1214V, T1216Q, L1222V, L1225E, L1225K, T1226P, T1230W, A1238E, P1265W, I1274F, I1274R, T1275W, S1277W, E1278Q, D1279R, D1279W, V1281I, K1284G, F1286E, T1287C, T1287W, I1288A, I1288D, I1288K, E1302R or E1302S.

119. The lactase variant of the preceding embodiment, wherein the variant has a decreased galactose inhibition, preferably measured as a galactose inhibition improvement factor (GI-IF) of at least 1.6, compared to the lactase of SEQ ID NO: 1.

120. The lactase variant of embodiment 1 which comprises one or more of the following substitutions, wherein each position corresponds to a position in SEQ ID NO: 1: S7P, S15I, V20P, A24T, S27A, R31E, D45P, V47R, A49H, D51I, S57C, S57E, S57G, W59P, S77H, L85F, P86Q, G87D, G88F, G90T, Y92M, Y92S, Y92V, R93A, R93F, R93L, R93T, R93V, R93Y, K94S, F96A, F96C, F96V, D101P, W121R, L161S, P162N, P162T, R165H, W166S, Y167A, S170Q, R174E, H185L, D217L, D217M, T218G, A220G, A222R, N223S, K236M, T237D, A252Y, P268R, E2981, F301C, W303A, T304P, A337L, N362T, A364I, L368S, I369K, N373G, G376S, E381T, M386G, M386S, R389E, R389Q, R389S, S390C, S390D, S390T, G393R, G393V, E396K, E396L, E396V, E396W, K400N, W401K, F402W, F402Y, G403A, G403K, G403V, Q404F, Q404L, Q404M, Q404P, Q404V, A405C, I406D, I406N, T427Q, T427S, T429D, D433W, R434N, R434V, L444V, M448L, M449D, M449W, M449Y, E450F, E450S, E450T, E450V, E450W, G451C, G451F, G451L, G451P, G451W, I452K, S455P, S455R, V456F, F459R, L466P, A468D, A472G, A472Y, M479G, G505D, G505S, V506C, V506E, V506R, V507R, G508C, G508E, T509K, N510A, N510F, N510I, Y511A, S512T, D513P, D513R, A515E, Y517G, R521V, H524R, S526G, W527N, G549W, L558P, S564F, G567Q, G567V, R582L, D583W, P600E, P600S, W614A, V625M, V625Y, Q639R, W655R, A661E, P669T, Y673S, S688L, I694L, G695C, G695L, S698E, K701A, K701G, K701M, Y710T, E714K, T721S, L750S, P752C, T756N, E757A, A769F, A769I, A769N, A769V, K770H, K772P, R777H, T781A, T781G, K785S, K785W, P803S, T810P, V822D, A835D, A835H, D836S, D836Y, K839D, D865G, L867W, V872C, G902S, E912T, R914A, R914F, R914K, Y915M, S916G, D917F, D917S, T919Q, R922A, Q923V, K937I, V943A, E961F, S969I, S969M, P973C, P985L, A986I, W1002A, W1002D, W1002N, W1002P, A1006S, D1007P, V1048Q, N1051E, G1084V, G1085S, N1093P, W1094D, N1102H, E1109D, E1109L, E1113P, Q1114S, Q1114V, L1116K, G1117V, G1117W, R1125V, R1125W, S1127Q, A1129E, P1133R, K1137S, D1145E, D1145T, V1167R, G1178M, A1179W, T1180Q, V1184E, V1184L, V1184P, A1210G, A1210T, T1211G, T1211P, F1214K, F1214V, T1216Q, L1222V, L1225E, L1225K, T1226P, T1230W, A1238E, P1265W, I1274F, I1274R, T1275W, S1277W, E1278Q, D1279R, D1279W, V1281I, K1284G, F1286E, T1287C, T1287W, I1288A, I1288D, I1288K, E1302R or E1302S.

121. The lactase variant of the preceding embodiment, wherein the variant has a decreased galactose inhibition, preferably measured as a galactose inhibition improvement factor (GI-IF) of at least 1.6, compared to the lactase of SEQ ID NO: 1.

122. The lactase variant of embodiment 1 which comprises one or more of the following substitutions, wherein each position corresponds to a position in SEQ ID NO: 1: S7P, S15I, V20P, A24T, S27A, R31E, A49H, S57E, S57G, W59P, S77H, L85F, P86Q, G87D, G88F, Y92S, Y92V, R93A, R93F, R93L, R93T, R93V, R93Y, F96A, F96S, D101P, D112T, L161S, P162N, R165H, W166S, Y167A, S170Q, R174E, H185L, D217L, D217M, A220G, A222R, T237D, A239G, A252Y, P268R, F301C, T304P, A337L, N362T, A364I, L368S, I369K, G376S, E381T, M386S, R389Q, R389S, S390C, S390Q, N392D, G393R, G393V, N394L, T395N, T395W, E396K, E396L, E396V, E396W, W401K, F402W, F402Y, G403A, G403K, Q404L, Q404M, Q404P, Q404V, A405C, A405T, D418R, T427Q, T429D, R434N, R434V, L444V, M448L, M449D, M449W, M449Y, E450F, E450S, E450T, E450V, E450W, G451C, G451F, G451L, G451P, G451W, I452K, F459R, L466P, A468D, A472G, A472Y, M479G, N491W, N499Y, G504H, G505D, G505S, V506C, V506E, V506R, G508C, G508E, T509K, N510A, N510I, Y511A, D513P, A515E, Y517G, R521V, T522N, H524R, S526G, W527N, R546S, G549W, G550S, D555S, L558P, S564F, G567Q, G567V, R582L, P600E, P600S, S608V, G609R, W614A, V625M, V625Y, Q639R, H650R, A661E, V670C, Y673S, S688L, G695C, G695L, S698E, K701A, K701M, Y710T, E714K, T721S, W734V, D745F, L750S, T756N, E757A, A769F, A769N, A769V, K770H, K772P, A775W, D776I, D776L, T781A, D783C, D783E, D783R, G784L, K785W, T810P, K814H, V822D, A835D, A835H, D836S, D836Y, K844G, D865G, L867W, V872C, G902S, E912T, R914A, R914F, R914K, Y915M, S916G, D917F, D917S, T919Q, R922A, Q923V, V943R, A944P, E961F, S969M, P973C, P985L, A986I, W1002D, W1002N, A1006S, N1051E, G1084V, G1085S, N1093P, E1109D, E1109L, Q1114V, L1116K, G1117V, G1117W, R1125V, P1133R, G1178M, A1179W, V1184E, V1184L, A1210G, A1210T, T1211G, T1211P, F1214K, T1216Q, L1222V, L1225K, T1226P, T1230W, A1238E, P1265W, I1274F, T1275W, S1277W, E1278Q, D1279W, V1281I, K1284G, T1287C, T1287W, I1288A, I1288D, E1302R or E1302S.

123. The lactase variant of the preceding embodiment, wherein the variant has a decreased or an increased galactose inhibition, preferably measured as a galactose inhibition improvement factor (GI-IF) of at least 1.7 or at most 0.3, compared to the lactase of SEQ ID NO: 1.

124. The lactase variant of embodiment 1 which comprises one or more of the following substitutions, wherein each position corresponds to a position in SEQ ID NO: 1: S7P, S15I, V20P, A24T, S27A, R31E, A49H, S57E, S57G, W59P, S77H, L85F, P86Q, G87D, G88F, Y92S, Y92V, R93A, R93F, R93L, R93T, R93V, R93Y, F96A, F96S, D101P, D112T, L161S, P162N, R165H, W166S, Y167A, S170Q, R174E, H185L, D217L, D217M, A220G, A222R, T237D, A239G, A252Y, P268R, F301C, T304P, A337L, N362T, A364I, L368S, I369K, G376S, E381T, M386S, R389Q, R389S, S390C, S390Q, N392D, G393R, G393V, N394L, T395N, T395W, E396K, E396L, E396V, E396W, W401K, F402W, F402Y, G403A, G403K, Q404L, Q404M, Q404P, Q404V, A405C, D418R, T427Q, T429D, R434N, R434V, L444V, M448L, M449D, M449W, M449Y, E450F, E450S, E450T, E450V, E450W, G451C, G451F, G451L, G451P, G451W, I452K, F459R, L466P, A468D, A472G, A472Y, M479G, N491W, N499Y, G504H, G505D, G505S, V506C, V506E, V506R, G508C, G508E, T509K, N510A, N510I, Y511A, D513P, A515E, Y517G, R521V, T522N, H524R, S526G, W527N, R546S, G549W, G550S, D555S, L558P, S564F, G567Q, G567V, R582L, P600E, P600S, S608V, G609R, W614A, V625M, V625Y, Q639R, H650R, A661E, V670C, Y673S, S688L, G695C, G695L, S698E, K701A, K701M, Y710T, E714K, T721S, W734V, D745F, L750S, T756N, E757A, A769F, A769N, A769V, K770H, K772P, A775W, D776I, D776L, T781A, D783C, D783E, D783R, G784L, K785W, T810P, K814H, V822D, A835D, A835H, D836S, D836Y, K844G, D865G, L867W, V872C, G902S, E912T, R914A, R914F, R914K, Y915M, S916G, D917F, D917S, T919Q, R922A, Q923V, V943R, A944P, E961F, S969M, P973C, P985L, A986I, W1002D, W1002N, A1006S, N1051E, G1084V, G1085S, N1093P, E1109D, E1109L, Q1114V, L1116K, G1117V, G1117W, R1125V, P1133R, G1178M, A1179W, V1184E, V1184L, A1210G, A1210T, T1211G, T1211P, F1214K, T1216Q, L1222V, L1225K, T1226P, T1230W, A1238E, P1265W, I1274F, T1275W, S1277W, E1278Q, D1279W, V1281I, K1284G, T1287C, T1287W, I1288A, I1288D, E1302R or E1302S.

125. The lactase variant of the preceding embodiment, wherein the variant has a decreased or an increased galactose inhibition, preferably measured as a galactose inhibition improvement factor (GI-IF) of at least 1.7 or at most 0.3, compared to the lactase of SEQ ID NO: 1.

126. The lactase variant of embodiment 1 which comprises one or more of the following substitutions, wherein each position corresponds to a position in SEQ ID NO: 1: S7P, S15I, V20P, A24T, S27A, R31E, A49H, S57E, S57G, W59P, S77H, L85F, P86Q, G87D, G88F, Y92S, Y92V, R93A, R93F, R93L, R93T, R93V, R93Y, F96A, D101P, L161S, P162N, R165H, W166S, Y167A, S170Q, R174E, H185L, D217L, D217M, A220G, A222R, T237D, A252Y, P268R, F301C, T304P, A337L, N362T, A364I, L368S, I369K, G376S, E381T, M386S, R389Q, R389S, S390C, G393R, G393V, E396K, E396L, E396V, E396W, W401K, F402W, F402Y, G403A, G403K, Q404L, Q404M, Q404P, Q404V, A405C, A405T, T427Q, T429D, R434N, R434V, L444V, M448L, M449D, M449W, M449Y, E450F, E450S, E450T, E450V, E450W, G451C, G451F, G451L, G451P, G451W, I452K, F459R, L466P, A468D, A472G, A472Y, M479G, G505D, G505S, V506C, V506E, V506R, G508C, G508E, T509K, N510A, N510I, Y511A, D513P, A515E, Y517G, R521V, H524R, S526G, W527N, G549W, L558P, S564F, G567Q, G567V, R582L, P600E, P600S, W614A, V625M, V625Y, Q639R, A661E, Y673S, S688L, G695C, G695L, S698E, K701A, K701M, Y710T, E714K, T721S, L750S, T756N, E757A, A769F, A769N, A769V, K770H, K772P, T781A, K785W, T810P, V822D, A835D, A835H, D836S, D836Y, D865G, L867W, V872C, G902S, E912T, R914A, R914F, R914K, Y915M, S916G, D917F, D917S, T919Q, R922A, Q923V, E961F, S969M, P973C, P985L, A986I, W1002D, W1002N, A1006S, N1051E, G1084V, G1085S, N1093P, E1109D, E1109L, Q1114V, L1116K, G1117V, G1117W, R1125V, P1133R, G1178M, A1179W, V1184E, V1184L, A1210G, A1210T, T1211G, T1211P, F1214K, T1216Q, L1222V, L1225K, T1226P, T1230W, A1238E, P1265W, I1274F, T1275W, S1277W, E1278Q, D1279W, V1281I, K1284G, T1287C, T1287W, I1288A, I1288D, E1302R or E1302S.

127. The lactase variant of the preceding embodiment, wherein the variant has a decreased galactose inhibition, preferably measured as a galactose inhibition improvement factor (GI-IF) of at least 1.7, compared to the lactase of SEQ ID NO: 1.

128. The lactase variant of embodiment 1 which comprises one or more of the following substitutions, wherein each position corresponds to a position in SEQ ID NO: 1: S7P, S15I, V20P, A24T, S27A, R31E, A49H, S57E, S57G, W59P, S77H, L85F, P86Q, G87D, G88F, Y92S, Y92V, R93A, R93F, R93L, R93T, R93V, R93Y, F96A, D101P, L161S, P162N, R165H, W166S, Y167A, S170Q, R174E, H185L, D217L, D217M, A220G, A222R, T237D, A252Y, P268R, F301C, T304P, A337L, N362T, A364I, L368S, I369K, G376S, E381T, M386S, R389Q, R389S, S390C, G393R, G393V, E396K, E396L, E396V, E396W, W401K, F402W, F402Y, G403A, G403K, Q404L, Q404M, Q404P, Q404V, A405C, T427Q, T429D, R434N, R434V, L444V, M448L, M449D, M449W, M449Y, E450F, E450S, E450T, E450V, E450W, G451C, G451F, G451L, G451P, G451W, I452K, F459R, L466P, A468D, A472G, A472Y, M479G, G505D, G505S, V506C, V506E, V506R, G508C, G508E, T509K, N510A, N510I, Y511A, D513P, A515E, Y517G, R521V, H524R, S526G, W527N, G549W, L558P, S564F, G567Q, G567V, R582L, P600E, P600S, W614A, V625M, V625Y, Q639R, A661E, Y673S, S688L, G695C, G695L, S698E, K701A, K701M, Y710T, E714K, T721S, L750S, T756N, E757A, A769F, A769N, A769V, K770H, K772P, T781A, K785W, T810P, V822D, A835D, A835H, D836S, D836Y, D865G, L867W, V872C, G902S, E912T, R914A, R914F, R914K, Y915M, S916G, D917F, D917S, T919Q, R922A, Q923V, E961F, S969M, P973C, P985L, A986I, W1002D, W1002N, A1006S, N1051E, G1084V, G1085S, N1093P, E1109D, E1109L, Q1114V, L1116K, G1117V, G1117W, R1125V, P1133R, G1178M, A1179W, V1184E, V1184L, A1210G, A1210T, T1211G, T1211P, F1214K, T1216Q, L1222V, L1225K, T1226P, T1230W, A1238E, P1265W, I1274F, T1275W, S1277W, E1278Q, D1279W, V1281I, K1284G, T1287C, T1287W, I1288A, I1288D, E1302R or E1302S.

129. The lactase variant of the preceding embodiment, wherein the variant has a decreased galactose inhibition, preferably measured as a galactose inhibition improvement factor (GI-IF) of at least 1.7, compared to the lactase of SEQ ID NO: 1.

130. The lactase variant of embodiment 1 which comprises one or more of the following substitutions, wherein each position corresponds to a position in SEQ ID NO: 1: S7P, S15I, V20P, A24T, S57E, W59P, L85F, P86Q, R93A, R93F, R93L, R93T, R93Y, P162N, R165H, W166S, Y167A, R174E, D217L, A222R, T237D, P268R, T304P, A337L, N362T, A364I, L368S, I369K, G376S, M386S, R389Q, R389S, S390C, S390Q, N392D, G393V, N394L, T395N, T395W, E396L, E396V, W401K, F402Y, G403A, Q404M, Q404P, Q404V, A405C, A405T, T427Q, T429D, R434N, M448L, M449D, M449W, M449Y, E450F, E450S, E450T, E450V, E450W, G451C, G451F, G451L, G451P, G451W, F459R, A468D, A472G, A472Y, M479G, N491W, N499Y, G505D, G505S, V506E, G508C, G508E, T509K, N510A, N510I, Y511A, D513P, A515E, Y517G, R521V, S526G, W527N, R546S, S564F, G567Q, R582L, P600E, P600S, S608V, W614A, V625M, V625Y, H650R, V670C, Y673S, S688L, G695C, G695L, K701A, K701M, Y710T, E714K, T721S, D745F, L750S, T756N, E757A, A769F, A769N, A769V, K770H, K772P, D776I, T781A, D783E, G784L, K785W, T810P, K814H, A835D, A835H, K844G, E912T, R914A, R914F, D917S, T919Q, R922A, Q923V, E961F, P985L, A986I, A1006S, N1051E, N1093P, L1116K, G1117V, G1117W, R1125V, G1178M, A1179W, V1184E, A1210T, T1211G, T1211P, L1225K, A1238E, P1265W, I1274F, T1275W, S1277W, K1284G, T1287C, I1288A, I1288D, E1302R or E1302S.

131. The lactase variant of the preceding embodiment, wherein the variant has a decreased or an increased galactose inhibition, preferably measured as a galactose inhibition improvement factor (GI-IF) of at least 1.8 or at most 0.2, compared to the lactase of SEQ ID NO: 1.

132. The lactase variant of embodiment 1 which comprises one or more of the following substitutions, wherein each position corresponds to a position in SEQ ID NO: 1: S7P, S15I, V20P, A24T, S57E, W59P, L85F, P86Q, R93A, R93F, R93L, R93T, R93Y, P162N, R165H, W166S, Y167A, R174E, D217L, A222R, T237D, P268R, T304P, A337L, N362T, A364I, L368S, I369K, G376S, M386S, R389Q, R389S, S390C, S390Q, N392D, G393V, N394L, T395N, T395W, E396L, E396V, W401K, F402Y, G403A, Q404M, Q404P, Q404V, A405C, T427Q, T429D, R434N, M448L, M449D, M449W, M449Y, E450F, E450S, E450T, E450V, E450W, G451C, G451F, G451L, G451P, G451W, F459R, A468D, A472G, A472Y, M479G, N491W, N499Y, G505D, G505S, V506E, G508C, G508E, T509K, N510A, N510I, Y511A, D513P, A515E, Y517G, R521V, S526G, W527N, R546S, S564F, G567Q, R582L, P600E, P600S, S608V, W614A, V625M, V625Y, H650R, V670C, Y673S, S688L, G695C, G695L, K701A, K701M, Y710T, E714K, T721S, D745F, L750S, T756N, E757A, A769F, A769N, A769V, K770H, K772P, D776I, T781A, D783E, G784L, K785W, T810P, K814H, A835D, A835H, K844G, E912T, R914A, R914F, D917S, T919Q, R922A, Q923V, E961F, P985L, A986I, A1006S, N1051E, N1093P, L1116K, G1117V, G1117W, R1125V, G1178M, A1179W, V1184E, A1210T, T1211G, T1211P, L1225K, A1238E, P1265W, I1274F, T1275W, S1277W, K1284G, T1287C, I1288A, I1288D, E1302R or E1302S.

133. The lactase variant of the preceding embodiment, wherein the variant has a decreased or an increased galactose inhibition, preferably measured as a galactose inhibition improvement factor (GI-IF) of at least 1.8 or at most 0.2, compared to the lactase of SEQ ID NO: 1.

134. The lactase variant of embodiment 1 which comprises one or more of the following substitutions, wherein each position corresponds to a position in SEQ ID NO: 1: S7P, S15I, V20P, A24T, S57E, W59P, L85F, P86Q, R93A, R93F, R93L, R93T, R93Y, P162N, R165H, W166S, Y167A, R174E, D217L, A222R, T237D, P268R, T304P, A337L, N362T, A364I, L368S, I369K, G376S, M386S, R389Q, R389S, S390C, G393V, E396L, E396V, W401K, F402Y, G403A, Q404M, Q404P, Q404V, A405C, A405T, T427Q, T429D, R434N, M448L, M449D, M449W, M449Y, E450F, E450S, E450T, E450V, E450W, G451C, G451F, G451L, G451P, G451W, F459R, A468D, A472G, A472Y, M479G, G505D, G505S, V506E, G508C, G508E, T509K, N510A, N510I, Y511A, D513P, A515E, Y517G, R521V, S526G, W527N, S564F, G567Q, R582L, P600E, P600S, W614A, V625M, V625Y, Y673S, S688L, G695C, G695L, K701A, K701M, Y710T, E714K, T721S, L750S, T756N, E757A, A769F, A769N, A769V, K770H, K772P, T781A, K785W, T810P, A835D, A835H, E912T, R914A, R914F, D917S, T919Q, R922A, Q923V, E961F, P985L, A986I, A1006S, N1051E, N1093P, L1116K, G1117V, G1117W, R1125V, G1178M, A1179W, V1184E, A1210T, T1211G, T1211P, L1225K, A1238E, P1265W, I1274F, T1275W, S1277W, K1284G, T1287C, I1288A, I1288D, E1302R or E1302S.

135. The lactase variant of the preceding embodiment, wherein the variant has a decreased galactose inhibition, preferably measured as a galactose inhibition improvement factor (GI-IF) of at least 1.8, compared to the lactase of SEQ ID NO: 1.

136. The lactase variant of embodiment 1 which comprises one or more of the following substitutions, wherein each position corresponds to a position in SEQ ID NO: 1: S7P, S15I, V20P, A24T, S57E, W59P, L85F, P86Q, R93A, R93F, R93L, R93T, R93Y, P162N, R165H, W166S, Y167A, R174E, D217L, A222R, T237D, P268R, T304P, A337L, N362T, A364I, L368S, I369K, G376S, M386S, R389Q, R389S, S390C, G393V, E396L, E396V, W401K, F402Y, G403A, Q404M, Q404P, Q404V, A405C, T427Q, T429D, R434N, M448L, M449D, M449W, M449Y, E450F, E450S, E450T, E450V, E450W, G451C, G451F, G451L, G451P, G451W, F459R, A468D, A472G, A472Y, M479G, G505D, G505S, V506E, G508C, G508E, T509K, N510A, N510I, Y511A, D513P, A515E, Y517G, R521V, S526G, W527N, S564F, G567Q, R582L, P600E, P600S, W614A, V625M, V625Y, Y673S, S688L, G695C, G695L, K701A, K701M, Y710T, E714K, T721S, L750S, T756N, E757A, A769F, A769N, A769V, K770H, K772P, T781A, K785W, T810P, A835D, A835H, E912T, R914A, R914F, D917S, T919Q, R922A, Q923V, E961F, P985L, A986I, A1006S, N1051E, N1093P, L1116K, G1117V, G1117W, R1125V, G1178M, A1179W, V1184E, A1210T, T1211G, T1211P, L1225K, A1238E, P1265W, I1274F, T1275W, S1277W, K1284G, T1287C, I1288A, I1288D, E1302R or E1302S.

137. The lactase variant of the preceding embodiment, wherein the variant has a decreased galactose inhibition, preferably measured as a galactose inhibition improvement factor (GI-IF) of at least 1.8, compared to the lactase of SEQ ID NO: 1.

138. The lactase variant of embodiment 1 which comprises one or more of the following substitutions, wherein each position corresponds to a position in SEQ ID NO: 1: S7P, S15I, A24T, S57E, L85F, P86Q, R93A, R93F, R93L, R93Y, P162N, Y167A, R174E, D217L, A222R, T237D, P268R, T304P, N362T, A364I, I369K, M386S, R389Q, R389S, N392D, G393V, T395W, E396L, E396V, W401K, G403A, Q404M, Q404P, Q404V, A405T, T427Q, M448L, M449Y, E450F, E450S, E450T, E450V, E450W, G451C, G451F, G451L, G451P, F459R, A468D, A472G, A472Y, M479G, N491W, G505D, G505S, V506E, G508C, G508E, N510A, N510I, Y511A, D513P, A515E, Y517G, R521V, S526G, W527N, G567Q, P600E, P600S, V625M, V625Y, V670C, Y673S, S688L, G695L, K701A, Y710T, E714K, T721S, L750S, E757A, A769V, K772P, G784L, K785W, T810P, E912T, T919Q, R922A, Q923V, E961F, P985L, A1006S, N1051E, N1093P, L1116K, G1117W, A1179W, A1210T, T1211P, A1238E, P1265W, I1274F, T1275W, S1277W, K1284G, T1287C, I1288D, E1302R or E1302S.

139. The lactase variant of the preceding embodiment, wherein the variant has a decreased or an increased galactose inhibition, preferably measured as a galactose inhibition improvement factor (GI-IF) of at least 1.9 or at most 0.1, compared to the lactase of SEQ ID NO: 1.

140. The lactase variant of embodiment 1 which comprises one or more of the following substitutions, wherein each position corresponds to a position in SEQ ID NO: 1: S7P, S15I, A24T, S57E, L85F, P86Q, R93A, R93F, R93L, R93Y, P162N, Y167A, R174E, D217L, A222R, T237D, P268R, T304P, N362T, A364I, I369K, M386S, R389Q, R389S, N392D, G393V, T395W, E396L, E396V, W401K, G403A, Q404M, Q404P, Q404V, T427Q, M448L, M449Y, E450F, E450S, E450T, E450V, E450W, G451C, G451F, G451L, G451P, F459R, A468D, A472G, A472Y, M479G, N491W, G505D, G505S, V506E, G508C, G508E, N510A, N510I, Y511A, D513P, A515E, Y517G, R521V, S526G, W527N, G567Q, P600E, P600S, V625M, V625Y, V670C, Y673S, S688L, G695L, K701A, Y710T, E714K, T721S, L750S, E757A, A769V, K772P, G784L, K785W, T810P, E912T, T919Q, R922A, Q923V, E961F, P985L, A1006S, N1051E, N1093P, L1116K, G1117W, A1179W, A1210T, T1211P, A1238E, P1265W, I1274F, T1275W, S1277W, K1284G, T1287C, I1288D, E1302R or E1302S.

141. The lactase variant of the preceding embodiment, wherein the variant has a decreased or an increased galactose inhibition, preferably measured as a galactose inhibition improvement factor (GI-IF) of at least 1.9 or at most 0.1, compared to the lactase of SEQ ID NO: 1.

142. The lactase variant of embodiment 1 which comprises one or more of the following substitutions, wherein each position corresponds to a position in SEQ ID NO: 1: S7P, S15I, A24T, S57E, L85F, P86Q, R93A, R93F, R93L, R93Y, P162N, Y167A, R174E, D217L, A222R, T237D, P268R, T304P, N362T, A364I, I369K, M386S, R389Q, R389S, G393V, E396L, E396V, W401K, G403A, Q404M, Q404P, Q404V, A405T, T427Q, M448L, M449Y, E450F, E450S, E450T, E450V, E450W, G451C, G451F, G451L, G451P, F459R, A468D, A472G, A472Y, M479G, G505D, G505S, V506E, G508C, G508E, N510A, N510I, Y511A, D513P, A515E, Y517G, R521V, S526G, W527N, G567Q, P600E, P600S, V625M, V625Y, Y673S, S688L, G695L, K701A, Y710T, E714K, T721S, L750S, E757A, A769V, K772P, K785W, T810P, E912T, T919Q, R922A, Q923V, E961F, P985L, A1006S, N1051E, N1093P, L1116K, G1117W, A1179W, A1210T, T1211P, A1238E, P1265W, I1274F, T1275W, S1277W, K1284G, T1287C, I1288D, E1302R or E1302S.

143. The lactase variant of the preceding embodiment, wherein the variant has a decreased galactose inhibition, preferably measured as a galactose inhibition improvement factor (GI-IF) of at least 1.9, compared to the lactase of SEQ ID NO: 1.

144. The lactase variant of embodiment 1 which comprises one or more of the following substitutions, wherein each position corresponds to a position in SEQ ID NO: 1: S7P, S15I, A24T, S57E, L85F, P86Q, R93A, R93F, R93L, R93Y, P162N, Y167A, R174E, D217L, A222R, T237D, P268R, T304P, N362T, A364I, I369K, M386S, R389Q, R389S, G393V, E396L, E396V, W401K, G403A, Q404M, Q404P, Q404V, T427Q, M448L, M449Y, E450F, E450S, E450T, E450V, E450W, G451C, G451F, G451L, G451P, F459R, A468D, A472G, A472Y, M479G, G505D, G505S, V506E, G508C, G508E, N510A, N510I, Y511A, D513P, A515E, Y517G, R521V, S526G, W527N, G567Q, P600E, P600S, V625M, V625Y, Y673S, S688L, G695L, K701A, Y710T, E714K, T721S, L750S, E757A, A769V, K772P, K785W, T810P, E912T, T919Q, R922A, Q923V, E961F, P985L, A1006S, N1051E, N1093P, L1116K, G1117W, A1179W, A1210T, T1211P, A1238E, P1265W, I1274F, T1275W, S1277W, K1284G, T1287C, I1288D, E1302R or E1302S.

145. The lactase variant of the preceding embodiment, wherein the variant has a decreased galactose inhibition, preferably measured as a galactose inhibition improvement factor (GI-IF) of at least 1.9, compared to the lactase of SEQ ID NO: 1.

146. The lactase variant of embodiment 1, which comprises one or more of the following substitutions, wherein each position corresponds to a position in SEQ ID NO: 1: E2D, E2G, E2Q, E2V, D3A, D3H, D3I, D3N, D3S, D3V, D3W, A4C, A4G, A4H, A4I, A4L, A4M, A4P, T5A, T5D, T5F, T5K, T5R, T5S, T5V, R6A, R6D, R6F, R6G, R6H, R6L, R6M, R6P, R6S, R6W, S7A, S7D, S7I, S7L, S7N, S7P, S7T, S7W, S9G, S9H, S9P, S9R, S9W, T10G, T10K, T10L, T10P, T10R, T10S, T10W, T11L, T11P, Q12L, Q12R, Q12V, Q12Y, M13C, M13D, M13E, M13F, M13H, M13K, M13R, M13W, S14A, S14G, S14H, S14L, S14T, S14V, S14Y, S15C, S15F, S15I, S15K, S15L, S15P, S15R, S15T, S15V, S15W, S15Y, T16A, T16C, T16I, T16N, T16S, P17A, P17C, P17D, P17E, P17I, P17L, P17N, P17R, P17S, P17T, Y21A, Y21C, Y21D, Y21F, Y21G, Y21H, Y21M, Y21P, Y21R, Y21T, S22A, S22E, S22F, S22G, S22L, S22M, S22N, S22R, S22T, S22W, S23A, S23C, S23D, S23L, S23M, S23R, A24F, A24G, A24L, A24R, A24T, A24W, V25D, V25E, V25F, V25G, V25H, V25K, V25L, V25M, V25Q, V25R, V25S, V25T, V25W, D26C, D26I, D26L, D26M, D26T, D26V, S27A, S27C, S27F, S27G, S27H, S27P, S27Y, K28C, K28G, K28I, K28L, K28R, K28S, K28V, K28W, Q29D, Q29F, Q29G, Q29L, Q29M, Q29R, Q29S, Q29V, Q29W, R31E, R31G, R31I, R31M, R31V, T32M, T32Q, T32R, T32S, S33C, S33E, S33H, S33K, S33N, S33Q, S33R, S33V, D34C, D34E, D34F, D34G, D34H, D34L, D34S, D34W, D34Y, F35A, F35C, F35E, F35G, F35K, F35N, F35T, F35V, D36H, D36Q, A37N, A37Q, N38C, N38G, N38S, W39G, W39S, K40C, K40D, K40F, K40G, K40I, K40M, K40N, K40P, K40W, F41A, F41C, F41G, F41I, F41Q, F41S, F41Y, M42E, M42N, M42T, L43A, L43C, L43G, L43I, L43S, L43T, L43V, S44C, S44M, S44N, S44Y, D45A, D45L, D45P, D45V, V47K, V47R, Q48S, A49C, A49D, A49H, A49R, A49S, A49T, A49V, D51G, D51I, D51K, D51M, D51P, D51V, F54M, F54S, D55C, D55F, D55G, D55H, D55M, D55N, D55P, D55S, D55V, S57A, S57C, S57E, S57G, A58D, A58G, A58I, A58M, A58N, A58Q, A58R, A58T, W59D, W59I, W59K, W59L, W59N, W59P, W59V, Q60A, Q60C, Q60E, Q60F, Q60G, Q60K, Q60L, Q60M, Q60R, Q60S, Q60V, Q60Y, Q61K, Q61P, Q61S, V62G, V62N, V62S, V62T, V62W, D63G, D63L, D63P, D63S, D63V, L64E, L64G, H66C, H66L, H66R, H66T, H66W, H66Y, D67E, Y68P, Y68V, I70A, I70H, I70K, I70P, I70R, T71C, T71E, T71G, T71H, T71K, T71L, T71N, T71P, T71Q, T71R, T71S, K73A, K73D, K73F, K73G, K73Q, K73V, Y74G, Y74K, Y74T, Y74W, S75G, S75L, S75Q, S75R, S75V, Q76G, Q76I, Q76K, Q76M, Q76P, Q76S, Q76V, Q76Y, S77C, S77D, S77E, S77F, S77G, S77H, S77I, S77K, S77L, S77M, S77R, S77T, S77V, S77W, S77Y, N78C, N78E, N78F, N78K, N78Q, N78R, N78S, N78T, E79H, E79Q, E79S, E79T, E79W, A80K, E81A, E81Q, A83E, A83T, L85A, L85C, L85D, L85F, L85M, L85N, L85S, L85V, L85W, P86E, P86G, P86H, P86N, P86Q, P86R, P86V, P86W, P86Y, G87A, G87D, G87E, G87N, G87Q, G88A, G88F, G88I, G88M, G88Q, G88S, T89C, T89G, T89H, T89K, T89L, T89M, T89N, T89P, T89W, T89Y, G90A, G90C, G90D, G90L, G90S, G90T, G90V, W91E, W91L, W91P, W91Q, W91R, W91S, W91Y, Y92F, Y92H, Y92I, Y92M, Y92N, Y92S, Y92T, Y92V, Y92W, R93A, R93D, R93F, R93H, R93I, R93L, R93N, R93T, R93V, R93Y, K94A, K94C, K94G, K94R, K94S, K94T, K94V, S95A, S95C, S95D, S95E, S95G, S95I, S95L, S95Q, S95R, F96A, F96C, F96I, F96K, F96L, F96M, F96P, F96S, F96V, F96W, T97F, T97S, T97V, I98C, I98H, I98S, I98W, R100T, D101A, D101P, D101V, L102A, L102G, L102M, L102P, L102S, G104C, K105D, K105Q, K105R, K105W, K105Y, R106K, R106P, R106V, R106W, I107A, I107F, I107G, I107Q, I107S, A108E, A108S, A108V, I109M, I109T, N110A, N110F, N110S, N110T, N110V, N110W, F111A, F111C, F111L, F111Q, F111V, D112A, D112F, D112G, D112T, G113A, G113S, V114F, V114G, V114M, V114R, Y115E, M116A, M116C, M116D, M116W, M116Y, N117K, N117R, N117T, N117W, A118K, A118P, A118Y, T119A, T119G, T119L, V120A, V120K, W121C, W121D, W121R, W121T, W121V, W121Y, F122A, F122M, F122S, F122Y, N123P, G124E, G124M, G124Q, G124R, V125D, V125E, V125I, K126E, K126V, G128A, G128D, T129E, T129V, H130A, H130C, H130Q, H130S, H130T, P131K, P131L, P131S, Y132C, Y132E, Y132S, G133E, S135E, S135P, S135V, P136R, P136Y, F137A, F137C, F137D, F137G, F137L, F137P, S138A, S138D, S138G, S138H, S138L, S138M, S138R, S138V, F139A, F139E, F139Q, F139W, D140G, D140L, D140V, L141G, L141T, T142E, T142S, T142V, K146A, G148H, G148K, G148T, E150A, E150C, E150G, E150L, E150N, E150R, N151L, I153A, I153Y, V154E, V154I, V154K, V154L, V154M, V154S, V155F, V157A, V157G, V157L, V157P, V157Q, V157S, E158G, E158H, E158K, E158Q, E158V, N159D, N159H, N159S, N159T, R160G, L161E, L161K, L161M, L161S, L161W, P162F, P162G, P162N, P162T, P162W, S164A, R165H, W166S, Y167A, Y167C, S168C, G169A, G169C, G169D, G169S, S170L, S170Q, G171C, G171F, G171T, I172G, I172K, I172P, I172Q, Y173A, Y173H, Y173M, Y173P, Y173S, Y173W, R174E, R174K, D175E, D175Y, V176E, V176K, V176T, T177A, T177C, T177E, T177K, T177L, L178I, L178Q, L178W, T179A, T179C, T179D, T179H, T179I, T179K, T179L, T179N, T179P, T179S, V180A, V180C, V180D, V180E, V180G, V180M, T181A, T181D, T181F, T181K, T181R, D182F, D182L, D182S, G183W, V184F, V184H, V184P, V184Q, V184R, V184S, V184W, H185G, H185L, H185R, V186A, V186E, V186G, V186N, G187A, G187D, G187H, N188E, N188R, N188S, N188V, N188W, N189A, N189E, G190C, G190F, G190H, G190Q, G190V, V191C, V191T, V191W, V191Y, I193N, I193Q, I193T, I193V, K194A, K194I, K194L, K194R, T195A, T195E, T195M, T195S, T195W, P196A, P196H, P1961, P196M, P196S, P196W, S197A, S197C, S197E, S197K, S197L, S197P, L198E, L198F, L198H, L198I, L198K, L198R, L198V, L198W, A199E, A199F, A199K, A199P, A199R, A199T, Q201C, Q201E, Q201I, Q201K, Q201M, Q201V, N202A, N202D, N202F, N202G, N202K, N202L, N202M, N202Q, N202R, N202S, N202T, N202W, G203C, G203K, G203M, G203Q, G203R, G203S, G203V, G203W, G203Y, G204A, G204C, G204D, G204K, G204R, G204S, G204Y, V206A, V206C, V206D, V206F, V206G, V206I, V206K, V206Q, V206R, V206S, V206T, T207A, T207C, T207G, T207I, T207K, T207L, T207M, T207N, T207Q, T207R, T207W, M208A, M208S, M208T, N209C, N209D, N209G, N209K, N209L, N209Q, N209R, N209V, L210A, L210C, L210F, L210G, L210H, L210I, L210Q, L210R, L210S, L210T, L210V, T211A, T211D, T211E, T211F, T211K, T211N, T211Q, T211R, T211S, T212A, T212C, T212E, T212F, T212G, T212H, T212K, T212L, T212M, T212S, T212W, K213A, K213C, K213D, K213F, K213I, K213L, K213M, K213N, K213Q, K213R, K213S, K213T, K213V, K213Y, V214A, V214C, V214T, V214W, A215D, A215E, A215F, A215I, A215K, A215L, A215Q, A215R, A215S, A215V, N216D, N216T, N216V, D217F, D217G, D217L, D217M, D217T, D217V, T218D, T218G, T218H, A220C, A220G, A220I, A220L, A220M, A220T, A220V, A220W, A221C, A221D, A221E, A221L, A221N, A221R, A221V, A221Y, A222D, A222I, A222L, A222P, A222R, A222W, A222Y, N223A, N223E, N223F, N223G, N223K, N223L, N223M, N223R, N223S, N223T, N223V, N223W, I224G, I224Q, T225A, T225G, T225L, L226C, L226M, L226Q, K227T, Q228N, Q228R, T229A, T229C, T229D, T229G, T229H, T229M, T229N, T229Q, T229R, T229V, V230F, V230L, V230M, V230Q, V230R, V230S, F231A, F231E, F231G, F231I, F231K, F231L, F231Q, F231S, F231V, F231W, F231Y, P232G, P232H, P232L, P232M, P232R, P232S, P232T, P232V, P232W, P232Y, K233A, K233C, K233E, K233F, K233G, K233L, K233P, K233R, K233S, K233V, K233W, K233Y, G234A, G234C, G234D, G234E, G234K, G234L, G234Q, G234R, G234V, G234W, G234Y, G235C, G235F, G235H, G235I, G235K, G235M, G235Q, G235R, G235T, G235W, G235Y, K236A, K236D, K236E, K236G, K236L, K236M, K236P, K236R, K236S, K236T, K236W, K236Y, T237D, T237F, T237I, T237K, T237M, T237Q, T237R, T237S, T237V, T237Y, D238A, D238E, D238F, D238G, D238H, D238I, D238K, D238L, D238M, D238N, D238P, D238Q, D238R, A239C, A239E, A239G, A239I, A239K, A239T, A240C, A240E, A240L, A240P, A240Q, A240T, A240V, A240W, A240Y, I241T, G242K, G242L, G242M, G242P, G242T, G242Y, T243I, T243M, T243R, T243V, V244A, V244E, V244G, V244L, V244R, T245E, T245G, T245L, T245M, T245N, T245Q, T245R, T245S, T246D, T246E, T246G, T246K, T246V, A247D, A247E, A247K, A247N, A247P, A247Q, A247R, A247S, A247V, A247W, S248A, S248E, S248F, S248H, S248I, S248L, S248Q, S248T, S248Y, K249A, K249D, K249G, K249H, K249I, K249L, K249M, K249N, K249P, K249Q, K249S, K249T, K249V, K249Y, S250H, S250M, S250W, I251F, I251L, I251V, I251W, I251Y, A252C, A252E, A252F, A252H, A252I, A252P, A252R, A252S, A252W, A252Y, G254D, G254F, G254I, G254L, G254M, G254Q, G254R, G254W, A255C, A255F, A255K, A255L, A255M, A255S, A255T, A255W, A255Y, S256A, S256C, S256F, S256G, S256K, S256L, S256M, S256N, S256Q, S256R, S256V, S256W, S256Y, A257D, A257G, A257I, A257N, A257T, A257V, D258A, D258L, D258M, D258W, V259E, V259L, V259S, V259T, T260A, T260D, T260G, T260I, T260K, T260V, S261A, S261D, S261H, S261R, S261W, S261Y, T262D, T262E, T262F, T262G, T262H, T262L, T262P, T262W, I263A, I263C, I263G, I263L, I263S, I263V, A265G, A265I, A265K, A265R, A265S, A265V, S267A, S267D, S267K, S267M, S267N, S267P, S267Q, S267R, S267V, P268F, P268G, P268M, P268R, P268V, P268W, P268Y, K269G, K269R, K269V, K269Y, W271T, S272E, S272G, S272K, S272L, S272N, S272T, S272W, I273K, I273L, I273P, I273R, I273S, I273W, K274D, K274P, K274Q, K274R, N275K, N275M, N275V, N275W, N277F, N277K, N277R, L278A, L278G, L278H, L278I, L278K, L278M, L278P, L278Q, L278R, L278S, L278V, Y279M, Y279T, Y279W, T280A, T280D, T280E, T280F, T280H, T280M, T280Q, V281A, V281I, V281L, V281Q, R282E, R282F, R282H, R282I, R282K, R282N, R282S, R282T, R282V, R282W, T283M, T283R, T283V, E284A, E284D, E284F, E284H, E284L, E284M, E284N, E284Q, E284R, E284Y, V285H, V285I, V285T, L286A, L286C, L286D, L286F, L286N, L286R, L286T, L286W, L286Y, N287I, N287L, G288F, G288L, G288S, V291C, V291D, V291F, V291G, V291H, V291L, V291P, V291S, V291T, V291Y, L292A, L292D, L292E, L292H, L292Q, L292S, L292V, D293C, D293E, D293F, D293I, D293S, D293V, D293W, T294C, T294G, T294M, T294Q, T294S, Y295F, Y295G, Y295I, Y295L, Y295R, Y295S, Y295W, D296F, D296H, D296K, D296L, D296N, D296R, D296V, T297G, T297I, T297Q, T297R, T297S, E298G, E298I, E298L, E298M, E298R, E298T, Y299F, Y299S, F301C, F301L, R302E, R302I, R302K, R302M, R302N, R302T, W303A, W303C, W303D, W303F, W303S, W303T, T304D, T304E, T304I, T304K, T304P, T304S, T304W, G305E, G305F, G305L, G305M, G305N, G305P, G305T, G305W, F306D, F306K, F306L, F306S, D307A, D307E, D307F, D307G, D307L, D307M, D307N, D307Q, D307S, D307V, D307W, A308C, A308E, A308G, A308I, A308M, A308Y, S310A, S310C, S310G, S310H, S310I, S310L, S310M, S310N, S310R, S310V, G311E, G311F, G311L, G311Q, G311R, G311V, F312H, F312L, F312R, F312V, L314A, L314C, L314T, L314V, L314Y, N315S, G316A, G316C, G316N, G316R, G316S, G316W, G316Y, E317D, E317H, E317I, E317K, E317V, E317W, E317Y, K318C, K318E, K318F, K318G, K318H, K318L, K318M, K318N, K318P, K318Q, K318V, K320C, K320E, K320F, K320I, K320L, K320R, K320T, K320V, K320W, L321C, L321G, L321H, L321M, L321V, L321Y, K322C, K322F, K322I, K322N, K322P, K322Q, K322R, K322S, V324E, V324G, V324T, S325D, S325G, S325T, M326E, M326G, M326S, M326T, M326V, M326Y, H328C, H328D, H328F, H328G, H3281, H328L, H328M, H328R, H328T, D329S, D329T, G331V, A335C, A335G, A335L, A337D, A337E, A337G, A337L, A337Q, N338P, N338R, N338V, R340A, R340C, R340L, A341M, A341S, I342A, I342G, I342K, I342P, E343A, E343H, E343N, E343R, E343S, E343T, E343Y, R344G, R344L, R344Y, Q345F, Q345G, Q345K, Q345N, Q345S, V346A, V346C, V346F, V346I, V346L, V346S, E347A, E347C, E347D, E347F, E347I, E347R, E347S, I348A, I348D, I348G, I348M, I348Q, I348R, L349A, L349E, L349Q, L349S, L349V, Q350G, Q350N, Q350W, K351N, K351R, K351V, K351W, M352G, M352L, M352T, M352W, G353K, V354E, V354G, V354S, V354W, N355H, N355M, N355R, N355W, I357T, T359E, T359L, T360V, N362G, N362S, N362T, P363A, P363D, P363I, P363L, P363M, P363S, P363V, A364C, A364E, A364F, A364G, A364I, A364M, A364P, A364V, A364W, A365C, A365E, A365I, A365P, A365V, A365W, K366A, K366D, K366E, K366I, K366L, K366M, K366P, K366S, K366V, A367C, A367I, A367N, A367Q, L368A, L368E, L368Q, L368S, L368V, L368Y, I369A, I369D, I369E, I369G, I369K, I369M, I369R, I369V, I369W, D370C, D370L, D370Q, D370R, D370S, D370T, V371D, V371F, V371G, V371I, V371L, V371Q, V371S, C372P, N373G, N373L, N373R, E374K, E374L, E374R, K375D, K375I, K375N, K375Q, K375S, G376A, G376S, V377A, V377M, V377T, L378I, L378P, L378W, L378Y, V379A, V379C, V379M, V379N, V380M, V380P, V380S, E381A, E381C, E381G, E381L, E381Q, E381T, V383A, V383K, V383L, M386G, M386N, M386Q, M386S, M386V, W387H, W387L, N388A, N388E, N388L, N388R, R389A, R389C, R389E, R389K, R389M, R389N, R389Q, R389S, R389T, R389V, S390C, S390D, S390G, S390H, S390N, S390P, S390Q, S390T, S390V, K391E, N392D, G393A, G393E, G393N, G393R, G393S, G393V, N394L, T395A, T395C, T395F, T395H, T395I, T395M, T395N, T395Q, T395S, T395W, E396K, E396L, E396M, E396V, E396W, Y398M, Y398N, G399S, K400A, K400C, K400D, K400E, K400M, K400N, K400P, K400Q, K400S, K400T, K400V, W401F, W401H, W401K, W401L, W401M, W401R, F402T, F402W, F402Y, G403A, G403D, G403H, G403K, G403P, G403Q, G403S, G403T, G403V, G403Y, Q404F, Q404H, Q404L, Q404M, Q404P, Q404R, Q404S, Q404V, I406C, I406D, I406N, A407C, A407G, A407M, A407Q, A407S, A407T, A407W, G408D, G408I, G408M, G408N, G408W, D409N, D409W, N410C, N410R, N410Y, A411E, A411N, A411R, A411S, A411V, V412M, V412S, L413D, L413E, L413F, L413I, L413P, L413T, G414A, G414C, G414M, G414N, G414R, G414T, G414W, G415A, G415Q, G415R, D416I, D416M, D416R, D416T, D416Y, K417C, K417F, K417G, K417R, K417T, D418L, D418P, D418R, D418Y, E419M, E419R, E419W, W421L, W421Q, W421S, A422P, A422T, A422V, K423D, K423L, K423M, K423R, F424C, F424L, F424N, F424T, D425E, L426C, L426M, L426Q, T427D, T427F, T427G, T427K, T427M, T427P, T427Q, T427R, T427S, T427W, S428F, S428K, S428W, T429D, T429P, I430C, I430D, I430E, I430L, I430M, I430Q, I430S, I430T, I430W, N431D, N431E, N431G, N431L, N431M, N431R, N431V, N431Y, R432A, R432E, R432F, R432G, R432N, R432Q, R432V, R432Y, D433C, D433G, D433H, D433I, D433P, D433Q, D433W, R434L, R434M, R434N, R434P, R434S, R434T, R434V, N435E, N435F, N435H, N435K, N435L, N435M, N435R, N435V, N435W, A436C, A436D, A436E, A436G, A436I, A436L, A436M, A436Q, A436S, P437A, P437D, P437K, P437L, P437Q, P437R, P437S, P437V, P437W, S438G, V439C, V439E, V439G, V439I, V439K, V439Q, V439T, V439Y, I440C, I440D, I440F, I440K, I440P, I440R, I440S, I440T, I440V, I440W, M441A, M441E, M441G, M441Q, M441R, M441T, M441V, W442E, W442G, W442M, W442P, W442Q, W442R, S443C, S443D, S443G, S443M, S443Q, S443Y, L444C, L444D, L444E, L444F, L444G, L444H, L444K, L444Q, L444V, L444W, G445A, G445C, G445V, N446D, N446T, M448A, M448C, M448D, M448E, M448I, M448L, M448P, M448Q, M448S, M448V, M448W, M449D, M449E, M449F, M449T, M449V, M449W, M449Y, E450F, E450S, E450T, E450V, E450W, G451C, G451F, G451L, G451P, G451V, G451W, I452G, I452K, I452L, I452M, I452Q, I452S, I452V, S453C, S453F, S453G, S453H, S453L, S453M, S453N, S453P, S453Q, S453R, S453V, G454L, G454W, S455A, S455E, S455K, S455M, S455P, S455R, S455V, S455W, V456A, V456D, V456E, V456F, V456K, V456L, V456W, S457E, S457H, S457K, S457L, S457M, S457P, S457Q, S457T, S457V, F459A, F459C, F459E, F459G, F459N, F459R, F459S, F459T, F459W, A461D, A461G, A461M, A461N, A461Q, A461S, A461V, A461Y, T462C, T462E, T462F, T462L, T462M, T462S, S463G, S463K, S463Q, S463R, S463T, S463V, A464E, A464H, A464L, A464M, A464P, A464V, A464W, K465C, K465F, K465G, K465L, K465Q, K465R, K465V, K465W, K465Y, L466A, L466C, L466D, L466E, L466F, L466G, L466M, L466P, L466Q, L466S, L466V, L466Y, V467A, V467C, V467D, V467E, V467G, V467T, V467W, W469A, W469C, W469D, W469G, W469L, W469M, W469R, W469V, W469Y, T470E, T470L, T470M, T470Q, K471F, K471G, K471Q, K471W, K471Y, A472G, A472Y, A473E, A473M, A473P, D474A, D474C, D474E, D474K, D474M, D474R, D474W, S475E, S475F, S475Q, S475T, S475V, T476C, T476L, T476S, R477A, R477C, R477G, R477L, P478A, P478D, P478G, P478L, P478V, M479G, M479I, M479R, M479W, T480C, T480G, T480Q, K485E, K485R, K487A, K487C, K487F, K487G, K487N, K487S, K487W, A488C, A488G, A488H, A488L, A488N, A488S, A488V, N491A, N491E, N491W, E492A, E492W, S493E, S493G, S493H, S493L, S493M, S493Q, S493V, N494A, N494I, N494M, N494R, N494V, T495K, T495R, T495V, T495W, M496A, M496F, M496T, G497D, D498A, D498C, D498M, D498S, N499K, N499R, N499T, N499Y, L500A, L500E, L500N, L500V, T501C, T501G, T501M, A502L, A502Q, N503A, N503E, N503M, N503S, G504H, G504K, G504P, G505A, G505D, G505E, G505H, G505L, G505N, G505R, G505S, G505V, V506C, V506D, V506E, V506G, V506I, V506L, V506P, V506R, V506S, V506T, V506W, V507A, V507F, V507G, V507L, V507N, V507P, V507R, V507S, V507T, G508C, G508E, T509A, T509D, T509E, T509I, T509K, T509M, T509Q, T509S, T509V, T509Y, N510A, N510F, N510I, N510Q, Y511A, Y511K, S512C, S512E, S512F, S512G, S512I, S512M, S512Q, S512T, S512V, S512Y, G514F, G514L, G514N, G514P, G514R, N516C, N516E, N516G, N516, N516M, N516Q, N516S, N516T, N516V, Y517G, Y517I, Y517N, D518Q, D518Y, K519C, K519E, K519F, K519G, K519I, K519L, K519M, K519N, K519Q, K519S, K519T, I520A, I520C, I520G, I520H, I520M, I520N, I520Q, I520S, I520T, I520W, I520Y, R521A, R521C, R521K, R521N, R521Q, R521V, H524R, H524V, P525D, P525G, P525R, P525T, P525V, S526G, S526I, S526W, W527A, W527E, W527G, W527H, W527N, W527R, W527S, W527V, A528C, A528E, A528G, A528I, A528L, I529F, I529G, I529L, I529Y, Y530A, Y530M, G531E, G531S, G531T, T534A, T534I, T534Q, A535E, A535G, A535I, A535M, A537D, A537M, A537P, A537S, I538H, I538M, N539G, N539W, S540E, S540G, S540M, G542E, G542Q, G542S, G542T, I543V, I543W, N545G, N545I, N545Q, N545S, R546A, R546C, R546L, R546P, R546S, T547A, T547D, T547H, T547K, T547N, T547S, T548D, T548E, T548F, T548K, T548L, T548P, T548W, G549D, G549F, G549P, G549W, G550Q, G550R, G550S, A551D, A551I, A551Q, S553C, S553F, S553N, S553P, S553R, S553T, S553V, S554F, S554N, S554R, S554T, S554V, S554W, D555E, D555P, D555S, K556A, K556C, K556R, K556W, Q557F, Q557R, Q557S, L558E, L558H, L558I, L558P, T559A, T559G, T559I, T559Q, T559V, T559Y, S560P, S560V, Y561R, N563R, N563S, S564A, S564C, S564F, S564W, A565C, A565D, A565E, A565F, A565G, A565I, A565L, A565M, A565R, A565S, A565T, A565V, A565W, G567N, G567Q, G567V, A570G, A570K, A570L, A570M, A570S, A570T, A570W, V571W, A572S, A572W, S573G, S573K, S573Y, S574K, S574Q, S574V, S574W, A575D, A575M, A575V, W576A, W576F, W576V, W576Y, Y577G, Y577I, Y577L, Y577R, D578E, D578M, D578N, D578T, V579E, V579G, V579L, V579T, V580A, V580D, V580E, V580K, V580L, V580S, Q581F, Q581G, Q581P, Q581R, Q581S, Q581T, Q581Y, R582A, R582D, R582G, R582I, R582L, R582Y, D583V, D583W, F584E, F584I, F584W, V585I, V585M, V585Q, A586C, A586D, A586H, A586K, G587A, G587C, T588C, T588D, T588G, T588I, T588L, T588M, T588P, Y589A, Y589I, Y589Q, Y589V, Y589W, V590A, V590H, V590I, W591F, T592C, T592L, T592Q, T592S, G593C, G593I, F594C, F594I, F594L, F594M, D595E, D595Q, D595S, L597C, L597D, L597E, L597T, G598N, P600A, P600E, P600G, P600S, N604E, N604S, G605R, T606S, S608E, S608I, S608M, S608N, S608Q, S608T, S608V, G609A, G609H, G609L, G609N, G609R, G609S, A610D, A610F, A610M, A610T, V611K, G612N, G612T, G612V, S613A, P631A, P631D, P631G, P631H, P631S, P631V, P631Y, D633G, T634A, T634E, T634F, T634S, T634V, Y635R, Y636K, F637C, F637G, F637I, F637S, F637T, F637V, Y638A, Y638W, Q639D, Q639N, Q639R, S640C, S640D, Q641T, W642I, N643R, D644C, D644G, D644Y, D645S, D645V, V646C, V646L, V646N, V646R, V646S, H647G, H647V, T648C, L649V, H650E, H650F, H650R, I651F, I651T, I651V, L652C, L652D, L652V, L652W, P653Q, A654D, A654K, A654M, A654R, W655F, W655R, N656A, N656K, N656V, E657K, E657R, E657V, V659D, V659N, A661E, A661G, A661H, A661K, A661L, A661M, A661Q, A661W, K662H, K662S, K662V, K662W, K662Y, P669A, P669E, P669F, P669L, P669R, P669T, P669W, V670C, V672L, Y673E, Y673G, Y673I, Y673R, Y673S, Y673W, T674C, T674D, T674G, T674H, T674M, T674Q, D675A, D675E, D675P, D675Q, D675S, D675V, D675Y, A676C, A676E, A676G, A676K, A676L, A676P, A676S, A676W, A677E, A677G, A677L, A677R, A677T, A677V, A677Y, K678A, K678C, K678T, K678V, V679G, V679Q, V679S, V679T, V679Y, K680A, K680E, K680G, K680H, K680I, K680L, K680N, K680Q, K680S, K680V, K680W, L681E, L681F, L681G, L681M, L681S, L681T, Y682D, Y682E, Y682I, Y682M, Y682S, Y682V, Y682W, F683H, F683L, F683M, F683Q, F683R, F683W, T684A, T684D, T684G, T684L, T684R, T684S, T684V, P685A, P685E, P685I, P685L, P685W, K686A, K686E, K686I, K686M, K686T, K686V, G687A, G687K, G687N, G687P, G687Q, G687R, S688C, S688D, S688E, S688G, S688K, S688L, S688P, S688T, T689A, T689D, T689E, T689G, T689L, T689P, T689S, T689W, T689Y, E690D, E690M, E690P, E690T, E690V, L693A, L693M, L693P, I694L, I694W, G695C, G695K, G695L, G695R, G695W, E696A, E696L, E696R, K697A, K697E, K697G, K697R, K697V, K697W, S698C, S698D, S698E, S698I, S698L, S698M, S698P, S698Q, S698R, S698T, T700A, T700C, T700D, T700E, T700G, T700K, T700Y, K701A, K701D, K701E, K701G, K701H, K701L, K701M, K701P, K701S, K701W, T703E, T703I, T703K, T703W, T704M, T704R, T704Y, A705C, A705D, A705E, A705K, A705N, A705P, A705R, A705V, A705W, A706C, A706G, A706T, A706W, A706Y, Y708C, Y708F, Y708G, Y708K, Y708L, Y708P, Y708T, T709M, Y710C, Y710D, Y710E, Y710G, Y710M, Y710N, Y710T, Y710V, Y710W, Q711A, Q711D, Q711G, Q711L, Q711M, Q711T, Q711Y, V712G, V712M, V712P, V712Q, V712R, Y713A, Y713E, Y713G, Y713L, Y713Q, E714D, E714H, E714I, E714K, E714M, E714N, E714R, E714S, E714V, E714Y, G715K, K718A, K718L, K718Q, K718T, K718W, K718Y, S720A, S720M, S720R, S720Y, T721D, T721H, T721K, T721L, T721N, T721P, T721Q, T721S, T721V, T721W, T721Y, H723G, H723P, K724A, K724G, K724R, N725A, N725D, N725I, M726A, M726D, M726E, M726K, M726Q, M726V, M726W, Y727L, Y727T, L728C, L728K, L728Q, L728S, L728T, L728W, T729A, T729E, T729M, W730A, N731A, N731E, N731Q, N731S, N731Y, V732G, V732P, V732R, V732W, P733A, P733R, P733W, W734D, W734G, W734R, W734V, A735Q, A735R, A735S, A735T, A735W, E736S, G737F, G737K, G737N, G737Y, T738G, T738L, T738S, I739E, I739K, I739M, I739W, I739Y, S740D, S740E, S740F, S740H, S740L, A741I, A741P, A741S, A741V, E742D, E742M, E742Q, E742V, A743C, A743E, A743H, A743I, A743L, Y744A, Y744E, Y744I, Y744K, Y744L, Y744R, D745C, D745F, D745N, D745R, E746A, E746C, E746K, E746T, N747E, N747F, N747G, N747P, N747R, N748S, L750G, L750M, L750P, L750Q, L750S, I751C, I751H, I751Q, I751S, P752A, P752C, P752H, P752L, P752S, P752V, P752Y, E753Q, S755C, S755I, S755T, T756E, T756N, T756P, T756Q, T756S, T756W, E757A, G758A, G758V, N759D, N759R, N759S, N759V, A760G, A760N, A760P, A760Q, S761A, S761K, S761Q, V762D, V762G, V762K, V762W, T765A, T765P, T765R, T765W, G766M, G766S, A768H, A769F, A769G, A769I, A769N, A769V, A769Y, K770H, L771A, L771I, K772A, K772C, K772P, K772V, K772W, A773C, A773G, A773H, A773M, A773R, A773S, A773V, D774A, D774R, A775L, A775V, A775W, D776I, D776L, D776R, D776S, D776V, R777D, R777E, R777G, R777H, R777P, R777S, R777T, K778A, K778C, K778F, K778G, K778L, K778N, K778R, T779C, T779I, I780V, T781A, T781C, T781E, T781F, T781G, T781M, T781P, T781R, T781Y, A782C, A782E, A782K, A782N, A782P, A782Q, A782Y, D783A, D783C, D783E, D783R, G784A, G784F, G784L, G784S, G784T, K785C, K785I, K785S, K785V, K785W, K785Y, D786S, D786V, L787D, L787K, L787P, L787T, L787Y, S788A, S788G, S788I, Y789C, Y789D, Y789I, Y789V, I790A, I790C, I790F, I790R, I790V, E791A, E791D, E791F, E791M, E791S, E791T, E791V, E791W, V792C, V792G, V792L, V792S, V792Y, D793C, D793F, D793H, D793K, D793N, D793Y, V794C, V794D, V794L, V794Q, V794T, V794W, T795P, T795Y, D796M, D796Q, D796S, D796T, N798G, N798I, N798P, N798Q, G799D, G799K, G799L, G799M, G799Q, G799Y, H800A, H800F, H800G, H800L, H800S, H800V, I801C, I801E, I801W, V802E, V802I, V802P, V802S, V802Y, P803A, P803F, P803G, P803K, P803S, P803Y, D804E, D804G, D804K, D804N, D804S, A805C, A805F, A805G, A805I, A805N, A805P, A806F, A806I, A806Q, N807F, N807Q, N807V, N807W, R808C, R808F, R808G, R808I, R808N, R808P, R808Q, V809A, V809C, V809L, V809M, V809P, T810L, T810P, T810Q, T810R, T810Y, F811L, F811Y, D812E, D812F, D812I, D812Q, V813F, V813H, V813T, V813W, V813Y, K814G, K814H, K814I, K814L, K814P, G815A, G815F, G815M, G815P, G815V, A816C, A816D, A816F, A816I, A816N, A816V, A816W, G817C, G817H, G817I, G817N, G817S, K818D, K818F, K818L, K818Q, K818R, K818S, K818V, K818W, K818Y, L819F, L819W, V820C, V820F, V820I, V820K, V820R, V820W, G821A, G821C, G821E, G821F, G821I, G821K, G821M, G821N, G821V, G821Y, V822A, V822D, V822E, V822T, D823E, N824A, N824C, N824G, N824Q, G825A, S826A, S826F, S826G, S826I, S826L, S826R, S826W, S827C, S827Q, P828C, P828G, P828I, P828L, P828Y, D829C, D829I, D829S, D829T, D829V, H830E, H830G, H830M, H830P, H830Q, H830R, H830V, D831A, D831F, D831G, D831I, D831M, D831P, D831R, D831V, S832E, S832F, S832G, S832L, S832M, S832P, S832R, S832V, S832W, Y833C, Y833D, Y833E, Y833I, Y833K, Y833N, Y833P, Y833V, Q834F, Q834G, Q834M, A835D, A835E, A835F, A835H, A835K, A835W, D836C, D836E, D836H, D836Q, D836R, D836S, D836T, D836V, D836W, D836Y, N837D, N837F, N837G, N837H, N837L, N837P, N837T, R838D, R838F, R838G, R838K, R838M, R838N, R838S, R838W, A840G, A840I, A840P, A840V, F841C, F841D, F841I, F841K, F841W, S842A, S842L, S842M, G843A, G843C, K844A, K844G, K844L, K844W, K844Y, V845N, V845W, L846G, L846I, L846M, L846S, A847L, A847T, I848M, V849A, V849L, V849S, V849T, Q850C, Q850G, Q850I, Q850L, Q850T, Q850V, Q850Y, S851C, S851D, S851E, S851L, S851T, T852D, T852G, T852L, K853N, K853P, K853Q, K853V, E854C, E854I, E854M, E854R, A855K, A855V, A855Y, E857P, E857V, I858D, I858E, I858F, I858G, I858K, I858M, I858P, I858Q, I858Y, T859V, V860T, V860Y, T861F, T861I, T861Q, T861V, T861W, A862C, A862P, A862V, K863F, K863I, K863L, K863N, K863W, A864E, A864H, A864K, A864L, D865A, D865G, G866H, G866R, S870E, S870M, S870R, S870T, T871P, V872C, V872G, K873G, K873Y, I874G, A875R, T877A, V879P, V879S, P880S, G881W, T882A, T882M, T882R, S883L, T884A, E885V, K886E, K886L, K886V, K886W, T887A, T887D, T887F, T887G, T887N, T887R, T887V, V888A, V888D, V888G, R889G, Y893E, Y893G, S894D, S894G, R895M, N896M, Y897V, Y898T, V899G, K900E, K900G, T901G, T901Q, T901R, T901V, T901Y, G902A, G902D, G902F, G902L, G902P, G902Q, G902R, G902S, G902W, N903D, N903I, K904D, K904E, K904M, K904N, K904R, K904S, K904V, K904W, P905A, P905C, P905R, P905V, P905W, P905Y, L907F, L907S, L907Y, P908C, P908D, P908G, P908I, P908L, P908M, P908T, S909E, S909F, S909G, S909W, S909Y, D910C, D910I, D910S, D910W, V911A, V911S, E912A, E912K, E912L, E912T, E912V, V913G, V913Q, V913R, V913W, R914A, R914E, R914F, R914I, R914K, R914V, R914W, R914Y, Y915A, Y915C, Y915G, Y915I, Y915M, Y915Q, Y915V, S916G, D917C, D917E, D917F, D917L, D917M, D917Q, D917S, G918E, G918H, G918T, G918V, G918W, T919D, T919K, T919Q, T919W, T919Y, S920C, S920E, S920M, S920P, S920R, S920V, S920W, D921C, D921P, D921Q, D921V, R922A, R922G, R922M, R922V, R922W, Q923A, Q923C, Q923E, Q923L, Q923M, Q923V, Q923W, N924A, N924L, N924P, N924Q, N924S, N924W, V925A, V925C, V925E, V925G, V925K, V925N, V925S, V925W, T926G, T926P, T926R, T926S, T926V, T926W, W927C, W927G, W927P, D928A, D928E, D928H, D928L, D928Q, A929C, A929P, A929V, V930A, V930E, V930I, V930K, V930M, V930T, S931G, S931P, S931R, D932F, D932R, D932S, D932T, D932V, D933I, D933R, D933S, Q934S, Q934V, I935A, I935C, I935D, I935E, I935L, I935P, I935V, I935W, A936I, A936L, A936Q, A936R, A936Y, K937G, K937I, K937M, K937P, K937Q, K937R, K937V, A938C, A938H, A938N, A938T, A938V, A938W, G939D, G939K, S940C, S940E, S940M, S940R, S940T, S940V, S940W, F941C, F941M, F941W, S942A, S942E, S942K, S942L, S942P, S942T, S942V, V943A, V943G, V943H, V943Q, V943R, A944D, A944G, A944H, A944P, A944R, A944V, G945E, G945P, G945T, T946A, T946E, T946G, T946L, T946P, T946V, T946W, V947G, V947H, V947L, V947M, V947P, V947R, V947T, A948C, A948I, A948R, A948W, G949A, G949F, G949V, Q950D, Q950G, Q950K, Q950M, Q950W, K951D, K951G, K951P, K951Q, K951S, K951W, K951Y, I952H, I952Q, S953F, S953M, S953N, S953R, S953W, V954D, V954L, V954Q, V954S, V954T, R955A, R955C, R955E, R955K, R955Q, R955W, V956A, V956D, V956G, V956H, V956I, V956M, V956Q, V956W, T957D, T957S, T957W, M958D, M958I, M958K, I959A, I959L, I959S, I959V, I959Y, D960G, D960H, D960L, D960P, D960S, D960W, E961D, E961F, E961K, E961P, E961S, E961T, I962A, I962C, I962D, I962G, I962K, I962N, I962Q, I962T, G963C, G963E, G963L, G963P, A964C, A964E, A964H, L965C, L965E, L965G, L965K, L965M, L965P, L965Q, L965S, L965V, L965Y, L966A, L966G, L966H, L966K, L966N, L966P, L966Q, L966S, L966T, L966V, N967C, N967D, N967I, N967L, N967M, N967P, N967S, N967T, N967V, Y968G, Y968L, Y968Q, Y968V, S969C, S969D, S969G, S969H, S969I, S969L, S969M, S969P, S969Q, S969Y, A970I, A970L, P973C, P973D, P973K, P973N, P973Q, P973R, P973V, P973W, P973Y, V974C, V974E, V974G, V974N, V974T, V974Y, G975D, G975F, G975K, G975L, G975Q, G975V, T976D, T976F, T976G, T976K, T976L, T976P, T976S, T976Y, P977A, P977C, P977K, P977R, P977T, P977Y, A978F, A978G, A978M, A978N, A978P, A978R, A978S, A978Y, V979G, V979N, V979R, V979Y, L980A, L980F, L980H, L980I, L980K, L980N, L980Q, L980T, L980Y, P981L, P981M, R984P, R984S, P985E, P985F, P985H, P985K, P985L, P985W, A986C, A986E, A986F, A986I, A986K, A986L, A986M, A986N, A986S, A986W, V987A, V987C, V987F, V987I, V987K, V987L, V987Q, V987T, L988A, L988C, L988E, L988G, L988H, L988M, L988Q, L988R, L988S, L988V, L988Y, P989A, P989C, P989D, P989G, P989H, P989I, P989M, P989N, P989Q, P989W, D990F, D990P, D990S, D990W, G991C, G991F, G991H, G991K, G991P, G991Y, T992E, T992H, T992M, T992N, T992Y, V993D, V993G, V993N, V993S, T994I, T994S, T994V, S995E, S995L, S995R, S995V, A996Q, A996R, A996V, N997A, N997C, N997E, N997K, N997L, N997S, N997V, N997W, N997Y, F998M, F998W, A999F, A999G, A999L, A999M, A999R, A999S, V1000C, V1000L, V1000M, V1000N, V1000P, V1000W, W1002A, W1002D, W1002E, W1002H, W1002N, W1002P, W1002Q, W1002S, T1003F, T1003G, T1003L, T1003N, T1003P, T1003R, T1003S, T1003W, T1003Y, K1004D, K1004E, K1004F, K1004G, K1004H, K1004M, K1004P, K1004R, K1004S, K1004V, P1005I, P1005N, P1005Q, P1005V, P1005Y, A1006C, A1006I, A1006N, A1006P, A1006S, A1006V, A1006W, A1006Y, D1007C, D1007L, D1007P, D1007V, T1008G, V1009G, V1009S, Y1010A, Y1010P, Y1010R, Y1010T, N1011A, N1011S, N1011T, N1011W, T1012E, T1012H, T1012I, T1012Q, T1012Y, A1013D, A1013K, A1013Q, A1013T, A1013V, G1014E, G1014I, G1014L, G1014M, G1014V, G1014W, G1014Y, T1015A, T1015F, T1015G, T1015V, V1016C, V1016D, V1016P, K1017E, K1017G, V1018I, V1018K, V1018L, V1018M, V1018R, V1018S, V1018W, T1021C, T1021E, T1021F, T1021G, T1021K, T1021L, T1021S, T1021V, A1022H, A1022L, A1022S, A1022Y, T1023D, T1023M, T1023Q, T1023R, V1024E, V1024G, V1024H, V1024K, V1024N, V1024R, V1024S, V1024W, G1026E, G1026H, G1026L, G1026R, G1026S, G1026V, G1026Y, K1027C, K1027N, K1027Q, K1027R, K1027V, E1028G, E1028S, E1028T, F1029I, F1029K, F1029L, F1029P, F1029V, F1029W, F1029Y, K1030D, K1030F, K1030H, K1030L, K1030M, K1030W, V1031H, V1031K, V1031Y, A1033G, A1033S, A1033V, T1034G, T1034H, T1034N, T1034W, I1035D, I1035G, I1035Q, R1036G, R1036L, R1036T, R1036Y, V1037C, V1037F, V1037P, V1037Q, Q1038A, Q1038D, Q1038K, R1039S, R1039V, S1040A, S1040M, S1040N, S1040R, S1040W, Q1041P, V1042N, T1043F, T1043G, T1043N, T1043R, I1044A, I1044L, G1045S, S1046I, S1046M, S1047D, V1048C, V1048F, V1048G, V1048I, V1048M, V1048Q, G1050L, G1050S, G1050V, N1051A, N1051E, N1051K, A1052C, A1052K, A1052M, A1052P, A1052R, L1053A, L1053W, L1055R, L1055T, Q1057A, Q1057E, Q1057P, Q1057R, N1058R, N1058S, N1058V, N1058W, I1059W, P1060G, P1060N, P1060Q, P1060S, P1060T, D1062A, D1062F, D1062G, D1062I, D1062L, D1062M, D1062P, D1062S, K1063D, K1063M, Q1064C, Q1064M, Q1064R, Q1064T, Q1064V, S1065A, S1065C, S1065E, S1065G, S1065T, S1065W, D1066A, D1066G, D1066M, D1066V, D1066W, T1067G, T1067M, L1068C, L1068E, L1068P, L1068Q, L1068Y, A1070P, A1070T, I1071M, I1071R, I1071W, K1072E, K1072G, K1072P, K1072Q, K1072S, D1073F, D1073L, D1073M, D1073P, D1073W, G1074I, G1074L, G1074R, S1075C, S1075G, S1075I, S1075L, S1075V, T1076C, T1076E, T1076H, T1076Q, T1076S, T1077K, T1077L, T1077R, V1078D, V1078E, V1078L, V1078W, D1079G, D1079L, N1081D, N1081E, N1081G, T1082A, T1082C, T1082E, T1082F, T1082G, T1082K, T1082N, T1082S, G1083E, G1083F, G1083L, G1083P, G1083S, G1084C, G1084M, G1084V, G1084W, G1084Y, G1085A, G1085P, G1085R, G1085S, A1086H, A1086K, A1086Q, A1086R, A1086T, N1087A, N1087E, N1087I, N1087R, N1087V, N1087W, P1088D, P1088E, P1088G, P1088R, P1088W, S1089C, S1089E, S1089G, S1089K, S1089Q, S1089R, S1089V, A1090F, A1090G, A1090I, A1090K, W1091A, W1091E, W1091G, W1091H, W1091T, W1091V, W1091Y, T1092A, T1092E, T1092G, T1092K, T1092Q, T1092S, T1092V, N1093A, N1093G, N1093L, N1093P, N1093Q, N1093T, N1093V, W1094D, W1094E, W1094P, W1094R, W1094T, A1095P, A1095R, A1095T, A1095W, Y1096A, Y1096D, Y1096H, Y1096L, Y1096R, S1097D, S1097E, S1097K, S1097L, S1097T, S1097W, K1098D, K1098F, K1098G, K1098Q, K1098S, A1099C, A1099D, A1099F, A1099S, A1099V, A1099W, G1100D, G1100E, G1100H, G1100M, G1100N, G1100T, H1101K, H1101L, H1101Q, H1101R, H1101V, N1102E, N1102F, N1102H, N1102K, N1102L, N1102Q, N1102R, N1102T, T1103A, T1103E, T1103H, T1103S, T1103W, A1104I, A1104K, A1104R, E1105L, E1105S, I1106T, I1106V, T1107C, T1107M, T1107R, T1107S, F1108D, F1108K, F1108L, F1108T, F1108W, E1109A, E1109D, E1109L, E1109W, A1111G, A1111S, E1113D, E1113G, E1113P, E1113V, Q1114E, Q1114I, Q1114L, Q1114R, Q1114S, Q1114T, Q1114V, Q1115A, Q1115K, Q1115L, Q1115P, Q1115T, Q1115W, L1116D, L1116G, L1116H, L1116K, L1116V, L1116W, G1117E, G1117I, G1117M, G1117R, G1117S, G1117T, G1117V, G1117W, Q1118A, Q1118M, Q1118S, Q1118T, Q1118W, I1119D, I1119E, I1119G, I1119N, I1119S, V1120N, V1120S, V1120T, M1121G, M1121K, M1121N, M1121P, M1121S, M1121V, M1121Y, Y1122A, Y1122C, Y1122I, Y1122K, Y1122R, Y1122V, Y1122W, F1123E, F1123H, F1123R, F1123T, F1124E, F1124R, F1124V, F1124W, R1125D, R1125E, R1125F, R1125K, R1125T, R1125V, R1125W, D1126H, D1126K, D1126L, D1126R, S1127F, S1127I, S1127K, S1127M, S1127Q, S1127T, S1127W, N1128A, N1128C, N1128R, N1128S, N1128T, N1128W, A1129E, A1129L, A1129N, A1129Q, A1129R, A1129V, V1130A, V1130G, V1130P, V1130R, V1130S, R1131A, R1131N, R1131Q, R1131S, R1131W, F1132E, F1132K, F1132M, F1132P, F1132Q, F1132T, P1133D, P1133G, P1133L, P1133Q, P1133R, P1133V, D1134E, D1134G, D1134L, A1135E, A1135K, A1135L, A1135M, A1135S, A1135W, A1135Y, G1136A, G1136E, G1136P, G1136Q, G1136T, K1137A, K1137C, K1137G, K1137L, K1137P, K1137Q, K1137R, K1137S, K1137T, K1137V, T1138R, T1138Y, K1139A, K1139L, K1139R, K1139T, I1140A, I1140C, I1140G, I1140L, I1140M, I1140P, I1140R, I1140T, Q1141A, Q1141C, Q1141G, Q1141K, Q1141N, Q1141P, Q1141T, Q1141W, I1142E, I1142R, I1142S, I1142W, I1142Y, S1143G, A1144C, A1144D, A1144E, A1144N, A1144P, A1144S, A1144T, A1144V, A1144W, D1145E, D1145R, D1145T, G1146A, G1146C, G1146D, G1146K, G1146L, G1146R, G1146V, K1147A, K1147G, K1147T, K1147V, N1148H, N1148I, N1148K, N1148P, N1148Q, N1148R, N1148S, N1148T, N1148W, W1149C, W1149G, W1149I, W1149K, W1149N, W1149Q, W1149S, W1149T, W1149V, W1149Y, T1150G, T1150K, T1150P, D1151C, D1151G, D1151R, D1151T, D1151W, L1152A, L1152C, L1152E, L1152Q, L1152W, A1153E, A1153G, A1153K, A1153L, A1154C, A1154D, A1154E, A1154G, A1154R, A1154S, T1155E, T1155L, T1155Q, T1155R, T1157V, T1157W, I1158R, I1158S, I1158W, A1159C, A1159E, A1159I, A1159P, A1159R, A1159V, Q1161A, Q1161P, Q1161S, E1162A, E1162C, E1162D, E1162F, E1162I, E1162N, E1162Q, E1162T, E1162W, E1162Y, R1166D, R1166K, R1166Q, V1167A, V1167C, V1167L, V1167P, V1167R, K1168L, K1168Q, K1168R, K1168W, P1169M, P1169R, P1169S, Y1170E, Y1170K, Y1170M, Y1170Q, Y1170R, Y1170V, T1171A, T1171G, T1171M, T1171Q, T1171R, T1171S, Y1172D, Y1172E, Y1172H, Y1172I, Y1172K, Y1172L, Y1172S, Y1172V, D1173A, D1173E, D1173F, D1173G, D1173K, D1173L, D1173P, D1173R, D1173T, D1173W, F1174D, F1174P, F1174Q, F1174R, F1174S, F1174T, F1174V, F1174W, A1175G, A1175I, A1175N, A1175Q, A1175S, A1175V, A1175Y, V1177N, V1177P, V1177S, V1177T, G1178M, G1178Q, G1178S, G1178T, A1179L, A1179P, A1179Q, A1179W, T1180A, T1180G, T1180I, T1180L, T1180M, T1180Q, T1180S, T1180Y, F1181E, F1181L, F1181V, V1182M, V1184C, V1184E, V1184K, V1184L, V1184P, V1184Q, V1184Y, T1185Q, T1185V, V1186S, N1188V, N1188W, A1189C, A1189K, A1189T, A1189V, D1190R, D1190S, D1190T, D1190Y, T1191E, T1191L, T1192H, T1192P, T1193G, P1194A, P1194E, P1194G, P1194W, S1195G, V1197A, V1198E, C1199D, C1199T, L1202A, L1202C, T1203E, T1203K, E1204G, E1204S, I1205V, K1208R, K1208V, K1208W, T1209A, T1209C, T1209E, T1209K, T1209N, T1209Q, T1209R, T1209W, A1210D, A1210E, A1210G, A1210K, A1210L, A1210Q, A1210R, A1210T, A1210W, T1211C, T1211D, T1211E, T1211G, T1211H, T1211K, T1211P, T1211Q, T1211R, T1211S, T1211V, K1213A, K1213D, K1213S, K1213T, K1213W, F1214A, F1214E, F1214K, F1214L, F1214P, F1214R, F1214S, F1214V, V1215D, V1215E, V1215K, V1215L, V1215Q, V1215S, V1215W, N1217A, N1217D, N1217E, N1217F, N1217P, N1217R, N1217S, N1217T, T1218A, T1218C, T1218E, T1218G, T1218Q, T1218S, T1218V, T1218W, S1219A, S1219E, S1219F, S1219I, S1219K, S1219R, S1219V, A1220C, A1220G, A1220L, A1220P, A1220R, A1220V, A1221D, A1221G, A1221K, A1221L, A1221R, A1221V, A1221W, L1222A, L1222C, L1222E, L1222F, L1222Q, L1222R, L1222V, L1222W, S1223C, S1223F, S1223G, S1223K, S1223L, S1223V, S1224A, S1224D, S1224G, S1224L, S1224M, S1224P, S1224R, S1224W, L1225C, L1225D, L1225E, L1225F, L1225G, L1225K, L1225P, L1225T, L1225V, L1225W, T1226A, T1226G, T1226M, T1226P, T1226R, T1226S, T1226V, T1226Y, V1227A, V1227C, V1227D, V1227E, V1227G, V1227L, V1227P, V1227Q, V1227S, N1228A, N1228D, N1228F, N1228K, N1228L, N1228T, T1230F, T1230H, T1230I, T1230K, T1230L, T1230P, T1230R, T1230S, T1230W, K1231F, K1231G, K1231L, K1231M, K1231P, K1231S, K1231W, V1232E, V1232K, V1232Q, V1232R, V1232S, V1232T, V1232W, S1233P, S1233W, D1234G, D1234K, D1234R, D1234V, S1235D, S1235E, S1235G, S1235L, S1235P, S1235R, S1235W, S1235Y, V1236A, V1236C, V1236G, V1236I, V1236P, V1236Q, V1236R, L1237D, L1237E, L1237R, L1237V, L1237W, A1238D, A1238E, A1238K, A1238L, A1238N, A1238P, A1238R, A1238S, A1238T, A1239D, A1239P, A1239R, G1240D, G1240L, G1240N, G1240Q, G1240S, G1240T, G1240W, S1241D, S1241G, S1241I, S1241L, S1241M, S1241P, Y1242C, Y1242E, Y1242K, Y1242R, Y1242S, Y1242W, N1243C, N1243L, N1243M, N1243P, N1243Q, N1243S, N1243T, N1243V, N1243W, T1244A, T1244D, T1244E, T1244G, T1244L, T1244Q, T1244S, T1244V, T1244W, A1246F, A1246M, A1246N, A1246P, A1246Q, A1246R, A1246S, A1246T, I1247A, I1247G, I1247M, I1247Q, I1247R, I1247S, I1247T, I1247V, I1247W, I1248A, I1248G, I1248K, I1248L, I1248R, I1248S, I1248Y, A1249E, A1249G, A1249H, A1249I, A1249R, A1249T, A1249V, D1250I, D1250K, D1250S, D1250T, D1250W, D1250Y, V1251I, V1251T, V1251W, K1252D, K1252G, K1252V, K1252W, A1253P, A1253V, E1254F, E1254G, E1254H, E1254L, E1254R, E1254V, G1255H, G1255M, G1255S, G1255V, E1256G, E1256M, E1256N, E1256R, E1256V, E1256W, G1257F, G1257K, G1257L, G1257Q, G1257R, G1257W, N1258C, N1258G, N1258H, N1258K, N1258S, A1259K, A1259L, A1259W, V1261I, V1261L, V1261P, V1261Q, V1261R, V1261T, T1262A, T1262F, T1262M, T1262Q, T1262R, V1263E, V1263G, V1263Q, V1263R, V1263T, V1263W, L1264A, L1264E, L1264H, L1264R, L1264S, L1264Y, P1265C, P1265K, P1265L, P1265R, P1265S, P1265V, P1265W, A1266F, A1266L, A1266P, A1266S, A1266V, H1267A, H1267E, H1267F, N1269A, N1269E, N1269K, N1269R, N1269S, N1269T, N1269W, V1270D, V1270E, V1270G, V1270I, V1270L, V1270T, V1270W, I1271A, I1271H, I1271Q, R1272E, R1272F, R1272M, R1272P, R1272V, V1273R, I1274F, I1274M, I1274R, T1275A, T1275L, T1275W, E1276R, E1276W, S1277L, S1277T, S1277W, E1278Q, E1278R, D1279G, D1279I, D1279R, D1279T, D1279V, D1279W, H1280C, H1280E, H1280G, H1280V, H1280W, V1281F, V1281I, V1281S, V1281W, T1282D, T1282L, T1282V, R1283A, R1283D, R1283E, R1283P, R1283W, K1284G, T1285A, T1285E, T1285F, T1285G, T1285M, T1285R, T1285Y, F1286A, F1286E, F1286P, F1286R, F1286S, F1286T, T1287C, T1287K, T1287L, T1287M, T1287Q, T1287R, T1287S, T1287W, I1288A, I1288D, I1288F, I1288G, I1288K, N1289A, N1289Q, N1289T, L1290A, L1290G, L1290R, L1290V, L1290W, G1291K, G1291P, G1291V, G1291W, G1291Y, T1292G, T1292L, T1292Y, E1293G, E1293K, E1293L, E1293S, E1293V, Q1294E, Q1294L, Q1294P, Q1294W, E1295K, F1296A, F1296G, F1296I, P1297F, A1298Y, D1301I, E1302G, E1302R, E1302S, R1303S, D1304A or D1304V.

147. The lactase variant of the preceding embodiment, wherein the variant has a decreased or an increased galactose inhibition, preferably measured as a galactose inhibition improvement factor (GI-IF) of at least 1.1 or at most 0.9, compared to the lactase of SEQ ID NO: 1.

148. The lactase variant of any of the preceding embodiments which has an amino acid sequence which is at least 80%, 85%, 90%, or 95%, preferably at least 97%, 98%, or 99%, more preferably at least 99.5%, 99.7%, 99.8% or 99.9%, but less than 100%, identical to SEQ ID NO: 1.

149. The lactase variant of any of the preceding embodiments wherein the variant has lactase activity.

150. The lactase variant of any of the preceding embodiments, wherein the variant is a variant of a lactase obtained from Bifidobacterium, preferably Bifidobacterium bifidum.

151. The lactase variant of any of the preceding embodiments wherein the variant has a galactose inhibition improvement factor (GI-IF) of at least 1.1 or at most 0.9, compared to the lactase of SEQ ID NO: 1, and wherein the GI-IF is calculated as described in Example 2.

152. The lactase variant of any of the preceding embodiments wherein the variant has a length of 800-2000 amino acids, such as 1200-2000 amino acids, preferably 1200-1500 amino acids, more preferably 1250-1400 amino acids or 1250-1350 amino acids, even more preferably 1280-1320 amino acids, such as 1300-1320 amino acids or 1300-1310 amino acids, most preferably 1300-1305, such as 1302-1304 amino acids.

153. The lactase variant of any of the preceding embodiments which has an amino acid sequence which is at least 90% identical to SEQ ID NO: 1 and which has a length of 1200-2000 amino acids.

154. The lactase variant of any of the preceding embodiments which has an amino acid sequence which is at least 90% identical to SEQ ID NO: 1 and which has a length of 1200-1500 amino acids.

155. The lactase variant of any of the preceding embodiments which has an amino acid sequence which is at least 90% identical to SEQ ID NO: 1 and which has a length of 1250-1350 amino acids.

156. The lactase variant of any of the preceding embodiments which has an amino acid sequence which is at least 90% identical to SEQ ID NO: 1 and which has a length of 1300-1310 amino acids.

157. The lactase variant of any of the preceding embodiments which has an amino acid sequence which is at least 95% identical to SEQ ID NO: 1 and which has a length of 1200-2000 amino acids.

158. The lactase variant of any of the preceding embodiments which has an amino acid sequence which is at least 95% identical to SEQ ID NO: 1 and which has a length of 1200-1500 amino acids.

159. The lactase variant of any of the preceding embodiments which has an amino acid sequence which is at least 95% identical to SEQ ID NO: 1 and which has a length of 1250-1350 amino acids.

160. The lactase variant of any of the preceding embodiments which has an amino acid sequence which is at least 95% identical to SEQ ID NO: 1 and which has a length of 1300-1310 amino acids.

161. The lactase variant of any of the preceding embodiments which has an amino acid sequence which is at least 99% identical to SEQ ID NO: 1 and which has a length of 1200-2000 amino acids.

162. The lactase variant of any of the preceding embodiments which has an amino acid sequence which is at least 99% identical to SEQ ID NO: 1 and which has a length of 1200-1500 amino acids.

163. The lactase variant of any of the preceding embodiments which has an amino acid sequence which is at least 99% identical to SEQ ID NO: 1 and which has a length of 1250-1350 amino acids.

164. The lactase variant of any of the preceding embodiments which has an amino acid sequence which is at least 99% identical to SEQ ID NO: 1 and which has a length of 1300-1310 amino acids.

165. The lactase variant of the preceding embodiment which comprises the same number of amino acid residues as the lactase of SEQ ID NO: 1.

166. The lactase variant of any of the preceding embodiments wherein the variant has 1-50 substitutions, preferably 1-30, 1-20, 1-10 or 1-5, such as 1, 2, 3, 4, 5, 6, 7, 8, 9, 10, 11, 12, 13, 14, 15, 16, 17, 18, 19 or 20 substitutions, compared to the lactase of SEQ ID NO: 1.

167. The lactase variant of any of the preceding embodiments which is an isolated lactase variant.

168. The lactase variant of any of the preceding embodiments which has a ratio of beta-galactosidase activity to transgalactosylating activity of at least 2, preferably at least 3, such as at least 4, more preferably at least 5, such as at least 10, where said ratio is measured as [Galactose]/([Glucose]−[Galactose]) after incubation of the enzyme in cow's milk at 37° C. for 3 hours.

169. An isolated polynucleotide encoding the variant of any of the preceding embodiments.

170. A nucleic acid construct or expression vector comprising the polynucleotide of the preceding embodiment.

171. A recombinant host cell transformed with the polynucleotide of embodiment 169.

172. A method of producing a lactase variant, comprising:
a. cultivating the host cell of the preceding embodiment under conditions suitable for expression of the variant; and
b. recovering the variant 173. A whole broth formulation or cell culture composition comprising the variant of any of embodiments 1-168.

174. A method for producing a dairy product comprising
a) providing a milk-based substrate comprising lactose; and
b) treating said substrate with the variant of any of embodiments 1-168.

175. The method of the preceding embodiment wherein step b) results in a lactose reduction of at least 70%, preferably at least 80%, more preferably at least 90%, even more preferably at least 95% or at least 98%.

176. The method of any of the two preceding embodiments, further comprising a step
c) fermenting said substrate with a microorganism, wherein the dairy product is a fermented dairy product

177. The method of the preceding embodiment wherein step b) and step c) are performed essentially at the same time.

178. The method of any of the two preceding embodiments wherein the treatment with the lactase variant results in a lactose reduction of at least 50%, preferably at least 70% or at least 90%, compared to a similar method without addition of the lactase variant.

179. The method of any of the five preceding embodiments wherein the lactose content in the dairy product is below 2% w/v, preferably below 1%, more preferably below 0.5% or below 0.1%, and most preferably below 0.05% or below 0.01% w/v.

180. Use of the variant of any of embodiments 1-168 for production of a dairy product.

181. The use according to the preceding embodiment wherein the dairy product is a low-lactose dairy product having a lactose content of below 2% w/v, preferably below 1%, more preferably below 0.5% or below 0.1%, and most preferably below 0.05% or below 0.01% w/v.

182. A liquid composition comprising the variant of any of embodiments 1-168 and 20-70% glycerol.

183. A method for obtaining a lactase variant, comprising:
a. introducing into a parent lactase having an amino acid sequence which is at least 80% identical to SEQ ID NO: 1 an alteration, preferably a substitution, at one or more positions corresponding to positions 1, 2, 3, 4, 5, 6, 7, 9, 10, 11, 12, 13, 14, 15, 16, 17, 19, 20, 21, 22, 23, 24, 25, 26, 27, 28, 29, 30, 31, 32, 33, 34, 35, 36, 37, 38, 39, 40, 41, 42, 43, 44, 45, 47, 48, 49, 51, 53, 54, 55, 57, 58, 59, 60, 61, 62, 63, 64, 66, 67, 68, 70, 71, 73, 74, 75, 76, 77, 78, 79, 80, 81, 83, 85, 86, 87, 88, 89, 90, 91, 92, 93, 94, 95, 96, 97, 98, 100, 101, 102, 104, 105, 106, 107, 108, 109, 110, 111, 112, 113, 114, 115, 116, 117, 118, 119, 120, 121, 122, 123, 124, 125, 126, 128, 129, 130, 131, 132, 133, 135, 136, 137, 138, 139, 140, 141, 142, 146, 148, 149, 150, 151, 153, 154, 155, 157, 158, 159, 160, 161, 162, 164, 165, 166, 167, 168, 169, 170, 171, 172, 173, 174, 175, 176, 177, 178, 179, 180, 181, 182, 183, 184, 185, 186, 187, 188, 189, 190, 191, 193, 194, 195, 196, 197, 198, 199, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 254, 255, 256, 257, 258, 259, 260, 261, 262, 263, 264, 265, 266, 267, 268, 269, 270, 271, 272, 273, 274, 275, 277, 278, 279, 280, 281, 282, 283, 284, 285, 286, 287, 288, 291, 292, 293, 294, 295, 296, 297, 298, 299, 301, 302, 303, 304, 305, 306, 307, 308, 309, 310, 311, 312, 314, 315, 316, 317, 318, 320, 321, 322, 324, 325, 326, 328, 329, 331, 335, 337, 338, 340, 341, 342, 343, 344, 345, 346, 347, 348, 349, 350, 351, 352, 353, 354, 355, 357, 359, 360, 362, 363, 364, 365, 366, 367, 368, 369, 370, 371, 372, 373, 374, 375, 376, 377, 378, 379, 380, 381, 383, 386, 387, 388, 389, 390, 391, 392, 393, 394, 395, 396, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 485, 487, 488, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 534, 535, 537, 538, 539, 540, 542, 543, 545, 546, 547, 548, 549, 550, 551, 553, 554, 555, 556, 557, 558, 559, 560, 561, 563, 564, 565, 567, 570, 571, 572, 573, 574, 575, 576, 577, 578, 579, 580, 581, 582, 583, 584, 585, 586, 587, 588, 589, 590, 591, 592, 593, 594, 595, 597, 598, 600, 604, 605, 606, 607, 608, 609, 610, 611, 612, 613, 614, 615, 616, 619, 620, 621, 624, 625, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 659, 661, 662, 667, 669, 670, 672, 673, 674, 675, 676, 677, 678, 679, 680, 681, 682, 683, 684, 685, 686, 687, 688, 689, 690, 691, 692, 693, 694, 695, 696, 697, 698, 700, 701, 703, 704, 705, 706, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 765, 766, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 857, 858, 859, 860, 861, 862, 863, 864, 865, 866, 867, 870, 871, 872, 873, 874, 875, 877, 879, 880, 881, 882, 883, 884, 885, 886, 887, 888, 889, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 973, 974, 975, 976, 977, 978, 979, 980, 981, 982, 984, 985, 986, 987, 988, 989, 990, 991, 992, 993, 994, 995, 996, 997, 998, 999, 1000, 1001, 1002, 1003, 1004, 1005, 1006, 1007, 1008, 1009, 1010, 1011, 1012, 1013, 1014, 1015, 1016, 1017, 1018, 1021, 1022, 1023, 1024, 1026, 1027, 1028, 1029, 1030, 1031, 1033, 1034, 1035, 1036, 1037, 1038, 1039, 1040, 1041, 1042, 1043, 1044, 1045, 1046, 1047, 1048, 1050, 1051, 1052, 1053, 1054, 1055, 1057, 1058, 1059, 1060, 1061, 1062, 1063, 1064, 1065, 1066, 1067, 1068, 1069, 1070, 1071, 1072, 1073, 1074, 1075, 1076, 1077, 1078, 1079, 1081, 1082, 1083, 1084, 1085, 1086, 1087, 1088, 1089, 1090, 1091, 1092, 1093, 1094, 1095, 1096, 1097, 1098, 1099, 1100, 1101, 1102, 1103, 1104, 1105, 1106, 1107, 1108, 1109, 1111, 1113, 1114, 1115, 1116, 1117, 1118, 1119, 1120, 1121, 1122, 1123, 1124, 1125, 1126, 1127, 1128, 1129, 1130, 1131, 1132, 1133, 1134, 1135, 1136, 1137, 1138, 1139, 1140, 1141, 1142, 1143, 1144, 1145, 1146, 1147, 1148, 1149, 1150, 1151, 1152, 1153, 1154, 1155, 1157, 1158, 1159, 1160, 1161, 1162, 1165, 1166, 1167, 1168, 1169, 1170, 1171, 1172, 1173, 1174, 1175, 1177, 1178, 1179, 1180, 1181, 1182, 1183, 1184, 1185, 1186, 1188, 1189, 1190, 1191, 1192, 1193, 1194, 1195, 1197, 1198, 1199, 1200, 1202, 1203, 1204, 1205, 1208, 1209, 1210, 1211, 1213, 1214, 1215, 1216, 1217, 1218, 1219, 1220, 1221, 1222, 1223, 1224, 1225, 1226, 1227, 1228, 1229, 1230, 1231, 1232, 1233, 1234, 1235, 1236, 1237, 1238, 1239, 1240, 1241, 1242, 1243, 1244, 1246, 1247, 1248, 1249, 1250, 1251, 1252, 1253, 1254, 1255, 1256, 1257, 1258, 1259, 1261, 1262, 1263, 1264, 1265, 1266, 1267, 1269, 1270, 1271, 1272, 1273, 1274, 1275, 1276, 1277, 1278, 1279, 1280, 1281, 1282, 1283, 1284, 1285, 1286, 1287, 1288, 1289, 1290, 1291, 1292, 1293, 1294, 1295, 1296, 1297, 1298, 1301, 1302, 1303 or 1304 of the polypeptide of SEQ ID NO: 1, and
b. recovering the variant.

184. The method of the preceding embodiment, wherein the positions are the positions listed in any one of embodiments 3, 5, 7, 9, 11, 13, 15, 17 19, 21, 23, 25, 27, 29, 31, 33, 35, 37, 39, 41, 43, 45, 47, 49, 51, 53, 55, 57, 59, 61, 63, 65, 67, 69 or 71.

185. The method of embodiment 183, wherein one or more of the following substitutions are introduced into the parent lactase, where each position corresponds to a position in SEQ ID NO: 1: V1D, V1F, V1H, V1K, V1L, V1R, E2D, E2G, E2Q, E2V, D3A, D3H, D3I, D3N, D3S, D3V, D3W, A4C, A4G, A4H, A4I, A4L, A4M, A4P, T5A, T5D, T5F, T5K, T5R, T5S, T5V, R6A, R6D, R6F, R6G, R6H, R6L, R6M, R6P, R6S, R6W, S7A, S7D, S7I, S7L, S7N, S7P, S7T, S7W, S9G, S9H, S9P, S9R, S9W, T10G, T10K, T10L, T10P, T10R, T10S, T10W, T11L, T11P, Q12L, Q12R, Q12V, Q12Y, M13C, M13D, M13E, M13F, M13H, M13K, M13R, M13W, S14A, S14G, S14H, S14L, S14T, S14V, S14Y, S15C, S15F, S15I, S15K, S15L, S15P, S15R, S15T, S15V, S15W, S15Y, T16A, T16C, T16I, T16N, T16S, P17A, P17C, P17D, P17E, P17I, P17L, P17N, P17R, P17S, P17T, V19A, V19F, V19G, V19I, V19K, V19L, V19N, V19S, V19W, V20C, V20F, V20G, V20I, V20K, V20L, V20M, V20N, V20P, V20Q, V20R, V20T, V20W, Y21A, Y21C, Y21D, Y21F, Y21G, Y21H, Y21M, Y21P, Y21R, Y21T, S22A, S22E, S22F, S22G, S22L, S22M, S22N, S22R, S22T, S22W, S23A, S23C, S23D, S23L, S23M, S23R, A24F, A24G, A24L, A24R, A24T, A24W, V25D, V25E, V25F, V25G, V25H, V25K, V25L, V25M, V25Q, V25R, V25S, V25T, V25W, D26C, D26I, D26L, D26M, D26T, D26V, S27A, S27C, S27F, S27G, S27H, S27P, S27Y, K28C, K28G, K28I, K28L, K28R, K28S, K28V, K28W, Q29D, Q29F, Q29G, Q29L, Q29M, Q29R, Q29S, Q29V, Q29W, N30A, N30G, N30H, N30M, N30P, N30V, N30W, N30Y, R31E, R31G, R31I, R31M, R31V, T32M, T32Q, T32R, T32S, S33C, S33E, S33H, S33K, S33N, S33Q, S33R, S33V, D34C, D34E, D34F, D34G, D34H, D34L, D34S, D34W, D34Y, F35A, F35C, F35E, F35G, F35K, F35N, F35T, F35V, D36H, D36Q, A37N, A37Q, N38C, N38G, N38S, W39G, W39S, K40C, K40D, K40F, K40G, K40I, K40M, K40N, K40P, K40W, F41A, F41C, F41G, F41I, F41Q, F41S, F41Y, M42E, M42N, M42T, L43A, L43C, L43G, L43I, L43S, L43T, L43V, S44C, S44M, S44N, S44Y, D45A, D45L, D45P, D45V, V47K, V47R, Q48S, A49C, A49D, A49H, A49R, A49S, A49T, A49V, D51G, D51I, D51K, D51M, D51P, D51V, A53C, A53G, A53L, A53R, A53S, A53T, A53V, A53W, F54M, F54S, D55C, D55F, D55G, D55H, D55M, D55N, D55P, D55S, D55V, S57A, S57C, S57E, S57G, A58D, A58G, A58I, A58M, A58N, A58Q, A58R, A58T, W59D, W59I, W59K, W59L, W59N, W59P, W59V, Q60A, Q60C, Q60E, Q60F, Q60G, Q60K, Q60L, Q60M, Q60R, Q60S, Q60V, Q60Y, Q61K, Q61P, Q61S, V62G, V62N, V62S, V62T, V62W, D63G, D63L, D63P, D63S, D63V, L64E, L64G, H66C, H66L, H66R, H66T, H66W, H66Y, D67E, Y68P, Y68V, I70A, I70H, I70K, I70P, I70R, T71C, T71E, T71G, T71H, T71K, T71L, T71N, T71P, T71Q, T71R, T71S, K73A, K73D, K73F, K73G, K73Q, K73V, Y74G, Y74K, Y74T, Y74W, S75G, S75L, S75Q, S75R, S75V, Q76G, Q76I, Q76K, Q76M, Q76P, Q76S, Q76V, Q76Y, S77C, S77D, S77E, S77F, S77G, S77H, S77I, S77K, S77L, S77M, S77R, S77T, S77V, S77W, S77Y, N78C, N78E, N78F, N78K, N78Q, N78R, N78S, N78T, E79H, E79Q, E79S, E79T, E79W, A80K, E81A, E81Q, A83E, A83T, L85A, L85C, L85D, L85F, L85M, L85N, L85S, L85V, L85W, P86E, P86G, P86H, P86N, P86Q, P86R, P86V, P86W, P86Y, G87A, G87D, G87E, G87N, G87Q, G88A, G88F, G88I, G88M, G88Q, G88S, T89C, T89G, T89H, T89K, T89L, T89M, T89N, T89P, T89W, T89Y, G90A, G90C, G90D, G90L, G90S, G90T, G90V, W91E, W91L, W91P, W91Q, W91R, W91S, W91Y, Y92F, Y92H, Y92I, Y92M, Y92N, Y92S, Y92T, Y92V, Y92W, R93A, R93D, R93F, R93H, R93I, R93L, R93N, R93T, R93V, R93Y, K94A, K94C, K94G, K94R, K94S, K94T, K94V, S95A, S95C, S95D, S95E, S95G, S95I, S95L, S95Q, S95R, F96A, F96C, F96I, F96K, F96L, F96M, F96P, F96S, F96V, F96W, T97F, T97S, T97V, I98C, I98H, I98S, I98W, R100T, D101A, D101P, D101V, L102A, L102G, L102M, L102P, L102S, G104C, K105D, K105Q, K105R, K105W, K105Y, R106K, R106P, R106V, R106W, I107A, I107F, I107G, I107Q, I107S, A108E, A108S, A108V, I109M, I109T, N110A, N110F, N110S, N110T, N110V, N110W, F111A, F111C, F111L, F111Q, F111V, D112A, D112F, D112G, D112T, G113A, G113S, V114F, V114G, V114M, V114R, Y115E, M116A, M116C, M116D, M116W, M116Y, N117K, N117R, N117T, N117W, A118K, A118P, A118Y, T119A, T119G, T119L, V120A, V120K, W121C, W121D, W121R, W121T, W121V, W121Y, F122A, F122M, F122S, F122Y, N123P, G124E, G124M, G124Q, G124R, V125D, V125E, V125I, K126E, K126V, G128A, G128D, T129E, T129V, H130A, H130C, H130Q, H130S, H130T, P131K, P131L, P131S, Y132C, Y132E, Y132S, G133E, S135E, S135P, S135V, P136R, P136Y, F137A, F137C, F137D, F137G, F137L, F137P, S138A, S138D, S138G, S138H, S138L, S138M, S138R, S138V, F139A, F139E, F139Q, F139W, D140G, D140L, D140V, L141G, L141T, T142E, T142S, T142V, K146A, G148H, G148K, G148T, G149E, G149H, G149I, G149M, G149Q, G149Y, E150A, E150C, E150G, E150L, E150N, E150R, N151L, I153A, I153Y, V154E, V154I, V154K, V154L, V154M, V154S, V155F, V157A, V157G, V157L, V157P, V157Q, V157S, E158G, E158H, E158K, E158Q, E158V, N159D, N159H, N159S, N159T, R160G, L161E, L161K, L161M, L161S, L161W, P162F, P162G, P162N, P162T, P162W, S164A, R165H, W166S, Y167A, Y167C, S168C, G169A, G169C, G169D, G169S, S170L, S170Q, G171C, G171F, G171T, I172G, I172K, I172P, I172Q, Y173A, Y173H, Y173M, Y173P, Y173S, Y173W, R174E, R174K, D175E, D175Y, V176E, V176K, V176T, T177A, T177C, T177E, T177K, T177L, L178I, L178Q, L178W, T179A, T179C, T179D, T179H, T179I, T179K, T179L, T179N, T179P, T179S, V180A, V180C, V180D, V180E, V180G, V180M, T181A, T181D, T181F, T181K, T181R, D182F, D182L, D182S, G183W, V184F, V184H, V184P, V184Q, V184R, V184S, V184W, H185G, H185L, H185R, V186A, V186E, V186G, V186N, G187A, G187D, G187H, N188E, N188R, N188S, N188V, N188W, N189A, N189E, G190C, G190F, G190H, G190Q, G190V, V191C, V191T, V191W, V191Y, I193N, I193Q, I193T, I193V, K194A, K194I, K194L, K194R, T195A, T195E, T195M, T195S, T195W, P196A, P196H, P196I, P196M, P196S, P196W, S197A, S197C, S197E, S197K, S197L, S197P, L198E, L198F, L198H, L198I, L198K, L198R, L198V, L198W, A199E, A199F, A199K, A199P, A199R, A199T, Q201C, Q201E, Q201I, Q201K, Q201M, Q201V, N202A, N202D, N202F, N202G, N202K, N202L, N202M, N202Q, N202R, N202S, N202T, N202W, G203C, G203K, G203M, G203Q, G203R, G203S, G203V, G203W, G203Y, G204A, G204C, G204D, G204K, G204R, G204S, G204Y, N205E, N205G, N205H, N205L, N205P, N205W, N205Y, V206A, V206C, V206D, V206F, V206G, V206I, V206K, V206Q, V206R, V206S, V206T, T207A, T207C, T207G, T207I, T207K, T207L, T207M, T207N, T207Q, T207R, T207W, M208A, M208S, M208T, N209C, N209D, N209G, N209K, N209L, N209Q, N209R, N209V, L210A, L210C, L210F, L210G, L210H, L210I, L210Q, L210R, L210S, L210T, L210V, T211A, T211D, T211E, T211F, T211K, T211N, T211Q, T211R, T211S, T212A, T212C, T212E, T212F, T212G, T212H, T212K, T212L, T212M, T212S, T212W, K213A, K213C, K213D, K213F, K213I, K213L, K213M, K213N, K213Q, K213R, K213S, K213T, K213V, K213Y, V214A, V214C, V214T, V214W, A215D, A215E, A215F, A215I, A215K, A215L, A215Q, A215R, A215S, A215V, N216D, N216T, N216V, D217F, D217G, D217L, D217M, D217T, D217V, T218D, T218G, T218H, K219A, K219C, K219F, K219H, K219M, A220C, A220G, A220I, A220L, A220M, A220T, A220V, A220W, A221C, A221D, A221E, A221L, A221N, A221R, A221V, A221Y, A222D, A222I, A222L, A222P, A222R, A222W, A222Y, N223A, N223E, N223F, N223G, N223K, N223L, N223M, N223R, N223S, N223T, N223V, N223W, I224G, I224Q, T225A, T225G, T225L, L226C, L226M, L226Q, K227T, Q228N, Q228R, T229A, T229C, T229D, T229G, T229H, T229M, T229N, T229Q, T229R, T229V, V230F, V230L, V230M, V230Q, V230R, V230S, F231A, F231E, F231G, F231I, F231K, F231L, F231Q, F231S, F231V, F231W, F231Y, P232G, P232H, P232L, P232M, P232R, P232S, P232T, P232V, P232W, P232Y, K233A, K233C, K233E, K233F, K233G, K233L, K233P, K233R, K233S, K233V, K233W, K233Y, G234A, G234C, G234D, G234E, G234K, G234L, G234Q, G234R, G234V, G234W, G234Y, G235C, G235F, G235H, G235l, G235K, G235M, G235Q, G235R, G235T, G235W, G235Y, K236A, K236D, K236E, K236G, K236L, K236M, K236P, K236R, K236S, K236T, K236W, K236Y, T237D, T237F, T237I, T237K, T237M, T237Q, T237R, T237S, T237V, T237Y, D238A, D238E, D238F, D238G, D238H, D238I, D238K, D238L, D238M, D238N, D238P, D238Q, D238R, A239C, A239E, A239G, A239I, A239K, A239T, A240C, A240E, A240L, A240P, A240Q, A240T, A240V, A240W, A240Y, I241T, G242K, G242L, G242M, G242P, G242T, G242Y, T243I, T243M, T243R, T243V, V244A, V244E, V244G, V244L, V244R, T245E, T245G, T245L, T245M, T245N, T245Q, T245R, T245S, T246D, T246E, T246G, T246K, T246V, A247D, A247E, A247K, A247N, A247P, A247Q, A247R, A247S, A247V, A247W, S248A, S248E, S248F, S248H, S248I, S248L, S248Q, S248T, S248Y, K249A, K249D, K249G, K249H, K249I, K249L, K249M, K249N, K249P, K249Q, K249S, K249T, K249V, K249Y, S250H, S250M, S250W, I251F, I251L, I251V, I251W, I251Y, A252C, A252E, A252F, A252H, A252I, A252P, A252R, A252S, A252W, A252Y, G254D, G254F, G254I, G254L, G254M, G254Q, G254R, G254W, A255C, A255F, A255K, A255L, A255M, A255S, A255T, A255W, A255Y, S256A, S256C, S256F, S256G, S256K, S256L, S256M, S256N, S256Q, S256R, S256V, S256W, S256Y, A257D, A257G, A257I, A257N, A257T, A257V, D258A, D258L, D258M, D258W, V259E, V259L, V259S, V259T, T260A, T260D, T260G, T260I, T260K, T260V, S261A, S261D, S261H, S261R, S261W, S261Y, T262D, T262E, T262F, T262G, T262H, T262L, T262P, T262W, I263A, I263C, I263G, I263L, I263S, I263V, T264F, T264G, T264K, T264L, T264M, T264P, T264Q, T264R, T264S, T264Y, A265G, A265I, A265K, A265R, A265S, A265V, A266D, A266E, A266G, A266K, A266L, A266M, A266P, A266Q, A266S, A266T, S267A, S267D, S267K, S267M, S267N, S267P, S267Q, S267R, S267V, P268F, P268G, P268M, P268R, P268V, P268W, P268Y, K269G, K269R, K269V, K269Y, L270D, L270M, L270N, L270R, L270V, W271T, S272E, S272G, S272K, S272L, S272N, S272T, S272W, I273K, I273L, I273P, I273R, I273S, I273W, K274D, K274P, K274Q, K274R, N275K, N275M, N275V, N275W, N277F, N277K, N277R, L278A, L278G, L278H, L278I, L278K, L278M, L278P, L278Q, L278R, L278S, L278V, Y279M, Y279T, Y279W, T280A, T280D, T280E, T280F, T280H, T280M, T280Q, V281A, V281I, V281L, V281Q, R282E, R282F, R282H, R282I, R282K, R282N, R282S, R282T, R282V, R282W, T283M, T283R, T283V, E284A, E284D, E284F, E284H, E284L, E284M, E284N, E284Q, E284R, E284Y, V285H, V285I, V285T, L286A, L286C, L286D, L286F, L286N, L286R, L286T, L286W, L286Y, N287I, N287L, G288F, G288L, G288S, V291C, V291D, V291F, V291G, V291H, V291L, V291P, V291S, V291T, V291Y, L292A, L292D, L292E, L292H, L292Q, L292S, L292V, D293C, D293E, D293F, D293I, D293S, D293V, D293W, T294C, T294G, T294M, T294Q, T294S, Y295F, Y295G, Y295I, Y295L, Y295R, Y295S, Y295W, D296F, D296H, D296K, D296L, D296N, D296R, D296V, T297G, T297I, T297Q, T297R, T297S, E298G, E298I, E298L, E298M, E298R, E298T, Y299F, Y299S, F301C, F301L, R302E, R302I, R302K, R302M, R302N, R302T, W303A, W303C, W303D, W303F, W303S, W303T, T304D, T304E, T304I, T304K, T304P, T304S, T304W, G305E, G305F, G305L, G305M, G305N, G305P, G305T, G305W, F306D, F306K, F306L, F306S, D307A, D307E, D307F, D307G, D307L, D307M, D307N, D307Q, D307S, D307V, D307W, A308C, A308E, A308G, A308I, A308M, A308Y, T309C, T309D, T309E, T309I, T309K, T309M, T309S, T309V, S310A, S310C, S310G, S310H, S310I, S310L, S310M, S310N, S310R, S310V, G311E, G311F, G311L, G311Q, G311R, G311V, F312H, F312L, F312R, F312V, L314A, L314C, L314T, L314V, L314Y, N315S, G316A, G316C, G316N, G316R, G316S, G316W, G316Y, E317D, E317H, E317I, E317K, E317V, E317W, E317Y, K318C, K318E, K318F, K318G, K318H, K318L, K318M, K318N, K318P, K318Q, K318V, K320C, K320E, K320F, K320I, K320L, K320R, K320T, K320V, K320W, L321C, L321G, L321H, L321M, L321V, L321Y, K322C, K322F, K322I, K322N, K322P, K322Q, K322R, K322S, V324E, V324G, V324T, S325D, S325G, S325T, M326E, M326G, M326S, M326T, M326V, M326Y, H328C, H328D, H328F, H328G, H3281, H328L, H328M, H328R, H328T, D329S, D329T, G331V, A335C, A335G, A335L, A337D, A337E, A337G, A337L, A337Q, N338P, N338R, N338V, R340A, R340C, R340L, A341M, A341S, I342A, I342G, I342K, I342P, E343A, E343H, E343N, E343R, E343S, E343T, E343Y, R344G, R344L, R344Y, Q345F, Q345G, Q345K, Q345N, Q345S, V346A, V346C, V346F, V346I, V346L, V346S, E347A, E347C, E347D, E347F, E347I, E347R, E347S, I348A, I348D, I348G, I348M, I348Q, I348R, L349A, L349E, L349Q, L349S, L349V, Q350G, Q350N, Q350W, K351N, K351R, K351V, K351W, M352G, M352L, M352T, M352W, G353K, V354E, V354G, V354S, V354W, N355H, N355M, N355R, N355W, I357T, T359E, T359L, T360V, N362G, N362S, N362T, P363A, P363D, P363I, P363L, P363M, P363S, P363V, A364C, A364E, A364F, A364G, A364I, A364M, A364P, A364V, A364W, A365C, A365E, A365I, A365P, A365V, A365W, K366A, K366D, K366E, K366I, K366L, K366M, K366P, K366S, K366V, A367C, A367I, A367N, A367Q, L368A, L368E, L368Q, L368S, L368V, L368Y, I369A, I369D, I369E, I369G, I369K, I369M, I369R, I369V, I369W, D370C, D370L, D370Q, D370R, D370S, D370T, V371D, V371F, V371G, V371I, V371L, V371Q, V371S, C372P, N373G, N373L, N373R, E374K, E374L, E374R, K375D, K375I, K375N, K375Q, K375S, G376A, G376S, V377A, V377M, V377T, L378I, L378P, L378W, L378Y, V379A, V379C, V379M, V379N, V380M, V380P, V380S, E381A, E381C, E381G, E381L, E381Q, E381T, V383A, V383K, V383L, M386G, M386N, M386Q, M386S, M386V, W387H, W387L, N388A, N388E, N388L, N388R, R389A, R389C, R389E, R389K, R389M, R389N, R389Q, R389S, R389T, R389V, S390C, S390D, S390G, S390H, S390N, S390P, S390Q, S390T, S390V, K391E, N392D, G393A, G393E, G393N, G393R, G393S, G393V, N394L, T395A, T395C, T395F, T395H, T395I, T395M, T395N, T395Q, T395S, T395W, E396K, E396L, E396M, E396V, E396W, Y398M, Y398N, G399S, K400A, K400C, K400D, K400E, K400M, K400N, K400P, K400Q, K400S, K400T, K400V, W401F, W401H, W401K, W401L, W401M, W401R, F402T, F402W, F402Y, G403A, G403D, G403H, G403K, G403P, G403Q, G403S, G403T, G403V, G403Y, Q404F, Q404H, Q404L, Q404M, Q404P, Q404R, Q404S, Q404V, A405C, A405E, A405H, A405K, A405P, A405R, A405T, A405V, I406C, I406D, I406N, A407C, A407G, A407M, A407Q, A407S, A407T, A407W, G408D, G408I, G408M, G408N, G408W, D409N, D409W, N410C, N410R, N410Y, A411E, A411N, A411R, A411S, A411V, V412M, V412S, L413D, L413E, L413F, L413I, L413P, L413T, G414A, G414C, G414M, G414N, G414R, G414T, G414W, G415A, G415Q, G415R, D416I, D416M, D416R, D416T, D416Y, K417C, K417F, K417G, K417R, K417T, D418L, D418P, D418R, D418Y, E419M, E419R, E419W, T420E, T420F, T420G, T420K, T420R, T420V, W421L, W421Q, W421S, A422P, A422T, A422V, K423D, K423L, K423M, K423R, F424C, F424L, F424N, F424T, D425E, L426C, L426M, L426Q, T427D, T427F, T427G, T427K, T427M, T427P, T427Q, T427R, T427S, T427W, S428F, S428K, S428W, T429D, T429P, I430C, I430D, I430E, I430L, I430M, I430Q, I430S, I430T, I430W, N431D, N431E, N431G, N431L, N431M, N431R, N431V, N431Y, R432A, R432E, R432F, R432G, R432N, R432Q, R432V, R432Y, D433C, D433G, D433H, D433I, D433P, D433Q, D433W, R434L, R434M, R434N, R434P, R434S, R434T, R434V, N435E, N435F, N435H, N435K, N435L, N435M, N435R, N435V, N435W, A436C, A436D, A436E, A436G, A436I, A436L, A436M, A436Q, A436S, P437A, P437D, P437K, P437L, P437Q, P437R, P437S, P437V, P437W, S438G, V439C, V439E, V439G, V439I, V439K, V439Q, V439T, V439Y, I440C, I440D, I440F, I440K, I440P, I440R, I440S, I440T, I440V, I440W, M441A, M441E, M441G, M441Q, M441R, M441T, M441V, W442E, W442G, W442M, W442P, W442Q, W442R, S443C, S443D, S443G, S443M, S443Q, S443Y, L444C, L444D, L444E, L444F, L444G, L444H, L444K, L444Q, L444V, L444W, G445A, G445C, G445V, N446D, N446T, M448A, M448C, M448D, M448E, M448I, M448L, M448P, M448Q, M448S, M448V, M448W, M449D, M449E, M449F, M449T, M449V, M449W, M449Y, E450F, E450S, E450T, E450V, E450W, G451C, G451F, G451L, G451P, G451V, G451W, I452G, I452K, I452L, I452M, I452Q, I452S, I452V, S453C, S453F, S453G, S453H, S453L, S453M, S453N, S453P, S453Q, S453R, S453V, G454L, G454W, S455A, S455E, S455K, S455M, S455P, S455R, S455V, S455W, V456A, V456D, V456E, V456F, V456K, V456L, V456W, S457E, S457H, S457K, S457L, S457M, S457P, S457Q, S457T, S457V, G458A, G458C, G458D, G458F, G458L, G458P, G458Q, G458S, G458V, G458W, F459A, F459C, F459E, F459G, F459N, F459R, F459S, F459T, F459W, P460C, P460M, P460Q, P460W, P460Y, A461D, A461G, A461M, A461N, A461Q, A461S, A461V, A461Y, T462C, T462E, T462F, T462L, T462M, T462S, S463G, S463K, S463Q, S463R, S463T, S463V, A464E, A464H, A464L, A464M, A464P, A464V, A464W, K465C, K465F, K465G, K465L, K465Q, K465R, K465V, K465W, K465Y, L466A, L466C, L466D, L466E, L466F, L466G, L466M, L466P, L466Q, L466S, L466V, L466Y, V467A, V467C, V467D, V467E, V467G, V467T, V467W, A468D, A468E, A468F, A468K, A468L, A468P, A468S, A468V, A468W, W469A, W469C, W469D, W469G, W469L, W469M, W469R, W469V, W469Y, T470E, T470L, T470M, T470Q, K471F, K471G, K471Q, K471W, K471Y, A472G, A472Y, A473E, A473M, A473P, D474A, D474C, D474E, D474K, D474M, D474R, D474W, S475E, S475F, S475Q, S475T, S475V, T476C, T476L, T476S, R477A, R477C, R477G, R477L, P478A, P478D, P478G, P478L, P478V, M479G, M479I, M479R, M479W, T480C, T480G, T480Q, K485E, K485R, K487A, K487C, K487F, K487G, K487N, K487S, K487W, A488C, A488G, A488H, A488L, A488N, A488S, A488V, N491A, N491E, N491W, E492A, E492W, S493E, S493G, S493H, S493L, S493M, S493Q, S493V, N494A, N494I, N494M, N494R, N494V, T495K, T495R, T495V, T495W, M496A, M496F, M496T, G497D, D498A, D498C, D498M, D498S, N499K, N499R, N499T, N499Y, L500A, L500E, L500N, L500V, T501C, T501G, T501M, A502L, A502Q, N503A, N503E, N503M, N503S, G504H, G504K, G504P, G505A, G505D, G505E, G505H, G505L, G505N, G505R, G505S, G505V, V506C, V506D, V506E, V506G, V506I, V506L, V506P, V506R, V506S, V506T, V506W, V507A, V507F, V507G, V507L, V507N, V507P, V507R, V507S, V507T, G508C, G508E, T509A, T509D, T509E, T509I, T509K, T509M, T509Q, T509S, T509V, T509Y, N510A, N510F, N510I, N510Q, Y511A, Y511K, S512C, S512E, S512F, S512G, S512I, S512M, S512Q, S512T, S512V, S512Y, D513C, D513G, D513K, D513L, D513M, D513P, D513Q, D513R, D513W, G514F, G514L, G514N, G514P, G514R, A515C, A515D, A515E, A515F, A515G, A515K, A515P, A515R, A515S, N516C, N516E, N516G, N516I, N516M, N516Q, N516S, N516T, N516V, Y517G, Y517I, Y517N, D518Q, D518Y, K519C, K519E, K519F, K519G, K519I, K519L, K519M, K519N, K519Q, K519S, K519T, I520A, I520C, I520G, I520H, I520M, I520N, I520Q, I520S, I520T, I520W, I520Y, R521A, R521C, R521K, R521N, R521Q, R521V, T522G, T522I, T522N, T523A, T523M, H524R, H524V, P525D, P525G, P525R, P525T, P525V, S526G, S526I, S526W, W527A, W527E, W527G, W527H, W527N, W527R, W527S, W527V, A528C, A528E, A528G, A528I, A528L, I529F, I529G, I529L, I529Y, Y530A, Y530M, G531E, G531S, G531T, T534A, T534I, T534Q, A535E, A535G, A535I, A535M, A537D, A537M, A537P, A537S, I538H, I538M, N539G, N539W, S540E, S540G, S540M, G542E, G542Q, G542S, G542T, I543V, I543W, N545G, N545I, N545Q, N545S, R546A, R546C, R546L, R546P, R546S, T547A, T547D, T547H, T547K, T547N, T547S, T548D, T548E, T548F, T548K, T548L, T548P, T548W, G549D, G549F, G549P, G549W, G550Q, G550R, G550S, A551D, A551I, A551Q, S553C, S553F, S553N, S553P, S553R, S553T, S553V, S554F, S554N, S554R, S554T, S554V, S554W, D555E, D555P, D555S, K556A, K556C, K556R, K556W, Q557F, Q557R, Q557S, L558E, L558H, L558I, L558P, T559A, T559G, T559I, T559Q, T559V, T559Y, S560P, S560V, Y561R, N563R, N563S, S564A, S564C, S564F, S564W, A565C, A565D, A565E, A565F, A565G, A565I, A565L, A565M, A565R, A565S, A565T, A565V, A565W, G567N, G567Q, G567V, A570G, A570K, A570L, A570M, A570S, A570T, A570W, V571W, A572S, A572W, S573G, S573K, S573Y, S574K, S574Q, S574V, S574W, A575D, A575M, A575V, W576A, W576F, W576V, W576Y, Y577G, Y577I, Y577L, Y577R, D578E, D578M, D578N, D578T, V579E, V579G, V579L, V579T, V580A, V580D, V580E, V580K, V580L, V580S, Q581F, Q581G, Q581P, Q581R, Q581S, Q581T, Q581Y, R582A, R582D, R582G, R582I, R582L, R582Y, D583V, D583W, F584E, F584I, F584W, V585I, V585M, V585Q, A586C, A586D, A586H, A586K, G587A, G587C, T588C, T588D, T588G, T588I, T588L, T588M, T588P, Y589A, Y589I, Y589Q, Y589V, Y589W, V590A, V590H, V590I, W591F, T592C, T592L, T592Q, T592S, G593C, G593I, F594C, F594I, F594L, F594M, D595E, D595Q, D595S, L597C, L597D, L597E, L597T, G598N, P600A, P600E, P600G, P600S, N604E, N604S, G605R, T606S, G607A, G607Q, G607S, S608E, S608I, S608M, S608N, S608Q, S608T, S608V, G609A, G609H, G609L, G609N, G609R, G609S, A610D, A610F, A610M, A610T, V611K, G612N, G612T, G612V, S613A, W614A, W614F, W614P, P615L, P615S, P615V, S616A, S616W, N619A, N619I, N619L, N619S, S620G, Y621W, I624A, I624V, V625A, V625E, V625F, V625M, V625Y, T627F, T627G, T627K, T627Q, A628C, A628D, A628N, G629T, F630A, F630C, F630D, F630G, F630S, F630Y, P631A, P631D, P631G, P631H, P631S, P631V, P631Y, K632C, K632D, K632G, K632T, D633G, T634A, T634E, T634F, T634S, T634V, Y635R, Y636K, F637C, F637G, F637I, F637S, F637T, F637V, Y638A, Y638W, Q639D, Q639N, Q639R, S640C, S640D, Q641T, W642I, N643R, D644C, D644G, D644Y, D645S, D645V, V646C, V646L, V646N, V646R, V646S, H647G, H647V, T648C, L649V, H650E, H650F, H650R, I651F, I651T, I651V, L652C, L652D, L652V, L652W, P653Q, A654D, A654K, A654M, A654R, W655F, W655R, N656A, N656K, N656V, E657K, E657R, E657V, V659D, V659N, A661E, A661G, A661H, A661K, A661L, A661M, A661Q, A661W, K662H, K662S, K662V, K662W, K662Y, N667L, N667R, P669A, P669E, P669F, P669L, P669R, P669T, P669W, V670C, V672L, Y673E, Y673G, Y673I, Y673R, Y673S, Y673W, T674C, T674D, T674G, T674H, T674M, T674Q, D675A, D675E, D675P, D675Q, D675S, D675V, D675Y, A676C, A676E, A676G, A676K, A676L, A676P, A676S, A676W, A677E, A677G, A677L, A677R, A677T, A677V, A677Y, K678A, K678C, K678T, K678V, V679G, V679Q, V679S, V679T, V679Y, K680A, K680E, K680G, K680H, K680I, K680L, K680N, K680Q, K680S, K680V, K680W, L681E, L681F, L681G, L681M, L681S, L681T, Y682D, Y682E, Y682I, Y682M, Y682S, Y682V, Y682W, F683H, F683L, F683M, F683Q, F683R, F683W, T684A, T684D, T684G, T684L, T684R, T684S, T684V, P685A, P685E, P685I, P685L, P685W, K686A, K686E, K686I, K686M, K686T, K686V, G687A, G687K, G687N, G687P, G687Q, G687R, S688C, S688D, S688E, S688G, S688K, S688L, S688P, S688T, T689A, T689D, T689E, T689G, T689L, T689P, T689S, T689W, T689Y, E690D, E690M, E690P, E690T, E690V, K691E, K691H, K691N, K691P, K691R, K691S, R692G, R692H, R692I, R692L, R692P, R692S, R692T, R692V, R692W, L693A, L693M, L693P, I694L, I694W, G695C, G695K, G695L, G695R, G695W, E696A, E696L, E696R, K697A, K697E, K697G, K697R, K697V, K697W, S698C, S698D, S698E, S698I, S698L, S698M, S698P, S698Q, S698R, S698T, T700A, T700C, T700D, T700E, T700G, T700K, T700Y, K701A, K701D, K701E, K701G, K701H, K701L, K701M, K701P, K701S, K701W, T703E, T703I, T703K, T703W, T704M, T704R, T704Y, A705C, A705D, A705E, A705K, A705N, A705P, A705R, A705V, A705W, A706C, A706G, A706T, A706W, A706Y, Y708C, Y708F, Y708G, Y708K, Y708L, Y708P, Y708T, T709M, Y710C, Y710D, Y710E, Y710G, Y710M, Y710N, Y710T, Y710V, Y710W, Q711A, Q711D, Q711G, Q711L, Q711M, Q711T, Q711Y, V712G, V712M, V712P, V712Q, V712R, Y713A, Y713E, Y713G, Y713L, Y713Q, E714D, E714H, E714I, E714K, E714M, E714N, E714R, E714S, E714V, E714Y, G715K, A716C, A716G, A716L, A716M, A716P, A716R, A716T, A716V, D717C, D717G, D717L, D717S, K718A, K718L, K718Q, K718T, K718W, K718Y, D719E, D719L, D719P, D719S, D719T, D719V, S720A, S720M, S720R, S720Y, T721D, T721H, T721K, T721L, T721N, T721P, T721Q, T721S, T721V, T721W, T721Y, A722Q, A722T, A722V, H723G, H723P, K724A, K724G, K724R, N725A, N725D, N725I, M726A, M726D, M726E, M726K, M726Q, M726V, M726W, Y727L, Y727T, L728C, L728K, L728Q, L728S, L728T, L728W, T729A, T729E, T729M, W730A, N731A, N731E, N731Q, N731S, N731Y, V732G, V732P, V732R, V732W, P733A, P733R, P733W, W734D, W734G, W734R, W734V, A735Q, A735R, A735S, A735T, A735W, E736S, G737F, G737K, G737N, G737Y, T738G, T738L, T738S, I739E, I739K, I739M, I739W, I739Y, S740D, S740E, S740F, S740H, S740L, A741I, A741P, A741S, A741V, E742D, E742M, E742Q, E742V, A743C, A743E, A743H, A743I, A743L, Y744A, Y744E, Y744I, Y744K, Y744L, Y744R, D745C, D745F, D745N, D745R, E746A, E746C, E746K, E746T, N747E, N747F, N747G, N747P, N747R, N748S, R749H, R749M, R749S, R749T, L750G, L750M, L750P, L750Q, L750S, I751C, I751H, I751Q, I751S, P752A, P752C, P752H, P752L, P752S, P752V, P752Y, E753Q, G754H, G754I, G754P, G754R, S755C, S755I, S755T, T756E, T756N, T756P, T756Q, T756S, T756W, E757A, G758A, G758V, N759D, N759R, N759S, N759V, A760G, A760N, A760P, A760Q, S761A, S761K, S761Q, V762D, V762G, V762K, V762W, T765A, T765P, T765R, T765W, G766M, G766S, A768H, A769F, A769G, A769I, A769N, A769V, A769Y, K770H, L771A, L771I, K772A, K772C, K772P, K772V, K772W, A773C, A773G, A773H, A773M, A773R, A773S, A773V, D774A, D774R, A775L, A775V, A775W, D776I, D776L, D776R, D776S, D776V, R777D, R777E, R777G, R777H, R777P, R777S, R777T, K778A, K778C, K778F, K778G, K778L, K778N, K778R, T779C, T779I, I780V, T781A, T781C, T781E, T781F, T781G, T781M, T781P, T781R, T781Y, A782C, A782E, A782K, A782N, A782P, A782Q, A782Y, D783A, D783C, D783E, D783R, G784A, G784F, G784L, G784S, G784T, K785C, K785I, K785S, K785V, K785W, K785Y, D786S, D786V, L787D, L787K, L787P, L787T, L787Y, S788A, S788G, S788I, Y789C, Y789D, Y789I, Y789V, I790A, I790C, I790F, I790R, I790V, E791A, E791D, E791F, E791M, E791S, E791T, E791V, E791W, V792C, V792G, V792L, V792S, V792Y, D793C, D793F, D793H, D793K, D793N, D793Y, V794C, V794D, V794L, V794Q, V794T, V794W, T795P, T795Y, D796M, D796Q, D796S, D796T, A797H, A797K, N798G, N798I, N798P, N798Q, G799D, G799K, G799L, G799M, G799Q, G799Y, H800A, H800F, H800G, H800L, H800S, H800V, I801C, I801E, I801W, V802E, V802I, V802P, V802S, V802Y, P803A, P803F, P803G, P803K, P803S, P803Y, D804E, D804G, D804K, D804N, D804S, A805C, A805F, A805G, A805I, A805N, A805P, A806F, A806I, A806Q, N807F, N807Q, N807V, N807W, R808C, R808F, R808G, R808I, R808N, R808P, R808Q, V809A, V809C, V809L, V809M, V809P, T810L, T810P, T810Q, T810R, T810Y, F811L, F811Y, D812E, D812F, D812I, D812Q, V813F, V813H, V813T, V813W, V813Y, K814G, K814H, K814I, K814L, K814P, G815A, G815F, G815M, G815P, G815V, A816C, A816D, A816F, A816I, A816N, A816V, A816W, G817C, G817H, G817I, G817N, G817S, K818D, K818F, K818L, K818Q, K818R, K818S, K818V, K818W, K818Y, L819F, L819W, V820C, V820F, V820I, V820K, V820R, V820W, G821A, G821C, G821E, G821F, G821I, G821K, G821M, G821N, G821V, G821Y, V822A, V822D, 822E, V822T, D823E, N824A, N824C, N824G, N824Q, G825A, S826A, S826F, S826G, S826I, S826L, S826R, S826W, S827C, S827Q, P828C, P828G, P828I, P828L, P828Y, D829C, D829I, D829S, D829T, D829V, H830E, H830G, H830M, H830P, H830Q, H830R, H830V, D831A, D831F, D831G, D831I, D831M, D831P, D831R, D831V, S832E, S832F, S832G, S832L, S832M, S832P, S832R, S832V, S832W, Y833C, Y833D, Y833E, Y833I, Y833K, Y833N, Y833P, Y833V, Q834F, Q834G, Q834M, A835D, A835E, A835F, A835H, A835K, A835W, D836C, D836E, D836H, D836Q, D836R, D836S, D836T, D836V, D836W, D836Y, N837D, N837F, N837G, N837H, N837L, N837P, N837T, R838D, R838F, R838G, R838K, R838M, R838N, R838S, R838W, K839A, K839C, K839D, K839E, K839G, K839L, K839N, K839P, K839R, A840G, A840I, A840P, A840V, F841C, F841D, F841I, F841K, F841W, S842A, S842L, S842M, G843A, G843C, K844A, K844G, K844L, K844W, K844Y, V845N, V845W, L846G, L846I, L846M, L846S, A847L, A847T, I848M, V849A, V849L, V849S, V849T, Q850C, Q850G, Q850I, Q850L, Q850T, Q850V, Q850Y, S851C, S851D, S851E, S851L, S851T, T852D, T852G, T852L, K853N, K853P, K853Q, K853V, E854C, E854I, E854M, E854R, A855K, A855V, A855Y, E857P, E857V, I858D, I858E, I858F, I858G, I858K, I858M, I858P, I858Q, I858Y, T859V, V860T, V860Y, T861F, T861I, T861Q, T861V, T861W, A862C, A862P, A862V, K863F, K863I, K863L, K863N, K863W, A864E, A864H, A864K, A864L, D865A, D865G, G866H, G866R, L867W, S870E, S870M, S870R, S870T, T871P, V872C, V872G, K873G, K873Y, I874G, A875R, T877A, V879P, V879S, P880S, G881W, T882A, T882M, T882R, S883L, T884A, E885V, K886E, K886L, K886V, K886W, T887A, T887D, T887F, T887G, T887N, T887R, T887V, V888A, V888D, V888G, R889G, Y892D, Y892P, Y892R, Y893E, Y893G, S894D, S894G, R895M, N896M, Y897V, Y898T, V899G, K900E, K900G, T901G, T901Q, T901R, T901V, T901Y, G902A, G902D, G902F, G902L, G902P, G902Q, G902R, G902S, G902W, N903D, N903I, K904D, K904E, K904M, K904N, K904R, K904S, K904V, K904W, P905A, P905C, P905R, P905V, P905W, P905Y, I906A, I906D, I906N, I906S, I906T, I906W, I906Y, L907F, L907S, L907Y, P908C, P908D, P908G, P908I, P908L, P908M, P908T, S909E, S909F, S909G, S909W, S909Y, D910C, D910I, D910S, D910W, V911A, V911S, E912A, E912K, E912L, E912T, E912V, V913G, V913Q, V913R, V913W, R914A, R914E, R914F, R914I, R914K, R914V, R914W, R914Y, Y915A, Y915C, Y915G, Y915I, Y915M, Y915Q, Y915V, S916G, D917C, D917E, D917F, D917L, D917M, D917Q, D917S, G918E, G918H, G918T, G918V, G918W, T919D, T919K, T919Q, T919W, T919Y, S920C, S920E, S920M, S920P, S920R, S920V, S920W, D921C, D921P, D921Q, D921V, R922A, R922G, R922M, R922V, R922W, Q923A, Q923C, Q923E, Q923L, Q923M, Q923V, Q923W, N924A, N924L, N924P, N924Q, N924S, N924W, V925A, V925C, V925E, V925G, V925K, V925N, V925S, V925W, T926G, T926P, T926R, T926S, T926V, T926W, W927C, W927G, W927P, D928A, D928E, D928H, D928L, D928Q, A929C, A929P, A929V, V930A, V930E, V930I, V930K, V930M, V930T, S931G, S931P, S931R, D932F, D932R, D932S, D932T, D932V, D933I, D933R, D933S, Q934S, Q934V, I935A, I935C, I935D, I935E, I935L, I935P, I935V, I935W, A936I, A936L, A936Q, A936R, A936Y, K937G, K937I, K937M, K937P, K937Q, K937R, K937V, A938C, A938H, A938N, A938T, A938V, A938W, G939D, G939K, S940C, S940E, S940M, S940R, S940T, S940V, S940W, F941C, F941M, F941W, S942A, S942E, S942K, S942L, S942P, S942T, S942V, V943A, V943G, V943H, V943Q, V943R, A944D, A944G, A944H, A944P, A944R, A944V, G945E, G945P, G945T, T946A, T946E, T946G, T946L, T946P, T946V, T946W, V947G, V947H, V947L, V947M, V947P, V947R, V947T, A948C, A948I, A948R, A948W, G949A, G949F, G949V, Q950D, Q950G, Q950K, Q950M, Q950W, K951D, K951G, K951P, K951Q, K951S, K951W, K951Y, I952H, I952Q, S953F, S953M, S953N, S953R, S953W, V954D, V954L, V954Q, V954S, V954T, R955A, R955C, R955E, R955K, R955Q, R955W, V956A, V956D, V956G, V956H, V956I, V956M, V956Q, V956W, T957D, T957S, T957W, M958D, M958I, M958K, I959A, I959L, I959S, I959V, I959Y, D960G, D960H, D960L, D960P, D960S, D960W, E961D, E961F, E961K, E961P, E961S, E961T, I962A, I962C, I962D, I962G, I962K, I962N, I962Q, I962T, G963C, G963E, G963L, G963P, A964C, A964E, A964H, L965C, L965E, L965G, L965K, L965M, L965P, L965Q, L965S, L965V, L965Y, L966A, L966G, L966H, L966K, L966N, L966P, L966Q, L966S, L966T, L966V, N967C, N967D, N967I, N967L, N967M, N967P, N967S, N967T, N967V, Y968G, Y968L, Y968Q, Y968V, S969C, S969D, S969G, S969H, S969I, S969L, S969M, S969P, S969Q, S969Y, A970I, A970L, S971E, S971F, S971G, S971H, S971V, S971W, P973C, P973D, P973K, P973N, P973Q, P973R, P973V, P973W, P973Y, V974C, V974E, V974G, V974N, V974T, V974Y, G975D, G975F, G975K, G975L, G975Q, G975V, T976D, T976F, T976G, T976K, T976L, T976P, T976S, T976Y, P977A, P977C, P977K, P977R, P977T, P977Y, A978F, A978G, A978M, A978N, A978P, A978R, A978S, A978Y, V979G, V979N, V979R, V979Y, L980A, L980F, L980H, L980I, L980K, L980N, L980Q, L980T, L980Y, P981L, P981M, G982A, G982H, G982M, G982P, G982Q, G982W, R984P, R984S, P985E, P985F, P985H, P985K, P985L, P985W, A986C, A986E, A986F, A986I, A986K, A986L, A986M, A986N, A986S, A986W, V987A, V987C, V987F, V987I, V987K, V987L, V987Q, V987T, L988A, L988C, L988E, L988G, L988H, L988M, L988Q, L988R, L988S, L988V, L988Y, P989A, P989C, P989D, P989G, P989H, P989I, P989M, P989N, P989Q, P989W, D990F, D990P, D990S, D990W, G991C, G991F, G991H, G991K, G991P, G991Y, T992E, T992H, T992M, T992N, T992Y, V993D, V993G, V993N, V993S, T994I, T994S, T994V, S995E, S995L, S995R, S995V, A996Q, A996R, A996V, N997A, N997C, N997E, N997K, N997L, N997S, N997V, N997W, N997Y, F998M, F998W, A999F, A999G, A999L, A999M, A999R, A999S, V1000C, V1000L, V1000M, V1000N, V1000P, V1000W, D1001G, D1001K, D1001L, D1001M, D1001Q, D1001S, D1001T, D1001V, D1001Y, W1002A, W1002D, W1002E, W1002H, W1002N, W1002P, W1002Q, W1002S, T1003F, T1003G, T1003L, T1003N, T1003P, T1003R, T1003S, T1003W, T1003Y, K1004D, K1004E, K1004F, K1004G, K1004H, K1004M, K1004P, K1004R, K1004S, K1004V, P1005I, P1005N, P1005Q, P1005V, P1005Y, A1006C, A1006I, A1006N, A1006P, A1006S, A1006V, A1006W, A1006Y, D1007C, D1007L, D1007P, D1007V, T1008G, V1009G, V1009S, Y1010A, Y1010P, Y1010R, Y1010T, N1011A, N1011S, N1011T, N1011W, T1012E, T1012H, T1012I, T1012Q, T1012Y, A1013D, A1013K, A1013Q, A1013T, A1013V, G1014E, G1014I, G1014L, G1014M, G1014V, G1014W, G1014Y, T1015A, T1015F, T1015G, T1015V, V1016C, V1016D, V1016P, K1017E, K1017G, V1018I, V1018K, V1018L, V1018M, V1018R, V1018S, V1018W, T1021C, T1021E, T1021F, T1021G, T1021K, T1021L, T1021S, T1021V, A1022H, A1022L, A1022S, A1022Y, T1023D, T1023M, T1023Q, T1023R, V1024E, V1024G, V1024H, V1024K, V1024N, V1024R, V1024S, V1024W, G1026E, G1026H, G1026L, G1026R, G1026S, G1026V, G1026Y, K1027C, K1027N, K1027Q, K1027R, K1027V, E1028G, E1028S, E1028T, F1029I, F1029K, F1029L, F1029P, F1029V, F1029W, F1029Y, K1030D, K1030F, K1030H, K1030L, K1030M, K1030W, V1031H, V1031K, V1031Y, A1033G, A1033S, A1033V, T1034G, T1034H, T1034N, T1034W, I1035D, I1035G, I1035Q, R1036G, R1036L, R1036T, R1036Y, V1037C, V1037F, V1037P, V1037Q, Q1038A, Q1038D, Q1038K, R1039S, R1039V, S1040A, S1040M, S1040N, S1040R, S1040W, Q1041P, V1042N, T1043F, T1043G, T1043N, T1043R, I1044A, I1044L, G1045S, S1046I, S1046M, S1047D, V1048C, V1048F, V1048G, V1048I, V1048M, V1048Q, G1050L, G1050S, G1050V, N1051A, N1051E, N1051K, A1052C, A1052K, A1052M, A1052P, A1052R, L1053A, L1053W, R1054C, R1054L, R1054N, L1055R, L1055T, Q1057A, Q1057E, Q1057P, Q1057R, N1058R, N1058S, N1058V, N1058W, I1059W, P1060G, P1060N, P1060Q, P1060S, P1060T, A1061E, A1061G, A1061K, A1061W, D1062A, D1062F, D1062G, D1062I, D1062L, D1062M, D1062P, D1062S, K1063D, K1063M, Q1064C, Q1064M, Q1064R, Q1064T, Q1064V, S1065A, S1065C, S1065E, S1065G, S1065T, S1065W, D1066A, D1066G, D1066M, D1066V, D1066W, T1067G, T1067M, L1068C, L1068E, L1068P, L1068Q, L1068Y, D1069G, D1069K, D1069R, D1069W, A1070P, A1070T, I1071M, I1071R, I1071W, K1072E, K1072G, K1072P, K1072Q, K1072S, D1073F, D1073L, D1073M, D1073P, D1073W, G1074I, G1074L, G1074R, S1075C, S1075G, S1075I, S1075L, S1075V, T1076C, T1076E, T1076H, T1076Q, T1076S, T1077K, T1077L, T1077R, V1078D, V1078E, V1078L, V1078W, D1079G, D1079L, N1081D, N1081E, N1081G, T1082A, T1082C, T1082E, T1082F, T1082G, T1082K, T1082N, T1082S, G1083E, G1083F, G1083L, G1083P, G1083S, G1084C, G1084M, G1084V, G1084W, G1084Y, G1085A, G1085P, G1085R, G1085S, A1086H, A1086K, A1086Q, A1086R, A1086T, N1087A, N1087E, N1087I, N1087R, N1087V, N1087W, P1088D, P1088E, P1088G, P1088R, P1088W, S1089C, S1089E, S1089G, S1089K, S1089Q, S1089R, S1089V, A1090F, A1090G, A1090I, A1090K, W1091A, W1091E, W1091G, W1091H, W1091T, W1091V, W1091Y, T1092A, T1092E, T1092G, T1092K, T1092Q, T1092S, T1092V, N1093A, N1093G, N1093L, N1093P, N1093Q, N1093T, N1093V, W1094D, W1094E, W1094P, W1094R, W1094T, A1095P, A1095R, A1095T, A1095W, Y1096A, Y1096D, Y1096H, Y1096L, Y1096R, S1097D, S1097E, S1097K, S1097L, S1097T, S1097W, K1098D, K1098F, K1098G, K1098Q, K1098S, A1099C, A1099D, A1099F, A1099S, A1099V, A1099W, G1100D, G1100E, G1100H, G1100M, G1100N, G1100T, H1101K, H1101L, H1101Q, H1101R, H1101V, N1102E, N1102F, N1102H, N1102K, N1102L, N1102Q, N1102R, N1102T, T1103A, T1103E, T1103H, T1103S, T1103W, A1104I, A1104K, A1104R, E1105L, E1105S, I1106T, I1106V, T1107C, T1107M, T1107R, T1107S, F1108D, F1108K, F1108L, F1108T, F1108W, E1109A, E1109D, E1109L, E1109W, A1111G, A1111S, E1113D, E1113G, E1113P, E1113V, Q1114E, Q1114I, Q1114L, Q1114R, Q1114S, Q1114T, Q1114V, Q1115A, Q1115K, Q1115L, Q1115P, Q1115T, Q1115W, L1116D, L1116G, L1116H, L1116K, L1116V, L1116W, G1117E, G1117I, G1117M, G1117R, G1117S, G1117T, G1117V, G1117W, Q1118A, Q1118M, Q1118S, Q1118T, Q1118W, I1119D, I1119E, I1119G, I1119N, I1119S, V1120N, V1120S, V1120T, M1121G, M1121K, M1121N, M1121P, M1121S, M1121V, M1121Y, Y1122A, Y1122C, Y1122I, Y1122K, Y1122R, Y1122V, Y1122W, F1123E, F1123H, F1123R, F1123T, F1124E, F1124R, F1124V, F1124W, R1125D, R1125E, R1125F, R1125K, R1125T, R1125V, R1125W, D1126H, D1126K, D1126L, D1126R, S1127F, S1127I, S1127K, S1127M, S1127Q, S1127T, S1127W, N1128A, N1128C, N1128R, N1128S, N1128T, N1128W, A1129E, A1129L, A1129N, A1129Q, A1129R, A1129V, V1130A, V1130G, V1130P, V1130R, V1130S, R1131A, R1131N, R1131Q, R1131S, R1131W, F1132E, F1132K, F1132M, F1132P, F1132Q, F1132T, P1133D, P1133G, P1133L, P1133Q, P1133R, P1133V, D1134E, D1134G, D1134L, A1135E, A1135K, A1135L, A1135M, A1135S, A1135W, A1135Y, G1136A, G1136E, G1136P, G1136Q, G1136T, K1137A, K1137C, K1137G, K1137L, K1137P, K1137Q, K1137R, K1137S, K1137T, K1137V, T1138R, T1138Y, K1139A, K1139L, K1139R, K1139T, I1140A, I1140C, I1140G, I1140L, I1140M, I1140P, I1140R, I1140T, Q1141A, Q1141C, Q1141G, Q1141K, Q1141N, Q1141P, Q1141T, Q1141W, I1142E, I1142R, I1142S, I1142W, I1142Y, S1143G, A1144C, A1144D, A1144E, A1144N, A1144P, A1144S, A1144T, A1144V, A1144W, D1145E, D1145R, D1145T, G1146A, G1146C, G1146D, G1146K, G1146L, G1146R, G1146V, K1147A, K1147G, K1147T, K1147V, N1148H, N1148I, N1148K, N1148P, N1148Q, N1148R, N1148S, N1148T, N1148W, W1149C, W1149G, W1149I, W1149K, W1149N, W1149Q, W1149S, W1149T, W1149V, W1149Y, T1150G, T1150K, T1150P, D1151C, D1151G, D1151R, D1151T, D1151W, L1152A, L1152C, L1152E, L1152Q, L1152W, A1153E, A1153G, A1153K, A1153L, A1154C, A1154D, A1154E, A1154G, A1154R, A1154S, T1155E, T1155L, T1155Q, T1155R, T1157V, T1157W, I1158R, I1158S, I1158W, A1159C, A1159E, A1159I, A1159P, A1159R, A1159V, A1160K, A1160L, A1160Q, A1160S, Q1161A, Q1161P, Q1161S, E1162A, E1162C, E1162D, E1162F, E1162I, E1162N, E1162Q, E1162T, E1162W, E1162Y, E1165D, E1165H, E1165L, E1165M, E1165R, E1165S, E1165W, R1166D, R1166K, R1166Q, V1167A, V1167C, V1167L, V1167P, V1167R, K1168L, K1168Q, K1168R, K1168W, P1169M, P1169R, P1169S, Y1170E, Y1170K, Y1170M, Y1170Q, Y1170R, Y1170V, T1171A, T1171G, T1171M, T1171Q, T1171R, T1171S, Y1172D, Y1172E, Y1172H, Y1172I, Y1172K, Y1172L, Y1172S, Y1172V, D1173A, D1173E, D1173F, D1173G, D1173K, D1173L, D1173P, D1173R, D1173T, D1173W, F1174D, F1174P, F1174Q, F1174R, F1174S, F1174T, F1174V, F1174W, A1175G, A1175I, A1175N, A1175Q, A1175S, A1175V, A1175Y, V1177N, V1177P, V1177S, V1177T, G1178M, G1178Q, G1178S, G1178T, A1179L, A1179P, A1179Q, A1179W, T1180A, T1180G, T1180I, T1180L, T1180M, T1180Q, T1180S, T1180Y, F1181E, F1181L, F1181V, V1182M, K1183A, K1183E, K1183T, K1183V, V1184C, V1184E, V1184K, V1184L, V1184P, V1184Q, V1184Y, T1185Q, T1185V, V1186S, N1188V, N1188W, A1189C, A1189K, A1189T, A1189V, D1190R, D1190S, D1190T, D1190Y, T1191E, T1191L, T1192H, T1192P, T1193G, P1194A, P1194E, P1194G, P1194W, S1195G, V1197A, V1198E, C1199D, C1199T, A1200G, A1200V, A1200W, L1202A, L1202C, T1203E, T1203K, E1204G, E1204S, I1205V, K1208R, K1208V, K1208W, T1209A, T1209C, T1209E, T1209K, T1209N, T1209Q, T1209R, T1209W, A1210D, A1210E, A1210G, A1210K, A1210L, A1210Q, A1210R, A1210T, A1210W, T1211C, T1211D, T1211E, T1211G, T1211H, T1211K, T1211P, T1211Q, T1211R, T1211S, T1211V, K1213A, K1213D, K1213S, K1213T, K1213W, F1214A, F1214E, F1214K, F1214L, F1214P, F1214R, F1214S, F1214V, V1215D, V1215E, V1215K, V1215L, V1215Q, V1215S, V1215W, T1216A, T1216L, T1216P, T1216Q, T1216R, N1217A, N1217D, N1217E, N1217F, N1217P, N1217R, N1217S, N1217T, T1218A, T1218C, T1218E, T1218G, T1218Q, T1218S, T1218V, T1218W, S1219A, S1219E, S1219F, S1219I, S1219K, S1219R, S1219V, A1220C, A1220G, A1220L, A1220P, A1220R, A1220V, A1221D, A1221G, A1221K, A1221L, A1221R, A1221V, A1221W, L1222A, L1222C, L1222E, L1222F, L1222Q, L1222R, L1222V, L1222W, S1223C, S1223F, S1223G, S1223K, S1223L, S1223V, S1224A, S1224D, S1224G, S1224L, S1224M, S1224P, S1224R, S1224W, L1225C, L1225D, L1225E, L1225F, L1225G, L1225K, L1225P, L1225T, L1225V, L1225W, T1226A, T1226G, T1226M, T1226P, T1226R, T1226S, T1226V, T1226Y, V1227A, V1227C, V1227D, V1227E, V1227G, V1227L, V1227P, V1227Q, V1227S, N1228A, N1228D, N1228F, N1228K, N1228L, N1228T, G1229A, G1229C, G1229E, G1229Q, G1229S, G1229V, T1230F, T1230H, T1230I, T1230K, T1230L, T1230P, T1230R, T1230S, T1230W, K1231F, K1231G, K1231L, K1231M, K1231P, K1231S, K1231W, V1232E, V1232K, V1232Q, V1232R, V1232S, V1232T, V1232W, S1233P, S1233W, D1234G, D1234K, D1234R, D1234V, S1235D, S1235E, S1235G, S1235L, S1235P, S1235R, S1235W, S1235Y, V1236A, V1236C, V1236G, V1236I, V1236P, V1236Q, V1236R, L1237D, L1237E, L1237R, L1237V, L1237W, A1238D, A1238E, A1238K, A1238L, A1238N, A1238P, A1238R, A1238S, A1238T, A1239D, A1239P, A1239R, G1240D, G1240L, G1240N, G1240Q, G1240S, G1240T, G1240W, S1241D, S1241G, S1241I, S1241L, S1241M, S1241P, Y1242C, Y1242E, Y1242K, Y1242R, Y1242S, Y1242W, N1243C, N1243L, N1243M, N1243P, N1243Q, N1243S, N1243T, N1243V, N1243W, T1244A, T1244D, T1244E, T1244G, T1244L, T1244Q, T1244S, T1244V, T1244W, A1246F, A1246M, A1246N, A1246P, A1246Q, A1246R, A1246S, A1246T, I1247A, I1247G, I1247M, I1247Q, I1247R, I1247S, I1247T, I1247V, I1247W, I1248A, I1248G, I1248K, I1248L, I1248R, I1248S, I1248Y, A1249E, A1249G, A1249H, A1249I, A1249R, A1249T, A1249V, D1250I, D1250K, D1250S, D1250T, D1250W, D1250Y, V1251I, V1251T, V1251W, K1252D, K1252G, K1252V, K1252W, A1253P, A1253V, E1254F, E1254G, E1254H, E1254L, E1254R, E1254V, G1255H, G1255M, G1255S, G1255V, E1256G, E1256M, E1256N, E1256R, E1256V, E1256W, G1257F, G1257K, G1257L, G1257Q, G1257R, G1257W, N1258C, N1258G, N1258H, N1258K, N1258S, A1259K, A1259L, A1259W, V1261I, V1261L, V1261P, V1261Q, V1261R, V1261T, T1262A, T1262F, T1262M, T1262Q, T1262R, V1263E, V1263G, V1263Q, V1263R, V1263T, V1263W, L1264A, L1264E, L1264H, L1264R, L1264S, L1264Y, P1265C, P1265K, P1265L, P1265R, P1265S, P1265V, P1265W, A1266F, A1266L, A1266P, A1266S, A1266V, H1267A, H1267E, H1267F, N1269A, N1269E, N1269K, N1269R, N1269S, N1269T, N1269W, V1270D, V1270E, V1270G, V1270I, V1270L, V1270T, V1270W, I1271A, I1271H, I1271Q, R1272E, R1272F, R1272M, R1272P, R1272V, V1273R, I1274F, I1274M, I1274R, T1275A, T1275L, T1275W, E1276R, E1276W, S1277L, S1277T, S1277W, E1278Q, E1278R, D1279G, D1279I, D1279R, D1279T, D1279V, D1279W, H1280C, H1280E, H1280G, H1280V, H1280W, V1281F, V1281I, V1281S, V1281W, T1282D, T1282L, T1282V, R1283A, R1283D, R1283E, R1283P, R1283W, K1284G, T1285A, T1285E, T1285F, T1285G, T1285M, T1285R, T1285Y, F1286A, F1286E, F1286P, F1286R, F1286S, F1286T, T1287C, T1287K, T1287L, T1287M, T1287Q, T1287R, T1287S, T1287W, I1288A, I1288D, I1288F, I1288G, I1288K, N1289A, N1289Q, N1289T, L1290A, L1290G, L1290R, L1290V, L1290W, G1291K, G1291P, G1291V, G1291W, G1291Y, T1292G, T1292L, T1292Y, E1293G, E1293K, E1293L, E1293S, E1293V, Q1294E, Q1294L, Q1294P, Q1294W, E1295K, F1296A, F1296G, F1296I, P1297F, A1298Y, D1301I, E1302G, E1302R, E1302S, R1303S, D1304A or D1304V.

186. The method of the preceding embodiment wherein the substitutions are the substitutions listed in any one of embodiments 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144 or 146.

187. The method of any of the four preceding embodiments, wherein the variant has 1-50 substitutions, preferably 1-30, 1-20, 1-10 or 1-5, such as 1, 2, 3, 4, 5, 6, 7, 8, 9, 10, 11, 12, 13, 14, 15, 16, 17, 18, 19 or 20 substitutions, compared to the parent lactase.

188. The method of any of the five preceding embodiments, wherein the parent lactase is obtained from Bifidobacterium, preferably Bifidobacterium bifidum.

189. The method of any of the six preceding embodiments, wherein the parent lactase has an amino acid sequence which is at least 85%, 90%, or 95%, preferably at least 97%, 98%, or 99%, more preferably at least 99.5%, 99.7%, 99.8% or 99.9%, most preferably 100%, identical to SEQ ID NO: 1.

190. The method of any of the seven preceding embodiments, wherein the parent lactase has a length of 800-2000 amino acids, such as 1200-2000 amino acids, preferably 1200-1500 amino acids, more preferably 1250-1400 amino acids or 1250-1350 amino acids, even more preferably 1280-1320 amino acids, such as 1300-1320 amino acids or 1300-1310 amino acids, most preferably 1300-1305, such as 1302-1304 amino acids.

191. The method of any of the eight preceding embodiments, wherein the parent lactase comprises the same number of amino acid residues as the lactase of SEQ ID NO: 1.

192. The method of any of the nine preceding embodiments, wherein the variant has a decreased or an increased galactose inhibition, preferably measured as a galactose inhibition improvement factor (GI-IF) of at least 1.1, such as at least 1.2, at least 1.3, at least 1.4 or at least 1.5, or at most 0.9, such as at most 0.8, at most 0.7, at most 0.6 or at most 0.5, compared to the parent lactase.

193. The method of any of the ten preceding embodiments, wherein the variant has a decreased galactose inhibition, preferably measured as a galactose inhibition improvement factor (GI-IF) of at least 1.1, such as at least 1.2, at least 1.3, at least 1.4 or at least 1.5, compared to the parent lactase.

194. The method of any of the eleven preceding embodiments, wherein the variant has a decreased or an increased galactose inhibition, preferably measured as a galactose inhibition improvement factor (GI-IF) of at least 1.1, such as at least 1.2, at least 1.3, at least 1.4, at least 1.5, at least 1.6, at least 1.7, at least 1.8 or at least 1.9, or at most 0.9, such as at most 0.8, at most 0.7, at most 0.6, at most 0.5, at most 0.4, at most 0.3, at most 0.2 or at most 0.1, compared to the lactase of SEQ ID NO: 1.

195. The method of any of the twelve preceding embodiments, wherein the variant has a decreased galactose inhibition, preferably measured as a galactose inhibition improvement factor (GI-IF) of at least 1.1, such as at least 1.2, at least 1.3, at least 1.4, at least 1.5, at least 1.6, at least 1.7, at least 1.8 or at least 1.9, compared to the lactase of SEQ ID NO: 1.

196. The lactase variant of embodiment 97 having an amino acid sequence which is at least 99.4% identical to SEQ ID NO: 1.

197. The lactase variant of the preceding embodiment which has a length of 800-2000 amino acids, such as 1200-2000 amino acids, preferably 1200-1500 amino acids, more preferably 1250-1400 amino acids or 1250-1350 amino acids, even more preferably 1280-1320 amino acids, such as 1300-1320 amino acids or 1300-1310 amino acids, most preferably 1300-1305, such as 1302-1304 amino acids.

I98. The lactase variant of embodiment 101 having an amino acid sequence which is at least 99.4% identical to SEQ ID NO: 1.

199. The lactase variant of the preceding embodiment which has a length of 800-2000 amino acids, such as 1200-2000 amino acids, preferably 1200-1500 amino acids, more preferably 1250-1400 amino acids or 1250-1350 amino acids, even more preferably 1280-1320 amino acids, such as 1300-1320 amino acids or 1300-1310 amino acids, most preferably 1300-1305, such as 1302-1304 amino acids.

200. The lactase variant of embodiment 105 having an amino acid sequence which is at least 99.4% identical to SEQ ID NO: 1.

201. The lactase variant of the preceding embodiment which has a length of 800-2000 amino acids, such as 1200-2000 amino acids, preferably 1200-1500 amino acids, more preferably 1250-1400 amino acids or 1250-1350 amino acids, even more preferably 1280-1320 amino acids, such as 1300-1320 amino acids or 1300-1310 amino acids, most preferably 1300-1305, such as 1302-1304 amino acids.

202. The lactase variant of embodiment 109 having an amino acid sequence which is at least 99.4% identical to SEQ ID NO: 1.

203. The lactase variant of the preceding embodiment which has a length of 800-2000 amino acids, such as 1200-2000 amino acids, preferably 1200-1500 amino acids, more preferably 1250-1400 amino acids or 1250-1350 amino acids, even more preferably 1280-1320 amino acids, such as 1300-1320 amino acids or 1300-1310 amino acids, most preferably 1300-1305, such as 1302-1304 amino acids.

204. The lactase variant of embodiment 113 having an amino acid sequence which is at least 99.4% identical to SEQ ID NO: 1.

205. The lactase variant of the preceding embodiment which has a length of 800-2000 amino acids, such as 1200-2000 amino acids, preferably 1200-1500 amino acids, more preferably 1250-1400 amino acids or 1250-1350 amino acids, even more preferably 1280-1320 amino acids, such as 1300-1320 amino acids or 1300-1310 amino acids, most preferably 1300-1305, such as 1302-1304 amino acids.

206. The lactase variant of embodiment 117 having an amino acid sequence which is at least 99.4% identical to SEQ ID NO: 1.

207. The lactase variant of the preceding embodiment which has a length of 800-2000 amino acids, such as 1200-2000 amino acids, preferably 1200-1500 amino acids, more preferably 1250-1400 amino acids or 1250-1350 amino acids, even more preferably 1280-1320 amino acids, such as 1300-1320 amino acids or 1300-1310 amino acids, most preferably 1300-1305, such as 1302-1304 amino acids.

208. The lactase variant of embodiment 121 having an amino acid sequence which is at least 99.4% identical to SEQ ID NO: 1.

209. The lactase variant of the preceding embodiment which has a length of 800-2000 amino acids, such as 1200-2000 amino acids, preferably 1200-1500 amino acids, more preferably 1250-1400 amino acids or 1250-1350 amino acids, even more preferably 1280-1320 amino acids, such as 1300-1320 amino acids or 1300-1310 amino acids, most preferably 1300-1305, such as 1302-1304 amino acids.

210. The lactase variant of embodiment 125 having an amino acid sequence which is at least 99.4% identical to SEQ ID NO: 1.

211. The lactase variant of the preceding embodiment which has a length of 800-2000 amino acids, such as 1200-2000 amino acids, preferably 1200-1500 amino acids, more preferably 1250-1400 amino acids or 1250-1350 amino acids, even more preferably 1280-1320 amino acids, such as 1300-1320 amino acids or 1300-1310 amino acids, most preferably 1300-1305, such as 1302-1304 amino acids.

212. The lactase variant of embodiment 129 having an amino acid sequence which is at least 99.4% identical to SEQ ID NO: 1.

213. The lactase variant of the preceding embodiment which has a length of 800-2000 amino acids, such as 1200-2000 amino acids, preferably 1200-1500 amino acids, more preferably 1250-1400 amino acids or 1250-1350 amino acids, even more preferably 1280-1320 amino acids, such as 1300-1320 amino acids or 1300-1310 amino acids, most preferably 1300-1305, such as 1302-1304 amino acids.

214. The lactase variant of embodiment 133 having an amino acid sequence which is at least 99.4% identical to SEQ ID NO: 1.

215. The lactase variant of the preceding embodiment which has a length of 800-2000 amino acids, such as 1200-2000 amino acids, preferably 1200-1500 amino acids, more preferably 1250-1400 amino acids or 1250-1350 amino acids, even more preferably 1280-1320 amino acids, such as 1300-1320 amino acids or 1300-1310 amino acids, most preferably 1300-1305, such as 1302-1304 amino acids.

216. The lactase variant of embodiment 137 having an amino acid sequence which is at least 99.4% identical to SEQ ID NO: 1.

217. The lactase variant of the preceding embodiment which has a length of 800-2000 amino acids, such as 1200-2000 amino acids, preferably 1200-1500 amino acids, more preferably 1250-1400 amino acids or 1250-1350 amino acids, even more preferably 1280-1320 amino acids, such as 1300-1320 amino acids or 1300-1310 amino acids, most preferably 1300-1305, such as 1302-1304 amino acids.

218. The lactase variant of embodiment 141 having an amino acid sequence which is at least 99.4% identical to SEQ ID NO: 1.

219. The lactase variant of the preceding embodiment which has a length of 800-2000 amino acids, such as 1200-2000 amino acids, preferably 1200-1500 amino acids, more preferably 1250-1400 amino acids or 1250-1350 amino acids, even more preferably 1280-1320 amino acids, such as 1300-1320 amino acids or 1300-1310 amino acids, most preferably 1300-1305, such as 1302-1304 amino acids.

220. The lactase variant of embodiment 145 having an amino acid sequence which is at least 99.4% identical to SEQ ID NO: 1.

221. The lactase variant of the preceding embodiment which has a length of 800-2000 amino acids, such as 1200-2000 amino acids, preferably 1200-1500 amino acids, more preferably 1250-1400 amino acids or 1250-1350 amino acids, even more preferably 1280-1320 amino acids, such as 1300-1320 amino acids or 1300-1310 amino acids, most preferably 1300-1305, such as 1302-1304 amino acids.

### EXAMPLES

### Example 1: Site-Saturation Library Generation

A gene encoding the lactase of SEQ ID NO: 1* was cloned into a Bacillus subtilis expression cassette and transformed in a Bacillus subtilis expression host. Site-saturation libraries were generated by Mega PCR approach in each position in the gene of SEQ ID NO: 1 with NNS doping in the forward mutagenic primer.

Two PCR reactions were performed: 1) generation of C-terminal fragment with the flanking C-terminal reverse primer and the forward mutagenic primer 2) generation of Mega PCR product using the C-terminal fragment as the reverse mega-primer and the flanking N-terminal forward primer to give the full-length cassette. The Mega PCR product was then transformed into the Bacillus host, where site-specific homologous recombination in the Bacillus chromosome takes place. The polymerase used for the PCR reaction was Phusion Hot Start Flex DNA polymerase (New England Biolabs, Cat. No.: M0535L).

After overnight growth in TB (Terrific Broth) media with appropriate antibiotic in 96-well plates at 37° C. with 700 rpm, a second overnight culture was started in fresh TB media with appropriate antibiotic and incubated overnight at same conditions. This culture was diluted into single cells in 384-well plates in LB (Luria-Bertani) media and incubated overnight at 37° C.

Culture PCR was carried out on 384-well plates by initial heat lysis of cells, followed by PCR. The polymerase used for the culture PCR reaction was Phire Hot Start II DNA polymerase (ThermoScientific, Cat. No.: F126L).

The PCR products were sequence confirmed and the unique substitutions were collected and combined in 384-well plates and grown in LB media. The polymerase used for the PCR reaction was Phusion Hot Start Flex DNA polymerase (New England Biolabs, Cat. No.: M0535L).

The variants obtained in the resulting 384-well plates were confirmed by an additional sequencing, and confirmed variants were used in screening assays.

*Note regarding backbone: Some clones of our template backbone for the site saturation library (SEQ ID NO: 1) contain a G856E mutation, so all substitution variants we describe are in the backbone context of SEQ ID NO: 1 with either Gly or Glu at position 856. That mutation is far away from the active site of the enzyme, and our experimental data showed no significant difference for Gly or Glu at position 856 in any of the characteristics we tested in purified samples, such as specific activity (Km at pH 6.5:17.8 mM (G856) vs. 17.4 mM (E856); IF=0.98), galactose inhibition (Ki at pH 6.5:41.5 mM (G856) vs. 40.6 mM (E856); IF=0.98) or melting point (Tm at pH 6.5:54.7° C. (G856) vs. 54.4° C. (E856); IF=0.99). All characteristics are the same for the two backbones within the range of uncertainty of the measurements.

### Example 2: Galactose Inhibition Assay

The Bifidobacterium lactase SSL libraries, grown in Bacillus subtilis, were grown in microtiter plates (MTP) in TB+10% w/v Sucrose (80 μL/MTP well) for expression, for 3 days at 37° C. in 384-well lidded MTP's with agitation. The expression plates contained wild-type control expressed in B. subtilis, as well as non-inoculated control wells (used as the blanks in the calculation of relative activity below). The supernatants were diluted 8-fold in dilution buffer (MES (100 mM), pH 6.0), and the diluted enzyme solutions (10 μL) were transferred to two separate transparent 384-well plates containing 1) reference assay solution (40 μL, MES (100 mM), ONPG (10.66 mM), DMSO (140 mM), pH 6.0, in working concentrations) and 2) galactose assay solution (40 μL, D-(+)-Galactose (80 mM), MES (100 mM), ortho-Nitrophenyl-β-galactoside (ONPG, 10.66 mM), Dimethyl sulfoxide (DMSO, 140 mM), pH 6.0, in working concentrations). The assay plates were briefly agitated, followed by incubation at 30 min at 23° C. Stop solution (Na2CO3, 10% w/v in H2O) was added to the assay plates to stop the reaction. The endpoint absorbance was recorded at 405 nm of the two assay plates.

The adjusted absorbances from the assay plates were used to calculate the ratio of activity (RA), defined as RA=(Abs405 Galactose sample-Abs405 Galactose Blank)/(Abs405 Reference sample-Abs405 Reference Blank).

The samples that displayed an absorbance at 405 nm below 0.2 in either the galactose or reference condition were removed.

The galactose inhibition assay improvement factor (GI-IF) of a given variant is calculated relative to the RA of the wild type enzyme of SEQ ID NO: 1, which is normalized to have an IF=1.0, per expression plate or per entire experiment if the given plate did not contain any wild types with assay data that passed the QC filter.

QC (quality control) filter: As quality control, from all variants constructed, we retained only those variants with an unstressed reference absorbance (Abs405 Reference sample) bigger than 0.2 and a ratio of activity (RA) bigger than 0.0 and less than 1.0.

A GI-IF above 1 means that the variant has a decreased galactose inhibition compared to the wild type enzyme, whereas a GI-IF below 1 means that the variant has an increased galactose inhibition.

Variants showing decreased galactose inhibition (i.e. GI-IF at least 1.5, or at least 1.4, or at least 1.3, or at least 1.2, or at least 1.1, but more than 1.0) are beneficial in application, since they lead to higher lactase performance. A decreased galactose inhibition will allow the enzyme to retain more activity at higher levels of galactose and thus allow for degradation of lactose down to a lower final lactose level. Moreover, variants showing increased galactose inhibition (i.e. GI-IF at most 0.5, or at most 0.6, or at most 0.7, or at most 0.8, or at most 0.9, but less than 1.0) are also beneficial in application, since they allow to fine-tune the galactose inhibition propensity of the lactase. An increased galactose inhibition may reduce or stop the activity of the enzyme when a certain decreased level of lactose has been obtained which may be advantageous in some commercial applications.

The variants that displayed a GI-IF of at least 1.1 or at most 0.9 are shown below.

TABLE 1
Variants having a GI-IF of at least 1.1

| Position | Mutation | GI-IF |
|---|---|---|
| 1 | V1D | 1.23 |
| 1 | V1F | 1.25 |
| 1 | V1H | 1.31 |
| 1 | V1K | 1.11 |
| 1 | V1L | 1.35 |
| 1 | V1R | 1.12 |
| 2 | E2D | 1.36 |
| 2 | E2G | 1.17 |
| 2 | E2Q | 1.25 |
| 2 | E2V | 1.45 |
| 3 | D3A | 1.13 |
| 3 | D3I | 1.41 |
| 3 | D3N | 1.12 |
| 3 | D3V | 1.48 |
| 4 | A4H | 1.19 |
| 5 | T5A | 1.12 |
| 5 | T5S | 1.22 |
| 6 | R6G | 1.15 |
| 6 | R6H | 1.13 |
| 6 | R6L | 1.14 |
| 6 | R6W | 1.13 |
| 7 | S7L | 1.11 |
| 7 | S7N | 1.39 |
| 7 | S7P | 2.24 |
| 7 | S7T | 1.12 |
| 9 | S9G | 1.15 |
| 9 | S9H | 1.21 |
| 9 | S9W | 1.10 |
| 10 | T10K | 1.15 |
| 10 | T10L | 1.21 |
| 10 | T10S | 1.21 |
| 12 | Q12V | 1.17 |
| 13 | M13C | 1.34 |
| 13 | M13D | 1.29 |
| 13 | M13E | 1.10 |
| 13 | M13H | 1.14 |
| 13 | M13K | 1.20 |
| 13 | M13R | 1.48 |
| 13 | M13W | 1.11 |
| 14 | S14G | 1.19 |
| 14 | S14H | 1.16 |
| 14 | S14T | 1.16 |
| 14 | S14V | 1.36 |
| 14 | S14Y | 1.14 |
| 15 | S15C | 1.45 |
| 15 | S15F | 1.13 |
| 15 | S15I | 2.18 |
| 15 | S15K | 1.58 |
| 15 | S15L | 1.11 |
| 15 | S15P | 1.25 |
| 15 | S15R | 1.27 |
| 15 | S15T | 1.38 |
| 15 | S15V | 1.33 |
| 15 | S15W | 1.19 |
| 16 | T16C | 1.57 |
| 16 | T16I | 1.20 |
| 17 | P17A | 1.15 |
| 17 | P17I | 1.37 |
| 17 | P17S | 1.14 |
| 19 | V19A | 1.10 |
| 19 | V19F | 1.15 |
| 19 | V19I | 1.14 |
| 19 | V19K | 1.25 |
| 19 | V19L | 1.16 |
| 19 | V19N | 1.15 |
| 19 | V19S | 1.18 |
| 20 | V20C | 1.35 |
| 20 | V20F | 1.19 |
| 20 | V20G | 1.23 |
| 20 | V20I | 1.12 |
| 20 | V20L | 1.21 |
| 20 | V20M | 1.36 |
| 20 | V20P | 1.89 |
| 20 | V20Q | 1.28 |
| 20 | V20R | 1.26 |
| 20 | V20T | 1.10 |
| 21 | Y21A | 1.14 |
| 21 | Y21C | 1.40 |
| 21 | Y21D | 1.16 |
| 21 | Y21G | 1.12 |
| 21 | Y21H | 1.12 |
| 21 | Y21M | 1.19 |
| 21 | Y21P | 1.32 |
| 21 | Y21R | 1.43 |
| 21 | Y21T | 1.45 |
| 22 | S22A | 1.18 |
| 22 | S22N | 1.22 |
| 22 | S22R | 1.12 |
| 22 | S22T | 1.21 |
| 22 | S22W | 1.36 |
| 23 | S23A | 1.17 |
| 24 | A24F | 1.45 |
| 24 | A24L | 1.36 |
| 24 | A24R | 1.21 |
| 24 | A24T | 1.93 |
| 24 | A24W | 1.39 |
| 25 | V25D | 1.29 |
| 25 | V25E | 1.12 |
| 25 | V25F | 1.13 |
| 25 | V25G | 1.16 |
| 25 | V25M | 1.23 |
| 25 | V25Q | 1.33 |
| 25 | V25S | 1.21 |
| 25 | V25T | 1.18 |
| 25 | V25W | 1.12 |
| 26 | D26C | 1.47 |
| 26 | D26I | 1.25 |
| 26 | D26L | 1.24 |
| 26 | D26M | 1.13 |
| 26 | D26V | 1.12 |
| 27 | S27A | 1.76 |
| 27 | S27C | 1.36 |
| 27 | S27G | 1.25 |
| 27 | S27H | 1.34 |
| 27 | S27P | 1.11 |
| 27 | S27Y | 1.26 |
| 28 | K28C | 1.52 |
| 28 | K28G | 1.10 |
| 28 | K28I | 1.11 |
| 28 | K28L | 1.27 |
| 28 | K28R | 1.19 |
| 28 | K28S | 1.15 |
| 28 | K28V | 1.25 |
| 28 | K28W | 1.18 |
| 29 | Q29F | 1.11 |
| 29 | Q29G | 1.21 |
| 29 | Q29L | 1.52 |
| 29 | Q29M | 1.45 |
| 29 | Q29R | 1.41 |
| 29 | Q29S | 1.12 |
| 30 | N30M | 1.21 |
| 30 | N30V | 1.26 |
| 30 | N30W | 1.11 |
| 30 | N30Y | 1.16 |
| 31 | R31E | 1.70 |
| 31 | R31M | 1.25 |
| 31 | R31V | 1.41 |
| 32 | T32Q | 1.19 |
| 32 | T32S | 1.59 |
| 33 | S33K | 1.12 |
| 34 | D34F | 1.12 |
| 34 | D34G | 1.27 |
| 34 | D34H | 1.25 |
| 35 | F35A | 1.12 |
| 35 | F35C | 1.34 |
| 35 | F35G | 1.12 |
| 35 | F35V | 1.15 |
| 36 | D36Q | 1.28 |
| 37 | A37N | 1.12 |
| 38 | N38S | 1.26 |
| 39 | W39G | 1.37 |
| 39 | W39S | 1.23 |
| 40 | K40C | 1.40 |
| 40 | K40F | 1.42 |
| 40 | K40G | 1.23 |
| 40 | K40I | 1.46 |
| 40 | K40M | 1.18 |
| 40 | K40P | 1.11 |
| 40 | K40W | 1.27 |
| 41 | F41A | 1.35 |
| 41 | F41C | 1.28 |
| 42 | M42T | 1.40 |
| 43 | L43A | 1.21 |
| 43 | L43G | 1.13 |
| 43 | L43I | 1.21 |
| 43 | L43S | 1.33 |
| 44 | S44M | 1.17 |
| 45 | D45L | 1.16 |
| 45 | D45P | 1.68 |
| 45 | D45V | 1.11 |
| 47 | V47K | 1.43 |
| 47 | V47R | 1.68 |
| 49 | A49C | 1.13 |
| 49 | A49D | 1.57 |
| 49 | A49H | 1.73 |
| 49 | A49T | 1.46 |
| 51 | D51G | 1.23 |
| 51 | D51I | 1.61 |
| 51 | D51K | 1.42 |
| 51 | D51M | 1.19 |
| 53 | A53G | 1.20 |
| 55 | D55C | 1.33 |
| 55 | D55F | 1.28 |
| 55 | D55G | 1.27 |
| 55 | D55H | 1.53 |
| 55 | D55M | 1.42 |
| 55 | D55N | 1.14 |
| 55 | D55P | 1.36 |
| 55 | D55S | 1.25 |
| 55 | D55V | 1.13 |
| 57 | S57A | 1.28 |
| 57 | S57C | 1.69 |
| 57 | S57E | 2.31 |
| 57 | S57G | 1.79 |
| 58 | A58I | 1.17 |
| 58 | A58N | 1.15 |
| 58 | A58Q | 1.32 |
| 58 | A58R | 1.14 |
| 58 | A58T | 1.51 |
| 59 | W59D | 1.10 |
| 59 | W59I | 1.30 |
| 59 | W59K | 1.39 |
| 59 | W59L | 1.11 |
| 59 | W59N | 1.11 |
| 59 | W59P | 1.86 |
| 59 | W59V | 1.22 |
| 60 | Q60E | 1.11 |
| 60 | Q60F | 1.16 |
| 60 | Q60G | 1.17 |
| 60 | Q60L | 1.12 |
| 60 | Q60M | 1.24 |
| 60 | Q60R | 1.34 |
| 60 | Q60S | 1.34 |
| 60 | Q60V | 1.15 |
| 60 | Q60Y | 1.22 |
| 61 | Q61P | 1.22 |
| 62 | V62G | 1.18 |
| 62 | V62N | 1.47 |
| 62 | V62T | 1.39 |
| 62 | V62W | 1.34 |
| 63 | D63G | 1.29 |
| 63 | D63L | 1.18 |
| 63 | D63S | 1.11 |
| 64 | L64E | 1.48 |
| 64 | L64G | 1.28 |
| 66 | H66R | 1.32 |
| 66 | H66T | 1.20 |
| 70 | I70H | 1.18 |
| 70 | I70K | 1.30 |
| 70 | I70P | 1.11 |
| 70 | I70R | 1.14 |
| 71 | T71H | 1.12 |
| 71 | T71L | 1.12 |
| 71 | T71P | 1.11 |
| 71 | T71R | 1.18 |
| 71 | T71S | 1.18 |
| 73 | K73A | 1.15 |
| 73 | K73D | 1.49 |
| 73 | K73G | 1.11 |
| 73 | K73Q | 1.12 |
| 73 | K73V | 1.13 |
| 74 | Y74G | 1.20 |
| 74 | Y74K | 1.22 |
| 75 | S75G | 1.53 |
| 75 | S75L | 1.43 |
| 75 | S75Q | 1.29 |
| 75 | S75R | 1.42 |
| 75 | S75V | 1.40 |
| 76 | Q76G | 1.51 |
| 76 | Q76I | 1.40 |
| 76 | Q76P | 1.11 |
| 77 | S77C | 1.20 |
| 77 | S77D | 1.36 |
| 77 | S77E | 1.41 |
| 77 | S77G | 1.35 |
| 77 | S77H | 1.73 |
| 77 | S77I | 1.15 |
| 77 | S77K | 1.51 |
| 77 | S77L | 1.25 |
| 77 | S77M | 1.23 |
| 77 | S77R | 1.35 |
| 77 | S77T | 1.24 |
| 77 | S77V | 1.29 |
| 77 | S77W | 1.17 |
| 78 | N78C | 1.43 |
| 78 | N78F | 1.17 |
| 78 | N78S | 1.21 |
| 79 | E79H | 1.44 |
| 79 | E79S | 1.45 |
| 79 | E79T | 1.45 |
| 83 | A83E | 1.16 |
| 83 | A83T | 1.33 |
| 85 | L85A | 1.33 |
| 85 | L85C | 1.19 |
| 85 | L85D | 1.19 |
| 85 | L85F | 2.06 |
| 85 | L85N | 1.34 |
| 85 | L85S | 1.47 |
| 85 | L85V | 1.42 |
| 85 | L85W | 1.53 |
| 86 | P86E | 1.11 |
| 86 | P86G | 1.24 |
| 86 | P86Q | 1.99 |
| 86 | P86R | 1.24 |
| 86 | P86V | 1.29 |
| 86 | P86W | 1.21 |
| 86 | P86Y | 1.22 |
| 87 | G87D | 1.74 |
| 87 | G87N | 1.21 |
| 88 | G88A | 1.11 |
| 88 | G88F | 1.74 |
| 88 | G88M | 1.11 |
| 89 | T89C | 1.12 |
| 89 | T89L | 1.11 |
| 89 | T89M | 1.29 |
| 89 | T89N | 1.17 |
| 89 | T89W | 1.15 |
| 89 | T89Y | 1.28 |
| 90 | G90A | 1.24 |
| 90 | G90D | 1.11 |
| 90 | G90L | 1.22 |
| 90 | G90S | 1.21 |
| 90 | G90T | 1.61 |
| 91 | W91E | 1.29 |
| 91 | W91L | 1.27 |
| 91 | W91P | 1.35 |
| 91 | W91Q | 1.22 |
| 91 | W91Y | 1.43 |
| 92 | Y92F | 1.21 |
| 92 | Y92H | 1.46 |
| 92 | Y92I | 1.31 |
| 92 | Y92M | 1.67 |
| 92 | Y92N | 1.29 |
| 92 | Y92S | 1.70 |
| 92 | Y92T | 1.30 |
| 92 | Y92V | 1.70 |
| 92 | Y92W | 1.16 |
| 93 | R93A | 2.08 |
| 93 | R93D | 1.42 |
| 93 | R93F | 1.94 |
| 93 | R93H | 1.55 |
| 93 | R93I | 1.36 |
| 93 | R93L | 1.95 |
| 93 | R93N | 1.50 |
| 93 | R93T | 1.83 |
| 93 | R93V | 1.76 |
| 93 | R93Y | 1.93 |
| 94 | K94A | 1.33 |
| 94 | K94C | 1.20 |
| 94 | K94G | 1.21 |
| 94 | K94R | 1.14 |
| 94 | K94S | 1.60 |
| 94 | K94T | 1.31 |
| 94 | K94V | 1.26 |
| 95 | S95A | 1.28 |
| 95 | S95C | 1.49 |
| 95 | S95D | 1.35 |
| 95 | S95E | 1.15 |
| 95 | S95G | 1.50 |
| 95 | S95I | 1.59 |
| 95 | S95L | 1.14 |
| 95 | S95Q | 1.28 |
| 95 | S95R | 1.26 |
| 96 | F96A | 1.77 |
| 96 | F96C | 1.61 |
| 96 | F96I | 1.49 |
| 96 | F96K | 1.35 |
| 96 | F96L | 1.13 |
| 96 | F96M | 1.35 |
| 96 | F96P | 1.31 |
| 96 | F96V | 1.66 |
| 96 | F96W | 1.25 |
| 97 | T97F | 1.42 |
| 97 | T97S | 1.18 |
| 97 | T97V | 1.29 |
| 98 | I98C | 1.33 |
| 98 | I98H | 1.51 |
| 98 | I98S | 1.17 |
| 98 | I98W | 1.22 |
| 100 | R100T | 1.40 |
| 101 | D101A | 1.46 |
| 101 | D101P | 1.71 |
| 101 | D101V | 1.20 |
| 102 | L102A | 1.16 |
| 102 | L102G | 1.11 |
| 102 | L102M | 1.18 |
| 102 | L102P | 1.51 |
| 102 | L102S | 1.15 |
| 104 | G104C | 1.12 |
| 105 | K105D | 1.20 |
| 105 | K105Q | 1.12 |
| 105 | K105R | 1.14 |
| 105 | K105W | 1.14 |
| 105 | K105Y | 1.13 |
| 106 | R106K | 1.19 |
| 106 | R106V | 1.24 |
| 106 | R106W | 1.39 |
| 107 | I107A | 1.19 |
| 107 | I107F | 1.24 |
| 107 | I107S | 1.31 |
| 108 | A108E | 1.35 |
| 108 | A108V | 1.16 |
| 109 | I109M | 1.50 |
| 109 | I109T | 1.16 |
| 110 | N110A | 1.36 |
| 110 | N110F | 1.15 |
| 110 | N110S | 1.35 |
| 110 | N110T | 1.42 |
| 110 | N110V | 1.30 |
| 110 | N110W | 1.33 |
| 111 | F111A | 1.48 |
| 111 | F111C | 1.14 |
| 111 | F111L | 1.48 |
| 111 | F111Q | 1.28 |
| 111 | F111V | 1.16 |
| 112 | D112A | 1.13 |
| 114 | V114F | 1.23 |
| 114 | V114R | 1.50 |
| 115 | Y115E | 1.43 |
| 116 | M116A | 1.19 |
| 116 | M116C | 1.39 |
| 116 | M116D | 1.14 |
| 116 | M116W | 1.17 |
| 117 | N117K | 1.13 |
| 118 | A118P | 1.35 |
| 119 | T119G | 1.12 |
| 120 | V120A | 1.58 |
| 120 | V120K | 1.32 |
| 121 | W121C | 1.14 |
| 121 | W121D | 1.28 |
| 121 | W121R | 1.69 |
| 121 | W121T | 1.16 |
| 121 | W121V | 1.54 |
| 124 | G124M | 1.22 |
| 125 | V125D | 1.18 |
| 126 | K126E | 1.12 |
| 126 | K126V | 1.16 |
| 129 | T129V | 1.20 |
| 130 | H130A | 1.43 |
| 130 | H130C | 1.20 |
| 130 | H130S | 1.22 |
| 130 | H130T | 1.50 |
| 131 | P131K | 1.17 |
| 132 | Y132E | 1.42 |
| 137 | F137C | 1.22 |
| 137 | F137D | 1.15 |
| 137 | F137L | 1.50 |
| 137 | F137P | 1.16 |
| 138 | S138A | 1.26 |
| 138 | S138D | 1.11 |
| 138 | S138H | 1.14 |
| 138 | S138M | 1.14 |
| 140 | D140V | 1.20 |
| 141 | L141G | 1.27 |
| 142 | T142S | 1.31 |
| 148 | G148K | 1.13 |
| 149 | G149E | 1.25 |
| 149 | G149I | 1.14 |
| 149 | G149M | 1.56 |
| 149 | G149Y | 1.20 |
| 150 | E150A | 1.15 |
| 150 | E150C | 1.22 |
| 150 | E150G | 1.21 |
| 150 | E150L | 1.44 |
| 150 | E150N | 1.10 |
| 150 | E150R | 1.26 |
| 151 | N151L | 1.13 |
| 154 | V154E | 1.18 |
| 154 | V154I | 1.25 |
| 154 | V154K | 1.33 |
| 154 | V154L | 1.40 |
| 154 | V154M | 1.14 |
| 157 | V157A | 1.19 |
| 157 | V157G | 1.17 |
| 157 | V157P | 1.44 |
| 157 | V157Q | 1.19 |
| 157 | V157S | 1.39 |
| 158 | E158H | 1.58 |
| 158 | E158K | 1.19 |
| 158 | E158Q | 1.12 |
| 158 | E158V | 1.30 |
| 159 | N159D | 1.12 |
| 159 | N159H | 1.21 |
| 159 | N159T | 1.52 |
| 160 | R160G | 1.12 |
| 161 | L161E | 1.15 |
| 161 | L161K | 1.41 |
| 161 | L161M | 1.39 |
| 161 | L161S | 1.75 |
| 161 | L161W | 1.57 |
| 162 | P162F | 1.14 |
| 162 | P162G | 1.12 |
| 162 | P162N | 2.00 |
| 162 | P162T | 1.64 |
| 162 | P162W | 1.48 |
| 164 | S164A | 1.33 |
| 165 | R165H | 1.88 |
| 166 | W166S | 1.83 |
| 167 | Y167A | 1.97 |
| 167 | Y167C | 1.29 |
| 169 | G169A | 1.15 |
| 169 | G169S | 1.11 |
| 170 | S170Q | 1.72 |
| 171 | G171C | 1.22 |
| 171 | G171F | 1.16 |
| 172 | I172G | 1.12 |
| 172 | I172K | 1.32 |
| 172 | I172P | 1.55 |
| 172 | I172Q | 1.19 |
| 173 | Y173A | 1.19 |
| 173 | Y173H | 1.30 |
| 173 | Y173M | 1.25 |
| 173 | Y173P | 1.19 |
| 173 | Y173S | 1.17 |
| 174 | R174E | 2.27 |
| 174 | R174K | 1.22 |
| 175 | D175Y | 1.11 |
| 176 | V176E | 1.44 |
| 176 | V176K | 1.41 |
| 176 | V176T | 1.27 |
| 177 | T177C | 1.14 |
| 177 | T177E | 1.20 |
| 177 | T177K | 1.45 |
| 178 | L178I | 1.54 |
| 178 | L178Q | 1.51 |
| 178 | L178W | 1.25 |
| 179 | T179A | 1.26 |
| 179 | T179C | 1.44 |
| 179 | T179D | 1.26 |
| 179 | T179H | 1.19 |
| 179 | T179I | 1.39 |
| 179 | T179K | 1.26 |
| 179 | T179L | 1.15 |
| 179 | T179N | 1.38 |
| 179 | T179P | 1.29 |
| 179 | T179S | 1.28 |
| 180 | V180A | 1.28 |
| 180 | V180C | 1.19 |
| 180 | V180D | 1.33 |
| 180 | V180E | 1.15 |
| 180 | V180G | 1.48 |
| 180 | V180M | 1.19 |
| 181 | T181A | 1.37 |
| 181 | T181F | 1.22 |
| 183 | G183W | 1.25 |
| 184 | V184F | 1.29 |
| 184 | V184H | 1.16 |
| 184 | V184P | 1.22 |
| 184 | V184Q | 1.20 |
| 184 | V184R | 1.31 |
| 184 | V184S | 1.29 |
| 184 | V184W | 1.24 |
| 185 | H185G | 1.43 |
| 185 | H185L | 1.79 |
| 185 | H185R | 1.12 |
| 186 | V186A | 1.15 |
| 186 | V186E | 1.26 |
| 186 | V186G | 1.16 |
| 186 | V186N | 1.26 |
| 187 | G187A | 1.10 |
| 187 | G187D | 1.40 |
| 187 | G187H | 1.25 |
| 188 | N188R | 1.23 |
| 188 | N188S | 1.16 |
| 188 | N188V | 1.56 |
| 188 | N188W | 1.12 |
| 189 | N189A | 1.16 |
| 190 | G190F | 1.33 |
| 190 | G190V | 1.18 |
| 191 | V191T | 1.12 |
| 191 | V191Y | 1.12 |
| 193 | I193N | 1.27 |
| 193 | I193Q | 1.26 |
| 193 | I193T | 1.15 |
| 193 | I193V | 1.10 |
| 194 | K194I | 1.38 |
| 195 | T195M | 1.19 |
| 195 | T195W | 1.25 |
| 196 | P196A | 1.21 |
| 196 | P196I | 1.20 |
| 196 | P196M | 1.15 |
| 196 | P196S | 1.12 |
| 196 | P196W | 1.14 |
| 197 | S197C | 1.45 |
| 197 | S197E | 1.25 |
| 197 | S197L | 1.25 |
| 198 | L198I | 1.17 |
| 199 | A199E | 1.27 |
| 199 | A199P | 1.32 |
| 199 | A199T | 1.10 |
| 201 | Q201E | 1.33 |
| 201 | Q201K | 1.19 |
| 202 | N202A | 1.17 |
| 202 | N202M | 1.11 |
| 202 | N202Q | 1.30 |
| 202 | N202S | 1.40 |
| 202 | N202W | 1.30 |
| 203 | G203Q | 1.16 |
| 204 | G204R | 1.16 |
| 205 | N205W | 1.11 |
| 206 | V206C | 1.19 |
| 207 | T207L | 1.15 |
| 213 | K213A | 1.19 |
| 215 | A215D | 1.21 |
| 216 | N216D | 1.12 |
| 217 | D217F | 1.20 |
| 217 | D217G | 1.14 |
| 217 | D217L | 2.29 |
| 217 | D217M | 1.77 |
| 218 | T218D | 1.11 |
| 218 | T218G | 1.61 |
| 218 | T218H | 1.18 |
| 219 | K219A | 1.13 |
| 220 | A220G | 1.70 |
| 221 | A221C | 1.10 |
| 221 | A221D | 1.15 |
| 222 | A222R | 2.01 |
| 223 | N223S | 1.67 |
| 224 | I224G | 1.12 |
| 225 | T225L | 1.14 |
| 226 | L226Q | 1.28 |
| 228 | Q228N | 1.25 |
| 228 | Q228R | 1.40 |
| 231 | F231L | 1.20 |
| 232 | P232T | 1.19 |
| 234 | G234V | 1.22 |
| 235 | G235K | 1.21 |
| 236 | K236M | 1.66 |
| 236 | K236P | 1.28 |
| 237 | T237D | 2.25 |
| 238 | D238K | 1.13 |
| 240 | A240Q | 1.11 |
| 242 | G242K | 1.34 |
| 242 | G242L | 1.20 |
| 242 | G242M | 1.21 |
| 242 | G242P | 1.20 |
| 243 | T243I | 1.21 |
| 247 | A247K | 1.22 |
| 248 | S248F | 1.16 |
| 251 | I251F | 1.19 |
| 251 | I251W | 1.28 |
| 251 | I251Y | 1.52 |
| 252 | A252H | 1.15 |
| 252 | A252P | 1.19 |
| 252 | A252W | 1.43 |
| 252 | A252Y | 1.71 |
| 257 | A257D | 1.30 |
| 257 | A257G | 1.12 |
| 257 | A257N | 1.28 |
| 257 | A257V | 1.25 |
| 260 | T260V | 1.14 |
| 261 | S261A | 1.11 |
| 261 | S261H | 1.37 |
| 261 | S261Y | 1.14 |
| 262 | T262E | 1.13 |
| 262 | T262F | 1.16 |
| 262 | T262P | 1.15 |
| 263 | I263A | 1.16 |
| 264 | T264L | 1.10 |
| 266 | A266D | 1.38 |
| 267 | S267V | 1.35 |
| 268 | P268F | 1.23 |
| 268 | P268R | 2.22 |
| 270 | L270N | 1.20 |
| 271 | W271T | 1.16 |
| 272 | S272T | 1.36 |
| 272 | S272W | 1.56 |
| 273 | I273W | 1.11 |
| 274 | K274Q | 1.16 |
| 275 | N275M | 1.15 |
| 277 | N277F | 1.28 |
| 280 | T280D | 1.11 |
| 281 | V281Q | 1.37 |
| 282 | R282W | 1.20 |
| 283 | T283M | 1.10 |
| 285 | V285H | 1.20 |
| 286 | L286W | 1.25 |
| 291 | V291H | 1.18 |
| 291 | V291L | 1.20 |
| 291 | V291P | 1.51 |
| 291 | V291S | 1.16 |
| 292 | L292A | 1.12 |
| 292 | L292D | 1.28 |
| 292 | L292E | 1.20 |
| 292 | L292Q | 1.14 |
| 292 | L292S | 1.17 |
| 292 | L292V | 1.45 |
| 293 | D293C | 1.47 |
| 293 | D293I | 1.23 |
| 293 | D293W | 1.33 |
| 294 | T294C | 1.19 |
| 294 | T294M | 1.13 |
| 294 | T294Q | 1.22 |
| 294 | T294S | 1.22 |
| 295 | Y295F | 1.31 |
| 295 | Y295G | 1.13 |
| 295 | Y295L | 1.12 |
| 295 | Y295S | 1.16 |
| 296 | D296F | 1.48 |
| 296 | D296H | 1.21 |
| 296 | D296K | 1.34 |
| 297 | T297I | 1.23 |
| 297 | T297S | 1.19 |
| 298 | E298I | 1.60 |
| 298 | E298L | 1.22 |
| 298 | E298M | 1.16 |
| 298 | E298R | 1.40 |
| 298 | E298T | 1.12 |
| 299 | Y299F | 1.11 |
| 299 | Y299S | 1.10 |
| 301 | F301C | 1.75 |
| 302 | R302I | 1.23 |
| 302 | R302K | 1.11 |
| 302 | R302M | 1.16 |
| 302 | R302T | 1.11 |
| 303 | W303A | 1.63 |
| 303 | W303C | 1.33 |
| 303 | W303D | 1.33 |
| 303 | W303F | 1.22 |
| 303 | W303T | 1.19 |
| 304 | T304D | 1.32 |
| 304 | T304E | 1.23 |
| 304 | T304I | 1.24 |
| 304 | T304K | 1.31 |
| 304 | T304P | 3.20 |
| 304 | T304S | 1.19 |
| 304 | T304W | 1.15 |
| 305 | G305E | 1.12 |
| 305 | G305L | 1.21 |
| 305 | G305M | 1.13 |
| 305 | G305N | 1.47 |
| 305 | G305P | 1.44 |
| 305 | G305T | 1.21 |
| 307 | D307A | 1.24 |
| 307 | D307E | 1.22 |
| 307 | D307F | 1.18 |
| 307 | D307S | 1.21 |
| 307 | D307V | 1.12 |
| 307 | D307W | 1.24 |
| 308 | A308G | 1.10 |
| 308 | A308I | 1.12 |
| 309 | T309C | 1.37 |
| 309 | T309D | 1.49 |
| 309 | T309E | 1.38 |
| 309 | T309I | 1.28 |
| 309 | T309K | 1.19 |
| 309 | T309V | 1.25 |
| 310 | S310A | 1.21 |
| 310 | S310C | 1.17 |
| 310 | S310G | 1.20 |
| 310 | S310H | 1.33 |
| 310 | S310I | 1.19 |
| 310 | S310L | 1.30 |
| 310 | S310M | 1.22 |
| 310 | S310N | 1.34 |
| 310 | S310R | 1.13 |
| 310 | S310V | 1.30 |
| 311 | G311F | 1.11 |
| 311 | G311L | 1.17 |
| 312 | F312H | 1.48 |
| 312 | F312L | 1.16 |
| 312 | F312V | 1.17 |
| 314 | L314A | 1.31 |
| 314 | L314V | 1.39 |
| 314 | L314Y | 1.12 |
| 315 | N315S | 1.36 |
| 316 | G316N | 1.12 |
| 316 | G316R | 1.20 |
| 316 | G316W | 1.16 |
| 316 | G316Y | 1.16 |
| 317 | E317V | 1.11 |
| 317 | E317W | 1.20 |
| 317 | E317Y | 1.19 |
| 318 | K318C | 1.25 |
| 318 | K318E | 1.16 |
| 318 | K318F | 1.15 |
| 318 | K318H | 1.15 |
| 318 | K318M | 1.18 |
| 318 | K318N | 1.33 |
| 318 | K318P | 1.12 |
| 318 | K318Q | 1.13 |
| 320 | K320E | 1.26 |
| 320 | K320I | 1.21 |
| 320 | K320V | 1.42 |
| 320 | K320W | 1.49 |
| 321 | L321H | 1.19 |
| 321 | L321M | 1.39 |
| 321 | L321Y | 1.14 |
| 322 | K322C | 1.19 |
| 322 | K322I | 1.34 |
| 324 | V324E | 1.17 |
| 324 | V324G | 1.16 |
| 324 | V324T | 1.31 |
| 325 | S325G | 1.48 |
| 326 | M326T | 1.57 |
| 326 | M326V | 1.11 |
| 328 | H328C | 1.42 |
| 328 | H328D | 1.11 |
| 328 | H328F | 1.32 |
| 328 | H328G | 1.33 |
| 328 | H328I | 1.12 |
| 328 | H328L | 1.23 |
| 328 | H328M | 1.42 |
| 328 | H328R | 1.27 |
| 328 | H328T | 1.40 |
| 331 | G331V | 1.39 |
| 335 | A335C | 1.21 |
| 335 | A335G | 1.38 |
| 335 | A335L | 1.31 |
| 337 | A337D | 1.19 |
| 337 | A337E | 1.20 |
| 337 | A337G | 1.49 |
| 337 | A337L | 1.89 |
| 338 | N338P | 1.26 |
| 338 | N338R | 1.13 |
| 340 | R340A | 1.45 |
| 340 | R340L | 1.35 |
| 341 | A341M | 1.54 |
| 342 | I342A | 1.10 |
| 342 | I342G | 1.33 |
| 342 | I342P | 1.21 |
| 343 | E343A | 1.19 |
| 343 | E343N | 1.40 |
| 343 | E343R | 1.17 |
| 343 | E343T | 1.28 |
| 343 | E343Y | 1.37 |
| 344 | R344G | 1.37 |
| 344 | R344Y | 1.15 |
| 345 | Q345F | 1.25 |
| 345 | Q345N | 1.28 |
| 345 | Q345S | 1.54 |
| 346 | V346A | 1.39 |
| 346 | V346I | 1.22 |
| 346 | V346S | 1.23 |
| 347 | E347A | 1.33 |
| 347 | E347D | 1.17 |
| 347 | E347I | 1.24 |
| 348 | I348A | 1.12 |
| 348 | I348D | 1.52 |
| 348 | I348G | 1.13 |
| 348 | I348M | 1.37 |
| 348 | I348Q | 1.29 |
| 348 | I348R | 1.26 |
| 349 | L349A | 1.21 |
| 349 | L349E | 1.11 |
| 349 | L349S | 1.24 |
| 349 | L349V | 1.15 |
| 350 | Q350G | 1.43 |
| 350 | Q350N | 1.29 |
| 350 | Q350W | 1.16 |
| 351 | K351R | 1.15 |
| 351 | K351W | 1.17 |
| 352 | M352L | 1.14 |
| 352 | M352T | 1.37 |
| 353 | G353K | 1.40 |
| 354 | V354E | 1.22 |
| 354 | V354G | 1.18 |
| 354 | V354S | 1.28 |
| 354 | V354W | 1.29 |
| 355 | N355H | 1.10 |
| 357 | I357T | 1.39 |
| 359 | T359E | 1.53 |
| 359 | T359L | 1.35 |
| 360 | T360V | 1.47 |
| 362 | N362S | 1.42 |
| 362 | N362T | 2.65 |
| 363 | P363A | 1.38 |
| 363 | P363D | 1.30 |
| 363 | P363I | 1.51 |
| 363 | P363L | 1.49 |
| 363 | P363M | 1.43 |
| 363 | P363S | 1.20 |
| 363 | P363V | 1.21 |
| 364 | A364C | 1.28 |
| 364 | A364E | 1.22 |
| 364 | A364F | 1.24 |
| 364 | A364G | 1.22 |
| 364 | A364I | 2.12 |
| 364 | A364M | 1.58 |
| 364 | A364P | 1.49 |
| 364 | A364V | 1.27 |
| 364 | A364W | 1.13 |
| 365 | A365C | 1.41 |
| 365 | A365I | 1.39 |
| 365 | A365P | 1.36 |
| 365 | A365V | 1.33 |
| 365 | A365W | 1.21 |
| 366 | K366D | 1.29 |
| 366 | K366I | 1.17 |
| 366 | K366L | 1.33 |
| 366 | K366M | 1.35 |
| 366 | K366P | 1.21 |
| 366 | K366S | 1.39 |
| 366 | K366V | 1.22 |
| 367 | A367I | 1.18 |
| 367 | A367N | 1.17 |
| 367 | A367Q | 1.26 |
| 368 | L368A | 1.34 |
| 368 | L368Q | 1.40 |
| 368 | L368S | 1.83 |
| 368 | L368V | 1.15 |
| 368 | L368Y | 1.21 |
| 369 | I369A | 1.22 |
| 369 | I369D | 1.22 |
| 369 | I369E | 1.35 |
| 369 | I369G | 1.48 |
| 369 | I369K | 4.08 |
| 369 | I369M | 1.22 |
| 369 | I369V | 1.39 |
| 369 | I369W | 1.56 |
| 370 | D370L | 1.58 |
| 370 | D370Q | 1.24 |
| 370 | D370R | 1.19 |
| 370 | D370T | 1.19 |
| 371 | V371F | 1.32 |
| 371 | V371G | 1.11 |
| 371 | V371I | 1.16 |
| 371 | V371L | 1.21 |
| 371 | V371Q | 1.33 |
| 372 | C372P | 1.31 |
| 373 | N373G | 1.60 |
| 373 | N373L | 1.49 |
| 373 | N373R | 1.19 |
| 374 | E374L | 1.12 |
| 375 | K375D | 1.30 |
| 375 | K375I | 1.12 |
| 375 | K375N | 1.14 |
| 375 | K375Q | 1.11 |
| 375 | K375S | 1.23 |
| 376 | G376A | 1.47 |
| 376 | G376S | 1.87 |
| 377 | V377A | 1.22 |
| 377 | V377M | 1.44 |
| 377 | V377T | 1.30 |
| 378 | L378I | 1.13 |
| 378 | L378P | 1.19 |
| 378 | L378Y | 1.14 |
| 379 | V379A | 1.46 |
| 379 | V379C | 1.15 |
| 379 | V379M | 1.19 |
| 379 | V379N | 1.36 |
| 380 | V380P | 1.45 |
| 380 | V380S | 1.17 |
| 381 | E381A | 1.54 |
| 381 | E381C | 1.17 |
| 381 | E381G | 1.30 |
| 381 | E381Q | 1.31 |
| 381 | E381T | 1.71 |
| 383 | V383A | 1.33 |
| 386 | M386G | 1.61 |
| 386 | M386N | 1.57 |
| 386 | M386S | 2.15 |
| 386 | M386V | 1.25 |
| 387 | W387H | 1.36 |
| 388 | N388E | 1.22 |
| 388 | N388L | 1.48 |
| 389 | R389A | 1.45 |
| 389 | R389C | 1.29 |
| 389 | R389E | 1.61 |
| 389 | R389K | 1.16 |
| 389 | R389M | 1.16 |
| 389 | R389Q | 2.11 |
| 389 | R389S | 2.92 |
| 389 | R389T | 1.53 |
| 389 | R389V | 1.17 |
| 390 | S390C | 1.81 |
| 390 | S390D | 1.64 |
| 390 | S390H | 1.17 |
| 390 | S390P | 1.60 |
| 390 | S390T | 1.70 |
| 393 | G393E | 1.34 |
| 393 | G393R | 1.80 |
| 393 | G393V | 6.87 |
| 395 | T395A | 1.20 |
| 395 | T395C | 1.58 |
| 395 | T395F | 1.20 |
| 396 | E396K | 1.80 |
| 396 | E396L | 2.25 |
| 396 | E396V | 2.70 |
| 396 | E396W | 1.78 |
| 398 | Y398M | 1.35 |
| 399 | G399S | 1.58 |
| 400 | K400A | 1.52 |
| 400 | K400C | 1.50 |
| 400 | K400M | 1.17 |
| 400 | K400N | 1.64 |
| 400 | K400T | 1.23 |
| 400 | K400V | 1.51 |
| 401 | W401F | 1.53 |
| 401 | W401K | 2.38 |
| 402 | F402W | 1.71 |
| 402 | F402Y | 1.88 |
| 403 | G403A | 2.67 |
| 403 | G403D | 1.20 |
| 403 | G403H | 1.17 |
| 403 | G403K | 1.70 |
| 403 | G403Q | 1.32 |
| 403 | G403T | 1.19 |
| 403 | G403V | 1.62 |
| 403 | G403Y | 1.28 |
| 404 | Q404F | 1.67 |
| 404 | Q404L | 1.70 |
| 404 | Q404M | 2.15 |
| 404 | Q404P | 1.98 |
| 404 | Q404S | 1.23 |
| 404 | Q404V | 2.92 |
| 405 | A405C | 1.84 |
| 405 | A405E | 1.50 |
| 405 | A405K | 1.33 |
| 405 | A405R | 1.58 |
| 405 | A405T | 2.14 |
| 405 | A405V | 1.17 |
| 406 | I406C | 1.23 |
| 406 | I406D | 1.67 |
| 406 | I406N | 1.63 |
| 407 | A407C | 1.39 |
| 407 | A407G | 1.33 |
| 407 | A407Q | 1.30 |
| 410 | N410R | 1.25 |
| 410 | N410Y | 1.32 |
| 411 | A411E | 1.50 |
| 411 | A411R | 1.24 |
| 411 | A411S | 1.22 |
| 412 | V412S | 1.20 |
| 414 | G414M | 1.40 |
| 414 | G414N | 1.32 |
| 414 | G414T | 1.14 |
| 415 | G415A | 1.13 |
| 415 | G415Q | 1.24 |
| 416 | D416M | 1.19 |
| 417 | K417F | 1.53 |
| 417 | K417R | 1.31 |
| 422 | A422P | 1.36 |
| 423 | K423L | 1.18 |
| 423 | K423R | 1.10 |
| 426 | L426C | 1.25 |
| 427 | T427D | 1.45 |
| 427 | T427F | 1.33 |
| 427 | T427Q | 2.11 |
| 427 | T427S | 1.62 |
| 427 | T427W | 1.25 |
| 429 | T429D | 1.82 |
| 429 | T429P | 1.12 |
| 430 | I430L | 1.28 |
| 430 | I430M | 1.20 |
| 431 | N431L | 1.11 |
| 431 | N431M | 1.51 |
| 432 | R432F | 1.17 |
| 432 | R432G | 1.15 |
| 432 | R432Q | 1.24 |
| 432 | R432Y | 1.21 |
| 433 | D433C | 1.17 |
| 433 | D433G | 1.33 |
| 433 | D433I | 1.31 |
| 433 | D433P | 1.50 |
| 433 | D433Q | 1.33 |
| 433 | D433W | 1.69 |
| 434 | R434N | 1.86 |
| 434 | R434T | 1.12 |
| 434 | R434V | 1.74 |
| 435 | N435E | 1.38 |
| 435 | N435F | 1.20 |
| 435 | N435H | 1.17 |
| 435 | N435L | 1.50 |
| 435 | N435V | 1.23 |
| 436 | A436C | 1.19 |
| 436 | A436G | 1.22 |
| 436 | A436L | 1.52 |
| 436 | A436M | 1.12 |
| 437 | P437K | 1.16 |
| 437 | P437L | 1.17 |
| 437 | P437W | 1.11 |
| 438 | S438G | 1.15 |
| 439 | V439C | 1.52 |
| 439 | V439K | 1.52 |
| 440 | I440S | 1.13 |
| 440 | I440T | 1.14 |
| 440 | I440V | 1.10 |
| 441 | M441R | 1.12 |
| 441 | M441T | 1.17 |
| 442 | W442E | 1.41 |
| 442 | W442G | 1.51 |
| 443 | S443C | 1.50 |
| 443 | S443G | 1.31 |
| 443 | S443M | 1.23 |
| 443 | S443Q | 1.36 |
| 444 | L444E | 1.33 |
| 444 | L444F | 1.25 |
| 444 | L444V | 1.76 |
| 446 | N446D | 1.37 |
| 448 | M448C | 1.50 |
| 448 | M448E | 1.14 |
| 448 | M448L | 3.81 |
| 449 | M449D | 1.86 |
| 449 | M449E | 1.24 |
| 449 | M449F | 1.11 |
| 449 | M449T | 1.58 |
| 449 | M449V | 1.13 |
| 449 | M449W | 1.81 |
| 449 | M449Y | 2.48 |
| 450 | E450F | 2.52 |
| 450 | E450S | 2.73 |
| 450 | E450T | 2.36 |
| 450 | E450V | 3.02 |
| 450 | E450W | 1.98 |
| 451 | G451C | 2.60 |
| 451 | G451F | 2.67 |
| 451 | G451L | 3.39 |
| 451 | G451P | 2.77 |
| 451 | G451V | 1.57 |
| 451 | G451W | 1.85 |
| 452 | I452K | 1.77 |
| 452 | I452L | 1.28 |
| 452 | I452Q | 1.25 |
| 452 | I452S | 1.35 |
| 452 | I452V | 1.32 |
| 453 | S453C | 1.11 |
| 453 | S453G | 1.15 |
| 453 | S453H | 1.25 |
| 453 | S453L | 1.25 |
| 453 | S453M | 1.18 |
| 453 | S453N | 1.23 |
| 453 | S453P | 1.13 |
| 453 | S453Q | 1.25 |
| 453 | S453R | 1.13 |
| 453 | S453V | 1.19 |
| 454 | G454L | 1.49 |
| 454 | G454W | 1.27 |
| 455 | S455E | 1.21 |
| 455 | S455M | 1.24 |
| 455 | S455P | 1.61 |
| 455 | S455R | 1.63 |
| 455 | S455V | 1.20 |
| 456 | V456D | 1.56 |
| 456 | V456E | 1.47 |
| 456 | V456F | 1.62 |
| 456 | V456K | 1.42 |
| 457 | S457H | 1.33 |
| 457 | S457Q | 1.24 |
| 457 | S457T | 1.49 |
| 457 | S457V | 1.54 |
| 458 | G458A | 1.52 |
| 458 | G458D | 1.15 |
| 458 | G458P | 1.47 |
| 458 | G458S | 1.38 |
| 459 | F459C | 1.27 |
| 459 | F459R | 2.04 |
| 460 | P460M | 1.15 |
| 460 | P460Y | 1.21 |
| 461 | A461G | 1.21 |
| 461 | A461M | 1.38 |
| 461 | A461S | 1.19 |
| 462 | T462E | 1.32 |
| 462 | T462L | 1.30 |
| 464 | A464W | 1.26 |
| 465 | K465C | 1.12 |
| 465 | K465G | 1.11 |
| 465 | K465L | 1.30 |
| 465 | K465Q | 1.13 |
| 465 | K465V | 1.34 |
| 465 | K465Y | 1.56 |
| 466 | L466G | 1.23 |
| 466 | L466P | 1.74 |
| 467 | V467T | 1.20 |
| 468 | A468D | 2.95 |
| 469 | W469L | 1.15 |
| 469 | W469R | 1.17 |
| 471 | K471F | 1.36 |
| 471 | K471Q | 1.22 |
| 471 | K471Y | 1.11 |
| 472 | A472G | 2.00 |
| 472 | A472Y | 2.65 |
| 473 | A473M | 1.10 |
| 473 | A473P | 1.16 |
| 474 | D474C | 1.16 |
| 474 | D474E | 1.10 |
| 474 | D474M | 1.17 |
| 474 | D474W | 1.28 |
| 475 | S475E | 1.44 |
| 475 | S475T | 1.16 |
| 476 | T476L | 1.13 |
| 476 | T476S | 1.14 |
| 477 | R477A | 1.12 |
| 477 | R477G | 1.18 |
| 477 | R477L | 1.49 |
| 478 | P478A | 1.13 |
| 478 | P478D | 1.18 |
| 478 | P478V | 1.22 |
| 479 | M479G | 2.01 |
| 480 | T480Q | 1.14 |
| 485 | K485E | 1.49 |
| 485 | K485R | 1.35 |
| 487 | K487A | 1.27 |
| 487 | K487C | 1.20 |
| 487 | K487F | 1.54 |
| 487 | K487S | 1.37 |
| 487 | K487W | 1.37 |
| 488 | A488C | 1.25 |
| 488 | A488L | 1.11 |
| 488 | A488V | 1.18 |
| 493 | S493E | 1.21 |
| 493 | S493G | 1.37 |
| 493 | S493H | 1.23 |
| 493 | S493L | 1.32 |
| 493 | S493Q | 1.45 |
| 494 | N494A | 1.13 |
| 494 | N494I | 1.13 |
| 494 | N494M | 1.21 |
| 494 | N494R | 1.40 |
| 494 | N494V | 1.25 |
| 495 | T495K | 1.10 |
| 497 | G497D | 1.39 |
| 499 | N499T | 1.50 |
| 505 | G505D | 2.06 |
| 505 | G505H | 1.26 |
| 505 | G505L | 1.27 |
| 505 | G505R | 1.38 |
| 505 | G505S | 1.93 |
| 505 | G505V | 1.13 |
| 506 | V506C | 1.75 |
| 506 | V506E | 2.03 |
| 506 | V506R | 1.75 |
| 507 | V507F | 1.12 |
| 507 | V507P | 1.18 |
| 507 | V507R | 1.69 |
| 508 | G508C | 2.24 |
| 508 | G508E | 2.53 |
| 509 | T509A | 1.37 |
| 509 | T509D | 1.17 |
| 509 | T509E | 1.12 |
| 509 | T509I | 1.22 |
| 509 | T509K | 1.86 |
| 509 | T509M | 1.54 |
| 509 | T509Q | 1.14 |
| 509 | T509S | 1.14 |
| 509 | T509V | 1.29 |
| 509 | T509Y | 1.21 |
| 510 | N510A | 2.45 |
| 510 | N510F | 1.62 |
| 510 | N510I | 2.74 |
| 511 | Y511A | 2.86 |
| 512 | S512C | 1.26 |
| 512 | S512E | 1.19 |
| 512 | S512F | 1.52 |
| 512 | S512I | 1.25 |
| 512 | S512M | 1.29 |
| 512 | S512Q | 1.35 |
| 512 | S512T | 1.63 |
| 512 | S512V | 1.31 |
| 512 | S512Y | 1.46 |
| 513 | D513G | 1.18 |
| 513 | D513P | 1.91 |
| 513 | D513R | 1.66 |
| 515 | A515E | 2.73 |
| 515 | A515F | 1.13 |
| 515 | A515G | 1.22 |
| 515 | A515P | 1.31 |
| 515 | A515S | 1.17 |
| 516 | N516C | 1.55 |
| 516 | N516E | 1.39 |
| 516 | N516G | 1.22 |
| 516 | N516I | 1.22 |
| 516 | N516M | 1.17 |
| 516 | N516Q | 1.40 |
| 516 | N516S | 1.10 |
| 516 | N516T | 1.23 |
| 516 | N516V | 1.33 |
| 517 | Y517G | 1.98 |
| 517 | Y517I | 1.15 |
| 518 | D518Y | 1.54 |
| 519 | K519C | 1.42 |
| 519 | K519E | 1.25 |
| 519 | K519F | 1.10 |
| 519 | K519G | 1.23 |
| 519 | K519I | 1.45 |
| 519 | K519L | 1.18 |
| 519 | K519M | 1.11 |
| 519 | K519N | 1.19 |
| 519 | K519Q | 1.26 |
| 519 | K519S | 1.16 |
| 519 | K519T | 1.16 |
| 520 | I520A | 1.25 |
| 520 | I520C | 1.30 |
| 520 | I520G | 1.45 |
| 520 | I520H | 1.49 |
| 520 | I520M | 1.34 |
| 520 | I520N | 1.20 |
| 520 | I520Q | 1.26 |
| 520 | I520S | 1.45 |
| 520 | I520T | 1.36 |
| 520 | I520W | 1.20 |
| 520 | I520Y | 1.18 |
| 521 | R521A | 1.20 |
| 521 | R521C | 1.16 |
| 521 | R521K | 1.14 |
| 521 | R521Q | 1.12 |
| 521 | R521V | 2.64 |
| 523 | T523A | 1.14 |
| 523 | T523M | 1.19 |
| 524 | H524R | 1.80 |
| 524 | H524V | 1.54 |
| 525 | P525R | 1.13 |
| 526 | S526G | 2.24 |
| 527 | W527A | 1.36 |
| 527 | W527E | 1.22 |
| 527 | W527G | 1.24 |
| 527 | W527H | 1.38 |
| 527 | W527N | 2.54 |
| 527 | W527R | 1.58 |
| 527 | W527S | 1.43 |
| 528 | A528E | 1.41 |
| 528 | A528G | 1.15 |
| 528 | A528L | 1.11 |
| 529 | I529G | 1.27 |
| 535 | A535E | 1.16 |
| 535 | A535G | 1.20 |
| 535 | A535I | 1.21 |
| 537 | A537M | 1.35 |
| 537 | A537P | 1.59 |
| 537 | A537S | 1.23 |
| 538 | I538H | 1.16 |
| 538 | I538M | 1.29 |
| 540 | S540E | 1.55 |
| 545 | N545G | 1.12 |
| 547 | T547K | 1.19 |
| 549 | G549D | 1.39 |
| 549 | G549P | 1.40 |
| 549 | G549W | 1.71 |
| 550 | G550Q | 1.19 |
| 550 | G550R | 1.17 |
| 553 | S553P | 1.20 |
| 553 | S553R | 1.17 |
| 555 | D555E | 1.18 |
| 555 | D555P | 1.19 |
| 556 | K556C | 1.12 |
| 556 | K556R | 1.23 |
| 558 | L558E | 1.31 |
| 558 | L558H | 1.23 |
| 558 | L558P | 1.77 |
| 559 | T559G | 1.11 |
| 559 | T559Q | 1.29 |
| 559 | T559V | 1.10 |
| 559 | T559Y | 1.41 |
| 560 | S560P | 1.15 |
| 560 | S560V | 1.18 |
| 564 | S564A | 1.25 |
| 564 | S564C | 1.56 |
| 564 | S564F | 1.85 |
| 564 | S564W | 1.36 |
| 565 | A565C | 1.29 |
| 565 | A565D | 1.32 |
| 565 | A565E | 1.46 |
| 565 | A565F | 1.24 |
| 565 | A565G | 1.27 |
| 565 | A565I | 1.57 |
| 565 | A565L | 1.39 |
| 565 | A565M | 1.19 |
| 565 | A565R | 1.52 |
| 565 | A565S | 1.24 |
| 565 | A565T | 1.35 |
| 565 | A565V | 1.12 |
| 565 | A565W | 1.20 |
| 567 | G567N | 1.11 |
| 567 | G567Q | 2.29 |
| 567 | G567V | 1.78 |
| 570 | A570G | 1.40 |
| 570 | A570L | 1.12 |
| 572 | A572W | 1.27 |
| 574 | S574V | 1.18 |
| 575 | A575D | 1.37 |
| 577 | Y577R | 1.14 |
| 578 | D578T | 1.18 |
| 580 | V580D | 1.20 |
| 581 | Q581T | 1.13 |
| 581 | Q581Y | 1.27 |
| 582 | R582A | 1.23 |
| 582 | R582G | 1.26 |
| 582 | R582I | 1.18 |
| 582 | R582L | 1.88 |
| 583 | D583V | 1.13 |
| 583 | D583W | 1.67 |
| 584 | F584I | 1.16 |
| 584 | F584W | 1.21 |
| 586 | A586D | 1.24 |
| 588 | T588P | 1.11 |
| 589 | Y589I | 1.26 |
| 589 | Y589Q | 1.24 |
| 591 | W591F | 1.39 |
| 592 | T592L | 1.42 |
| 593 | G593C | 1.11 |
| 594 | F594C | 1.14 |
| 597 | L597D | 1.29 |
| 597 | L597T | 1.10 |
| 598 | G598N | 1.36 |
| 600 | P600A | 1.51 |
| 600 | P600E | 2.15 |
| 600 | P600G | 1.49 |
| 600 | P600S | 2.01 |
| 604 | N604E | 1.25 |
| 604 | N604S | 1.11 |
| 605 | G605R | 1.20 |
| 607 | G607Q | 1.44 |
| 607 | G607S | 1.26 |
| 608 | S608E | 1.14 |
| 608 | S608I | 1.12 |
| 608 | S608N | 1.19 |
| 608 | S608Q | 1.19 |
| 608 | S608T | 1.21 |
| 609 | G609A | 1.28 |
| 609 | G609S | 1.18 |
| 610 | A610T | 1.25 |
| 612 | G612V | 1.10 |
| 613 | S613A | 1.15 |
| 614 | W614A | 1.86 |
| 615 | P615V | 1.11 |
| 619 | N619I | 1.31 |
| 619 | N619L | 1.43 |
| 619 | N619S | 1.10 |
| 621 | Y621W | 1.12 |
| 625 | V625A | 1.12 |
| 625 | V625E | 1.28 |
| 625 | V625F | 1.16 |
| 625 | V625M | 2.18 |
| 625 | V625Y | 2.15 |
| 627 | T627K | 1.39 |
| 627 | T627Q | 1.38 |
| 628 | A628C | 1.11 |
| 628 | A628D | 1.32 |
| 628 | A628N | 1.27 |
| 629 | G629T | 1.21 |
| 630 | F630C | 1.39 |
| 630 | F630Y | 1.39 |
| 631 | P631D | 1.36 |
| 631 | P631Y | 1.22 |
| 632 | K632C | 1.17 |
| 632 | K632D | 1.23 |
| 632 | K632G | 1.27 |
| 633 | D633G | 1.15 |
| 634 | T634F | 1.14 |
| 634 | T634S | 1.15 |
| 634 | T634V | 1.45 |
| 636 | Y636K | 1.19 |
| 637 | F637C | 1.17 |
| 637 | F637G | 1.23 |
| 637 | F637I | 1.23 |
| 637 | F637S | 1.21 |
| 637 | F637T | 1.29 |
| 637 | F637V | 1.26 |
| 639 | Q639R | 1.73 |
| 643 | N643R | 1.13 |
| 644 | D644Y | 1.21 |
| 645 | D645S | 1.21 |
| 645 | D645V | 1.14 |
| 646 | V646C | 1.17 |
| 646 | V646L | 1.16 |
| 646 | V646N | 1.17 |
| 646 | V646R | 1.21 |
| 646 | V646S | 1.12 |
| 647 | H647G | 1.24 |
| 647 | H647V | 1.18 |
| 648 | T648C | 1.27 |
| 649 | L649V | 1.10 |
| 650 | H650F | 1.12 |
| 651 | I651T | 1.24 |
| 651 | I651V | 1.16 |
| 652 | L652C | 1.25 |
| 652 | L652D | 1.17 |
| 652 | L652V | 1.14 |
| 652 | L652W | 1.20 |
| 653 | P653Q | 1.23 |
| 654 | A654D | 1.13 |
| 654 | A654R | 1.23 |
| 655 | W655F | 1.15 |
| 655 | W655R | 1.62 |
| 656 | N656K | 1.16 |
| 657 | E657K | 1.12 |
| 657 | E657R | 1.30 |
| 657 | E657V | 1.16 |
| 661 | A661E | 1.75 |
| 661 | A661G | 1.18 |
| 661 | A661H | 1.23 |
| 661 | A661K | 1.38 |
| 661 | A661L | 1.19 |
| 661 | A661M | 1.26 |
| 661 | A661Q | 1.36 |
| 661 | A661W | 1.20 |
| 662 | K662H | 1.14 |
| 662 | K662S | 1.26 |
| 662 | K662V | 1.27 |
| 662 | K662W | 1.16 |
| 662 | K662Y | 1.26 |
| 667 | N667L | 1.39 |
| 667 | N667R | 1.30 |
| 669 | P669A | 1.18 |
| 669 | P669E | 1.13 |
| 669 | P669L | 1.27 |
| 669 | P669R | 1.16 |
| 669 | P669T | 1.62 |
| 669 | P669W | 1.23 |
| 672 | V672L | 1.12 |
| 673 | Y673S | 2.06 |
| 674 | T674D | 1.14 |
| 674 | T674M | 1.36 |
| 674 | T674Q | 1.17 |
| 675 | D675A | 1.21 |
| 675 | D675V | 1.17 |
| 675 | D675Y | 1.17 |
| 676 | A676L | 1.14 |
| 676 | A676P | 1.46 |
| 677 | A677T | 1.19 |
| 679 | V679Q | 1.25 |
| 679 | V679S | 1.32 |
| 680 | K680I | 1.42 |
| 680 | K680N | 1.23 |
| 682 | Y682V | 1.20 |
| 683 | F683W | 1.18 |
| 684 | T684R | 1.17 |
| 686 | K686E | 1.11 |
| 687 | G687P | 1.49 |
| 688 | S688K | 1.36 |
| 688 | S688L | 2.15 |
| 689 | T689G | 1.26 |
| 689 | T689W | 1.39 |
| 690 | E690D | 1.35 |
| 691 | K691E | 1.10 |
| 691 | K691H | 1.16 |
| 691 | K691P | 1.26 |
| 691 | K691R | 1.26 |
| 692 | R692G | 1.35 |
| 692 | R692H | 1.48 |
| 692 | R692I | 1.22 |
| 692 | R692L | 1.16 |
| 692 | R692P | 1.12 |
| 692 | R692S | 1.24 |
| 692 | R692T | 1.26 |
| 692 | R692V | 1.12 |
| 692 | R692W | 1.15 |
| 693 | L693M | 1.11 |
| 693 | L693P | 1.16 |
| 694 | I694L | 1.61 |
| 694 | I694W | 1.58 |
| 695 | G695C | 1.84 |
| 695 | G695K | 1.15 |
| 695 | G695L | 2.14 |
| 695 | G695R | 1.21 |
| 695 | G695W | 1.42 |
| 696 | E696R | 1.26 |
| 697 | K697A | 1.22 |
| 697 | K697G | 1.30 |
| 697 | K697R | 1.54 |
| 697 | K697V | 1.12 |
| 697 | K697W | 1.13 |
| 698 | S698D | 1.15 |
| 698 | S698E | 1.73 |
| 698 | S698M | 1.38 |
| 698 | S698Q | 1.10 |
| 698 | S698R | 1.14 |
| 700 | T700A | 1.19 |
| 700 | T700D | 1.17 |
| 700 | T700Y | 1.13 |
| 701 | K701A | 2.49 |
| 701 | K701D | 1.27 |
| 701 | K701G | 1.68 |
| 701 | K701H | 1.10 |
| 701 | K701L | 1.23 |
| 701 | K701M | 1.90 |
| 701 | K701P | 1.20 |
| 701 | K701S | 1.31 |
| 703 | T703K | 1.16 |
| 705 | A705D | 1.11 |
| 705 | A705K | 1.18 |
| 705 | A705P | 1.14 |
| 705 | A705R | 1.27 |
| 705 | A705W | 1.47 |
| 706 | A706C | 1.23 |
| 706 | A706T | 1.21 |
| 706 | A706W | 1.13 |
| 708 | Y708F | 1.35 |
| 708 | Y708L | 1.11 |
| 710 | Y710E | 1.12 |
| 710 | Y710T | 1.97 |
| 710 | Y710V | 1.21 |
| 711 | Q711A | 1.26 |
| 711 | Q711D | 1.17 |
| 711 | Q711T | 1.11 |
| 712 | V712M | 1.41 |
| 712 | V712P | 1.26 |
| 712 | V712Q | 1.30 |
| 713 | Y713E | 1.14 |
| 713 | Y713G | 1.29 |
| 714 | E714H | 1.20 |
| 714 | E714I | 1.14 |
| 714 | E714K | 1.93 |
| 714 | E714N | 1.18 |
| 715 | G715K | 1.27 |
| 716 | A716M | 1.12 |
| 716 | A716P | 1.15 |
| 716 | A716R | 1.47 |
| 716 | A716T | 1.22 |
| 717 | D717G | 1.59 |
| 717 | D717L | 1.16 |
| 717 | D717S | 1.45 |
| 718 | K718A | 1.56 |
| 718 | K718Q | 1.25 |
| 718 | K718T | 1.39 |
| 718 | K718W | 1.19 |
| 718 | K718Y | 1.24 |
| 719 | D719E | 1.33 |
| 719 | D719S | 1.12 |
| 719 | D719T | 1.52 |
| 719 | D719V | 1.57 |
| 720 | S720A | 1.24 |
| 720 | S720M | 1.18 |
| 720 | S720R | 1.17 |
| 720 | S720Y | 1.13 |
| 721 | T721D | 1.16 |
| 721 | T721H | 1.35 |
| 721 | T721K | 1.13 |
| 721 | T721L | 1.33 |
| 721 | T721N | 1.45 |
| 721 | T721P | 1.13 |
| 721 | T721Q | 1.15 |
| 721 | T721S | 2.10 |
| 721 | T721V | 1.38 |
| 721 | T721W | 1.47 |
| 721 | T721Y | 1.18 |
| 722 | A722Q | 1.16 |
| 722 | A722T | 1.21 |
| 722 | A722V | 1.29 |
| 723 | H723G | 1.42 |
| 723 | H723P | 1.28 |
| 724 | K724A | 1.16 |
| 724 | K724G | 1.17 |
| 725 | N725A | 1.10 |
| 725 | N725I | 1.19 |
| 726 | M726D | 1.40 |
| 726 | M726Q | 1.11 |
| 727 | Y727L | 1.39 |
| 728 | L728C | 1.18 |
| 728 | L728K | 1.16 |
| 728 | L728S | 1.50 |
| 728 | L728W | 1.19 |
| 729 | T729A | 1.19 |
| 729 | T729E | 1.11 |
| 729 | T729M | 1.28 |
| 731 | N731E | 1.30 |
| 731 | N731Q | 1.22 |
| 732 | V732R | 1.40 |
| 733 | P733W | 1.25 |
| 734 | W734G | 1.43 |
| 734 | W734R | 1.10 |
| 735 | A735Q | 1.43 |
| 735 | A735R | 1.21 |
| 735 | A735S | 1.54 |
| 735 | A735T | 1.11 |
| 737 | G737F | 1.25 |
| 737 | G737K | 1.18 |
| 737 | G737N | 1.15 |
| 737 | G737Y | 1.14 |
| 738 | T738L | 1.16 |
| 738 | T738S | 1.34 |
| 739 | I739E | 1.15 |
| 739 | I739K | 1.50 |
| 740 | S740D | 1.47 |
| 740 | S740F | 1.20 |
| 741 | A741I | 1.51 |
| 741 | A741P | 1.21 |
| 741 | A741S | 1.30 |
| 741 | A741V | 1.16 |
| 742 | E742V | 1.14 |
| 743 | A743E | 1.15 |
| 743 | A743I | 1.16 |
| 744 | Y744L | 1.15 |
| 744 | Y744R | 1.32 |
| 745 | D745C | 1.29 |
| 745 | D745N | 1.34 |
| 745 | D745R | 1.10 |
| 746 | E746C | 1.11 |
| 746 | E746K | 1.16 |
| 746 | E746T | 1.12 |
| 747 | N747E | 1.17 |
| 747 | N747F | 1.25 |
| 747 | N747P | 1.12 |
| 749 | R749M | 1.11 |
| 750 | L750M | 1.14 |
| 750 | L750P | 1.15 |
| 750 | L750Q | 1.14 |
| 750 | L750S | 2.42 |
| 751 | I751C | 1.25 |
| 751 | I751H | 1.13 |
| 752 | P752C | 1.68 |
| 752 | P752Y | 1.51 |
| 754 | G754H | 1.26 |
| 754 | G754I | 1.15 |
| 754 | G754R | 1.20 |
| 755 | S755T | 1.15 |
| 756 | T756N | 1.88 |
| 756 | T756Q | 1.14 |
| 757 | E757A | 1.99 |
| 758 | G758V | 1.14 |
| 759 | N759S | 1.28 |
| 759 | N759V | 1.24 |
| 760 | A760G | 1.19 |
| 761 | S761A | 1.17 |
| 762 | V762W | 1.13 |
| 765 | T765P | 1.13 |
| 766 | G766M | 1.21 |
| 769 | A769F | 1.84 |
| 769 | A769G | 1.27 |
| 769 | A769I | 1.62 |
| 769 | A769N | 1.86 |
| 769 | A769V | 2.29 |
| 769 | A769Y | 1.46 |
| 770 | K770H | 1.90 |
| 771 | L771A | 1.31 |
| 772 | K772A | 1.55 |
| 772 | K772C | 1.40 |
| 772 | K772P | 2.23 |
| 772 | K772W | 1.23 |
| 773 | A773C | 1.42 |
| 773 | A773M | 1.36 |
| 773 | A773R | 1.34 |
| 774 | D774A | 1.43 |
| 775 | A775V | 1.15 |
| 776 | D776R | 1.15 |
| 776 | D776V | 1.32 |
| 777 | R777H | 1.62 |
| 777 | R777S | 1.41 |
| 777 | R777T | 1.15 |
| 778 | K778F | 1.30 |
| 778 | K778G | 1.22 |
| 778 | K778N | 1.34 |
| 778 | K778R | 1.18 |
| 779 | T779I | 1.14 |
| 781 | T781A | 1.81 |
| 781 | T781E | 1.10 |
| 781 | T781F | 1.36 |
| 781 | T781G | 1.65 |
| 781 | T781P | 1.60 |
| 781 | T781Y | 1.59 |
| 782 | A782C | 1.17 |
| 782 | A782N | 1.19 |
| 782 | A782Q | 1.47 |
| 782 | A782Y | 1.28 |
| 783 | D783A | 1.32 |
| 785 | K785C | 1.30 |
| 785 | K785I | 1.15 |
| 785 | K785S | 1.68 |
| 785 | K785V | 1.14 |
| 785 | K785W | 2.09 |
| 785 | K785Y | 1.31 |
| 786 | D786V | 1.58 |
| 787 | L787D | 1.12 |
| 787 | L787K | 1.13 |
| 789 | Y789V | 1.41 |
| 790 | I790R | 1.34 |
| 790 | I790V | 1.15 |
| 791 | E791D | 1.11 |
| 791 | E791F | 1.11 |
| 791 | E791S | 1.26 |
| 791 | E791V | 1.11 |
| 791 | E791W | 1.17 |
| 792 | V792S | 1.21 |
| 792 | V792Y | 1.20 |
| 793 | D793C | 1.12 |
| 793 | D793Y | 1.10 |
| 794 | V794D | 1.11 |
| 796 | D796S | 1.12 |
| 796 | D796T | 1.13 |
| 797 | A797H | 1.10 |
| 798 | N798I | 1.44 |
| 798 | N798Q | 1.10 |
| 799 | G799D | 1.10 |
| 799 | G799K | 1.51 |
| 799 | G799L | 1.58 |
| 799 | G799Y | 1.19 |
| 800 | H800F | 1.14 |
| 800 | H800G | 1.16 |
| 800 | H800V | 1.24 |
| 801 | I801W | 1.11 |
| 802 | V802E | 1.20 |
| 802 | V802I | 1.27 |
| 802 | V802S | 1.22 |
| 802 | V802Y | 1.31 |
| 803 | P803G | 1.19 |
| 803 | P803S | 1.69 |
| 803 | P803Y | 1.22 |
| 804 | D804E | 1.10 |
| 805 | A805C | 1.13 |
| 805 | A805F | 1.25 |
| 805 | A805I | 1.11 |
| 805 | A805N | 1.11 |
| 805 | A805P | 1.19 |
| 806 | A806F | 1.20 |
| 806 | A806I | 1.32 |
| 806 | A806Q | 1.43 |
| 807 | N807F | 1.10 |
| 808 | R808F | 1.39 |
| 808 | R808G | 1.26 |
| 808 | R808I | 1.20 |
| 808 | R808P | 1.11 |
| 809 | V809L | 1.20 |
| 809 | V809M | 1.28 |
| 809 | V809P | 1.18 |
| 810 | T810L | 1.18 |
| 810 | T810P | 1.98 |
| 810 | T810Q | 1.35 |
| 810 | T810R | 1.10 |
| 810 | T810Y | 1.24 |
| 811 | F811L | 1.21 |
| 813 | V813H | 1.11 |
| 813 | V813W | 1.39 |
| 813 | V813Y | 1.11 |
| 814 | K814G | 1.22 |
| 814 | K814P | 1.27 |
| 815 | G815F | 1.11 |
| 815 | G815M | 1.29 |
| 815 | G815P | 1.20 |
| 816 | A816D | 1.22 |
| 816 | A816N | 1.13 |
| 816 | A816W | 1.21 |
| 817 | G817H | 1.18 |
| 818 | K818D | 1.21 |
| 818 | K818F | 1.53 |
| 818 | K818L | 1.46 |
| 818 | K818Q | 1.22 |
| 818 | K818R | 1.18 |
| 818 | K818S | 1.13 |
| 818 | K818W | 1.53 |
| 818 | K818Y | 1.35 |
| 819 | L819F | 1.40 |
| 820 | V820C | 1.24 |
| 820 | V820R | 1.22 |
| 820 | V820W | 1.15 |
| 821 | G821I | 1.52 |
| 822 | V822A | 1.25 |
| 822 | V822D | 1.78 |
| 822 | V822E | 1.42 |
| 822 | V822T | 1.13 |
| 823 | D823E | 1.12 |
| 824 | N824A | 1.14 |
| 825 | G825A | 1.21 |
| 826 | S826A | 1.19 |
| 826 | S826G | 1.30 |
| 826 | S826I | 1.12 |
| 826 | S826W | 1.17 |
| 828 | P828G | 1.15 |
| 829 | D829C | 1.12 |
| 829 | D829I | 1.15 |
| 829 | D829S | 1.32 |
| 830 | H830Q | 1.14 |
| 831 | D831A | 1.14 |
| 831 | D831M | 1.12 |
| 831 | D831P | 1.44 |
| 832 | S832P | 1.19 |
| 832 | S832R | 1.47 |
| 832 | S832W | 1.15 |
| 833 | Y833C | 1.14 |
| 833 | Y833D | 1.17 |
| 833 | Y833E | 1.27 |
| 833 | Y833N | 1.16 |
| 834 | Q834M | 1.21 |
| 835 | A835D | 1.90 |
| 835 | A835H | 1.80 |
| 835 | A835K | 1.33 |
| 836 | D836C | 1.37 |
| 836 | D836E | 1.52 |
| 836 | D836Q | 1.21 |
| 836 | D836R | 1.13 |
| 836 | D836S | 1.74 |
| 836 | D836T | 1.26 |
| 836 | D836V | 1.34 |
| 836 | D836Y | 1.79 |
| 838 | R838N | 1.33 |
| 838 | R838S | 1.17 |
| 839 | K839A | 1.32 |
| 839 | K839D | 1.63 |
| 839 | K839L | 1.14 |
| 839 | K839N | 1.18 |
| 840 | A840G | 1.43 |
| 840 | A840I | 1.26 |
| 840 | A840P | 1.37 |
| 840 | A840V | 1.32 |
| 841 | F841I | 1.19 |
| 841 | F841K | 1.17 |
| 842 | S842A | 1.17 |
| 844 | K844A | 1.27 |
| 844 | K844Y | 1.14 |
| 847 | A847T | 1.15 |
| 848 | I848M | 1.16 |
| 850 | Q850L | 1.17 |
| 851 | S851C | 1.18 |
| 851 | S851D | 1.17 |
| 851 | S851E | 1.13 |
| 851 | S851T | 1.12 |
| 852 | T852L | 1.14 |
| 853 | K853P | 1.26 |
| 854 | E854C | 1.58 |
| 854 | E854I | 1.26 |
| 855 | A855K | 1.10 |
| 855 | A855V | 1.15 |
| 855 | A855Y | 1.12 |
| 858 | I858D | 1.46 |
| 858 | I858G | 1.14 |
| 858 | I858K | 1.28 |
| 858 | I858P | 1.21 |
| 860 | V860Y | 1.11 |
| 861 | T861F | 1.23 |
| 861 | T861I | 1.29 |
| 861 | T861Q | 1.14 |
| 861 | T861V | 1.14 |
| 861 | T861W | 1.35 |
| 862 | A862C | 1.11 |
| 864 | A864K | 1.16 |
| 865 | D865A | 1.49 |
| 865 | D865G | 1.71 |
| 866 | G866H | 1.30 |
| 866 | G866R | 1.25 |
| 867 | L867W | 1.73 |
| 870 | S870E | 1.30 |
| 870 | S870M | 1.27 |
| 870 | S870T | 1.23 |
| 872 | V872C | 1.72 |
| 872 | V872G | 1.25 |
| 873 | K873G | 1.39 |
| 873 | K873Y | 1.38 |
| 874 | I874G | 1.43 |
| 875 | A875R | 1.13 |
| 879 | V879S | 1.20 |
| 882 | T882A | 1.26 |
| 882 | T882M | 1.15 |
| 882 | T882R | 1.23 |
| 883 | S883L | 1.50 |
| 885 | E885V | 1.27 |
| 886 | K886L | 1.30 |
| 886 | K886V | 1.34 |
| 886 | K886W | 1.33 |
| 887 | T887A | 1.27 |
| 887 | T887D | 1.28 |
| 887 | T887G | 1.40 |
| 887 | T887N | 1.25 |
| 888 | V888A | 1.42 |
| 889 | R889G | 1.23 |
| 892 | Y892P | 1.43 |
| 893 | Y893E | 1.55 |
| 893 | Y893G | 1.18 |
| 894 | S894D | 1.30 |
| 894 | S894G | 1.18 |
| 897 | Y897V | 1.32 |
| 898 | Y898T | 1.11 |
| 900 | K900E | 1.36 |
| 900 | K900G | 1.12 |
| 901 | T901Q | 1.32 |
| 901 | T901R | 1.35 |
| 901 | T901Y | 1.56 |
| 902 | G902A | 1.22 |
| 902 | G902D | 1.10 |
| 902 | G902F | 1.38 |
| 902 | G902P | 1.22 |
| 902 | G902Q | 1.21 |
| 902 | G902R | 1.47 |
| 902 | G902S | 1.77 |
| 902 | G902W | 1.41 |
| 903 | N903D | 1.42 |
| 903 | N903I | 1.25 |
| 904 | K904D | 1.24 |
| 904 | K904E | 1.21 |
| 904 | K904M | 1.44 |
| 904 | K904N | 1.32 |
| 904 | K904R | 1.24 |
| 904 | K904S | 1.32 |
| 904 | K904V | 1.52 |
| 904 | K904W | 1.20 |
| 905 | P905A | 1.16 |
| 905 | P905C | 1.37 |
| 905 | P905R | 1.48 |
| 905 | P905V | 1.10 |
| 905 | P905W | 1.29 |
| 905 | P905Y | 1.45 |
| 906 | I906A | 1.12 |
| 906 | I906N | 1.25 |
| 906 | I906T | 1.12 |
| 906 | I906W | 1.44 |
| 907 | L907F | 1.34 |
| 907 | L907S | 1.23 |
| 908 | P908C | 1.20 |
| 908 | P908D | 1.22 |
| 908 | P908G | 1.26 |
| 908 | P908I | 1.40 |
| 908 | P908L | 1.37 |
| 908 | P908T | 1.16 |
| 909 | S909E | 1.18 |
| 909 | S909F | 1.14 |
| 909 | S909G | 1.11 |
| 909 | S909W | 1.13 |
| 909 | S909Y | 1.19 |
| 910 | D910I | 1.11 |
| 910 | D910W | 1.47 |
| 911 | V911A | 1.19 |
| 912 | E912A | 1.15 |
| 912 | E912K | 1.16 |
| 912 | E912L | 1.20 |
| 912 | E912T | 2.02 |
| 912 | E912V | 1.12 |
| 913 | V913G | 1.11 |
| 913 | V913R | 1.11 |
| 914 | R914A | 1.81 |
| 914 | R914E | 1.11 |
| 914 | R914F | 1.84 |
| 914 | R914I | 1.34 |
| 914 | R914K | 1.76 |
| 914 | R914V | 1.36 |
| 914 | R914W | 1.21 |
| 914 | R914Y | 1.53 |
| 915 | Y915A | 1.46 |
| 915 | Y915C | 1.52 |
| 915 | Y915G | 1.33 |
| 915 | Y915I | 1.21 |
| 915 | Y915M | 1.70 |
| 915 | Y915Q | 1.36 |
| 915 | Y915V | 1.58 |
| 916 | S916G | 1.73 |
| 917 | D917C | 1.47 |
| 917 | D917E | 1.25 |
| 917 | D917F | 1.72 |
| 917 | D917L | 1.29 |
| 917 | D917M | 1.21 |
| 917 | D917Q | 1.26 |
| 917 | D917S | 1.80 |
| 918 | G918E | 1.40 |
| 918 | G918H | 1.53 |
| 918 | G918T | 1.33 |
| 918 | G918V | 1.11 |
| 919 | T919D | 1.31 |
| 919 | T919K | 1.16 |
| 919 | T919Q | 1.93 |
| 919 | T919W | 1.23 |
| 920 | S920P | 1.17 |
| 920 | S920V | 1.16 |
| 921 | D921C | 1.16 |
| 921 | D921V | 1.54 |
| 922 | R922A | 1.95 |
| 922 | R922M | 1.30 |
| 922 | R922V | 1.19 |
| 922 | R922W | 1.26 |
| 923 | Q923A | 1.21 |
| 923 | Q923E | 1.10 |
| 923 | Q923L | 1.15 |
| 923 | Q923M | 1.10 |
| 923 | Q923V | 2.01 |
| 924 | N924Q | 1.11 |
| 924 | N924S | 1.11 |
| 925 | V925C | 1.29 |
| 925 | V925E | 1.15 |
| 925 | V925G | 1.17 |
| 925 | V925K | 1.28 |
| 925 | V925N | 1.11 |
| 925 | V925W | 1.10 |
| 926 | T926P | 1.15 |
| 926 | T926R | 1.32 |
| 927 | W927P | 1.40 |
| 928 | D928A | 1.27 |
| 928 | D928H | 1.13 |
| 928 | D928Q | 1.21 |
| 929 | A929P | 1.31 |
| 930 | V930E | 1.15 |
| 930 | V930I | 1.22 |
| 930 | V930T | 1.11 |
| 931 | S931P | 1.11 |
| 931 | S931R | 1.30 |
| 933 | D933R | 1.33 |
| 934 | Q934S | 1.56 |
| 934 | Q934V | 1.30 |
| 935 | I935A | 1.30 |
| 935 | I935C | 1.32 |
| 935 | I935D | 1.15 |
| 935 | I935E | 1.40 |
| 935 | I935L | 1.19 |
| 935 | I935P | 1.54 |
| 935 | I935V | 1.20 |
| 935 | I935W | 1.32 |
| 936 | A936I | 1.39 |
| 936 | A936L | 1.11 |
| 936 | A936Q | 1.54 |
| 936 | A936R | 1.13 |
| 936 | A936Y | 1.26 |
| 937 | K937G | 1.34 |
| 937 | K937I | 1.64 |
| 937 | K937M | 1.28 |
| 937 | K937P | 1.55 |
| 937 | K937Q | 1.32 |
| 938 | A938C | 1.46 |
| 938 | A938H | 1.18 |
| 938 | A938N | 1.15 |
| 938 | A938T | 1.21 |
| 938 | A938V | 1.14 |
| 938 | A938W | 1.15 |
| 939 | G939D | 1.30 |
| 939 | G939K | 1.17 |
| 940 | S940C | 1.34 |
| 940 | S940E | 1.26 |
| 940 | S940R | 1.21 |
| 940 | S940V | 1.25 |
| 941 | F941C | 1.32 |
| 941 | F941M | 1.17 |
| 942 | S942A | 1.19 |
| 942 | S942E | 1.20 |
| 942 | S942K | 1.46 |
| 942 | S942P | 1.17 |
| 942 | S942V | 1.41 |
| 943 | V943A | 1.60 |
| 943 | V943G | 1.20 |
| 943 | V943Q | 1.19 |
| 946 | T946A | 1.23 |
| 946 | T946L | 1.13 |
| 946 | T946P | 1.44 |
| 946 | T946W | 1.25 |
| 947 | V947G | 1.46 |
| 947 | V947L | 1.26 |
| 947 | V947P | 1.28 |
| 947 | V947R | 1.37 |
| 947 | V947T | 1.36 |
| 948 | A948I | 1.21 |
| 948 | A948W | 1.36 |
| 949 | G949A | 1.24 |
| 949 | G949V | 1.11 |
| 950 | Q950K | 1.15 |
| 950 | Q950W | 1.16 |
| 951 | K951D | 1.30 |
| 951 | K951G | 1.19 |
| 951 | K951P | 1.18 |
| 951 | K951S | 1.36 |
| 951 | K951W | 1.23 |
| 951 | K951Y | 1.21 |
| 952 | I952Q | 1.15 |
| 953 | S953F | 1.22 |
| 953 | S953M | 1.37 |
| 953 | S953N | 1.15 |
| 953 | S953R | 1.29 |
| 954 | V954D | 1.23 |
| 954 | V954Q | 1.29 |
| 955 | R955A | 1.12 |
| 955 | R955C | 1.36 |
| 955 | R955K | 1.18 |
| 955 | R955Q | 1.19 |
| 955 | R955W | 1.35 |
| 956 | V956A | 1.13 |
| 956 | V956D | 1.31 |
| 956 | V956G | 1.12 |
| 956 | V956H | 1.13 |
| 956 | V956I | 1.17 |
| 956 | V956M | 1.18 |
| 956 | V956Q | 1.57 |
| 956 | V956W | 1.19 |
| 957 | T957D | 1.12 |
| 957 | T957S | 1.21 |
| 957 | T957W | 1.17 |
| 959 | I959A | 1.20 |
| 959 | I959L | 1.14 |
| 959 | I959S | 1.20 |
| 959 | I959V | 1.20 |
| 959 | I959Y | 1.30 |
| 960 | D960P | 1.13 |
| 960 | D960S | 1.25 |
| 961 | E961D | 1.52 |
| 961 | E961F | 1.91 |
| 961 | E961K | 1.18 |
| 961 | E961S | 1.53 |
| 961 | E961T | 1.21 |
| 962 | I962A | 1.25 |
| 962 | I962C | 1.44 |
| 962 | I962D | 1.11 |
| 962 | I962G | 1.13 |
| 962 | I962K | 1.32 |
| 962 | I962N | 1.43 |
| 963 | G963C | 1.18 |
| 963 | G963E | 1.12 |
| 963 | G963P | 1.12 |
| 965 | L965C | 1.55 |
| 965 | L965E | 1.16 |
| 965 | L965G | 1.50 |
| 965 | L965K | 1.32 |
| 965 | L965M | 1.56 |
| 965 | L965P | 1.50 |
| 965 | L965Q | 1.19 |
| 965 | L965S | 1.13 |
| 965 | L965V | 1.27 |
| 965 | L965Y | 1.53 |
| 966 | L966A | 1.46 |
| 966 | L966K | 1.13 |
| 966 | L966N | 1.28 |
| 966 | L966Q | 1.16 |
| 966 | L966S | 1.13 |
| 966 | L966V | 1.10 |
| 967 | N967D | 1.28 |
| 967 | N967I | 1.17 |
| 967 | N967L | 1.13 |
| 967 | N967M | 1.33 |
| 967 | N967P | 1.27 |
| 967 | N967T | 1.23 |
| 967 | N967V | 1.15 |
| 968 | Y968G | 1.52 |
| 968 | Y968L | 1.30 |
| 968 | Y968Q | 1.33 |
| 968 | Y968V | 1.48 |
| 969 | S969C | 1.42 |
| 969 | S969D | 1.23 |
| 969 | S969G | 1.19 |
| 969 | S969H | 1.30 |
| 969 | S969I | 1.67 |
| 969 | S969L | 1.39 |
| 969 | S969M | 1.72 |
| 969 | S969P | 1.18 |
| 969 | S969Q | 1.36 |
| 969 | S969Y | 1.27 |
| 970 | A970I | 1.32 |
| 970 | A970L | 1.17 |
| 971 | S971G | 1.15 |
| 971 | S971V | 1.30 |
| 971 | S971W | 1.30 |
| 973 | P973C | 1.74 |
| 973 | P973D | 1.22 |
| 973 | P973K | 1.19 |
| 973 | P973N | 1.23 |
| 973 | P973Q | 1.13 |
| 973 | P973R | 1.26 |
| 973 | P973V | 1.10 |
| 973 | P973W | 1.38 |
| 973 | P973Y | 1.10 |
| 974 | V974C | 1.14 |
| 974 | V974E | 1.23 |
| 974 | V974G | 1.16 |
| 974 | V974N | 1.11 |
| 974 | V974T | 1.35 |
| 974 | V974Y | 1.23 |
| 975 | G975F | 1.11 |
| 975 | G975K | 1.19 |
| 976 | T976D | 1.11 |
| 976 | T976F | 1.21 |
| 976 | T976G | 1.25 |
| 976 | T976K | 1.45 |
| 976 | T976L | 1.11 |
| 976 | T976P | 1.41 |
| 976 | T976S | 1.11 |
| 977 | P977C | 1.35 |
| 977 | P977K | 1.16 |
| 977 | P977R | 1.16 |
| 977 | P977T | 1.49 |
| 977 | P977Y | 1.23 |
| 978 | A978F | 1.31 |
| 978 | A978G | 1.11 |
| 978 | A978M | 1.42 |
| 978 | A978N | 1.27 |
| 978 | A978R | 1.27 |
| 978 | A978S | 1.16 |
| 978 | A978Y | 1.17 |
| 979 | V979G | 1.11 |
| 979 | V979N | 1.14 |
| 979 | V979Y | 1.16 |
| 980 | L980A | 1.28 |
| 980 | L980F | 1.15 |
| 980 | L980H | 1.44 |
| 980 | L980I | 1.29 |
| 980 | L980K | 1.13 |
| 980 | L980N | 1.46 |
| 980 | L980Q | 1.30 |
| 980 | L980T | 1.28 |
| 980 | L980Y | 1.15 |
| 981 | P981L | 1.13 |
| 981 | P981M | 1.55 |
| 984 | R984P | 1.13 |
| 985 | P985F | 1.12 |
| 985 | P985H | 1.13 |
| 985 | P985K | 1.14 |
| 985 | P985L | 2.25 |
| 985 | P985W | 1.49 |
| 986 | A986C | 1.15 |
| 986 | A986I | 1.88 |
| 986 | A986L | 1.38 |
| 986 | A986M | 1.17 |
| 986 | A986W | 1.30 |
| 987 | V987A | 1.31 |
| 987 | V987C | 1.18 |
| 987 | V987F | 1.31 |
| 987 | V987K | 1.43 |
| 988 | L988V | 1.13 |
| 989 | P989M | 1.23 |
| 989 | P989W | 1.12 |
| 990 | D990F | 1.11 |
| 990 | D990P | 1.18 |
| 990 | D990W | 1.50 |
| 991 | G991C | 1.18 |
| 991 | G991F | 1.23 |
| 991 | G991H | 1.25 |
| 991 | G991K | 1.15 |
| 991 | G991P | 1.17 |
| 991 | G991Y | 1.38 |
| 992 | T992M | 1.29 |
| 992 | T992N | 1.11 |
| 993 | V993G | 1.14 |
| 995 | S995E | 1.15 |
| 995 | S995L | 1.41 |
| 995 | S995R | 1.18 |
| 996 | A996R | 1.16 |
| 997 | N997K | 1.44 |
| 997 | N997V | 1.15 |
| 998 | F998M | 1.25 |
| 998 | F998W | 1.21 |
| 1000 | V1000W | 1.20 |
| 1001 | D1001L | 1.45 |
| 1001 | D1001S | 1.20 |
| 1002 | W1002A | 1.61 |
| 1002 | W1002D | 1.72 |
| 1002 | W1002E | 1.30 |
| 1002 | W1002H | 1.42 |
| 1002 | W1002N | 1.77 |
| 1002 | W1002P | 1.66 |
| 1002 | W1002Q | 1.32 |
| 1002 | W1002S | 1.20 |
| 1003 | T1003F | 1.17 |
| 1003 | T1003G | 1.20 |
| 1003 | T1003N | 1.15 |
| 1003 | T1003R | 1.19 |
| 1003 | T1003S | 1.17 |
| 1003 | T1003W | 1.13 |
| 1003 | T1003Y | 1.40 |
| 1004 | K1004D | 1.15 |
| 1004 | K1004E | 1.13 |
| 1004 | K1004S | 1.16 |
| 1005 | P1005I | 1.20 |
| 1005 | P1005N | 1.11 |
| 1005 | P1005V | 1.19 |
| 1005 | P1005Y | 1.31 |
| 1006 | A1006C | 1.31 |
| 1006 | A1006N | 1.11 |
| 1006 | A1006P | 1.13 |
| 1006 | A1006S | 2.01 |
| 1006 | A1006V | 1.10 |
| 1006 | A1006W | 1.17 |
| 1006 | A1006Y | 1.16 |
| 1007 | D1007C | 1.16 |
| 1007 | D1007L | 1.24 |
| 1007 | D1007P | 1.61 |
| 1007 | D1007V | 1.21 |
| 1008 | T1008G | 1.19 |
| 1010 | Y1010A | 1.15 |
| 1010 | Y1010P | 1.10 |
| 1010 | Y1010R | 1.10 |
| 1010 | Y1010T | 1.21 |
| 1011 | N1011A | 1.20 |
| 1011 | N1011S | 1.10 |
| 1011 | N1011T | 1.35 |
| 1011 | N1011W | 1.11 |
| 1012 | T1012H | 1.11 |
| 1012 | T1012I | 1.17 |
| 1012 | T1012Q | 1.31 |
| 1013 | A1013D | 1.18 |
| 1013 | A1013Q | 1.28 |
| 1013 | A1013V | 1.24 |
| 1014 | G1014I | 1.35 |
| 1014 | G1014M | 1.13 |
| 1014 | G1014V | 1.18 |
| 1014 | G1014W | 1.17 |
| 1015 | T1015A | 1.11 |
| 1016 | V1016D | 1.21 |
| 1016 | V1016P | 1.10 |
| 1017 | K1017E | 1.11 |
| 1017 | K1017G | 1.12 |
| 1018 | V1018K | 1.48 |
| 1018 | V1018L | 1.18 |
| 1018 | V1018R | 1.11 |
| 1018 | V1018S | 1.12 |
| 1021 | T1021C | 1.10 |
| 1021 | T1021E | 1.11 |
| 1021 | T1021G | 1.24 |
| 1022 | A1022H | 1.41 |
| 1024 | V1024G | 1.40 |
| 1024 | V1024H | 1.35 |
| 1024 | V1024K | 1.27 |
| 1024 | V1024R | 1.24 |
| 1024 | V1024S | 1.20 |
| 1024 | V1024W | 1.25 |
| 1026 | G1026E | 1.14 |
| 1026 | G1026S | 1.30 |
| 1026 | G1026V | 1.32 |
| 1026 | G1026Y | 1.18 |
| 1027 | K1027Q | 1.13 |
| 1027 | K1027R | 1.13 |
| 1028 | E1028G | 1.10 |
| 1028 | E1028T | 1.11 |
| 1029 | F1029I | 1.10 |
| 1029 | F1029K | 1.20 |
| 1029 | F1029P | 1.21 |
| 1029 | F1029V | 1.22 |
| 1030 | K1030D | 1.36 |
| 1030 | K1030H | 1.14 |
| 1030 | K1030M | 1.20 |
| 1030 | K1030W | 1.59 |
| 1031 | V1031H | 1.29 |
| 1031 | V1031K | 1.27 |
| 1033 | A1033V | 1.15 |
| 1034 | T1034G | 1.14 |
| 1034 | T1034H | 1.23 |
| 1034 | T1034N | 1.14 |
| 1035 | I1035D | 1.51 |
| 1035 | I1035G | 1.10 |
| 1035 | I1035Q | 1.13 |
| 1036 | R1036G | 1.21 |
| 1036 | R1036L | 1.23 |
| 1036 | R1036T | 1.29 |
| 1036 | R1036Y | 1.11 |
| 1037 | V1037C | 1.11 |
| 1038 | Q1038D | 1.16 |
| 1039 | R1039V | 1.11 |
| 1040 | S1040M | 1.14 |
| 1041 | Q1041P | 1.33 |
| 1042 | V1042N | 1.32 |
| 1043 | T1043F | 1.22 |
| 1043 | T1043G | 1.16 |
| 1043 | T1043R | 1.17 |
| 1044 | I1044A | 1.30 |
| 1044 | I1044L | 1.18 |
| 1046 | S1046M | 1.27 |
| 1048 | V1048C | 1.31 |
| 1048 | V1048F | 1.21 |
| 1048 | V1048G | 1.38 |
| 1048 | V1048I | 1.19 |
| 1048 | V1048M | 1.46 |
| 1048 | V1048Q | 1.60 |
| 1051 | N1051A | 1.10 |
| 1051 | N1051E | 2.06 |
| 1051 | N1051K | 1.33 |
| 1052 | A1052C | 1.10 |
| 1052 | A1052K | 1.42 |
| 1052 | A1052R | 1.18 |
| 1053 | L1053A | 1.57 |
| 1053 | L1053W | 1.26 |
| 1054 | R1054C | 1.17 |
| 1054 | R1054L | 1.14 |
| 1055 | L1055R | 1.21 |
| 1055 | L1055T | 1.19 |
| 1057 | Q1057A | 1.16 |
| 1057 | Q1057E | 1.48 |
| 1057 | Q1057P | 1.14 |
| 1058 | N1058R | 1.26 |
| 1058 | N1058S | 1.24 |
| 1060 | P1060G | 1.29 |
| 1060 | P1060N | 1.23 |
| 1060 | P1060Q | 1.14 |
| 1061 | A1061G | 1.33 |
| 1061 | A1061K | 1.17 |
| 1061 | A1061W | 1.15 |
| 1062 | D1062A | 1.21 |
| 1062 | D1062G | 1.16 |
| 1062 | D1062I | 1.17 |
| 1062 | D1062M | 1.32 |
| 1063 | K1063M | 1.14 |
| 1064 | Q1064M | 1.34 |
| 1064 | Q1064R | 1.22 |
| 1064 | Q1064T | 1.10 |
| 1064 | Q1064V | 1.19 |
| 1065 | S1065A | 1.38 |
| 1065 | S1065C | 1.23 |
| 1066 | D1066A | 1.30 |
| 1066 | D1066G | 1.28 |
| 1066 | D1066V | 1.34 |
| 1067 | T1067G | 1.19 |
| 1067 | T1067M | 1.35 |
| 1068 | L1068P | 1.38 |
| 1068 | L1068Q | 1.29 |
| 1068 | L1068Y | 1.15 |
| 1069 | D1069R | 1.10 |
| 1070 | A1070T | 1.15 |
| 1071 | I1071M | 1.13 |
| 1072 | K1072P | 1.15 |
| 1072 | K1072S | 1.34 |
| 1073 | D1073F | 1.17 |
| 1073 | D1073W | 1.18 |
| 1074 | G1074L | 1.36 |
| 1074 | G1074R | 1.23 |
| 1075 | S1075G | 1.15 |
| 1075 | S1075L | 1.25 |
| 1076 | T1076E | 1.11 |
| 1076 | T1076S | 1.29 |
| 1077 | T1077R | 1.17 |
| 1078 | V1078D | 1.10 |
| 1078 | V1078W | 1.10 |
| 1079 | D1079G | 1.13 |
| 1079 | D1079L | 1.14 |
| 1081 | N1081E | 1.12 |
| 1082 | T1082E | 1.21 |
| 1082 | T1082F | 1.42 |
| 1082 | T1082G | 1.11 |
| 1082 | T1082K | 1.12 |
| 1083 | G1083E | 1.41 |
| 1083 | G1083F | 1.31 |
| 1083 | G1083L | 1.35 |
| 1084 | G1084M | 1.11 |
| 1084 | G1084V | 1.73 |
| 1084 | G1084W | 1.18 |
| 1084 | G1084Y | 1.25 |
| 1085 | G1085P | 1.13 |
| 1085 | G1085R | 1.12 |
| 1085 | G1085S | 1.77 |
| 1086 | A1086H | 1.25 |
| 1086 | A1086K | 1.17 |
| 1086 | A1086R | 1.13 |
| 1087 | N1087A | 1.12 |
| 1087 | N1087E | 1.14 |
| 1087 | N1087I | 1.17 |
| 1087 | N1087R | 1.25 |
| 1088 | P1088D | 1.18 |
| 1088 | P1088E | 1.47 |
| 1088 | P1088G | 1.12 |
| 1088 | P1088R | 1.40 |
| 1088 | P1088W | 1.10 |
| 1089 | S1089G | 1.30 |
| 1089 | S1089K | 1.23 |
| 1089 | S1089Q | 1.58 |
| 1089 | S1089V | 1.15 |
| 1090 | A1090F | 1.12 |
| 1091 | W1091Y | 1.33 |
| 1093 | N1093A | 1.35 |
| 1093 | N1093G | 1.10 |
| 1093 | N1093L | 1.17 |
| 1093 | N1093P | 2.25 |
| 1093 | N1093Q | 1.40 |
| 1094 | W1094D | 1.62 |
| 1094 | W1094E | 1.51 |
| 1094 | W1094P | 1.27 |
| 1094 | W1094R | 1.13 |
| 1094 | W1094T | 1.11 |
| 1095 | A1095P | 1.12 |
| 1095 | A1095T | 1.17 |
| 1095 | A1095W | 1.22 |
| 1096 | Y1096D | 1.26 |
| 1096 | Y1096L | 1.12 |
| 1097 | S1097E | 1.15 |
| 1097 | S1097K | 1.13 |
| 1097 | S1097T | 1.28 |
| 1098 | K1098D | 1.31 |
| 1098 | K1098F | 1.24 |
| 1098 | K1098G | 1.14 |
| 1098 | K1098Q | 1.19 |
| 1098 | K1098S | 1.11 |
| 1099 | A1099D | 1.26 |
| 1099 | A1099S | 1.21 |
| 1099 | A1099V | 1.40 |
| 1099 | A1099W | 1.12 |
| 1100 | G1100E | 1.15 |
| 1100 | G1100H | 1.30 |
| 1100 | G1100N | 1.18 |
| 1101 | H1101K | 1.11 |
| 1101 | H1101Q | 1.17 |
| 1101 | H1101R | 1.12 |
| 1101 | H1101V | 1.38 |
| 1102 | N1102H | 1.66 |
| 1102 | N1102Q | 1.15 |
| 1102 | N1102T | 1.19 |
| 1103 | T1103A | 1.15 |
| 1103 | T1103E | 1.30 |
| 1103 | T1103S | 1.15 |
| 1103 | T1103W | 1.11 |
| 1105 | E1105L | 1.27 |
| 1105 | E1105S | 1.15 |
| 1109 | E1109A | 1.32 |
| 1109 | E1109D | 1.74 |
| 1109 | E1109L | 1.71 |
| 1109 | E1109W | 1.47 |
| 1111 | A1111G | 1.42 |
| 1111 | A1111S | 1.16 |
| 1113 | E1113G | 1.14 |
| 1113 | E1113P | 1.61 |
| 1113 | E1113V | 1.34 |
| 1114 | Q1114E | 1.34 |
| 1114 | Q1114L | 1.16 |
| 1114 | Q11MR | 1.38 |
| 1114 | Q1114S | 1.69 |
| 1114 | Q1114T | 1.30 |
| 1114 | Q1114V | 1.79 |
| 1115 | Q1115A | 1.56 |
| 1115 | Q1115K | 1.48 |
| 1115 | Q1115L | 1.27 |
| 1115 | Q1115P | 1.22 |
| 1116 | L1116D | 1.14 |
| 1116 | L1116G | 1.26 |
| 1116 | L1116H | 1.16 |
| 1116 | L1116K | 2.13 |
| 1116 | L1116W | 1.55 |
| 1117 | G1117E | 1.29 |
| 1117 | G1117I | 1.47 |
| 1117 | G1117M | 1.27 |
| 1117 | G1117R | 1.37 |
| 1117 | G1117S | 1.58 |
| 1117 | G1117T | 1.51 |
| 1117 | G1117V | 1.83 |
| 1117 | G1117W | 2.52 |
| 1118 | Q1118M | 1.17 |
| 1118 | Q1118W | 1.20 |
| 1119 | I1119D | 1.10 |
| 1119 | I1119E | 1.22 |
| 1119 | I1119N | 1.50 |
| 1119 | I1119S | 1.21 |
| 1120 | V1120N | 1.14 |
| 1120 | V1120S | 1.12 |
| 1121 | M1121G | 1.37 |
| 1121 | M1121K | 1.24 |
| 1121 | M1121N | 1.19 |
| 1121 | M1121P | 1.19 |
| 1121 | M1121S | 1.17 |
| 1123 | F1123E | 1.14 |
| 1123 | F1123R | 1.28 |
| 1124 | F1124V | 1.21 |
| 1124 | F1124W | 1.23 |
| 1125 | R1125D | 1.25 |
| 1125 | R1125F | 1.12 |
| 1125 | R1125K | 1.21 |
| 1125 | R1125V | 1.87 |
| 1125 | R1125W | 1.68 |
| 1127 | S1127I | 1.30 |
| 1127 | S1127Q | 1.63 |
| 1128 | N1128A | 1.51 |
| 1129 | A1129E | 1.61 |
| 1129 | A1129R | 1.11 |
| 1130 | V1130A | 1.42 |
| 1130 | V1130R | 1.60 |
| 1131 | R1131W | 1.18 |
| 1132 | F1132M | 1.18 |
| 1132 | F1132P | 1.13 |
| 1132 | F1132Q | 1.18 |
| 1133 | P1133D | 1.45 |
| 1133 | P1133G | 1.21 |
| 1133 | P1133L | 1.14 |
| 1133 | P1133R | 1.72 |
| 1135 | A1135E | 1.15 |
| 1135 | A1135K | 1.14 |
| 1135 | A1135L | 1.35 |
| 1135 | A1135W | 1.35 |
| 1136 | G1136P | 1.31 |
| 1137 | K1137G | 1.12 |
| 1137 | K1137P | 1.21 |
| 1137 | K1137R | 1.45 |
| 1137 | K1137S | 1.70 |
| 1137 | K1137T | 1.14 |
| 1137 | K1137V | 1.16 |
| 1138 | T1138R | 1.25 |
| 1138 | T1138Y | 1.18 |
| 1140 | I1140C | 1.23 |
| 1140 | I1140P | 1.33 |
| 1140 | I1140R | 1.15 |
| 1141 | Q1141A | 1.15 |
| 1141 | Q1141G | 1.11 |
| 1141 | Q1141K | 1.33 |
| 1141 | Q1141N | 1.14 |
| 1141 | Q1141T | 1.10 |
| 1142 | I1142E | 1.19 |
| 1142 | I1142R | 1.19 |
| 1142 | I1142Y | 1.39 |
| 1143 | S1143G | 1.23 |
| 1144 | A1144D | 1.17 |
| 1144 | A1144N | 1.38 |
| 1144 | A1144P | 1.42 |
| 1144 | A1144S | 1.53 |
| 1144 | A1144T | 1.24 |
| 1144 | A1144V | 1.34 |
| 1144 | A1144W | 1.33 |
| 1145 | D1145E | 1.64 |
| 1145 | D1145R | 1.58 |
| 1145 | D1145T | 1.68 |
| 1146 | G1146A | 1.16 |
| 1146 | G1146K | 1.15 |
| 1146 | G1146R | 1.13 |
| 1146 | G1146V | 1.21 |
| 1148 | N1148H | 1.16 |
| 1148 | N1148I | 1.13 |
| 1148 | N1148P | 1.33 |
| 1148 | N1148Q | 1.16 |
| 1148 | N1148R | 1.16 |
| 1148 | N1148S | 1.18 |
| 1148 | N1148T | 1.11 |
| 1149 | W1149C | 1.35 |
| 1149 | W1149G | 1.28 |
| 1149 | W1149K | 1.17 |
| 1149 | W1149S | 1.27 |
| 1149 | W1149V | 1.39 |
| 1150 | T1150K | 1.58 |
| 1150 | T1150P | 1.11 |
| 1151 | D1151C | 1.17 |
| 1151 | D1151W | 1.53 |
| 1152 | L1152A | 1.20 |
| 1152 | L1152C | 1.10 |
| 1152 | L1152E | 1.21 |
| 1152 | L1152Q | 1.28 |
| 1152 | L1152W | 1.34 |
| 1153 | A1153E | 1.12 |
| 1153 | A1153G | 1.34 |
| 1153 | A1153L | 1.12 |
| 1154 | A1154D | 1.15 |
| 1154 | A1154E | 1.19 |
| 1154 | A1154R | 1.23 |
| 1158 | I1158S | 1.13 |
| 1158 | I1158W | 1.26 |
| 1159 | A1159E | 1.10 |
| 1159 | A1159I | 1.27 |
| 1161 | Q1161P | 1.24 |
| 1161 | Q1161S | 1.20 |
| 1162 | E1162A | 1.31 |
| 1162 | E1162C | 1.29 |
| 1162 | E1162Q | 1.23 |
| 1162 | E1162W | 1.12 |
| 1165 | E1165M | 1.19 |
| 1165 | E1165S | 1.21 |
| 1166 | R1166Q | 1.15 |
| 1167 | V1167A | 1.11 |
| 1167 | V1167R | 1.67 |
| 1168 | K1168L | 1.13 |
| 1168 | K1168R | 1.13 |
| 1169 | P1169M | 1.16 |
| 1169 | P1169S | 1.26 |
| 1170 | Y1170K | 1.22 |
| 1170 | Y1170M | 1.12 |
| 1170 | Y1170V | 1.16 |
| 1172 | Y1172H | 1.15 |
| 1172 | Y1172S | 1.11 |
| 1173 | D1173G | 1.16 |
| 1173 | D1173T | 1.18 |
| 1174 | F1174D | 1.13 |
| 1175 | A1175S | 1.26 |
| 1178 | G1178M | 1.85 |
| 1178 | G1178Q | 1.27 |
| 1178 | G1178S | 1.18 |
| 1178 | G1178T | 1.33 |
| 1179 | A1179L | 1.37 |
| 1179 | A1179Q | 1.17 |
| 1179 | A1179W | 2.03 |
| 1180 | T1180A | 1.45 |
| 1180 | T1180G | 1.53 |
| 1180 | T1180I | 1.56 |
| 1180 | T1180L | 1.15 |
| 1180 | T1180M | 1.56 |
| 1180 | T1180Q | 1.66 |
| 1180 | T1180S | 1.18 |
| 1180 | T1180Y | 1.41 |
| 1181 | F1181E | 1.19 |
| 1181 | F1181V | 1.12 |
| 1182 | V1182M | 1.26 |
| 1183 | K1183A | 1.14 |
| 1183 | K1183E | 1.22 |
| 1183 | K1183T | 1.16 |
| 1183 | K1183V | 1.12 |
| 1184 | V1184C | 1.28 |
| 1184 | V1184E | 1.87 |
| 1184 | V1184K | 1.28 |
| 1184 | V1184L | 1.73 |
| 1184 | V1184P | 1.65 |
| 1184 | V1184Q | 1.34 |
| 1184 | V1184Y | 1.32 |
| 1185 | T1185Q | 1.19 |
| 1185 | T1185V | 1.10 |
| 1186 | V1186S | 1.41 |
| 1188 | N1188V | 1.13 |
| 1188 | N1188W | 1.45 |
| 1189 | A1189C | 1.30 |
| 1189 | A1189K | 1.27 |
| 1189 | A1189T | 1.28 |
| 1189 | A1189V | 1.20 |
| 1190 | D1190R | 1.22 |
| 1190 | D1190T | 1.39 |
| 1190 | D1190Y | 1.11 |
| 1191 | T1191L | 1.32 |
| 1193 | T1193G | 1.10 |
| 1194 | P1194E | 1.11 |
| 1194 | P1194W | 1.14 |
| 1197 | V1197A | 1.35 |
| 1199 | C1199D | 1.17 |
| 1200 | A1200G | 1.20 |
| 1203 | T1203K | 1.25 |
| 1204 | E1204G | 1.39 |
| 1204 | E1204S | 1.11 |
| 1205 | I1205V | 1.21 |
| 1208 | K1208R | 1.23 |
| 1208 | K1208V | 1.29 |
| 1208 | K1208W | 1.57 |
| 1209 | T1209A | 1.25 |
| 1209 | T1209C | 1.27 |
| 1209 | T1209E | 1.48 |
| 1209 | T1209K | 1.45 |
| 1209 | T1209N | 1.21 |
| 1209 | T1209Q | 1.20 |
| 1209 | T1209R | 1.18 |
| 1209 | T1209W | 1.16 |
| 1210 | A1210D | 1.59 |
| 1210 | A1210G | 1.73 |
| 1210 | A1210K | 1.39 |
| 1210 | A1210L | 1.34 |
| 1210 | A1210Q | 1.14 |
| 1210 | A1210R | 1.53 |
| 1210 | A1210T | 1.94 |
| 1210 | A1210W | 1.40 |
| 1211 | T1211C | 1.12 |
| 1211 | T1211D | 1.24 |
| 1211 | T1211E | 1.18 |
| 1211 | T1211G | 1.84 |
| 1211 | T1211H | 1.11 |
| 1211 | T1211K | 1.35 |
| 1211 | T1211P | 1.92 |
| 1211 | T1211Q | 1.16 |
| 1211 | T1211R | 1.44 |
| 1211 | T1211S | 1.30 |
| 1211 | T1211V | 1.18 |
| 1213 | K1213A | 1.13 |
| 1213 | K1213D | 1.12 |
| 1213 | K1213S | 1.14 |
| 1213 | K1213T | 1.22 |
| 1214 | F1214A | 1.54 |
| 1214 | F1214E | 1.25 |
| 1214 | F1214K | 1.73 |
| 1214 | F1214L | 1.25 |
| 1214 | F1214P | 1.27 |
| 1214 | F1214S | 1.31 |
| 1214 | F1214V | 1.65 |
| 1215 | V1215D | 1.44 |
| 1215 | V1215E | 1.12 |
| 1215 | V1215L | 1.36 |
| 1215 | V1215Q | 1.23 |
| 1215 | V1215W | 1.13 |
| 1216 | T1216A | 1.24 |
| 1216 | T1216L | 1.55 |
| 1216 | T1216P | 1.28 |
| 1216 | T1216Q | 1.75 |
| 1216 | T1216R | 1.13 |
| 1217 | N1217A | 1.42 |
| 1217 | N1217E | 1.14 |
| 1217 | N1217P | 1.28 |
| 1217 | N1217R | 1.22 |
| 1217 | N1217S | 1.31 |
| 1217 | N1217T | 1.32 |
| 1218 | T1218A | 1.27 |
| 1218 | T1218C | 1.34 |
| 1218 | T1218E | 1.40 |
| 1218 | T1218G | 1.20 |
| 1218 | T1218Q | 1.10 |
| 1218 | T1218S | 1.31 |
| 1218 | T1218V | 1.16 |
| 1218 | T1218W | 1.45 |
| 1219 | S1219I | 1.21 |
| 1219 | S1219K | 1.20 |
| 1219 | S1219R | 1.26 |
| 1219 | S1219V | 1.10 |
| 1220 | A1220C | 1.19 |
| 1220 | A1220G | 1.20 |
| 1220 | A1220P | 1.26 |
| 1221 | A1221K | 1.19 |
| 1221 | A1221R | 1.21 |
| 1221 | A1221V | 1.20 |
| 1221 | A1221W | 1.24 |
| 1222 | L1222A | 1.34 |
| 1222 | L1222E | 1.38 |
| 1222 | L1222F | 1.43 |
| 1222 | L1222Q | 1.17 |
| 1222 | L1222R | 1.17 |
| 1222 | L1222V | 1.71 |
| 1222 | L1222W | 1.27 |
| 1224 | S1224P | 1.51 |
| 1224 | S1224W | 1.47 |
| 1225 | L1225E | 1.66 |
| 1225 | L1225K | 1.84 |
| 1225 | L1225V | 1.18 |
| 1226 | T1226P | 1.76 |
| 1227 | V1227C | 1.10 |
| 1227 | V1227D | 1.28 |
| 1227 | V1227P | 1.29 |
| 1227 | V1227Q | 1.14 |
| 1229 | G1229C | 1.15 |
| 1229 | G1229E | 1.13 |
| 1229 | G1229V | 1.57 |
| 1230 | T1230H | 1.22 |
| 1230 | T1230K | 1.47 |
| 1230 | T1230W | 1.73 |
| 1231 | K1231G | 1.18 |
| 1232 | V1232E | 1.11 |
| 1232 | V1232Q | 1.15 |
| 1232 | V1232R | 1.39 |
| 1234 | D1234V | 1.19 |
| 1235 | S1235D | 1.20 |
| 1235 | S1235E | 1.23 |
| 1235 | S1235G | 1.27 |
| 1235 | S1235P | 1.12 |
| 1235 | S1235R | 1.20 |
| 1235 | S1235W | 1.31 |
| 1235 | S1235Y | 1.12 |
| 1236 | V1236A | 1.32 |
| 1236 | V1236C | 1.11 |
| 1236 | V1236G | 1.35 |
| 1236 | V1236P | 1.34 |
| 1236 | V1236Q | 1.21 |
| 1237 | L1237V | 1.14 |
| 1238 | A1238D | 1.10 |
| 1238 | A1238E | 1.95 |
| 1238 | A1238L | 1.22 |
| 1238 | A1238R | 1.41 |
| 1239 | A1239R | 1.22 |
| 1240 | G1240D | 1.20 |
| 1240 | G1240Q | 1.13 |
| 1243 | N1243C | 1.20 |
| 1247 | I1247R | 1.28 |
| 1255 | G1255H | 1.30 |
| 1259 | A1259K | 1.58 |
| 1261 | V1261R | 1.15 |
| 1262 | T1262F | 1.19 |
| 1262 | T1262M | 1.11 |
| 1262 | T1262R | 1.33 |
| 1263 | V1263T | 1.40 |
| 1264 | L1264H | 1.22 |
| 1264 | L1264R | 1.12 |
| 1265 | P1265W | 1.93 |
| 1273 | V1273R | 1.26 |
| 1274 | I1274F | 1.91 |
| 1274 | I1274R | 1.63 |
| 1275 | T1275A | 1.11 |
| 1275 | T1275W | 1.98 |
| 1276 | E1276R | 1.12 |
| 1277 | S1277L | 1.36 |
| 1277 | S1277T | 1.32 |
| 1277 | S1277W | 1.97 |
| 1278 | E1278Q | 1.78 |
| 1278 | E1278R | 1.20 |
| 1279 | D1279I | 1.25 |
| 1279 | D1279R | 1.63 |
| 1279 | D1279W | 1.78 |
| 1280 | H1280C | 1.19 |
| 1280 | H1280E | 1.48 |
| 1280 | H1280G | 1.25 |
| 1281 | V1281I | 1.74 |
| 1281 | V1281W | 1.52 |
| 1284 | K1284G | 2.25 |
| 1285 | T1285A | 1.16 |
| 1285 | T1285M | 1.11 |
| 1286 | F1286A | 1.27 |
| 1286 | F1286E | 1.60 |
| 1286 | F1286P | 1.32 |
| 1286 | F1286R | 1.11 |
| 1287 | T1287C | 3.44 |
| 1287 | T1287L | 1.15 |
| 1287 | T1287S | 1.31 |
| 1287 | T1287W | 1.76 |
| 1288 | U288A | 1.85 |
| 1288 | I1288D | 2.34 |
| 1288 | I1288G | 1.52 |
| 1288 | I1288K | 1.60 |
| 1293 | E1293K | 1.27 |
| 1293 | E1293L | 1.22 |
| 1294 | Q1294E | 1.25 |
| 1295 | E1295K | 1.36 |
| 1296 | F1296A | 1.37 |
| 1302 | E1302G | 1.15 |
| 1302 | E1302R | 2.50 |
| 1302 | E1302S | 2.76 |
| 1303 | R1303S | 1.27 |
| 1304 | D1304V | 1.51 |

TABLE 2
Variants having a GI-IF of at most 0.9

| Position | Mutation | GI-IF |
|---|---|---|
| 3 | D3H | 0.9 |
| 3 | D3S | 0.55 |
| 3 | D3W | 0.62 |
| 4 | A4C | 0.85 |
| 4 | A4G | 0.85 |
| 4 | A4I | 0.8 |
| 4 | A4L | 0.8 |
| 4 | A4M | 0.9 |
| 4 | A4P | 0.85 |
| 5 | T5D | 0.78 |
| 5 | T5F | 0.89 |
| 5 | T5K | 0.86 |
| 5 | T5R | 0.89 |
| 5 | T5V | 0.81 |
| 6 | R6A | 0.76 |
| 6 | R6D | 0.82 |
| 6 | R6F | 0.73 |
| 6 | R6M | 0.68 |
| 6 | R6P | 0.65 |
| 6 | R6S | 0.89 |
| 7 | S7A | 0.82 |
| 7 | S7D | 0.69 |
| 7 | S7I | 0.79 |
| 7 | S7W | 0.86 |
| 9 | S9P | 0.76 |
| 9 | S9R | 0.89 |
| 10 | T10G | 0.84 |
| 10 | T10P | 0.71 |
| 10 | T10R | 0.89 |
| 10 | T10W | 0.86 |
| 11 | T11L | 0.78 |
| 11 | T11P | 0.76 |
| 12 | Q12L | 0.88 |
| 12 | Q12R | 0.76 |
| 12 | Q12Y | 0.88 |
| 13 | M13F | 0.65 |
| 14 | S14A | 0.83 |
| 14 | S14L | 0.84 |
| 15 | S15Y | 0.42 |
| 16 | T16A | 0.86 |
| 16 | T16N | 0.83 |
| 16 | T16S | 0.8 |
| 17 | P17C | 0.84 |
| 17 | P17D | 0.64 |
| 17 | P17E | 0.86 |
| 17 | P17L | 0.65 |
| 17 | P17N | 0.81 |
| 17 | P17R | 0.9 |
| 17 | P17T | 0.74 |
| 19 | V19G | 0.64 |
| 19 | V19W | 0.82 |
| 20 | V20K | 0.74 |
| 20 | V20N | 0.89 |
| 20 | V20W | 0.79 |
| 21 | Y21F | 0.88 |
| 22 | S22E | 0.77 |
| 22 | S22F | 0.89 |
| 22 | S22G | 0.74 |
| 22 | S22L | 0.82 |
| 22 | S22M | 0.84 |
| 23 | S23C | 0.8 |
| 23 | S23D | 0.86 |
| 23 | S23L | 0.56 |
| 23 | S23M | 0.58 |
| 23 | S23R | 0.74 |
| 24 | A24G | 0.89 |
| 25 | V25H | 0.86 |
| 25 | V25K | 0.68 |
| 25 | V25L | 0.88 |
| 25 | V25R | 0.88 |
| 26 | D26T | 0.9 |
| 27 | S27F | 0.8 |
| 29 | Q29D | 0.84 |
| 29 | Q29V | 0.62 |
| 29 | Q29W | 0.78 |
| 30 | N30A | 0.72 |
| 30 | N30G | 0.89 |
| 30 | N30H | 0.89 |
| 30 | N30P | 0.85 |
| 31 | R31G | 0.74 |
| 31 | R31I | 0.72 |
| 32 | T32M | 0.88 |
| 32 | T32R | 0.81 |
| 33 | S33C | 0.64 |
| 33 | S33E | 0.85 |
| 33 | S33H | 0.9 |
| 33 | S33N | 0.77 |
| 33 | S33Q | 0.66 |
| 33 | S33R | 0.66 |
| 33 | S33V | 0.88 |
| 34 | D34C | 0.73 |
| 34 | D34E | 0.79 |
| 34 | D34L | 0.76 |
| 34 | D34S | 0.55 |
| 34 | D34W | 0.78 |
| 34 | D34Y | 0.67 |
| 35 | F35E | 0.59 |
| 35 | F35K | 0.75 |
| 35 | F35N | 0.69 |
| 35 | F35T | 0.85 |
| 36 | D36H | 0.89 |
| 37 | A37Q | 0.81 |
| 38 | N38C | 0.84 |
| 38 | N38G | 0.83 |
| 40 | K40D | 0.74 |
| 40 | K40N | 0.78 |
| 41 | F41G | 0.64 |
| 41 | F41I | 0.78 |
| 41 | F41Q | 0.73 |
| 41 | F41S | 0.88 |
| 41 | F41Y | 0.81 |
| 42 | M42E | 0.6 |
| 42 | M42N | 0.79 |
| 43 | L43C | 0.64 |
| 43 | L43T | 0.58 |
| 43 | L43V | 0.69 |
| 44 | S44C | 0.76 |
| 44 | S44N | 0.83 |
| 44 | S44Y | 0.82 |
| 45 | D45A | 0.7 |
| 48 | Q48S | 0.9 |
| 49 | A49R | 0.31 |
| 49 | A49S | 0.74 |
| 49 | A49V | 0.78 |
| 51 | D51P | 0.57 |
| 51 | D51V | 0.69 |
| 53 | A53C | 0.79 |
| 53 | A53L | 0.84 |
| 53 | A53R | 0.77 |
| 53 | A53S | 0.6 |
| 53 | A53T | 0.83 |
| 53 | A53V | 0.71 |
| 53 | A53W | 0.74 |
| 54 | F54M | 0.81 |
| 54 | F54S | 0.8 |
| 58 | A58D | 0.73 |
| 58 | A58G | 0.65 |
| 58 | A58M | 0.84 |
| 60 | Q60A | 0.89 |
| 60 | Q60C | 0.82 |
| 60 | Q60K | 0.61 |
| 61 | Q61K | 0.87 |
| 61 | Q61S | 0.85 |
| 62 | V62S | 0.68 |
| 63 | D63P | 0.84 |
| 63 | D63V | 0.81 |
| 66 | H66C | 0.9 |
| 66 | H66L | 0.74 |
| 66 | H66W | 0.85 |
| 66 | H66Y | 0.44 |
| 67 | D67E | 0.81 |
| 68 | Y68P | 0.68 |
| 68 | Y68V | 0.84 |
| 70 | I70A | 0.74 |
| 71 | T71C | 0.71 |
| 71 | T71E | 0.82 |
| 71 | T71G | 0.8 |
| 71 | T71K | 0.77 |
| 71 | T71N | 0.88 |
| 71 | T71Q | 0.79 |
| 73 | K73F | 0.82 |
| 74 | Y74T | 0.85 |
| 74 | Y74W | 0.8 |
| 76 | Q76K | 0.74 |
| 76 | Q76M | 0.87 |
| 76 | Q76S | 0.83 |
| 76 | Q76V | 0.79 |
| 76 | Q76Y | 0.9 |
| 77 | S77F | 0.5 |
| 77 | S77Y | 0.85 |
| 78 | N78E | 0.67 |
| 78 | N78K | 0.71 |
| 78 | N78Q | 0.78 |
| 78 | N78R | 0.74 |
| 78 | N78T | 0.86 |
| 79 | E79Q | 0.7 |
| 79 | E79W | 0.84 |
| 80 | A80K | 0.78 |
| 81 | E81A | 0.56 |
| 81 | E81Q | 0.44 |
| 85 | L85M | 0.84 |
| 86 | P86H | 0.86 |
| 86 | P86N | 0.74 |
| 87 | G87A | 0.84 |
| 87 | G87E | 0.81 |
| 87 | G87Q | 0.73 |
| 88 | G88I | 0.9 |
| 88 | G88Q | 0.6 |
| 88 | G88S | 0.76 |
| 89 | T89G | 0.84 |
| 89 | T89H | 0.79 |
| 89 | T89K | 0.8 |
| 89 | T89P | 0.89 |
| 90 | G90C | 0.88 |
| 90 | G90V | 0.71 |
| 91 | W91R | 0.78 |
| 91 | W91S | 0.81 |
| 96 | F96S | 0.28 |
| 106 | R106P | 0.83 |
| 107 | I107G | 0.7 |
| 107 | I107Q | 0.88 |
| 108 | A108S | 0.76 |
| 112 | D112F | 0.71 |
| 112 | D112G | 0.84 |
| 112 | D112T | 0.24 |
| 113 | G113A | 0.88 |
| 113 | G113S | 0.8 |
| 114 | V114G | 0.83 |
| 114 | V114M | 0.8 |
| 116 | M116Y | 0.86 |
| 117 | N117R | 0.89 |
| 117 | N117T | 0.89 |
| 117 | N117W | 0.76 |
| 118 | A118K | 0.66 |
| 118 | A118Y | 0.66 |
| 119 | T119A | 0.71 |
| 119 | T119L | 0.82 |
| 121 | W121Y | 0.7 |
| 122 | F122A | 0.77 |
| 122 | F122M | 0.86 |
| 122 | F122S | 0.69 |
| 122 | F122Y | 0.69 |
| 123 | N123P | 0.68 |
| 124 | G124E | 0.45 |
| 124 | G124Q | 0.73 |
| 124 | G124R | 0.79 |
| 125 | V125E | 0.79 |
| 125 | V125I | 0.77 |
| 128 | G128A | 0.89 |
| 128 | G128D | 0.79 |
| 129 | T129E | 0.73 |
| 130 | H130Q | 0.88 |
| 131 | P131L | 0.89 |
| 131 | P131S | 0.85 |
| 132 | Y132C | 0.66 |
| 132 | Y132S | 0.87 |
| 133 | G133E | 0.88 |
| 135 | S135E | 0.86 |
| 135 | S135P | 0.81 |
| 135 | S135V | 0.85 |
| 136 | P136R | 0.51 |
| 136 | P136Y | 0.78 |
| 137 | F137A | 0.9 |
| 137 | F137G | 0.81 |
| 138 | S138G | 0.86 |
| 138 | S138L | 0.73 |
| 138 | S138R | 0.73 |
| 138 | S138V | 0.89 |
| 139 | F139A | 0.84 |
| 139 | F139E | 0.9 |
| 139 | F139Q | 0.78 |
| 139 | F139W | 0.81 |
| 140 | D140G | 0.83 |
| 140 | D140L | 0.81 |
| 141 | L141T | 0.87 |
| 142 | T142E | 0.85 |
| 142 | T142V | 0.79 |
| 146 | K146A | 0.56 |
| 148 | G148H | 0.9 |
| 148 | G148T | 0.74 |
| 149 | G149H | 0.89 |
| 149 | G149Q | 0.8 |
| 153 | H53A | 0.49 |
| 153 | I153Y | 0.81 |
| 154 | V154S | 0.89 |
| 155 | V155F | 0.74 |
| 157 | V157L | 0.69 |
| 158 | E158G | 0.56 |
| 159 | N159S | 0.77 |
| 168 | S168C | 0.84 |
| 169 | G169C | 0.84 |
| 169 | G169D | 0.67 |
| 170 | S170L | 0.73 |
| 171 | G171T | 0.77 |
| 173 | Y173W | 0.72 |
| 175 | D175E | 0.83 |
| 177 | T177A | 0.84 |
| 177 | T177L | 0.81 |
| 181 | T181D | 0.83 |
| 181 | T181K | 0.88 |
| 181 | T181R | 0.87 |
| 182 | D182F | 0.73 |
| 182 | D182L | 0.88 |
| 182 | D182S | 0.8 |
| 188 | N188E | 0.7 |
| 189 | N189E | 0.46 |
| 190 | G190C | 0.52 |
| 190 | G190H | 0.88 |
| 190 | G190Q | 0.88 |
| 191 | V191C | 0.84 |
| 191 | V191W | 0.85 |
| 194 | K194A | 0.82 |
| 194 | K194L | 0.82 |
| 194 | K194R | 0.83 |
| 195 | T195A | 0.88 |
| 195 | T195E | 0.84 |
| 195 | T195S | 0.69 |
| 196 | P196H | 0.83 |
| 197 | S197A | 0.88 |
| 197 | S197K | 0.85 |
| 197 | S197P | 0.83 |
| 198 | L198E | 0.82 |
| 198 | L198F | 0.83 |
| 198 | L198H | 0.8 |
| 198 | L198K | 0.8 |
| 198 | L198R | 0.67 |
| 198 | L198V | 0.72 |
| 198 | L198W | 0.85 |
| 199 | A199F | 0.87 |
| 199 | A199K | 0.72 |
| 199 | A199R | 0.87 |
| 201 | Q201C | 0.86 |
| 201 | Q201I | 0.81 |
| 201 | Q201M | 0.87 |
| 201 | Q201V | 0.87 |
| 202 | N202D | 0.71 |
| 202 | N202F | 0.89 |
| 202 | N202G | 0.89 |
| 202 | N202K | 0.88 |
| 202 | N202L | 0.78 |
| 202 | N202R | 0.64 |
| 202 | N202T | 0.79 |
| 203 | G203C | 0.79 |
| 203 | G203K | 0.7 |
| 203 | G203M | 0.87 |
| 203 | G203R | 0.9 |
| 203 | G203S | 0.77 |
| 203 | G203V | 0.65 |
| 203 | G203W | 0.75 |
| 203 | G203Y | 0.79 |
| 204 | G204A | 0.75 |
| 204 | G204C | 0.84 |
| 204 | G204D | 0.8 |
| 204 | G204K | 0.71 |
| 204 | G204S | 0.88 |
| 204 | G204Y | 0.66 |
| 205 | N205E | 0.68 |
| 205 | N205G | 0.65 |
| 205 | N205H | 0.68 |
| 205 | N205L | 0.8 |
| 205 | N205P | 0.83 |
| 205 | N205Y | 0.86 |
| 206 | V206A | 0.88 |
| 206 | V206D | 0.88 |
| 206 | V206F | 0.86 |
| 206 | V206G | 0.82 |
| 206 | V206I | 0.75 |
| 206 | V206K | 0.67 |
| 206 | V206Q | 0.86 |
| 206 | V206R | 0.81 |
| 206 | V206S | 0.79 |
| 206 | V206T | 0.81 |
| 207 | T207A | 0.78 |
| 207 | T207C | 0.77 |
| 207 | T207G | 0.71 |
| 207 | T207I | 0.75 |
| 207 | T207K | 0.73 |
| 207 | T207M | 0.88 |
| 207 | T207N | 0.82 |
| 207 | T207Q | 0.63 |
| 207 | T207R | 0.8 |
| 207 | T207W | 0.84 |
| 208 | M208A | 0.88 |
| 208 | M208S | 0.74 |
| 208 | M208T | 0.83 |
| 209 | N209C | 0.83 |
| 209 | N209D | 0.7 |
| 209 | N209G | 0.88 |
| 209 | N209K | 0.7 |
| 209 | N209L | 0.83 |
| 209 | N209Q | 0.87 |
| 209 | N209R | 0.9 |
| 209 | N209V | 0.71 |
| 210 | L210A | 0.89 |
| 210 | L210C | 0.81 |
| 210 | L210F | 0.86 |
| 210 | L210G | 0.77 |
| 210 | L210H | 0.87 |
| 210 | L210I | 0.78 |
| 210 | L210Q | 0.76 |
| 210 | L210R | 0.81 |
| 210 | L210S | 0.85 |
| 210 | L210T | 0.83 |
| 210 | L210V | 0.75 |
| 211 | T211A | 0.77 |
| 211 | T211D | 0.86 |
| 211 | T211E | 0.83 |
| 211 | T211F | 0.79 |
| 211 | T211K | 0.88 |
| 211 | T211N | 0.89 |
| 211 | T211Q | 0.77 |
| 211 | T211R | 0.63 |
| 211 | T211S | 0.8 |
| 212 | T212A | 0.86 |
| 212 | T212C | 0.88 |
| 212 | T212E | 0.64 |
| 212 | T212F | 0.87 |
| 212 | T212G | 0.76 |
| 212 | T212H | 0.72 |
| 212 | T212K | 0.87 |
| 212 | T212L | 0.77 |
| 212 | T212M | 0.88 |
| 212 | T212S | 0.74 |
| 212 | T212W | 0.8 |
| 213 | K213C | 0.66 |
| 213 | K213D | 0.66 |
| 213 | K213F | 0.74 |
| 213 | K213I | 0.8 |
| 213 | K213L | 0.79 |
| 213 | K213M | 0.8 |
| 213 | K213N | 0.8 |
| 213 | K213Q | 0.85 |
| 213 | K213R | 0.84 |
| 213 | K213S | 0.81 |
| 213 | K213T | 0.7 |
| 213 | K213V | 0.82 |
| 213 | K213Y | 0.76 |
| 214 | V214A | 0.66 |
| 214 | V214C | 0.77 |
| 214 | V214T | 0.84 |
| 214 | V214W | 0.74 |
| 215 | A215E | 0.75 |
| 215 | A215F | 0.9 |
| 215 | A215I | 0.77 |
| 215 | A215K | 0.76 |
| 215 | A215L | 0.79 |
| 215 | A215Q | 0.64 |
| 215 | A215R | 0.74 |
| 215 | A215S | 0.88 |
| 215 | A215V | 0.79 |
| 216 | N216T | 0.87 |
| 216 | N216V | 0.81 |
| 217 | D217T | 0.75 |
| 217 | D217V | 0.83 |
| 219 | K219C | 0.87 |
| 219 | K219F | 0.8 |
| 219 | K219H | 0.76 |
| 219 | K219M | 0.74 |
| 220 | A220C | 0.74 |
| 220 | A220I | 0.81 |
| 220 | A220L | 0.79 |
| 220 | A220M | 0.81 |
| 220 | A220T | 0.82 |
| 220 | A220V | 0.53 |
| 220 | A220W | 0.86 |
| 221 | A221E | 0.68 |
| 221 | A221L | 0.76 |
| 221 | A221N | 0.82 |
| 221 | A221R | 0.78 |
| 221 | A221V | 0.9 |
| 221 | A221Y | 0.8 |
| 222 | A222D | 0.74 |
| 222 | A222I | 0.8 |
| 222 | A222L | 0.8 |
| 222 | A222P | 0.72 |
| 222 | A222W | 0.75 |
| 222 | A222Y | 0.8 |
| 223 | N223A | 0.82 |
| 223 | N223E | 0.76 |
| 223 | N223F | 0.79 |
| 223 | N223G | 0.84 |
| 223 | N223K | 0.7 |
| 223 | N223L | 0.76 |
| 223 | N223M | 0.86 |
| 223 | N223R | 0.84 |
| 223 | N223T | 0.77 |
| 223 | N223V | 0.85 |
| 223 | N223W | 0.84 |
| 224 | I224Q | 0.84 |
| 225 | T225A | 0.9 |
| 225 | T225G | 0.86 |
| 226 | L226C | 0.88 |
| 226 | L226M | 0.8 |
| 227 | K227T | 0.84 |
| 229 | T229A | 0.7 |
| 229 | T229C | 0.77 |
| 229 | T229D | 0.73 |
| 229 | T229G | 0.7 |
| 229 | T229H | 0.9 |
| 229 | T229M | 0.75 |
| 229 | T229N | 0.75 |
| 229 | T229Q | 0.75 |
| 229 | T229R | 0.74 |
| 229 | T229V | 0.68 |
| 230 | V230F | 0.82 |
| 230 | V230L | 0.74 |
| 230 | V230M | 0.61 |
| 230 | V230Q | 0.81 |
| 230 | V230R | 0.77 |
| 230 | V230S | 0.71 |
| 231 | F231A | 0.9 |
| 231 | F231E | 0.71 |
| 231 | F231G | 0.66 |
| 231 | F231I | 0.78 |
| 231 | F231K | 0.82 |
| 231 | F231Q | 0.85 |
| 231 | F231S | 0.81 |
| 231 | F231V | 0.7 |
| 231 | F231W | 0.79 |
| 231 | F231Y | 0.69 |
| 232 | P232G | 0.8 |
| 232 | P232H | 0.87 |
| 232 | P232L | 0.8 |
| 232 | P232M | 0.71 |
| 232 | P232R | 0.8 |
| 232 | P232S | 0.65 |
| 232 | P232V | 0.85 |
| 232 | P232W | 0.86 |
| 232 | P232Y | 0.82 |
| 233 | K233A | 0.84 |
| 233 | K233C | 0.66 |
| 233 | K233E | 0.72 |
| 233 | K233F | 0.75 |
| 233 | K233G | 0.87 |
| 233 | K233L | 0.69 |
| 233 | K233P | 0.59 |
| 233 | K233R | 0.8 |
| 233 | K233S | 0.89 |
| 233 | K233V | 0.8 |
| 233 | K233W | 0.88 |
| 233 | K233Y | 0.82 |
| 234 | G234A | 0.79 |
| 234 | G234C | 0.7 |
| 234 | G234D | 0.71 |
| 234 | G234E | 0.76 |
| 234 | G234K | 0.89 |
| 234 | G234L | 0.65 |
| 234 | G234Q | 0.78 |
| 234 | G234R | 0.78 |
| 234 | G234W | 0.85 |
| 234 | G234Y | 0.68 |
| 235 | G235C | 0.84 |
| 235 | G235F | 0.81 |
| 235 | G235H | 0.7 |
| 235 | G235I | 0.76 |
| 235 | G235M | 0.64 |
| 235 | G235Q | 0.76 |
| 235 | G235R | 0.9 |
| 235 | G235T | 0.75 |
| 235 | G235W | 0.78 |
| 235 | G235Y | 0.67 |
| 236 | K236A | 0.78 |
| 236 | K236D | 0.63 |
| 236 | K236E | 0.88 |
| 236 | K236G | 0.84 |
| 236 | K236L | 0.65 |
| 236 | K236R | 0.73 |
| 236 | K236S | 0.75 |
| 236 | K236T | 0.84 |
| 236 | K236W | 0.66 |
| 236 | K236Y | 0.62 |
| 237 | T237F | 0.81 |
| 237 | T237I | 0.84 |
| 237 | T237K | 0.77 |
| 237 | T237M | 0.82 |
| 237 | T237Q | 0.88 |
| 237 | T237R | 0.78 |
| 237 | T237S | 0.88 |
| 237 | T237V | 0.89 |
| 237 | T237Y | 0.88 |
| 238 | D238A | 0.79 |
| 238 | D238E | 0.63 |
| 238 | D238F | 0.6 |
| 238 | D238G | 0.73 |
| 238 | D238H | 0.84 |
| 238 | D238I | 0.82 |
| 238 | D238L | 0.83 |
| 238 | D238M | 0.76 |
| 238 | D238N | 0.9 |
| 238 | D238P | 0.7 |
| 238 | D238Q | 0.81 |
| 238 | D238R | 0.73 |
| 239 | A239C | 0.84 |
| 239 | A239E | 0.72 |
| 239 | A239G | 0.29 |
| 239 | A239I | 0.8 |
| 239 | A239K | 0.85 |
| 239 | A239T | 0.48 |
| 240 | A240C | 0.63 |
| 240 | A240E | 0.85 |
| 240 | A240L | 0.72 |
| 240 | A240P | 0.71 |
| 240 | A240T | 0.79 |
| 240 | A240V | 0.81 |
| 240 | A240W | 0.9 |
| 240 | A240Y | 0.78 |
| 241 | I241T | 0.71 |
| 242 | G242T | 0.83 |
| 242 | G242Y | 0.52 |
| 243 | T243M | 0.64 |
| 243 | T243R | 0.78 |
| 243 | T243V | 0.83 |
| 244 | V244A | 0.85 |
| 244 | V244E | 0.73 |
| 244 | V244G | 0.84 |
| 244 | V244L | 0.83 |
| 244 | V244R | 0.76 |
| 245 | T245E | 0.79 |
| 245 | T245G | 0.67 |
| 245 | T245L | 0.83 |
| 245 | T245M | 0.84 |
| 245 | T245N | 0.85 |
| 245 | T245Q | 0.64 |
| 245 | T245R | 0.77 |
| 245 | T245S | 0.81 |
| 246 | T246D | 0.89 |
| 246 | T246E | 0.86 |
| 246 | T246G | 0.77 |
| 246 | T246K | 0.79 |
| 246 | T246V | 0.9 |
| 247 | A247D | 0.87 |
| 247 | A247E | 0.74 |
| 247 | A247N | 0.83 |
| 247 | A247P | 0.59 |
| 247 | A247Q | 0.84 |
| 247 | A247R | 0.49 |
| 247 | A247S | 0.74 |
| 247 | A247V | 0.7 |
| 247 | A247W | 0.65 |
| 248 | S248A | 0.77 |
| 248 | S248E | 0.58 |
| 248 | S248H | 0.88 |
| 248 | S248I | 0.89 |
| 248 | S248L | 0.79 |
| 248 | S248Q | 0.78 |
| 248 | S248T | 0.68 |
| 248 | S248Y | 0.88 |
| 249 | K249A | 0.77 |
| 249 | K249D | 0.71 |
| 249 | K249G | 0.53 |
| 249 | K249H | 0.76 |
| 249 | K249I | 0.86 |
| 249 | K249L | 0.76 |
| 249 | K249M | 0.84 |
| 249 | K249N | 0.66 |
| 249 | K249P | 0.78 |
| 249 | K249Q | 0.73 |
| 249 | K249S | 0.85 |
| 249 | K249T | 0.9 |
| 249 | K249V | 0.74 |
| 249 | K249Y | 0.58 |
| 250 | S250H | 0.73 |
| 250 | S250M | 0.88 |
| 250 | S250W | 0.89 |
| 251 | I251L | 0.77 |
| 251 | I251V | 0.81 |
| 252 | A252C | 0.81 |
| 252 | A252E | 0.83 |
| 252 | A252F | 0.58 |
| 252 | A252I | 0.8 |
| 252 | A252R | 0.88 |
| 252 | A252S | 0.83 |
| 254 | G254D | 0.75 |
| 254 | G254F | 0.75 |
| 254 | G254I | 0.47 |
| 254 | G254L | 0.74 |
| 254 | G254M | 0.69 |
| 254 | G254Q | 0.77 |
| 254 | G254R | 0.57 |
| 254 | G254W | 0.64 |
| 255 | A255C | 0.87 |
| 255 | A255F | 0.83 |
| 255 | A255K | 0.59 |
| 255 | A255L | 0.83 |
| 255 | A255M | 0.81 |
| 255 | A255S | 0.69 |
| 255 | A255T | 0.9 |
| 255 | A255W | 0.9 |
| 255 | A255Y | 0.63 |
| 256 | S256A | 0.63 |
| 256 | S256C | 0.78 |
| 256 | S256F | 0.83 |
| 256 | S256G | 0.85 |
| 256 | S256K | 0.69 |
| 256 | S256L | 0.81 |
| 256 | S256M | 0.78 |
| 256 | S256N | 0.67 |
| 256 | S256Q | 0.81 |
| 256 | S256R | 0.77 |
| 256 | S256V | 0.84 |
| 256 | S256W | 0.76 |
| 256 | S256Y | 0.54 |
| 257 | A257I | 0.76 |
| 257 | A257T | 0.82 |
| 258 | D258A | 0.74 |
| 258 | D258L | 0.65 |
| 258 | D258M | 0.88 |
| 258 | D258W | 0.68 |
| 259 | V259E | 0.76 |
| 259 | V259L | 0.84 |
| 259 | V259S | 0.86 |
| 259 | V259T | 0.75 |
| 260 | T260A | 0.87 |
| 260 | T260D | 0.74 |
| 260 | T260G | 0.79 |
| 260 | T260I | 0.9 |
| 260 | T260K | 0.88 |
| 261 | S261D | 0.8 |
| 261 | S261R | 0.52 |
| 261 | S261W | 0.66 |
| 262 | T262D | 0.56 |
| 262 | T262G | 0.81 |
| 262 | T262H | 0.87 |
| 262 | T262L | 0.87 |
| 262 | T262W | 0.77 |
| 263 | I263C | 0.83 |
| 263 | I263G | 0.87 |
| 263 | I263L | 0.86 |
| 263 | I263S | 0.82 |
| 263 | I263V | 0.86 |
| 264 | T264F | 0.78 |
| 264 | T264G | 0.88 |
| 264 | T264K | 0.83 |
| 264 | T264M | 0.45 |
| 264 | T264P | 0.84 |
| 264 | T264Q | 0.74 |
| 264 | T264R | 0.85 |
| 264 | T264S | 0.83 |
| 264 | T264Y | 0.74 |
| 265 | A265G | 0.83 |
| 265 | A265I | 0.77 |
| 265 | A265K | 0.85 |
| 265 | A265R | 0.85 |
| 265 | A265S | 0.75 |
| 265 | A265V | 0.9 |
| 266 | A266E | 0.88 |
| 266 | A266G | 0.76 |
| 266 | A266K | 0.66 |
| 266 | A266L | 0.86 |
| 266 | A266M | 0.85 |
| 266 | A266P | 0.88 |
| 266 | A266Q | 0.68 |
| 266 | A266S | 0.86 |
| 266 | A266T | 0.74 |
| 267 | S267A | 0.75 |
| 267 | S267D | 0.8 |
| 267 | S267K | 0.85 |
| 267 | S267M | 0.8 |
| 267 | S267N | 0.79 |
| 267 | S267P | 0.77 |
| 267 | S267Q | 0.86 |
| 267 | S267R | 0.75 |
| 268 | P268G | 0.76 |
| 268 | P268M | 0.84 |
| 268 | P268V | 0.9 |
| 268 | P268W | 0.7 |
| 268 | P268Y | 0.73 |
| 269 | K269G | 0.87 |
| 269 | K269R | 0.81 |
| 269 | K269V | 0.73 |
| 269 | K269Y | 0.71 |
| 270 | L270D | 0.74 |
| 270 | L270M | 0.84 |
| 270 | L270R | 0.84 |
| 270 | L270V | 0.85 |
| 272 | S272E | 0.88 |
| 272 | S272G | 0.7 |
| 272 | S272K | 0.88 |
| 272 | S272L | 0.83 |
| 272 | S272N | 0.83 |
| 273 | I273K | 0.89 |
| 273 | I273L | 0.8 |
| 273 | I273P | 0.89 |
| 273 | I273R | 0.78 |
| 273 | I273S | 0.8 |
| 274 | K274D | 0.88 |
| 274 | K274P | 0.87 |
| 274 | K274R | 0.84 |
| 275 | N275K | 0.88 |
| 275 | N275V | 0.87 |
| 275 | N275W | 0.75 |
| 277 | N277K | 0.81 |
| 277 | N277R | 0.77 |
| 278 | L278A | 0.84 |
| 278 | L278G | 0.81 |
| 278 | L278H | 0.66 |
| 278 | L278I | 0.73 |
| 278 | L278K | 0.72 |
| 278 | L278M | 0.69 |
| 278 | L278P | 0.83 |
| 278 | L278Q | 0.65 |
| 278 | L278R | 0.65 |
| 278 | L278S | 0.89 |
| 278 | L278V | 0.6 |
| 279 | Y279M | 0.85 |
| 279 | Y279T | 0.89 |
| 279 | Y279W | 0.49 |
| 280 | T280A | 0.8 |
| 280 | T280E | 0.61 |
| 280 | T280F | 0.71 |
| 280 | T280H | 0.86 |
| 280 | T280M | 0.89 |
| 280 | T280Q | 0.73 |
| 281 | V281A | 0.87 |
| 281 | V281I | 0.75 |
| 281 | V281L | 0.86 |
| 282 | R282E | 0.74 |
| 282 | R282F | 0.75 |
| 282 | R282H | 0.7 |
| 282 | R282I | 0.63 |
| 282 | R282K | 0.74 |
| 282 | R282N | 0.71 |
| 282 | R282S | 0.78 |
| 282 | R282T | 0.81 |
| 282 | R282V | 0.71 |
| 283 | T283R | 0.72 |
| 283 | T283V | 0.71 |
| 284 | E284A | 0.72 |
| 284 | E284D | 0.72 |
| 284 | E284F | 0.88 |
| 284 | E284H | 0.76 |
| 284 | E284L | 0.7 |
| 284 | E284M | 0.89 |
| 284 | E284N | 0.85 |
| 284 | E284Q | 0.86 |
| 284 | E284R | 0.79 |
| 284 | E284Y | 0.71 |
| 285 | V285I | 0.85 |
| 285 | V285T | 0.6 |
| 286 | L286A | 0.89 |
| 286 | L286C | 0.84 |
| 286 | L286D | 0.88 |
| 286 | L286F | 0.57 |
| 286 | L286N | 0.74 |
| 286 | L286R | 0.81 |
| 286 | L286T | 0.79 |
| 286 | L286Y | 0.81 |
| 287 | N287I | 0.83 |
| 287 | N287L | 0.83 |
| 288 | G288F | 0.84 |
| 288 | G288L | 0.83 |
| 288 | G288S | 0.79 |
| 291 | V291C | 0.84 |
| 291 | V291D | 0.59 |
| 291 | V291F | 0.78 |
| 291 | V291G | 0.84 |
| 291 | V291T | 0.75 |
| 291 | V291Y | 0.74 |
| 292 | L292H | 0.67 |
| 293 | D293E | 0.89 |
| 293 | D293F | 0.88 |
| 293 | D293S | 0.66 |
| 293 | D293V | 0.89 |
| 294 | T294G | 0.83 |
| 295 | Y295I | 0.87 |
| 295 | Y295R | 0.87 |
| 295 | Y295W | 0.63 |
| 296 | D296L | 0.86 |
| 296 | D296N | 0.88 |
| 296 | D296R | 0.66 |
| 296 | D296V | 0.75 |
| 297 | T297G | 0.8 |
| 297 | T297Q | 0.86 |
| 297 | T297R | 0.88 |
| 298 | E298G | 0.86 |
| 301 | F301L | 0.88 |
| 302 | R302E | 0.84 |
| 302 | R302N | 0.79 |
| 303 | W303S | 0.75 |
| 305 | G305F | 0.83 |
| 305 | G305W | 0.44 |
| 306 | F306D | 0.9 |
| 306 | F306K | 0.73 |
| 306 | F306L | 0.72 |
| 306 | F306S | 0.84 |
| 307 | D307G | 0.78 |
| 307 | D307L | 0.85 |
| 307 | D307M | 0.87 |
| 307 | D307N | 0.85 |
| 307 | D307Q | 0.83 |
| 308 | A308C | 0.88 |
| 308 | A308E | 0.78 |
| 308 | A308M | 0.64 |
| 308 | A308Y | 0.64 |
| 309 | T309M | 0.87 |
| 309 | T309S | 0.78 |
| 311 | G311E | 0.87 |
| 311 | G311Q | 0.8 |
| 311 | G311R | 0.81 |
| 311 | G311V | 0.69 |
| 312 | F312R | 0.72 |
| 314 | L314C | 0.69 |
| 314 | L314T | 0.71 |
| 316 | G316A | 0.82 |
| 316 | G316C | 0.82 |
| 316 | G316S | 0.73 |
| 317 | E317D | 0.82 |
| 317 | E317H | 0.9 |
| 317 | E317I | 0.87 |
| 317 | E317K | 0.72 |
| 318 | K318G | 0.83 |
| 318 | K318L | 0.82 |
| 318 | K318V | 0.9 |
| 320 | K320C | 0.61 |
| 320 | K320F | 0.75 |
| 320 | K320L | 0.76 |
| 320 | K320R | 0.74 |
| 320 | K320T | 0.79 |
| 321 | L321C | 0.81 |
| 321 | L321G | 0.88 |
| 321 | L321V | 0.89 |
| 322 | K322F | 0.61 |
| 322 | K322N | 0.85 |
| 322 | K322P | 0.71 |
| 322 | K322Q | 0.83 |
| 322 | K322R | 0.89 |
| 322 | K322S | 0.78 |
| 325 | S325D | 0.67 |
| 325 | S325T | 0.65 |
| 326 | M326E | 0.54 |
| 326 | M326G | 0.51 |
| 326 | M326S | 0.84 |
| 326 | M326Y | 0.57 |
| 329 | D329S | 0.74 |
| 329 | D329T | 0.87 |
| 337 | A337Q | 0.89 |
| 338 | N338V | 0.87 |
| 340 | R340C | 0.72 |
| 341 | A341S | 0.75 |
| 342 | I342K | 0.54 |
| 343 | E343H | 0.88 |
| 343 | E343S | 0.85 |
| 344 | R344L | 0.83 |
| 345 | Q345G | 0.53 |
| 345 | Q345K | 0.56 |
| 346 | V346C | 0.79 |
| 346 | V346F | 0.88 |
| 346 | V346L | 0.88 |
| 347 | E347C | 0.72 |
| 347 | E347F | 0.72 |
| 347 | E347R | 0.71 |
| 347 | E347S | 0.82 |
| 349 | L349Q | 0.78 |
| 351 | K351N | 0.89 |
| 351 | K351V | 0.73 |
| 352 | M352G | 0.71 |
| 352 | M352W | 0.7 |
| 355 | N355M | 0.77 |
| 355 | N355R | 0.83 |
| 355 | N355W | 0.78 |
| 362 | N362G | 0.89 |
| 365 | A365E | 0.74 |
| 366 | K366A | 0.79 |
| 366 | K366E | 0.85 |
| 367 | A367C | 0.9 |
| 368 | L368E | 0.53 |
| 369 | I369R | 0.78 |
| 370 | D370C | 0.84 |
| 370 | D370S | 0.76 |
| 371 | V371D | 0.69 |
| 371 | V371S | 0.79 |
| 374 | E374K | 0.81 |
| 374 | E374R | 0.71 |
| 378 | L378W | 0.81 |
| 380 | V380M | 0.87 |
| 381 | E381L | 0.9 |
| 383 | V383K | 0.77 |
| 383 | V383L | 0.75 |
| 386 | M386Q | 0.86 |
| 387 | W387L | 0.42 |
| 388 | N388A | 0.73 |
| 388 | N388R | 0.58 |
| 389 | R389N | 0.75 |
| 390 | S390G | 0.55 |
| 390 | S390N | 0.83 |
| 390 | S390Q | 0.18 |
| 390 | S390V | 0.7 |
| 391 | K391E | 0.36 |
| 392 | N392D | 0.02 |
| 393 | G393A | 0.67 |
| 393 | G393N | 0.77 |
| 393 | G393S | 0.63 |
| 394 | N394L | 0.11 |
| 395 | T395H | 0.31 |
| 395 | T395I | 0.8 |
| 395 | T395M | 0.74 |
| 395 | T395N | 0.15 |
| 395 | T395Q | 0.86 |
| 395 | T395S | 0.41 |
| 395 | T395W | 0.07 |
| 396 | E396M | 0.64 |
| 398 | Y398N | 0.33 |
| 400 | K400D | 0.86 |
| 400 | K400E | 0.89 |
| 400 | K400P | 0.43 |
| 400 | K400Q | 0.7 |
| 400 | K400S | 0.32 |
| 401 | W401H | 0.77 |
| 401 | W401L | 0.64 |
| 401 | W401M | 0.81 |
| 401 | W401R | 0.85 |
| 402 | F402T | 0.85 |
| 403 | G403P | 0.71 |
| 403 | G403S | 0.76 |
| 404 | Q404H | 0.42 |
| 404 | Q404R | 0.77 |
| 405 | A405H | 0.55 |
| 405 | A405P | 0.72 |
| 407 | A407M | 0.86 |
| 407 | A407S | 0.82 |
| 407 | A407T | 0.69 |
| 407 | A407W | 0.89 |
| 408 | G408D | 0.84 |
| 408 | G408I | 0.71 |
| 408 | G408M | 0.72 |
| 408 | G408N | 0.71 |
| 408 | G408W | 0.51 |
| 409 | D409N | 0.59 |
| 409 | D409W | 0.84 |
| 410 | N410C | 0.89 |
| 411 | A411N | 0.86 |
| 411 | A411V | 0.7 |
| 412 | V412M | 0.86 |
| 413 | L413D | 0.87 |
| 413 | L413E | 0.85 |
| 413 | L413F | 0.83 |
| 413 | L413I | 0.77 |
| 413 | L413P | 0.41 |
| 413 | L413T | 0.77 |
| 414 | G414A | 0.41 |
| 414 | G414C | 0.8 |
| 414 | G414R | 0.51 |
| 414 | G414W | 0.86 |
| 415 | G415R | 0.68 |
| 416 | D416I | 0.41 |
| 416 | D416R | 0.64 |
| 416 | D416T | 0.81 |
| 416 | D416Y | 0.58 |
| 417 | K417C | 0.77 |
| 417 | K417G | 0.77 |
| 417 | K417T | 0.79 |
| 418 | D418L | 0.89 |
| 418 | D418P | 0.59 |
| 418 | D418R | 0.26 |
| 418 | D418Y | 0.8 |
| 419 | E419M | 0.54 |
| 419 | E419R | 0.58 |
| 419 | E419W | 0.68 |
| 420 | T420E | 0.72 |
| 420 | T420F | 0.73 |
| 420 | T420G | 0.76 |
| 420 | T420K | 0.5 |
| 420 | T420R | 0.37 |
| 420 | T420V | 0.71 |
| 421 | W421L | 0.73 |
| 421 | W421Q | 0.63 |
| 421 | W421S | 0.82 |
| 422 | A422T | 0.62 |
| 422 | A422V | 0.83 |
| 423 | K423D | 0.43 |
| 423 | K423M | 0.86 |
| 424 | F424C | 0.58 |
| 424 | F424L | 0.44 |
| 424 | F424N | 0.69 |
| 424 | F424T | 0.87 |
| 425 | D425E | 0.87 |
| 426 | L426M | 0.52 |
| 426 | L426Q | 0.73 |
| 427 | T427G | 0.77 |
| 427 | T427K | 0.69 |
| 427 | T427M | 0.67 |
| 427 | T427P | 0.73 |
| 427 | T427R | 0.86 |
| 428 | S428F | 0.72 |
| 428 | S428K | 0.74 |
| 428 | S428W | 0.87 |
| 430 | I430C | 0.76 |
| 430 | I430D | 0.89 |
| 430 | I430E | 0.82 |
| 430 | I430Q | 0.78 |
| 430 | I430S | 0.7 |
| 430 | I430T | 0.78 |
| 430 | I430W | 0.88 |
| 431 | N431D | 0.79 |
| 431 | N431E | 0.78 |
| 431 | N431G | 0.86 |
| 431 | N431R | 0.83 |
| 431 | N431V | 0.89 |
| 431 | N431Y | 0.8 |
| 432 | R432A | 0.78 |
| 432 | R432E | 0.78 |
| 432 | R432N | 0.78 |
| 432 | R432V | 0.85 |
| 433 | D433H | 0.84 |
| 434 | R434L | 0.89 |
| 434 | R434M | 0.89 |
| 434 | R434P | 0.81 |
| 434 | R434S | 0.78 |
| 435 | N435K | 0.79 |
| 435 | N435M | 0.82 |
| 435 | N435R | 0.68 |
| 435 | N435W | 0.79 |
| 436 | A436D | 0.84 |
| 436 | A436E | 0.86 |
| 436 | A436I | 0.81 |
| 436 | A436Q | 0.85 |
| 436 | A436S | 0.85 |
| 437 | P437A | 0.76 |
| 437 | P437D | 0.85 |
| 437 | P437Q | 0.65 |
| 437 | P437R | 0.62 |
| 437 | P437S | 0.83 |
| 437 | P437V | 0.74 |
| 439 | V439E | 0.69 |
| 439 | V439G | 0.5 |
| 439 | V439I | 0.75 |
| 439 | V439Q | 0.82 |
| 439 | V439T | 0.56 |
| 439 | V439Y | 0.73 |
| 440 | I440C | 0.8 |
| 440 | I440D | 0.81 |
| 440 | I440F | 0.72 |
| 440 | I440K | 0.7 |
| 440 | I440P | 0.86 |
| 440 | I440R | 0.63 |
| 440 | I440W | 0.78 |
| 441 | M441A | 0.7 |
| 441 | M441E | 0.76 |
| 441 | M441G | 0.83 |
| 441 | M441Q | 0.78 |
| 441 | M441V | 0.8 |
| 442 | W442M | 0.88 |
| 442 | W442P | 0.79 |
| 442 | W442Q | 0.89 |
| 442 | W442R | 0.55 |
| 443 | S443D | 0.86 |
| 443 | S443Y | 0.56 |
| 444 | L444C | 0.9 |
| 444 | L444D | 0.77 |
| 444 | L444G | 0.7 |
| 444 | L444H | 0.88 |
| 444 | L444K | 0.7 |
| 444 | L444Q | 0.76 |
| 444 | L444W | 0.73 |
| 445 | G445A | 0.58 |
| 445 | G445C | 0.41 |
| 445 | G445V | 0.39 |
| 446 | N446T | 0.67 |
| 448 | M448A | 0.74 |
| 448 | M448D | 0.72 |
| 448 | M448I | 0.71 |
| 448 | M448P | 0.78 |
| 448 | M448Q | 0.68 |
| 448 | M448S | 0.51 |
| 448 | M448V | 0.68 |
| 448 | M448W | 0.84 |
| 452 | I452G | 0.67 |
| 452 | I452M | 0.63 |
| 453 | S453F | 0.7 |
| 455 | S455A | 0.8 |
| 455 | S455K | 0.8 |
| 455 | S455W | 0.63 |
| 456 | V456A | 0.8 |
| 456 | V456L | 0.8 |
| 456 | V456W | 0.83 |
| 457 | S457E | 0.89 |
| 457 | S457K | 0.79 |
| 457 | S457L | 0.89 |
| 457 | S457M | 0.87 |
| 457 | S457P | 0.71 |
| 458 | G458C | 0.89 |
| 458 | G458F | 0.9 |
| 458 | G458L | 0.81 |
| 458 | G458Q | 0.82 |
| 458 | G458V | 0.7 |
| 458 | G458W | 0.65 |
| 459 | F459A | 0.88 |
| 459 | F459E | 0.81 |
| 459 | F459G | 0.88 |
| 459 | F459N | 0.66 |
| 459 | F459S | 0.58 |
| 459 | F459T | 0.88 |
| 459 | F459W | 0.81 |
| 460 | P460C | 0.86 |
| 460 | P460Q | 0.82 |
| 460 | P460W | 0.81 |
| 461 | A461D | 0.79 |
| 461 | A461N | 0.74 |
| 461 | A461Q | 0.85 |
| 461 | A461V | 0.85 |
| 461 | A461Y | 0.73 |
| 462 | T462C | 0.77 |
| 462 | T462F | 0.76 |
| 462 | T462M | 0.83 |
| 462 | T462S | 0.8 |
| 463 | S463G | 0.85 |
| 463 | S463K | 0.73 |
| 463 | S463Q | 0.73 |
| 463 | S463R | 0.87 |
| 463 | S463T | 0.77 |
| 463 | S463V | 0.69 |
| 464 | A464E | 0.85 |
| 464 | A464H | 0.84 |
| 464 | A464L | 0.83 |
| 464 | A464M | 0.76 |
| 464 | A464P | 0.67 |
| 464 | A464V | 0.67 |
| 465 | K465F | 0.67 |
| 465 | K465R | 0.82 |
| 465 | K465W | 0.86 |
| 466 | L466A | 0.63 |
| 466 | L466C | 0.83 |
| 466 | L466D | 0.85 |
| 466 | L466E | 0.62 |
| 466 | L466F | 0.72 |
| 466 | L466M | 0.71 |
| 466 | L466Q | 0.77 |
| 466 | L466S | 0.68 |
| 466 | L466V | 0.59 |
| 466 | L466Y | 0.8 |
| 467 | V467A | 0.89 |
| 467 | V467C | 0.89 |
| 467 | V467D | 0.77 |
| 467 | V467E | 0.8 |
| 467 | V467G | 0.73 |
| 467 | V467W | 0.75 |
| 468 | A468E | 0.77 |
| 468 | A468F | 0.8 |
| 468 | A468K | 0.78 |
| 468 | A468L | 0.8 |
| 468 | A468P | 0.85 |
| 468 | A468S | 0.85 |
| 468 | A468V | 0.71 |
| 468 | A468W | 0.68 |
| 469 | W469A | 0.8 |
| 469 | W469C | 0.84 |
| 469 | W469D | 0.88 |
| 469 | W469G | 0.73 |
| 469 | W469M | 0.78 |
| 469 | W469V | 0.87 |
| 469 | W469Y | 0.85 |
| 470 | T470E | 0.88 |
| 470 | T470L | 0.83 |
| 470 | T470M | 0.68 |
| 470 | T470Q | 0.88 |
| 471 | K471G | 0.87 |
| 471 | K471W | 0.75 |
| 473 | A473E | 0.86 |
| 474 | D474A | 0.88 |
| 474 | D474K | 0.83 |
| 474 | D474R | 0.64 |
| 475 | S475F | 0.76 |
| 475 | S475Q | 0.83 |
| 475 | S475V | 0.83 |
| 476 | T476C | 0.83 |
| 477 | R477C | 0.89 |
| 478 | P478G | 0.76 |
| 478 | P478L | 0.74 |
| 479 | M479I | 0.65 |
| 479 | M479R | 0.49 |
| 479 | M479W | 0.77 |
| 480 | T480C | 0.61 |
| 480 | T480G | 0.69 |
| 487 | K487G | 0.8 |
| 487 | K487N | 0.59 |
| 488 | A488G | 0.77 |
| 488 | A488H | 0.62 |
| 488 | A488N | 0.34 |
| 488 | A488S | 0.68 |
| 491 | N491A | 0.73 |
| 491 | N491E | 0.82 |
| 491 | N491W | 0.09 |
| 492 | E492A | 0.64 |
| 492 | E492W | 0.76 |
| 493 | S493M | 0.47 |
| 493 | S493V | 0.9 |
| 495 | T495R | 0.68 |
| 495 | T495V | 0.78 |
| 495 | T495W | 0.52 |
| 496 | M496A | 0.8 |
| 496 | M496F | 0.72 |
| 496 | M496T | 0.85 |
| 498 | D498A | 0.76 |
| 498 | D498C | 0.5 |
| 498 | D498M | 0.71 |
| 498 | D498S | 0.7 |
| 499 | N499K | 0.89 |
| 499 | N499R | 0.54 |
| 499 | N499Y | 0.2 |
| 500 | L500A | 0.49 |
| 500 | L500E | 0.9 |
| 500 | L500N | 0.74 |
| 500 | L500V | 0.87 |
| 501 | T501C | 0.87 |
| 501 | T501G | 0.72 |
| 501 | T501M | 0.7 |
| 502 | A502L | 0.74 |
| 502 | A502Q | 0.81 |
| 503 | N503A | 0.8 |
| 503 | N503E | 0.74 |
| 503 | N503M | 0.73 |
| 503 | N503S | 0.9 |
| 504 | G504H | 0.29 |
| 504 | G504K | 0.74 |
| 504 | G504P | 0.69 |
| 505 | G505A | 0.74 |
| 505 | G505E | 0.73 |
| 505 | G505N | 0.67 |
| 506 | V506D | 0.65 |
| 506 | V506G | 0.66 |
| 506 | V506I | 0.85 |
| 506 | V506L | 0.76 |
| 506 | V506P | 0.79 |
| 506 | V506S | 0.77 |
| 506 | V506T | 0.81 |
| 506 | V506W | 0.86 |
| 507 | V507A | 0.75 |
| 507 | V507G | 0.74 |
| 507 | V507L | 0.38 |
| 507 | V507N | 0.72 |
| 507 | V507S | 0.75 |
| 507 | V507T | 0.82 |
| 510 | N510Q | 0.8 |
| 511 | Y511K | 0.82 |
| 512 | S512G | 0.75 |
| 513 | D513C | 0.86 |
| 513 | D513K | 0.6 |
| 513 | D513L | 0.45 |
| 513 | D513M | 0.9 |
| 513 | D513Q | 0.82 |
| 513 | D513W | 0.89 |
| 514 | G514F | 0.58 |
| 514 | G514L | 0.77 |
| 514 | G514N | 0.84 |
| 514 | G514P | 0.81 |
| 514 | G514R | 0.84 |
| 515 | A515C | 0.66 |
| 515 | A515D | 0.78 |
| 515 | A515K | 0.62 |
| 515 | A515R | 0.66 |
| 517 | Y517N | 0.74 |
| 518 | D518Q | 0.75 |
| 521 | R521N | 0.35 |
| 522 | T522G | 0.61 |
| 522 | T522I | 0.84 |
| 522 | T522N | 0.29 |
| 525 | P525D | 0.87 |
| 525 | P525G | 0.8 |
| 525 | P525T | 0.53 |
| 525 | P525V | 0.89 |
| 526 | S526I | 0.9 |
| 526 | S526W | 0.88 |
| 527 | W527V | 0.84 |
| 528 | A528C | 0.72 |
| 528 | A528I | 0.79 |
| 529 | I529F | 0.87 |
| 529 | I529L | 0.85 |
| 529 | I529Y | 0.81 |
| 530 | Y530A | 0.74 |
| 530 | Y530M | 0.62 |
| 531 | G531E | 0.63 |
| 531 | G531S | 0.67 |
| 531 | G531T | 0.59 |
| 534 | T534A | 0.69 |
| 534 | T534I | 0.7 |
| 534 | T534Q | 0.9 |
| 535 | A535M | 0.65 |
| 537 | A537D | 0.68 |
| 539 | N539G | 0.9 |
| 539 | N539W | 0.77 |
| 540 | S540G | 0.64 |
| 540 | S540M | 0.8 |
| 542 | G542E | 0.85 |
| 542 | G542Q | 0.65 |
| 542 | G542S | 0.89 |
| 542 | G542T | 0.82 |
| 543 | I543V | 0.68 |
| 543 | I543W | 0.82 |
| 545 | N545I | 0.88 |
| 545 | N545Q | 0.74 |
| 545 | N545S | 0.69 |
| 546 | R546A | 0.81 |
| 546 | R546C | 0.72 |
| 546 | R546L | 0.46 |
| 546 | R546P | 0.53 |
| 546 | R546S | 0.14 |
| 547 | T547A | 0.53 |
| 547 | T547D | 0.68 |
| 547 | T547H | 0.85 |
| 547 | T547N | 0.7 |
| 547 | T547S | 0.56 |
| 548 | T548D | 0.89 |
| 548 | T548E | 0.7 |
| 548 | T548F | 0.51 |
| 548 | T548K | 0.5 |
| 548 | T548L | 0.73 |
| 548 | T548P | 0.41 |
| 548 | T548W | 0.8 |
| 549 | G549F | 0.78 |
| 550 | G550S | 0.28 |
| 551 | A551D | 0.89 |
| 551 | A551I | 0.64 |
| 551 | A551Q | 0.47 |
| 553 | S553C | 0.85 |
| 553 | S553F | 0.75 |
| 553 | S553N | 0.53 |
| 553 | S553T | 0.85 |
| 553 | S553V | 0.86 |
| 554 | S554F | 0.89 |
| 554 | S554N | 0.33 |
| 554 | S554R | 0.83 |
| 554 | S554T | 0.82 |
| 554 | S554V | 0.7 |
| 554 | S554W | 0.8 |
| 555 | D555S | 0.23 |
| 556 | K556A | 0.8 |
| 556 | K556W | 0.37 |
| 557 | Q557F | 0.9 |
| 557 | Q557R | 0.73 |
| 557 | Q557S | 0.83 |
| 558 | L558I | 0.89 |
| 559 | T559A | 0.81 |
| 559 | T559I | 0.88 |
| 561 | Y561R | 0.66 |
| 563 | N563R | 0.85 |
| 563 | N563S | 0.67 |
| 570 | A570K | 0.34 |
| 570 | A570M | 0.59 |
| 570 | A570S | 0.86 |
| 570 | A570T | 0.88 |
| 570 | A570W | 0.62 |
| 571 | V571W | 0.89 |
| 572 | A572S | 0.56 |
| 573 | S573G | 0.78 |
| 573 | S573K | 0.63 |
| 573 | S573Y | 0.81 |
| 574 | S574K | 0.61 |
| 574 | S574Q | 0.66 |
| 574 | S574W | 0.45 |
| 575 | A575M | 0.83 |
| 575 | A575V | 0.63 |
| 576 | W576A | 0.82 |
| 576 | W576F | 0.77 |
| 576 | W576V | 0.78 |
| 576 | W576Y | 0.54 |
| 577 | Y577G | 0.68 |
| 577 | Y577I | 0.86 |
| 577 | Y577L | 0.52 |
| 578 | D578E | 0.89 |
| 578 | D578M | 0.79 |
| 578 | D578N | 0.64 |
| 579 | V579E | 0.71 |
| 579 | V579G | 0.52 |
| 579 | V579L | 0.78 |
| 579 | V579T | 0.62 |
| 580 | V580A | 0.77 |
| 580 | V580E | 0.89 |
| 580 | V580K | 0.67 |
| 580 | V580L | 0.77 |
| 580 | V580S | 0.7 |
| 581 | Q581F | 0.75 |
| 581 | Q581G | 0.7 |
| 581 | Q581P | 0.77 |
| 581 | Q581R | 0.8 |
| 581 | Q581S | 0.73 |
| 582 | R582D | 0.89 |
| 582 | R582Y | 0.71 |
| 584 | F584E | 0.67 |
| 585 | V585I | 0.57 |
| 585 | V585M | 0.78 |
| 585 | V585Q | 0.81 |
| 586 | A586C | 0.82 |
| 586 | A586H | 0.79 |
| 586 | A586K | 0.48 |
| 587 | G587A | 0.71 |
| 587 | G587C | 0.62 |
| 588 | T588C | 0.71 |
| 588 | T588D | 0.52 |
| 588 | T588G | 0.87 |
| 588 | T588I | 0.61 |
| 588 | T588L | 0.76 |
| 588 | T588M | 0.77 |
| 589 | Y589A | 0.82 |
| 589 | Y589V | 0.69 |
| 589 | Y589W | 0.6 |
| 590 | V590A | 0.75 |
| 590 | V590H | 0.66 |
| 590 | V590I | 0.87 |
| 592 | T592C | 0.5 |
| 592 | T592Q | 0.81 |
| 592 | T592S | 0.32 |
| 593 | G593I | 0.79 |
| 594 | F594I | 0.81 |
| 594 | F594L | 0.34 |
| 594 | F594M | 0.81 |
| 595 | D595E | 0.68 |
| 595 | D595Q | 0.49 |
| 595 | D595S | 0.68 |
| 597 | L597C | 0.89 |
| 597 | L597E | 0.78 |
| 606 | T606S | 0.68 |
| 607 | G607A | 0.88 |
| 608 | S608M | 0.84 |
| 608 | S608V | 0.11 |
| 609 | G609H | 0.84 |
| 609 | G609L | 0.78 |
| 609 | G609N | 0.44 |
| 609 | G609R | 0.25 |
| 610 | A610D | 0.83 |
| 610 | A610F | 0.75 |
| 610 | A610M | 0.57 |
| 611 | V611K | 0.54 |
| 612 | G612N | 0.72 |
| 612 | G612T | 0.76 |
| 614 | W614F | 0.9 |
| 614 | W614P | 0.69 |
| 615 | P615L | 0.75 |
| 615 | P615S | 0.87 |
| 616 | S616A | 0.84 |
| 616 | S616W | 0.75 |
| 619 | N619A | 0.79 |
| 620 | S620G | 0.59 |
| 624 | I624A | 0.76 |
| 624 | I624V | 0.87 |
| 627 | T627F | 0.81 |
| 627 | T627G | 0.84 |
| 630 | F630A | 0.86 |
| 630 | F630D | 0.64 |
| 630 | F630G | 0.32 |
| 630 | F630S | 0.81 |
| 631 | P631A | 0.68 |
| 631 | P631G | 0.82 |
| 631 | P631H | 0.86 |
| 631 | P631S | 0.88 |
| 631 | P631V | 0.79 |
| 632 | K632T | 0.75 |
| 634 | T634A | 0.8 |
| 634 | T634E | 0.71 |
| 635 | Y635R | 0.57 |
| 638 | Y638A | 0.78 |
| 638 | Y638W | 0.71 |
| 639 | Q639D | 0.86 |
| 639 | Q639N | 0.9 |
| 640 | S640C | 0.89 |
| 640 | S640D | 0.9 |
| 641 | Q641T | 0.85 |
| 642 | W642I | 0.85 |
| 644 | D644C | 0.33 |
| 644 | D644G | 0.86 |
| 650 | H650E | 0.62 |
| 650 | H650R | 0.19 |
| 651 | I651F | 0.82 |
| 654 | A654K | 0.73 |
| 654 | A654M | 0.78 |
| 656 | N656A | 0.9 |
| 656 | N656V | 0.46 |
| 659 | V659D | 0.83 |
| 659 | V659N | 0.71 |
| 669 | P669F | 0.88 |
| 670 | V670C | 0.1 |
| 673 | Y673E | 0.75 |
| 673 | Y673G | 0.79 |
| 673 | Y673I | 0.8 |
| 673 | Y673R | 0.67 |
| 673 | Y673W | 0.89 |
| 674 | T674C | 0.8 |
| 674 | T674G | 0.85 |
| 674 | T674H | 0.83 |
| 675 | D675E | 0.83 |
| 675 | D675P | 0.66 |
| 675 | D675Q | 0.73 |
| 675 | D675S | 0.8 |
| 676 | A676C | 0.71 |
| 676 | A676E | 0.71 |
| 676 | A676G | 0.86 |
| 676 | A676K | 0.89 |
| 676 | A676S | 0.83 |
| 676 | A676W | 0.83 |
| 677 | A677E | 0.74 |
| 677 | A677G | 0.67 |
| 677 | A677L | 0.86 |
| 677 | A677R | 0.87 |
| 677 | A677V | 0.81 |
| 677 | A677Y | 0.85 |
| 678 | K678A | 0.71 |
| 678 | K678C | 0.87 |
| 678 | K678T | 0.67 |
| 678 | K678V | 0.89 |
| 679 | V679G | 0.64 |
| 679 | V679T | 0.87 |
| 679 | V679Y | 0.72 |
| 680 | K680A | 0.89 |
| 680 | K680E | 0.8 |
| 680 | K680G | 0.89 |
| 680 | K680H | 0.78 |
| 680 | K680L | 0.84 |
| 680 | K680Q | 0.88 |
| 680 | K680S | 0.86 |
| 680 | K680V | 0.74 |
| 680 | K680W | 0.84 |
| 681 | L681E | 0.72 |
| 681 | L681F | 0.64 |
| 681 | L681G | 0.83 |
| 681 | L681M | 0.9 |
| 681 | L681S | 0.75 |
| 681 | L681T | 0.8 |
| 682 | Y682D | 0.87 |
| 682 | Y682E | 0.74 |
| 682 | Y682I | 0.89 |
| 682 | Y682M | 0.88 |
| 682 | Y682S | 0.84 |
| 682 | Y682W | 0.84 |
| 683 | F683H | 0.83 |
| 683 | F683L | 0.9 |
| 683 | F683M | 0.73 |
| 683 | F683Q | 0.7 |
| 683 | F683R | 0.85 |
| 684 | T684A | 0.87 |
| 684 | T684D | 0.89 |
| 684 | T684G | 0.69 |
| 684 | T684L | 0.87 |
| 684 | T684S | 0.83 |
| 684 | T684V | 0.54 |
| 685 | P685A | 0.8 |
| 685 | P685E | 0.82 |
| 685 | P685I | 0.8 |
| 685 | P685L | 0.85 |
| 685 | P685W | 0.84 |
| 686 | K686A | 0.89 |
| 686 | K686I | 0.8 |
| 686 | K686M | 0.79 |
| 686 | K686T | 0.85 |
| 686 | K686V | 0.84 |
| 687 | G687A | 0.78 |
| 687 | G687K | 0.82 |
| 687 | G687N | 0.88 |
| 687 | G687Q | 0.79 |
| 687 | G687R | 0.74 |
| 688 | S688C | 0.9 |
| 688 | S688D | 0.85 |
| 688 | S688E | 0.74 |
| 688 | S688G | 0.81 |
| 688 | S688P | 0.88 |
| 688 | S688T | 0.79 |
| 689 | T689A | 0.86 |
| 689 | T689D | 0.78 |
| 689 | T689E | 0.85 |
| 689 | T689L | 0.84 |
| 689 | T689P | 0.74 |
| 689 | T689S | 0.87 |
| 689 | T689Y | 0.88 |
| 690 | E690M | 0.87 |
| 690 | E690P | 0.89 |
| 690 | E690T | 0.88 |
| 690 | E690V | 0.73 |
| 691 | K691N | 0.83 |
| 691 | K691S | 0.84 |
| 693 | L693A | 0.88 |
| 696 | E696A | 0.77 |
| 696 | E696L | 0.86 |
| 697 | K697E | 0.8 |
| 698 | S698C | 0.89 |
| 698 | S698I | 0.77 |
| 698 | S698L | 0.7 |
| 698 | S698P | 0.87 |
| 698 | S698T | 0.73 |
| 700 | T700C | 0.33 |
| 700 | T700E | 0.81 |
| 700 | T700G | 0.84 |
| 700 | T700K | 0.85 |
| 701 | K701E | 0.89 |
| 701 | K701W | 0.89 |
| 703 | T703E | 0.89 |
| 703 | T703I | 0.8 |
| 703 | T703W | 0.82 |
| 704 | T704M | 0.7 |
| 704 | T704R | 0.9 |
| 704 | T704Y | 0.66 |
| 705 | A705C | 0.77 |
| 705 | A705E | 0.61 |
| 705 | A705N | 0.75 |
| 705 | A705V | 0.89 |
| 706 | A706G | 0.89 |
| 706 | A706Y | 0.72 |
| 708 | Y708C | 0.78 |
| 708 | Y708G | 0.82 |
| 708 | Y708K | 0.86 |
| 708 | Y708P | 0.88 |
| 708 | Y708T | 0.8 |
| 709 | T709M | 0.5 |
| 710 | Y710C | 0.84 |
| 710 | Y710D | 0.66 |
| 710 | Y710G | 0.8 |
| 710 | Y710M | 0.82 |
| 710 | Y710N | 0.71 |
| 710 | Y710W | 0.68 |
| 711 | Q711G | 0.87 |
| 711 | Q711L | 0.73 |
| 711 | Q711M | 0.78 |
| 711 | Q711Y | 0.68 |
| 712 | V712G | 0.86 |
| 712 | V712R | 0.87 |
| 713 | Y713A | 0.81 |
| 713 | Y713L | 0.83 |
| 713 | Y713Q | 0.88 |
| 714 | E714D | 0.83 |
| 714 | E714M | 0.9 |
| 714 | E714R | 0.81 |
| 714 | E714S | 0.8 |
| 714 | E714V | 0.79 |
| 714 | E714Y | 0.85 |
| 716 | A716C | 0.8 |
| 716 | A716G | 0.89 |
| 716 | A716L | 0.79 |
| 716 | A716V | 0.8 |
| 717 | D717C | 0.76 |
| 718 | K718L | 0.79 |
| 719 | D719L | 0.87 |
| 719 | D719P | 0.89 |
| 724 | K724R | 0.86 |
| 725 | N725D | 0.88 |
| 726 | M726A | 0.82 |
| 726 | M726E | 0.84 |
| 726 | M726K | 0.83 |
| 726 | M726V | 0.75 |
| 726 | M726W | 0.81 |
| 727 | Y727T | 0.85 |
| 728 | L728Q | 0.89 |
| 728 | L728T | 0.86 |
| 730 | W730A | 0.89 |
| 731 | N731A | 0.88 |
| 731 | N731S | 0.3 |
| 731 | N731Y | 0.86 |
| 732 | V732G | 0.82 |
| 732 | V732P | 0.83 |
| 732 | V732W | 0.78 |
| 733 | P733A | 0.83 |
| 733 | P733R | 0.73 |
| 734 | W734D | 0.84 |
| 734 | W734V | 0.28 |
| 735 | A735W | 0.8 |
| 736 | E736S | 0.88 |
| 738 | T738G | 0.89 |
| 739 | I739M | 0.89 |
| 739 | I739W | 0.87 |
| 739 | I739Y | 0.87 |
| 740 | S740E | 0.88 |
| 740 | S740H | 0.9 |
| 740 | S740L | 0.89 |
| 742 | E742D | 0.84 |
| 742 | E742M | 0.89 |
| 742 | E742Q | 0.8 |
| 743 | A743C | 0.86 |
| 743 | A743H | 0.85 |
| 743 | A743L | 0.86 |
| 744 | Y744A | 0.87 |
| 744 | Y744E | 0.84 |
| 744 | Y744I | 0.88 |
| 744 | Y744K | 0.9 |
| 745 | D745F | 0.16 |
| 746 | E746A | 0.82 |
| 747 | N747G | 0.86 |
| 747 | N747R | 0.65 |
| 748 | N748S | 0.88 |
| 749 | R749H | 0.9 |
| 749 | R749S | 0.84 |
| 749 | R749T | 0.88 |
| 750 | L750G | 0.85 |
| 751 | I751Q | 0.9 |
| 751 | I751S | 0.88 |
| 752 | P752A | 0.51 |
| 752 | P752H | 0.89 |
| 752 | P752L | 0.89 |
| 752 | P752S | 0.81 |
| 752 | P752V | 0.82 |
| 753 | E753Q | 0.89 |
| 754 | G754P | 0.45 |
| 755 | S755C | 0.84 |
| 755 | S755I | 0.83 |
| 756 | T756E | 0.83 |
| 756 | T756P | 0.86 |
| 756 | T756S | 0.55 |
| 756 | T756W | 0.86 |
| 758 | G758A | 0.84 |
| 759 | N759D | 0.87 |
| 759 | N759R | 0.88 |
| 760 | A760N | 0.53 |
| 760 | A760P | 0.87 |
| 760 | A760Q | 0.84 |
| 761 | S761K | 0.84 |
| 761 | S761Q | 0.9 |
| 762 | V762D | 0.84 |
| 762 | V762G | 0.84 |
| 762 | V762K | 0.9 |
| 765 | T765A | 0.82 |
| 765 | T765R | 0.89 |
| 765 | T765W | 0.86 |
| 766 | G766S | 0.89 |
| 768 | A768H | 0.87 |
| 771 | L771I | 0.71 |
| 772 | K772V | 0.62 |
| 773 | A773G | 0.82 |
| 773 | A773H | 0.51 |
| 773 | A773S | 0.77 |
| 773 | A773V | 0.51 |
| 774 | D774R | 0.66 |
| 775 | A775L | 0.36 |
| 775 | A775W | 0.26 |
| 776 | D776I | 0.18 |
| 776 | D776L | 0.26 |
| 776 | D776S | 0.46 |
| 777 | R777D | 0.31 |
| 777 | R777E | 0.52 |
| 777 | R777G | 0.36 |
| 777 | R777P | 0.31 |
| 778 | K778A | 0.73 |
| 778 | K778C | 0.83 |
| 778 | K778L | 0.68 |
| 779 | T779C | 0.86 |
| 780 | I780V | 0.7 |
| 781 | T781C | 0.79 |
| 781 | T781M | 0.68 |
| 781 | T781R | 0.73 |
| 782 | A782E | 0.74 |
| 782 | A782K | 0.72 |
| 782 | A782P | 0.64 |
| 783 | D783C | 0.26 |
| 783 | D783E | 0.13 |
| 783 | D783R | 0.21 |
| 784 | G784A | 0.77 |
| 784 | G784F | 0.84 |
| 784 | G784L | 0.02 |
| 784 | G784S | 0.55 |
| 784 | G784T | 0.61 |
| 786 | D786S | 0.88 |
| 787 | L787P | 0.38 |
| 787 | L787T | 0.57 |
| 787 | L787Y | 0.85 |
| 788 | S788A | 0.72 |
| 788 | S788G | 0.62 |
| 788 | S788I | 0.62 |
| 789 | Y789C | 0.85 |
| 789 | Y789D | 0.86 |
| 789 | Y789I | 0.82 |
| 790 | I790A | 0.71 |
| 790 | I790C | 0.68 |
| 790 | I790F | 0.78 |
| 791 | E791A | 0.9 |
| 791 | E791M | 0.89 |
| 791 | E791T | 0.75 |
| 792 | V792C | 0.88 |
| 792 | V792G | 0.36 |
| 792 | V792L | 0.84 |
| 793 | D793F | 0.89 |
| 793 | D793H | 0.48 |
| 793 | D793K | 0.79 |
| 793 | D793N | 0.83 |
| 794 | V794C | 0.63 |
| 794 | V794L | 0.89 |
| 794 | V794Q | 0.8 |
| 794 | V794T | 0.33 |
| 794 | V794W | 0.65 |
| 795 | T795P | 0.44 |
| 795 | T795Y | 0.85 |
| 796 | D796M | 0.83 |
| 796 | D796Q | 0.7 |
| 797 | A797K | 0.87 |
| 798 | N798G | 0.83 |
| 798 | N798P | 0.87 |
| 799 | G799M | 0.42 |
| 799 | G799Q | 0.59 |
| 800 | H800A | 0.53 |
| 800 | H800L | 0.55 |
| 800 | H800S | 0.89 |
| 801 | I801C | 0.76 |
| 801 | I801E | 0.86 |
| 802 | V802P | 0.81 |
| 803 | P803A | 0.86 |
| 803 | P803F | 0.74 |
| 803 | P803K | 0.79 |
| 804 | D804G | 0.66 |
| 804 | D804K | 0.72 |
| 804 | D804N | 0.83 |
| 804 | D804S | 0.74 |
| 805 | A805G | 0.89 |
| 807 | N807Q | 0.71 |
| 807 | N807V | 0.85 |
| 807 | N807W | 0.67 |
| 808 | R808C | 0.54 |
| 808 | R808N | 0.69 |
| 808 | R808Q | 0.75 |
| 809 | V809A | 0.83 |
| 809 | V809C | 0.74 |
| 811 | F811Y | 0.79 |
| 812 | D812E | 0.84 |
| 812 | D812F | 0.88 |
| 812 | D812I | 0.88 |
| 812 | D812Q | 0.69 |
| 813 | V813F | 0.88 |
| 813 | V813T | 0.57 |
| 814 | K814H | 0.11 |
| 814 | K814I | 0.82 |
| 814 | K814L | 0.69 |
| 815 | G815A | 0.48 |
| 815 | G815V | 0.66 |
| 816 | A816C | 0.41 |
| 816 | A816F | 0.47 |
| 816 | A816I | 0.89 |
| 816 | A816V | 0.79 |
| 817 | G817C | 0.89 |
| 817 | G817I | 0.8 |
| 817 | G817N | 0.8 |
| 817 | G817S | 0.84 |
| 818 | K818V | 0.87 |
| 819 | L819W | 0.82 |
| 820 | V820F | 0.87 |
| 820 | V820I | 0.87 |
| 820 | V820K | 0.69 |
| 821 | G821A | 0.8 |
| 821 | G821C | 0.88 |
| 821 | G821E | 0.52 |
| 821 | G821F | 0.68 |
| 821 | G821K | 0.87 |
| 821 | G821M | 0.87 |
| 821 | G821N | 0.67 |
| 821 | G821V | 0.78 |
| 821 | G821Y | 0.87 |
| 824 | N824C | 0.88 |
| 824 | N824G | 0.83 |
| 824 | N824Q | 0.85 |
| 826 | S826F | 0.74 |
| 826 | S826L | 0.36 |
| 826 | S826R | 0.77 |
| 827 | S827C | 0.81 |
| 827 | S827Q | 0.78 |
| 828 | P828C | 0.69 |
| 828 | P828I | 0.85 |
| 828 | P828L | 0.87 |
| 828 | P828Y | 0.77 |
| 829 | D829T | 0.9 |
| 829 | D829V | 0.9 |
| 830 | H830E | 0.63 |
| 830 | H830G | 0.71 |
| 830 | H830M | 0.76 |
| 830 | H830P | 0.78 |
| 830 | H830R | 0.35 |
| 830 | H830V | 0.69 |
| 831 | D831F | 0.64 |
| 831 | D831G | 0.82 |
| 831 | D831I | 0.59 |
| 831 | D831R | 0.7 |
| 831 | D831V | 0.6 |
| 832 | S832E | 0.87 |
| 832 | S832F | 0.84 |
| 832 | S832G | 0.76 |
| 832 | S832L | 0.73 |
| 832 | S832M | 0.79 |
| 832 | S832V | 0.89 |
| 833 | Y833I | 0.74 |
| 833 | Y833K | 0.69 |
| 833 | Y833P | 0.76 |
| 833 | Y833V | 0.69 |
| 834 | Q834F | 0.77 |
| 834 | Q834G | 0.82 |
| 835 | A835E | 0.63 |
| 835 | A835F | 0.9 |
| 835 | A835W | 0.57 |
| 836 | D836H | 0.44 |
| 836 | D836W | 0.74 |
| 837 | N837D | 0.61 |
| 837 | N837F | 0.9 |
| 837 | N837G | 0.71 |
| 837 | N837H | 0.78 |
| 837 | N837L | 0.74 |
| 837 | N837P | 0.87 |
| 837 | N837T | 0.84 |
| 838 | R838D | 0.88 |
| 838 | R838F | 0.86 |
| 838 | R838G | 0.57 |
| 838 | R838K | 0.87 |
| 838 | R838M | 0.61 |
| 838 | R838W | 0.77 |
| 839 | K839C | 0.9 |
| 839 | K839E | 0.71 |
| 839 | K839G | 0.88 |
| 839 | K839P | 0.88 |
| 839 | K839R | 0.83 |
| 841 | F841C | 0.69 |
| 841 | F841D | 0.88 |
| 841 | F841W | 0.66 |
| 842 | S842L | 0.86 |
| 842 | S842M | 0.75 |
| 843 | G843A | 0.85 |
| 843 | G843C | 0.77 |
| 844 | K844G | 0.18 |
| 844 | K844L | 0.89 |
| 844 | K844W | 0.82 |
| 845 | V845N | 0.79 |
| 845 | V845W | 0.68 |
| 846 | L846G | 0.83 |
| 846 | L846I | 0.79 |
| 846 | L846M | 0.72 |
| 846 | L846S | 0.89 |
| 847 | A847L | 0.87 |
| 849 | V849A | 0.78 |
| 849 | V849L | 0.65 |
| 849 | V849S | 0.65 |
| 849 | V849T | 0.84 |
| 850 | Q850C | 0.57 |
| 850 | Q850G | 0.47 |
| 850 | Q850I | 0.9 |
| 850 | Q850T | 0.87 |
| 850 | Q850V | 0.89 |
| 850 | Q850Y | 0.62 |
| 851 | S851L | 0.86 |
| 852 | T852D | 0.88 |
| 852 | T852G | 0.88 |
| 853 | K853N | 0.75 |
| 853 | K853Q | 0.75 |
| 853 | K853V | 0.81 |
| 854 | E854M | 0.83 |
| 854 | E854R | 0.81 |
| 857 | E857P | 0.68 |
| 857 | E857V | 0.84 |
| 858 | I858E | 0.75 |
| 858 | I858F | 0.85 |
| 858 | I858M | 0.77 |
| 858 | I858Q | 0.54 |
| 858 | I858Y | 0.53 |
| 859 | T859V | 0.7 |
| 860 | V860T | 0.68 |
| 862 | A862P | 0.84 |
| 862 | A862V | 0.69 |
| 863 | K863F | 0.6 |
| 863 | K863I | 0.72 |
| 863 | K863L | 0.86 |
| 863 | K863N | 0.54 |
| 863 | K863W | 0.65 |
| 864 | A864E | 0.86 |
| 864 | A864H | 0.89 |
| 864 | A864L | 0.74 |
| 870 | S870R | 0.5 |
| 871 | T871P | 0.72 |
| 877 | T877A | 0.67 |
| 879 | V879P | 0.83 |
| 880 | P880S | 0.73 |
| 881 | G881W | 0.64 |
| 884 | T884A | 0.67 |
| 886 | K886E | 0.69 |
| 887 | T887F | 0.54 |
| 887 | T887R | 0.89 |
| 887 | T887V | 0.78 |
| 888 | V888D | 0.76 |
| 888 | V888G | 0.88 |
| 892 | Y892D | 0.8 |
| 892 | Y892R | 0.57 |
| 895 | R895M | 0.87 |
| 896 | N896M | 0.64 |
| 899 | V899G | 0.74 |
| 901 | T901G | 0.64 |
| 901 | T901V | 0.42 |
| 902 | G902L | 0.67 |
| 906 | I906D | 0.83 |
| 906 | I906S | 0.73 |
| 906 | I906Y | 0.76 |
| 907 | L907Y | 0.8 |
| 908 | P908M | 0.89 |
| 910 | D910C | 0.87 |
| 910 | D910S | 0.89 |
| 911 | V911S | 0.84 |
| 913 | V913Q | 0.71 |
| 913 | V913W | 0.85 |
| 918 | G918W | 0.85 |
| 919 | T919Y | 0.78 |
| 920 | S920C | 0.83 |
| 920 | S920E | 0.68 |
| 920 | S920M | 0.81 |
| 920 | S920R | 0.85 |
| 920 | S920W | 0.8 |
| 921 | D921P | 0.84 |
| 921 | D921Q | 0.74 |
| 922 | R922G | 0.78 |
| 923 | Q923C | 0.81 |
| 923 | Q923W | 0.89 |
| 924 | N924A | 0.86 |
| 924 | N924L | 0.74 |
| 924 | N924P | 0.84 |
| 924 | N924W | 0.86 |
| 925 | V925A | 0.75 |
| 925 | V925S | 0.79 |
| 926 | T926G | 0.66 |
| 926 | T926S | 0.8 |
| 926 | T926V | 0.84 |
| 926 | T926W | 0.8 |
| 927 | W927C | 0.77 |
| 927 | W927G | 0.56 |
| 928 | D928E | 0.86 |
| 928 | D928L | 0.84 |
| 929 | A929C | 0.78 |
| 929 | A929V | 0.81 |
| 930 | V930A | 0.78 |
| 930 | V930K | 0.81 |
| 930 | V930M | 0.87 |
| 931 | S931G | 0.74 |
| 932 | D932F | 0.8 |
| 932 | D932R | 0.83 |
| 932 | D932S | 0.74 |
| 932 | D932T | 0.8 |
| 932 | D932V | 0.7 |
| 933 | D933I | 0.76 |
| 933 | D933S | 0.87 |
| 937 | K937R | 0.79 |
| 937 | K937V | 0.85 |
| 940 | S940M | 0.82 |
| 940 | S940T | 0.58 |
| 940 | S940W | 0.76 |
| 941 | F941W | 0.71 |
| 942 | S942L | 0.77 |
| 942 | S942T | 0.88 |
| 943 | V943H | 0.38 |
| 943 | V943R | 0.25 |
| 944 | A944D | 0.7 |
| 944 | A944G | 0.85 |
| 944 | A944H | 0.62 |
| 944 | A944P | 0.24 |
| 944 | A944R | 0.63 |
| 944 | A944V | 0.83 |
| 945 | G945E | 0.85 |
| 945 | G945P | 0.83 |
| 945 | G945T | 0.78 |
| 946 | T946E | 0.85 |
| 946 | T946G | 0.75 |
| 946 | T946V | 0.34 |
| 947 | V947H | 0.86 |
| 947 | V947M | 0.68 |
| 948 | A948C | 0.58 |
| 948 | A948R | 0.62 |
| 949 | G949F | 0.82 |
| 950 | Q950D | 0.59 |
| 950 | Q950G | 0.76 |
| 950 | Q950M | 0.82 |
| 951 | K951Q | 0.79 |
| 952 | I952H | 0.7 |
| 953 | S953W | 0.69 |
| 954 | V954L | 0.87 |
| 954 | V954S | 0.88 |
| 954 | V954T | 0.72 |
| 955 | R955E | 0.88 |
| 958 | M958D | 0.66 |
| 958 | M958I | 0.72 |
| 958 | M958K | 0.85 |
| 960 | D960G | 0.83 |
| 960 | D960H | 0.73 |
| 960 | D960L | 0.74 |
| 960 | D960W | 0.86 |
| 961 | E961P | 0.85 |
| 962 | I962Q | 0.89 |
| 962 | I962T | 0.89 |
| 963 | G963L | 0.86 |
| 964 | A964C | 0.81 |
| 964 | A964E | 0.89 |
| 964 | A964H | 0.89 |
| 966 | L966G | 0.75 |
| 966 | L966H | 0.69 |
| 966 | L966P | 0.78 |
| 966 | L966T | 0.77 |
| 967 | N967C | 0.83 |
| 967 | N967S | 0.72 |
| 971 | S971E | 0.89 |
| 971 | S971F | 0.87 |
| 971 | S971H | 0.73 |
| 975 | G975D | 0.89 |
| 975 | G975L | 0.83 |
| 975 | G975Q | 0.78 |
| 975 | G975V | 0.9 |
| 976 | T976Y | 0.86 |
| 977 | P977A | 0.86 |
| 978 | A978P | 0.86 |
| 979 | V979R | 0.53 |
| 982 | G982A | 0.8 |
| 982 | G982H | 0.88 |
| 982 | G982M | 0.86 |
| 982 | G982P | 0.78 |
| 982 | G982Q | 0.74 |
| 982 | G982W | 0.88 |
| 984 | R984S | 0.88 |
| 985 | P985E | 0.74 |
| 986 | A986E | 0.85 |
| 986 | A986F | 0.85 |
| 986 | A986K | 0.88 |
| 986 | A986N | 0.76 |
| 986 | A986S | 0.89 |
| 987 | V987I | 0.86 |
| 987 | V987L | 0.9 |
| 987 | V987Q | 0.85 |
| 987 | V987T | 0.83 |
| 988 | L988A | 0.85 |
| 988 | L988C | 0.88 |
| 988 | L988E | 0.77 |
| 988 | L988G | 0.79 |
| 988 | L988H | 0.82 |
| 988 | L988M | 0.87 |
| 988 | L988Q | 0.87 |
| 988 | L988R | 0.86 |
| 988 | L988S | 0.75 |
| 988 | L988Y | 0.86 |
| 989 | P989A | 0.75 |
| 989 | P989C | 0.87 |
| 989 | P989D | 0.89 |
| 989 | P989G | 0.9 |
| 989 | P989H | 0.89 |
| 989 | P989I | 0.82 |
| 989 | P989N | 0.86 |
| 989 | P989Q | 0.84 |
| 990 | D990S | 0.86 |
| 992 | T992E | 0.89 |
| 992 | T992H | 0.87 |
| 992 | T992Y | 0.88 |
| 993 | V993D | 0.89 |
| 993 | V993N | 0.86 |
| 993 | V993S | 0.84 |
| 994 | T994I | 0.85 |
| 994 | T994S | 0.79 |
| 994 | T994V | 0.87 |
| 995 | S995V | 0.79 |
| 996 | A996Q | 0.77 |
| 996 | A996V | 0.76 |
| 997 | N997A | 0.75 |
| 997 | N997C | 0.83 |
| 997 | N997E | 0.78 |
| 997 | N997L | 0.72 |
| 997 | N997S | 0.82 |
| 997 | N997W | 0.87 |
| 997 | N997Y | 0.71 |
| 999 | A999F | 0.9 |
| 999 | A999G | 0.63 |
| 999 | A999L | 0.85 |
| 999 | A999M | 0.79 |
| 999 | A999R | 0.78 |
| 999 | A999S | 0.76 |
| 1000 | V1000C | 0.79 |
| 1000 | V1000L | 0.78 |
| 1000 | V1000M | 0.71 |
| 1000 | V1000N | 0.67 |
| 1000 | V1000P | 0.88 |
| 1001 | D1001G | 0.79 |
| 1001 | D1001K | 0.79 |
| 1001 | D1001M | 0.82 |
| 1001 | D1001Q | 0.85 |
| 1001 | D1001T | 0.77 |
| 1001 | D1001V | 0.77 |
| 1001 | D1001Y | 0.88 |
| 1003 | T1003L | 0.62 |
| 1003 | T1003P | 0.79 |
| 1004 | K1004F | 0.77 |
| 1004 | K1004G | 0.89 |
| 1004 | K1004H | 0.54 |
| 1004 | K1004M | 0.83 |
| 1004 | K1004P | 0.78 |
| 1004 | K1004R | 0.75 |
| 1004 | K1004V | 0.8 |
| 1005 | P1005Q | 0.87 |
| 1006 | A1006I | 0.76 |
| 1009 | V1009G | 0.79 |
| 1009 | V1009S | 0.86 |
| 1012 | T1012E | 0.86 |
| 1012 | T1012Y | 0.85 |
| 1013 | A1013K | 0.87 |
| 1013 | A1013T | 0.88 |
| 1014 | G1014E | 0.87 |
| 1014 | G1014L | 0.8 |
| 1014 | G1014Y | 0.9 |
| 1015 | T1015F | 0.83 |
| 1015 | T1015G | 0.79 |
| 1015 | T1015V | 0.88 |
| 1016 | V1016C | 0.79 |
| 1018 | V1018I | 0.83 |
| 1018 | V1018M | 0.85 |
| 1018 | V1018W | 0.88 |
| 1021 | T1021F | 0.67 |
| 1021 | T1021K | 0.86 |
| 1021 | T1021L | 0.89 |
| 1021 | T1021S | 0.89 |
| 1021 | T1021V | 0.75 |
| 1022 | A1022L | 0.76 |
| 1022 | A1022S | 0.84 |
| 1022 | A1022Y | 0.88 |
| 1023 | T1023D | 0.78 |
| 1023 | T1023M | 0.89 |
| 1023 | T1023Q | 0.88 |
| 1023 | T1023R | 0.85 |
| 1024 | V1024E | 0.81 |
| 1024 | V1024N | 0.76 |
| 1026 | G1026H | 0.86 |
| 1026 | G1026L | 0.72 |
| 1026 | G1026R | 0.58 |
| 1027 | K1027C | 0.8 |
| 1027 | K1027N | 0.76 |
| 1027 | K1027V | 0.85 |
| 1028 | E1028S | 0.88 |
| 1029 | F1029L | 0.84 |
| 1029 | F1029W | 0.85 |
| 1029 | F1029Y | 0.88 |
| 1030 | K1030F | 0.86 |
| 1030 | K1030L | 0.89 |
| 1031 | V1031Y | 0.84 |
| 1033 | A1033G | 0.84 |
| 1033 | A1033S | 0.88 |
| 1034 | T1034W | 0.82 |
| 1037 | V1037F | 0.73 |
| 1037 | V1037P | 0.9 |
| 1037 | V1037Q | 0.83 |
| 1038 | Q1038A | 0.87 |
| 1038 | Q1038K | 0.87 |
| 1039 | R1039S | 0.88 |
| 1040 | S1040A | 0.87 |
| 1040 | S1040N | 0.84 |
| 1040 | S1040R | 0.9 |
| 1040 | S1040W | 0.86 |
| 1043 | T1043N | 0.89 |
| 1045 | G1045S | 0.84 |
| 1046 | S1046I | 0.82 |
| 1047 | S1047D | 0.88 |
| 1050 | G1050L | 0.84 |
| 1050 | G1050S | 0.45 |
| 1050 | G1050V | 0.9 |
| 1052 | A1052M | 0.87 |
| 1052 | A1052P | 0.87 |
| 1054 | R1054N | 0.89 |
| 1057 | Q1057R | 0.5 |
| 1058 | N1058V | 0.62 |
| 1058 | N1058W | 0.84 |
| 1059 | I1059W | 0.69 |
| 1060 | P1060S | 0.82 |
| 1060 | P1060T | 0.72 |
| 1061 | A1061E | 0.82 |
| 1062 | D1062F | 0.83 |
| 1062 | D1062L | 0.66 |
| 1062 | D1062P | 0.87 |
| 1062 | D1062S | 0.87 |
| 1063 | K1063D | 0.71 |
| 1064 | Q1064C | 0.84 |
| 1065 | S1065E | 0.73 |
| 1065 | S1065G | 0.83 |
| 1065 | S1065T | 0.86 |
| 1065 | S1065W | 0.87 |
| 1066 | D1066M | 0.64 |
| 1066 | D1066W | 0.31 |
| 1068 | L1068C | 0.31 |
| 1068 | L1068E | 0.82 |
| 1069 | D1069G | 0.87 |
| 1069 | D1069K | 0.81 |
| 1069 | D1069W | 0.82 |
| 1070 | A1070P | 0.69 |
| 1071 | I1071R | 0.77 |
| 1071 | I1071W | 0.54 |
| 1072 | K1072E | 0.75 |
| 1072 | K1072G | 0.56 |
| 1072 | K1072Q | 0.57 |
| 1073 | D1073L | 0.78 |
| 1073 | D1073M | 0.81 |
| 1073 | D1073P | 0.65 |
| 1074 | G1074I | 0.65 |
| 1075 | S1075C | 0.69 |
| 1075 | S1075I | 0.88 |
| 1075 | S1075V | 0.82 |
| 1076 | T1076C | 0.81 |
| 1076 | T1076H | 0.72 |
| 1076 | T1076Q | 0.79 |
| 1077 | T1077K | 0.9 |
| 1077 | T1077L | 0.88 |
| 1078 | V1078E | 0.88 |
| 1078 | V1078L | 0.77 |
| 1081 | N1081D | 0.84 |
| 1081 | N1081G | 0.89 |
| 1082 | T1082A | 0.82 |
| 1082 | T1082C | 0.81 |
| 1082 | T1082N | 0.88 |
| 1082 | T1082S | 0.83 |
| 1083 | G1083P | 0.84 |
| 1083 | G1083S | 0.65 |
| 1084 | G1084C | 0.82 |
| 1085 | G1085A | 0.85 |
| 1086 | A1086Q | 0.79 |
| 1086 | A1086T | 0.81 |
| 1087 | N1087V | 0.82 |
| 1087 | N1087W | 0.75 |
| 1089 | S1089C | 0.67 |
| 1089 | S1089E | 0.89 |
| 1089 | S1089R | 0.72 |
| 1090 | A1090G | 0.78 |
| 1090 | A1090I | 0.85 |
| 1090 | A1090K | 0.84 |
| 1091 | W1091A | 0.53 |
| 1091 | W1091E | 0.61 |
| 1091 | W1091G | 0.69 |
| 1091 | W1091H | 0.73 |
| 1091 | W1091T | 0.68 |
| 1091 | W1091V | 0.54 |
| 1092 | T1092A | 0.86 |
| 1092 | T1092E | 0.85 |
| 1092 | T1092G | 0.69 |
| 1092 | T1092K | 0.9 |
| 1092 | T1092Q | 0.59 |
| 1092 | T1092S | 0.75 |
| 1092 | T1092V | 0.77 |
| 1093 | N1093T | 0.7 |
| 1093 | N1093V | 0.76 |
| 1095 | A1095R | 0.89 |
| 1096 | Y1096A | 0.73 |
| 1096 | Y1096H | 0.87 |
| 1096 | Y1096R | 0.88 |
| 1097 | S1097D | 0.87 |
| 1097 | S1097L | 0.74 |
| 1097 | S1097W | 0.36 |
| 1099 | A1099C | 0.74 |
| 1099 | A1099F | 0.81 |
| 1100 | G1100D | 0.87 |
| 1100 | G1100M | 0.69 |
| 1100 | G1100T | 0.76 |
| 1101 | H1101L | 0.85 |
| 1102 | N1102E | 0.59 |
| 1102 | N1102F | 0.81 |
| 1102 | N1102K | 0.72 |
| 1102 | N1102L | 0.85 |
| 1102 | N1102R | 0.86 |
| 1103 | T1103H | 0.88 |
| 1104 | A1104I | 0.89 |
| 1104 | A1104K | 0.88 |
| 1104 | A1104R | 0.58 |
| 1106 | I1106T | 0.67 |
| 1106 | I1106V | 0.83 |
| 1107 | T1107C | 0.83 |
| 1107 | T1107M | 0.87 |
| 1107 | T1107R | 0.75 |
| 1107 | T1107S | 0.54 |
| 1108 | F1108D | 0.69 |
| 1108 | F1108K | 0.87 |
| 1108 | F1108L | 0.68 |
| 1108 | F1108T | 0.73 |
| 1108 | F1108W | 0.81 |
| 1113 | E1113D | 0.79 |
| 1114 | Q1114I | 0.87 |
| 1115 | Q1115T | 0.81 |
| 1115 | Q1115W | 0.86 |
| 1116 | L1116V | 0.81 |
| 1118 | Q1118A | 0.85 |
| 1118 | Q1118S | 0.82 |
| 1118 | Q1118T | 0.88 |
| 1119 | I1119G | 0.71 |
| 1120 | V1120T | 0.76 |
| 1121 | M1121V | 0.61 |
| 1121 | M1121Y | 0.87 |
| 1122 | Y1122A | 0.86 |
| 1122 | Y1122C | 0.9 |
| 1122 | Y1122I | 0.85 |
| 1122 | Y1122K | 0.71 |
| 1122 | Y1122R | 0.8 |
| 1122 | Y1122V | 0.72 |
| 1122 | Y1122W | 0.85 |
| 1123 | F1123H | 0.89 |
| 1123 | F1123T | 0.78 |
| 1124 | F1124E | 0.88 |
| 1124 | F1124R | 0.85 |
| 1125 | R1125E | 0.79 |
| 1125 | R1125T | 0.85 |
| 1126 | D1126H | 0.68 |
| 1126 | D1126K | 0.83 |
| 1126 | D1126L | 0.77 |
| 1126 | D1126R | 0.85 |
| 1127 | S1127F | 0.75 |
| 1127 | S1127K | 0.77 |
| 1127 | S1127M | 0.87 |
| 1127 | S1127T | 0.88 |
| 1127 | S1127W | 0.76 |
| 1128 | N1128C | 0.86 |
| 1128 | N1128R | 0.7 |
| 1128 | N1128S | 0.74 |
| 1128 | N1128T | 0.83 |
| 1128 | N1128W | 0.73 |
| 1129 | A1129L | 0.77 |
| 1129 | A1129N | 0.87 |
| 1129 | A1129Q | 0.65 |
| 1129 | A1129V | 0.65 |
| 1130 | V1130G | 0.58 |
| 1130 | V1130P | 0.87 |
| 1130 | V1130S | 0.77 |
| 1131 | R1131A | 0.71 |
| 1131 | R1131N | 0.9 |
| 1131 | R1131Q | 0.88 |
| 1131 | R1131S | 0.75 |
| 1132 | F1132E | 0.73 |
| 1132 | F1132K | 0.84 |
| 1132 | F1132T | 0.89 |
| 1133 | P1133Q | 0.87 |
| 1133 | P1133V | 0.84 |
| 1134 | D1134E | 0.89 |
| 1134 | D1134G | 0.74 |
| 1134 | D1134L | 0.64 |
| 1135 | A1135M | 0.88 |
| 1135 | A1135S | 0.61 |
| 1135 | A1135Y | 0.76 |
| 1136 | G1136A | 0.69 |
| 1136 | G1136E | 0.51 |
| 1136 | G1136Q | 0.82 |
| 1136 | G1136T | 0.88 |
| 1137 | K1137A | 0.84 |
| 1137 | K1137C | 0.61 |
| 1137 | K1137L | 0.7 |
| 1137 | K1137Q | 0.69 |
| 1139 | K1139A | 0.88 |
| 1139 | K1139L | 0.8 |
| 1139 | K1139R | 0.9 |
| 1139 | K1139T | 0.86 |
| 1140 | I1140A | 0.86 |
| 1140 | I1140G | 0.64 |
| 1140 | I1140L | 0.61 |
| 1140 | I1140M | 0.85 |
| 1140 | I1140T | 0.81 |
| 1141 | Q1141C | 0.86 |
| 1141 | Q1141P | 0.6 |
| 1141 | Q1141W | 0.78 |
| 1142 | I1142S | 0.89 |
| 1142 | I1142W | 0.87 |
| 1144 | A1144C | 0.68 |
| 1144 | A1144E | 0.87 |
| 1146 | G1146C | 0.88 |
| 1146 | G1146D | 0.78 |
| 1146 | G1146L | 0.8 |
| 1147 | K1147A | 0.84 |
| 1147 | K1147G | 0.89 |
| 1147 | K1147T | 0.76 |
| 1147 | K1147V | 0.7 |
| 1148 | N1148K | 0.88 |
| 1148 | N1148W | 0.86 |
| 1149 | W1149I | 0.66 |
| 1149 | W1149N | 0.8 |
| 1149 | W1149Q | 0.79 |
| 1149 | W1149T | 0.9 |
| 1149 | W1149Y | 0.89 |
| 1150 | T1150G | 0.81 |
| 1151 | D1151G | 0.84 |
| 1151 | D1151R | 0.38 |
| 1151 | D1151T | 0.81 |
| 1153 | A1153K | 0.83 |
| 1154 | A1154C | 0.69 |
| 1154 | A1154G | 0.82 |
| 1154 | A1154S | 0.85 |
| 1155 | T1155E | 0.85 |
| 1155 | T1155L | 0.78 |
| 1155 | T1155Q | 0.86 |
| 1155 | T1155R | 0.87 |
| 1157 | T1157V | 0.85 |
| 1157 | T1157W | 0.9 |
| 1158 | I1158R | 0.76 |
| 1159 | A1159C | 0.85 |
| 1159 | A1159P | 0.9 |
| 1159 | A1159R | 0.76 |
| 1159 | A1159V | 0.75 |
| 1160 | A1160K | 0.82 |
| 1160 | A1160L | 0.87 |
| 1160 | A1160Q | 0.8 |
| 1160 | A1160S | 0.82 |
| 1161 | Q1161A | 0.8 |
| 1162 | E1162D | 0.86 |
| 1162 | E1162F | 0.82 |
| 1162 | E1162I | 0.75 |
| 1162 | E1162N | 0.82 |
| 1162 | E1162T | 0.87 |
| 1162 | E1162Y | 0.73 |
| 1165 | E1165D | 0.78 |
| 1165 | E1165H | 0.82 |
| 1165 | E1165L | 0.65 |
| 1165 | E1165R | 0.81 |
| 1165 | E1165W | 0.87 |
| 1166 | R1166D | 0.85 |
| 1166 | R1166K | 0.82 |
| 1167 | V1167C | 0.85 |
| 1167 | V1167L | 0.89 |
| 1167 | V1167P | 0.82 |
| 1168 | K1168Q | 0.8 |
| 1168 | K1168W | 0.61 |
| 1169 | P1169R | 0.86 |
| 1170 | Y1170E | 0.65 |
| 1170 | Y1170Q | 0.88 |
| 1170 | Y1170R | 0.83 |
| 1171 | T1171A | 0.68 |
| 1171 | T1171G | 0.69 |
| 1171 | T1171M | 0.87 |
| 1171 | T1171Q | 0.87 |
| 1171 | T1171R | 0.89 |
| 1171 | T1171S | 0.83 |
| 1172 | Y1172D | 0.87 |
| 1172 | Y1172E | 0.8 |
| 1172 | Y1172I | 0.81 |
| 1172 | Y1172K | 0.77 |
| 1172 | Y1172L | 0.88 |
| 1172 | Y1172V | 0.84 |
| 1173 | D1173A | 0.78 |
| 1173 | D1173E | 0.88 |
| 1173 | D1173F | 0.8 |
| 1173 | D1173K | 0.8 |
| 1173 | D1173L | 0.75 |
| 1173 | D1173P | 0.86 |
| 1173 | D1173R | 0.83 |
| 1173 | D1173W | 0.84 |
| 1174 | F1174P | 0.8 |
| 1174 | F1174Q | 0.89 |
| 1174 | F1174R | 0.89 |
| 1174 | F1174S | 0.84 |
| 1174 | F1174T | 0.8 |
| 1174 | F1174V | 0.7 |
| 1174 | F1174W | 0.79 |
| 1175 | A1175G | 0.9 |
| 1175 | A1175I | 0.81 |
| 1175 | A1175N | 0.58 |
| 1175 | A1175Q | 0.68 |
| 1175 | A1175V | 0.72 |
| 1175 | A1175Y | 0.76 |
| 1177 | V1177N | 0.68 |
| 1177 | V1177P | 0.84 |
| 1177 | V1177S | 0.87 |
| 1177 | V1177T | 0.65 |
| 1179 | A1179P | 0.88 |
| 1181 | F1181L | 0.85 |
| 1190 | D1190S | 0.89 |
| 1191 | T1191E | 0.77 |
| 1192 | T1192H | 0.62 |
| 1192 | T1192P | 0.88 |
| 1194 | P1194A | 0.85 |
| 1194 | P1194G | 0.89 |
| 1195 | S1195G | 0.88 |
| 1198 | V1198E | 0.72 |
| 1199 | C1199T | 0.51 |
| 1200 | A1200V | 0.7 |
| 1200 | A1200W | 0.67 |
| 1202 | L1202A | 0.65 |
| 1202 | L1202C | 0.82 |
| 1203 | T1203E | 0.89 |
| 1210 | A1210E | 0.7 |
| 1213 | K1213W | 0.82 |
| 1214 | F1214R | 0.88 |
| 1215 | V1215K | 0.89 |
| 1215 | V1215S | 0.86 |
| 1217 | N1217D | 0.88 |
| 1217 | N1217F | 0.79 |
| 1219 | S1219A | 0.5 |
| 1219 | S1219E | 0.84 |
| 1219 | S1219F | 0.6 |
| 1220 | A1220L | 0.69 |
| 1220 | A1220R | 0.76 |
| 1220 | A1220V | 0.85 |
| 1221 | A1221D | 0.75 |
| 1221 | A1221G | 0.84 |
| 1221 | A1221L | 0.86 |
| 1222 | L1222C | 0.83 |
| 1223 | S1223C | 0.83 |
| 1223 | S1223F | 0.75 |
| 1223 | S1223G | 0.85 |
| 1223 | S1223K | 0.79 |
| 1223 | S1223L | 0.77 |
| 1223 | S1223V | 0.38 |
| 1224 | S1224A | 0.65 |
| 1224 | S1224D | 0.72 |
| 1224 | S1224G | 0.66 |
| 1224 | S1224L | 0.8 |
| 1224 | S1224M | 0.83 |
| 1224 | S1224R | 0.87 |
| 1225 | L1225C | 0.75 |
| 1225 | L1225D | 0.9 |
| 1225 | L1225F | 0.79 |
| 1225 | L1225G | 0.83 |
| 1225 | L1225P | 0.87 |
| 1225 | L1225T | 0.82 |
| 1225 | L1225W | 0.65 |
| 1226 | T1226A | 0.75 |
| 1226 | T1226G | 0.75 |
| 1226 | T1226M | 0.86 |
| 1226 | T1226R | 0.83 |
| 1226 | T1226S | 0.66 |
| 1226 | T1226V | 0.63 |
| 1226 | T1226Y | 0.89 |
| 1227 | V1227A | 0.71 |
| 1227 | V1227E | 0.83 |
| 1227 | V1227G | 0.89 |
| 1227 | V1227L | 0.76 |
| 1227 | V1227S | 0.76 |
| 1228 | N1228A | 0.87 |
| 1228 | N1228D | 0.63 |
| 1228 | N1228F | 0.58 |
| 1228 | N1228K | 0.89 |
| 1228 | N1228L | 0.85 |
| 1228 | N1228T | 0.73 |
| 1229 | G1229A | 0.86 |
| 1229 | G1229Q | 0.83 |
| 1229 | G1229S | 0.89 |
| 1230 | T1230F | 0.85 |
| 1230 | T1230I | 0.62 |
| 1230 | T1230L | 0.87 |
| 1230 | T1230P | 0.86 |
| 1230 | T1230R | 0.82 |
| 1230 | T1230S | 0.77 |
| 1231 | K1231F | 0.58 |
| 1231 | K1231L | 0.73 |
| 1231 | K1231M | 0.78 |
| 1231 | K1231P | 0.63 |
| 1231 | K1231S | 0.88 |
| 1231 | K1231W | 0.9 |
| 1232 | V1232K | 0.68 |
| 1232 | V1232S | 0.86 |
| 1232 | V1232T | 0.86 |
| 1232 | V1232W | 0.88 |
| 1233 | S1233P | 0.56 |
| 1233 | S1233W | 0.63 |
| 1234 | D1234G | 0.88 |
| 1234 | D1234K | 0.71 |
| 1234 | D1234R | 0.78 |
| 1235 | S1235L | 0.83 |
| 1236 | V1236I | 0.79 |
| 1236 | V1236R | 0.8 |
| 1237 | L1237D | 0.61 |
| 1237 | L1237E | 0.73 |
| 1237 | L1237R | 0.87 |
| 1237 | L1237W | 0.82 |
| 1238 | A1238K | 0.88 |
| 1238 | A1238N | 0.73 |
| 1238 | A1238P | 0.73 |
| 1238 | A1238S | 0.81 |
| 1238 | A1238T | 0.86 |
| 1239 | A1239D | 0.74 |
| 1239 | A1239P | 0.73 |
| 1240 | G1240L | 0.88 |
| 1240 | G1240N | 0.78 |
| 1240 | G1240S | 0.86 |
| 1240 | G1240T | 0.81 |
| 1240 | G1240W | 0.62 |
| 1241 | S1241D | 0.76 |
| 1241 | S1241G | 0.87 |
| 1241 | S1241I | 0.84 |
| 1241 | S1241L | 0.86 |
| 1241 | S1241M | 0.88 |
| 1241 | S1241P | 0.89 |
| 1242 | Y1242C | 0.9 |
| 1242 | Y1242E | 0.67 |
| 1242 | Y1242K | 0.88 |
| 1242 | Y1242R | 0.67 |
| 1242 | Y1242S | 0.87 |
| 1242 | Y1242W | 0.9 |
| 1243 | N1243L | 0.89 |
| 1243 | N1243M | 0.66 |
| 1243 | N1243P | 0.8 |
| 1243 | N1243Q | 0.86 |
| 1243 | N1243S | 0.9 |
| 1243 | N1243T | 0.56 |
| 1243 | N1243V | 0.76 |
| 1243 | N1243W | 0.78 |
| 1244 | T1244A | 0.73 |
| 1244 | T1244D | 0.84 |
| 1244 | T1244E | 0.71 |
| 1244 | T1244G | 0.83 |
| 1244 | T1244L | 0.89 |
| 1244 | T1244Q | 0.56 |
| 1244 | T1244S | 0.83 |
| 1244 | T1244V | 0.86 |
| 1244 | T1244W | 0.76 |
| 1246 | A1246F | 0.81 |
| 1246 | A1246M | 0.51 |
| 1246 | A1246N | 0.79 |
| 1246 | A1246P | 0.79 |
| 1246 | A1246Q | 0.77 |
| 1246 | A1246R | 0.8 |
| 1246 | A1246S | 0.81 |
| 1246 | A1246T | 0.82 |
| 1247 | I1247A | 0.83 |
| 1247 | I1247G | 0.71 |
| 1247 | I1247M | 0.88 |
| 1247 | I1247Q | 0.72 |
| 1247 | I1247S | 0.77 |
| 1247 | I1247T | 0.86 |
| 1247 | I1247V | 0.89 |
| 1247 | I1247W | 0.74 |
| 1248 | I1248A | 0.84 |
| 1248 | I1248G | 0.64 |
| 1248 | I1248K | 0.83 |
| 1248 | I1248L | 0.74 |
| 1248 | I1248R | 0.73 |
| 1248 | I1248S | 0.72 |
| 1248 | I1248Y | 0.9 |
| 1249 | A1249E | 0.83 |
| 1249 | A1249G | 0.77 |
| 1249 | A1249H | 0.69 |
| 1249 | A1249I | 0.69 |
| 1249 | A1249R | 0.74 |
| 1249 | A1249T | 0.72 |
| 1249 | A1249V | 0.67 |
| 1250 | D1250I | 0.6 |
| 1250 | D1250K | 0.7 |
| 1250 | D1250S | 0.54 |
| 1250 | D1250T | 0.7 |
| 1250 | D1250W | 0.54 |
| 1250 | D1250Y | 0.64 |
| 1251 | V1251I | 0.81 |
| 1251 | V1251T | 0.78 |
| 1251 | V1251W | 0.79 |
| 1252 | K1252D | 0.66 |
| 1252 | K1252G | 0.85 |
| 1252 | K1252V | 0.79 |
| 1252 | K1252W | 0.74 |
| 1253 | A1253P | 0.87 |
| 1253 | A1253V | 0.81 |
| 1254 | E1254F | 0.83 |
| 1254 | E1254G | 0.87 |
| 1254 | E1254H | 0.87 |
| 1254 | E1254L | 0.87 |
| 1254 | E1254R | 0.83 |
| 1254 | E1254V | 0.71 |
| 1255 | G1255M | 0.71 |
| 1255 | G1255S | 0.83 |
| 1255 | G1255V | 0.89 |
| 1256 | E1256G | 0.85 |
| 1256 | E1256M | 0.78 |
| 1256 | E1256N | 0.73 |
| 1256 | E1256R | 0.83 |
| 1256 | E1256V | 0.74 |
| 1256 | E1256W | 0.77 |
| 1257 | G1257F | 0.8 |
| 1257 | G1257K | 0.83 |
| 1257 | G1257L | 0.75 |
| 1257 | G1257Q | 0.76 |
| 1257 | G1257R | 0.85 |
| 1257 | G1257W | 0.88 |
| 1258 | N1258C | 0.85 |
| 1258 | N1258G | 0.86 |
| 1258 | N1258H | 0.61 |
| 1258 | N1258K | 0.83 |
| 1258 | N1258S | 0.77 |
| 1259 | A1259L | 0.89 |
| 1259 | A1259W | 0.89 |
| 1261 | V1261I | 0.8 |
| 1261 | V1261L | 0.79 |
| 1261 | V1261P | 0.89 |
| 1261 | V1261Q | 0.85 |
| 1261 | V1261T | 0.85 |
| 1262 | T1262A | 0.74 |
| 1262 | T1262Q | 0.77 |
| 1263 | V1263E | 0.79 |
| 1263 | V1263G | 0.81 |
| 1263 | V1263Q | 0.83 |
| 1263 | V1263R | 0.72 |
| 1263 | V1263W | 0.67 |
| 1264 | L1264A | 0.83 |
| 1264 | L1264E | 0.83 |
| 1264 | L1264S | 0.87 |
| 1264 | L1264Y | 0.9 |
| 1265 | P1265C | 0.9 |
| 1265 | P1265K | 0.87 |
| 1265 | P1265L | 0.71 |
| 1265 | P1265R | 0.64 |
| 1265 | P1265S | 0.83 |
| 1265 | P1265V | 0.87 |
| 1266 | A1266F | 0.81 |
| 1266 | A1266L | 0.82 |
| 1266 | A1266P | 0.74 |
| 1266 | A1266S | 0.87 |
| 1266 | A1266V | 0.78 |
| 1267 | H1267A | 0.82 |
| 1267 | H1267E | 0.75 |
| 1267 | H1267F | 0.87 |
| 1269 | N1269A | 0.44 |
| 1269 | N1269E | 0.82 |
| 1269 | N1269K | 0.58 |
| 1269 | N1269R | 0.76 |
| 1269 | N1269S | 0.78 |
| 1269 | N1269T | 0.82 |
| 1269 | N1269W | 0.89 |
| 1270 | V1270D | 0.77 |
| 1270 | V1270E | 0.8 |
| 1270 | V1270G | 0.81 |
| 1270 | V1270I | 0.83 |
| 1270 | V1270L | 0.89 |
| 1270 | V1270T | 0.81 |
| 1270 | V1270W | 0.83 |
| 1271 | I1271A | 0.84 |
| 1271 | I1271H | 0.71 |
| 1271 | I1271Q | 0.89 |
| 1272 | R1272E | 0.82 |
| 1272 | R1272F | 0.81 |
| 1272 | R1272M | 0.79 |
| 1272 | R1272P | 0.8 |
| 1272 | R1272V | 0.72 |
| 1274 | I1274M | 0.89 |
| 1275 | T1275L | 0.8 |
| 1276 | E1276W | 0.86 |
| 1279 | D1279G | 0.71 |
| 1279 | D1279T | 0.77 |
| 1279 | D1279V | 0.84 |
| 1280 | H1280V | 0.87 |
| 1280 | H1280W | 0.84 |
| 1281 | V1281F | 0.9 |
| 1281 | V1281S | 0.87 |
| 1282 | T1282D | 0.8 |
| 1282 | T1282L | 0.88 |
| 1282 | T1282V | 0.88 |
| 1283 | R1283A | 0.78 |
| 1283 | R1283D | 0.76 |
| 1283 | R1283E | 0.77 |
| 1283 | R1283P | 0.84 |
| 1283 | R1283W | 0.73 |
| 1285 | T1285E | 0.84 |
| 1285 | T1285F | 0.72 |
| 1285 | T1285G | 0.84 |
| 1285 | T1285R | 0.85 |
| 1285 | T1285Y | 0.85 |
| 1286 | F1286S | 0.78 |
| 1286 | F1286T | 0.89 |
| 1287 | T1287K | 0.81 |
| 1287 | T1287M | 0.82 |
| 1287 | T1287Q | 0.85 |
| 1287 | T1287R | 0.76 |
| 1288 | I1288F | 0.9 |
| 1289 | N1289A | 0.74 |
| 1289 | N1289Q | 0.81 |
| 1289 | N1289T | 0.79 |
| 1290 | L1290A | 0.75 |
| 1290 | L1290G | 0.88 |
| 1290 | L1290R | 0.75 |
| 1290 | L1290V | 0.87 |
| 1290 | L1290W | 0.83 |
| 1290 | G1291K | 0.89 |
| 1291 | G1291P | 0.68 |
| 1291 | G1291V | 0.7 |
| 1291 | G1291W | 0.82 |
| 1291 | G1291Y | 0.68 |
| 1292 | T1292G | 0.87 |
| 1292 | T1292L | 0.87 |
| 1292 | T1292Y | 0.69 |
| 1293 | E1293G | 0.86 |
| 1293 | E1293S | 0.79 |
| 1293 | E1293V | 0.78 |
| 1294 | Q1294L | 0.83 |
| 1294 | Q1294P | 0.78 |
| 1294 | Q1294W | 0.77 |
| 1296 | F1296G | 0.89 |
| 1296 | F1296I | 0.88 |
| 1297 | P1297F | 0.79 |
| 1298 | A1298Y | 0.87 |
| 1301 | D1301I | 0.88 |
| 1304 | D1304A | 0.78 |

1. A lactase variant having an amino acid sequence which is at least 80% identical to SEQ ID NO: 1 which comprises an alteration, preferably a substitution, at one or more positions corresponding to positions 2, 3, 4, 5, 6, 7, 9, 10, 11, 12, 13, 14, 15, 16, 17, 21, 22, 23, 24, 25, 26, 27, 28, 29, 31, 32, 33, 34, 35, 36, 37, 38, 39, 40, 41, 42, 43, 44, 45, 47, 48, 49, 51, 54, 55, 57, 58, 59, 60, 61, 62, 63, 64, 66, 67, 68, 70, 71, 73, 74, 75, 76, 77, 78, 79, 80, 81, 83, 85, 86, 87, 88, 89, 90, 91, 92, 93, 94, 95, 96, 97, 98, 100, 101, 102, 104, 105, 106, 107, 108, 109, 110, 111, 112, 113, 114, 115, 116, 117, 118, 119, 120, 121, 122, 123, 124, 125, 126, 128, 129, 130, 131, 132, 133, 135, 136, 137, 138, 139, 140, 141, 142, 146, 148, 150, 151, 153, 154, 155, 157, 158, 159, 160, 161, 162, 164, 165, 166, 167, 168, 169, 170, 171, 172, 173, 174, 175, 176, 177, 178, 179, 180, 181, 182, 183, 184, 185, 186, 187, 188, 189, 190, 191, 193, 194, 195, 196, 197, 198, 199, 201, 202, 203, 204, 206, 207, 208, 209, 210, 211, 212, 213, 214, 215, 216, 217, 218, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 254, 255, 256, 257, 258, 259, 260, 261, 262, 263, 265, 267, 268, 269, 271, 272, 273, 274, 275, 277, 278, 279, 280, 281, 282, 283, 284, 285, 286, 287, 288, 291, 292, 293, 294, 295, 296, 297, 298, 299, 301, 302, 303, 304, 305, 306, 307, 308, 310, 311, 312, 314, 315, 316, 317, 318, 320, 321, 322, 324, 325, 326, 328, 329, 331, 335, 337, 338, 340, 341, 342, 343, 344, 345, 346, 347, 348, 349, 350, 351, 352, 353, 354, 355, 357, 359, 360, 362, 363, 364, 365, 366, 367, 368, 369, 370, 371, 372, 373, 374, 375, 376, 377, 378, 379, 380, 381, 383, 386, 387, 388, 389, 390, 391, 392, 393, 394, 395, 396, 398, 399, 400, 401, 402, 403, 404, 406, 407, 408, 409, 410, 411, 412, 413, 414, 415, 416, 417, 418, 419, 421, 422, 423, 424, 425, 426, 427, 428, 429, 430, 431, 432, 433, 434, 435, 436, 437, 438, 439, 440, 441, 442, 443, 444, 445, 446, 448, 449, 450, 451, 452, 453, 454, 455, 456, 457, 459, 461, 462, 463, 464, 465, 466, 467, 469, 470, 471, 472, 473, 474, 475, 476, 477, 478, 479, 480, 485, 487, 488, 491, 492, 493, 494, 495, 496, 497, 498, 499, 500, 501, 502, 503, 504, 505, 506, 507, 508, 509, 510, 511, 512, 514, 516, 517, 518, 519, 520, 521, 524, 525, 526, 527, 528, 529, 530, 531, 534, 535, 537, 538, 539, 540, 542, 543, 545, 546, 547, 548, 549, 550, 551, 553, 554, 555, 556, 557, 558, 559, 560, 561, 563, 564, 565, 567, 570, 571, 572, 573, 574, 575, 576, 577, 578, 579, 580, 581, 582, 583, 584, 585, 586, 587, 588, 589, 590, 591, 592, 593, 594, 595, 597, 598, 600, 604, 605, 606, 608, 609, 610, 611, 612, 613, 631, 633, 634, 635, 636, 637, 638, 639, 640, 641, 642, 643, 644, 645, 646, 647, 648, 649, 650, 651, 652, 653, 654, 655, 656, 657, 659, 661, 662, 669, 670, 672, 673, 674, 675, 676, 677, 678, 679, 680, 681, 682, 683, 684, 685, 686, 687, 688, 689, 690, 693, 694, 695, 696, 697, 698, 700, 701, 703, 704, 705, 706, 708, 709, 710, 711, 712, 713, 714, 715, 718, 720, 721, 723, 724, 725, 726, 727, 728, 729, 730, 731, 732, 733, 734, 735, 736, 737, 738, 739, 740, 741, 742, 743, 744, 745, 746, 747, 748, 750, 751, 752, 753, 755, 756, 757, 758, 759, 760, 761, 762, 765, 766, 768, 769, 770, 771, 772, 773, 774, 775, 776, 777, 778, 779, 780, 781, 782, 783, 784, 785, 786, 787, 788, 789, 790, 791, 792, 793, 794, 795, 796, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 840, 841, 842, 843, 844, 845, 846, 847, 848, 849, 850, 851, 852, 853, 854, 855, 857, 858, 859, 860, 861, 862, 863, 864, 865, 866, 870, 871, 872, 873, 874, 875, 877, 879, 880, 881, 882, 883, 884, 885, 886, 887, 888, 889, 893, 894, 895, 896, 897, 898, 899, 900, 901, 902, 903, 904, 905, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 973, 974, 975, 976, 977, 978, 979, 980, 981, 984, 985, 986, 987, 988, 989, 990, 991, 992, 993, 994, 995, 996, 997, 998, 999, 1000, 1002, 1003, 1004, 1005, 1006, 1007, 1008, 1009, 1010, 1011, 1012, 1013, 1014, 1015, 1016, 1017, 1018, 1021, 1022, 1023, 1024, 1026, 1027, 1028, 1029, 1030, 1031, 1033, 1034, 1035, 1036, 1037, 1038, 1039, 1040, 1041, 1042, 1043, 1044, 1045, 1046, 1047, 1048, 1050, 1051, 1052, 1053, 1055, 1057, 1058, 1059, 1060, 1062, 1063, 1064, 1065, 1066, 1067, 1068, 1070, 1071, 1072, 1073, 1074, 1075, 1076, 1077, 1078, 1079, 1081, 1082, 1083, 1084, 1085, 1086, 1087, 1088, 1089, 1090, 1091, 1092, 1093, 1094, 1095, 1096, 1097, 1098, 1099, 1100, 1101, 1102, 1103, 1104, 1105, 1106, 1107, 1108, 1109, 1111, 1113, 1114, 1115, 1116, 1117, 1118, 1119, 1120, 1121, 1122, 1123, 1124, 1125, 1126, 1127, 1128, 1129, 1130, 1131, 1132, 1133, 1134, 1135, 1136, 1137, 1138, 1139, 1140, 1141, 1142, 1143, 1144, 1145, 1146, 1147, 1148, 1149, 1150, 1151, 1152, 1153, 1154, 1155, 1157, 1158, 1159, 1161, 1162, 1166, 1167, 1168, 1169, 1170, 1171, 1172, 1173, 1174, 1175, 1177, 1178, 1179, 1180, 1181, 1182, 1184, 1185, 1186, 1188, 1189, 1190, 1191, 1192, 1193, 1194, 1195, 1197, 1198, 1199, 1202, 1203, 1204, 1205, 1208, 1209, 1210, 1211, 1213, 1214, 1215, 1217, 1218, 1219, 1220, 1221, 1222, 1223, 1224, 1225, 1226, 1227, 1228, 1230, 1231, 1232, 1233, 1234, 1235, 1236, 1237, 1238, 1239, 1240, 1241, 1242, 1243, 1244, 1246, 1247, 1248, 1249, 1250, 1251, 1252, 1253, 1254, 1255, 1256, 1257, 1258, 1259, 1261, 1262, 1263, 1264, 1265, 1266, 1267, 1269, 1270, 1271, 1272, 1273, 1274, 1275, 1276, 1277, 1278, 1279, 1280, 1281, 1282, 1283, 1284, 1285, 1286, 1287, 1288, 1289, 1290, 1291, 1292, 1293, 1294, 1295, 1296, 1297, 1298, 1301, 1302, 1303 or 1304 of the polypeptide of SEQ ID NO: 1.

2. The lactase variant of claim 1, which comprises one or more of the following substitutions, wherein each position corresponds to a position in SEQ ID NO: 1: E2D, E2G, E2Q, E2V, D3A, D3H, D3I, D3N, D3S, D3V, D3W, A4C, A4G, A4H, A4I, A4L, A4M, A4P, T5A, T5D, T5F, T5K, T5R, T5S, T5V, R6A, R6D, R6F, R6G, R6H, R6L, R6M, R6P, R6S, R6W, S7A, S7D, S7I, S7L, S7N, S7P, S7T, S7W, S9G, S9H, S9P, S9R, S9W, T10G, T10K, T10L, T10P, T10R, T10S, T10W, T11L, T11P, Q12L, Q12R, Q12V, Q12Y, M13C, M13D, M13E, M13F, M13H, M13K, M13R, M13W, S14A, S14G, S14H, S14L, S14T, S14V, S14Y, S15C, S15F, S15I, S15K, S15L, S15P, S15R, S15T, S15V, S15W, S15Y, T16A, T16C, T16I, T16N, T16S, P17A, P17C, P17D, P17E, P17I, P17L, P17N, P17R, P17S, P17T, Y21A, Y21C, Y21D, Y21F, Y21G, Y21H, Y21M, Y21P, Y21R, Y21T, S22A, S22E, S22F, S22G, S22L, S22M, S22N, S22R, S22T, S22W, S23A, S23C, S23D, S23L, S23M, S23R, A24F, A24G, A24L, A24R, A24T, A24W, V25D, V25E, V25F, V25G, V25H, V25K, V25L, V25M, V25Q, V25R, V25S, V25T, V25W, D26C, D26I, D26L, D26M, D26T, D26V, S27A, S27C, S27F, S27G, S27H, S27P, S27Y, K28C, K28G, K28I, K28L, K28R, K28S, K28V, K28W, Q29D, Q29F, Q29G, Q29L, Q29M, Q29R, Q29S, Q29V, Q29W, R31E, R31G, R31I, R31M, R31V, T32M, T32Q, T32R, T32S, S33C, S33E, S33H, S33K, S33N, S33Q, S33R, S33V, D34C, D34E, D34F, D34G, D34H, D34L, D34S, D34W, D34Y, F35A, F35C, F35E, F35G, F35K, F35N, F35T, F35V, D36H, D36Q, A37N, A37Q, N38C, N38G, N38S, W39G, W39S, K40C, K40D, K40F, K40G, K40I, K40M, K40N, K40P, K40W, F41A, F41C, F41G, F41I, F41Q, F41S, F41Y, M42E, M42N, M42T, L43A, L43C, L43G, L43I, L43S, L43T, L43V, S44C, S44M, S44N, S44Y, D45A, D45L, D45P, D45V, V47K, V47R, Q48S, A49C, A49D, A49H, A49R, A49S, A49T, A49V, D51G, D51I, D51K, D51M, D51P, D51V, F54M, F54S, D55C, D55F, D55G, D55H, D55M, D55N, D55P, D55S, D55V, S57A, S57C, S57E, S57G, A58D, A58G, A58I, A58M, A58N, A58Q, A58R, A58T, W59D, W59I, W59K, W59L, W59N, W59P, W59V, Q60A, Q60C, Q60E, Q60F, Q60G, Q60K, Q60L, Q60M, Q60R, Q60S, Q60V, Q60Y, Q61K, Q61P, Q61S, V62G, V62N, V62S, V62T, V62W, D63G, D63L, D63P, D63S, D63V, L64E, L64G, H66C, H66L, H66R, H66T, H66W, H66Y, D67E, Y68P, Y68V, I70A, I70H, I70K, I70P, I70R, T71C, T71E, T71G, T71H, T71K, T71L, T71N, T71P, T71Q, T71R, T71S, K73A, K73D, K73F, K73G, K73Q, K73V, Y74G, Y74K, Y74T, Y74W, S75G, S75L, S75Q, S75R, S75V, Q76G, Q76I, Q76K, Q76M, Q76P, Q76S, Q76V, Q76Y, S77C, S77D, S77E, S77F, S77G, S77H, S77I, S77K, S77L, S77M, S77R, S77T, S77V, S77W, S77Y, N78C, N78E, N78F, N78K, N78Q, N78R, N78S, N78T, E79H, E79Q, E79S, E79T, E79W, A80K, E81A, E81Q, A83E, A83T, L85A, L85C, L85D, L85F, L85M, L85N, L85S, L85V, L85W, P86E, P86G, P86H, P86N, P86Q, P86R, P86V, P86W, P86Y, G87A, G87D, G87E, G87N, G87Q, G88A, G88F, G88I, G88M, G88Q, G88S, T89C, T89G, T89H, T89K, T89L, T89M, T89N, T89P, T89W, T89Y, G90A, G90C, G90D, G90L, G90S, G90T, G90V, W91E, W91L, W91P, W91Q, W91R, W91S, W91Y, Y92F, Y92H, Y92I, Y92M, Y92N, Y92S, Y92T, Y92V, Y92W, R93A, R93D, R93F, R93H, R93I, R93L, R93N, R93T, R93V, R93Y, K94A, K94C, K94G, K94R, K94S, K94T, K94V, S95A, S95C, S95D, S95E, S95G, S95I, S95L, S95Q, S95R, F96A, F96C, F96I, F96K, F96L, F96M, F96P, F96S, F96V, F96W, T97F, T97S, T97V, I98C, I98H, I98S, I98W, R100T, D101A, D101P, D101V, L102A, L102G, L102M, L102P, L102S, G104C, K105D, K105Q, K105R, K105W, K105Y, R106K, R106P, R106V, R106W, I107A, I107F, I107G, I107Q, I107S, A108E, A108S, A108V, I109M, I109T, N110A, N110F, N110S, N110T, N110V, N110W, F111A, F111C, F111L, F111Q, F111V, D112A, D112F, D112G, D112T, G113A, G113S, V114F, V114G, V114M, V114R, Y115E, M116A, M116C, M116D, M116W, M116Y, N117K, N117R, N117T, N117W, A118K, A118P, A118Y, T119A, T119G, T119L, V120A, V120K, W121C, W121D, W121R, W121T, W121V, W121Y, F122A, F122M, F122S, F122Y, N123P, G124E, G124M, G124Q, G124R, V125D, V125E, V125I, K126E, K126V, G128A, G128D, T129E, T129V, H130A, H130C, H130Q, H130S, H130T, P131K, P131L, P131S, Y132C, Y132E, Y132S, G133E, S135E, S135P, S135V, P136R, P136Y, F137A, F137C, F137D, F137G, F137L, F137P, S138A, S138D, S138G, S138H, S138L, S138M, S138R, S138V, F139A, F139E, F139Q, F139W, D140G, D140L, D140V, L141G, L141T, T142E, T142S, T142V, K146A, G148H, G148K, G148T, E150A, E150C, E150G, E150L, E150N, E150R, N151L, I153A, I153Y, V154E, V154I, V154K, V154L, V154M, V154S, V155F, V157A, V157G, V157L, V157P, V157Q, V157S, E158G, E158H, E158K, E158Q, E158V, N159D, N159H, N159S, N159T, R160G, L161E, L161K, L161M, L161S, L161W, P162F, P162G, P162N, P162T, P162W, S164A, R165H, W166S, Y167A, Y167C, S168C, G169A, G169C, G169D, G169S, S170L, S170Q, G171C, G171F, G171T, I172G, I172K, I172P, I172Q, Y173A, Y173H, Y173M, Y173P, Y173S, Y173W, R174E, R174K, D175E, D175Y, V176E, V176K, V176T, T177A, T177C, T177E, T177K, T177L, L178I, L178Q, L178W, T179A, T179C, T179D, T179H, T179I, T179K, T179L, T179N, T179P, T179S, V180A, V180C, V180D, V180E, V180G, V180M, T181A, T181D, T181F, T181K, T181R, D182F, D182L, D182S, G183W, V184F, V184H, V184P, V184Q, V184R, V184S, V184W, H185G, H185L, H185R, V186A, V186E, V186G, V186N, G187A, G187D, G187H, N188E, N188R, N188S, N188V, N188W, N189A, N189E, G190C, G190F, G190H, G190Q, G190V, V191C, V191T, V191W, V191Y, I193N, I193Q, I193T, I193V, K194A, K194I, K194L, K194R, T195A, T195E, T195M, T195S, T195W, P196A, P196H, P196I, P196M, P196S, P196W, S197A, S197C, S197E, S197K, S197L, S197P, L198E, L198F, L198H, L198I, L198K, L198R, L198V, L198W, A199E, A199F, A199K, A199P, A199R, A199T, Q201C, Q201E, Q201I, Q201K, Q201M, Q201V, N202A, N202D, N202F, N202G, N202K, N202L, N202M, N202Q, N202R, N202S, N202T, N202W, G203C, G203K, G203M, G203Q, G203R, G203S, G203V, G203W, G203Y, G204A, G204C, G204D, G204K, G204R, G204S, G204Y, V206A, V206C, V206D, V206F, V206G, V206I, V206K, V206Q, V206R, V206S, V206T, T207A, T207C, T207G, T207I, T207K, T207L, T207M, T207N, T207Q, T207R, T207W, M208A, M208S, M208T, N209C, N209D, N209G, N209K, N209L, N209Q, N209R, N209V, L210A, L210C, L210F, L210G, L210H, L210I, L210Q, L210R, L210S, L210T, L210V, T211A, T211D, T211E, T211F, T211K, T211N, T211Q, T211R, T211S, T212A, T212C, T212E, T212F, T212G, T212H, T212K, T212L, T212M, T212S, T212W, K213A, K213C, K213D, K213F, K213I, K213L, K213M, K213N, K213Q, K213R, K213S, K213T, K213V, K213Y, V214A, V214C, V214T, V214W, A215D, A215E, A215F, A215I, A215K, A215L, A215Q, A215R, A215S, A215V, N216D, N216T, N216V, D217F, D217G, D217L, D217M, D217T, D217V, T218D, T218G, T218H, A220C, A220G, A220I, A220L, A220M, A220T, A220V, A220W, A221C, A221D, A221E, A221L, A221N, A221R, A221V, A221Y, A222D, A222I, A222L, A222P, A222R, A222W, A222Y, N223A, N223E, N223F, N223G, N223K, N223L, N223M, N223R, N223S, N223T, N223V, N223W, I224G, I224Q, T225A, T225G, T225L, L226C, L226M, L226Q, K227T, Q228N, Q228R, T229A, T229C, T229D, T229G, T229H, T229M, T229N, T229Q, T229R, T229V, V230F, V230L, V230M, V230Q, V230R, V230S, F231A, F231E, F231G, F231I, F231K, F231L, F231Q, F231S, F231V, F231W, F231Y, P232G, P232H, P232L, P232M, P232R, P232S, P232T, P232V, P232W, P232Y, K233A, K233C, K233E, K233F, K233G, K233L, K233P, K233R, K233S, K233V, K233W, K233Y, G234A, G234C, G234D, G234E, G234K, G234L, G234Q, G234R, G234V, G234W, G234Y, G235C, G235F, G235H, G235l, G235K, G235M, G235Q, G235R, G235T, G235W, G235Y, K236A, K236D, K236E, K236G, K236L, K236M, K236P, K236R, K236S, K236T, K236W, K236Y, T237D, T237F, T237I, T237K, T237M, T237Q, T237R, T237S, T237V, T237Y, D238A, D238E, D238F, D238G, D238H, D238I, D238K, D238L, D238M, D238N, D238P, D238Q, D238R, A239C, A239E, A239G, A239I, A239K, A239T, A240C, A240E, A240L, A240P, A240Q, A240T, A240V, A240W, A240Y, I241T, G242K, G242L, G242M, G242P, G242T, G242Y, T243I, T243M, T243R, T243V, V244A, V244E, V244G, V244L, V244R, T245E, T245G, T245L, T245M, T245N, T245Q, T245R, T245S, T246D, T246E, T246G, T246K, T246V, A247D, A247E, A247K, A247N, A247P, A247Q, A247R, A247S, A247V, A247W, S248A, S248E, S248F, S248H, S248I, S248L, S248Q, S248T, S248Y, K249A, K249D, K249G, K249H, K249I, K249L, K249M, K249N, K249P, K249Q, K249S, K249T, K249V, K249Y, S250H, S250M, S250W, I251F, I251L, I251V, I251W, I251Y, A252C, A252E, A252F, A252H, A252I, A252P, A252R, A252S, A252W, A252Y, G254D, G254F, G254I, G254L, G254M, G254Q, G254R, G254W, A255C, A255F, A255K, A255L, A255M, A255S, A255T, A255W, A255Y, S256A, S256C, S256F, S256G, S256K, S256L, S256M, S256N, S256Q, S256R, S256V, S256W, S256Y, A257D, A257G, A257I, A257N, A257T, A257V, D258A, D258L, D258M, D258W, V259E, V259L, V259S, V259T, T260A, T260D, T260G, T260I, T260K, T260V, S261A, S261D, S261H, S261R, S261W, S261Y, T262D, T262E, T262F, T262G, T262H, T262L, T262P, T262W, I263A, I263C, I263G, I263L, I263S, I263V, A265G, A265I, A265K, A265R, A265S, A265V, S267A, S267D, S267K, S267M, S267N, S267P, S267Q, S267R, S267V, P268F, P268G, P268M, P268R, P268V, P268W, P268Y, K269G, K269R, K269V, K269Y, W271T, S272E, S272G, S272K, S272L, S272N, S272T, S272W, I273K, I273L, 1273P, I273R, I273S, I273W, K274D, K274P, K274Q, K274R, N275K, N275M, N275V, N275W, N277F, N277K, N277R, L278A, L278G, L278H, L278I, L278K, L278M, L278P, L278Q, L278R, L278S, L278V, Y279M, Y279T, Y279W, T280A, T280D, T280E, T280F, T280H, T280M, T280Q, V281A, V281I, V281L, V281Q, R282E, R282F, R282H, R282I, R282K, R282N, R282S, R282T, R282V, R282W, T283M, T283R, T283V, E284A, E284D, E284F, E284H, E284L, E284M, E284N, E284Q, E284R, E284Y, V285H, V285I, V285T, L286A, L286C, L286D, L286F, L286N, L286R, L286T, L286W, L286Y, N287I, N287L, G288F, G288L, G288S, V291C, V291D, V291F, V291G, V291H, V291L, V291P, V291S, V291T, V291Y, L292A, L292D, L292E, L292H, L292Q, L292S, L292V, D293C, D293E, D293F, D293I, D293S, D293V, D293W, T294C, T294G, T294M, T294Q, T294S, Y295F, Y295G, Y295I, Y295L, Y295R, Y295S, Y295W, D296F, D296H, D296K, D296L, D296N, D296R, D296V, T297G, T297I, T297Q, T297R, T297S, E298G, E298I, E298L, E298M, E298R, E298T, Y299F, Y299S, F301C, F301L, R302E, R302I, R302K, R302M, R302N, R302T, W303A, W303C, W303D, W303F, W303S, W303T, T304D, T304E, T304I, T304K, T304P, T304S, T304W, G305E, G305F, G305L, G305M, G305N, G305P, G305T, G305W, F306D, F306K, F306L, F306S, D307A, D307E, D307F, D307G, D307L, D307M, D307N, D307Q, D307S, D307V, D307W, A308C, A308E, A308G, A308I, A308M, A308Y, S310A, S310C, S310G, S310H, S310I, S310L, S310M, S310N, S310R, S310V, G311E, G311F, G311L, G311Q, G311R, G311V, F312H, F312L, F312R, F312V, L314A, L314C, L314T, L314V, L314Y, N315S, G316A, G316C, G316N, G316R, G316S, G316W, G316Y, E317D, E317H, E317I, E317K, E317V, E317W, E317Y, K318C, K318E, K318F, K318G, K318H, K318L, K318M, K318N, K318P, K318Q, K318V, K320C, K320E, K320F, K320I, K320L, K320R, K320T, K320V, K320W, L321C, L321G, L321H, L321M, L321V, L321Y, K322C, K322F, K322I, K322N, K322P, K322Q, K322R, K322S, V324E, V324G, V324T, S325D, S325G, S325T, M326E, M326G, M326S, M326T, M326V, M326Y, H328C, H328D, H328F, H328G, H3281, H328L, H328M, H328R, H328T, D329S, D329T, G331V, A335C, A335G, A335L, A337D, A337E, A337G, A337L, A337Q, N338P, N338R, N338V, R340A, R340C, R340L, A341M, A341S, I342A, I342G, I342K, I342P, E343A, E343H, E343N, E343R, E343S, E343T, E343Y, R344G, R344L, R344Y, Q345F, Q345G, Q345K, Q345N, Q345S, V346A, V346C, V346F, V346I, V346L, V346S, E347A, E347C, E347D, E347F, E347I, E347R, E347S, I348A, I348D, I348G, I348M, I348Q, I348R, L349A, L349E, L349Q, L349S, L349V, Q350G, Q350N, Q350W, K351N, K351R, K351V, K351W, M352G, M352L, M352T, M352W, G353K, V354E, V354G, V354S, V354W, N355H, N355M, N355R, N355W, I357T, T359E, T359L, T360V, N362G, N362S, N362T, P363A, P363D, P363I, P363L, P363M, P363S, P363V, A364C, A364E, A364F, A364G, A364I, A364M, A364P, A364V, A364W, A365C, A365E, A365I, A365P, A365V, A365W, K366A, K366D, K366E, K366I, K366L, K366M, K366P, K366S, K366V, A367C, A367I, A367N, A367Q, L368A, L368E, L368Q, L368S, L368V, L368Y, I369A, I369D, I369E, I369G, I369K, I369M, I369R, I369V, I369W, D370C, D370L, D370Q, D370R, D370S, D370T, V371D, V371F, V371G, V371I, V371L, V371Q, V371S, C372P, N373G, N373L, N373R, E374K, E374L, E374R, K375D, K375I, K375N, K375Q, K375S, G376A, G376S, V377A, V377M, V377T, L378I, L378P, L378W, L378Y, V379A, V379C, V379M, V379N, V380M, V380P, V380S, E381A, E381C, E381G, E381L, E381Q, E381T, V383A, V383K, V383L, M386G, M386N, M386Q, M386S, M386V, W387H, W387L, N388A, N388E, N388L, N388R, R389A, R389C, R389E, R389K, R389M, R389N, R389Q, R389S, R389T, R389V, S390C, S390D, S390G, S390H, S390N, S390P, S390Q, S390T, S390V, K391E, N392D, G393A, G393E, G393N, G393R, G393S, G393V, N394L, T395A, T395C, T395F, T395H, T395I, T395M, T395N, T395Q, T395S, T395W, E396K, E396L, E396M, E396V, E396W, Y398M, Y398N, G399S, K400A, K400C, K400D, K400E, K400M, K400N, K400P, K400Q, K400S, K400T, K400V, W401F, W401H, W401K, W401L, W401M, W401R, F402T, F402W, F402Y, G403A, G403D, G403H, G403K, G403P, G403Q, G403S, G403T, G403V, G403Y, Q404F, Q404H, Q404L, Q404M, Q404P, Q404R, Q404S, Q404V, I406C, I406D, I406N, A407C, A407G, A407M, A407Q, A407S, A407T, A407W, G408D, G408I, G408M, G408N, G408W, D409N, D409W, N410C, N410R, N410Y, A411E, A411N, A411R, A411S, A411V, V412M, V412S, L413D, L413E, L413F, L413I, L413P, L413T, G414A, G414C, G414M, G414N, G414R, G414T, G414W, G415A, G415Q, G415R, D416I, D416M, D416R, D416T, D416Y, K417C, K417F, K417G, K417R, K417T, D418L, D418P, D418R, D418Y, E419M, E419R, E419W, W421L, W421Q, W421S, A422P, A422T, A422V, K423D, K423L, K423M, K423R, F424C, F424L, F424N, F424T, D425E, L426C, L426M, L426Q, T427D, T427F, T427G, T427K, T427M, T427P, T427Q, T427R, T427S, T427W, S428F, S428K, S428W, T429D, T429P, I430C, I430D, I430E, I430L, I430M, I430Q, I430S, I430T, I430W, N431D, N431E, N431G, N431L, N431M, N431R, N431V, N431Y, R432A, R432E, R432F, R432G, R432N, R432Q, R432V, R432Y, D433C, D433G, D433H, D433I, D433P, D433Q, D433W, R434L, R434M, R434N, R434P, R434S, R434T, R434V, N435E, N435F, N435H, N435K, N435L, N435M, N435R, N435V, N435W, A436C, A436D, A436E, A436G, A436I, A436L, A436M, A436Q, A436S, P437A, P437D, P437K, P437L, P437Q, P437R, P437S, P437V, P437W, S438G, V439C, V439E, V439G, V439I, V439K, V439Q, V439T, V439Y, I440C, I440D, I440F, I440K, I440P, I440R, I440S, I440T, I440V, I440W, M441A, M441E, M441G, M441Q, M441R, M441T, M441V, W442E, W442G, W442M, W442P, W442Q, W442R, S443C, S443D, S443G, S443M, S443Q, S443Y, L444C, L444D, L444E, L444F, L444G, L444H, L444K, L444Q, L444V, L444W, G445A, G445C, G445V, N446D, N446T, M448A, M448C, M448D, M448E, M448I, M448L, M448P, M448Q, M448S, M448V, M448W, M449D, M449E, M449F, M449T, M449V, M449W, M449Y, E450F, E450S, E450T, E450V, E450W, G451C, G451F, G451L, G451P, G451V, G451W, I452G, I452K, I452L, I452M, I452Q, I452S, I452V, S453C, S453F, S453G, S453H, S453L, S453M, S453N, S453P, S453Q, S453R, S453V, G454L, G454W, S455A, S455E, S455K, S455M, S455P, S455R, S455V, S455W, V456A, V456D, V456E, V456F, V456K, V456L, V456W, S457E, S457H, S457K, S457L, S457M, S457P, S457Q, S457T, S457V, F459A, F459C, F459E, F459G, F459N, F459R, F459S, F459T, F459W, A461D, A461G, A461M, A461N, A461Q, A461S, A461V, A461Y, T462C, T462E, T462F, T462L, T462M, T462S, S463G, S463K, S463Q, S463R, S463T, S463V, A464E, A464H, A464L, A464M, A464P, A464V, A464W, K465C, K465F, K465G, K465L, K465Q, K465R, K465V, K465W, K465Y, L466A, L466C, L466D, L466E, L466F, L466G, L466M, L466P, L466Q, L466S, L466V, L466Y, V467A, V467C, V467D, V467E, V467G, V467T, V467W, W469A, W469C, W469D, W469G, W469L, W469M, W469R, W469V, W469Y, T470E, T470L, T470M, T470Q, K471F, K471G, K471Q, K471W, K471Y, A472G, A472Y, A473E, A473M, A473P, D474A, D474C, D474E, D474K, D474M, D474R, D474W, S475E, S475F, S475Q, S475T, S475V, T476C, T476L, T476S, R477A, R477C, R477G, R477L, P478A, P478D, P478G, P478L, P478V, M479G, M479I, M479R, M479W, T480C, T480G, T480Q, K485E, K485R, K487A, K487C, K487F, K487G, K487N, K487S, K487W, A488C, A488G, A488H, A488L, A488N, A488S, A488V, N491A, N491E, N491W, E492A, E492W, S493E, S493G, S493H, S493L, S493M, S493Q, S493V, N494A, N494I, N494M, N494R, N494V, T495K, T495R, T495V, T495W, M496A, M496F, M496T, G497D, D498A, D498C, D498M, D498S, N499K, N499R, N499T, N499Y, L500A, L500E, L500N, L500V, T501C, T501G, T501M, A502L, A502Q, N503A, N503E, N503M, N503S, G504H, G504K, G504P, G505A, G505D, G505E, G505H, G505L, G505N, G505R, G505S, G505V, V506C, V506D, V506E, V506G, V506I, V506L, V506P, V506R, V506S, V506T, V506W, V507A, V507F, V507G, V507L, V507N, V507P, V507R, V507S, V507T, G508C, G508E, T509A, T509D, T509E, T509I, T509K, T509M, T509Q, T509S, T509V, T509Y, N510A, N510F, N510I, N510Q, Y511A, Y511K, S512C, S512E, S512F, S512G, S512I, S512M, S512Q, S512T, S512V, S512Y, G514F, G514L, G514N, G514P, G514R, N516C, N516E, N516G, N516I, N516M, N516Q, N516S, N516T, N516V, Y517G, Y517I, Y517N, D518Q, D518Y, K519C, K519E, K519F, K519G, K519I, K519L, K519M, K519N, K519Q, K519S, K519T, I520A, I520C, I520G, I520H, I520M, I520N, I520Q, I520S, I520T, I520W, I520Y, R521A, R521C, R521K, R521N, R521Q, R521V, H524R, H524V, P525D, P525G, P525R, P525T, P525V, S526G, S526I, S526W, W527A, W527E, W527G, W527H, W527N, W527R, W527S, W527V, A528C, A528E, A528G, A528I, A528L, I529F, I529G, I529L, I529Y, Y530A, Y530M, G531E, G531S, G531T, T534A, T534I, T534Q, A535E, A535G, A535I, A535M, A537D, A537M, A537P, A537S, I538H, I538M, N539G, N539W, S540E, S540G, S540M, G542E, G542Q, G542S, G542T, I543V, I543W, N545G, N545I, N545Q, N545S, R546A, R546C, R546L, R546P, R546S, T547A, T547D, T547H, T547K, T547N, T547S, T548D, T548E, T548F, T548K, T548L, T548P, T548W, G549D, G549F, G549P, G549W, G550Q, G550R, G550S, A551D, A551I, A551Q, S553C, S553F, S553N, S553P, S553R, S553T, S553V, S554F, S554N, S554R, S554T, S554V, S554W, D555E, D555P, D555S, K556A, K556C, K556R, K556W, Q557F, Q557R, Q557S, L558E, L558H, L558I, L558P, T559A, T559G, T559I, T559Q, T559V, T559Y, S560P, S560V, Y561R, N563R, N563S, S564A, S564C, S564F, S564W, A565C, A565D, A565E, A565F, A565G, A565I, A565L, A565M, A565R, A565S, A565T, A565V, A565W, G567N, G567Q, G567V, A570G, A570K, A570L, A570M, A570S, A570T, A570W, V571W, A572S, A572W, S573G, S573K, S573Y, S574K, S574Q, S574V, S574W, A575D, A575M, A575V, W576A, W576F, W576V, W576Y, Y577G, Y577I, Y577L, Y577R, D578E, D578M, D578N, D578T, V579E, V579G, V579L, V579T, V580A, V580D, V580E, V580K, V580L, V580S, Q581F, Q581G, Q581P, Q581R, Q581S, Q581T, Q581Y, R582A, R582D, R582G, R582I, R582L, R582Y, D583V, D583W, F584E, F584I, F584W, V585I, V585M, V585Q, A586C, A586D, A586H, A586K, G587A, G587C, T588C, T588D, T588G, T588I, T588L, T588M, T588P, Y589A, Y589I, Y589Q, Y589V, Y589W, V590A, V590H, V590I, W591F, T592C, T592L, T592Q, T592S, G593C, G593I, F594C, F594I, F594L, F594M, D595E, D595Q, D595S, L597C, L597D, L597E, L597T, G598N, P600A, P600E, P600G, P600S, N604E, N604S, G605R, T606S, S608E, S608I, S608M, S608N, S608Q, S608T, S608V, G609A, G609H, G609L, G609N, G609R, G609S, A610D, A610F, A610M, A610T, V611K, G612N, G612T, G612V, S613A, P631A, P631D, P631G, P631H, P631S, P631V, P631Y, D633G, T634A, T634E, T634F, T634S, T634V, Y635R, Y636K, F637C, F637G, F637I, F637S, F637T, F637V, Y638A, Y638W, Q639D, Q639N, Q639R, S640C, S640D, Q641T, W642I, N643R, D644C, D644G, D644Y, D645S, D645V, V646C, V646L, V646N, V646R, V646S, H647G, H647V, T648C, L649V, H650E, H650F, H650R, I651F, I651T, I651V, L652C, L652D, L652V, L652W, P653Q, A654D, A654K, A654M, A654R, W655F, W655R, N656A, N656K, N656V, E657K, E657R, E657V, V659D, V659N, A661E, A661G, A661H, A661K, A661L, A661M, A661Q, A661W, K662H, K662S, K662V, K662W, K662Y, P669A, P669E, P669F, P669L, P669R, P669T, P669W, V670C, V672L, Y673E, Y673G, Y673I, Y673R, Y673S, Y673W, T674C, T674D, T674G, T674H, T674M, T674Q, D675A, D675E, D675P, D675Q, D675S, D675V, D675Y, A676C, A676E, A676G, A676K, A676L, A676P, A676S, A676W, A677E, A677G, A677L, A677R, A677T, A677V, A677Y, K678A, K678C, K678T, K678V, V679G, V679Q, V679S, V679T, V679Y, K680A, K680E, K680G, K680H, K680I, K680L, K680N, K680Q, K680S, K680V, K680W, L681E, L681F, L681G, L681M, L681S, L681T, Y682D, Y682E, Y682I, Y682M, Y682S, Y682V, Y682W, F683H, F683L, F683M, F683Q, F683R, F683W, T684A, T684D, T684G, T684L, T684R, T684S, T684V, P685A, P685E, P685I, P685L, P685W, K686A, K686E, K686I, K686M, K686T, K686V, G687A, G687K, G687N, G687P, G687Q, G687R, S688C, S688D, S688E, S688G, S688K, S688L, S688P, S688T, T689A, T689D, T689E, T689G, T689L, T689P, T689S, T689W, T689Y, E690D, E690M, E690P, E690T, E690V, L693A, L693M, L693P, I694L, I694W, G695C, G695K, G695L, G695R, G695W, E696A, E696L, E696R, K697A, K697E, K697G, K697R, K697V, K697W, S698C, S698D, S698E, S698I, S698L, S698M, S698P, S698Q, S698R, S698T, T700A, T700C, T700D, T700E, T700G, T700K, T700Y, K701A, K701D, K701E, K701G, K701H, K701L, K701M, K701P, K701S, K701W, T703E, T703I, T703K, T703W, T704M, T704R, T704Y, A705C, A705D, A705E, A705K, A705N, A705P, A705R, A705V, A705W, A706C, A706G, A706T, A706W, A706Y, Y708C, Y708F, Y708G, Y708K, Y708L, Y708P, Y708T, T709M, Y710C, Y710D, Y710E, Y710G, Y710M, Y710N, Y710T, Y710V, Y710W, Q711A, Q711D, Q711G, Q711L, Q711M, Q711T, Q711Y, V712G, V712M, V712P, V712Q, V712R, Y713A, Y713E, Y713G, Y713L, Y713Q, E714D, E714H, E714I, E714K, E714M, E714N, E714R, E714S, E714V, E714Y, G715K, K718A, K718L, K718Q, K718T, K718W, K718Y, S720A, S720M, S720R, S720Y, T721D, T721H, T721K, T721L, T721N, T721P, T721Q, T721S, T721V, T721W, T721Y, H723G, H723P, K724A, K724G, K724R, N725A, N725D, N725I, M726A, M726D, M726E, M726K, M726Q, M726V, M726W, Y727L, Y727T, L728C, L728K, L728Q, L728S, L728T, L728W, T729A, T729E, T729M, W730A, N731A, N731E, N731Q, N731S, N731Y, V732G, V732P, V732R, V732W, P733A, P733R, P733W, W734D, W734G, W734R, W734V, A735Q, A735R, A735S, A735T, A735W, E736S, G737F, G737K, G737N, G737Y, T738G, T738L, T738S, I739E, I739K, I739M, I739W, I739Y, S740D, S740E, S740F, S740H, S740L, A741I, A741P, A741S, A741V, E742D, E742M, E742Q, E742V, A743C, A743E, A743H, A743I, A743L, Y744A, Y744E, Y744I, Y744K, Y744L, Y744R, D745C, D745F, D745N, D745R, E746A, E746C, E746K, E746T, N747E, N747F, N747G, N747P, N747R, N748S, L750G, L750M, L750P, L750Q, L750S, I751C, I751H, I751Q, I751S, P752A, P752C, P752H, P752L, P752S, P752V, P752Y, E753Q, S755C, S755I, S755T, T756E, T756N, T756P, T756Q, T756S, T756W, E757A, G758A, G758V, N759D, N759R, N759S, N759V, A760G, A760N, A760P, A760Q, S761A, S761K, S761Q, V762D, V762G, V762K, V762W, T765A, T765P, T765R, T765W, G766M, G766S, A768H, A769F, A769G, A769I, A769N, A769V, A769Y, K770H, L771A, L771I, K772A, K772C, K772P, K772V, K772W, A773C, A773G, A773H, A773M, A773R, A773S, A773V, D774A, D774R, A775L, A775V, A775W, D776I, D776L, D776R, D776S, D776V, R777D, R777E, R777G, R777H, R777P, R777S, R777T, K778A, K778C, K778F, K778G, K778L, K778N, K778R, T779C, T779I, I780V, T781A, T781C, T781E, T781F, T781G, T781M, T781P, T781R, T781Y, A782C, A782E, A782K, A782N, A782P, A782Q, A782Y, D783A, D783C, D783E, D783R, G784A, G784F, G784L, G784S, G784T, K785C, K785I, K785S, K785V, K785W, K785Y, D786S, D786V, L787D, L787K, L787P, L787T, L787Y, S788A, S788G, S788I, Y789C, Y789D, Y789I, Y789V, I790A, I790C, I790F, I790R, I790V, E791A, E791D, E791F, E791M, E791S, E791T, E791V, E791W, V792C, V792G, V792L, V792S, V792Y, D793C, D793F, D793H, D793K, D793N, D793Y, V794C, V794D, V794L, V794Q, V794T, V794W, T795P, T795Y, D796M, D796Q, D796S, D796T, N798G, N798I, N798P, N798Q, G799D, G799K, G799L, G799M, G799Q, G799Y, H800A, H800F, H800G, H800L, H800S, H800V, I801C, I801E, I801W, V802E, V802I, V802P, V802S, V802Y, P803A, P803F, P803G, P803K, P803S, P803Y, D804E, D804G, D804K, D804N, D804S, A805C, A805F, A805G, A805I, A805N, A805P, A806F, A806I, A806Q, N807F, N807Q, N807V, N807W, R808C, R808F, R808G, R808I, R808N, R808P, R808Q, V809A, V809C, V809L, V809M, V809P, T810L, T810P, T810Q, T810R, T810Y, F811L, F811Y, D812E, D812F, D812I, D812Q, V813F, V813H, V813T, V813W, V813Y, K814G, K814H, K814I, K814L, K814P, G815A, G815F, G815M, G815P, G815V, A816C, A816D, A816F, A816I, A816N, A816V, A816W, G817C, G817H, G817I, G817N, G817S, K818D, K818F, K818L, K818Q, K818R, K818S, K818V, K818W, K818Y, L819F, L819W, V820C, V820F, V820I, V820K, V820R, V820W, G821A, G821C, G821E, G821F, G821I, G821K, G821M, G821N, G821V, G821Y, V822A, V822D, V822E, V822T, D823E, N824A, N824C, N824G, N824Q, G825A, S826A, S826F, S826G, S826I, S826L, S826R, S826W, S827C, S827Q, P828C, P828G, P828I, P828L, P828Y, D829C, D829I, D829S, D829T, D829V, H830E, H830G, H830M, H830P, H830Q, H830R, H830V, D831A, D831F, D831G, D831I, D831M, D831P, D831R, D831V, S832E, S832F, S832G, S832L, S832M, S832P, S832R, S832V, S832W, Y833C, Y833D, Y833E, Y833I, Y833K, Y833N, Y833P, Y833V, Q834F, Q834G, Q834M, A835D, A835E, A835F, A835H, A835K, A835W, D836C, D836E, D836H, D836Q, D836R, D836S, D836T, D836V, D836W, D836Y, N837D, N837F, N837G, N837H, N837L, N837P, N837T, R838D, R838F, R838G, R838K, R838M, R838N, R838S, R838W, A840G, A840I, A840P, A840V, F841C, F841D, F841I, F841K, F841W, S842A, S842L, S842M, G843A, G843C, K844A, K844G, K844L, K844W, K844Y, V845N, V845W, L846G, L846I, L846M, L846S, A847L, A847T, I848M, V849A, V849L, V849S, V849T, Q850C, Q850G, Q850I, Q850L, Q850T, Q850V, Q850Y, S851C, S851D, S851E, S851L, S851T, T852D, T852G, T852L, K853N, K853P, K853Q, K853V, E854C, E854I, E854M, E854R, A855K, A855V, A855Y, E857P, E857V, I858D, I858E, I858F, I858G, I858K, I858M, I858P, I858Q, I858Y, T859V, V860T, V860Y, T861F, T861I, T861Q, T861V, T861W, A862C, A862P, A862V, K863F, K863I, K863L, K863N, K863W, A864E, A864H, A864K, A864L, D865A, D865G, G866H, G866R, S870E, S870M, S870R, S870T, T871P, V872C, V872G, K873G, K873Y, I874G, A875R, T877A, V879P, V879S, P880S, G881W, T882A, T882M, T882R, S883L, T884A, E885V, K886E, K886L, K886V, K886W, T887A, T887D, T887F, T887G, T887N, T887R, T887V, V888A, V888D, V888G, R889G, Y893E, Y893G, S894D, S894G, R895M, N896M, Y897V, Y898T, V899G, K900E, K900G, T901G, T901Q, T901R, T901V, T901Y, G902A, G902D, G902F, G902L, G902P, G902Q, G902R, G902S, G902W, N903D, N903I, K904D, K904E, K904M, K904N, K904R, K904S, K904V, K904W, P905A, P905C, P905R, P905V, P905W, P905Y, L907F, L907S, L907Y, P908C, P908D, P908G, P908I, P908L, P908M, P908T, S909E, S909F, S909G, S909W, S909Y, D910C, D910I, D910S, D910W, V911A, V911S, E912A, E912K, E912L, E912T, E912V, V913G, V913Q, V913R, V913W, R914A, R914E, R914F, R914I, R914K, R914V, R914W, R914Y, Y915A, Y915C, Y915G, Y915I, Y915M, Y915Q, Y915V, S916G, D917C, D917E, D917F, D917L, D917M, D917Q, D917S, G918E, G918H, G918T, G918V, G918W, T919D, T919K, T919Q, T919W, T919Y, S920C, S920E, S920M, S920P, S920R, S920V, S920W, D921C, D921P, D921Q, D921V, R922A, R922G, R922M, R922V, R922W, Q923A, Q923C, Q923E, Q923L, Q923M, Q923V, Q923W, N924A, N924L, N924P, N924Q, N924S, N924W, V925A, V925C, V925E, V925G, V925K, V925N, V925S, V925W, T926G, T926P, T926R, T926S, T926V, T926W, W927C, W927G, W927P, D928A, D928E, D928H, D928L, D928Q, A929C, A929P, A929V, V930A, V930E, V930I, V930K, V930M, V930T, S931G, S931P, S931R, D932F, D932R, D932S, D932T, D932V, D933I, D933R, D933S, Q934S, Q934V, I935A, I935C, I935D, I935E, I935L, I935P, I935V, I935W, A936I, A936L, A936Q, A936R, A936Y, K937G, K937I, K937M, K937P, K937Q, K937R, K937V, A938C, A938H, A938N, A938T, A938V, A938W, G939D, G939K, S940C, S940E, S940M, S940R, S940T, S940V, S940W, F941C, F941M, F941W, S942A, S942E, S942K, S942L, S942P, S942T, S942V, V943A, V943G, V943H, V943Q, V943R, A944D, A944G, A944H, A944P, A944R, A944V, G945E, G945P, G945T, T946A, T946E, T946G, T946L, T946P, T946V, T946W, V947G, V947H, V947L, V947M, V947P, V947R, V947T, A948C, A948I, A948R, A948W, G949A, G949F, G949V, Q950D, Q950G, Q950K, Q950M, Q950W, K951D, K951G, K951P, K951Q, K951S, K951W, K951Y, I952H, I952Q, S953F, S953M, S953N, S953R, S953W, V954D, V954L, V954Q, V954S, V954T, R955A, R955C, R955E, R955K, R955Q, R955W, V956A, V956D, V956G, V956H, V956I, V956M, V956Q, V956W, T957D, T957S, T957W, M958D, M958I, M958K, I959A, I959L, I959S, I959V, I959Y, D960G, D960H, D960L, D960P, D960S, D960W, E961D, E961F, E961K, E961P, E961S, E961T, I962A, I962C, I962D, I962G, I962K, I962N, I962Q, I962T, G963C, G963E, G963L, G963P, A964C, A964E, A964H, L965C, L965E, L965G, L965K, L965M, L965P, L965Q, L965S, L965V, L965Y, L966A, L966G, L966H, L966K, L966N, L966P, L966Q, L966S, L966T, L966V, N967C, N967D, N967I, N967L, N967M, N967P, N967S, N967T, N967V, Y968G, Y968L, Y968Q, Y968V, S969C, S969D, S969G, S969H, S969I, S969L, S969M, S969P, S969Q, S969Y, A970I, A970L, P973C, P973D, P973K, P973N, P973Q, P973R, P973V, P973W, P973Y, V974C, V974E, V974G, V974N, V974T, V974Y, G975D, G975F, G975K, G975L, G975Q, G975V, T976D, T976F, T976G, T976K, T976L, T976P, T976S, T976Y, P977A, P977C, P977K, P977R, P977T, P977Y, A978F, A978G, A978M, A978N, A978P, A978R, A978S, A978Y, V979G, V979N, V979R, V979Y, L980A, L980F, L980H, L980I, L980K, L980N, L980Q, L980T, L980Y, P981L, P981M, R984P, R984S, P985E, P985F, P985H, P985K, P985L, P985W, A986C, A986E, A986F, A986I, A986K, A986L, A986M, A986N, A986S, A986W, V987A, V987C, V987F, V987I, V987K, V987L, V987Q, V987T, L988A, L988C, L988E, L988G, L988H, L988M, L988Q, L988R, L988S, L988V, L988Y, P989A, P989C, P989D, P989G, P989H, P989I, P989M, P989N, P989Q, P989W, D990F, D990P, D990S, D990W, G991C, G991F, G991H, G991K, G991P, G991Y, T992E, T992H, T992M, T992N, T992Y, V993D, V993G, V993N, V993S, T994I, T994S, T994V, S995E, S995L, S995R, S995V, A996Q, A996R, A996V, N997A, N997C, N997E, N997K, N997L, N997S, N997V, N997W, N997Y, F998M, F998W, A999F, A999G, A999L, A999M, A999R, A999S, V1000C, V1000L, V1000M, V1000N, V1000P, V1000W, W1002A, W1002D, W1002E, W1002H, W1002N, W1002P, W1002Q, W1002S, T1003F, T1003G, T1003L, T1003N, T1003P, T1003R, T1003S, T1003W, T1003Y, K1004D, K1004E, K1004F, K1004G, K1004H, K1004M, K1004P, K1004R, K1004S, K1004V, P1005I, P1005N, P1005Q, P1005V, P1005Y, A1006C, A1006I, A1006N, A1006P, A1006S, A1006V, A1006W, A1006Y, D1007C, D1007L, D1007P, D1007V, T1008G, V1009G, V1009S, Y1010A, Y1010P, Y1010R, Y1010T, N1011A, N1011S, N1011T, N1011W, T1012E, T1012H, T1012I, T1012Q, T1012Y, A1013D, A1013K, A1013Q, A1013T, A1013V, G1014E, G1014I, G1014L, G1014M, G1014V, G1014W, G1014Y, T1015A, T1015F, T1015G, T1015V, V1016C, V1016D, V1016P, K1017E, K1017G, V1018I, V1018K, V1018L, V1018M, V1018R, V1018S, V1018W, T1021C, T1021E, T1021F, T1021G, T1021K, T1021L, T1021S, T1021V, A1022H, A1022L, A1022S, A1022Y, T1023D, T1023M, T1023Q, T1023R, V1024E, V1024G, V1024H, V1024K, V1024N, V1024R, V1024S, V1024W, G1026E, G1026H, G1026L, G1026R, G1026S, G1026V, G1026Y, K1027C, K1027N, K1027Q, K1027R, K1027V, E1028G, E1028S, E1028T, F1029I, F1029K, F1029L, F1029P, F1029V, F1029W, F1029Y, K1030D, K1030F, K1030H, K1030L, K1030M, K1030W, V1031H, V1031K, V1031Y, A1033G, A1033S, A1033V, T1034G, T1034H, T1034N, T1034W, I1035D, I1035G, I1035Q, R1036G, R1036L, R1036T, R1036Y, V1037C, V1037F, V1037P, V1037Q, Q1038A, Q1038D, Q1038K, R1039S, R1039V, S1040A, S1040M, S1040N, S1040R, S1040W, Q1041P, V1042N, T1043F, T1043G, T1043N, T1043R, I1044A, I1044L, G1045S, S1046I, S1046M, S1047D, V1048C, V1048F, V1048G, V1048I, V1048M, V1048Q, G1050L, G1050S, G1050V, N1051A, N1051E, N1051K, A1052C, A1052K, A1052M, A1052P, A1052R, L1053A, L1053W, L1055R, L1055T, Q1057A, Q1057E, Q1057P, Q1057R, N1058R, N1058S, N1058V, N1058W, I1059W, P1060G, P1060N, P1060Q, P1060S, P1060T, D1062A, D1062F, D1062G, D1062I, D1062L, D1062M, D1062P, D1062S, K1063D, K1063M, Q1064C, Q1064M, Q1064R, Q1064T, Q1064V, S1065A, S1065C, S1065E, S1065G, S1065T, S1065W, D1066A, D1066G, D1066M, D1066V, D1066W, T1067G, T1067M, L1068C, L1068E, L1068P, L1068Q, L1068Y, A1070P, A1070T, I1071M, I1071R, I1071W, K1072E, K1072G, K1072P, K1072Q, K1072S, D1073F, D1073L, D1073M, D1073P, D1073W, G1074I, G1074L, G1074R, S1075C, S1075G, S1075I, S1075L, S1075V, T1076C, T1076E, T1076H, T1076Q, T1076S, T1077K, T1077L, T1077R, V1078D, V1078E, V1078L, V1078W, D1079G, D1079L, N1081D, N1081E, N1081G, T1082A, T1082C, T1082E, T1082F, T1082G, T1082K, T1082N, T1082S, G1083E, G1083F, G1083L, G1083P, G1083S, G1084C, G1084M, G1084V, G1084W, G1084Y, G1085A, G1085P, G1085R, G1085S, A1086H, A1086K, A1086Q, A1086R, A1086T, N1087A, N1087E, N1087I, N1087R, N1087V, N1087W, P1088D, P1088E, P1088G, P1088R, P1088W, S1089C, S1089E, S1089G, S1089K, S1089Q, S1089R, S1089V, A1090F, A1090G, A1090I, A1090K, W1091A, W1091E, W1091G, W1091H, W1091T, W1091V, W1091Y, T1092A, T1092E, T1092G, T1092K, T1092Q, T1092S, T1092V, N1093A, N1093G, N1093L, N1093P, N1093Q, N1093T, N1093V, W1094D, W1094E, W1094P, W1094R, W1094T, A1095P, A1095R, A1095T, A1095W, Y1096A, Y1096D, Y1096H, Y1096L, Y1096R, S1097D, S1097E, S1097K, S1097L, S1097T, S1097W, K1098D, K1098F, K1098G, K1098Q, K1098S, A1099C, A1099D, A1099F, A1099S, A1099V, A1099W, G1100D, G1100E, G1100H, G1100M, G1100N, G1100T, H1101K, H1101L, H1101Q, H1101R, H1101V, N1102E, N1102F, N1102H, N1102K, N1102L, N1102Q, N1102R, N1102T, T1103A, T1103E, T1103H, T1103S, T1103W, A1104I, A1104K, A1104R, E1105L, E1105S, I1106T, I1106V, T1107C, T1107M, T1107R, T1107S, F1108D, F1108K, F1108L, F1108T, F1108W, E1109A, E1109D, E1109L, E1109W, A1111G, A1111S, E1113D, E1113G, E1113P, E1113V, Q1114E, Q1114I, Q1114L, Q1114R, Q1114S, Q1114T, Q1114V, Q1115A, Q1115K, Q1115L, Q1115P, Q1115T, Q1115W, L1116D, L1116G, L1116H, L1116K, L1116V, L1116W, G1117E, G1117I, G1117M, G1117R, G1117S, G1117T, G1117V, G1117W, Q1118A, Q1118M, Q1118S, Q1118T, Q1118W, I1119D, I1119E, I1119G, I1119N, I1119S, V1120N, V1120S, V1120T, M1121G, M1121K, M1121N, M1121P, M1121S, M1121V, M1121Y, Y1122A, Y1122C, Y1122I, Y1122K, Y1122R, Y1122V, Y1122W, F1123E, F1123H, F1123R, F1123T, F1124E, F1124R, F1124V, F1124W, R1125D, R1125E, R1125F, R1125K, R1125T, R1125V, R1125W, D1126H, D1126K, D1126L, D1126R, S1127F, S1127I, S1127K, S1127M, S1127Q, S1127T, S1127W, N1128A, N1128C, N1128R, N1128S, N1128T, N1128W, A1129E, A1129L, A1129N, A1129Q, A1129R, A1129V, V1130A, V1130G, V1130P, V1130R, V1130S, R1131A, R1131N, R1131Q, R1131S, R1131W, F1132E, F1132K, F1132M, F1132P, F1132Q, F1132T, P1133D, P1133G, P1133L, P1133Q, P1133R, P1133V, D1134E, D1134G, D1134L, A1135E, A1135K, A1135L, A1135M, A1135S, A1135W, A1135Y, G1136A, G1136E, G1136P, G1136Q, G1136T, K1137A, K1137C, K1137G, K1137L, K1137P, K1137Q, K1137R, K1137S, K1137T, K1137V, T1138R, T1138Y, K1139A, K1139L, K1139R, K1139T, I1140A, I1140C, I1140G, I1140L, I1140M, I1140P, I1140R, I1140T, Q1141A, Q1141C, Q1141G, Q1141K, Q1141N, Q1141P, Q1141T, Q1141W, I1142E, I1142R, I1142S, I1142W, I1142Y, S1143G, A1144C, A1144D, A1144E, A1144N, A1144P, A1144S, A1144T, A1144V, A1144W, D1145E, D1145R, D1145T, G1146A, G1146C, G1146D, G1146K, G1146L, G1146R, G1146V, K1147A, K1147G, K1147T, K1147V, N1148H, N1148I, N1148K, N1148P, N1148Q, N1148R, N1148S, N1148T, N1148W, W1149C, W1149G, W1149I, W1149K, W1149N, W1149Q, W1149S, W1149T, W1149V, W1149Y, T1150G, T1150K, T1150P, D1151C, D1151G, D1151R, D1151T, D1151W, L1152A, L1152C, L1152E, L1152Q, L1152W, A1153E, A1153G, A1153K, A1153L, A1154C, A1154D, A1154E, A1154G, A1154R, A1154S, T1155E, T1155L, T1155Q, T1155R, T1157V, T1157W, I1158R, I1158S, I1158W, A1159C, A1159E, A1159I, A1159P, A1159R, A1159V, Q1161A, Q1161P, Q1161S, E1162A, E1162C, E1162D, E1162F, E1162I, E1162N, E1162Q, E1162T, E1162W, E1162Y, R1166D, R1166K, R1166Q, V1167A, V1167C, V1167L, V1167P, V1167R, K1168L, K1168Q, K1168R, K1168W, P1169M, P1169R, P1169S, Y1170E, Y1170K, Y1170M, Y1170Q, Y1170R, Y1170V, T1171A, T1171G, T1171M, T1171Q, T1171R, T1171S, Y1172D, Y1172E, Y1172H, Y1172I, Y1172K, Y1172L, Y1172S, Y1172V, D1173A, D1173E, D1173F, D1173G, D1173K, D1173L, D1173P, D1173R, D1173T, D1173W, F1174D, F1174P, F1174Q, F1174R, F1174S, F1174T, F1174V, F1174W, A1175G, A1175I, A1175N, A1175Q, A1175S, A1175V, A1175Y, V1177N, V1177P, V1177S, V1177T, G1178M, G1178Q, G1178S, G1178T, A1179L, A1179P, A1179Q, A1179W, T1180A, T1180G, T1180I, T1180L, T1180M, T1180Q, T1180S, T1180Y, F1181E, F1181L, F1181V, V1182M, V1184C, V1184E, V1184K, V1184L, V1184P, V1184Q, V1184Y, T1185Q, T1185V, V1186S, N1188V, N1188W, A1189C, A1189K, A1189T, A1189V, D1190R, D1190S, D1190T, D1190Y, T1191E, T1191L, T1192H, T1192P, T1193G, P1194A, P1194E, P1194G, P1194W, S1195G, V1197A, V1198E, C1199D, C1199T, L1202A, L1202C, T1203E, T1203K, E1204G, E1204S, I1205V, K1208R, K1208V, K1208W, T1209A, T1209C, T1209E, T1209K, T1209N, T1209Q, T1209R, T1209W, A1210D, A1210E, A1210G, A1210K, A1210L, A1210Q, A1210R, A1210T, A1210W, T1211C, T1211D, T1211E, T1211G, T1211H, T1211K, T1211P, T1211Q, T1211R, T1211S, T1211V, K1213A, K1213D, K1213S, K1213T, K1213W, F1214A, F1214E, F1214K, F1214L, F1214P, F1214R, F1214S, F1214V, V1215D, V1215E, V1215K, V1215L, V1215Q, V1215S, V1215W, N1217A, N1217D, N1217E, N1217F, N1217P, N1217R, N1217S, N1217T, T1218A, T1218C, T1218E, T1218G, T1218Q, T1218S, T1218V, T1218W, S1219A, S1219E, S1219F, S1219I, S1219K, S1219R, S1219V, A1220C, A1220G, A1220L, A1220P, A1220R, A1220V, A1221D, A1221G, A1221K, A1221L, A1221R, A1221V, A1221W, L1222A, L1222C, L1222E, L1222F, L1222Q, L1222R, L1222V, L1222W, S1223C, S1223F, S1223G, S1223K, S1223L, S1223V, S1224A, S1224D, S1224G, S1224L, S1224M, S1224P, S1224R, S1224W, L1225C, L1225D, L1225E, L1225F, L1225G, L1225K, L1225P, L1225T, L1225V, L1225W, T1226A, T1226G, T1226M, T1226P, T1226R, T1226S, T1226V, T1226Y, V1227A, V1227C, V1227D, V1227E, V1227G, V1227L, V1227P, V1227Q, V1227S, N1228A, N1228D, N1228F, N1228K, N1228L, N1228T, T1230F, T1230H, T1230I, T1230K, T1230L, T1230P, T1230R, T1230S, T1230W, K1231F, K1231G, K1231L, K1231M, K1231P, K1231S, K1231W, V1232E, V1232K, V1232Q, V1232R, V1232S, V1232T, V1232W, S1233P, S1233W, D1234G, D1234K, D1234R, D1234V, S1235D, S1235E, S1235G, S1235L, S1235P, S1235R, S1235W, S1235Y, V1236A, V1236C, V1236G, V1236I, V1236P, V1236Q, V1236R, L1237D, L1237E, L1237R, L1237V, L1237W, A1238D, A1238E, A1238K, A1238L, A1238N, A1238P, A1238R, A1238S, A1238T, A1239D, A1239P, A1239R, G1240D, G1240L, G1240N, G1240Q, G1240S, G1240T, G1240W, S1241D, S1241G, S1241I, S1241L, S1241M, S1241P, Y1242C, Y1242E, Y1242K, Y1242R, Y1242S, Y1242W, N1243C, N1243L, N1243M, N1243P, N1243Q, N1243S, N1243T, N1243V, N1243W, T1244A, T1244D, T1244E, T1244G, T1244L, T1244Q, T1244S, T1244V, T1244W, A1246F, A1246M, A1246N, A1246P, A1246Q, A1246R, A1246S, A1246T, I1247A, I1247G, I1247M, I1247Q, I1247R, I1247S, I1247T, I1247V, I1247W, I1248A, I1248G, I1248K, I1248L, I1248R, I1248S, I1248Y, A1249E, A1249G, A1249H, A1249I, A1249R, A1249T, A1249V, D1250I, D1250K, D1250S, D1250T, D1250W, D1250Y, V1251I, V1251T, V1251W, K1252D, K1252G, K1252V, K1252W, A1253P, A1253V, E1254F, E1254G, E1254H, E1254L, E1254R, E1254V, G1255H, G1255M, G1255S, G1255V, E1256G, E1256M, E1256N, E1256R, E1256V, E1256W, G1257F, G1257K, G1257L, G1257Q, G1257R, G1257W, N1258C, N1258G, N1258H, N1258K, N1258S, A1259K, A1259L, A1259W, V1261I, V1261L, V1261P, V1261Q, V1261R, V1261T, T1262A, T1262F, T1262M, T1262Q, T1262R, V1263E, V1263G, V1263Q, V1263R, V1263T, V1263W, L1264A, L1264E, L1264H, L1264R, L1264S, L1264Y, P1265C, P1265K, P1265L, P1265R, P1265S, P1265V, P1265W, A1266F, A1266L, A1266P, A1266S, A1266V, H1267A, H1267E, H1267F, N1269A, N1269E, N1269K, N1269R, N1269S, N1269T, N1269W, V1270D, V1270E, V1270G, V1270I, V1270L, V1270T, V1270W, I1271A, I1271H, I1271Q, R1272E, R1272F, R1272M, R1272P, R1272V, V1273R, I1274F, I1274M, I1274R, T1275A, T1275L, T1275W, E1276R, E1276W, S1277L, S1277T, S1277W, E1278Q, E1278R, D1279G, D1279I, D1279R, D1279T, D1279V, D1279W, H1280C, H1280E, H1280G, H1280V, H1280W, V1281F, V1281I, V1281S, V1281W, T1282D, T1282L, T1282V, R1283A, R1283D, R1283E, R1283P, R1283W, K1284G, T1285A, T1285E, T1285F, T1285G, T1285M, T1285R, T1285Y, F1286A, F1286E, F1286P, F1286R, F1286S, F1286T, T1287C, T1287K, T1287L, T1287M, T1287Q, T1287R, T1287S, T1287W, I1288A, I1288D, I1288F, I1288G, I1288K, N1289A, N1289Q, N1289T, L1290A, L1290G, L1290R, L1290V, L1290W, G1291K, G1291P, G1291V, G1291W, G1291Y, T1292G, T1292L, T1292Y, E1293G, E1293K, E1293L, E1293S, E1293V, Q1294E, Q1294L, Q1294P, Q1294W, E1295K, F1296A, F1296G, F1296I, P1297F, A1298Y, D1301I, E1302G, E1302R, E1302S, R1303S, D1304A or D1304V.

3. A lactase variant having an amino acid sequence which is at least 80% identical to SEQ ID NO: 1 which comprises one or more of the following substitutions, wherein each position corresponds to a position in SEQ ID NO: 1: V1D, V1F, V1H, V1K, V1L, V1R, E2D, E2G, E2Q, E2V, D3A, D3H, D3I, D3N, D3S, D3V, D3W, A4C, A4G, A4H, A4I, A4L, A4M, A4P, T5A, T5D, T5F, T5K, T5R, T5S, T5V, R6A, R6D, R6F, R6G, R6H, R6L, R6M, R6P, R6S, R6W, S7A, S7D, S7I, S7L, S7N, S7P, S7T, S7W, S9G, S9H, S9P, S9R, S9W, T10G, T10K, T10L, T10P, T10R, T10S, T10W, T11L, T11P, Q12L, Q12R, Q12V, Q12Y, M13C, M13D, M13E, M13F, M13H, M13K, M13R, M13W, S14A, S14G, S14H, S14L, S14T, S14V, S14Y, S15C, S15F, S15I, S15K, S15L, S15P, S15R, S15T, S15V, S15W, S15Y, T16A, T16C, T16I, T16N, T16S, P17A, P17C, P17D, P17E, P17I, P17L, P17N, P17R, P17S, P17T, V19A, V19F, V19G, V19I, V19K, V19L, V19N, V19S, V19W, V20C, V20F, V20G, V20I, V20K, V20L, V20M, V20N, V20P, V20Q, V20R, V20T, V20W, Y21A, Y21C, Y21D, Y21F, Y21G, Y21H, Y21M, Y21P, Y21R, Y21T, S22A, S22E, S22F, S22G, S22L, S22M, S22N, S22R, S22T, S22W, S23A, S23C, S23D, S23L, S23M, S23R, A24F, A24G, A24L, A24R, A24T, A24W, V25D, V25E, V25F, V25G, V25H, V25K, V25L, V25M, V25Q, V25R, V25S, V25T, V25W, D26C, D26I, D26L, D26M, D26T, D26V, S27A, S27C, S27F, S27G, S27H, S27P, S27Y, K28C, K28G, K28I, K28L, K28R, K28S, K28V, K28W, Q29D, Q29F, Q29G, Q29L, Q29M, Q29R, Q29S, Q29V, Q29W, N30A, N30G, N30H, N30M, N30P, N30V, N30W, N30Y, R31E, R31G, R31I, R31M, R31V, T32M, T32Q, T32R, T32S, S33C, S33E, S33H, S33K, S33N, S33Q, S33R, S33V, D34C, D34E, D34F, D34G, D34H, D34L, D34S, D34W, D34Y, F35A, F35C, F35E, F35G, F35K, F35N, F35T, F35V, D36H, D36Q, A37N, A37Q, N38C, N38G, N38S, W39G, W39S, K40C, K40D, K40F, K40G, K40I, K40M, K40N, K40P, K40W, F41A, F41C, F41G, F41I, F41Q, F41S, F41Y, M42E, M42N, M42T, L43A, L43C, L43G, L43I, L43S, L43T, L43V, S44C, S44M, S44N, S44Y, D45A, D45L, D45P, D45V, V47K, V47R, Q48S, A49C, A49D, A49H, A49R, A49S, A49T, A49V, D51G, D51I, D51K, D51M, D51P, D51V, A53C, A53G, A53L, A53R, A53S, A53V, A53W, F54M, F54S, D55C, D55F, D55G, D55H, D55M, D55N, D55P, D55S, D55V, S57A, S57C, S57E, S57G, A58D, A58G, A58I, A58M, A58N, A58Q, A58R, A58T, W59D, W591, W59K, W59L, W59N, W59P, W59V, Q60A, Q60C, Q60E, Q60F, Q60G, Q60K, Q60L, Q60M, Q60R, Q60S, Q60V, Q60Y, Q61K, Q61P, Q61S, V62G, V62N, V62S, V62T, V62W, D63G, D63L, D63P, D63S, D63V, L64E, L64G, H66C, H66L, H66R, H66T, H66W, H66Y, D67E, Y68P, Y68V, 170A, I70H, I70K, I70P, I70R, T71C, T71E, T71G, T71H, T71K, T71L, T71N, T71P, T71Q, T71R, T71S, K73A, K73D, K73F, K73G, K73Q, K73V, Y74G, Y74K, Y74T, Y74W, S75G, S75L, S75Q, S75R, S75V, Q76G, Q76I, Q76K, Q76M, Q76P, Q76S, Q76V, Q76Y, S77C, S77D, S77E, S77F, S77G, S77H, S77I, S77K, S77L, S77M, S77R, S77T, S77V, S77W, S77Y, N78C, N78E, N78F, N78K, N78Q, N78R, N78S, N78T, E79H, E79Q, E79S, E79T, E79W, A80K, E81A, E81Q, A83E, A83T, L85A, L85C, L85D, L85F, L85M, L85N, L85S, L85V, L85W, P86E, P86G, P86H, P86N, P86Q, P86R, P86V, P86W, P86Y, G87A, G87D, G87E, G87N, G87Q, G88A, G88F, G88I, G88M, G88Q, G88S, T89C, T89G, T89H, T89K, T89L, T89M, T89N, T89P, T89W, T89Y, G90A, G90C, G90D, G90L, G90S, G90T, G90V, W91E, W91L, W91P, W91Q, W91R, W91S, W91Y, Y92F, Y92H, Y92I, Y92M, Y92N, Y92S, Y92T, Y92V, Y92W, R93A, R93D, R93F, R93H, R93I, R93L, R93N, R93T, R93V, R93Y, K94A, K94C, K94G, K94R, K94S, K94T, K94V, S95A, S95C, S95D, S95E, S95G, S95I, S95L, S95Q, S95R, F96A, F96C, F96I, F96K, F96L, F96M, F96P, F96S, F96V, F96W, T97F, T97S, T97V, I98C, I98H, I98S, I98W, R100T, D101A, D101P, D101V, L102A, L102G, L102M, L102P, L102S, G104C, K105D, K105Q, K105R, K105W, K105Y, R106K, R106P, R106V, R106W, I107A, I107F, I107G, I107Q, I107S, A108E, A108S, A108V, I109M, I109T, N110A, N110F, N110S, N110T, N110V, N110W, F111A, F111C, F111L, F111Q, F111V, D112A, D112F, D112G, D112T, G113A, G113S, V114F, V114G, V114M, V114R, Y115E, M116A, M116C, M116D, M116W, M116Y, N117K, N117R, N117T, N117W, A118K, A118P, A118Y, T119A, T119G, T119L, V120A, V120K, W121C, W121D, W121R, W121T, W121V, W121Y, F122A, F122M, F122S, F122Y, N123P, G124E, G124M, G124Q, G124R, V125D, V125E, V125I, K126E, K126V, G128A, G128D, T129E, T129V, H130A, H130C, H130Q, H130S, H130T, P131K, P131L, P131S, Y132C, Y132E, Y132S, G133E, S135E, S135P, S135V, P136R, P136Y, F137A, F137C, F137D, F137G, F137L, F137P, S138A, S138D, S138G, S138H, S138L, S138M, S138R, S138V, F139A, F139E, F139Q, F139W, D140G, D140L, D140V, L141G, L141T, T142E, T142S, T142V, K146A, G148H, G148K, G148T, G149H, G149I, G149M, G149Q, G149Y, E150A, E150C, E150G, E150L, E150N, E150R, N151L, I153A, I153Y, V154E, V154I, V154K, V154L, V154M, V154S, V155F, V157A, V157G, V157L, V157P, V157Q, V157S, E158G, E158H, E158K, E158Q, E158V, N159D, N159H, N159S, N159T, R160G, L161E, L161K, L161M, L161S, L161W, P162F, P162G, P162N, P162T, P162W, S164A, R165H, W166S, Y167A, Y167C, S168C, G169A, G169C, G169D, G169S, S170L, S170Q, G171C, G171F, G171T, I172G, I172K, I172P, I172Q, Y173A, Y173H, Y173M, Y173P, Y173S, Y173W, R174E, R174K, D175E, D175Y, V176E, V176K, V176T, T177A, T177C, T177E, T177K, T177L, L178I, L178Q, L178W, T179A, T179C, T179D, T179H, T179I, T179K, T179L, T179N, T179P, T179S, V180A, V180C, V180D, V180E, V180G, V180M, T181A, T181D, T181F, T181K, T181R, D182F, D182L, D182S, G183W, V184F, V184H, V184P, V184Q, V184R, V184S, V184W, H185G, H185L, H185R, V186A, V186E, V186G, V186N, G187A, G187D, G187H, N188E, N188R, N188S, N188V, N188W, N189A, N189E, G190C, G190F, G190H, G190Q, G190V, V191C, V191T, V191W, V191Y, I193N, I193Q, I193T, I193V, K194A, K194I, K194L, K194R, T195A, T195E, T195M, T195S, T195W, P196A, P196H, P196I, P196M, P196S, P196W, S197A, S197C, S197E, S197K, S197L, S197P, L198E, L198F, L198H, L198I, L198K, L198R, L198V, L198W, A199E, A199F, A199K, A199P, A199R, A199T, Q201C, Q201E, Q201I, Q201K, Q201M, Q201V, N202A, N202D, N202F, N202G, N202K, N202L, N202M, N202Q, N202R, N202S, N202T, N202W, G203C, G203K, G203M, G203Q, G203R, G203S, G203V, G203W, G203Y, G204A, G204C, G204D, G204K, G204R, G204S, G204Y, N205E, N205G, N205H, N205L, N205P, N205W, N205Y, V206A, V206C, V206D, V206F, V206G, V206I, V206K, V206Q, V206R, V206S, V206T, T207A, T207C, T207G, T207I, T207K, T207L, T207M, T207N, T207Q, T207R, T207W, M208A, M208S, M208T, N209C, N209D, N209G, N209K, N209L, N209Q, N209R, N209V, L210A, L210C, L210F, L210G, L210H, L210I, L210Q, L210R, L210S, L210T, L210V, T211A, T211D, T211E, T211F, T211K, T211N, T211Q, T211R, T211S, T212A, T212C, T212E, T212F, T212G, T212H, T212K, T212L, T212M, T212S, T212W, K213A, K213C, K213D, K213F, K213I, K213L, K213M, K213N, K213Q, K213R, K213S, K213T, K213V, K213Y, V214A, V214C, V214T, V214W, A215D, A215E, A215F, A215I, A215K, A215L, A215Q, A215R, A215S, A215V, N216D, N216T, N216V, D217F, D217G, D217L, D217M, D217T, D217V, T218D, T218G, T218H, K219A, K219C, K219F, K219H, K219M, A220C, A220G, A220I, A220L, A220M, A220T, A220V, A220W, A221C, A221D, A221E, A221L, A221N, A221R, A221V, A221Y, A222D, A222I, A222L, A222P, A222R, A222W, A222Y, N223A, N223E, N223F, N223G, N223K, N223L, N223M, N223R, N223S, N223T, N223V, N223W, I224G, I224Q, T225A, T225G, T225L, L226C, L226M, L226Q, K227T, Q228N, Q228R, T229A, T229C, T229D, T229G, T229H, T229M, T229N, T229Q, T229R, T229V, V230F, V230L, V230M, V230Q, V230R, V230S, F231A, F231E, F231G, F231I, F231K, F231L, F231Q, F231S, F231V, F231W, F231Y, P232G, P232H, P232L, P232M, P232R, P232S, P232T, P232V, P232W, P232Y, K233A, K233C, K233E, K233F, K233G, K233L, K233P, K233R, K233S, K233V, K233W, K233Y, G234A, G234C, G234D, G234E, G234K, G234L, G234Q, G234R, G234V, G234W, G234Y, G235C, G235F, G235H, G235l, G235K, G235M, G235Q, G235R, G235T, G235W, G235Y, K236A, K236D, K236E, K236G, K236L, K236M, K236P, K236R, K236S, K236T, K236W, K236Y, T237D, T237F, T237I, T237K, T237M, T237Q, T237R, T237S, T237V, T237Y, D238A, D238E, D238F, D238G, D238H, D238I, D238K, D238L, D238M, D238N, D238P, D238Q, D238R, A239C, A239E, A239G, A239I, A239K, A239T, A240C, A240E, A240L, A240P, A240Q, A240T, A240V, A240W, A240Y, I241T, G242K, G242L, G242M, G242P, G242T, G242Y, T243I, T243M, T243R, T243V, V244A, V244E, V244G, V244L, V244R, T245E, T245G, T245L, T245M, T245N, T245Q, T245R, T245S, T246D, T246E, T246G, T246K, T246V, A247D, A247E, A247K, A247N, A247P, A247Q, A247R, A247S, A247V, A247W, S248A, S248E, S248F, S248H, S248I, S248L, S248Q, S248T, S248Y, K249A, K249D, K249G, K249H, K249I, K249L, K249M, K249N, K249P, K249Q, K249S, K249T, K249V, K249Y, S250H, S250M, S250W, I251F, I251L, I251V, I251W, I251Y, A252C, A252E, A252F, A252H, A252I, A252P, A252R, A252S, A252W, A252Y, G254D, G254F, G254I, G254L, G254M, G254Q, G254R, G254W, A255C, A255F, A255K, A255L, A255M, A255S, A255T, A255W, A255Y, S256A, S256C, S256F, S256G, S256K, S256L, S256M, S256N, S256Q, S256R, S256V, S256W, S256Y, A257D, A257G, A257I, A257N, A257T, A257V, D258A, D258L, D258M, D258W, V259E, V259L, V259S, V259T, T260A, T260D, T260G, T260I, T260K, T260V, S261A, S261D, S261H, S261R, S261W, S261Y, T262D, T262E, T262F, T262G, T262H, T262L, T262P, T262W, I263A, I263C, I263G, I263L, I263S, I263V, T264F, T264G, T264K, T264L, T264M, T264P, T264Q, T264R, T264S, T264Y, A265G, A265I, A265K, A265R, A265S, A265V, A266D, A266E, A266G, A266K, A266L, A266M, A266P, A266Q, A266S, S267A, S267D, S267K, S267M, S267N, S267P, S267Q, S267R, S267V, P268F, P268G, P268M, P268R, P268V, P268W, P268Y, K269G, K269R, K269V, K269Y, L270D, L270M, L270N, L270R, L270V, W271T, S272E, S272G, S272K, S272L, S272N, S272T, S272W, 1273K, I273L, I273P, I273R, I273S, I273W, K274D, K274P, K274Q, K274R, N275K, N275M, N275V, N275W, N277F, N277K, N277R, L278A, L278G, L278H, L278I, L278K, L278M, L278P, L278Q, L278R, L278S, L278V, Y279M, Y279T, Y279W, T280A, T280D, T280E, T280F, T280H, T280M, T280Q, V281A, V281I, V281L, V281Q, R282E, R282F, R282H, R282I, R282K, R282N, R282S, R282T, R282V, R282W, T283M, T283R, T283V, E284A, E284D, E284F, E284H, E284L, E284M, E284N, E284Q, E284R, E284Y, V285H, V285I, V285T, L286A, L286C, L286D, L286F, L286N, L286R, L286T, L286W, L286Y, N287I, N287L, G288F, G288L, G288S, V291C, V291D, V291F, V291G, V291H, V291L, V291P, V291S, V291T, V291Y, L292A, L292D, L292E, L292H, L292Q, L292S, L292V, D293C, D293E, D293F, D293I, D293S, D293V, D293W, T294C, T294G, T294M, T294Q, T294S, Y295F, Y295G, Y295I, Y295L, Y295R, Y295S, Y295W, D296F, D296H, D296K, D296L, D296N, D296R, D296V, T297G, T297I, T297Q, T297R, T297S, E298G, E2981, E298L, E298M, E298R, E298T, Y299F, Y299S, F301C, F301L, R302E, R302I, R302K, R302M, R302N, R302T, W303A, W303C, W303D, W303F, W303S, W303T, T304D, T304E, T304I, T304K, T304P, T304S, T304W, G305E, G305F, G305L, G305M, G305N, G305P, G305T, G305W, F306D, F306K, F306L, F306S, D307A, D307E, D307F, D307G, D307L, D307M, D307N, D307Q, D307S, D307V, D307W, A308C, A308E, A308G, A308I, A308M, A308Y, T309C, T309D, T309E, T309I, T309K, T309M, T309S, T309V, S310A, S310C, S310G, S310H, S310I, S310L, S310M, S310N, S310R, S310V, G311E, G311F, G311L, G311Q, G311R, G311V, F312H, F312L, F312R, F312V, L314A, L314C, L314T, L314V, L314Y, N315S, G316A, G316C, G316N, G316R, G316S, G316W, G316Y, E317D, E317H, E317I, E317K, E317V, E317W, E317Y, K318C, K318E, K318F, K318G, K318H, K318L, K318M, K318N, K318P, K318Q, K318V, K320C, K320E, K320F, K320I, K320L, K320R, K320T, K320V, K320W, L321C, L321G, L321H, L321M, L321V, L321Y, K322C, K322F, K322I, K322N, K322P, K322Q, K322R, K322S, V324E, V324G, V324T, S325D, S325G, S325T, M326E, M326G, M326S, M326T, M326V, M326Y, H328C, H328D, H328F, H328G, H3281, H328L, H328M, H328R, H328T, D329S, D329T, G331V, A335C, A335G, A335L, A337D, A337E, A337G, A337L, A337Q, N338P, N338R, N338V, R340A, R340C, R340L, A341M, A341S, I342A, I342G, I342K, I342P, E343A, E343H, E343N, E343R, E343S, E343T, E343Y, R344G, R344L, R344Y, Q345F, Q345G, Q345K, Q345N, Q345S, V346A, V346C, V346F, V346I, V346L, V346S, E347A, E347C, E347D, E347F, E347I, E347R, E347S, I348A, I348D, I348G, I348M, I348Q, I348R, L349A, L349E, L349Q, L349S, L349V, Q350G, Q350N, Q350W, K351N, K351R, K351V, K351W, M352G, M352L, M352T, M352W, G353K, V354E, V354G, V354S, V354W, N355H, N355M, N355R, N355W, I357T, T359E, T359L, T360V, N362G, N362S, N362T, P363A, P363D, P363I, P363L, P363M, P363S, P363V, A364C, A364E, A364F, A364G, A364I, A364M, A364P, A364V, A364W, A365C, A365E, A365I, A365P, A365V, A365W, K366A, K366D, K366E, K366I, K366L, K366M, K366P, K366S, K366V, A367C, A367I, A367N, A367Q, L368A, L368E, L368Q, L368S, L368V, L368Y, I369A, I369D, I369E, I369G, I369K, I369M, I369R, I369V, I369W, D370C, D370L, D370Q, D370R, D370S, D370T, V371D, V371F, V371G, V371I, V371L, V371Q, V371S, C372P, N373G, N373L, N373R, E374K, E374L, E374R, K375D, K375I, K375N, K375Q, K375S, G376A, G376S, V377A, V377M, V377T, L378I, L378P, L378W, L378Y, V379A, V379C, V379M, V379N, V380M, V380P, V380S, E381A, E381C, E381G, E381L, E381Q, E381T, V383A, V383K, V383L, M386G, M386N, M386Q, M386S, M386V, W387H, W387L, N388A, N388E, N388L, N388R, R389A, R389C, R389E, R389K, R389M, R389N, R389Q, R389S, R389T, R389V, S390C, S390D, S390G, S390H, S390N, S390P, S390Q, S390T, S390V, K391E, N392D, G393A, G393E, G393N, G393R, G393S, G393V, N394L, T395A, T395C, T395F, T395H, T395I, T395M, T395N, T395Q, T395S, T395W, E396K, E396L, E396M, E396V, E396W, Y398M, Y398N, G399S, K400A, K400C, K400D, K400E, K400M, K400N, K400P, K400Q, K400S, K400T, K400V, W401F, W401H, W401K, W401L, W401M, W401R, F402T, F402W, F402Y, G403A, G403D, G403H, G403K, G403P, G403Q, G403S, G403T, G403V, G403Y, Q404F, Q404H, Q404L, Q404M, Q404P, Q404R, Q404S, Q404V, A405C, A405E, A405H, A405K, A405P, A405R, A405V, I406C, I406D, I406N, A407C, A407G, A407M, A407Q, A407S, A407T, A407W, G408D, G408I, G408M, G408N, G408W, D409N, D409W, N410C, N410R, N410Y, A411E, A411N, A411R, A411S, A411V, V412M, V412S, L413D, L413E, L413F, L413I, L413P, L413T, G414A, G414C, G414M, G414N, G414R, G414T, G414W, G415A, G415Q, G415R, D416I, D416M, D416R, D416T, D416Y, K417C, K417F, K417G, K417R, K417T, D418L, D418P, D418R, D418Y, E419M, E419R, E419W, T420E, T420F, T420G, T420K, T420R, T420V, W421L, W421Q, W421S, A422P, A422T, A422V, K423D, K423L, K423M, K423R, F424C, F424L, F424N, F424T, D425E, L426C, L426M, L426Q, T427D, T427F, T427G, T427K, T427M, T427P, T427Q, T427R, T427S, T427W, S428F, S428K, S428W, T429D, T429P, I430C, I430D, I430E, I430L, I430M, I430Q, I430S, I430T, I430W, N431D, N431E, N431G, N431L, N431M, N431R, N431V, N431Y, R432A, R432E, R432F, R432G, R432N, R432Q, R432V, R432Y, D433C, D433G, D433H, D433I, D433P, D433Q, D433W, R434L, R434M, R434N, R434P, R434S, R434T, R434V, N435E, N435F, N435H, N435K, N435L, N435M, N435R, N435V, N435W, A436C, A436D, A436E, A436G, A436I, A436L, A436M, A436Q, A436S, P437A, P437D, P437K, P437L, P437Q, P437R, P437S, P437V, P437W, S438G, V439C, V439E, V439G, V439I, V439K, V439Q, V439T, V439Y, I440C, I440D, I440F, I440K, I440P, I440R, I440S, I440T, I440V, I440W, M441A, M441E, M441G, M441Q, M441R, M441T, M441V, W442E, W442G, W442M, W442P, W442Q, W442R, S443C, S443D, S443G, S443M, S443Q, S443Y, L444C, L444D, L444E, L444F, L444G, L444H, L444K, L444Q, L444V, L444W, G445A, G445C, G445V, N446D, N446T, M448A, M448C, M448D, M448E, M448I, M448L, M448P, M448Q, M448S, M448V, M448W, M449D, M449E, M449F, M449T, M449V, M449W, M449Y, E450F, E450S, E450T, E450V, E450W, G451C, G451F, G451L, G451P, G451V, G451W, I452G, I452K, I452L, I452M, I452Q, I452S, I452V, S453C, S453F, S453G, S453H, S453L, S453M, S453N, S453P, S453Q, S453R, S453V, G454L, G454W, S455A, S455E, S455K, S455M, S455P, S455R, S455V, S455W, V456A, V456D, V456E, V456F, V456K, V456L, V456W, S457E, S457H, S457K, S457L, S457M, S457P, S457Q, S457T, S457V, G458A, G458C, G458F, G458L, G458P, G458Q, G458S, G458V, G458W, F459A, F459C, F459E, F459G, F459N, F459R, F459S, F459T, F459W, P460C, P460M, P460Q, P460W, P460Y, A461D, A461G, A461M, A461N, A461Q, A461S, A461V, A461Y, T462C, T462E, T462F, T462L, T462M, T462S, S463G, S463K, S463Q, S463R, S463T, S463V, A464E, A464H, A464L, A464M, A464P, A464V, A464W, K465C, K465F, K465G, K465L, K465Q, K465R, K465V, K465W, K465Y, L466A, L466C, L466D, L466E, L466F, L466G, L466M, L466P, L466Q, L466S, L466V, L466Y, V467A, V467C, V467D, V467E, V467G, V467T, V467W, A468D, A468E, A468F, A468K, A468L, A468P, A468S, A468V, A468W, W469A, W469C, W469D, W469G, W469L, W469M, W469R, W469V, W469Y, T470E, T470L, T470M, T470Q, K471F, K471G, K471Q, K471W, K471Y, A472G, A472Y, A473E, A473M, A473P, D474A, D474C, D474E, D474K, D474M, D474R, D474W, S475E, S475F, S475Q, S475T, S475V, T476C, T476L, T476S, R477A, R477C, R477G, R477L, P478A, P478D, P478G, P478L, P478V, M479G, M479I, M479R, M479W, T480C, T480G, T480Q, K485E, K485R, K487A, K487C, K487F, K487G, K487N, K487S, K487W, A488C, A488G, A488H, A488L, A488N, A488S, A488V, N491A, N491E, N491W, E492A, E492W, S493E, S493G, S493H, S493L, S493M, S493Q, S493V, N494A, N494I, N494M, N494R, N494V, T495K, T495R, T495V, T495W, M496A, M496F, M496T, G497D, D498A, D498C, D498M, D498S, N499K, N499R, N499T, N499Y, L500A, L500E, L500N, L500V, T501C, T501G, T501M, A502L, A502Q, N503A, N503E, N503M, N503S, G504H, G504K, G504P, G505A, G505D, G505E, G505H, G505L, G505N, G505R, G505S, G505V, V506C, V506D, V506E, V506G, V506I, V506L, V506P, V506R, V506S, V506T, V506W, V507A, V507F, V507G, V507L, V507N, V507P, V507R, V507S, V507T, G508C, G508E, T509A, T509D, T509E, T509I, T509K, T509M, T509Q, T509S, T509V, T509Y, N510A, N510F, N510I, N510Q, Y511A, Y511K, S512C, S512E, S512F, S512G, S512I, S512M, S512Q, S512T, S512V, S512Y, D513C, D513G, D513K, D513L, D513M, D513P, D513Q, D513R, D513W, G514F, G514L, G514N, G514P, G514R, A515C, A515D, A515E, A515F, A515G, A515K, A515R, A515S, N516C, N516E, N516G, N516I, N516M, N516Q, N516S, N516T, N516V, Y517G, Y517I, Y517N, D518Q, D518Y, K519C, K519E, K519F, K519G, K519I, K519L, K519M, K519N, K519Q, K519S, K519T, I520A, I520C, I520G, I520H, I520M, I520N, I520Q, I520S, I520T, I520W, I520Y, R521A, R521C, R521K, R521N, R521Q, R521V, T522G, T522I, T522N, T523A, T523M, H524R, H524V, P525D, P525G, P525R, P525T, P525V, S526G, S526I, S526W, W527A, W527E, W527G, W527H, W527N, W527R, W527S, W527V, A528C, A528E, A528G, A528I, A528L, I529F, I529G, I529L, I529Y, Y530A, Y530M, G531E, G531S, G531T, T534A, T534I, T534Q, A535E, A535G, A535I, A535M, A537D, A537M, A537P, A537S, I538H, I538M, N539G, N539W, S540E, S540G, S540M, G542E, G542Q, G542S, G542T, I543V, I543W, N545G, N545I, N545Q, N545S, R546A, R546C, R546L, R546P, R546S, T547A, T547D, T547H, T547K, T547N, T547S, T548D, T548E, T548F, T548K, T548L, T548P, T548W, G549D, G549F, G549P, G549W, G550Q, G550R, G550S, A551D, A551I, A551Q, S553C, S553F, S553N, S553P, S553R, S553T, S553V, S554F, S554N, S554R, S554T, S554V, S554W, D555E, D555P, D555S, K556A, K556C, K556R, K556W, Q557F, Q557R, Q557S, L558E, L558H, L558I, L558P, T559A, T559G, T559I, T559Q, T559V, T559Y, S560P, S560V, Y561R, N563R, N563S, S564A, S564C, S564F, S564W, A565C, A565D, A565E, A565F, A565G, A565I, A565L, A565M, A565R, A565S, A565T, A565V, A565W, G567N, G567Q, G567V, A570G, A570K, A570L, A570M, A570S, A570T, A570W, V571W, A572S, A572W, S573G, S573K, S573Y, S574K, S574Q, S574V, S574W, A575D, A575M, A575V, W576A, W576F, W576V, W576Y, Y577G, Y577I, Y577L, Y577R, D578E, D578M, D578N, D578T, V579E, V579G, V579L, V579T, V580A, V580D, V580E, V580K, V580L, V580S, Q581F, Q581G, Q581P, Q581R, Q581S, Q581T, Q581Y, R582A, R582D, R582G, R582I, R582L, R582Y, D583V, D583W, F584E, F584I, F584W, V585I, V585M, V585Q, A586C, A586D, A586H, A586K, G587A, G587C, T588C, T588D, T588G, T588I, T588L, T588M, T588P, Y589A, Y589I, Y589Q, Y589V, Y589W, V590A, V590H, V590I, W591F, T592C, T592L, T592Q, T592S, G593C, G593I, F594C, F594I, F594L, F594M, D595E, D595Q, D595S, L597C, L597D, L597E, L597T, G598N, P600A, P600E, P600G, P600S, N604E, N604S, G605R, T606S, G607A, G607Q, G607S, S608E, S608I, S608M, S608N, S608Q, S608T, S608V, G609A, G609H, G609L, G609N, G609R, G609S, A610D, A610F, A610M, A610T, V611K, G612N, G612T, G612V, S613A, W614A, W614F, W614P, P615L, P615S, P615V, S616A, S616W, N619A, N619I, N619L, N619S, S620G, Y621W, I624A, I624V, V625A, V625E, V625F, V625M, V625Y, T627F, T627G, T627K, T627Q, A628C, A628D, A628N, G629T, F630A, F630C, F630D, F630G, F630S, F630Y, P631A, P631D, P631G, P631H, P631S, P631V, P631Y, K632C, K632D, K632G, K632T, D633G, T634A, T634E, T634F, T634S, T634V, Y635R, Y636K, F637C, F637G, F637I, F637S, F637T, F637V, Y638A, Y638W, Q639D, Q639N, Q639R, S640C, S640D, Q641T, W642I, N643R, D644C, D644G, D644Y, D645S, D645V, V646C, V646L, V646N, V646R, V646S, H647G, H647V, T648C, L649V, H650E, H650F, H650R, I651F, I651T, I651V, L652C, L652D, L652V, L652W, P653Q, A654D, A654K, A654M, A654R, W655F, W655R, N656A, N656K, N656V, E657K, E657R, E657V, V659D, V659N, A661E, A661G, A661H, A661K, A661L, A661M, A661Q, A661W, K662H, K662S, K662V, K662W, K662Y, N667L, N667R, P669A, P669E, P669F, P669L, P669R, P669T, P669W, V670C, V672L, Y673E, Y673G, Y673I, Y673R, Y673S, Y673W, T674C, T674D, T674G, T674H, T674M, T674Q, D675A, D675E, D675P, D675Q, D675S, D675V, D675Y, A676C, A676E, A676G, A676K, A676L, A676P, A676S, A676W, A677E, A677G, A677L, A677R, A677T, A677V, A677Y, K678A, K678C, K678T, K678V, V679G, V679Q, V679S, V679T, V679Y, K680A, K680E, K680G, K680H, K680I, K680L, K680N, K680Q, K680S, K680V, K680W, L681E, L681F, L681G, L681M, L681S, L681T, Y682D, Y682E, Y682I, Y682M, Y682S, Y682V, Y682W, F683H, F683L, F683M, F683Q, F683R, F683W, T684A, T684D, T684G, T684L, T684R, T684S, T684V, P685A, P685E, P685I, P685L, P685W, K686A, K686E, K686I, K686M, K686T, K686V, G687A, G687K, G687N, G687P, G687Q, G687R, S688C, S688D, S688E, S688G, S688K, S688L, S688P, S688T, T689A, T689D, T689E, T689G, T689L, T689P, T689S, T689W, T689Y, E690D, E690M, E690P, E690T, E690V, K691E, K691H, K691N, K691P, K691R, K691S, R692G, R692H, R692I, R692L, R692P, R692S, R692T, R692V, R692W, L693A, L693M, L693P, I694L, I694W, G695C, G695K, G695L, G695R, G695W, E696A, E696L, E696R, K697A, K697E, K697G, K697R, K697V, K697W, S698C, S698D, S698E, S698I, S698L, S698M, S698P, S698Q, S698R, S698T, T700A, T700C, T700D, T700E, T700G, T700K, T700Y, K701A, K701D, K701E, K701G, K701H, K701L, K701M, K701P, K701S, K701W, T703E, T703I, T703K, T703W, T704M, T704R, T704Y, A705C, A705D, A705E, A705K, A705N, A705P, A705R, A705V, A705W, A706C, A706G, A706T, A706W, A706Y, Y708C, Y708F, Y708G, Y708K, Y708L, Y708P, Y708T, T709M, Y710C, Y710D, Y710E, Y710G, Y710M, Y710N, Y710T, Y710V, Y710W, Q711A, Q711D, Q711G, Q711L, Q711M, Q711T, Q711Y, V712G, V712M, V712P, V712Q, V712R, Y713A, Y713E, Y713G, Y713L, Y713Q, E714D, E714H, E714I, E714K, E714M, E714N, E714R, E714S, E714V, E714Y, G715K, A716C, A716G, A716L, A716M, A716P, A716R, A716V, D717C, D717G, D717L, D717S, K718A, K718L, K718Q, K718T, K718W, K718Y, D719E, D719L, D719P, D719S, D719T, D719V, S720A, S720M, S720R, S720Y, T721D, T721H, T721K, T721L, T721N, T721P, T721Q, T721S, T721V, T721W, T721Y, A722Q, A722T, H723G, H723P, K724A, K724G, K724R, N725A, N725D, N725I, M726A, M726D, M726E, M726K, M726Q, M726V, M726W, Y727L, Y727T, L728C, L728K, L728Q, L728S, L728T, L728W, T729A, T729E, T729M, W730A, N731A, N731E, N731Q, N731S, N731Y, V732G, V732P, V732R, V732W, P733A, P733R, P733W, W734D, W734G, W734R, W734V, A735Q, A735R, A735S, A735T, A735W, E736S, G737F, G737K, G737N, G737Y, T738G, T738L, T738S, I739E, I739K, I739M, I739W, I739Y, S740D, S740E, S740F, S740H, S740L, A741I, A741P, A741S, A741V, E742D, E742M, E742Q, E742V, A743C, A743E, A743H, A743I, A743L, Y744A, Y744E, Y744I, Y744K, Y744L, Y744R, D745C, D745F, D745N, D745R, E746A, E746C, E746K, E746T, N747E, N747F, N747G, N747P, N747R, N748S, R749H, R749M, R749S, R749T, L750G, L750M, L750P, L750Q, L750S, I751C, I751H, I751Q, I751S, P752A, P752C, P752H, P752L, P752S, P752V, P752Y, E753Q, G754H, G754I, G754P, S755C, S755I, S755T, T756E, T756N, T756P, T756Q, T756S, T756W, E757A, G758A, G758V, N759D, N759R, N759S, N759V, A760G, A760N, A760P, A760Q, S761A, S761K, S761Q, V762D, V762G, V762K, V762W, T765A, T765P, T765R, T765W, G766M, G766S, A768H, A769F, A769G, A769I, A769N, A769V, A769Y, K770H, L771A, L771I, K772A, K772C, K772P, K772V, K772W, A773C, A773G, A773H, A773M, A773R, A773S, A773V, D774A, D774R, A775L, A775V, A775W, D776I, D776L, D776R, D776S, D776V, R777D, R777E, R777G, R777H, R777P, R777S, R777T, K778A, K778C, K778F, K778G, K778L, K778N, K778R, T779C, T779I, I780V, T781A, T781C, T781E, T781F, T781G, T781M, T781P, T781R, T781Y, A782C, A782E, A782K, A782N, A782P, A782Q, A782Y, D783A, D783C, D783E, D783R, G784A, G784F, G784L, G784S, G784T, K785C, K785I, K785S, K785V, K785W, K785Y, D786S, D786V, L787D, L787K, L787P, L787T, L787Y, S788A, S788G, S788I, Y789C, Y789D, Y789I, Y789V, I790A, I790C, I790F, I790R, I790V, E791A, E791D, E791F, E791M, E791S, E791T, E791V, E791W, V792C, V792G, V792L, V792S, V792Y, D793C, D793F, D793H, D793K, D793N, D793Y, V794C, V794D, V794L, V794Q, V794T, V794W, T795P, T795Y, D796M, D796Q, D796S, D796T, A797H, A797K, N798G, N798I, N798P, N798Q, G799D, G799K, G799L, G799M, G799Q, G799Y, H800A, H800F, H800G, H800L, H800S, H800V, I801C, I801E, I801W, V802E, V802I, V802P, V802S, V802Y, P803A, P803F, P803G, P803K, P803S, P803Y, D804E, D804G, D804K, D804N, D804S, A805C, A805F, A805G, A805I, A805N, A805P, A806F, A806I, A806Q, N807F, N807Q, N807V, N807W, R808C, R808F, R808G, R808I, R808N, R808P, R808Q, V809A, V809C, V809L, V809M, V809P, T810L, T810P, T810Q, T810R, T810Y, F811L, F811Y, D812E, D812F, D812I, D812Q, V813F, V813H, V813T, V813W, V813Y, K814G, K814H, K814I, K814L, K814P, G815A, G815F, G815M, G815P, G815V, A816C, A816D, A816F, A816I, A816N, A816V, A816W, G817C, G817H, G817I, G817N, G817S, K818D, K818F, K818L, K818Q, K818R, K818S, K818V, K818W, K818Y, L819F, L819W, V820C, V820F, V820I, V820K, V820R, V820W, G821A, G821C, G821E, G821F, G821I, G821K, G821M, G821N, G821V, G821Y, V822A, V822D, V822E, V822T, D823E, N824A, N824C, N824G, N824Q, G825A, S826A, S826F, S826G, S826I, S826L, S826R, S826W, S827C, S827Q, P828C, P828G, P828I, P828L, P828Y, D829C, D829I, D829S, D829T, D829V, H830E, H830G, H830M, H830P, H830Q, H830R, H830V, D831A, D831F, D831G, D831I, D831M, D831P, D831R, D831V, S832E, S832F, S832G, S832L, S832M, S832P, S832R, S832V, S832W, Y833C, Y833D, Y833E, Y833I, Y833K, Y833N, Y833P, Y833V, Q834F, Q834G, Q834M, A835D, A835E, A835F, A835H, A835K, A835W, D836C, D836E, D836H, D836Q, D836R, D836S, D836T, D836V, D836W, D836Y, N837D, N837F, N837G, N837H, N837L, N837P, N837T, R838D, R838F, R838G, R838K, R838M, R838N, R838S, R838W, K839A, K839C, K839D, K839E, K839G, K839L, K839N, K839P, A840G, A840I, A840P, A840V, F841C, F841D, F841I, F841K, F841W, S842A, S842L, S842M, G843A, G843C, K844A, K844G, K844L, K844W, K844Y, V845N, V845W, L846G, L846I, L846M, L846S, A847L, A847T, I848M, V849A, V849L, V849S, V849T, Q850C, Q850G, Q850I, Q850L, Q850T, Q850V, Q850Y, S851C, S851D, S851E, S851L, S851T, T852D, T852G, T852L, K853N, K853P, K853Q, K853V, E854C, E854I, E854M, E854R, A855K, A855V, A855Y, E857P, E857V, I858D, I858E, I858F, I858G, I858K, I858M, I858P, I858Q, I858Y, T859V, V860T, V860Y, T861F, T861I, T861Q, T861V, T861W, A862C, A862P, A862V, K863F, K863I, K863L, K863N, K863W, A864E, A864H, A864K, A864L, D865A, D865G, G866H, G866R, L867W, S870E, S870M, S870R, S870T, T871P, V872C, V872G, K873G, K873Y, I874G, A875R, T877A, V879P, V879S, P880S, G881W, T882A, T882M, T882R, S883L, T884A, E885V, K886E, K886L, K886V, K886W, T887A, T887D, T887F, T887G, T887N, T887R, T887V, V888A, V888D, V888G, R889G, Y892D, Y892P, Y892R, Y893E, Y893G, S894D, S894G, R895M, N896M, Y897V, Y898T, V899G, K900E, K900G, T901G, T901Q, T901R, T901V, T901Y, G902A, G902D, G902F, G902L, G902P, G902Q, G902R, G902S, G902W, N903D, N903I, K904D, K904E, K904M, K904N, K904R, K904S, K904V, K904W, P905A, P905C, P905R, P905V, P905W, P905Y, I906A, I906D, I906S, I906T, I906W, I906Y, L907F, L907S, L907Y, P908C, P908D, P908G, P908I, P908L, P908M, P908T, S909E, S909F, S909G, S909W, S909Y, D910C, D910I, D910S, D910W, V911A, V911S, E912A, E912K, E912L, E912T, E912V, V913G, V913Q, V913R, V913W, R914A, R914E, R914F, R914I, R914K, R914V, R914W, R914Y, Y915A, Y915C, Y915G, Y915I, Y915M, Y915Q, Y915V, S916G, D917C, D917E, D917F, D917L, D917M, D917Q, D917S, G918E, G918H, G918T, G918V, G918W, T919D, T919K, T919Q, T919W, T919Y, S920C, S920E, S920M, S920P, S920R, S920V, S920W, D921C, D921P, D921Q, D921V, R922A, R922G, R922M, R922V, R922W, Q923A, Q923C, Q923E, Q923L, Q923M, Q923V, Q923W, N924A, N924L, N924P, N924Q, N924S, N924W, V925A, V925C, V925E, V925G, V925K, V925N, V925S, V925W, T926G, T926P, T926R, T926S, T926V, T926W, W927C, W927G, W927P, D928A, D928E, D928H, D928L, D928Q, A929C, A929P, A929V, V930A, V930E, V930I, V930K, V930M, V930T, S931G, S931P, S931R, D932F, D932R, D932S, D932T, D932V, D933I, D933R, D933S, Q934S, Q934V, I935A, I935C, I935D, I935E, I935L, I935P, I935V, I935W, A936I, A936L, A936Q, A936R, A936Y, K937G, K937I, K937M, K937P, K937Q, K937R, K937V, A938C, A938H, A938N, A938T, A938V, A938W, G939D, G939K, S940C, S940E, S940M, S940R, S940T, S940V, S940W, F941C, F941M, F941W, S942A, S942E, S942K, S942L, S942P, S942T, S942V, V943A, V943G, V943H, V943Q, V943R, A944D, A944G, A944H, A944P, A944R, A944V, G945E, G945P, G945T, T946A, T946E, T946G, T946L, T946P, T946V, T946W, V947G, V947H, V947L, V947M, V947P, V947R, V947T, A948C, A948I, A948R, A948W, G949A, G949F, G949V, Q950D, Q950G, Q950K, Q950M, Q950W, K951D, K951G, K951P, K951Q, K951S, K951W, K951Y, I952H, I952Q, S953F, S953M, S953N, S953R, S953W, V954D, V954L, V954Q, V954S, V954T, R955A, R955C, R955E, R955K, R955Q, R955W, V956A, V956D, V956G, V956H, V956I, V956M, V956Q, V956W, T957D, T957S, T957W, M958D, M958I, M958K, I959A, I959L, I959S, I959V, I959Y, D960G, D960H, D960L, D960P, D960S, D960W, E961D, E961F, E961K, E961P, E961S, E961T, I962A, I962C, I962D, I962G, I962K, I962N, I962Q, I962T, G963C, G963E, G963L, G963P, A964C, A964E, A964H, L965C, L965E, L965G, L965K, L965M, L965P, L965Q, L965S, L965V, L965Y, L966A, L966G, L966H, L966K, L966N, L966P, L966Q, L966S, L966T, L966V, N967C, N967D, N967I, N967L, N967M, N967P, N967S, N967T, N967V, Y968G, Y968L, Y968Q, Y968V, S969C, S969D, S969G, S969H, S969I, S969L, S969M, S969P, S969Q, S969Y, A970I, A970L, S971E, S971F, S971G, S971H, S971V, S971W, P973C, P973D, P973K, P973N, P973Q, P973R, P973V, P973W, P973Y, V974C, V974E, V974G, V974N, V974T, V974Y, G975D, G975F, G975K, G975L, G975Q, G975V, T976D, T976F, T976G, T976K, T976L, T976P, T976S, T976Y, P977A, P977C, P977K, P977R, P977T, P977Y, A978F, A978G, A978M, A978N, A978P, A978R, A978S, A978Y, V979G, V979N, V979R, V979Y, L980A, L980F, L980H, L980I, L980K, L980N, L980Q, L980T, L980Y, P981L, P981M, G982A, G982H, G982M, G982P, G982Q, G982W, R984P, R984S, P985E, P985F, P985H, P985K, P985L, P985W, A986C, A986E, A986F, A986I, A986K, A986L, A986M, A986N, A986S, A986W, V987A, V987C, V987F, V987I, V987K, V987L, V987Q, V987T, L988A, L988C, L988E, L988G, L988H, L988M, L988Q, L988R, L988S, L988V, L988Y, P989A, P989C, P989D, P989G, P989H, P989I, P989M, P989N, P989Q, P989W, D990F, D990P, D990S, D990W, G991C, G991F, G991H, G991K, G991P, G991Y, T992E, T992H, T992M, T992N, T992Y, V993D, V993G, V993N, V993S, T994I, T994S, T994V, S995E, S995L, S995R, S995V, A996Q, A996R, A996V, N997A, N997C, N997E, N997K, N997L, N997S, N997V, N997W, N997Y, F998M, F998W, A999F, A999G, A999L, A999M, A999R, A999S, V1000C, V1000L, V1000M, V1000N, V1000P, V1000W, D1001G, D1001K, D1001L, D1001M, D1001Q, D1001S, D1001T, D1001V, D1001Y, W1002A, W1002D, W1002E, W1002H, W1002N, W1002P, W1002Q, W1002S, T1003F, T1003G, T1003L, T1003N, T1003P, T1003R, T1003S, T1003W, T1003Y, K1004D, K1004E, K1004F, K1004G, K1004H, K1004M, K1004P, K1004R, K1004S, K1004V, P1005I, P1005N, P1005Q, P1005V, P1005Y, A1006C, A1006I, A1006N, A1006P, A1006S, A1006V, A1006W, A1006Y, D1007C, D1007L, D1007P, D1007V, T1008G, V1009G, V1009S, Y1010A, Y1010P, Y1010R, Y1010T, N1011A, N1011S, N1011T, N1011W, T1012E, T1012H, T1012I, T1012Q, T1012Y, A1013D, A1013K, A1013Q, A1013T, A1013V, G1014E, G1014I, G1014L, G1014M, G1014V, G1014W, G1014Y, T1015A, T1015F, T1015G, T1015V, V1016C, V1016D, V1016P, K1017E, K1017G, V1018I, V1018K, V1018L, V1018M, V1018R, V1018S, V1018W, T1021C, T1021E, T1021F, T1021G, T1021K, T1021L, T1021S, T1021V, A1022H, A1022L, A1022S, A1022Y, T1023D, T1023M, T1023Q, T1023R, V1024E, V1024G, V1024H, V1024K, V1024N, V1024R, V1024S, V1024W, G1026E, G1026H, G1026L, G1026R, G1026S, G1026V, G1026Y, K1027C, K1027N, K1027Q, K1027R, K1027V, E1028G, E1028S, E1028T, F1029I, F1029K, F1029L, F1029P, F1029V, F1029W, F1029Y, K1030D, K1030F, K1030H, K1030L, K1030M, K1030W, V1031H, V1031K, V1031Y, A1033G, A1033S, A1033V, T1034G, T1034H, T1034N, T1034W, I1035D, I1035G, I1035Q, R1036G, R1036L, R1036T, R1036Y, V1037C, V1037F, V1037P, V1037Q, Q1038A, Q1038D, Q1038K, R1039S, R1039V, S1040A, S1040M, S1040N, S1040R, S1040W, Q1041P, V1042N, T1043F, T1043G, T1043N, T1043R, I1044A, I1044L, G1045S, S1046I, S1046M, S1047D, V1048C, V1048F, V1048G, V1048I, V1048M, V1048Q, G1050L, G1050S, G1050V, N1051A, N1051E, N1051K, A1052C, A1052K, A1052M, A1052P, A1052R, L1053A, L1053W, R1054C, R1054L, R1054N, L1055R, L1055T, Q1057A, Q1057E, Q1057P, Q1057R, N1058R, N1058S, N1058V, N1058W, I1059W, P1060G, P1060N, P1060Q, P1060S, P1060T, A1061E, A1061G, A1061K, A1061W, D1062A, D1062F, D1062G, D1062I, D1062L, D1062M, D1062P, D1062S, K1063D, K1063M, Q1064C, Q1064M, Q1064R, Q1064T, Q1064V, S1065A, S1065C, S1065E, S1065G, S1065T, S1065W, D1066A, D1066G, D1066M, D1066V, D1066W, T1067G, T1067M, L1068C, L1068E, L1068P, L1068Q, L1068Y, D1069G, D1069K, D1069R, D1069W, A1070P, A1070T, I1071M, I1071R, I1071W, K1072E, K1072G, K1072P, K1072Q, K1072S, D1073F, D1073L, D1073M, D1073P, D1073W, G1074I, G1074L, G1074R, S1075C, S1075G, S1075I, S1075L, S1075V, T1076C, T1076E, T1076H, T1076Q, T1076S, T1077K, T1077L, T1077R, V1078D, V1078E, V1078L, V1078W, D1079G, D1079L, N1081D, N1081E, N1081G, T1082A, T1082C, T1082E, T1082F, T1082G, T1082K, T1082N, T1082S, G1083E, G1083F, G1083L, G1083P, G1083S, G1084C, G1084M, G1084V, G1084W, G1084Y, G1085A, G1085P, G1085R, G1085S, A1086H, A1086K, A1086Q, A1086R, A1086T, N1087A, N1087E, N1087I, N1087R, N1087V, N1087W, P1088D, P1088E, P1088G, P1088R, P1088W, S1089C, S1089E, S1089G, S1089K, S1089Q, S1089R, S1089V, A1090F, A1090G, A1090I, A1090K, W1091A, W1091E, W1091G, W1091H, W1091T, W1091V, W1091Y, T1092A, T1092E, T1092G, T1092K, T1092Q, T1092S, T1092V, N1093A, N1093G, N1093L, N1093P, N1093Q, N1093T, N1093V, W1094D, W1094E, W1094P, W1094R, W1094T, A1095P, A1095R, A1095T, A1095W, Y1096A, Y1096D, Y1096H, Y1096L, Y1096R, S1097D, S1097E, S1097K, S1097L, S1097T, S1097W, K1098D, K1098F, K1098G, K1098Q, K1098S, A1099C, A1099D, A1099F, A1099S, A1099V, A1099W, G1100D, G1100E, G1100H, G1100M, G1100N, G1100T, H1101K, H1101L, H1101Q, H1101R, H1101V, N1102E, N1102F, N1102H, N1102K, N1102L, N1102Q, N1102R, N1102T, T1103A, T1103E, T1103H, T1103S, T1103W, A1104I, A1104K, A1104R, E1105L, E1105S, I1106T, I1106V, T1107C, T1107M, T1107R, T1107S, F1108D, F1108K, F1108L, F1108T, F1108W, E1109A, E1109D, E1109L, E1109W, A1111G, A1111S, E1113D, E1113G, E1113P, E1113V, Q1114E, Q1114I, Q1114L, Q1114R, Q1114S, Q1114T, Q1114V, Q1115A, Q1115K, Q1115L, Q1115P, Q1115T, Q1115W, L1116D, L1116G, L1116H, L1116K, L1116V, L1116W, G1117E, G1117I, G1117M, G1117R, G1117S, G1117T, G1117V, G1117W, Q1118A, Q1118M, Q1118S, Q1118T, Q1118W, I1119D, I1119E, I1119G, I1119N, I1119S, V1120N, V1120S, V1120T, M1121G, M1121K, M1121N, M1121P, M1121S, M1121V, M1121Y, Y1122A, Y1122C, Y1122I, Y1122K, Y1122R, Y1122V, Y1122W, F1123E, F1123H, F1123R, F1123T, F1124E, F1124R, F1124V, F1124W, R1125D, R1125E, R1125F, R1125K, R1125T, R1125V, R1125W, D1126H, D1126K, D1126L, D1126R, S1127F, S1127I, S1127K, S1127M, S1127Q, S1127T, S1127W, N1128A, N1128C, N1128R, N1128S, N1128T, N1128W, A1129E, A1129L, A1129N, A1129Q, A1129R, A1129V, V1130A, V1130G, V1130P, V1130R, V1130S, R1131A, R1131N, R1131Q, R1131S, R1131W, F1132E, F1132K, F1132M, F1132P, F1132Q, F1132T, P1133D, P1133G, P1133L, P1133Q, P1133R, P1133V, D1134E, D1134G, D1134L, A1135E, A1135K, A1135L, A1135M, A1135S, A1135W, A1135Y, G1136A, G1136E, G1136P, G1136Q, G1136T, K1137A, K1137C, K1137G, K1137L, K1137P, K1137Q, K1137R, K1137S, K1137T, K1137V, T1138R, T1138Y, K1139A, K1139L, K1139R, K1139T, I1140A, I1140C, I1140G, I1140L, I1140M, I1140P, I1140R, I1140T, Q1141A, Q1141C, Q1141G, Q1141K, Q1141N, Q1141P, Q1141T, Q1141W, I1142E, I1142R, I1142S, I1142W, I1142Y, S1143G, A1144C, A1144D, A1144E, A1144N, A1144P, A1144S, A1144T, A1144V, A1144W, D1145E, D1145R, D1145T, G1146A, G1146C, G1146D, G1146K, G1146L, G1146R, G1146V, K1147A, K1147G, K1147T, K1147V, N1148H, N1148I, N1148K, N1148P, N1148Q, N1148R, N1148S, N1148T, N1148W, W1149C, W1149G, W1149I, W1149K, W1149N, W1149Q, W1149S, W1149T, W1149V, W1149Y, T1150G, T1150K, T1150P, D1151C, D1151G, D1151R, D1151T, D1151W, L1152A, L1152C, L1152E, L1152Q, L1152W, A1153E, A1153G, A1153K, A1153L, A1154C, A1154D, A1154E, A1154G, A1154R, A1154S, T1155E, T1155L, T1155Q, T1155R, T1157V, T1157W, I1158R, I1158S, I1158W, A1159C, A1159E, A1159I, A1159P, A1159R, A1159V, A1160K, A1160L, A1160Q, A1160S, Q1161A, Q1161P, Q1161S, E1162A, E1162C, E1162D, E1162F, E1162I, E1162N, E1162Q, E1162T, E1162W, E1162Y, E1165H, E1165L, E1165M, E1165R, E1165S, E1165W, R1166D, R1166K, R1166Q, V1167A, V1167C, V1167L, V1167P, V1167R, K1168L, K1168Q, K1168R, K1168W, P1169M, P1169R, P1169S, Y1170E, Y1170K, Y1170M, Y1170Q, Y1170R, Y1170V, T1171A, T1171G, T1171M, T1171Q, T1171R, T1171S, Y1172D, Y1172E, Y1172H, Y1172I, Y1172K, Y1172L, Y1172S, Y1172V, D1173A, D1173E, D1173F, D1173G, D1173K, D1173L, D1173P, D1173R, D1173T, D1173W, F1174D, F1174P, F1174Q, F1174R, F1174S, F1174T, F1174V, F1174W, A1175G, A1175I, A1175N, A1175Q, A1175S, A1175V, A1175Y, V1177N, V1177P, V1177S, V1177T, G1178M, G1178Q, G1178S, G1178T, A1179L, A1179P, A1179Q, A1179W, T1180A, T1180G, T1180I, T1180L, T1180M, T1180Q, T1180S, T1180Y, F1181E, F1181L, F1181V, V1182M, K1183A, K1183E, K1183T, K1183V, V1184C, V1184E, V1184K, V1184L, V1184P, V1184Q, V1184Y, T1185Q, T1185V, V1186S, N1188V, N1188W, A1189C, A1189K, A1189T, A1189V, D1190R, D1190S, D1190T, D1190Y, T1191E, T1191L, T1192H, T1192P, T1193G, P1194A, P1194E, P1194G, P1194W, S1195G, V1197A, V1198E, C1199D, C1199T, A1200G, A1200W, L1202A, L1202C, T1203E, T1203K, E1204G, E1204S, I1205V, K1208R, K1208V, K1208W, T1209A, T1209C, T1209E, T1209K, T1209N, T1209Q, T1209R, T1209W, A1210D, A1210E, A1210G, A1210K, A1210L, A1210Q, A1210R, A1210T, A1210W, T1211C, T1211D, T1211E, T1211G, T1211H, T1211K, T1211P, T1211Q, T1211R, T1211S, T1211V, K1213A, K1213D, K1213S, K1213T, K1213W, F1214A, F1214E, F1214K, F1214L, F1214P, F1214R, F1214S, F1214V, V1215D, V1215E, V1215K, V1215L, V1215Q, V1215S, V1215W, T1216L, T1216P, T1216Q, T1216R, N1217A, N1217D, N1217E, N1217F, N1217P, N1217R, N1217S, N1217T, T1218A, T1218C, T1218E, T1218G, T1218Q, T1218S, T1218V, T1218W, S1219A, S1219E, S1219F, S1219I, S1219K, S1219R, S1219V, A1220C, A1220G, A1220L, A1220P, A1220R, A1220V, A1221D, A1221G, A1221K, A1221L, A1221R, A1221V, A1221W, L1222A, L1222C, L1222E, L1222F, L1222Q, L1222R, L1222V, L1222W, S1223C, S1223F, S1223G, S1223K, S1223L, S1223V, S1224A, S1224D, S1224G, S1224L, S1224M, S1224P, S1224R, S1224W, L1225C, L1225D, L1225E, L1225F, L1225G, L1225K, L1225P, L1225T, L1225V, L1225W, T1226A, T1226G, T1226M, T1226P, T1226R, T1226S, T1226V, T1226Y, V1227A, V1227C, V1227D, V1227E, V1227G, V1227L, V1227P, V1227Q, V1227S, N1228A, N1228D, N1228F, N1228K, N1228L, N1228T, G1229A, G1229C, G1229E, G1229Q, G1229S, G1229V, T1230F, T1230H, T1230I, T1230K, T1230L, T1230P, T1230R, T1230S, T1230W, K1231F, K1231G, K1231L, K1231M, K1231P, K1231S, K1231W, V1232E, V1232K, V1232Q, V1232R, V1232S, V1232T, V1232W, S1233P, S1233W, D1234G, D1234K, D1234R, D1234V, S1235D, S1235E, S1235G, S1235L, S1235P, S1235R, S1235W, S1235Y, V1236A, V1236C, V1236G, V1236I, V1236P, V1236Q, V1236R, L1237D, L1237E, L1237R, L1237V, L1237W, A1238D, A1238E, A1238K, A1238L, A1238N, A1238P, A1238R, A1238S, A1238T, A1239D, A1239P, A1239R, G1240D, G1240L, G1240N, G1240Q, G1240S, G1240T, G1240W, S1241D, S1241G, S1241I, S1241L, S1241M, S1241P, Y1242C, Y1242E, Y1242K, Y1242R, Y1242S, Y1242W, N1243C, N1243L, N1243M, N1243P, N1243Q, N1243S, N1243T, N1243V, N1243W, T1244A, T1244D, T1244E, T1244G, T1244L, T1244Q, T1244S, T1244V, T1244W, A1246F, A1246M, A1246N, A1246P, A1246Q, A1246R, A1246S, A1246T, I1247A, I1247G, I1247M, I1247Q, I1247R, I1247S, I1247T, I1247V, I1247W, I1248A, I1248G, I1248K, I1248L, I1248R, I1248S, I1248Y, A1249E, A1249G, A1249H, A1249I, A1249R, A1249T, A1249V, D1250I, D1250K, D1250S, D1250T, D1250W, D1250Y, V1251I, V1251T, V1251W, K1252D, K1252G, K1252V, K1252W, A1253P, A1253V, E1254F, E1254G, E1254H, E1254L, E1254R, E1254V, G1255H, G1255M, G1255S, G1255V, E1256G, E1256M, E1256N, E1256R, E1256V, E1256W, G1257F, G1257K, G1257L, G1257Q, G1257R, G1257W, N1258C, N1258G, N1258H, N1258K, N1258S, A1259K, A1259L, A1259W, V1261I, V1261L, V1261P, V1261Q, V1261R, V1261T, T1262A, T1262F, T1262M, T1262Q, T1262R, V1263E, V1263G, V1263Q, V1263R, V1263T, V1263W, L1264A, L1264E, L1264H, L1264R, L1264S, L1264Y, P1265C, P1265K, P1265L, P1265R, P1265S, P1265V, P1265W, A1266F, A1266L, A1266P, A1266S, A1266V, H1267A, H1267E, H1267F, N1269A, N1269E, N1269K, N1269R, N1269S, N1269T, N1269W, V1270D, V1270E, V1270G, V1270I, V1270L, V1270T, V1270W, I1271A, I1271H, I1271Q, R1272E, R1272F, R1272M, R1272P, R1272V, V1273R, I1274F, I1274M, I1274R, T1275A, T1275L, T1275W, E1276R, E1276W, S1277L, S1277T, S1277W, E1278Q, E1278R, D1279G, D1279I, D1279R, D1279T, D1279V, D1279W, H1280C, H1280E, H1280G, H1280V, H1280W, V1281F, V1281I, V1281S, V1281W, T1282D, T1282L, T1282V, R1283A, R1283D, R1283E, R1283P, R1283W, K1284G, T1285A, T1285E, T1285F, T1285G, T1285M, T1285R, T1285Y, F1286A, F1286E, F1286P, F1286R, F1286S, F1286T, T1287C, T1287K, T1287L, T1287M, T1287Q, T1287R, T1287S, T1287W, I1288A, I1288D, I1288F, I1288G, I1288K, N1289A, N1289Q, N1289T, L1290A, L1290G, L1290R, L1290V, L1290W, G1291K, G1291P, G1291V, G1291W, G1291Y, T1292G, T1292L, T1292Y, E1293G, E1293K, E1293L, E1293S, E1293V, Q1294E, Q1294L, Q1294P, Q1294W, E1295K, F1296A, F1296G, F1296I, P1297F, A1298Y, D1301I, E1302G, E1302R, E1302S, R1303S, D1304A or D1304V.

4. The lactase variant of claim 1, wherein the variant has a decreased or an increased galactose inhibition, preferably measured as a galactose inhibition improvement factor (GI-IF) of at least 1.1 or at most 0.9, compared to the lactase of SEQ ID NO: 1.

5. The lactase variant of claim 1, which has an amino acid sequence which is at least 85%, but less than 100%, identical to SEQ ID NO 1.

6. The lactase variant of claim 1, which is a variant of a lactase obtained from Bifidobacterium.

7. An isolated polynucleotide encoding the variant of claim 1.

8. A nucleic acid construct or expression vector comprising the polynucleotide of claim 7.

9. A recombinant host cell transformed with the polynucleotide of claim 7.

10. A method of producing a lactase variant, comprising:
a) cultivating the host cell of claim 9 under conditions suitable for expression of the variant; and
b) recovering the variant.

11. A method for producing a dairy product comprising:
a) providing a milk-based substrate comprising lactose; and
b) treating said substrate with the variant of claim 1.

12. (canceled)

13. A liquid composition comprising the variant of claim 1 and 20-70% glycerol.

14-15. (canceled)
